(19) United States
(12) Patent Application Publication
(10) Pub. No.: US 2024/0269263 A1
(43) Pub. Date: Aug. 15, 2024

(54) IMMUNOMODULATORY COMPOSITIONS AND RELATED METHODS

(71) Applicant: Flagship Pioneering Innovations VII, LLC, Cambridge, MA (US)

(72) Inventors: Ellen Lovisa Larsdotter AFZELIUS, Belmont, MA (US); Hozefa Saifuddin BANDUKWALA, Southborough, MA (US); Ryan Edward KOHN, Cambridge, MA (US); Sofia Marques Tuna Ribeiro BRITES BOSS, Boston, MA (US)

(21) Appl. No.: 18/433,923

(22) Filed: Feb. 6, 2024

Related U.S. Application Data
(60) Provisional application No. 63/483,440, filed on Feb. 6, 2023.
(60) Provisional application No. 63/492,605, filed on Mar. 28, 2023.
(60) Provisional application No. 63/502,870, filed on May 17, 2023.
(60) Provisional application No. 63/613,940, filed on Dec. 22, 2023.
(60) Provisional application No. 63/618,778, filed on Jan. 8, 2024.

(51) Int. Cl. A61K 39/215; A61K 9/51; A61K 39/00; A61K 39/39; A61P 37/04

(52) U.S. Cl. CPC A61K 39/215; A61K 9/5123; A61K 39/39; A61P 37/04; A61K 2039/53; A61K 2039/55527

(57) ABSTRACT

Provided herein are, inter alia, compositions (e.g., vaccine compositions (e.g., vaccine booster compositions)) comprising a hIL-10R binding agent (e.g., a hIL-10R binding protein (or a nucleic acid molecule comprising the same)) and optionally an immunogen (e.g., an immunogenic protein (or a nucleic acid molecule encoding the same)). Further provided herein are methods of utilizing hIL-10R binding agents (e.g., hIL-10R binding proteins (or nucleic acid molecules comprising the same)), including, e.g., in methods of vaccination, e.g., as vaccine boosters.

### RELATED APPLICATIONS

This application claims priority to U.S. Ser. No. 63/483,440, filed Feb. 6, 2023, U.S. Ser. No. 63/492,605, filed Mar. 28, 2023, U.S. Ser. No. 63/502,870, filed May 17, 2023, U.S. Ser. No. 63/613,940, filed Dec. 22, 2023, and U.S. Ser. No. 63/618,778, filed Jan. 8, 2024, the entire contents of each of which is incorporated herein by reference.

### 1. FIELD

This disclosure relates to compositions (e.g., vaccine booster compositions) comprising a hIL-10R binding agent (e.g., a hIL-10R binding protein (or a functional fragment or variant thereof) or a nucleic acid molecule encoding the same) and optionally an immunogen (e.g., an immunogenic protein or a nucleic acid molecule encoding the same). The disclosure further relates to methods of utilizing hIL-10R binding agents (e.g., hIL-10R binding proteins (or functional fragments or variants thereof) or a nucleic acid molecule encoding the same), including, e.g., in methods of vaccination, e.g., as vaccine boosters, methods of treating or preventing infection, and methods of inducing an immune response.

### 2. BACKGROUND

Vaccines are a critical class of therapeutics that are used to stimulate an immune response in a subject directed against a particular infectious agent (e.g., virus, bacterium) or an aberrant tissue (e.g., a tumor). As such, vaccines contain at least one immunogen that serves to activate the desired immune response. The form of the immunogen varies depending on the type of vaccine. For example, some vaccines utilize inactivated or live attenuated infectious agents (e.g., viruses), while others utilize a vector (e.g., plasmid, viral). Protein-based vaccines utilize a protein form of the immunogen, while nucleic acid-based vaccines (e.g., RNA (e.g., mRNA) or DNA-based vaccines) utilize the genetic material encoding the immunogen such that the cells within the body of a vaccinated subject can make the immunogen in vivo. In some vaccine regimens, a single immunization is sufficient to induce a protective immune response, while others require multiple immunizations for an optimized immune response. In addition to the immunogen, some vaccine regimens utilize one or more adjuvants to induce a stronger immune response (e.g., a longer duration, greater magnitude, different type of immune response, etc.) in the subjects administered the vaccine.

### 3. SUMMARY

Provided herein are, inter alia, compositions (e.g., vaccine booster compositions) comprising a hIL-10R binding agent (e.g., a hIL-10R binding protein or a nucleic acid molecule encoding the same) and in some embodiments an immunogen (e.g., an immunogenic protein or a nucleic acid molecule encoding the same); methods of manufacturing; and pharmaceutical compositions. Further provided herein are methods of utilizing hIL-10R binding agents (e.g., hIL-10R binding proteins or nucleic acid molecules encoding the same), including, e.g., in methods of vaccination (e.g., as vaccine boosters), methods of treating, ameliorating, or preventing infection, methods of promoting an immune response, and methods of increasing mucosal immunogen-specific IgA.

Accordingly, in one aspect, provided herein are combination therapies comprising (a) an immunogenic protein (or an immunogenic fragment and/or immunogenic variant thereof) or a nucleic acid molecule comprising a coding region encoding an immunogenic protein (or an immunogenic fragment and/or immunogenic variant thereof); and (b) a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof).

In some embodiments, the combination therapy is utilized in a vaccine regimen. In some embodiments, the combination therapy is utilized in a prime-boost vaccine regimen. In some embodiments, (a) is utilized as a prime vaccine and (b) is utilized as a boost vaccine of the prime-boost vaccine regimen. In some embodiments, (a) is utilized as a prime vaccine and (b) is utilized as a prime vaccine of the prime-boost vaccine regimen.

In some embodiments, (a) is utilized as a boost vaccine and (b) is utilized as a boost vaccine of the prime-boost vaccine regimen. In some embodiments, (a) and (b) are administered concurrently or sequentially. In some embodiments, (a) is administered prior to (b). In some embodiments, (a) and (b) are not-co-formulated.

In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In some embodiments, hIL-10R binding protein is operably connected to a heterologous moiety either directly or through a linker (e.g., peptide linker). In some embodiments, the heterologous moiety comprises an immunoglobulin Fc region.

In some embodiments, (a) comprises an immunogenic SARS-CoV-2 protein (or an immunogenic fragment and/or immunogenic variant thereof). In some embodiments, (a) comprises a nucleic acid molecule encoding an immunogenic SARS-CoV-2 protein (or an immunogenic fragment and/or immunogenic variant thereof).

In some embodiments, (b) comprises a hIL-10R binding protein (or a functional fragment and/or functional variant thereof). In some embodiments, (b) comprises a nucleic acid molecule comprising a coding region encoding a hIL-10R binding protein (or a functional fragment and/or functional variant thereof).

In some embodiments, the nucleic acid molecule is an RNA molecule. In some embodiments, the RNA molecule is an mRNA molecule or a circular RNA molecule.

In some embodiments, the combination therapy further comprises an IgA inducing protein (IGIP) protein (or a functional fragment and/or functional variant thereof) or a nucleic acid molecule comprising a coding region encoding the IGIP protein (or the functional fragment and/or functional variant thereof).

In some embodiments, the amino acid sequence of the IGIP protein comprises an amino acid sequence at least about 90%, 91%, 92%, 93%, 94%, 95%, 96%, 97%, 98%, 99%, or 100% identical to the amino acid sequence set forth in any one of SEQ ID NOS: 570-572.

In some embodiments, the combination therapy further comprising an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof) that is utilized as part of the boost vaccine of the prime-boost vaccine regimen.

In some embodiments, (a) and/or (b) is formulated in a carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid.

In one aspect, provided herein are vaccine compositions comprising (a) an immunogenic protein (or an immunogenic fragment and/or immunogenic variant thereof) or a nucleic acid molecule comprising a coding region encoding an immunogenic protein (or an immunogenic fragment and/or immunogenic variant thereof); and (b) a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof).

In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In one aspect, provided herein are nucleic acid molecules comprising (a) a coding region encoding a first immunogenic protein (or an immunogenic fragment and/or immunogenic variant thereof) and (b) a coding region encoding a hIL-10R binding protein (or a functional fragment and/or functional variant thereof).

In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178. In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188. In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In one aspect, provided herein are vectors comprising a nucleic acid molecule described herein.

In one aspect, provided herein are carriers comprising a combination therapy described herein, a vaccine composition described herein, a nucleic acid molecule described herein, or a vector described herein.

In one aspect, provided herein are cells comprising a combination therapy described herein, a vaccine composition described herein, a nucleic acid molecule described herein, a vector described herein, or a carrier described herein.

In one aspect, provided herein are pharmaceutical compositions comprising a combination therapy described herein, a vaccine composition described herein, a nucleic acid molecule described herein, a vector described herein, a cell described herein, or a carrier described herein.

In one aspect, provided herein are vaccine compositions comprising a combination therapy described herein, a pharmaceutical composition described herein, a nucleic acid molecule described herein, a vector described herein, a cell described herein, or a carrier described herein.

In one aspect, provided herein are kits comprising a combination therapy described herein, a vaccine composition described herein, a nucleic acid molecule described herein, a vector described herein, a cell described herein, a carrier described herein, or a pharmaceutical composition described herein.

In one aspect, provided herein are methods of vaccinating a subject, the method comprising administering to the subject (a) an immunogenic protein (or an immunogenic fragment and/or immunogenic variant thereof) or a nucleic acid molecule comprising a coding region encoding an immunogenic protein (or an immunogenic fragment and/or immunogenic variant thereof); in combination with (b) a hIL-10R binding protein (or a functional fragment and/or functional variant thereof) or a nucleic acid molecule comprising a coding region encoding a hIL-10R binding protein (or a functional fragment and/or functional variant thereof), to thereby vaccinate the subject.

In one aspect, provided herein are methods of treating a subject exposed to an infective agent, the method comprising administering to the subject (a) an immunogenic protein derived from the infective agent (or an immunogenic fragment and/or immunogenic variant thereof) or a nucleic acid molecule comprising a coding region encoding the immunogenic protein (or the immunogenic fragment and/or immunogenic variant thereof), in combination with (b) a hIL-10R binding protein (or a functional fragment and/or functional variant thereof) or a nucleic acid molecule comprising a coding region encoding a hIL-10R binding protein (or a functional fragment and/or functional variant thereof), to thereby treat the subject.

In one aspect, provided herein are methods of ameliorating, treating, or preventing an infection in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the infection in the subject.

In one aspect, provided herein are methods of ameliorating, treating, or preventing an acute infection in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the acute infection in the subject.

In one aspect, provided herein are methods of ameliorating, treating, or preventing a disease associated with an infection, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the disease associated with the infection in the subject.

In one aspect, provided herein are methods of ameliorating, treating, or preventing severe disease associated with an infection, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the severe disease associated with the infection in the subject.

In one aspect, provided herein are methods of ameliorating, treating, or preventing post viral syndrome, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the post viral syndrome in the subject.

In one aspect, provided herein are methods of enhancing an immunogen-specific immune response in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby enhance the immunogen specific immune response in the subject.

In one aspect, provided herein are methods of increasing the level of immunogen-specific mucosal IgA in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby increasing the level of immunogen-specific mucosal IgA in the subject.

In one aspect, provided herein are methods of increasing the level of immunogen-specific IgG in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby increasing the level of immunogen-specific IgG in the subject.

In one aspect, provided herein are methods of promoting the generation of, enhancing the generation of, and/or sustaining the level of plasma cells in a subject, the method comprising administering to the subject (a) a hIL-10R binding protein (or a functional fragment and/or functional variant thereof) or a nucleic acid molecule comprising a coding region encoding a hIL-10R binding protein (or a functional fragment and/or functional variant thereof), to thereby promote the generation of, enhance the generation of, and/or sustain the level of plasma cells in the subject.

In one aspect, provided herein are methods of ameliorating, reducing, or preventing reactogenicity induced by administration of a vaccine, the method comprising (a) a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, reduce, or prevent reactogenicity induced by administration of the vaccine the subject.

The following embodiments, should be understood to be applicable to any of the foregoing aspects.

In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178.

In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188.

In some embodiments, the amino acid sequence of the encoded hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In some embodiments, the subject has been vaccinated against the infection with at least a first dose of an immunogen.

In some embodiments, the method comprises administering to the subject (b) an immunogenic protein (or an immunogenic fragment and/or immunogenic variant thereof) or a nucleic acid molecule comprising a coding region encoding an immunogenic protein (or an immunogenic fragment and/or immunogenic variant thereof), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect provided herein are combination therapies comprising (a) an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof); and (b) a hIL-10R binding protein (or a functional fragment and/or functional variant thereof) or a nucleic acid molecule comprising a coding region encoding a hIL-10R binding protein (or a functional fragment and/or functional variant thereof).

In some embodiments, the combination therapy is utilized in a vaccine regimen. In some embodiments, the combination therapy is utilized in a prime-boost vaccine regimen. In some embodiments, (a) is utilized as a prime vaccine and (b) is utilized as a boost vaccine of the prime-boost vaccine regimen. In some embodiments, (a) is utilized as a prime vaccine and (b) is utilized as a prime vaccine of the prime-boost vaccine regimen. In some embodiments, (a) is utilized as a boost vaccine and (b) is utilized as a boost vaccine of the prime-boost vaccine regimen.

In some embodiments, (a) and (b) are administered concurrently or sequentially. In some embodiments, (a) is administered prior to (b). In some embodiments, (a) and (b) are not-co-formulated.

In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In some embodiments, hIL-10R binding protein is operably connected to a heterologous moiety either directly or through a linker (e.g., peptide linker). In some embodiments, the heterologous moiety comprises an immunoglobulin Fc region.

In some embodiments, (a) comprises an immunogenic SARS-CoV-2 protein (or an immunogenic fragment and/or immunogenic variant thereof). In some embodiments, (a) comprises a nucleic acid molecule encoding an immunogenic SARS-CoV-2 protein (or an immunogenic fragment and/or immunogenic variant thereof).

In some embodiments, (b) comprises a hIL-10R binding protein (or a functional fragment and/or functional variant thereof). In some embodiments, (b) comprises a nucleic acid molecule comprising a coding region encoding a hIL-10R binding protein (or a functional fragment and/or functional variant thereof).

In some embodiments, the nucleic acid molecule is an RNA molecule. In some embodiments, the RNA molecule is an mRNA molecule or a circular RNA molecule.

In some embodiments, the combination composition further comprises an IgA inducing protein (IGIP) protein (or a functional fragment and/or functional variant thereof) or a nucleic acid molecule comprising a coding region encoding the IGIP protein (or the functional fragment and/or functional variant thereof).

In some embodiments, the amino acid sequence of the IGIP protein comprises an amino acid sequence at least about 90%, 91%, 92%, 93%, 94%, 95%, 96%, 97%, 98%, 99%, or 100% identical to the amino acid sequence set forth in any one of SEQ ID NOS: 570-572.

In some embodiments, the combination composition further comprises an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof) that is utilized as part of the boost vaccine of the prime-boost vaccine regimen.

In some embodiments, (a) and/or (b) is formulated in a carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid.

In one aspect, provided herein are methods of vaccinating a subject comprising administering to the subject in need thereof (a) an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof); in combination with (b) a hIL-10R binding protein (or a functional fragment and/or functional variant thereof) or a nucleic acid molecule comprising a coding region encoding a hIL-10R binding protein (or a functional fragment and/or functional variant thereof), to thereby vaccinate the subject.

In some embodiments, (b) is administered to the subject after (a). In some embodiments, (b) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after (a) is administered to the subject. In some embodiments, (b) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after (a) is administered to the subject. In some embodiments, (b) is administered to the subject about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after (a) is administered to the subject.

In some embodiments, the administering of (a) comprises intramuscular, subcutaneous, or intranasal administration and the administering of (b) comprises intramuscular, subcutaneous, or intranasal administration. In some embodiments, the administering of (a) comprises intramuscular or subcutaneous administration and the t administering of (b) comprises intramuscular or subcutaneous administration. In some embodiments, the administering of (a) comprises intramuscular or subcutaneous administration and the administering of (b) comprises intranasal administration. In some embodiments, the administering of (a) comprises intranasal administration and the administering of (b) comprises intranasal administration.

In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188. In some embodiments, the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

In some embodiments, the hIL-10R binding protein is operably connected to a heterologous moiety either directly or through a linker (e.g., peptide linker). In some embodiments, the heterologous moiety comprises an immunoglobulin Fc region.

In some embodiments, (a) comprises an immunogenic SARS-CoV-2 protein (or an immunogenic fragment and/or immunogenic variant thereof). In some embodiments, (a) comprises a nucleic acid molecule encoding an immunogenic SARS-CoV-2 protein (or an immunogenic fragment and/or immunogenic variant thereof).

In some embodiments, (b) comprises a hIL-10R binding protein (or a functional fragment and/or functional variant thereof). In some embodiments, (b) comprises a nucleic acid molecule comprising a coding region encoding a hIL-10R binding protein (or a functional fragment and/or functional variant thereof).

In some embodiments, the nucleic acid molecule is an RNA molecule. In some embodiments, the RNA molecule is an mRNA molecule or a circular RNA molecule.

In some embodiments, the method further comprises administering an IgA inducing protein (IGIP) protein (or a functional fragment and/or functional variant thereof) or a nucleic acid molecule comprising a coding region encoding the IGIP protein (or the functional fragment and/or functional variant thereof) to the subject.

In some embodiments, the amino acid sequence of the IGIP protein comprises an amino acid sequence at least about 90%, 91%, 92%, 93%, 94%, 95%, 96%, 97%, 98%, 99%, or 100% identical to the amino acid sequence set forth in any one of SEQ ID NOS: 570-572.

In some embodiments, the method further comprises administering an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof) concurrently with the hIL-10R binding protein (or a functional fragment and/or functional variant thereof) or the nucleic acid molecule comprising a coding region encoding a hIL-10R binding protein (or a functional fragment and/or functional variant thereof).

In some embodiments, (a) and/or (b) is formulated in a carrier. In some embodiments, the carrier is a lipid nanoparticle (LNP), liposome, lipoplex, or nanoliposome. In some embodiments, the carrier is an LNP. In some embodiments, wherein the LNP comprises a cationic lipid, a neutral lipid, a cholesterol, and/or a PEG lipid.

### 4. BRIEF DESCRIPTION OF THE FIGURES

FIGS. 1A-1C are line graphs showing the IgM, IgG1, or IgA production in human PBMCs (hPBMCs) treated with the indicated hIL-10R binding protein hIL-10R BFP-1 (FIG. 1A), hIL-10R BFP-2 (FIG. 1B), or hIL-10R BFP-10 (FIG. 1C). Fc-GFP was utilized as a control.

FIGS. 2A-2B are bar graphs showing the anti-Spike S1 IgG (FIG. 2A) and anti-nucleocapsid IgG (FIG. 2B) production from hPBMCs (obtained from a donor known to have been previously administered a SARS-COV-2 vaccine (Vaccinated) or a donor with an unknown SARS-COV-2 vaccination status (Unknown) treated with the indicated hIL-10R binding protein hIL-10R BFP-1, hIL-10R BFP-2, or hIL-10R BFP-10; and a SARS-Cov-2 spike protein. Fc-GFP was utilized as a control. The fold increase in antibody production relative to hIL-10R BFP-1 is indicated.

FIGS. 3A-3B are bar graphs showing the anti-Spike S1 IgG (FIG. 3A) and the anti-Spike S1 IgA (FIG. 3B) production from hPBMCs (obtained from a donor known to have been previously administered a SARS-COV-2 vaccine (Vaccinated) or a donor with an unknown SARS-COV-2 vaccination status (Unknown) treated with the indicated hIL-10R binding protein hIL-10R BFP-1, hIL-10R BFP-2, or hIL-10R BFP-10; and CD40 ligand (CD40L). Fc-GFP was utilized as a control. Fold change increase in antibody production relative to hIL-10R BFP-1 is indicated.

FIG. 4 is a bar graph showing the level of plasmablasts (as a percentage of total B linage cells) in PBMCs treated with the indicated hIL-10R binding protein hIL-10R BFP-1, hIL-10R BFP-2, or hIL-10R BFP-10 (or control (untreated, Fc)) in the absence (Unstimulated) or presence (PrepTivator Spike) of antigen.

FIG. 5 is a graph showing the expression (by mean fluorescence intensity (X-axis)) of the hIL-10Rα subunit by each of the indicated immune cell populations (CD14+ monocytes, B Cells, CD4+ T cells, and CD8+ T cells). Each point represents the mean of 3 replicate hPBMC samples from a different donor. Data has been normalized by subtracting the MFI of the FMO negative control from each sample.

FIG. 6 is a graph showing the expression (by mean fluorescence intensity (X-axis)) of the hIL-10Rβ subunit by each of the indicated immune cell populations (CD14+ monocytes, B Cells, CD4+ T cells, and CD8+ T cells). Each point represents the mean of 3 replicate hPBMC samples from a different donor. Data has been normalized by subtracting the MFI of the FMO negative control from each sample.

FIG. 7 is a graph showing the relative binding affinity of hIL-10R BFP-1 and hIL-10R BFP-10 for the hIL-10Rα chain (Y axis) and the hIL-10Rβ chain (X axis).

FIG. 8 is a bar graph showing the expression of antigen (SARS-CoV-2 spike protein) specific IgG antibodies (left bar of each control or treatment group) and antigen (SARS-CoV-2 spike protein) specific IgA antibodies (right bar of each control or treatment group) for each of the indicated treatment groups (Fc-GFP; hIL-10R BFP-1; and hIL-10R BFP-10).

FIG. 9 is a FACS plot showing the percentage of mature plasma cells and other B cells in unstimulated B cell populations (left plot) and antigen (SARS-CoV-2 spike protein) stimulated B cell populations.

FIG. 10 is a bar graph showing the expression of IFNγ (pg/mL) by antigen (SARS-CoV-2 spike protein) stimulated T cells treated with the indicated control (untreated, Fc-GFP) or agent (hIL-10R BFP-1 or hIL-10R BFP-10).

FIG. 11 is a bar graph showing the expression of IL-6 (pg/mL) by LPS stimulated monocytes treated with the indicated control (untreated) or agent (hIL-10R BFP-1 or hIL-10R BFP-10).

FIG. 12 is a bar graph showing the expression of IL-1β (pg/mL) by LPS stimulated monocytes treated with the indicated control (untreated) or agent (hIL-10R BFP-1 or hIL-10R BFP-10).

### 5. DETAILED DESCRIPTION

Some vaccines, while capable of limiting the severity of disease associated with an infection, may be less effective at preventing infection, for any of a number of reasons. For example, a vaccine may not generate an immune response of sufficient magnitude to prevent an infection from taking hold, may not induce an immune response of sufficient length, may not generate a specific type of immune response (e.g., B cell response, T cell response), and/or may not generate a sufficient immune response in a particular compartment of the body (e.g., the tissue that first comes in contact with an infective agent). For example, SARS-CoV-2-specific nasal IgA may not be induced by vaccination (see, e.g., Liew et al., EBioMedicine (2023) 87:104402, the entire contents of which is incorporated by reference herein for all purposes).

The inventors have, inter alia, discovered that an hIL-10R agonist can enhance a subject's immune response to an immunogen (e.g., can increase immunogen-specific IgA) when administered in a combination regimen with the immunogen. An hIL-10R agonist can be an agonistic hIL-10R binding agent (e.g., hIL-10R binding protein, a nucleic acid molecule encoding an hIL-10R binding protein, or an agonist hIL-10R binding small molecule). As such, the disclosure provides, inter alia, compositions (e.g., vaccine compositions) comprising a hIL-10R binding agent (e.g., an agonist hIL-10R binding small molecule, a hIL-10R binding protein or a nucleic acid molecule encoding the same) and methods of using the same, e.g., methods of vaccination, methods of preventing or treating an infection, methods of enhancing an immune response, and methods of increasing immunogen-specific IgA, e.g., mucosal IgA.

Table of Contents

| | |
|---|---|
| 5.1 | Definitions |
| 5.2 | hIL-10 Receptor Binding Agents |
| 5.3 | Potency & Affinity of hIL-10R Binding Agents |
| 5.4 | Nucleic Acid Molecules Encoding hIL-10R Binding Proteins |
| 5.4.1 | DNA Molecules |
| 5.4.2 | RNA Molecules |
| 5.5 | Immunogens |
| 5.6 | Nucleic Acid Molecules Encoding Immunogens |
| 5.6.1 | DNA Molecules |
| 5.6.2 | RNA Nucleic Acids |
| 5.7 | IgA Inducing Protein (IGIP) |
| 5.8 | Nucleic Acid Molecules Encoding IGIP Proteins |
| 5.8.1 | DNA Molecules |
| 5.8.2 | RNA Molecules |
| 5.9 | Signal Peptides |
| 5.10 | Fusions & Conjugates |
| 5.10.1 | Ig Fusion Proteins |
| 5.10.1.1 | Ig Effector Function |
| 5.10.2 | Linkers |
| 5.10.3 | Orientation |
| 5.10.4 | Multimeric Fusion Proteins |
| 5.10.5 | Exemplary hIL-10R Binding Protein - Ig Fusion Proteins & Polypeptides |
| 5.11 | Polycistronic Nucleic Acid Molecules |
| 5.11.1 | Plurality of Immunogens |
| 5.12 | Combination Compositions |
| 5.12.1 | Nucleic Acid-Based Compositions |
| 5.12.1.1 | Plurality of Immunogens |
| 5.12.2 | Protein-Based Compositions |
| 5.12.2.1 | Plurality of Immunogens |
| 5.13 | Vaccine Compositions |
| 5.13.1 | Vaccine Prime Compositions |
| 5.13.1.1 | Protein-Based Vaccine Prime Compositions |
| 5.13.1.1 (i) | Plurality of Immunogens |
| 5.13.1.2 | Nucleic Acid-Based Vaccine Prime Compositions |
| 5.13.1.2 (i) | Plurality of Immunogens |
| 5.13.1.3 | Vaccine Prime Formulation for Administration |
| 5.13.2 | Vaccine Booster Compositions |
| 5.13.2.1 | Protein-Based Vaccine Booster Compositions |
| 5.13.2.1 (i) | Plurality of Immunogens |
| 5.13.2.2 | Nucleic Acid-Based Vaccine Booster Compositions |
| 5.13.2.2 (i) | Plurality of Immunogens |
| 5.13.2.3 | Vaccine Booster Formulation for Administration |
| 5.13.3 | Combinations Therapies |
| 5.13.3.1 | Exemplary Combinations of Vaccine Prime and Booster Compositions |
| 5.14 | Vectors |
| 5.15 | Carriers |
| 5.15.1 | Lipid Based Carriers/Lipid Nanoformulations |
| 5.15.1.1 | Cationic Lipids (Positively Charged) and Ionizable Lipids |
| 5.15.1.2 | Non-Cationic Lipids (e.g., Phospholipids) |
| 5.15.1.3 | Structural Lipids |
| 5.12.1.4 | Polymers and Polyethylene Glycol (PEG) - Lipids |
| 5.15.1.5 | Percentages of Lipid Nanoformulation Components |
| 5.16 | Methods of Making Proteins |
| 5.17 | Methods of Making Nucleic Acid Molecules |
| 5.18 | Nucleic Acid Molecules, Vectors, Host Cells, & Carriers |
| 5.19 | Adjuvants |
| 5.20 | Pharmaceutical Compositions |
| 5.21 | Methods of Use |
| 5.21.1 | Methods of Vaccination |
| 5.21.1.1 | Methods of Vaccinating a Subject |
| 5.21.1.2 | Methods of Vaccinating a Subject Utilizing an mRNA Vaccine |
| 5.21.1.3 | Methods of Vaccinating a Subject Against SARS-CoV-2 |
| 5.21.2 | Methods of Ameliorating, Treating, or Preventing Infections |
| 5.21.2.1 | Methods of Ameliorating, Treating, or Preventing an Infection |
| 5.21.2.2 | Methods of Ameliorating, Treating, or Preventing an Infection in Vulnerable |
| | Sub-Populations of Subjects |
| 5.21.2.3 | Methods of Ameliorating, Treating, or Preventing an Acute Infection |
| 5.21.3 | Methods of Ameliorating, Treating, or Preventing Infection Associated |
| | Disease |
| 5.21.3.1 | Methods of Ameliorating, Treating, or Preventing Severe Disease Associated |
| | with an Infection |
| 5.21.3.2 | Methods of Ameliorating, Treating, or Preventing Post Viral Syndrome |
| 5.21.4 | Methods of Enhancing an Immunogen-Specific Immune Response |
| 5.21.5 | Methods of Increasing the Level of Immunogen-Specific Mucosal IgA |
| 5.21.6 | Methods of Increasing the Level of Immunogen-Specific IgG |
| 5.21.7 | Methods of Promoting, Enhancing, and/or Sustaining Plasma Cell |
| | Populations |
| 5.21.8 | Methods of Modulating (e.g., Preventing, Ameliorating, Reducing) Vaccine |
| | Reactogenicity |
| 5.22 | Kits |
| 5.23 | Exemplary Embodiments |
| 5.1 | Definitions |

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

Where proteins and/or polypeptides are described herein, it is understood that nucleic acid molecules (e.g., RNA (e.g., mRNA) or DNA nucleic acid molecules) encoding the protein or polypeptide are also provided herein.

Where proteins, polypeptides, nucleic acid molecules, vectors, carriers, etc. are described herein, it is understood that isolated forms of the proteins, polypeptides, nucleic acid molecules, vectors, carriers, etc. are also provided herein.

Where proteins, polypeptides, nucleic acid molecules, etc. are described herein, it is understood that recombinant forms of the proteins, polypeptides, nucleic acid molecules, etc. are also provided herein.

Where proteins or sets of proteins are described herein, it is understood that both proteins comprising the primary structure are provided herein as well as proteins folded into their three-dimensional structure (i.e., tertiary or quaternary structure) are provided herein.

Where proteins are described herein, it is understood that functional variants, functional fragment, and functional variants and fragments are provided herein. It is understood that the terms “functional fragment or variant,” “functional fragment or functional variant,” “functional fragment and/or functional variant” and the like—provide specific disclosure of proteins that are functional fragments, functional variants, and functional fragments and functional variants (of the reference protein).

As used herein, the term “acute COVID” refers to the signs and symptoms of COVID-19 which last for up to about 4 weeks after initial infection with SARS-CoV-2.

As used herein, the term “adjuvant” refers to a substance that stimulates the immune system of a subject when administered to the subject.

As used herein, the term “administering” refers to the physical introduction of an agent, e.g., a therapeutic agent (or a precursor of the therapeutic agent that is metabolized or altered within the body of the subject to produce the therapeutic agent in vivo) (e.g., a vaccine) to a subject, using any of the various methods and delivery systems known to those skilled in the art. Administering can also be performed, for example, once, a plurality of times, and/or over one or more extended periods. Therapeutic agents include agents whose effect is intended to be preventative (i.e., prophylactic), such as vaccine compositions (e.g., vaccine prime compositions, vaccine booster compositions).

As used herein, the term “affinity” refers to the strength of the binding of one protein (e.g., a Ligand) to another protein (e.g., a Receptor). The affinity of a protein is measured by the dissociation constant Kd, defined as [Ligand]×[Receptor]/[Ligand-Receptor] where [Ligand-Receptor] is the molar concentration of the Ligand-Receptor complex, [Ligand] is the molar concentration of the unbound Ligand and [Receptor] is the molar concentration of the unbound Receptor. The affinity constant Ka is defined by 1/Kd. Standard methods of measuring affinity are known to the person of ordinary skill in the art.

As used herein, the term “agent” is used generically to describe any macro or micro molecule. Exemplary molecules include, but are not limited to polypeptides, proteins, peptides, nucleic acid molecules (e.g., DNA molecules, RNA molecules), small molecules, carbohydrates, lipids, synthetic polymers (e.g., polymers of PEG).

As used herein, the term “antibody” or “antibodies” is used in the broadest sense and encompasses various immunoglobulin (Ig) (e.g., human Ig (hIg), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂—Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In certain embodiments, antibodies described herein are mIgG antibodies, or a class (e.g., mIgG1 or mIgG2a) or subclass thereof. In some embodiments, the term antibodies refers to a monoclonal or polyclonal antibody population. Antibodies described herein can be produced by any standard methods known in the art, e.g., recombinant production in host cells, see, e.g., § 5.16; or synthetic production.

As used herein, the term “circular RNA” refers to a translatable RNA molecule that forms a circular structure through covalent or non-covalent bonds. In some embodiments, the RNA molecule forms a circular structure through covalent bonds.

As used herein, the term “conjugation” refers to chemical conjugation of a protein with a moiety (e.g., small molecule, polypeptide, polynucleotide, carbohydrate, lipid, synthetic polymer (e.g., polymers of polyethylene glycol (PEG)), etc.). The moiety can be directly connected to the protein or indirectly connected through a linker, e.g., as described herein. Chemical conjugation methods are well known in the art, as are commercially available conjugation reagents and kits, with detailed instructions for their use readily available from the commercial suppliers.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the polynucleotide, polypeptide, or protein. For example, the polynucleotide, polypeptide, or protein can be recombinantly produced or chemically synthesized.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology. The term disease includes infection (e.g., a viral, bacterial, fungal, protozoal infection).

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “EC50” or “half maximal effective concentration” is a measure of potency of an agent (e.g., a hIL-10R binding protein described herein) and refers to the concentration of the agent (e.g., a hIL-10R binding protein described herein) required to induce a response halfway between baseline and maximal response after a particular exposure period. Assays to measure the EC50 of a protein are standard in the art, see, also, e.g., § 5.3.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and Clq binding.

As used herein, the term “Fe region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region, see, e.g., § 5.10.1.1. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

The term “functional variant” as used herein in reference to a protein refers to a protein that comprises at least one but no more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein. For example, a functional variant of a hIL-10 protein can refer to a hIL-10 protein comprising one or more amino acid substitution as compared to a reference hIL-10 protein (e.g., wild type) that retains the ability to specifically bind the hIL-10R.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. For example, a functional fragment of hIL-10 can refer to a fragment of hIL-10 that retains the ability to specifically bind the hIL-10R.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to a protein (the subject protein), increases the half-life of the subject protein in vivo when administered to a subject (e.g., a human subject). The pharmacokinetic properties of the protein can be evaluated utilizing in vivo models known in the art.

As used herein, the term “half-life extension polypeptide” refers to a protein that when operably connected to another protein (the subject protein), increases the half-life of the subject protein in vivo when administered to a subject (e.g., a human subject). The pharmacokinetic properties of the protein can be evaluated utilizing in vivo models known in the art.

As used herein, the term “heterologous,” when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, polynucleotide, carbohydrate, lipid, synthetic polymer (e.g., polymers of PEG), etc.) that is not joined to the polypeptide in nature.

As used, herein the term “heterologous signal peptide” refers to a signal peptide that is not operably connected to a subject protein in nature. For example, in reference to a polypeptide comprising a signal peptide from human IL-2 operably connected to human IL-12, the human IL-2 signal peptide would constitute a heterologous signal peptide.

As used herein, the term “homologous signal peptide” refers to a signal peptide that is operably connected to a subject protein in nature. For example, in reference to a polypeptide comprising a signal peptide from human IL-2 operably connected to human IL-2, the human IL-2 signal peptide would constitute a homologous signal peptide.

As used herein, the term “human interleukin 10” or “hIL-10” refers to the human immunomodulatory cytokine that mediates signaling through the human IL-10 Receptor. The amino acid sequence of an exemplary reference mature hIL-10 protein is set forth in SEQ ID NO: 1.

As used herein, the term “human IL-10 Receptor” or “hIL-10R” refers to the human heterodimeric cell surface complex comprised of hIL-10Rα and hIL-10Rβ, through which hIL-10 mediates signaling.

As used herein, the term “human IL-10 Receptor binding agent” or “hIL-10R binding agent” refers to an agent that specifically binds at least one subunit of the hIL-10R: hIL-10Rα and/or hIL-10Rβ. In some embodiments, the hIL-10R binding agent specifically binds hIL-10Rα. In some embodiments, the hIL-10R binding agent specifically binds hIL-10Rβ. In some embodiments, the hIL-10R binding agent specifically binds hIL-10Rα and hIL-10Rβ. In some embodiments, the hIL-10R binding agent specifically binds hIL-10Rα and hIL-10Rβ, and binds hIL-10Rβ with less affinity relative to the affinity for hIL-10Rα. In some embodiments, the hIL-10R binding agent specifically binds hIL-10Rα and hIL-10Rβ, and binds hIL-10Rβ with higher affinity relative to the affinity for hIL-10Rα.

As used herein, the term “human IL-10 Receptor binding protein” or “hIL-10R binding protein” refers to a protein that specifically binds at least one subunit of the hIL-10R: hIL-10Rα and/or hIL-10Rβ. In some embodiments, the hIL-10R binding protein specifically binds hIL-10Rα. In some embodiments, the hIL-10R binding protein specifically binds hIL-10Rβ. In some embodiments, the hIL-10R binding protein specifically binds hIL-10Rα and hIL-10Rβ. In some embodiments, the hIL-10R binding protein specifically binds hIL-10Rα and hIL-10Rβ, and binds hIL-10Rβ with less affinity relative to the affinity for hIL-10Rα. In some embodiments, the hIL-10R binding protein specifically binds hIL-10Rα and hIL-10Rβ, and binds hIL-10Rβ with higher affinity relative to the affinity for hIL-10Rα.

As used herein, the term “human IL-10 Receptor α” or “hIL-10Rα” refers to the alpha (a) subunit of the hIL-10 Receptor. The amino acid sequence of an exemplary reference mature hIL-10Rα polypeptide is set forth in SEQ ID NO: 355.

As used herein, the term “human IL-10 Receptor β” or “hIL-10Rβ” refers to the beta (p) subunit of the hIL-10 Receptor. The amino acid sequence of an exemplary reference mature hIL-10Rβ polypeptide is set forth in SEQ ID NO: 357.

As used herein, the term “IgA inducing protein” or “IGIP” refers to the secreted protein produced by e.g., dendritic cells, that functions, inter alia, in the induction of IgA expression. The amino acid sequence of a first exemplary reference mature human IGIP (hIGIP) protein is set forth in SEQ ID NO: 572. The term IGIP includes naturally occurring and non-naturally occurring variants of IGIP.

As used herein, the term “immunogen” refers to a substance that is capable of inducing an immune response (e.g., an adaptive immune response) in a subject (e.g., a human). An immunogen may have one or more isoforms, sequence variants, or splice variants that have equivalent biological and immunological activity, and are thus also considered for the purposes of this disclosure to be immunogenic equivalents of the immunogen.

As used herein, the term “immunogenic protein” refers to a protein that comprises an immunogen.

As used herein, the term “in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen (e.g., a prime-boost vaccine regimen). In some embodiments, administration of two or more agents or treatments in combination is such that the reduction in a symptom, or other parameter related to the condition is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first agent of the combination may be administered by intramuscular injection while a second agent of the combination may be administered intranasally.

As used herein, the term “isolated” with reference to a polypeptide, protein, or polynucleotide refers to a polypeptide, protein, or polynucleotide that is substantially free of other cellular components with which it is associated in the natural state.

As used herein, the term “long COVID” is commonly used to refer to signs and symptoms that continue or develop after acute COVID-19. Long COVID is also referred to in the art as persistent post-Covid syndrome (PPCS), post-acute sequelae of COVID-19 (PASC), long haul COVID, and chronic COVID. The term long COVID encompasses any clinically acceptable definition.

As used herein, the term “moiety” is used generically to describe any macro or micro molecule that can be operably connected to a protein described herein. Exemplary moieties include, but are not limited small molecules, polypeptides, polynucleotides (e.g., DNA, RNA), carbohydrates, lipids, synthetic polymers (e.g., polymers of PEG).

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

The determination of “percent identity” between two sequences (e.g., protein (amino acid sequences) or polynucleotide (nucleic acid sequences)) can be accomplished using a mathematical algorithm. The determination of percent identity between two sequences is a common method known to the those of ordinary skill in the art. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “poly(A) sequence,” refers to a sequence of adenosine nucleotides. A poly(A) is typically located at the 3′-end of a coding linear RNA (e.g., an mRNA). In some embodiments, the poly(A) comprises up to about 1000 adenosine nucleotides. In some embodiments, the poly(A) sequence is essentially homopolymeric, e.g., apoly(A) sequence of e.g., 100 adenosine nucleotides having essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to mRNA—however in the context of the invention, the term likewise relates to corresponding sequences in a DNA molecule (e.g., a “poly(T) sequence”).

As used herein, the term, “polycistronic” with reference to a nucleic acid molecule refers to a nucleic acid molecule (e.g., DNA, RNA) that comprises more than one coding region encoding a protein. For example, a polycistronic nucleic acid molecule (e.g., DNA, RNA) may comprise a first coding region encoding a first protein and a second coding region encoding a second protein, wherein the first protein is different from the second protein.

As used herein, the term, “prime-boost” with reference to a vaccine regimen refers to a vaccine regimen comprising a first administration of an immunogen to a subject (the vaccine prime) and sometime thereafter administration of a vaccine booster.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “plurality” means 2 or more (e.g., 3 or more, 4 or more, 5 or more, 6 or more, 7 or more, 9 or more, or 10 or more).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where polypeptides are contemplated herein, it should be understood that proteins folded into their three-dimensional structure are also provided herein.

A “prophylactic” treatment is a treatment administered to a subject who does not exhibit signs of a disease or exhibits only early signs for the purpose of decreasing the risk of developing pathology.

As used herein, the term “reactogenicity” refers to symptoms that are generally associated with an inflammatory response to a vaccination. The symptoms can be divided into both local symptoms (e.g., pain, swelling, and/or erythema at the site of administration of the vaccine (e.g., site of injection)) and systemic symptoms (e.g., fever, nausea, vomiting, diarrhea, headaches, fatigue, and/or myalgia).

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “scFv” refers to an antibody that comprises a VL operably connected to a VH (e.g., via a peptide linker). In some embodiments, the VH and VL are operably connected by a peptide linker. The VL and VL can be operably connected in any order (e.g., from N- to C-terminus: VH-optional peptide linker-VL; or N- to C-terminus: VL-optional peptide linker-VH.

As used herein, the term “(scFv)₂” refers to an antibody that comprises a first and a second scFv operably connected (e.g., via a peptide linker). The first and second scFv can specifically bind the same or different antigens. In some embodiments, the first and second scFv are operably connected by a peptide linker.

As used herein, the term “scFv-Fc” refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

As used herein, the term “(scFv)₂-Fc” herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human IL-10 may be cross reactive with IL-10 of another species (e.g., cynomolgus, murine, etc.), and still be considered herein to specifically bind human IL-10. A protein can specifically bind more than one different protein.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the term “translatable RNA” refers to any RNA that encodes at least one polypeptide and can be translated to produce the encoded protein in vitro, in vivo, in situ or ex vivo. A translatable RNA may be an mRNA or a circular RNA encoding a polypeptide.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease (e.g., an infection) does not require that the disease (e.g., an infection), or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease (e.g., an infection) and/or adverse symptom attributable to the disease (e.g., an infection). In some embodiments, the effect is preventative, i.e., the effect protects or prevents an occurrence or reoccurrence of a disease (e.g., an infection).

As used herein, the term “tumor associated immunogen” refers to an immunogen that is either unique to cancer cells and does not occur on other cells in the body of a subject (a cancer specific immunogen) or an immunogen that not unique to a cancer cell and instead is also expressed on a normal (e.g., non-cancer cell) but is overexpressed by a cancer cell in comparison to a normal cell (e.g., a non-cancer cell), for example, 1-fold over expression, 2-fold overexpression, 3-fold overexpression or more in comparison to a normal cell (e.g., non-cancer cell). In some embodiments, the tumor associated immunogen is inappropriately synthesized by the cancer cell, for example, a protein that contains amino acid variations (e.g., amino acid deletions, additions, and/or substitutions), in comparison to the protein expressed by a normal cell (e.g., a non-cancer cell). In some embodiments, the tumor associated immunogen is only expressed by the cancer cell and not expressed at detectable level by normal cells. Methods to identify and verify tumor-associated proteins are known to a skilled person and described in the literature (see, e.g., Bornstein, AAPS J. (2015), vol. 17(3), p. 525-534; Hong et al., BMC Syst Biol. (2018), vol. 12 (Suppl 2), p. 17, the entire contents of which is incorporated by reference herein for all purposes.

As used herein, the term “vaccinated subject” refers to a subject that has received at least one dose of a vaccine regimen. The term includes subjects that are partially vaccinated (i.e., subjects who have received at least one dose of a multi-dose vaccine regimen) or fully vaccinated (i.e., subjects who have received all doses of a vaccine regimen (e.g., single or multi-dose vaccine regimens)).

As used herein, the term “vaccine booster” or “booster” with reference to a vaccine refers to a composition administered after an initial administration of a dose of a first immunogen to a subject that comprises an adjuvant and/or a second dose of a second immunogen. Therefore, vaccine boosters described herein include compositions comprising an adjuvant alone, (e.g., a hIL-10R binding protein described herein (or a nucleic acid molecule encoding the same)), an immunogenic protein (or nucleic acid molecule encoding the same) alone, or a combination of an adjuvant and an immunogenic protein (or nucleic acid molecule encoding the same). The first and second immunogens can be the same or different.

As used herein, the term “variant” or “variation” with reference to a nucleic acid molecule, refers to a nucleic acid molecule that comprises at least one substitution, alteration, inversion, addition, or deletion of nucleotide compared to a reference nucleic acid molecule. As used herein, the term “variant” or “variation” with reference to a peptide or protein refers to a peptide or protein that comprises at least one substitution, alteration, inversion, addition, or deletion of an amino acid residue compared to a reference peptide or protein.

The terms “VL” and “VL domain” are used interchangeably to refer to the light chain variable region of an antibody.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂—Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

As used herein, the term “5′-untranslated region” or “5′-UTR” refers to a part of a nucleic acid molecule located 5′ (i.e., “upstream”) of a coding sequence and which is not translated into protein. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified or varied, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 hIL-10 Receptor Binding Agents

In some aspects described herein, a hIL-10R binding agent (e.g., a hIL-10R binding protein (or a functional fragment and/or functional variant thereof) or a nucleic acid molecule encoding the hIL-10R binding protein (or the functional fragment and/or functional variant thereof), see, e.g., § 5.4) is utilized (e.g., in compositions described herein (see, e.g., §§ 5.12, 5.13, 5.20), in nucleic acid molecules described herein (see, e.g., § 5.11, 5.18), in vaccines described herein (see, e.g., § 5.13), in pharmaceutical compositions described herein (see, e.g., § 5.20), in methods described herein (see, e.g., § 5.21), in kits described herein (see, e.g., § 5.22), etc. In some embodiments, a hIL-10R binding protein (or a functional fragment and/or functional variant thereof) (e.g., described herein) is utilized. In some embodiments, a nucleic acid molecule encoding the hIL-10R binding protein (or a functional fragment and/or functional variant thereof) (e.g., described herein) is utilized.

The hIL-10R mediates cellular responses induced by binding of hIL-10. The hIL-10R comprises two unique subunits, a hIL-10Rα subunit and a hIL-10Rβ subunit. While similar in overall architecture, hIL-10 exhibits lower affinity for hIL-10Rβ relative to the a subunit. hIL-10 is the founding member of the IL-10 cytokine family, which includes IL-19, IL-20, IL-22, IL-24, and IL-26. IL-10 is an important immunoregulatory cytokine and pleiotropic in nature. IL-10 is known to function in part, to suppress inflammatory immune responses and potently inhibit the production of pro-inflammatory cytokines such as IFN-γ, TNFα, IL-1β, and IL-6. IL-10 is further known to prevent dendritic cell maturation in part by inhibiting the expression of IL-12 and the expression of MHC and co-stimulatory molecules important for cell-mediated immunity. IL-10 is also known to mediate pro-inflammatory effects, including the stimulation of IFN-γ and granzyme B production by CD8+ T cells. IL-10 has also been shown to induce IgA (and IgG) production from activated B cells and stimulate differentiation of resting B cells into long-lasting plasma cells.

The amino acid sequence of a reference immature hIL-10 protein and mature hIL-10 protein is set forth in SEQ ID NOS: 1 and 179, respectively. The amino acid sequence of a reference immature hIL-10Rα protein and mature hIL-10Rα protein is set forth in SEQ ID NOS: 354 and 355, respectively. The amino acid sequence of a reference immature hIL-10Rβ protein and mature hIL-10Rβ protein is set forth in SEQ ID NOS: 356 and 357, respectively. See Table 1, herein.

TABLE 1
The Amino Acid Sequence of Reference
hIL-10, hIL-10Rα, and hIL-10Rβ Polypeptides.

| | | SEQ |
|---|---|---|
| Description | Amino Acid Sequence | ID NO |
| hIL-10 | MHSSALLCCLVLLTGVRASPGQGTQSENSCTHFPGNLP | 1 |
| Immature-Signal | NMLRDLRDAFSRVKTFFQMKDQLDNLLLKESLLEDFKG | |
| Peptide Underlined | YLGCQALSEMIQFYLEEVMPQAENQDPDIKAHVNSLGE | |
| | NLKTLRLRLRRCHRFLPCENKSKAVEQVKNAFNKLQEK | |
| | GIYKAMSEFDIFINYIEAYMTMKIRN | |
| hIL-10 | SPGQGTQSENSCTHFPGNLPNMLRDLRDAFSRVKTFFQ | 179 |
| Mature-No Signal | MKDQLDNLLLKESLLEDFKGYLGCQALSEMIQFYLEEV | |
| Peptide | MPQAENQDPDIKAHVNSLGENLKTLRLRLRRCHRFLPC | |
| | ENKSKAVEQVKNAFNKLQEKGIYKAMSEFDIFINYIEA | |
| | YMTMKIRN | |
| hIL-10Rα | MLPCLVVLLAALLSLRLGSDAHGTELPSPPSVWFEAEF | 354 |
| Immature-Signal | FHHILHWTPIPNQSESTCYEVALLRYGIESWNSISNCS | |
| Peptide Underlined | QTLSYDLTAVTLDLYHSNGYRARVRAVDGSRHSNWTVT | |
| | NTRFSVDEVTLTVGSVNLEIHNGFILGKIQLPRPKMAP | |
| | ANDTYESIFSHFREYEIAIRKVPGNFTFTHKKVKHENF | |
| | SLLTSGEVGEFCVQVKPSVASRSNKGMWSKEECISLTR | |
| | QYFTVINVIIFFAFVLLLSGALAYCLALQLYVRRRKKL | |
| | PSVLLFKKPSPFIFISQRPSPETQDTIHPLDEEAFLKV | |
| | SPELKNLDLHGSTDSGFGSTKPSLQTEEPQFLLPDPHP | |
| | QADRTLGNREPPVLGDSCSSGSSNSTDSGICLQEPSLS | |
| | PSTGPTWEQQVGSNSRGQDDSGIDLVQNSEGRAGDTQG | |
| | GSALGHHSPPEPEVPGEEDPAAVAFQGYLRQTRCAEKA | |
| | TKTGCLEEESPLTDGLGPKFGRCLVDEAGLHPPALAKG | |
| | YLKQDPLEMTLASSGAPTGQWNQPTEEWSLLALSSCSD | |
| | LGISDWSFAHDLAPLGCVAAPGGLLGSFNSDLVTLPLI | |
| | SSLQSSE | |
| hIL-10Rα | HGTELPSPPSVWFEAEFFHHILHWTPIPNQSESTCYEV | 355 |
| Mature-No Signal | ALLRYGIESWNSISNCSQTLSYDLTAVTLDLYHSNGYR | |
| Peptide | ARVRAVDGSRHSNWTVTNTRFSVDEVTLTVGSVNLEIH | |
| | NGFILGKIQLPRPKMAPANDTYESIFSHFREYEIAIRK | |
| | VPGNFTFTHKKVKHENFSLLTSGEVGEFCVQVKPSVAS | |
| | RSNKGMWSKEECISLTRQYFTVTNVIIFFAFVLLLSGA | |
| | LAYCLALQLYVRRRKKLPSVLLFKKPSPFIFISQRPSP | |
| | ETQDTIHPLDEEAFLKVSPELKNLDLHGSTDSGFGSTK | |
| | PSLQTEEPQFLLPDPHPQADRTLGNREPPVLGDSCSSG | |
| | SSNSTDSGICLQEPSLSPSTGPTWEQQVGSNSRGQDDS | |
| | GIDLVQNSEGRAGDTQGGSALGHHSPPEPEVPGEEDPA | |
| | AVAFQGYLRQTRCAEKATKTGCLEEESPLTDGLGPKFG | |
| | RCLVDEAGLHPPALAKGYLKQDPLEMTLASSGAPTGQW | |
| | NQPTEEWSLLALSSCSDLGISDWSFAHDLAPLGCVAAP | |
| | GGLLGSFNSDLVTLPLISSLQSSE | |
| hIL-10Rβ | MAWSLGSWLGGCLLVSALGMVPPPENVRMNSVNFKNIL | 356 |
| Immature-Signal | QWESPAFAKGNLTFTAQYLSYRIFQDKCMNTTLTECDF | |
| Peptide Underlined | SSLSKYGDHTLRVRAEFADEHSDWVNITFCPVDDTIIG | |
| | PPGMQVEVLADSLHMRFLAPKIENEYETWTMKNVYNSW | |
| | TYNVQYWKNGTDEKFQITPQYDFEVLRNLEPWTTYCVQ | |
| | VRGFLPDRNKAGEWSEPVCEQTTHDETVPSWMVAVILM | |
| | ASVFMVCLALLGCFALLWCVYKKTKYAFSPRNSLPQHL | |
| | KEFLGHPHHNTLLFFSFPLSDENDVFDKLSVIAEDSES | |
| | GKQNPGDSCSLGTPPGQGPQS | |
| hIL-10Rβ | MVPPPENVRMNSVNFKNILQWESPAFAKGNLTFTAQYL | 357 |
| Mature-No Signal | SYRIFQDKCMNTTLTECDFSSLSKYGDHTLRVRAEFAD | |
| Peptide | EHSDWVNITFCPVDDTIIGPPGMQVEVLADSLHMRFLA | |
| | PKIENEYETWTMKNVYNSWTYNVQYWKNGTDEKFQITP | |
| | QYDFEVLRNLEPWTTYCVQVRGELPDRNKAGEWSEPVC | |
| | EQTTHDETVPSWMVAVILMASVFMVCLALLGCFALLWC | |
| | VYKKTKYAFSPRNSLPQHLKEFLGHPHHNTLLFFSFPL | |
| | SDENDVFDKLSVIAEDSESGKQNPGDSCSLGTPPGQGP | |
| | QS | |

In some embodiments, the hIL-10R binding agent (e.g., the hIL-10R binding protein) (or functional fragment and/or functional variant thereof) specifically binds hIL-10Rα. In some embodiments, the hIL-10R binding agent (e.g., the hIL-10R binding protein) (or functional fragment and/or functional variant thereof) specifically binds hIL-10Rβ. In some embodiments, the hIL-10R binding agent (e.g., the hIL-10R binding protein) (or functional fragment and/or functional variant thereof) specifically binds both hIL-10Rα and hIL-10Rβ. In some embodiments, the hIL-10R binding agent (e.g., the hIL-10R binding protein) (or functional fragment and/or functional variant thereof) specifically binds both hIL-10Rα and hIL-10Rβ and binds hIL-10Rβ with higher affinity than hIL-10Rα. In some embodiments, the hIL-10R binding agent (e. g., the hIL-10R binding protein) (or functional fragment and/or functional variant thereof) specifically binds both hIL-1 ORα and hTL-10Rβ and binds hIL-10Rα with higher affinity than hIL-10Rβ.

In some embodiments, the hIL-10R binding agent (e.g., the hIL-10R binding protein) (or functional fragment and/or functional variant thereof) specifically binds both hIL-10Rα and hIL-10β and binds hIL-10Rβ with higher affinity than hIL-10Rα. In some embodiments, the hIL-10R binding agent (e.g., the hIL-10R binding protein) (or functional fragment and/or functional variant thereof) specifically binds both hIL-10Rα and hIL-10Rβ and binds hIL-10Rβ with at least about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, or 1000-fold higher affinity than hIL-10Rα. In some embodiments, the hIL-10R binding agent (e.g., the hIL-10R binding protein) (or functional fragment and/or functional variant thereof) specifically binds both hIL-10Rα and hIL-10Rβ and binds hIL-10Rβ with about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, or 1000-fold higher affinity than hIL-10Rα. In some embodiments, the hIL-10R binding agent (e.g., the hIL-10R binding protein) (or functional fragment and/or functional variant thereof) specifically binds both hIL-10Rα and hIL-10Rβ and binds hIL-10Rβ with from about 1-1000-fold, 2-1000-fold, 3-1000-fold, 4-1000-fold, 5-1000-fold, 6-1000-fold, 7-1000-fold, 8-1000-fold, 9-1000-fold, 10-1000-fold, 20-1000-fold, 30-1000-fold, 40-1000-fold, 50-1000-fold, 100-1000-fold, or 500-1000-fold higher affinity than hIL-10Rα.

In some embodiments, the hIL-10R binding agent (e.g., the hIL-10R binding protein) (or functional fragment and/or functional variant thereof) is a hIL-10R agonist. In some embodiments, the hIL-10R binding agent (e.g., the hIL-10R binding protein) (or functional fragment and/or functional variant thereof) is a hIL-10Rα agonist. In some embodiments, the hIL-10R binding agent (e.g., the hIL-10R binding protein) (or functional fragment and/or functional variant thereof) is a hIL-10Rβ agonist. In some embodiments, the hIL-10R binding agent (e.g., the hIL-10R binding protein) (or functional fragment and/or functional variant thereof) is a hIL-10Rα agonist and a hIL-10Rβ agonist. In some embodiments, the hIL-10R binding agent (e.g., the hIL-10R binding protein) (or functional fragment and/or functional variant thereof) is a hIL-10Rα agonist and a hIL-10Rβ agonist and has a greater agonistic effect on hIL-10Rβ than hIL-10Rα.

In some embodiments, the hIL-10R binding agent (e.g., the hIL-10R binding protein) (or functional fragment and/or functional variant thereof) comprises hIL-10 (or a functional fragment and/or a functional variant thereof). In some embodiments, the hIL-10R binding agent (e.g., the hIL-10R binding protein) (or functional fragment and/or functional variant thereof) comprises a viral IL-10 (vIL-10) (or a functional fragment and/or a functional variant thereof). In some embodiments, the vIL-10 is or is derived from a parapoxvirus IL-10, cytomegalovirus IL-10, gammaherpesvirus IL-10, orf virus IL-10, pseudocowpox virus IL-10, betaherpesvirus IL-10, or an Epstein-Barr virus IL-10. In some embodiments, the viral IL-10 is or is derived from a human herpes virus IL-10 (e.g., a cytomegalovirus or an Epstein-Barr virus).

The amino acid sequence of exemplary hIL-10R binding proteins (hIL-10R BPs) is set forth in Table 2. The amino acid sequence of the immature form of the exemplary hIL-10R binding proteins (i.e., containing the native signal peptide) is set forth in SEQ ID NOS: 1-178. The amino acid sequence of the mature form of the exemplary hIL-10R binding proteins (i.e., lacking the native signal peptide) is set forth in SEQ ID NOS: 179-353.

The signal peptides have been computationally predicted for hIL-10R BP-4-11 and 15-178 using standard methods (see, e.g., Teufel, F., Almagro Armenteros, J. J., Johansen, A. R. et al. SignalP 6.0 predicts all five types of signal peptides using protein language models. Nat Biotechnol (2022). https://doi.org/10.1038/s41587-021-01156-3, the entire contents of which is incorporated by reference herein for all purposes). A person of ordinary skill in the art would know how to experimentally identify and/or validate a computationally predicted signal peptide using standard methods known in the art, e.g., expression of the hIL-10R binding protein from a host cell and sequencing of the intracellular form and the extracellular form of the expressed protein (see, e.g., Zhang Z, Henzel W J. Signal peptide prediction based on analysis of experimentally verified cleavage sites. Protein Sci. 2004; 13(10):2819-2824. doi:10.1110/ps.04682504, the entire contents of which is incorporated herein by reference for all purposes).

TABLE 2
The Amino Acid Sequence of hIL-10R Binding Proteins.

| | | SEQ |
|---|---|---|
| Description | Amino Acid Sequence | ID NO |
| hIL-10R BP-1 | MHSSALLCCLVLLTGVRASPGQGTQSENSCTHFPGNLPNMLRDLRDAFS | 1 |
| with signal | RVKTFFQMKDQLDNLLLKESLLEDFKGYLGCQALSEMIQFYLEEVMPQA | |
| peptide | ENQDPDIKAHVNSLGENLKTLRLRLRRCHRFLPCENKSKAVEQVKNAEN | |
| | KLQEKGIYKAMSEFDIFINYIEAYMTMKIRN | |
| hIL-10R BP-2 | MHSSALLCCLVLLTGVRASPGQGTQSENSCTHFPGYLPNMLRDLRDAFS | 2 |
| with signal | RVKTFFQMKDQLDNLLLKESLLEDFKGYLGCQALSEMIQFYLEEVMPQA | |
| peptide | ENQDPDIKAHVQSLGENLKDLRLWLRRCHRFLPCENKSKAVEQVKNAFN | |
| | KLQEKGIYKAMSEFDIFINYIEAYMTMKIRN | |
| hIL-10R BP-3 | MHSSALLCCLVLLTGVRASPGQGTQSENSCTHEPGNLPNMLRALRDAFS | 3 |
| with signal | RVKTFFQMKDQLDNLLLKESLLEDFKGYLGCQALSEMIQFYLEEVMPQA | |
| peptide | ENQDPDIKAHVNSLGENLKTLRLRLRRCHRFLPCENKSKAVEQVKNAEN | |
| | KLQEKGIYKAMSEFDIFINYIEAYMTMKIRN | |
| hIL-10R BP-4 | MGKRAFVVSVAMALLGIYVITNTVNARHCMFGDSLRNSPDMKNMLQDLR | 4 |
| with signal | GGYSGSGIKRTFQGKDTLDSMLLTQSLLDDEKGYLGCQALSEMIQFYLE | |
| peptide | EVMPQAENHGPTDSVKQLGEKLHTLNQKFGECPRWFPCYYNTTPAVENV | |
| | KSVFSKLQERGVYKAMSEFDIFINYIETYTTMK | |
| hIL-10R BP-5 | MARRLTVASCGSVSLLAAFAAVLLIGCQLESGEALPLGSRSADSRSVDG | 5 |
| with signal | QRVPAPQNNYPGLLRDLRLGYEGFKQKVTDSHPDETLLGSSRLAGDLKG | |
| peptide | PLRCQALSEMIQFLLQVVLPDAENSRQDLRSQFSTLGDRITGLRQQLRR | |
| | DPTVFPCESRSDGVSDLRSAYTRLGSTGAEKVLSEFDIFINYIEAYVTS | |
| | V | |
| hIL-10R BP-6 | MSNNKILVCAVIILTYTLYTDAYCVEYAESDEDRQQCSSSSNFPASLPH | 6 |
| with signal | MLRELRAAFGKVKTFFQMKDQLNSMLLTQSLLDDEKGYLGCQALSEMIQ | |
| peptide | FYLEEVMPQAENHGPDIKEHVNSLGEKLKTLRLRLRRCHRELPCENKSK | |
| | AVEQVKRVFNMLQERGVYKAMSEFDIFINYIESYMTTKM | |
| hIL-10R BP-7 | MANVVYVVLVISIMMANIHVSKTYCTSCSHHQCTEDENQKQDCEDANHS | 7 |
| with signal | LPHMLRELRAAFGKVKTFFQMKDQLHSLLLTQSLLDDFKGYLGCQALSE | |
| peptide | MIQFYLEEVMPQAENHGPEEHDNSLSEHGPDVKEHVNSLGEKLKTLRLR | |
| | LRRCHRFLPCENKSKAVEKVKRVFSELQERGVYKAMSEFDIFINYIETY | |
| | MTT | |
| hIL-10R BP-8 | MQGLQLLRGLLCCGVFAAASSRSPKNKPSIDCNPQTGDFVNMLKSMRQD | 8 |
| with signal | YSRIRDTLHDRDKLHSSLLTGALLDEMMGYSGCRTTLLLMEHYLDTWYP | |
| peptide | AAYRHHLYDNQTLVVVDRMGSTLVALLKAMVQCPMLACGAPSPAMDKML | |
| | QQEAKMKKYTGVYKGISETDLLLGYLELYMMKFKR | |
| hIL-10R BP-9 | MRRRRSFGIVVSGAIRTLLMVAVVAVSVRGHEHKVPPACDPVHGNLAGI | 9 |
| with signal | FKELRAIYASIREALQKKDTVYYTSLENDRVLQEMLSPMGCRVTNELME | |
| peptide | HYLDGVLPRAAHEDYDNSTLNGLHAFTSSMQALYQHMLKCPALACTGKT | |
| | PAWMYFLEVEHKLNPWRGTAKAAAEADLLLNYLETFLLQF | |
| hIL-10R BP-10 | MGSRRLSRCSFATAVCLVAIVAAVAAKGRDSKPSPACDPMHGALAGIFK | 10 |
| with signal | ELRTTYRSVREALQTKDTVYYVSLFHEQLLQEMLSPVGCRVTNELMQHY | |
| peptide | LDGVLPRAFHCGYDNATLNALHALSSSLSTLYQHMLKCPALACTGQTPA | |
| | WTQFLDTEHKLDPWKGTVKATAEMDLLLNYLETFLLQS | |
| hIL-10R BP-11 | MLSVMVSSSLVLIVFFLGASEEAKPATTTIKNTKPQCRPEDYATRLQDL | 11 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-12 | MALAHQLPVWIFSIWILYFTLPLSEERVLPLRGNCKLLLQDTVIPNLLY | 12 |
| with signal | SMRSIFQDIKPYFQGKDSLNNLLLSGQLLEDLQSPIGCDALSEMIQFYL | |
| peptide | EEVMPQAEIHHPKHKNSVMQLGETLHTLISQLQECTALFPCKHKSLGAQ | |
| | KIKEEVSKLGQYGIIKAVAEFDIFINYMESYFGVK | |
| hIL-10R BP-13 | MRRRRRSFGIIVAGAIGTLLMMAVVVLSAHDHEHKEVPPACDPVHGNLA | 13 |
| with signal | GIFKELRATYASIREGLQKKDTVYYTSLENDRVLHEMLSPMGCRVTNEL | |
| peptide | MEHYLDGVLPRASHLDYDNSTLNGLHVFASSMQALYQHMLKCPALACTG | |
| | KTPAWMYFLEVEHKLNPWRGTAKAAAEADLLLNYLETFLLQF | |
| hIL-10R BP-14 | MRRRRSFGIVVAGAIGTLLMMAVVVESAHEHKEVPPACDPVHGNLAGIF | 14 |
| with signal | KELRATYASIREGLQKKDTVYYTSLFNDRVLQEMLSPMGCRVTNELMEH | |
| peptide | YLDGVLPRALHLDYDNSTLNGLHAFASSMQALYQHMLKCPALACTGKTP | |
| | AWMYFLEVEHKLNPWRGTAKAAAEADLLLNYLETFLLQF | |
| hIL-10R BP-15 | MSKNKILVCVVIILTYTLYTDAYCVEYEESEEDRQQCSSSNFPASLPHM | 15 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKP | |
| hIL-10R BP-16 | MSNNKILVCVVIILTYTLYTDAYCVEYEESEEDRQQCSSSNFPASLPHM | 16 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKT | |
| hIL-10R BP-17 | MPNNKILVCAVIILTYTLYTDAYCVEYEESEEDRQQCSSSNFPASLPHM | 17 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKS | |
| hIL-10R BP-18 | MSNKKILVCVVIILTYTLYTDAYCVEYKESEEDRQQCSSSSFPASLPHM | 18 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTK | |
| hIL-10R BP-19 | MSNNKILVCVAIILTYTLYTDAYCVEYAESDEDKQQCSGSNFPASLPHM | 19 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKM | |
| hIL-10R BP-20 | MSKNKVLVCFVIILTYTLYTDAYCVEYEESEEDKQQCGSNGGPASLPHM | 20 |
| with signal | LRELRAAFGKVKTFFQMKDQLNSMLLTQSLLDDEKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKM | |
| hIL-10R BP-21 | MSNNKILVCAVIILTYTLYTDAYCVEYAESDEDRQQCSGSNFPASLPHM | 21 |
| with signal | LRELRAAFGKVKTFFQMKDQLNSMLLTQSLLDDEKGYLGCQALSEMIQF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKM | |
| hIL-10R BP-22 | MSNNKILLCVAIILTYTLYTDAYCVEYEESEEDKQQCSSSSNFPASLPH | 22 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-23 | MSNNKILVCAVIILTYTLYTDAYCIQYEESEEDKQQCSSSNFPASLPHM | 23 |
| with signal | LRELRAAFGKVKTFFQMKDQLNSMLLTQSLLDDEKGYLGCQALSEMIRF | |
| peptide | YLEEVMPQAENHGPDIKEHVNSLGEKLKTLRLRLRRCHRFLPCENKSKA | |
| | VEQVKRVFNMLQERGVYKAMSEFDIFINYIESYMTTKM | |
| hIL-10R BP-24 | MSKNKFLVCVVIILTYTLYTDAYCVEYEESEEDRQQCSSSNFPASLPHM | 24 |
| with signal | LRELRAAFGKVKTFFQMKDQLNSMLLTQSLLDDEKGYLGCQALSEMIQF | |
| peptide | YLEEVMPQAENHGPDIKEHVNSLREKLKTLRLRLRRCHRFLPCENKSKA | |
| | VEQVKRVFNMLQERGVYKAMSEFDIFINYIESYMTTKT | |
| hIL-10R BP-25 | MSKNKILVCFVIILTYTLYTDAYCVEYEESEEDKQQCGSSSNFPASLPH | 25 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-26 | MSNNKILVCVAIILTYTLYTDAYCVEYAESDEDKQQCSGSNFPASLPHM | 26 |
| with signal | LRELRAAFGKVKTFFQMKDQLNSMLLTQSLLDDEKGYLGCQALSEMIQF | |
| peptide | YLEEVMPRAENHGPDIKEHVNSLGEKLKTLRLRLRRCHRFLPCENKSKA | |
| | VEQVKRVFNMLQERGVYKAMSEFDIFINYIESYMTTKM | |
| hIL-10R BP-27 | MSNNKILVCVVIILTYTLYTDAYCVEYEESEEDRQQCSGSSNFPASLPH | 27 |
| with signal | MLRELRAAFGKVKTFFQMKDQLNSMLLTQSLLDDEKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-28 | MSNNKILVCAVIILTYTLYTDAYCVEYEESDEDRQQCSSSSNFPASLPH | 28 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-29 | MSKNKILVCVAIILTYTLYTDAYCVEYEESDEDKQQCSSSTGAPASLPH | 29 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-30 | MSKNKILVCVAIILTYTLYTDAYCVEYEETKEDEQQCSSSSNFPASLPH | 30 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRELPCENKSK | |
| | AVEQVKRVFNMLQERGVYKAMSEFDIFINYIESYMTTKM | |
| hIL-10R BP-31 | MSKNKILVCVVIILTYTLYTDAYCVEYEESEEDRQQCSSSNFPASLPHM | 31 |
| with signal | LRELRAAFGKVKTFFQMKDQLNSMLLTQSLLDDFKGYLGCQALSEMIQF | |
| peptide | YLEEVMPQAENHGPDIKEHVNSLGEKLKTLRLRLRRCHRFLPCENKSKA | |
| | VEQVKRVFNMLQERGDYKAMSEFDIFINYIESYMTTKS | |
| hIL-10R BP-32 | MSNNKILVCVVIILTYTLYTDAYCVEYEESEEDRQQCSSSSNFPASLPH | 32 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRELPCEDKSK | |
| | AVEQVKRVFNMLQERGVYKAMSEFDILINYIESYMTTKM | |
| hIL-10R BP-33 | MSNNKILVCAVIILTYTLYTDAYCVEYEESDEDRQQCSSSSNFPASLPH | 33 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMATKM | |
| hIL-10R BP-34 | MERALLLCCLALLAGVWADNRYDGQDGNDCPTLPTSLPHMLHELRAAFS | 34 |
| with signal | RVKTFFQMKDQLDNMLLDGSLLEDFKGYLGCQALSEMIQFYLEEVMPQA | |
| peptide | ENHSTDQEKDKVNSLGEKLKTLRVRLRRCHRFLPCENKSKAVEQVKSAF | |
| | SKLQEKGVYKAMSEFDIFINYIEAYMTTKMKN | |
| hIL-10R BP-35 | MSKNKILVCVAIILTYTLYTDAYCVEYLESREDEQQCSSSSNFPASLPH | 35 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRELPCENKSK | |
| | AVEQVKRVFNMLQERGVYKAMSEFDIFINYIESYMTTKM | |
| hIL-10R BP-36 | MSKNKILVCVAIILTYTLYTDAYCVEYEESKEDEQQCSGSNGASASLPH | 36 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-37 | MSKNKILVCVAIILTYTLYTDAYCVEYLESGEDEQQCGSSSNFPASLPH | 37 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-38 | MSKNKILVCVAIILTYTLYTDAYCVEYLESREDEQQCSGSNGASASLPH | 38 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRFLPCENKSK | |
| | AVEQVKRVFNMLQERGVYKAMSEFDIFINYIESYMTTKM | |
| hIL-10R BP-39 | MPGAALLYCLFFVTGVWAESENNCTHFPTSLPHMLHELRAAFSRVKTFF | 39 |
| with signal | QMKDQLDNMLLNGSLLEDFKGYLGCQALSEMIQFYLEEVMPQAENHSGG | |
| peptide | GGPDIKEHVNSLGEKLKTLRVRLRRCHRFLPCENKSKAVEQVKSAFSKL | |
| | QEKGVYKAMSEFDIFINYIEAYMTTKMKNKK | |
| hIL-10R BP-40 | MSNNKILVCVAIILTYTLYTDAYCVEYLESDEDKQHCSSSNGASASSPH | 40 |
| with signal | MLRELRAAFGKVKTFFQMKDQLNSMLLTQSLLDDFKGYLGCQALSEMIQ | |
| peptide | FYLEEVMPQAENHGPDIKEHVNSLGEKLKTLRLRLRRCHRELPCENKSK | |
| | AVEQVKRVFNMLQERGVYKAMSEFDIFINYIESYMTTKM | |
| hIL-10R BP-41 | MSNNKILVCVAIILTYTLYTNAYCVEYLESEEDKQQCGSNGASSSSPHM | 41 |
| with signal | LRELRAAFGKVKTFFQMKDQLNSMLLTQSLLDDFKGYLGCQALSEMIQF | |
| peptide | YLEEVMPQAENHGPDVKEHVNSLAEKLKTLRLRLRRCHRFLPCENKSKA | |
| | VEQVKRVFNMLQERGVYKAMSEFDIFINYIESYMTTKS | |
| hIL-10R BP-42 | MSNNKILVCVAIILTYTLYTNAYCVEYLESEEDKQQCGSNGASSSSPHM | 42 |
| with signal | LRELRAAFGKVKTFFQMKDQLNSMLLTQSLLDDFKGYLGCQALSEMIQF | |
| peptide | YLEEVMPQAENHGPDVKEHVNSLGEKLKTLRLRLRRCHRFLPCENKSKA | |
| | VAQVKRVFNMLQERGVYKAMSEFDIFINYIESYMTTKS | |
| hIL-10R BP-43 | ILVCFVIILTYTLYTDAYCVEYEESEEDRQQCSSSNFPASLPHMLRELR | 43 |
| with signal | AAFGKVKTFFQMKDQLNSMLLTQSLLDDFKGYLGCQALSEMIQFYLEEV | |
| peptide | MPQAENHGPDIKEHVNSLGEKLKTLRLRLRRCHRFLPCENKSKAVEQVK | |
| | RVFNMLQERGVYKAMSEFDIFINYIES | |
| hIL-10R BP-44 | ILVCFVIILTYTLYTDAYCVEYEESEEDRQQCSSSNFPASLPHMLRELR | 44 |
| with signal | AAFGKVKTFFQMKDQLNSMLLTQSLLDDFKGYLGCQAFSEMIQFYLEEV | |
| peptide | MPQAENHGPDIKEHVNSLGEKLKTLRLRLRRCHRELPCENKSKAVEQVK | |
| | RVFNMLQERGVYKAMSEFDIFINYIES | |
| hIL-10R BP-45 | LVCVAIILTYTLYTDAYCVEYLESREDEQQCSSSSNFPASLPHMLRELR | 45 |
| with signal | AAFGKVKTFFQMKDQLNSMLLTQSLLDDFKGYLGCQALSEMIQFYLEEV | |
| peptide | MPQAENHGPDIKEHVNSLGEKLKTLRLRLRRCHRFLPCENKSKAVEQVK | |
| | RVFNMLQERGVYKAMSEFDIFINYIES | |
| hIL-10R BP-46 | MGLRSGLTLQCLVILQCLVMLYLAPACKGVSNCGNLPHMLRDLRDAFSR | 46 |
| with signal | VKTFFQMKDQLDNILLKESLLEDFKGYLGCQALSEMIQFYLEEVMPQAE | |
| peptide | NQDPHAKEHVNSLGENLKTLRLRLRRCHRFLPCENKSKAVEQVKNAFSK | |
| | LQEKGVYKAMSEFDIFINYIEAYMTMKIRR | |
| hIL-10R BP-47 | LVCVAIILTYTLYTDAYCVEYLESREDEQQCGSSSNFPASLPHMLRELR | 47 |
| with signal | AAFGKVKTFFQMKDQLNSMLLTQSLLDDFKGYLGCQALSEMIQFYLEEV | |
| peptide | MPQAENHGPDIKEHVNSLGEKLKTLRLRLRRCHRFLPCENKSKAVEQVK | |
| | RVFNMLQERGVYKAMSEFDIFINYIES | |
| hIL-10R BP-48 | ILVCFVIILTYTLYTDAYCVEYEESEEDRQQCSSSNFPASLPHMLRELR | 48 |
| with signal | AAFGKVKTFFQMKDQLNSMLLTQSLLDDFKGYLGCQALSEMIQFYLEEV | |
| peptide | MPQAENHGPDIKEHVNSPGEKLKTLRLRLRRCHRFLPCENKSKAVEQVK | |
| | RVFNMLQERGVYKAMSEFDIFINYIES | |
| hIL-10R BP-49 | MANVIYVVLALNILLSQIHVSNPYCTSCSYRDCTEDEDQKQQCEGGLRS | 49 |
| with signal | LPHMLRELRAAFGKVKTFFQMKDQLHSLLLTQSLLDDFKGYLGCQALSE | |
| peptide | MIQFYLEEVMPQAENHGPDIKEHVNSLGEKLKTLRLRLRRCHRFLPCEN | |
| | KSKAVEKVKRVFSELQERGVYKAMSEFDIFINYIETYMT | |
| hIL-10R BP-50 | ILVCFVIILTYTLYTDAYCVEYEESEEDRQQCSSSNFPASLPHMPRELR | 50 |
| with signal | AAFGKVKTFFQMKDQLNSMLLTQSLLDDFKGYLGCQALSEMIQFYLEEV | |
| peptide | MPQAENHGPDIKEHVNSLGEKLKTLRLRLRRCHRFLPCENKSKAVEQVK | |
| | RVFNMLQERGVYKAMSEFDIFINYIES | |
| hIL-10R BP-51 | MANVIYVVLALNILLSQFHVSNPYCTSCSHRDCTEDDEQKQQCEGGSGG | 51 |
| with signal | LGSLPHMLRELRAAFGKVKTFFQMKDQLHSLLLTQSLLDDFKGYLGCQA | |
| peptide | LSEMIQFYLEEVMPQAENHGPDIKEHVNSLGEKLKTLRLRLRRCHRFLP | |
| | CENKSKAVEKVKRVESELQERGVYKAMSEFDIFINYIETYMT | |
| hIL-10R BP-52 | MERRLVVTLQCLVLLYLAPECGGTDQCDNFPQMLRDLRDAFSRVKTFFQ | 52 |
| with signal | TKDEVDNLLLKESLLEDFKGYLGCQALSEMIQFYLEEVMPQAENQDPGA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-53 | MERRLVVTLQCLVLLYLAPECGSTDQCDNFPQMLRDLRDAFSRVKTFFQ | 53 |
| with signal | TKDEVDNLLLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-54 | MERRLVVTLQCLVLLYLAPECGGTDQCDNFPQMLRDLRDAFSRVKTFFQ | 54 |
| with signal | TKDEVDNLLLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTMKAR | |
| hIL-10R BP-55 | MGPRAGLALQCLLLLYLAPACKGVSNCGNLPHMLRDLRDAFSRVKTFFQ | 55 |
| with signal | MKDQLDNILLKESLLEDFKGYLGCQALSEMIQFYLEEVMPQAENQDPNA | |
| peptide | KEHVNSLGENLKTLRLRLRRCHRFLPCENKSKAVEQVKNAFSKLQEKGV | |
| | YKAMSEFDIFINYIEAYMTMKTRR | |
| hIL-10R BP-56 | MERRLVVTLQCLVLLYLAPECGGTDQCDNFPQMLRDLRDAFSRVKTFFQ | 56 |
| with signal | TKDEVDNLLLKESLLEDFKGYLGCQALSEMIQFYLEEVMPQAENQDPEA | |
| peptide | KDHVNSLGENLKTIRLRLRRCHRFLPCENKSKAVEQIKNAENKLQEKGI | |
| | YKAMSEFDIFINYIEAYMTIKAR | |
| hIL-10R BP-57 | MERRLVVTLQCLVLLYLAPECGGTDQCDNFPQMLRDLRDAFSRVKTFFQ | 57 |
| with signal | TKDEVDSLLLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-58 | MERRLMVTLQCLVLLYLAPECGSTDQCDNFPQMLRDLRDAFSRVKTFFQ | 58 |
| with signal | TKDEVDNILLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-59 | MERRLVVTLQCLVLLYLAPECGGTDQCDNFPQMLRDLRDAFSRVKTFFQ | 59 |
| with signal | TKDEVDNLFLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-60 | MERRLVVTLQCLVLLYLAPECGGTDQCDNFPQMLRDLRDAFSRVKTFFQ | 60 |
| with signal | TKDEVDNLLLKESLLEDFKGYLGCQALSEMIQFYLEKVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-61 | MERRLMVTLQCLVLLYLAPECGSTDQCDNFPQMLRDLRDAFSRVKTFFQ | 61 |
| with signal | TKDAVDNLLLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFDIFINYIEAYMTIKAR | |
| hIL-10R BP-62 | MELRSGLTLQCLVMLQCLVMLYLAPACKGASNCGNLPHMLRDLRDAFSR | 62 |
| with signal | VKTFFQMKDQLDNILLKESLLEDFRGYLGCQALSEMIQFYLEEVMPQAE | |
| peptide | NQDPHSKEHVNSLGENLKTLRLRLRRCHRFLPCENKGKAVEQVKNAFSK | |
| | LQEKGVYKAMSEFDIFINYIEAYMTMKLRR | |
| hIL-10R BP-63 | MERRLVVTLQCLVLLYLAPECGGTDQCDNFPQMLRDLRDAFSRVKTFFQ | 63 |
| with signal | TKDEVDNLLLKESLLEDFKGYLGCQALSEMIQFYLEEVMPQAENQDPEA | |
| peptide | KDHVNSLGENLKTLRLRLRRCHRFLPCENKSKAVEQIKNAFNKLQEKGI | |
| | YKAMSEFHIFINYIEAYMTIKAR | |
| hIL-10R BP-64 | MERRLVVTLQCLVLLYLAPECGEMLRDLRDAFSRVKTFFQTKDEVDNLL | 64 |
| with signal | LKESLLEDFKGYLGCQALSEMIQFYLEEVMPQAENQDPEAKDHVNSLGE | |
| peptide | NLKTLRLRLRRCHRFLPCENKSKAVEQIKNAFNKLQEKGIYKAMSEFDI | |
| | FINYIEAYMTIKAR | |
| hIL-10R BP-65 | MELSLGLTLHFLVFLCLAPACGRAETCGNIPHMLRDLRDAFSRVKTFFQ | 65 |
| with signal | MKDQLDNILLKESLLEDFKGYLGCQALSEMIQFYLEEVMPQAEAMSLKS | |
| peptide | QEHVNFLGENLNTLRLRLRRCHRFLPCENKSKAVEQVKNAFSKLQEKGV | |
| | YKAMSEFDIFINYIEAYMTMKLRR | |
| hIL-10R BP-66 | MAYGKKIVAASLLVIPAYVVFTNATANNRAQKCFCFDGSNAGNSEETNT | 66 |
| with signal | AAFQKKCDSEIPESLPYMLRDLRNSSVQTRRYFQEKDEENSPLLTQKLL | |
| peptide | EDFKGYLGCQALSEMIQFYLEEVMPQAEDSNPSAKDSVTSLGEKLKTLR | |
| | LRLRRCHRFLPCENKSKAVENLKSKFGDLGNQGVHKAMSEFDIFINYIE | |
| | TYMTTKMK | |
| hIL-10R BP-67 | MLSVMVSSSLVLIVFFLGASEEAKPATTTTIKNTKPQCRPEDYATRLQD | 67 |
| with signal | LRVTFHRVKPTLQREDDYSVWLDGTVVKGCWGCSVMDWLLRRYLETVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-68 | MLSVMVSSSLVLIVFFLGASEEAKPATTTTIKNTKPQCRPEDYATRLQD | 68 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-69 | MLSVMVSSSLVLIVFFLGASEEAKPATTTTKNTKPQCRPEDYATRLQDL | 69 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-70 | MLSVMVSSSLVLIVFFLGASEEAKPATTTTIKNTKPQCRPEDYATRLQD | 70 |
| with signal | LRVTFHRVKPTLQREDDYSVWLDGTMVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-71 | MLSVMVSSSLVLIVFFLGASEEAKPATTTTIKNTKPQCRPEDYATRLQD | 71 |
| with signal | LRVTFHRVKSTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-72 | MLSVMVSSSLVLIVFFLGASEEAKPATTTTIKNTKPQCRPEDYATRLQD | 72 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDMLFSRLEEYLHSRK | |
| hIL-10R BP-73 | MLSVMVSSSLVLIVFFLGASEEAKPATTTTIKNTKPQCRPEDYATRLQD | 73 |
| with signal | LRVTFHRVKPTLQREDDYSVWLDGTVVKGCWGCSVMDWLLRRYLEIVLP | |
| peptide | AGDHVYPGLKTELHSMRSTLESIYKDMRQCPLLGCGDKSVISRLSQEAE | |
| | RKSDNGTRKGLSELDTLESRLEEYLHSRK | |
| hIL-10R BP-74 | MLSVMVSSSLVLIVFFLGASEEAKPAATTTIKNTKPQCRPEDYATRLQD | 74 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLESRLEEYLHSRK | |
| hIL-10R BP-75 | MLSVMVSSSLVLIVFFLGASEEAKPATTTTINNTKPQCRPEDYATRLQD | 75 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-76 | MLSVMVSSSLVLIVFFLGASEEAKPATTTTIKNTKPQCRPEDYATRLQD | 76 |
| with signal | LRVTFHRVKPTLQREDDYSVWLDGTVVKGCWGCSVMDWLLRRYLEIVFP | |
| peptide | AGDHVYPGLKTELHSMHSTLESIYKDMRQCPLLGCGDKSVISRLSQEAE | |
| | RKSDNGTRKGLSELDTLFSRLEEYLHSRK | |
| hIL-10R BP-77 | MLSVMVSSSLVLIIFFLGASEEAKPATTTTIKNTKPQCRPEDYATRLQD | 77 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGMRKGLSELDTLFSRLEEYLHSRK | |
| hIL-10R BP-78 | MLSVMVSSSLVLIVFFLGASEEAKPATTTIKNTKPQCRPEDYATRLQDL | 78 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-79 | MLSVMVSSSLVLIVFFLGASEEAKPATTTTIKNTKPQCRPEDYATRLQD | 79 |
| with signal | LRVTFDRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLESRLEEYLHSRK | |
| hIL-10R BP-80 | MLSVMVSSSLVLIVFFLGASEEAKPAATTTIKNTKPQCRPEDYATRLQD | 80 |
| with signal | LRVTFHRVKPTLQHEDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-81 | MLSVMVSSSLVLIVFFLGASEEAKPATTTTIKNTKPQCRPEDYATRLQD | 81 |
| with signal | LRVTFHRVKPTLQREDDYSVWLDGMVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-82 | MLSVMVSSSLVLIVFFLGASEEAKPATTTTIKNTKPQCRPEDYATRLQD | 82 |
| with signal | LRVTFHRVKPTLQREDDYSVWLDGTVVKGCWGCSVVDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-83 | MLSVMVSSSLVLIVFFLGASEEAKPATTTTIKNTKPQCRPEDYATRLQD | 83 |
| with signal | LRITFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-84 | MLSVMVSSSLVLIVFFLGASEEAKPATTTIKNTKPQCRPEDYATRLQDL | 84 |
| with signal | RVTFHRIKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-85 | MLSVMVSSSLVLIVFFLGASEEAKPATTTTIKNTKPQCRPEDYATRLQD | 85 |
| with signal | LRVTFHRVKPTLQCEDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-86 | MLSVMVFSSLVLIVFFLGASEEAKPATTTTTIKNTKPQCRPEDYATRLQ | 86 |
| with signal | DLRVTFHRVKPTLQREDDYSVWLDGTVVKGCWGCSVMDWLLRRYLEIVF | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-87 | MLSVMVSSSLVLIVFFLGASEEAKPATTTTIKNTKPQCRPEDYATRLQD | 87 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDALFSRLEEYLHSRK | |
| hIL-10R BP-88 | MLSVMVSSSLVLIVFFLGASEEAKPATTTTIKNTKPQCRPEDYATRLQD | 88 |
| with signal | LRVTFHRVKPTLQREDDYSVWLDGTVVKGCWGCSVMDWLLRRYLEILEP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-89 | MLSVMVSSSLVLIVFFLGASEEAKPATTTTTIKNTKPQCRPEDYATRLQ | 89 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGMRKGLSELDTLFSRLEEYLHSRK | |
| hIL-10R BP-90 | MLSVMVSSSLVLIVFFLGASEEAKPATTTIKNTKPQCRPEDYATRLQDL | 90 |
| with signal | RVTFHRVKPTLQREDDYSVWLDGTVVKGCWGCSVMDWLLRRYLEIVFPA | |
| peptide | GDHVYPRLKTELHSMRSTLESIYKDMQQCPLLGCGDKSVISRLSQEAER | |
| | KSDNGTRKGLSELDTLFSRLEEYLHSRK | |
| hIL-10R BP-91 | MLSVMVSSSLVLIVFFLGASEEAKPAATTTTIKNTKPQCRPEDYATRLQ | 91 |
| with signal | DLRVTFHRVKPTLQREDDYSVWLDGTVVKGCWGCSVMDWLLRRYLEIVF | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-92 | MLSVMVSSSLVLIVFFLGASEEAKPAATTTTTIKNTKPQCRPEDYATRL | 92 |
| with signal | QDLRVTFHRVKPTLQREDDYSVWLDGTVVKGCWGCSVMDWLLRRYLEIV | |
| peptide | FPAGDHVYPGLKTELHSMRSTLESIYKDMRQCPLLGCGDKSVISRLSQE | |
| | AEKKSDNGTRKGLSELDTLFSRLEEYLHSRK | |
| hIL-10R BP-93 | MLSVMVSSSLVLIVFFLGASEEAKPAATTTTIKNTKPQCRPEDYASRLQ | 93 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-94 | MLSVMVSSSLVLIVFFLGASEEAKPATTTTTIKNTKPQCRPEDYATRLQ | 94 |
| with signal | DLRVTFHRVKPTLQREDDYSVWLDGTVVKGCWGCSVMDWLLRRYLEIME | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-95 | MLSVMVSSSLVLIVFFLGASEEAKSATTTIKNTKPRCRPEDYATRLQDL | 95 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-96 | MLSVMVSSSLVLIIFFLGASEEAKPATTTTIKNTKPQCRPEDYATRLQD | 96 |
| with signal | LRVTFHRVKPTLQREDDYSVWLDGTVVKGCWGCSVMDWLLRRYLEIVFP | |
| peptide | AGDYVYPGLKTELHSMRSTLESIYKDMRQCPLLGCGDKSVISRLSQEAE | |
| | RKSDNGMRKGLSELDTLFSRLEEYLHSRK | |
| hIL-10R BP-97 | MLSVMVSSSLVLIVFFLGASEEAKPATTTIKNTKPQCRPEDYATRLQDL | 97 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDALFSRLEEYLHSRK | |
| hIL-10R BP-98 | MLSVMVSSSLVLIVFFLGASEEAKPATTTTTIKNTKPRCRPEDYATRLQ | 98 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-99 | MLSVMVSSSLVLIVFFLGASEEAKPAATTTTTIKNTKPQCRPEDYATRL | 99 |
| with signal | QDLRVTFHRVKPTLQREDDYSVWLDGTVVKGCWGCSVMDWLLRRYLEIV | |
| peptide | FPAGDHVYPGLKTELHSMRSTLESIYKDMRQCPLLGCGDKSVISRLSQE | |
| | AERKSDNGTRKGLSELDTLFSRLEEYLHSRK | |
| hIL-10R BP-100 | MLSVMVSSSLVLIIFFLGASEEAKPATTTTTTIKNTKPQCRPEDYATRL | 100 |
| with signal | QDLRVTFHRVKPTLQREDDYSVWLDGTVVKGCWGCSVMDWLLRRYLEIV | |
| peptide | FPAGDHVYPGLKTELHSMRSTLESIYKDMRQCPLLGCGDKSVISRLSQE | |
| | AERKSDNGTRKGLSELDTLFSRLEEYLHSRK | |
| hIL-10R BP-101 | MLSVMVSSSLVLIVFFLGASEEAKPAATTTIKNTKPQCRPEDYATRLQD | 101 |
| with signal | LRVTFHRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLNELDTLFSRLEEYLHSRK | |
| hIL-10R BP-102 | MLSVMVSSSLVLIVFFLGASEEAKPATTTTTIKNTKPQCRPEDYATRLQ | 102 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDALFSRLEEYLHSRK | |
| hIL-10R BP-103 | MLSVMVSSSLVLIVFFLGASEEAKPATTTTIKNTKPQCRPEDYATRLQD | 103 |
| with signal | LCVTFHRVKPTLQREDDYSVWLDGTVVKGCWGCSVMDWLLRRYLEIVFE | |
| peptide | PAGDHVYPGLKTELHSMRSTLESIYKDMRQCPLLGCGDKSVISRLSQEA | |
| | RKSDNGTRKGLSELDTLESRLEEYLHSRK | |
| hIL-10R BP-104 | MLSVMVSSSLVLIVFFLGASEEAKPATTTTTTTIKNTKPQCRPEDYATR | 104 |
| with signal | LQDLRVTFHRVKPTLQREDDYSVWLDGTVVKGCWGCSVMDWLLRRYLEI | |
| peptide | VFPAGDHVYPGLKTELHSMRSTLESIYKDMRQCPLLGCGDKSVISRLSQ | |
| | KAERKSDNGTRKGLSELDTLFSRLEEYLHSRK | |
| hIL-10R BP-105 | MLSVMVSSSLVLIVFFLGASEEAKPAATTTTIKNTKPQCRPEDYASRLQ | 105 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKSLSELDTLFSRLEEYLHSRK | |
| hIL-10R BP-106 | MLSVMVSSSLVLIVFFLGASEKAKSATTTIKNTKPQCRPEDYATRLQDL | 106 |
| with signal | RVTFHRVKPTLQREDDYSVWLDGTVVKGCWGCSVMDWLLRRYLEIVFPA | |
| peptide | GDHVYSGLKTELHSMRSTLESIYKDMRQCPLLGCGDKSVISRLSQEAER | |
| | KSDNGTRKGLSELDTLFSRLEEYLHSRK | |
| hIL-10R BP-107 | MLSVMVSSSLVLIVFFLGASEEAKPAATTTTTTIKNTKPQCRPEDYATR | 107 |
| with signal | LQDLRVTFHRVKPTLQREDDYSVWLDGTVVKGCWGCSVMDWLLRRYLEI | |
| peptide | VFPAGDHVYPGLKTELHSMRSTLESIYKDMRQCPLLGCGDKSVISRLSQ | |
| | KAERKSDNGTRKGLSELDTLESRLEEYLHSRK | |
| hIL-10R BP-108 | MLSVMVSSSLVLIVFFLGASEEAKPAATTTTTMIKNTKPQCRPEDYATR | 108 |
| with signal | LQDLRVTFHRVKPTLQREDDYSVWLDGTVVKGCWGCSVMDWLLRRYLEI | |
| peptide | VFPAGDHVYPGLKTELHSMRSTLESIYKDMRQCPLLGCGDKSVISRLSQ | |
| | EAERKSDNGTRKGLSELDTLFSRLEEYLHSRK | |
| hIL-10R BP-109 | MLSVMVSSSLVLIVFFLGASEEAKPATTTTTIKNTKPQCRPEDYATRLQ | 109 |
| with signal | DLRVTFHRVKPTLQREDDYSVWLDGTVVKGCWGCSVMDWLLRRYLEIVF | |
| peptide | PAGDHVYPGLKTELHSMRSTLESIYKDMWQCPLLGCGDKSVISRLSQEA | |
| | ERKSDNGTRKGLSELDTLFSRLEEYLHSRK | |
| hIL-10R BP-110 | MLSVMVSSSLVLIVFFLGASEEAKPAATTTTIKNTKPQCRPEDYATRLQ | 110 |
| with signal | DERVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-111 | MLSVMVSSSLVLIVFFLGASEEAKPATIKNTKPQCRPEDYATRLQDLRV | 111 |
| with signal | TFHRVKPTLQREDDYSVWLDGTVVKGCWGCSVMDWLLRRYLEIVFPAGD | |
| peptide | HVYPGLKTELHSMRSTLESIYKDMRQCPLLGCGDKSVISRLSQEAERKS | |
| | DNGTRKGLSELDTLFSRLEEYLHSRK | |
| hIL-10R BP-112 | MLSVMVSSSLVLIVFFLGASEEAKPAATTTTTTTIKNTKPQCRPEDYAT | 112 |
| with signal | RLQDLRVTFHRVKPTLQREDDYSVWLDGTVVKGCWGCSVMDWLLRRYLE | |
| peptide | IVFPAGDHVYPGLKTELHSMRSTLESIYKDMRQCPLLGCGDKSVISRLS | |
| | QEAERKSDNGTRKGLSELDTLFSRLEEYLHSRK | |
| hIL-10R BP-113 | MLSVMVSSSLVLIVFFLGASEEAKPAATTTTTTMIKNTKPQCRPEDYAT | 113 |
| with signal | RLQDLRVTFHRVKPTLQREDDYSVWLDGTVVKGCWGCSVMDWLLRRYLE | |
| peptide | IVFPAGDHVYPGLKTELHSMRSTLESIYKDMRQCPLLGCGDKSVISRLS | |
| | QEAERKSDNGTRKGLSELDTLFSRLEEYLHSRK | |
| hIL-10R BP-114 | MLSVMVSSSLVLIVFFLGASEEAKPATTTTTIKNTKPQCRPEDYATRLQ | 114 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLRELDTLFSRLEEYLHSRK | |
| hIL-10R BP-115 | MLSVMVSSSLVLIVFFLGASEEAKPAATTTTTTTTIKNTKPQCRPEDYA | 115 |
| with signal | TRLQDLRVTFHRVKPTLQREDDYSVWLDGTVVKGCWGCSVMDWLLRRYL | |
| peptide | EIVFPAGDHVYPGLKTELHSMRSTLESIYKDMRQCPLLGCGDKSVISRL | |
| | SQEAERKSDNGTRKGLSELDTLFSRLEEYLHSRK | |
| hIL-10R BP-116 | MLSVMVSSSLVLIVFFLGASEEAKPAATTTTTTIKNTKPQCRPEDYATR | 116 |
| with signal | LQDLRVTFHRVKPTLQREDDYSVWLDGTVVKGCWRCSVMDWLLRRYLEI | |
| peptide | VFPAGDHVYPGLKTELHSMRSTLESIYKDMRQCPLLGCGDKSVISRLSQ | |
| | EAERKSDNGTRKGLSELDTLFSRLEEYLHSRK | |
| hIL-10R BP-117 | MLSVMVSSSLVMIVFFLGASEEAKPATTTTTIKNTKPQCRPEDYATRLQ | 117 |
| with signal | DLCVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-118 | MLSVMVSSSLVLIVFFLGASEEAKPAAATTTTTTTTIKNTKPQCRPEDY | 118 |
| with signal | ATRLQDLRVTFHRVKPTLQREDDYSVWLDGTVVKGCWGCSVMDWLLRRY | |
| peptide | LEIVFPAGDHVYPGLKTELHSMRSTLESIYKDMRQCPLLGCGDKSVISR | |
| | LSQEAERKSDNGTRKGLSELDTLFSRLEEYLHSRK | |
| hIL-10R BP-119 | MLSVMVSSSLVLIVFFLGASEEAKPAATTTTTTTIKNTKPQCRPEDYAT | 119 |
| with signal | RLQDLRVTFHRVKPTLQREDDYSVWLDGTVVKGCWGCSVMDWLLRRYLE | |
| peptide | IVFPAGDHVYPGLKTELHSMRSTLESIYKDMRQCPLLGCGDKSVISRLS | |
| | QEAERKSDNGTRKGLSELDTLFIRLEEYLHSRK | |
| hIL-10R BP-120 | MLSVMVSSSLVLIVFFLGASEEAKPATTTTTIKNTKPQCRPEDYATRLQ | 120 |
| with signal | DLRVTFHRVKPTLQREDDYSVWLDGTVVKGCWGRSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-121 | MLSVMVSSSLVLIVFFLGASEEAKPATTTTTIKNTKPQCRPEDYATRLQ | 121 |
| with signal | DLRVTFHRVKPTLQREDDYSVWLDGTVVKGCWGCSVMDWLLRRYLEIVE | |
| peptide | PAGDHVYPGLKTELHSMRSTLESICKDMRQRPLLGCGDKSVISRLSQEA | |
| | ERKSDNGTRKGLSELDTLFSRLEEYLHSRK | |
| hIL-10R BP-122 | MLSVMVSSSLVLIVFFLGASEEAKPAATTTTTTTTTIKNTKPQCRPEDY | 122 |
| with signal | ATRLQDFRVTFHRVKPTLQREDDYSVWLDGTVVKGCWGCSVMDWLLRRY | |
| peptide | LEIVFPAGDHVYPGLKTELHSMRSTLESIYKDMRQCPLLGCGDKSVISR | |
| | LSQEAERKSDNGTRKGLSELDTLFSRLEEYLHSRK | |
| hIL-10R BP-123 | MLSVMVSSSLVLIVFFLGASEEAKPATIKNTKPQCRPEDYATRLQDLRV | 123 |
| with signal | TFHRVKPTLPGHQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | RKSDNGTRKGLSELDTLFSRLEEYLHSRK | |
| hIL-10R BP-124 | MKTNTKIILFCYVILSLYVESCAIASAKKCDDVSEDYILKDLRSEFSKI | 124 |
| with signal | KSFVQDNDQENMMLLSQSMLDKLTSRIGCKSLSDMIKFYLNDVLPNAEK | |
| peptide | IEHMKNKITSIGEKLKSLKEKLISCDFLHCENHDEIKTVKTIENKLKDK | |
| | GIYKAMGEFDIFINYLEKYIVKK | |
| hIL-10R BP-125 | MKTNTKIILFCYVILSLYVESCAIASAKKCNDVSFDYILKDLRSEFSKI | 125 |
| with signal | KSFVQDNDQENMMLLSQSMLDKLTSRIGCKSLSDMIKFYLNDVLPNAEK | |
| peptide | IEHMKNKITSIGEKLKSLKEKLISCDFLHCENHDEIKTVKTIENKLKDK | |
| | GIYKAMGEFDIFINYLEKYIVKK | |
| hIL-10R BP-126 | MKTNTKIILFCYVIFLSLYVESCVVASAKKCDDVSFDYILKDLRSEFSK | 126 |
| with signal | IKSFVQDNDQENMMLLSQSMLDKLTSRIGCKSLSDMIKFYLNDVLPNAE | |
| peptide | KIEHMKNKITSIGEKLKSLKEKLISCDFLHCENHDEIKTVKTIENKLKD | |
| | KGIYKAMGEFDIFINYLEKYIVKK | |
| hIL-10R BP-127 | MKTNTKIILFCYVILSLYVESCVVAYAKKCDDVSEDYILKDLRSEFSKI | 127 |
| with signal | KSFVQNNDQENMMLLSQSMLNKLTSCIGCKSLSDMIKFYLNDVLPNAEK | |
| peptide | IEQIKNIITSIGEKLKSLKEKLISCDFLHCENNDEIKTVKAIFNKLKDK | |
| | GIYKAMGEFDIFINYVEKYIVKT | |
| hIL-10R BP-128 | MKTSTKIILFCYVILSLYVFSCVVASAKKCDDVSFDYILKDLRSEFIKI | 128 |
| with signal | KSFVQNNDQENMMLLSQSMLDKLTSCIGCKSLSDMIKFYLNDVLPNAEK | |
| peptide | IEQIKNIITSIGEKLKSLKEKLISCDFLHCENNDEIKTVKAIFNKLKDK | |
| | GIYKAMGEFDIFINYVEKYIVKT | |
| hIL-10R BP-129 | MKTNTKIILFCYVILYVFSCTVASAKKCDDVSFDYILKDLRSEFSKIKS | 129 |
| with signal | FVQNNDKENMMLLSQSMLDKLTSCIGCKSLSDMIKFYLNDVLPNAEKIE | |
| peptide | HIKNKITSIGEKLKSLKEKLISCDFLHCENHDEIKAVKTIFNKLKDKGI | |
| | YKAMGEFDIFINHLEKYIVKK | |
| hIL-10R BP-130 | MKTSTKIILFCYVILSLYVFSCVVASAKKCDDVSFDYILKDLRSEFIKI | 130 |
| with signal | KSFVQNNDQENMMLLSQSMLDKLTSRIGCKSLSDMIKFYLNDVLPNAEK | |
| peptide | IEQIKNIITSIGEKLKSLKEKLISCDELHCENNDEIKTVKAIFNKLKDK | |
| | GIYKAMGEFDIFINYVEKYIVKT | |
| hIL-10R BP-131 | MKTNTKIILFCYVILYLFSCTVASAKKCDDVSEDYILKDLRSEFSKIKS | 131 |
| with signal | FVQNNDKENMMLLSQSMLDKLTSCIGCKSLSDMIKFYLNDVLPNAEKIE | |
| peptide | HIKNKITSIGEKLKSLKEKLISCDFLHCENHDEIKAVKTIFNKLKDKGI | |
| | YKAMGEFDIFINHLEKYIVKK | |
| hIL-10R BP-132 | MLSVMVSSSLVLIVFFLGASEEAKPATTTTTIKNTKPQCRPEDYATRLQ | 132 |
| with signal | DLRVTFHRVKPTLDDYSVWLDGTVVKGCWGCSVMDWLLRRYLEIVFPAG | |
| peptide | DHVYPGLKTELHSMRSTLESIYKDMRQCPLLGCGDKAVISRLSQEAERK | |
| | SDNGTRKGLSELDTLFSRLEEYLHSRK | |
| hIL-10R BP-133 | MKTNTKIILFCYVILYLFSCTVASAKKCDDVSEDYILKDLRSEFSKIKS | 133 |
| with signal | FVQNNDKENMMLLSQSMLDKLTRCIGCKSLSDMIKFYLNDVLPNAEKIE | |
| peptide | HIKNKITSIGEKLKSLKERLISCDFLHCENHDEIKAVKTIFNELKDKGI | |
| | YKAMGEFDIFINHLEKYIVKK | |
| hIL-10R BP-134 | MERRLVVTLQCLVLLYLAPECGGTDQCDNFPQMLRDLRDAFSRVKTFFQ | 134 |
| with signal | TKDEVDNLLLKESLLEDFKGYLGCQALSEMIQFYLEEVMPQAENQDPEA | |
| peptide | KDHVNSLGENLKTLRLRLRRCHRFLPCENKSKAVEQIKMPLTSCRKKEF | |
| | TKP | |
| hIL-10R BP-135 | MGSRRLSRCSLATAVCLVAIVVAVAAKGRDSKPSPACDPMHGALAGIFK | 135 |
| with signal | ELRTTYRSVRETLQTKDTVYYVSLFHEQLLQEMLSPVGCRVTNELMQHY | |
| peptide | LDGVLPRAFHCGYDNTTLNALHELSSSLSTLYQHMLKCPALACTGQTPA | |
| | WTQFLDTEHKLDPWKGTVKATAEMDLLLNYLETELLQS | |
| hIL-10R BP-136 | MLSVMVSSSLVLIVFLLGASEEAKPATTTIKNTKPQCRPEDYATRLQDL | 136 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCEAERKSDNGTRKGLSELDTL | |
| | FSRLEEYLHSRK | |
| hIL-10R BP-137 | MRRRRSFGVVVVGAIGTLLMMAVVVLSAHDHEHKVPPACDPVHGNLAGI | 137 |
| with signal | FKELRTIYTSIREGLQKKDTVYYTSLENDRVLQEMLSPMGCRVTNEIME | |
| peptide | HYLDGVLPRASHLDYDNSTLNGLHAFASSMQALYQHMLKCPALACTGKT | |
| | PAWMYFLEVEHKLNPWRGTAKAAAEADLLLNYLETFLLQF | |
| hIL-10R BP-138 | MGSRPARMCGLSNLLCLLLVVLVAVVIHRGCGASKPPVDCDPIHGTLSR | 138 |
| with signal | IIKEVRTGYGSIKQALQSKDTVYYVSLFHENLLNEMLSPVGCRVTNELM | |
| peptide | QHYLDGVLPRAFQCGYDNTTLDGLHSLVSSLDALYKHMLKCPALACTGQ | |
| | TPAWTQFLETEHKLDPWKGTIKATAEMDLLVNYLETFLAQS | |
| hIL-10R BP-139 | MLSMMVSSSLVLIVFFLGASEEAKPATTTTTIKNTKPQCRPEDYATRLQ | 139 |
| with signal | DLRVTFHRVKPTLQREDDYSVWLDGTVVKGCWGCSVMDWLLRRYLEIVE | |
| peptide | PAGDHVYPGLKTELHSMRSTLESIYKDMRQCPLLGCGDKSVISR | |
| hIL-10R BP-140 | MLSVMVSSSLVLIVFFLGASEEAKPATTTIKNTKPQCRPEDYATRLQDL | 140 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCVSVSVAALSAQR | |
| hIL-10R BP-141 | MININILSLLILILSIYANAIIDTCYDDQERERTKSNSISSVTPEMCKG | 141 |
| with signal | LKQLVSTKLKDARQKEKSVRDYFTSRDNDLDEMLLQGVKETHKKTCGCY | |
| peptide | VLYLLLSFYGKTIRDTIQSNKHKNLNTELTNLAVSVLSLEDLLEACGIT | |
| | CNPKKDSLLKRIEEYMKEHGDDAIYKVIGEIEFLFQAIEKHVY | |
| hIL-10R BP-142 | MINISINILSLLILILSIYANSIIDMCYDDQERERTKSNSISSITPDMC | 142 |
| with signal | KGLKQLVATKLKDARQKEKLVNSYFTSRDNDLTYMLLQGVRETHKKPCG | |
| peptide | CYVLYLLLTFYRKTIKDTIQSKKHESINTELTNLAVTVLSLEDLLEACG | |
| | ITCNPKKDSLLKRIEGYTKEHGDDAIYKVIGEIDELFQAIERHVY | |
| hIL-10R BP-143 | MININILSLLILILSIYANAIIDTCYDDQERERTKSNSISSVTPEMCKG | 143 |
| with signal | LKQLVATKLKDARQKEKLVNDYFTGRDNDLSYMLLQGVRETHKKPCGCY | |
| peptide | VLYLLLSFYRKTIRDTIQSNKHASINAELTNLAVSVLSLEDLLDACGIT | |
| | CNPKKDSLLKRIEEYMKEHGDDAIYKLIGEIEFLFQAIERHVYT | |
| hIL-10R BP-144 | MININILSLLILILSIYANAIIDTYDEDEDEDSIKLSSIGSITPEMCKN | 144 |
| with signal | LKQLVASKLKDIRQKEKSLRDYFTNLDDELDYMLLQGVGENHKKKCGCY | |
| peptide | ILHLLLKFYSKTIRNTIQSEKHKNVNLELTNIAVSMLALEDLLEKCGIT | |
| | CNPKKDPLLKRIEDYMKQHGDDGVNKAIAELEFLFQMIEKQVYI | |
| hIL-10R BP-145 | MARFIYVVLLCLVEDAAQSAAQCRKGTITSRLKMLRTAFEKVREFYEDR | 145 |
| with signal | DEEETALASTEHLHGPESCSVIDELITHYTKCVIPAANEEEGADLLSLD | |
| peptide | TLQVALENVKGLLANCQEEFGCKPPFSMRDYKKQYRQLNKEKNAGMIKA | |
| | MGELGMLFNGIEERVIGM | |
| hIL-10R BP-146 | MMLALAMMLMALGPLSTNAMYVQHGSDYCTTTVHADIASAISGMRAEYD | 146 |
| with signal | SGLGHYFKSLVPHPDNPYDTDDYKYMINNTNSYNCHALQSTINALLGMY | |
| peptide | GYVDIDEPHQLAMMKLATHTMQAAMLLNKCAKQLGCYHIPEDVETLHEA | |
| | HPDDVMASLDTALNLMSMVTNEI | |
| hIL-10R BP-147 | MARFIYVVLLCLVEDAAQSAAQCRKGTITSRLKMLRTAFEKVREFYEDS | 147 |
| with signal | DEEETALASTEHLHGPESCSVIDELITHYTKCVIPAANEEEGADLLSLD | |
| peptide | TLQVALENVKGLLANCQEEFGCKPPFSMRDYKKQYRQLNKEKNAGMIKA | |
| | MGELGMLFNGIEERVIGM | |
| hIL-10R BP-148 | MFQLEGIVLLVYLANWVSPATIKCVGMSTLFNPELIQLRRLFGDGIKDE | 148 |
| with signal | FQNKDEDLDNAFLNEDVQRELASDCGCDHLMDMLSLYVNDTIPKGMKTE | |
| peptide | DAPSGLGQMGQLMSSLYRKMDMCWSELGCSHNTRLTLQEYADKKGGWDN | |
| | KALGESDILFDALELFFSKIK | |
| hIL-10R BP-149 | MMTTLALVMTLMALSTNAMYVQHGSDYCTTTVRADIASAISGMRAEYNN | 149 |
| with signal | GLGDYFKSLAPHPNNPYDTDDYKYMINSTNSYNCHALQSTINALLGMYG | |
| peptide | YVDIDEPHQLAMMKLATHTMQTAMLLNKCAAQLGCYHIPEDVETLHEAH | |
| | PNDVMASLDTALNLMSMVTNEI | |
| hIL-10R BP-150 | MITLALVMTLMALGPLSTNAMYARRSGDYCTTTVRADIASAISGMRAEY | 150 |
| with signal | NSGLRDYFKSLVPHPDNPYDTDDYKYMLNNTNSYNCHALQSTINALLGM | |
| peptide | YGYVDIDESHQLAMMKLATHTMQTAMMLNKCAAQLGCYHIPEDLETLHE | |
| | AHPDDVMASLDTALNLMSMITNEI | |
| hIL-10R BP-151 | MMTTLALVMTLMALATNAMYVQHGSDYCTTTVRADIASAISGMRAEYNN | 151 |
| with signal | GLGDYFKSLAPHPNNPYDTDDYKYMINSTNSYNCHALQSTINALLGMYG | |
| peptide | YVDIDEPHQLAMMKLATHTMQTAMLLNKCAAQLGCYHIPEDVETLHEAH | |
| | PDDVMASLDTALNLMSMVTNEI | |
| hIL-10R BP-152 | MSGTSNKKFVFLVAIAVAICMMSSVSSNVHSGTEDNPCTNSKTVLNTLL | 152 |
| with signal | NQIKQEYINNLLPYYKALTPKPVDVEDDSYTYSIQSTDYNCYTIYETLN | |
| peptide | FLLGDVFPRATTDATVRLSLAKIATSSQQASMLMNLCKKELACGPAPED | |
| | MIKLYHDTKEYGADNIMGTLDTPFQYFVIV | |
| hIL-10R BP-153 | MLALAMVLMALGPLSTNAMYVQHGRGDYCTTTVRADIASAISGMRAEYD | 153 |
| with signal | SGLGHYFKSLVPHPDNPYDTDDYKYMINNTNSYNCHALQSTINALLGMY | |
| peptide | GYVDIDEPHQLAMMKLATHTMQTAMLLNKCAEQLGCYHIPEDVEILHEA | |
| | HPDDVMASLDTALNLMSMVTNEI | |
| hIL-10R BP-154 | MFLAVLLTATIFFEARGAPATTPKDSCVYLIGQTPQLLRQLRNAYQAII | 154 |
| with signal | GADGSGVDEDDMPIYPSDVMNELASTSVACDAIKKVLTMNIGILPNVTA | |
| peptide | AYPDKKSEVDEIGDNLSRLHQNIVNCRDFLKCEDLPHWHQMAENYKEKP | |
| | MQGFSEMDFVFQSVEKFLVAKDVKNMKTKRKH | |
| hIL-10R BP-155 | MLKQIIVVCIVAMAAVFADDDPCTNVKTQLNTLENQIKTEYDTNLKTYY | 155 |
| with signal | QSIAPSAFDPFNNTNYLYSVQGNDYKCYTIFETLSFLMGDVYPRATTNE | |
| peptide | SVRLSLAKVATSSTQGAMVMNLCRQQLGCGPPPFDAKTLYDDRAEYGAD | |
| | DIMATLDTALAKFKLVLESENVV | |
| hIL-10R BP-156 | MLKQIIVVCIVAMAAVFADDDPCTNVKTQLNTLFNQIKTEYDTNLKTYY | 156 |
| with signal | QSIAPSAFDPFNNTNYLYSVQGNDYKCYTIFETLSELMGDVYPRATTNE | |
| peptide | SVRLSLAKVATSSTQGAMVMNLCREQLGCGPPPFDAKTLYDDRAEYGAD | |
| | DIMATLDTALAKFKLVLESENVV | |
| hIL-10R BP-157 | MITLALVMTLMALGPLSTNGVHARRRGDYCTTTVRADIASAISGMRAEY | 157 |
| with signal | NSGLGDYFKSLVPHPDNPYDTDDYKHMIDNANSYNCHALQSTINALLGM | |
| peptide | YGYVDIDEPHQLAMMKLATHTMQTAMLLNKCAAQLGCYHIPEDLETLRE | |
| | APPADVMASLDTALNLMSMITNEI | |
| hIL-10R BP-158 | MDAQFLLLIVLALPASFAASLSTHYNNYDLTRIATIDKDVCKRVAQHIN | 158 |
| with signal | DDFVNMRKLYETQLKNYFQQLVPNPTDVFKDDSYMYMINGTDYNCHIIY | |
| peptide | ETMRFLSGDVFPFATETEAELQYMWKMMLGVSQLSAYIGNCYQYFKCGP | |
| | APFDPQVLYHDRELFHADTVMAYLDTAFSHETL | |
| hIL-10R BP-159 | MKLYFYCIFFYKIIVTISLNCGIEHNELNNIKNIFFKVRNVVQADDVDH | 159 |
| with signal | NLRILTPALLNNITVSETCFFIYDMFELYLNDVFVKYTNTALKLNILKS | |
| peptide | LSSVANNFLAIFNKVKKRRVKKNTVNVLEIKKLLLIDNNCKKLESEIDI | |
| | FLTWVMAKI | |
| hIL-10R BP-160 | MKLYFYCIFFYKIIVTISLNCGIEHNELNNIKNIFFKVRNVVQADDVDH | 160 |
| with signal | NLRILTPALLNNITVSETCFFIYDMFELYLNDVFVKYTNTALKLNILKS | |
| peptide | LSSVANNFLAIFNKVKKRRVKKNNVNVLEIKKLLLIDNNCKKLFSEIDI | |
| | FLTWVMAKI | |
| hIL-10R BP-161 | MFLAVLLTATIFFEARGAPATTPKDSCVYLIGQTPQLLRQLRNAYQAII | 161 |
| with signal | GADGSGVDEDDMPIYPSDVMNELASTSVACDAIKKVLTMNIGILPNVTA | |
| peptide | AYPDKKSEVDEIGDNLSRLHQNIVNCVSRTQHLCYD | |
| hIL-10R BP-162 | MFRASLLCCLVLLAGVWADNKYDSESGDDCPTLPTSLPHMLHELRAAFS | 162 |
| with signal | RVKTFFQMKDQLDNMLLDGSLLEDFKGYLGCQALSEMIQFYLEEVMPQA | |
| peptide | ENHSPDQDKNKVNSLGEKLKTLRVRLRRCHRFLPCENKSKAVEQVKSAF | |
| | SKLQEKGVYKAMSEFDIFINYIEAYMTTKMKN | |
| hIL-10R BP-163 | MFRASLLCCLVLLAGVWADNKYDSESGNDCPTLPTSLPHMLHELRAAFS | 163 |
| with signal | RVKTFFQMKDQLDNMLLDGSLLEDFKGYLGCQALSEMIQFYLEEVMPQA | |
| peptide | ENHSTGQEKDKVNSLGEKLKTLRVRLRRCHRFLPCENKSKAVEQVKSAF | |
| | SKLQEKGVYKAMSEFDIFINYIEAYMTTKMKN | |
| hIL-10R BP-164 | MFRASLLCCLVLLAGVWADNKYDSESGNDCPTLPTSLPHMLHELRAAFS | 164 |
| with signal | RVKTFFQMKDQLDNMLLDGSLLEDFKGYLGCQALSEMIQFYLEEVMPQA | |
| peptide | ENHSTDQEKDKVNSLGEKLKTLRVRLRRCHRFLPCENKSKAVEQVKSAF | |
| | SKLQEKGVYKAMSEFDIFINYIEAYMTTKMKN | |
| hIL-10R BP-165 | MFRASLLCCLVLLAGVWADNKYDSESGDDCPTLPTSLPHMLHELRAAFS | 165 |
| with signal | RVKTFFQMKDQLDNMLLDGSLLEDFKGYLGCQALSEMIQFYLEEVMPQA | |
| peptide | ENHSTGQEKDKVNSLGEKLKTLRVRLRRCHRFLPCENKSKAVEQVKSAF | |
| | SKLQEKGVYKAMSEFDIFINYIEAYMTTKMKN | |
| hIL-10R BP-166 | MFRALLLCCLALLAGVWADNRYDGQDGNDCPTLPTSLPHMLHELRAAFS | 166 |
| with signal | RVKTFFQMKDQLDNMLLDGSLLEDFKGYLGCQALSEMIQFYLEEVMPQA | |
| peptide | ENHSPDQDKNKVNSLGEKLKTLRVRLRRCHRFLPCENKSKAVEQVKSAF | |
| | SKLQEKGVYKAMSEFDIFINYIEAYMTTKMKN | |
| hIL-10R BP-167 | MLSVMVSSSLVLIVFFLGASEEAKPATTAIKNTKPQCRPEDYATRLQDL | 167 |
| with signal | RVTFHRVKPTLQREDDYSVWLDGTVVKGCWGCSVMDWLLRRYLEIVFPA | |
| peptide | GDHVYPGLKTELHSMRSTLESIYKDMRQCPLLGCGDKSVISRLSQEAER | |
| | KSDNGTRKGLSELDTLFSRLEEYLHSRK | |
| hIL-10R BP-168 | MLSVMVSSSLVLIVFFLGAFEEAKPATTTTIKNTKPQCRPEDYATRLQD | 168 |
| with signal | LRVTFDRVKPTLQREDDYSVWLDGTVVKGCWGCSVMDWLLRRYLEIVFP | |
| peptide | AGDHVYPGLKTELHSMRSTLESIYKDMRQCPLLGCGDKSVISRLSQEAE | |
| | KKSDNGTRKGLSELDTLFSRLEEYLHSRK | |
| hIL-10R BP-169 | MLSVMVSSSLVLIVFFLGASEEAKPATTTTTIKNTKPQCRPEDYATRLQ | 169 |
| with signal | DLRVTFYRVKPTLQREDDYSVWLDGTVVKGCWGCSVMDWLLRRYLEIVE | |
| peptide | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-170 | MLSVMVSSSLVLIVFFLGASEEAKPATTTTTIKNTKPQCRPEDYATRLQ | 170 |
| with signal | DLRVTFHRVKPTLDDYSVWLDGTVVKGCWGCSVMDWLLRRYLEIVFPAG | |
| peptide | DHVYPGLKTELHSMRSTLESIYKDMRQCPLLGCGDKSVISRLSQEAERK | |
| | SDNGTRKGLSELDTLFSRLEEYLHSRK | |
| hIL-10R BP-171 | MKTNTKIILFCYVIFLSLYVFSCVVASTKKCDDVSFDYILKDLRSEFSK | 171 |
| with signal | IKSFVQDNDQENMMLLSQSMLDKLTSRIGCKSLSDMIKFYLNDVLPNAE | |
| peptide | KIEHMKNKITSIGEKLKSLKEKLISCDFLHCENHDEIKTVKTIFNKLKD | |
| | KGIYKAMGEFDIFINYLEKYIVKK | |
| hIL-10R BP-172 | MLSVMVSSSLVLIVFFLGASEEAKPATTTIKNTKPQCRPEDYATRLQDL | 172 |
| with signal | RVTFHRVKPTLQREDDYSVWLDGDHVYPGLKTELHSMRSTLESIYKDMR | |
| peptide | QCPLLGCGDKSVISRLSQEAERKSDNGTRKGLSELDTLFSRLEEYLHSR | |
| | K | |
| hIL-10R BP-173 | MSNFRILSLLIFSLIVHVNAMIGTCYDEDEEIERLKSNSISSITPGMCR | 173 |
| with signal | NLKHSVMIRLIDARQIEASIRSYFTDGDNNLSEMLLQGIREISKKKCGC | |
| peptide | YILNLMLRFYIQTIKHTILSNKHKDMNLELTNLAVTILSLESLLEKCGV | |
| | TCNPVKDPLLTRIEEYTRKHGDNAIYKTIGELEFLEDAIEKFV | |
| hIL-10R BP-174 | MARFIYVVLLCLVEDAAQSAAQCRKGTITIRLKMLRTAFEKVREFYEDR | 174 |
| with signal | DEEETALASTEHLHGPESCSVIDELITHYTKCVIPAANEEEGADLRSLD | |
| peptide | TLQFALENVKGLLANCQEEFGCKPPFSMRDYKKQYRQLNKEKNAGMIKA | |
| | MGELGMLFNGIEERVIGM | |
| hIL-10R BP-175 | MARFIYVVLLCLVEDAAQSAAQCRKGTITIRLKMLRTAFEKVREFYEDR | 175 |
| with signal | DEEETALASTEHLHGPESCSVIDELITHYTKCVIPAANEEEGADLRSLD | |
| peptide | TLQFALENVKGLLANCQEEFGCKPPFSMRDYKKQYRQLNKEKNAGMIKA | |
| | MGELGMLFNGIEERVNGM | |
| hIL-10R BP-176 | MARFIYVVLLCLVEDAAQSAAQCRKGTITIRLKMLRTAFEKVREFYEDR | 176 |
| with signal | DEEETALASTEHLHGPESCSVIDELITHYTKCVIPAANEEEGADLLSLD | |
| peptide | TLQFALENVKGLLANCQEEFGCKPPFSMRDYKKQYRQLNKEKNAGMIKA | |
| | MGELGMLFNGIEERVIGM | |
| hIL-10R BP-177 | MARFIYVVLLCLVEDAAQSAAQCRKGTITSRLKMLRTAFEKVREFYEDR | 177 |
| with signal | DEEETALASTEHLHGPESCSVIDELITHHTKCVIPAANEEEGADLLSLD | |
| peptide | TLQVALENVKGLLANCQEEFGCKPPFSMRDYKKQYRQLNKEKNAGMIKA | |
| | MGELGMLFNGIEERVIGM | |
| hIL-10R BP-178 | MLSVMVSSSLVLIVFLLGASEEAKPATTTIKNTKPQCRPEDYATRLQDL | 178 |
| with signal | RVTFHRVKPTLVGHVGDHVYPGLKTELHSMRSTLESIYKDMRQCEAERK | |
| peptide | SDNGTRKGLSELDTLFSRLEEYLHSRK | |
| hIL-10R BP-1 | SPGQGTQSENSCTHFPGNLPNMLRDLRDAFSRVKTFFQMKDQLDNLLLK | 179 |
| without signal | ESLLEDFKGYLGCQALSEMIQFYLEEVMPQAENQDPDIKAHVNSLGENL | |
| peptide | KTLRLRLRRCHRFLPCENKSKAVEQVKNAFNKLQEKGIYKAMSEFDIFI | |
| | NYIEAYMTMKIRN | |
| hIL-10R BP-2 | SPGQGTQSENSCTHFPGYLPNMLRDLRDAFSRVKTFFQMKDQLDNLLLK | 180 |
| without signal | ESLLEDFKGYLGCQALSEMIQFYLEEVMPQAENQDPDIKAHVQSLGENL | |
| peptide | KDLRLWLRRCHRFLPCENKSKAVEQVKNAFNKLQEKGIYKAMSEFDIFI | |
| | NYIEAYMTMKIRN | |
| hIL-10R BP-3 | SPGQGTQSENSCTHFPGNLPNMLRALRDAFSRVKTFFQMKDQLDNLLLK | 181 |
| without signal | ESLLEDFKGYLGCQALSEMIQFYLEEVMPQAENQDPDIKAHVNSLGENL | |
| peptide | KTLRLRLRRCHRFLPCENKSKAVEQVKNAFNKLQEKGIYKAMSEFDIFI | |
| | NYIEAYMTMKIRN | |
| hIL-10R BP-4 | RHCMFGDSLRNSPDMKNMLQDLRGGYSGSGIKRTFQGKDTLDSMLLTQS | 182 |
| without signal | LLDDFKGYLGCQALSEMIQFYLEEVMPQAENHGPTDSVKQLGEKLHTLN | |
| peptide | QKFGECPRWFPCYYNTTPAVENVKSVESKLQERGVYKAMSEFDIFINYI | |
| | ETYTTMK | |
| hIL-10R BP-5 | CQLESGEALPLGSRSADSRSVDGQRVPAPQNNYPGLLRDLRLGYEGFKQ | 183 |
| without signal | KVTDSHPDETLLGSSRLAGDLKGPLRCQALSEMIQFLLQVVLPDAENSR | |
| peptide | QDLRSQFSTLGDRITGLRQQLRRDPTVFPCESRSDGVSDLRSAYTRLGS | |
| | TGAEKVLSEFDIFINYIEAYVTSV | |
| hIL-10R BP-6 | YCVEYAESDEDRQQCSSSSNEPASLPHMLRELRAAFGKVKTFFQMKDQL | 184 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-7 | YCTSCSHHQCTEDENQKQDCEDANHSLPHMLRELRAAFGKVKTFFQMKD | 185 |
| without signal | QLHSLLLTQSLLDDFKGYLGCQALSEMIQFYLEEVMPQAENHGPEEHDN | |
| peptide | SLSEHGPDVKEHVNSLGEKLKTLRLRLRRCHRFLPCENKSKAVEKVKRV | |
| | FSELQERGVYKAMSEFDIFINYIETYMTT | |
| hIL-10R BP-8 | RSPKNKPSIDCNPQTGDFVNMLKSMRQDYSRIRDTLHDRDKLHSSLLTG | 186 |
| without signal | ALLDEMMGYSGCRTTLLLMEHYLDTWYPAAYRHHLYDNQTLVVVDRMGS | |
| peptide | TLVALLKAMVQCPMLACGAPSPAMDKMLQQEAKMKKYTGVYKGISETDL | |
| | LLGYLELYMMKEKR | |
| hIL-10R BP-9 | HEHKVPPACDPVHGNLAGIFKELRAIYASIREALQKKDTVYYTSLENDR | 187 |
| without signal | VLQEMLSPMGCRVTNELMEHYLDGVLPRAAHFDYDNSTLNGLHAFTSSM | |
| peptide | QALYQHMLKCPALACTGKTPAWMYFLEVEHKLNPWRGTAKAAAEADLLL | |
| | NYLETFLLQF | |
| hIL-10R BP-10 | KGRDSKPSPACDPMHGALAGIFKELRTTYRSVREALQTKDTVYYVSLFH | 188 |
| without signal | EQLLQEMLSPVGCRVTNELMQHYLDGVLPRAFHCGYDNATLNALHALSS | |
| peptide | SLSTLYQHMLKCPALACTGQTPAWTQFLDTEHKLDPWKGTVKATAEMDL | |
| | LLNYLETELLQS | |
| hIL-10R BP-11 | ATTTIKNTKPQCRPEDYATRLQDLRVTFHRVKPTLQREDDYSVWLDGTV | 189 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-15 | YCVEYEESEEDRQQCSSSNFPASLPHMLRELRAAFGKVKTFFQMKDQLN | 190 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKP | |
| hIL-10R BP-16 | YCVEYEESEEDRQQCSSSNFPASLPHMLRELRAAFGKVKTFFQMKDQLN | 191 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKT | |
| hIL-10R BP-17 | YCVEYEESEEDRQQCSSSNFPASLPHMLRELRAAFGKVKTFFQMKDQLN | 192 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKS | |
| hIL-10R BP-18 | YCVEYKESEEDRQQCSSSSFPASLPHMLRELRAAFGKVKTFFQMKDQLN | 193 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTK | |
| hIL-10R BP-19 | YCVEYAESDEDKQQCSGSNFPASLPHMLRELRAAFGKVKTFFQMKDQLN | 194 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKM | |
| hIL-10R BP-20 | YCVEYEESEEDKQQCGSNGGPASLPHMLRELRAAFGKVKTFFQMKDQLN | 195 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKM | |
| hIL-10R BP-21 | YCVEYAESDEDRQQCSGSNFPASLPHMLRELRAAFGKVKTFFQMKDQLN | 196 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKM | |
| hIL-10R BP-22 | YCVEYEESEEDKQQCSSSSNFPASLPHMLRELRAAFGKVKTFFQMKDQL | 197 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-23 | YCIQYEESEEDKQQCSSSNFPASLPHMLRELRAAFGKVKTFFQMKDQLN | 198 |
| without signal | SMLLTQSLLDDFKGYLGCQALSEMIRFYLEEVMPQAENHGPDIKEHVNS | |
| peptide | LGEKLKTLRLRLRRCHRFLPCENKSKAVEQVKRVFNMLQERGVYKAMSE | |
| | FDIFINYIESYMTTKM | |
| hIL-10R BP-24 | YCVEYEESEEDRQQCSSSNFPASLPHMLRELRAAFGKVKTFFQMKDQLN | 199 |
| without signal | SMLLTQSLLDDFKGYLGCQALSEMIQFYLEEVMPQAENHGPDIKEHVNS | |
| peptide | LREKLKTLRLRLRRCHRFLPCENKSKAVEQVKRVFNMLQERGVYKAMSE | |
| | FDIFINYIESYMTTKT | |
| hIL-10R BP-25 | YCVEYEESEEDKQQCGSSSNEPASLPHMLRELRAAFGKVKTFFQMKDQL | 200 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-26 | YCVEYAESDEDKQQCSGSNFPASLPHMLRELRAAFGKVKTFFQMKDQLN | 201 |
| without signal | SMLLTQSLLDDFKGYLGCQALSEMIQFYLEEVMPRAENHGPDIKEHVNS | |
| peptide | LGEKLKTLRLRLRRCHRFLPCENKSKAVEQVKRVFNMLQERGVYKAMSE | |
| | FDIFINYIESYMTTKM | |
| hIL-10R BP-27 | YCVEYEESEEDRQQCSGSSNFPASLPHMLRELRAAFGKVKTFFQMKDQL | 202 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-28 | YCVEYEESDEDRQQCSSSSNFPASLPHMLRELRAAFGKVKTFFQMKDQL | 203 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-29 | YCVEYEESDEDKQQCSSSTGAPASLPHMLRELRAAFGKVKTFFQMKDQL | 204 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-30 | YCVEYEETKEDEQQCSSSSNFPASLPHMLRELRAAFGKVKTFFQMKDQL | 205 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-31 | YCVEYEESEEDRQQCSSSNFPASLPHMLRELRAAFGKVKTFFQMKDQLN | 206 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGDYKAMSE | |
| | FDIFINYIESYMTTKS | |
| hIL-10R BP-32 | YCVEYEESEEDRQQCSSSSNFPASLPHMLRELRAAFGKVKTFFQMKDQL | 207 |
| without signal | NSMLLTQSLLDDFKGYLGCQALSEMIQFYLEEVMPQAENHGPDIKEHVN | |
| peptide | SLGEKLKTLRLRLRRCHRELPCEDKSKAVEQVKRVFNMLQERGVYKAMS | |
| | EFDILINYIESYMTTKM | |
| hIL-10R BP-33 | YCVEYEESDEDRQQCSSSSNFPASLPHMLRELRAAFGKVKTFFQMKDQL | 208 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMATKM | |
| hIL-10R BP-34 | DNRYDGQDGNDCPTLPTSLPHMLHELRAAFSRVKTFFQMKDQLDNMLLD | 209 |
| without signal | GSLLEDFKGYLGCQALSEMIQFYLEEVMPQAENHSTDQEKDKVNSLGEK | |
| peptide | LKTLRVRLRRCHRFLPCENKSKAVEQVKSAFSKLQEKGVYKAMSEFDIE | |
| | INYIEAYMTTKMKN | |
| hIL-10R BP-35 | YCVEYLESREDEQQCSSSSNFPASLPHMLRELRAAFGKVKTFFQMKDQL | 210 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-36 | YCVEYEESKEDEQQCSGSNGASASLPHMLRELRAAFGKVKTFFQMKDQL | 211 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-37 | YCVEYLESGEDEQQCGSSSNFPASLPHMLRELRAAFGKVKTFFQMKDQL | 212 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-38 | YCVEYLESREDEQQCSGSNGASASLPHMLRELRAAFGKVKTFFQMKDQL | 213 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-39 | ESENNCTHFPTSLPHMLHELRAAFSRVKTFFQMKDQLDNMLLNGSLLED | 214 |
| without signal | FKGYLGCQALSEMIQFYLEEVMPQAENHSGGGGPDIKEHVNSLGEKLKT | |
| peptide | LRVRLRRCHRFLPCENKSKAVEQVKSAFSKLQEKGVYKAMSEFDIFINY | |
| | IEAYMTTKMKNKK | |
| hIL-10R BP-40 | YCVEYLESDEDKQHCSSSNGASASSPHMLRELRAAFGKVKTFFQMKDQL | 215 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIESYMTTKM | |
| hIL-10R BP-41 | YCVEYLESEEDKQQCGSNGASSSSPHMLRELRAAFGKVKTFFQMKDQLN | 216 |
| without signal | SMLLTQSLLDDFKGYLGCQALSEMIQFYLEEVMPQAENHGPDVKEHVNS | |
| peptide | LAEKLKTLRLRLRRCHRFLPCENKSKAVEQVKRVFNMLQERGVYKAMSE | |
| | FDIFINYIESYMTTKS | |
| hIL-10R BP-42 | YCVEYLESEEDKQQCGSNGASSSSPHMLRELRAAFGKVKTFFQMKDQLN | 217 |
| without signal | SMLLTQSLLDDFKGYLGCQALSEMIQFYLEEVMPQAENHGPDVKEHVNS | |
| peptide | LGEKLKTLRLRLRRCHRFLPCENKSKAVAQVKRVFNMLQERGVYKAMSE | |
| | FDIFINYIESYMTTKS | |
| hIL-10R BP-43 | YCVEYEESEEDRQQCSSSNFPASLPHMLRELRAAFGKVKTFFQMKDQLN | 218 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIES | |
| hIL-10R BP-44 | YCVEYEESEEDRQQCSSSNFPASLPHMLRELRAAFGKVKTFFQMKDQLN | 219 |
| without signal | SMLLTQSLLDDFKGYLGCQAFSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIES | |
| hIL-10R BP-45 | YCVEYLESREDEQQCSSSSNEPASLPHMLRELRAAFGKVKTFFQMKDQL | 220 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIES | |
| hIL-10R BP-46 | VSNCGNLPHMLRDLRDAFSRVKTFFQMKDQLDNILLKESLLEDFKGYLG | 221 |
| without signal | CQALSEMIQFYLEEVMPQAENQDPHAKEHVNSLGENLKTLRLRLRRCHR | |
| peptide | FLPCENKSKAVEQVKNAFSKLQEKGVYKAMSEFDIFINYIEAYMTMKIR | |
| | R | |
| hIL-10R BP-47 | YCVEYLESREDEQQCGSSSNFPASLPHMLRELRAAFGKVKTFFQMKDQL | 222 |
| without signal | NSMLLTQSLLDDFKGYLGCQALSEMIQFYLEEVMPQAENHGPDIKEHVN | |
| peptide | SLGEKLKTLRLRLRRCHRFLPCENKSKAVEQVKRVFNMLQERGVYKAMS | |
| | EFDIFINYIES | |
| hIL-10R BP-48 | YCVEYEESEEDRQQCSSSNFPASLPHMLRELRAAFGKVKTFFQMKDQLN | 223 |
| without signal | SMLLTQSLLDDFKGYLGCQALSEMIQFYLEEVMPQAENHGPDIKEHVNS | |
| peptide | PGEKLKTLRLRLRRCHRFLPCENKSKAVEQVKRVFNMLQERGVYKAMSE | |
| | FDIFINYIES | |
| hIL-10R BP-49 | YCTSCSYRDCTEDEDQKQQCEGGLRSLPHMLRELRAAFGKVKTFFQMKD | 224 |
| without signal | QLHSLLLTQSLLDDFKGYLGCQALSEMIQFYLEEVMPQAENHGPDIKEH | |
| peptide | VNSLGEKLKTLRLRLRRCHRFLPCENKSKAVEKVKRVESELQERGVYKA | |
| | MSEFDIFINYIETYMT | |
| hIL-10R BP-50 | YCVEYEESEEDRQQCSSSNFPASLPHMPRELRAAFGKVKTFFQMKDQLN | 225 |
| without signal | SMLLTQSLLDDFKGYLGCQALSEMIQFYLEEVMPQAENHGPDIKEHVNS | |
| peptide | LGEKLKTLRLRLRRCHRFLPCENKSKAVEQVKRVFNMLQERGVYKAMSE | |
| | FDIFINYIES | |
| hIL-10R BP-51 | YCTSCSHRDCTEDDEQKQQCEGGSGGLGSLPHMLRELRAAFGKVKTFFQ | 226 |
| without signal | MKDQLHSLLLTQSLLDDFKGYLGCQALSEMIQFYLEEVMPQAENHGPDI | |
| peptide | KEHVNSLGEKLKTLRLRLRRCHRFLPCENKSKAVEKVKRVESELQERGV | |
| | YKAMSEFDIFINYIETYMT | |
| hIL-10R BP-52 | TDQCDNFPQMLRDLRDAFSRVKTFFQTKDEVDNLLLKESLLEDFKGYLG | 227 |
| without signal | CQALSEMIQFYLEEVMPQAENQDPGAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-53 | TDQCDNFPQMLRDLRDAFSRVKTFFQTKDEVDNLLLKESLLEDFKGYLG | 228 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-54 | TDQCDNFPQMLRDLRDAFSRVKTFFQTKDEVDNLLLKESLLEDFKGYLG | 229 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTMKAR | |
| hIL-10R BP-55 | VSNCGNLPHMLRDLRDAFSRVKTFFQMKDQLDNILLKESLLEDFKGYLG | 230 |
| without signal | CQALSEMIQFYLEEVMPQAENQDPNAKEHVNSLGENLKTLRLRLRRCHR | |
| peptide | FLPCENKSKAVEQVKNAFSKLQEKGVYKAMSEFDIFINYIEAYMTMKTR | |
| | R | |
| hIL-10R BP-56 | TDQCDNFPQMLRDLRDAFSRVKTFFQTKDEVDNLLLKESLLEDFKGYLG | 231 |
| without signal | CQALSEMIQFYLEEVMPQAENQDPEAKDHVNSLGENLKTIRLRLRRCHR | |
| peptide | FLPCENKSKAVEQIKNAFNKLQEKGIYKAMSEFDIFINYIEAYMTIKAR | |
| hIL-10R BP-57 | TDQCDNFPQMLRDLRDAFSRVKTFFQTKDEVDSLLLKESLLEDFKGYLG | 232 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-58 | TDQCDNFPQMLRDLRDAFSRVKTFFQTKDEVDNILLKESLLEDFKGYLG | 233 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-59 | TDQCDNFPQMLRDLRDAFSRVKTFFQTKDEVDNLELKESLLEDFKGYLG | 234 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-60 | TDQCDNFPQMLRDLRDAFSRVKTFFQTKDEVDNLLLKESLLEDFKGYLG | 235 |
| without signal | CQALSEMIQFYLEKVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-61 | TDQCDNFPQMLRDLRDAFSRVKTFFQTKDAVDNLLLKESLLEDFKGYLG | 236 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMTIKAR | |
| hIL-10R BP-62 | ASNCGNLPHMLRDLRDAFSRVKTFFQMKDQLDNILLKESLLEDERGYLG | 237 |
| without signal | CQALSEMIQFYLEEVMPQAENQDPHSKEHVNSLGENLKTLRLRLRRCHR | |
| peptide | FLPCENKGKAVEQVKNAFSKLQEKGVYKAMSEFDIFINYIEAYMTMKLR | |
| | R | |
| hIL-10R BP-63 | TDQCDNFPQMLRDLRDAFSRVKTFFQTKDEVDNLLLKESLLEDFKGYLG | 238 |
| without signal | CQALSEMIQFYLEEVMPQAENQDPEAKDHVNSLGENLKTLRLRLRRCHR | |
| peptide | FLPCENKSKAVEQIKNAFNKLQEKGIYKAMSEFHIFINYIEAYMTIKAR | |
| hIL-10R BP-64 | EMLRDLRDAFSRVKTFFQTKDEVDNLLLKESLLEDFKGYLGCQALSEMI | 239 |
| without signal | QFYLEEVMPQAENQDPEAKDHVNSLGENLKTLRLRLRRCHRFLPCENKS | |
| peptide | KAVEQIKNAFNKLQEKGIYKAMSEFDIFINYIEAYMTIKAR | |
| hIL-10R BP-65 | ETCGNIPHMLRDLRDAFSRVKTFFQMKDQLDNILLKESLLEDFKGYLGC | 240 |
| without signal | QALSEMIQFYLEEVMPQAEAMSLKSQEHVNELGENLNTLRLRLRRCHRE | |
| peptide | LPCENKSKAVEQVKNAFSKLQEKGVYKAMSEFDIFINYIEAYMTMKLRR | |
| hIL-10R BP-66 | TANNRAQKCFCFDGSNAGNSEETNTAAFQKKCDSEIPESLPYMLRDLRN | 241 |
| without signal | SSVQTRRYFQEKDEENSPLLTQKLLEDFKGYLGCQALSEMIQFYLEEVM | |
| peptide | PQAEDSNPSAKDSVTSLGEKLKTLRLRLRRCHRFLPCENKSKAVENLKS | |
| | KFGDLGNQGVHKAMSEFDIFINYIETYMTTKMK | |
| hIL-10R BP-67 | ATTTTIKNTKPQCRPEDYATRLQDLRVTFHRVKPTLQREDDYSVWLDGT | 242 |
| without signal | VVKGCWGCSVMDWLLRRYLETVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-68 | ATTTTIKNTKPQCRPEDYATRLQDLRVTFHRVKPTLQREDDYSVWLDGT | 243 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-69 | ATTTTKNTKPQCRPEDYATRLQDLRVTFHRVKPTLQREDDYSVWLDGTV | 244 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-70 | ATTTTIKNTKPQCRPEDYATRLQDLRVTFHRVKPTLQREDDYSVWLDGT | 245 |
| without signal | MVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-71 | ATTTTIKNTKPQCRPEDYATRLQDLRVTFHRVKSTLQREDDYSVWLDGT | 246 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-72 | ATTTTIKNTKPQCRPEDYATRLQDLRVTFHRVKPTLQREDDYSVWLDGT | 247 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDMLFSRLEEYL | |
| | HSRK | |
| hIL-10R BP-73 | ATTTTIKNTKPQCRPEDYATRLQDLRVTFHRVKPTLQREDDYSVWLDGT | 248 |
| without signal | VVKGCWGCSVMDWLLRRYLEIVLPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-74 | AATTTIKNTKPQCRPEDYATRLQDLRVTFHRVKPTLQREDDYSVWLDGT | 249 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-75 | ATTTTINNTKPQCRPEDYATRLQDLRVTFHRVKPTLQREDDYSVWLDGT | 250 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-76 | ATTTTIKNTKPQCRPEDYATRLQDLRVTFHRVKPTLQREDDYSVWLDGT | 251 |
| without signal | VVKGCWGCSVMDWLLRRYLEIVFPAGDHVYPGLKTELHSMHSTLESIYK | |
| peptide | DMRQCPLLGCGDKSVISRLSQEAERKSDNGTRKGLSELDTLFSRLEEYL | |
| | HSRK | |
| hIL-10R BP-77 | ATTTTIKNTKPQCRPEDYATRLQDLRVTFHRVKPTLQREDDYSVWLDGT | 252 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGMRKGLSELDTLFSRLEEYL | |
| | HSRK | |
| hIL-10R BP-78 | ATTTIKNTKPQCRPEDYATRLQDLRVTFHRVKPTLQREDDYSVWLDGTV | 253 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-79 | ATTTTIKNTKPQCRPEDYATRLQDLRVTFDRVKPTLQREDDYSVWLDGT | 254 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-80 | AATTTIKNTKPQCRPEDYATRLQDLRVTFHRVKPTLQHEDDYSVWLDGT | 255 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-81 | ATTTTIKNTKPQCRPEDYATRLQDLRVTFHRVKPTLQREDDYSVWLDGM | 256 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-82 | ATTTTIKNTKPQCRPEDYATRLQDLRVTFHRVKPTLQREDDYSVWLDGT | 257 |
| without signal | VVKGCWGCSVVDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-83 | ATTTTIKNTKPQCRPEDYATRLQDLRITFHRVKPTLQREDDYSVWLDGT | 258 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-84 | ATTTIKNTKPQCRPEDYATRLQDLRVTEHRIKPTLQREDDYSVWLDGTV | 259 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-85 | ATTTTIKNTKPQCRPEDYATRLQDLRVTFHRVKPTLQCEDDYSVWLDGT | 260 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-86 | ATTTTTIKNTKPQCRPEDYATRLQDLRVTFHRVKPTLQREDDYSVWLDG | 261 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-87 | ATTTTIKNTKPQCRPEDYATRLQDLRVTFHRVKPTLQREDDYSVWLDGT | 262 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDALFSRLEEYL | |
| | HSRK | |
| hIL-10R BP-88 | ATTTTIKNTKPQCRPEDYATRLQDLRVTFHRVKPTLQREDDYSVWLDGT | 263 |
| without signal | VVKGCWGCSVMDWLLRRYLEILFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-89 | ATTTTTIKNTKPQCRPEDYATRLQDLRVTFHRVKPTLQREDDYSVWLDG | 264 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGMRKGLSELDTLFSRLEEY | |
| | LHSRK | |
| hIL-10R BP-90 | ATTTIKNTKPQCRPEDYATRLQDLRVTFHRVKPTLQREDDYSVWLDGTV | 265 |
| without signal | VKGCWGCSVMDWLLRRYLEIVFPAGDHVYPRLKTELHSMRSTLESIYKD | |
| peptide | MQQCPLLGCGDKSVISRLSQEAERKSDNGTRKGLSELDTLFSRLEEYLH | |
| | SRK | |
| hIL-10R BP-91 | AATTTTIKNTKPQCRPEDYATRLQDLRVTFHRVKPTLQREDDYSVWLDG | 266 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-92 | AATTTTTIKNTKPQCRPEDYATRLQDLRVTFHRVKPTLQREDDYSVWLD | 267 |
| without signal | GTVVKGCWGCSVMDWLLRRYLEIVFPAGDHVYPGLKTELHSMRSTLESI | |
| peptide | YKDMRQCPLLGCGDKSVISRLSQEAEKKSDNGTRKGLSELDTLFSRLEE | |
| | YLHSRK | |
| hIL-10R BP-93 | AATTTTIKNTKPQCRPEDYASRLQDLRVTFHRVKPTLQREDDYSVWLDG | 268 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-94 | ATTTTTIKNTKPQCRPEDYATRLQDLRVTFHRVKPTLQREDDYSVWLDG | 269 |
| without signal | TVVKGCWGCSVMDWLLRRYLEIMFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-95 | ATTTIKNTKPRCRPEDYATRLQDLRVTFHRVKPTLQREDDYSVWLDGTV | 270 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-96 | ATTTTIKNTKPQCRPEDYATRLQDLRVTFHRVKPTLQREDDYSVWLDGT | 271 |
| without signal | VVKGCWGCSVMDWLLRRYLEIVFPAGDYVYPGLKTELHSMRSTLESIYK | |
| peptide | DMRQCPLLGCGDKSVISRLSQEAERKSDNGMRKGLSELDTLFSRLEEYL | |
| | HSRK | |
| hIL-10R BP-97 | ATTTIKNTKPQCRPEDYATRLQDLRVTFHRVKPTLQREDDYSVWLDGTV | 272 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDALFSRLEEYLH | |
| | SRK | |
| hIL-10R BP-98 | ATTTTTIKNTKPRCRPEDYATRLQDLRVTFHRVKPTLQREDDYSVWLDG | 273 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-99 | AATTTTTIKNTKPQCRPEDYATRLQDLRVTFHRVKPTLQREDDYSVWLD | 274 |
| without signal | GTVVKGCWGCSVMDWLLRRYLEIVFPAGDHVYPGLKTELHSMRSTLESI | |
| peptide | YKDMRQCPLLGCGDKSVISRLSQEAERKSDNGTRKGLSELDTLFSRLEE | |
| | YLHSRK | |
| hIL-10R BP-100 | ATTTTTTIKNTKPQCRPEDYATRLQDLRVTFHRVKPTLQREDDYSVWLD | 275 |
| without signal | GTVVKGCWGCSVMDWLLRRYLEIVFPAGDHVYPGLKTELHSMRSTLESI | |
| peptide | YKDMRQCPLLGCGDKSVISRLSQEAERKSDNGTRKGLSELDTLFSRLEE | |
| | YLHSRK | |
| hIL-10R BP-101 | AATTTIKNTKPQCRPEDYATRLQDLRVTFHRVKPTLQREDDYSVWLDGT | 276 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLNELDTLFSRLEEYL | |
| | HSRK | |
| hIL-10R BP-102 | ATTTTTIKNTKPQCRPEDYATRLQDLRVTFHRVKPTLQREDDYSVWLDG | 277 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDALFSRLEEY | |
| | LHSRK | |
| hIL-10R BP-103 | ATTTTIKNTKPQCRPEDYATRLQDLCVTFHRVKPTLQREDDYSVWLDGT | 278 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-104 | ATTTTTTTIKNTKPQCRPEDYATRLQDLRVTFHRVKPTLQREDDYSVWL | 279 |
| without signal | DGTVVKGCWGCSVMDWLLRRYLEIVFPAGDHVYPGLKTELHSMRSTLES | |
| peptide | IYKDMRQCPLLGCGDKSVISRLSQKAERKSDNGTRKGLSELDTLFSRLE | |
| | EYLHSRK | |
| hIL-10R BP-105 | AATTTTIKNTKPQCRPEDYASRLQDLRVTFHRVKPTLQREDDYSVWLDG | 280 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKSLSELDTLFSRLEEY | |
| | LHSRK | |
| hIL-10R BP-106 | ATTTIKNTKPQCRPEDYATRLQDLRVTFHRVKPTLQREDDYSVWLDGTV | 281 |
| without signal | VKGCWGCSVMDWLLRRYLEIVFPAGDHVYSGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-107 | ATTTTTTIKNTKPQCRPEDYATRLQDLRVTFHRVKPTLQREDDYSVWLD | 282 |
| without signal | GTVVKGCWGCSVMDWLLRRYLEIVFPAGDHVYPGLKTELHSMRSTLESI | |
| peptide | YKDMRQCPLLGCGDKSVISRLSQKAERKSDNGTRKGLSELDTLFSRLEE | |
| | YLHSRK | |
| hIL-10R BP-108 | ATTTTTMIKNTKPQCRPEDYATRLQDLRVTFHRVKPTLQREDDYSVWLD | 283 |
| without signal | GTVVKGCWGCSVMDWLLRRYLEIVFPAGDHVYPGLKTELHSMRSTLESI | |
| peptide | YKDMRQCPLLGCGDKSVISRLSQEAERKSDNGTRKGLSELDTLFSRLEE | |
| | YLHSRK | |
| hIL-10R BP-109 | ATTTTTIKNTKPQCRPEDYATRLQDLRVTFHRVKPTLQREDDYSVWLDG | 284 |
| without signal | TVVKGCWGCSVMDWLLRRYLEIVFPAGDHVYPGLKTELHSMRSTLESIY | |
| peptide | KDMWQCPLLGCGDKSVISRLSQEAERKSDNGTRKGLSELDTLFSRLEEY | |
| | LHSRK | |
| hIL-10R BP-110 | AATTTTIKNTKPQCRPEDYATRLQDERVTFHRVKPTLQREDDYSVWLDG | 285 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-111 | ATIKNTKPQCRPEDYATRLQDLRVTFHRVKPTLQREDDYSVWLDGTVVK | 286 |
| without signal | GCWGCSVMDWLLRRYLEIVFPAGDHVYPGLKTELHSMRSTLESIYKDMR | |
| peptide | QCPLLGCGDKSVISRLSQEAERKSDNGTRKGLSELDTLFSRLEEYLHSR | |
| | K | |
| hIL-10R BP-112 | ATTTTTTTIKNTKPQCRPEDYATRLQDLRVTFHRVKPTLQREDDYSVWL | 287 |
| without signal | DGTVVKGCWGCSVMDWLLRRYLEIVFPAGDHVYPGLKTELHSMRSTLES | |
| peptide | IYKDMRQCPLLGCGDKSVISRLSQEAERKSDNGTRKGLSELDTLFSRLE | |
| | EYLHSRK | |
| hIL-10R BP-113 | ATTTTTTMIKNTKPQCRPEDYATRLQDLRVTFHRVKPTLQREDDYSVWL | 288 |
| without signal | DGTVVKGCWGCSVMDWLLRRYLEIVFPAGDHVYPGLKTELHSMRSTLES | |
| peptide | IYKDMRQCPLLGCGDKSVISRLSQEAERKSDNGTRKGLSELDTLFSRLE | |
| | EYLHSRK | |
| hIL-10R BP-114 | ATTTTTIKNTKPQCRPEDYATRLQDLRVTFHRVKPTLQREDDYSVWLDG | 289 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLRELDTLFSRLEEY | |
| | LHSRK | |
| hIL-10R BP-115 | AKPAATTTTTTTTIKNTKPQCRPEDYATRLQDLRVTFHRVKPTLQREDD | 290 |
| without signal | YSVWLDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSELDTL | |
| | FSRLEEYLHSRK | |
| hIL-10R BP-116 | ATTTTTTIKNTKPQCRPEDYATRLQDLRVTFHRVKPTLQREDDYSVWLD | 291 |
| without signal | GTVVKGCWRCSVMDWLLRRYLEIVFPAGDHVYPGLKTELHSMRSTLESI | |
| peptide | YKDMRQCPLLGCGDKSVISRLSQEAERKSDNGTRKGLSELDTLFSRLEE | |
| | YLHSRK | |
| hIL-10R BP-117 | ATTTTTIKNTKPQCRPEDYATRLQDLCVTFHRVKPTLQREDDYSVWLDG | 292 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-118 | ATTTTTTTTIKNTKPQCRPEDYATRLQDLRVTFHRVKPTLQREDDYSVW | 293 |
| without signal | LDGTVVKGCWGCSVMDWLLRRYLEIVFPAGDHVYPGLKTELHSMRSTLE | |
| peptide | SIYKDMRQCPLLGCGDKSVISRLSQEAERKSDNGTRKGLSELDTLFSRL | |
| | EEYLHSRK | |
| hIL-10R BP-119 | ATTTTTTTIKNTKPQCRPEDYATRLQDLRVTFHRVKPTLQREDDYSVWL | 294 |
| without signal | DGTVVKGCWGCSVMDWLLRRYLEIVFPAGDHVYPGLKTELHSMRSTLES | |
| peptide | IYKDMRQCPLLGCGDKSVISRLSQEAERKSDNGTRKGLSELDTLFIRLE | |
| | EYLHSRK | |
| hIL-10R BP-120 | ATTTTTIKNTKPQCRPEDYATRLQDLRVTFHRVKPTLQREDDYSVWLDG | 295 |
| without signal | TVVKGCWGRSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-121 | ATTTTTIKNTKPQCRPEDYATRLQDLRVTFHRVKPTLQREDDYSVWLDG | 296 |
| without signal | TVVKGCWGCSVMDWLLRRYLEIVFPAGDHVYPGLKTELHSMRSTLESIC | |
| peptide | KDMRQRPLLGCGDKSVISRLSQEAERKSDNGTRKGLSELDTLFSRLEEY | |
| | LHSRK | |
| hIL-10R BP-122 | AKPAATTTTTTTTTIKNTKPQCRPEDYATRLQDERVTFHRVKPTLQRED | 297 |
| without signal | DYSVWLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLSELDT | |
| | LFSRLEEYLHSRK | |
| hIL-10R BP-123 | ATIKNTKPQCRPEDYATRLQDLRVTFHRVKPTLPGHQREDDYSVWLDGT | 298 |
| without signal | VVKGCWGCSVMDWLLRRYLEIVFPAGDHVYPGLKTELHSMRSTLESIYK | |
| peptide | DMRQCPLLGCGDKSVISRLSQEAERKSDNGTRKGLSELDTLFSRLEEYL | |
| | HSRK | |
| hIL-10R BP-124 | CAIASAKKCDDVSFDYILKDLRSEFSKIKSFVQDNDQENMMLLSQSMLD | 299 |
| without signal | KLTSRIGCKSLSDMIKFYLNDVLPNAEKIEHMKNKITSIGEKLKSLKEK | |
| peptide | LISCDFLHCENHDEIKTVKTIFNKLKDKGIYKAMGEFDIFINYLEKYIV | |
| | KK | |
| hIL-10R BP-125 | CAIASAKKCNDVSEDYILKDLRSEFSKIKSFVQDNDQENMMLLSQSMLD | 300 |
| without signal | KLTSRIGCKSLSDMIKFYLNDVLPNAEKIEHMKNKITSIGEKLKSLKEK | |
| peptide | LISCDFLHCENHDEIKTVKTIFNKLKDKGIYKAMGEFDIFINYLEKYIV | |
| | KK | |
| hIL-10R BP-126 | CVVASAKKCDDVSFDYILKDLRSEFSKIKSFVQDNDQENMMLLSQSMLD | 301 |
| without signal | KLTSRIGCKSLSDMIKFYLNDVLPNAEKIEHMKNKITSIGEKLKSLKEK | |
| peptide | LISCDFLHCENHDEIKTVKTIENKLKDKGIYKAMGEFDIFINYLEKYIV | |
| | KK | |
| hIL-10R BP-127 | CVVAYAKKCDDVSFDYILKDLRSEFSKIKSFVQNNDQENMMLLSQSMLN | 302 |
| without signal | KLTSCIGCKSLSDMIKFYLNDVLPNAEKIEQIKNIITSIGEKLKSLKEK | |
| peptide | LISCDFLHCENNDEIKTVKAIFNKLKDKGIYKAMGEFDIFINYVEKYIV | |
| | KT | |
| hIL-10R BP-128 | CVVASAKKCDDVSFDYILKDLRSEFIKIKSFVQNNDQENMMLLSQSMLD | 303 |
| without signal | KLTSCIGCKSLSDMIKFYLNDVLPNAEKIEQIKNIITSIGEKLKSLKEK | |
| peptide | LISCDFLHCENNDEIKTVKAIFNKLKDKGIYKAMGEFDIFINYVEKYIV | |
| | KT | |
| hIL-10R BP-129 | KKCDDVSFDYILKDLRSEFSKIKSFVQNNDKENMMLLSQSMLDKLTSCI | 304 |
| without signal | GCKSLSDMIKFYLNDVLPNAEKIEHIKNKITSIGEKLKSLKEKLISCDE | |
| peptide | LHCENHDEIKAVKTIFNKLKDKGIYKAMGEFDIFINHLEKYIVKK | |
| hIL-10R BP-130 | CVVASAKKCDDVSFDYILKDLRSEFIKIKSFVQNNDQENMMLLSQSMLD | 305 |
| without signal | KLTSRIGCKSLSDMIKFYLNDVLPNAEKIEQIKNIITSIGEKLKSLKEK | |
| peptide | LISCDFLHCENNDEIKTVKAIFNKLKDKGIYKAMGEFDIFINYVEKYIV | |
| | KT | |
| hIL-10R BP-131 | CTVASAKKCDDVSFDYILKDLRSEFSKIKSFVQNNDKENMMLLSQSMLD | 306 |
| without signal | KLTSCIGCKSLSDMIKFYLNDVLPNAEKIEHIKNKITSIGEKLKSLKEK | |
| peptide | LISCDFLHCENHDEIKAVKTIFNKLKDKGIYKAMGEFDIFINHLEKYIV | |
| | KK | |
| hIL-10R BP-132 | ATTTTTIKNTKPQCRPEDYATRLQDLRVTFHRVKPTLDDYSVWLDGTVV | 307 |
| without signal | KGCWGCSVMDWLLRRYLEIVFPAGDHVYPGLKTELHSMRSTLESIYKDM | |
| peptide | RQCPLLGCGDKAVISRLSQEAERKSDNGTRKGLSELDTLFSRLEEYLHS | |
| | RK | |
| hIL-10R BP-133 | CTVASAKKCDDVSFDYILKDLRSEFSKIKSFVQNNDKENMMLLSQSMLD | 308 |
| without signal | KLTRCIGCKSLSDMIKFYLNDVLPNAEKIEHIKNKITSIGEKLKSLKER | |
| peptide | LISCDFLHCENHDEIKAVKTIFNELKDKGIYKAMGEFDIFINHLEKYIV | |
| | KK | |
| hIL-10R BP-134 | TDQCDNFPQMLRDLRDAFSRVKTFFQTKDEVDNLLLKESLLEDFKGYLG | 309 |
| without signal | CQALSEMIQFYLEEVMPQAENQDPEAKDHVNSLGENLKTLRLRLRRCHR | |
| peptide | FLPCENKSKAVEQIKMPLTSCRKKEFTKP | |
| hIL-10R BP-135 | KGRDSKPSPACDPMHGALAGIFKELRTTYRSVRETLQTKDTVYYVSLFH | 310 |
| without signal | EQLLQEMLSPVGCRVTNELMQHYLDGVLPRAFHCGYDNTTLNALHELSS | |
| peptide | SLSTLYQHMLKCPALACTGQTPAWTQFLDTEHKLDPWKGTVKATAEMDL | |
| | LLNYLETELLQS | |
| hIL-10R BP-136 | ATTTIKNTKPQCRPEDYATRLQDLRVTFHRVKPTLQREDDYSVWLDGTV | 311 |
| without signal | VKGCWGCSVMDWLLRRYLEIVFPAGDHVYPGLKTELHSMRSTLESIYKD | |
| peptide | MRQCEAERKSDNGTRKGLSELDTLFSRLEEYLHSRK | |
| hIL-10R BP-137 | AHDHEHKVPPACDPVHGNLAGIFKELRTIYTSIREGLQKKDTVYYTSLF | 312 |
| without signal | NDRVLQEMLSPMGCRVTNEIMEHYLDGVLPRASHLDYDNSTLNGLHAFA | |
| peptide | SSMQALYQHMLKCPALACTGKTPAWMYFLEVEHKLNPWRGTAKAAAEAD | |
| | LLLNYLETFLLQF | |
| hIL-10R BP-138 | ASKPPVDCDPIHGTLSRIIKEVRTGYGSIKQALQSKDTVYYVSLFHENL | 313 |
| without signal | LNEMLSPVGCRVTNELMQHYLDGVLPRAFQCGYDNTTLDGLHSLVSSLD | |
| peptide | ALYKHMLKCPALACTGQTPAWTQFLETEHKLDPWKGTIKATAEMDLLVN | |
| | YLETFLAQS | |
| hIL-10R BP-139 | ATTTTTIKNTKPQCRPEDYATRLQDLRVTFHRVKPTLQREDDYSVWLDG | 314 |
| without signal | TVVKGCWGCSVMDWLLRRYLEIVFPAGDHVYPGLKTELHSMRSTLESIY | |
| peptide | KDMRQCPLLGCGDKSVISR | |
| hIL-10R BP-140 | ATTTIKNTKPQCRPEDYATRLQDLRVTFHRVKPTLQREDDYSVWLDGTV | 315 |
| without signal | VKGCWGCSVMDWLLRRYLEIVFPAGDHVYPGLKTELHSMRSTLESIYKD | |
| peptide | MRQCVSVSVAALSAQR | |
| hIL-10R BP-141 | IIDTCYDDQERERTKSNSISSVTPEMCKGLKQLVSTKLKDARQKEKSVR | 316 |
| without signal | DYFTSRDNDLDEMLLQGVKETHKKTCGCYVLYLLLSFYGKTIRDTIQSN | |
| peptide | KHKNLNTELTNLAVSVLSLEDLLEACGITCNPKKDSLLKRIEEYMKEHG | |
| | DDAIYKVIGEIEFLFQAIEKHVY | |
| hIL-10R BP-142 | NSIIDMCYDDQERERTKSNSISSITPDMCKGLKQLVATKLKDARQKEKL | 317 |
| without signal | VNSYFTSRDNDLTYMLLQGVRETHKKPCGCYVLYLLLTFYRKTIKDTIQ | |
| peptide | SKKHESINTELTNLAVTVLSLEDLLEACGITCNPKKDSLLKRIEGYTKE | |
| | HGDDAIYKVIGEIDELFQAIERHVY | |
| hIL-10R BP-143 | IIDTCYDDQERERTKSNSISSVTPEMCKGLKQLVATKLKDARQKEKLVN | 318 |
| without signal | DYFTGRDNDLSYMLLQGVRETHKKPCGCYVLYLLLSFYRKTIRDTIQSN | |
| peptide | KHASINAELTNLAVSVLSLEDLLDACGITCNPKKDSLLKRIEEYMKEHG | |
| | DDAIYKLIGEIEFLFQAIERHVYT | |
| hIL-10R BP-144 | IIDTYDEDEDEDSIKLSSIGSITPEMCKNLKQLVASKLKDIRQKEKSLR | 319 |
| without signal | DYFTNLDDELDYMLLQGVGENHKKKCGCYILHLLLKFYSKTIRNTIQSE | |
| peptide | KHKNVNLELTNIAVSMLALEDLLEKCGITCNPKKDPLLKRIEDYMKQHG | |
| | DDGVNKAIAELEFLFQMIEKQVYI | |
| hIL-10R BP-145 | AAQCRKGTITSRLKMLRTAFEKVREFYEDRDEEETALASTEHLHGPESC | 320 |
| without signal | SVIDELITHYTKCVIPAANEEEGADLLSLDTLQVALENVKGLLANCQEE | |
| peptide | FGCKPPFSMRDYKKQYRQLNKEKNAGMIKAMGELGMLFNGIEERVIGM | |
| hIL-10R BP-146 | MYVQHGSDYCTTTVHADIASAISGMRAEYDSGLGHYFKSLVPHPDNPYD | 321 |
| without signal | TDDYKYMINNTNSYNCHALQSTINALLGMYGYVDIDEPHQLAMMKLATH | |
| peptide | TMQAAMLLNKCAKQLGCYHIPFDVETLHEAHPDDVMASLDTALNLMSMV | |
| | TNEI | |
| hIL-10R BP-147 | AAQCRKGTITSRLKMLRTAFEKVREFYEDSDEEETALASTEHLHGPESC | 322 |
| without signal | SVIDELITHYTKCVIPAANEEEGADLLSLDTLQVALENVKGLLANCQEE | |
| peptide | FGCKPPFSMRDYKKQYRQLNKEKNAGMIKAMGELGMLFNGIEERVIGM | |
| hIL-10R BP-148 | ATIKCVGMSTLFNPELIQLRRLFGDGIKDFFQNKDEDLDNAFLNEDVQR | 323 |
| without signal | ELASDCGCDHLMDMLSLYVNDTIPKGMKTEDAPSGLGQMGQLMSSLYRK | |
| peptide | MDMCWSELGCSHNTRLTLQEYADKKGGWDNKALGESDILFDALELFFSK | |
| | IK | |
| hIL-10R BP-149 | MYVQHGSDYCTTTVRADIASAISGMRAEYNNGLGDYFKSLAPHPNNPYD | 324 |
| without signal | TDDYKYMINSTNSYNCHALQSTINALLGMYGYVDIDEPHQLAMMKLATH | |
| peptide | TMQTAMLLNKCAAQLGCYHIPFDVETLHEAHPNDVMASLDTALNLMSMV | |
| | TNEI | |
| hIL-10R BP-150 | MYARRSGDYCTTTVRADIASAISGMRAEYNSGLRDYFKSLVPHPDNPYD | 325 |
| without signal | TDDYKYMLNNTNSYNCHALQSTINALLGMYGYVDIDESHQLAMMKLATH | |
| peptide | TMQTAMMLNKCAAQLGCYHIPFDLETLHEAHPDDVMASLDTALNLMSMI | |
| | TNEI | |
| hIL-10R BP-151 | MYVQHGSDYCTTTVRADIASAISGMRAEYNNGLGDYFKSLAPHPNNPYD | 326 |
| without signal | TDDYKYMINSTNSYNCHALQSTINALLGMYGYVDIDEPHQLAMMKLATH | |
| peptide | TMQTAMLLNKCAAQLGCYHIPFDVETLHEAHPDDVMASLDTALNLMSMV | |
| | TNEI | |
| hIL-10R BP-152 | NVHSGTEDNPCTNSKTVLNTLLNQIKQEYINNLLPYYKALTPKPVDVED | 327 |
| without signal | DSYTYSIQSTDYNCYTIYETLNFLLGDVFPRATTDATVRLSLAKIATSS | |
| peptide | QQASMLMNLCKKELACGPAPFDMIKLYHDTKEYGADNIMGTLDTPFQYF | |
| | VIV | |
| hIL-10R BP-153 | MYVQHGRGDYCTTTVRADIASAISGMRAEYDSGLGHYFKSLVPHPDNPY | 328 |
| without signal | DTDDYKYMINNTNSYNCHALQSTINALLGMYGYVDIDEPHQLAMMKLAT | |
| peptide | HTMQTAMLLNKCAEQLGCYHIPFDVEILHEAHPDDVMASLDTALNLMSM | |
| | VTNEI | |
| hIL-10R BP-154 | APATTPKDSCVYLIGQTPQLLRQLRNAYQAIIGADGSGVDEDDMPIYPS | 329 |
| without signal | DVMNELASTSVACDAIKKVLTMNIGILPNVTAAYPDKKSEVDEIGDNLS | |
| peptide | RLHQNIVNCRDFLKCEDLPHWHQMAENYKEKPMQGFSEMDFVFQSVEKE | |
| | LVAKDVKNMKTKRKH | |
| hIL-10R BP-155 | DDDPCTNVKTQLNTLFNQIKTEYDTNLKTYYQSIAPSAFDPFNNTNYLY | 330 |
| without signal | SVQGNDYKCYTIFETLSFLMGDVYPRATTNESVRLSLAKVATSSTQGAM | |
| peptide | VMNLCRQQLGCGPPPFDAKTLYDDRAEYGADDIMATLDTALAKFKLVLE | |
| | SENVV | |
| hIL-10R BP-156 | DDDPCTNVKTQLNTLFNQIKTEYDTNLKTYYQSIAPSAFDPFNNTNYLY | 331 |
| without signal | SVQGNDYKCYTIFETLSFLMGDVYPRATTNESVRLSLAKVATSSTQGAM | |
| peptide | VMNLCREQLGCGPPPFDAKTLYDDRAEYGADDIMATLDTALAKFKLVLE | |
| | SENVV | |
| hIL-10R BP-157 | RRRGDYCTTTVRADIASAISGMRAEYNSGLGDYFKSLVPHPDNPYDTDD | 332 |
| without signal | YKHMIDNANSYNCHALQSTINALLGMYGYVDIDEPHQLAMMKLATHTMQ | |
| peptide | TAMLLNKCAAQLGCYHIPEDLETLREAPPADVMASLDTALNLMSMITNE | |
| | I | |
| hIL-10R BP-158 | ASLSTHYNNYDLTRIATIDKDVCKRVAQHINDDEVNMRKLYETQLKNYF | 333 |
| without signal | QQLVPNPTDVFKDDSYMYMINGTDYNCHIIYETMRELSGDVFPFATETE | |
| peptide | AELQYMWKMMLGVSQLSAYIGNCYQYFKCGPAPFDPQVLYHDRELFHAD | |
| | TVMAYLDTAFSHETL | |
| hIL-10R BP-159 | LNCGIEHNELNNIKNIFFKVRNVVQADDVDHNLRILTPALLNNITVSET | 334 |
| without signal | CFFIYDMFELYLNDVFVKYTNTALKLNILKSLSSVANNFLAIENKVKKR | |
| peptide | RVKKNTVNVLEIKKLLLIDNNCKKLFSEIDIFLTWVMAKI | |
| hIL-10R BP-160 | LNCGIEHNELNNIKNIFFKVRNVVQADDVDHNLRILTPALLNNITVSET | 335 |
| without signal | CFFIYDMFELYLNDVFVKYTNTALKLNILKSLSSVANNFLAIENKVKKR | |
| peptide | RVKKNNVNVLEIKKLLLIDNNCKKLFSEIDIFLTWVMAKI | |
| hIL-10R BP-161 | APATTPKDSCVYLIGQTPQLLRQLRNAYQAIIGADGSGVDEDDMPIYPS | 336 |
| without signal | DVMNELASTSVACDAIKKVLTMNIGILPNVTAAYPDKKSEVDEIGDNLS | |
| peptide | RLHQNIVNCVSRTQHLCYD | |
| hIL-10R BP-162 | DNKYDSESGDDCPTLPTSLPHMLHELRAAFSRVKTFFQMKDQLDNMLLD | 337 |
| without signal | GSLLEDFKGYLGCQALSEMIQFYLEEVMPQAENHSPDQDKNKVNSLGEK | |
| peptide | LKTLRVRLRRCHRFLPCENKSKAVEQVKSAFSKLQEKGVYKAMSEFDIE | |
| | INYIEAYMTTKMKN | |
| hIL-10R BP-163 | DNKYDSESGNDCPTLPTSLPHMLHELRAAFSRVKTFFQMKDQLDNMLLD | 338 |
| without signal | GSLLEDFKGYLGCQALSEMIQFYLEEVMPQAENHSTGQEKDKVNSLGEK | |
| peptide | LKTLRVRLRRCHRFLPCENKSKAVEQVKSAFSKLQEKGVYKAMSEFDIE | |
| | INYIEAYMTTKMKN | |
| hIL-10R BP-164 | DNKYDSESGNDCPTLPTSLPHMLHELRAAFSRVKTFFQMKDQLDNMLLD | 339 |
| without signal | GSLLEDFKGYLGCQALSEMIQFYLEEVMPQAENHSTDQEKDKVNSLGEK | |
| peptide | LKTLRVRLRRCHRELPCENKSKAVEQVKSAFSKLQEKGVYKAMSEFDIE | |
| | INYIEAYMTTKMKN | |
| hIL-10R BP-165 | DNKYDSESGDDCPTLPTSLPHMLHELRAAFSRVKTFFQMKDQLDNMLLD | 340 |
| without signal | GSLLEDFKGYLGCQALSEMIQFYLEEVMPQAENHSTGQEKDKVNSLGEK | |
| peptide | LKTLRVRLRRCHRFLPCENKSKAVEQVKSAFSKLQEKGVYKAMSEFDIE | |
| | INYIEAYMTTKMKN | |
| hIL-10R BP-166 | DNRYDGQDGNDCPTLPTSLPHMLHELRAAFSRVKTFFQMKDQLDNMLLD | 341 |
| without signal | GSLLEDFKGYLGCQALSEMIQFYLEEVMPQAENHSPDQDKNKVNSLGEK | |
| peptide | LKTLRVRLRRCHRFLPCENKSKAVEQVKSAFSKLQEKGVYKAMSEFDIF | |
| | INYIEAYMTTKMKN | |
| hIL-10R BP-167 | ATTAIKNTKPQCRPEDYATRLQDLRVTFHRVKPTLQREDDYSVWLDGTV | 342 |
| without signal | VKGCWGCSVMDWLLRRYLEIVFPAGDHVYPGLKTELHSMRSTLESIYKD | |
| peptide | MRQCPLLGCGDKSVISRLSQEAERKSDNGTRKGLSELDTLFSRLEEYLH | |
| | SRK | |
| hIL-10R BP-168 | ATTTTIKNTKPQCRPEDYATRLQDLRVTFDRVKPTLQREDDYSVWLDGT | 343 |
| without signal | VVKGCWGCSVMDWLLRRYLEIVFPAGDHVYPGLKTELHSMRSTLESIYK | |
| peptide | DMRQCPLLGCGDKSVISRLSQEAEKKSDNGTRKGLSELDTLFSRLEEYL | |
| | HSRK | |
| hIL-10R BP-169 | ATTTTTIKNTKPQCRPEDYATRLQDLRVTFYRVKPTLQREDDYSVWLDG | 344 |
| without signal | TVVKGCWGCSVMDWLLRRYLEIVFPAGDHVYPGLKTELHSMRSTLESIY | |
| peptide | KDMRQCPLLGCGDKSVISRLSQEAERKSDNGTRKGLSELDTLFSRLEEY | |
| | LHSRK | |
| hIL-10R BP-170 | ATTTTTIKNTKPQCRPEDYATRLQDLRVTFHRVKPTLDDYSVWLDGTVV | 345 |
| without signal | KGCWGCSVMDWLLRRYLEIVFPAGDHVYPGLKTELHSMRSTLESIYKDM | |
| peptide | RQCPLLGCGDKSVISRLSQEAERKSDNGTRKGLSELDTLFSRLEEYLHS | |
| | RK | |
| hIL-10R BP-171 | STKKCDDVSEDYILKDLRSEFSKIKSFVQDNDQENMMLLSQSMLDKLTS | 346 |
| without signal | RIGCKSLSDMIKFYLNDVLPNAEKIEHMKNKITSIGEKLKSLKEKLISC | |
| peptide | DFLHCENHDEIKTVKTIFNKLKDKGIYKAMGEFDIFINYLEKYIVKK | |
| hIL-10R BP-172 | ATTTIKNTKPQCRPEDYATRLQDLRVTFHRVKPTLQREDDYSVWLDGDH | 347 |
| without signal | VYPGLKTELHSMRSTLESIYKDMRQCPLLGCGDKSVISRLSQEAERKSD | |
| peptide | NGTRKGLSELDTLFSRLEEYLHSRK | |
| hIL-10R BP-173 | MIGTCYDEDEEIERLKSNSISSITPGMCRNLKHSVMIRLIDARQIEASI | 348 |
| without signal | RSYFTDGDNNLSEMLLQGIREISKKKCGCYILNLMLRFYIQTIKHTILS | |
| peptide | NKHKDMNLELTNLAVTILSLESLLEKCGVTCNPVKDPLLTRIEEYTRKH | |
| | GDNAIYKTIGELEFLEDAIEKFV | |
| hIL-10R BP-174 | QCRKGTITIRLKMLRTAFEKVREFYEDRDEEETALASTEHLHGPESCSV | 349 |
| without signal | IDELITHYTKCVIPAANEEEGADLRSLDTLQFALENVKGLLANCQEEFG | |
| peptide | CKPPFSMRDYKKQYRQLNKEKNAGMIKAMGELGMLFNGIEERVIGM | |
| hIL-10R BP-175 | QCRKGTITIRLKMLRTAFEKVREFYEDRDEEETALASTEHLHGPESCSV | 350 |
| without signal | IDELITHYTKCVIPAANEEEGADLRSLDTLQFALENVKGLLANCQEEFG | |
| peptide | CKPPFSMRDYKKQYRQLNKEKNAGMIKAMGELGMLFNGIEERVNGM | |
| hIL-10R BP-176 | QCRKGTITIRLKMLRTAFEKVREFYEDRDEEETALASTEHLHGPESCSV | 351 |
| without signal | IDELITHYTKCVIPAANEEEGADLLSLDTLQFALENVKGLLANCQEEFG | |
| peptide | CKPPFSMRDYKKQYRQLNKEKNAGMIKAMGELGMLFNGIEERVIGM | |
| hIL-10R BP-177 | AAQCRKGTITSRLKMLRTAFEKVREFYEDRDEEETALASTEHLHGPESC | 352 |
| without signal | SVIDELITHHTKCVIPAANEEEGADLLSLDTLQVALENVKGLLANCQEE | |
| peptide | FGCKPPFSMRDYKKQYRQLNKEKNAGMIKAMGELGMLFNGIEERVIGM | |
| hIL-10R BP-178 | ATTTIKNTKPQCRPEDYATRLQDLRVTFHRVKPTLVGHVGDHVYPGLKT | 353 |
| without signal | ELHSMRSTLESIYKDMRQCEAERKSDNGTRKGLSELDTLFSRLEEYLHS | |
| peptide | RK | |

In some embodiments, the amino acid sequence of the hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. In some embodiments, the amino acid sequence of the hIL-10R binding protein comprises an amino acid sequence at least 85% identical to the amino acid sequence of a polypeptide set forth in Table 2. In some embodiments, the amino acid sequence of the hIL-10R binding protein comprises an amino acid sequence at least 90% identical to the amino acid sequence of a polypeptide set forth in Table 2. In some embodiments, the amino acid sequence of the hIL-10R binding protein comprises an amino acid sequence at least 95% identical to the amino acid sequence of a polypeptide set forth in Table 2. In some embodiments, the amino acid sequence of the hIL-10R binding protein comprises an amino acid sequence at least 96% identical to the amino acid sequence of a polypeptide set forth in Table 2. In some embodiments, the amino acid sequence of the hIL-10R binding protein comprises an amino acid sequence at least 97% identical to the amino acid sequence of a polypeptide set forth in Table 2. In some embodiments, the amino acid sequence of the hIL-10R binding protein comprises an amino acid sequence at least 98% identical to the amino acid sequence of a polypeptide set forth in Table 2. In some embodiments, the amino acid sequence of the hIL-10R binding protein comprises an amino acid sequence at least 99% identical to the amino acid sequence of a polypeptide set forth in Table 2. In some embodiments, the amino acid sequence of the hIL-10R binding protein comprises an amino acid sequence at least 100% identical to the amino acid sequence of a polypeptide set forth in Table 2.

In some embodiments, the amino acid sequence of the hIL-10R binding protein comprises the amino acid sequence of a polypeptide set forth in Table 2, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence of a polypeptide set forth in Table 2, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence of a polypeptide set forth in Table 2, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence of a polypeptide set forth in Table 2, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence of a polypeptide set forth in Table 2, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. In some embodiments, the amino acid sequence of the hIL-10R binding protein consists of an amino acid sequence at least 85% identical to the amino acid sequence of a polypeptide set forth in Table 2. In some embodiments, the amino acid sequence of the hIL-10R binding protein consists of an amino acid sequence at least 90% identical to the amino acid sequence of a polypeptide set forth in Table 2. In some embodiments, the amino acid sequence of the hIL-10R binding protein consists of an amino acid sequence at least 95% identical to the amino acid sequence of a polypeptide set forth in Table 2. In some embodiments, the amino acid sequence of the hIL-10R binding protein consists of an amino acid sequence at least 96% identical to the amino acid sequence of a polypeptide set forth in Table 2. In some embodiments, the amino acid sequence of the hIL-10R binding protein consists of an amino acid sequence at least 97% identical to the amino acid sequence of a polypeptide set forth in Table 2. In some embodiments, the amino acid sequence of the hIL-10R binding protein consists of an amino acid sequence at least 98% identical to the amino acid sequence of a polypeptide set forth in Table 2. In some embodiments, the amino acid sequence of the hIL-10R binding protein consists of an amino acid sequence at least 99% identical to the amino acid sequence of a polypeptide set forth in Table 2. In some embodiments, the amino acid sequence of the hIL-10R binding protein consists of an amino acid sequence at least 100% identical to the amino acid sequence of a polypeptide set forth in Table 2.

In some embodiments, the amino acid sequence of the hIL-10R binding protein consists of the amino acid sequence of a polypeptide set forth in Table 2, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence of a polypeptide set forth in Table 2, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence of a polypeptide set forth in Table 2, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence of a polypeptide set forth in Table 2, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence of a polypeptide set forth in Table 2, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-353. In some embodiments, the amino acid sequence of hIL-10R binding protein comprises an amino acid sequence at least 85% identical to the amino acid sequence set forth in any one of SEQ ID NOS: 1-353.

In some embodiments, the amino acid sequence of hIL-10R binding protein comprises an amino acid sequence at least 90% identical to the amino acid sequence set forth in any one of SEQ ID NOS: 1-353. In some embodiments, the amino acid sequence of hIL-10R binding protein comprises an amino acid sequence at least 95% identical to the amino acid sequence set forth in any one of SEQ ID NOS: 1-353. In some embodiments, the amino acid sequence of hIL-10R binding protein comprises an amino acid sequence at least 96% identical to the amino acid sequence set forth in any one of SEQ ID NOS: 1-353. In some embodiments, the amino acid sequence of hIL-10R binding protein comprises an amino acid sequence at least 97% identical to the amino acid sequence set forth in any one of SEQ ID NOS: 1-353. In some embodiments, the amino acid sequence of hIL-10R binding protein comprises an amino acid sequence at least 98% identical to the amino acid sequence set forth in any one of SEQ ID NOS: 1-353. In some embodiments, the amino acid sequence of hIL-10R binding protein comprises an amino acid sequence at least 99% identical to the amino acid sequence set forth in any one of SEQ ID NOS: 1-353. In some embodiments, the amino acid sequence of hIL-10R binding protein comprises an amino acid sequence at least 100% identical to the amino acid sequence set forth in any one of SEQ ID NOS: 1-353.

In embodiments, the amino acid sequence of the hIL-10R binding protein comprises the amino acid sequence set forth in any one of SEQ ID NOS: 1-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-353. In some embodiments, the amino acid sequence of hIL-10R binding protein consists of an amino acid sequence at least 85% identical to the amino acid sequence set forth in any one of SEQ ID NOS: 1-353. In some embodiments, the amino acid sequence of hIL-10R binding protein consists of an amino acid sequence at least 90% identical to the amino acid sequence set forth in any one of SEQ ID NOS: 1-353. In some embodiments, the amino acid sequence of hIL-10R binding protein consists of an amino acid sequence at least 95% identical to the amino acid sequence set forth in any one of SEQ ID NOS: 1-353. In some embodiments, the amino acid sequence of hIL-10R binding protein consists of an amino acid sequence at least 96% identical to the amino acid sequence set forth in any one of SEQ ID NOS: 1-353. In some embodiments, the amino acid sequence of hIL-10R binding protein consists of an amino acid sequence at least 97% identical to the amino acid sequence set forth in any one of SEQ ID NOS: 1-353. In some embodiments, the amino acid sequence of hIL-10R binding protein consists of an amino acid sequence at least 98% identical to the amino acid sequence set forth in any one of SEQ ID NOS: 1-353. In some embodiments, the amino acid sequence of hIL-10R binding protein consists of an amino acid sequence at least 99% identical to the amino acid sequence set forth in any one of SEQ ID NOS: 1-353. In some embodiments, the amino acid sequence of hIL-10R binding protein consists of an amino acid sequence at least 100% identical to the amino acid sequence set forth in any one of SEQ ID NOS: 1-353.

In embodiments, the amino acid sequence of the hIL-10R binding protein consists of the amino acid sequence set forth in any one of SEQ ID NOS: 1-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-178. In some embodiments, the amino acid sequence of hIL-10R binding protein comprises an amino acid sequence at least 85% identical to the amino acid sequence set forth in any one of SEQ ID NOS: 1-178. In some embodiments, the amino acid sequence of hIL-10R binding protein comprises an amino acid sequence at least 90% identical to the amino acid sequence set forth in any one of SEQ ID NOS: 1-178. In some embodiments, the amino acid sequence of hIL-10R binding protein comprises an amino acid sequence at least 95% identical to the amino acid sequence set forth in any one of SEQ ID NOS: 1-178. In some embodiments, the amino acid sequence of hIL-10R binding protein comprises an amino acid sequence at least 96% identical to the amino acid sequence set forth in any one of SEQ ID NOS: 1-178. In some embodiments, the amino acid sequence of hIL-10R binding protein comprises an amino acid sequence at least 97% identical to the amino acid sequence set forth in any one of SEQ ID NOS: 1-178. In some embodiments, the amino acid sequence of hIL-10R binding protein comprises an amino acid sequence at least 98% identical to the amino acid sequence set forth in any one of SEQ ID NOS: 1-178. In some embodiments, the amino acid sequence of hIL-10R binding protein comprises an amino acid sequence at least 99% identical to the amino acid sequence set forth in any one of SEQ ID NOS: 1-178. In some embodiments, the amino acid sequence of hIL-10R binding protein comprises an amino acid sequence at least 100% identical to the amino acid sequence set forth in any one of SEQ ID NOS: 1-178.

In embodiments, the amino acid sequence of the hIL-10R binding protein comprises the amino acid sequence set forth in any one of SEQ ID NOS: 1-178, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-178, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-178, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-178, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-178, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-178. In some embodiments, the amino acid sequence of hIL-10R binding protein consists of an amino acid sequence at least 85% identical to the amino acid sequence set forth in any one of SEQ ID NOS: 1-178. In some embodiments, the amino acid sequence of hIL-10R binding protein consists of an amino acid sequence at least 90% identical to the amino acid sequence set forth in any one of SEQ ID NOS: 1-178. In some embodiments, the amino acid sequence of hIL-10R binding protein consists of an amino acid sequence at least 95% identical to the amino acid sequence set forth in any one of SEQ ID NOS: 1-178. In some embodiments, the amino acid sequence of hIL-10R binding protein consists of an amino acid sequence at least 96% identical to the amino acid sequence set forth in any one of SEQ ID NOS: 1-178. In some embodiments, the amino acid sequence of hIL-10R binding protein consists of an amino acid sequence at least 97% identical to the amino acid sequence set forth in any one of SEQ ID NOS: 1-178. In some embodiments, the amino acid sequence of hIL-10R binding protein consists of an amino acid sequence at least 98% identical to the amino acid sequence set forth in any one of SEQ ID NOS: 1-178. In some embodiments, the amino acid sequence of hIL-10R binding protein consists of an amino acid sequence at least 99% identical to the amino acid sequence set forth in any one of SEQ ID NOS: 1-178. In some embodiments, the amino acid sequence of hIL-10R binding protein consists of an amino acid sequence at least 100% identical to the amino acid sequence set forth in any one of SEQ ID NOS: 1-178.

In embodiments, the amino acid sequence of the hIL-10R binding protein consists of the amino acid sequence set forth in any one of SEQ ID NOS: 1-178, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-178, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-178, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-178, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-178, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 179-353. In some embodiments, the amino acid sequence of hIL-10R binding protein comprises an amino acid sequence at least 85% identical to the amino acid sequence set forth in any one of SEQ ID NOS: 179-353. In some embodiments, the amino acid sequence of hIL-10R binding protein comprises an amino acid sequence at least 90% identical to the amino acid sequence set forth in any one of SEQ ID NOS: 179-353. In some embodiments, the amino acid sequence of hIL-10R binding protein comprises an amino acid sequence at least 95% identical to the amino acid sequence set forth in any one of SEQ ID NOS: 179-353. In some embodiments, the amino acid sequence of hIL-10R binding protein comprises an amino acid sequence at least 96% identical to the amino acid sequence set forth in any one of SEQ ID NOS: 179-353. In some embodiments, the amino acid sequence of hIL-10R binding protein comprises an amino acid sequence at least 97% identical to the amino acid sequence set forth in any one of SEQ ID NOS: 179-353. In some embodiments, the amino acid sequence of hIL-10R binding protein comprises an amino acid sequence at least 98% identical to the amino acid sequence set forth in any one of SEQ ID NOS: 179-353. In some embodiments, the amino acid sequence of hIL-10R binding protein comprises an amino acid sequence at least 99% identical to the amino acid sequence set forth in any one of SEQ ID NOS: 179-353. In some embodiments, the amino acid sequence of hIL-10R binding protein comprises an amino acid sequence at least 100% identical to the amino acid sequence set forth in any one of SEQ ID NOS: 179-353.

In embodiments, the amino acid sequence of the hIL-10R binding protein comprises the amino acid sequence set forth in any one of SEQ ID NOS: 179-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 179-353. In some embodiments, the amino acid sequence of hIL-10R binding protein consists of an amino acid sequence at least 85% identical to the amino acid sequence set forth in any one of SEQ ID NOS: 179-353. In some embodiments, the amino acid sequence of hIL-10R binding protein consists of an amino acid sequence at least 90% identical to the amino acid sequence set forth in any one of SEQ ID NOS: 179-353. In some embodiments, the amino acid sequence of hIL-10R binding protein consists of an amino acid sequence at least 95% identical to the amino acid sequence set forth in any one of SEQ ID NOS: 179-353. In some embodiments, the amino acid sequence of hIL-10R binding protein consists of an amino acid sequence at least 96% identical to the amino acid sequence set forth in any one of SEQ ID NOS: 179-353. In some embodiments, the amino acid sequence of hIL-10R binding protein consists of an amino acid sequence at least 97% identical to the amino acid sequence set forth in any one of SEQ ID NOS: 179-353. In some embodiments, the amino acid sequence of hIL-10R binding protein consists of an amino acid sequence at least 98% identical to the amino acid sequence set forth in any one of SEQ ID NOS: 179-353. In some embodiments, the amino acid sequence of hIL-10R binding protein consists of an amino acid sequence at least 99% identical to the amino acid sequence set forth in any one of SEQ ID NOS: 179-353. In some embodiments, the amino acid sequence of hIL-10R binding protein consists of an amino acid sequence at least 100% identical to the amino acid sequence set forth in any one of SEQ ID NOS: 179-353.

In embodiments, the amino acid sequence of the hIL-10R binding protein consists of the amino acid sequence set forth in any one of SEQ ID NOS: 179-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-3. In some embodiments, the amino acid sequence of hIL-10R binding protein comprises an amino acid sequence at least 85% identical to the amino acid sequence set forth in any one of SEQ ID NOS: 1-3. In some embodiments, the amino acid sequence of hIL-10R binding protein comprises an amino acid sequence at least 90% identical to the amino acid sequence set forth in any one of SEQ ID NOS: 1-3. In some embodiments, the amino acid sequence of hIL-10R binding protein comprises an amino acid sequence at least 95% identical to the amino acid sequence set forth in any one of SEQ ID NOS: 1-3. In some embodiments, the amino acid sequence of hIL-10R binding protein comprises an amino acid sequence at least 96% identical to the amino acid sequence set forth in any one of SEQ ID NOS: 1-3. In some embodiments, the amino acid sequence of hIL-10R binding protein comprises an amino acid sequence at least 97% identical to the amino acid sequence set forth in any one of SEQ ID NOS: 1-3. In some embodiments, the amino acid sequence of hIL-10R binding protein comprises an amino acid sequence at least 98% identical to the amino acid sequence set forth in any one of SEQ ID NOS: 1-3. In some embodiments, the amino acid sequence of hIL-10R binding protein comprises an amino acid sequence at least 99% identical to the amino acid sequence set forth in any one of SEQ ID NOS: 1-3. In some embodiments, the amino acid sequence of hIL-10R binding protein comprises an amino acid sequence at least 100% identical to the amino acid sequence set forth in any one of SEQ ID NOS: 1-3.

In embodiments, the amino acid sequence of the hIL-10R binding protein comprises the amino acid sequence set forth in any one of SEQ ID NOS: 1-3, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-3, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-3, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-3, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 1-3, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-3. In some embodiments, the amino acid sequence of hIL-10R binding protein consists of an amino acid sequence at least 85% identical to the amino acid sequence set forth in any one of SEQ ID NOS: 1-3. In some embodiments, the amino acid sequence of hIL-10R binding protein consists of an amino acid sequence at least 90% identical to the amino acid sequence set forth in any one of SEQ ID NOS: 1-3. In some embodiments, the amino acid sequence of hIL-10R binding protein consists of an amino acid sequence at least 95% identical to the amino acid sequence set forth in any one of SEQ ID NOS: 1-3. In some embodiments, the amino acid sequence of hIL-10R binding protein consists of an amino acid sequence at least 96% identical to the amino acid sequence set forth in any one of SEQ ID NOS: 1-3. In some embodiments, the amino acid sequence of hIL-10R binding protein consists of an amino acid sequence at least 97% identical to the amino acid sequence set forth in any one of SEQ ID NOS: 1-3. In some embodiments, the amino acid sequence of hIL-10R binding protein consists of an amino acid sequence at least 98% identical to the amino acid sequence set forth in any one of SEQ ID NOS: 1-3. In some embodiments, the amino acid sequence of hIL-10R binding protein consists of an amino acid sequence at least 99% identical to the amino acid sequence set forth in any one of SEQ ID NOS: 1-3. In some embodiments, the amino acid sequence of hIL-10R binding protein consists of an amino acid sequence at least 100% identical to the amino acid sequence set forth in any one of SEQ ID NOS: 1-3.

In embodiments, the amino acid sequence of the hIL-10R binding protein consists of the amino acid sequence set forth in any one of SEQ ID NOS: 1-3, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-3, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-3, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-3, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 1-3, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1. In some embodiments, the amino acid sequence of hIL-10R binding protein comprises an amino acid sequence at least 85% identical to the amino acid sequence set forth in SEQ ID NO: 1. In some embodiments, the amino acid sequence of hIL-10R binding protein comprises an amino acid sequence at least 90% identical to the amino acid sequence set forth in SEQ ID NO: 1. In some embodiments, the amino acid sequence of hIL-10R binding protein comprises an amino acid sequence at least 95% identical to the amino acid sequence set forth in SEQ ID NO: 1. In some embodiments, the amino acid sequence of hIL-10R binding protein comprises an amino acid sequence at least 96% identical to the amino acid sequence set forth in SEQ ID NO: 1. In some embodiments, the amino acid sequence of hIL-10R binding protein comprises an amino acid sequence at least 97% identical to the amino acid sequence set forth in SEQ ID NO: 1. In some embodiments, the amino acid sequence of hIL-10R binding protein comprises an amino acid sequence at least 98% identical to the amino acid sequence set forth in SEQ ID NO: 1. In some embodiments, the amino acid sequence of hIL-10R binding protein comprises an amino acid sequence at least 99% identical to the amino acid sequence set forth in SEQ ID NO: 1. In some embodiments, the amino acid sequence of hIL-10R binding protein comprises an amino acid sequence at least 100% identical to the amino acid sequence set forth in SEQ ID NO: 1.

In embodiments, the amino acid sequence of the hIL-10R binding protein comprises the amino acid sequence set forth in SEQ ID NO: 1, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in SEQ ID NO: 1, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 1, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 1, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 1, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1. In some embodiments, the amino acid sequence of hIL-10R binding protein consists of an amino acid sequence at least 85% identical to the amino acid sequence set forth in SEQ ID NO: 1. In some embodiments, the amino acid sequence of hIL-10R binding protein consists of an amino acid sequence at least 90% identical to the amino acid sequence set forth in SEQ ID NO: 1. In some embodiments, the amino acid sequence of hIL-10R binding protein consists of an amino acid sequence at least 95% identical to the amino acid sequence set forth in SEQ ID NO: 1. In some embodiments, the amino acid sequence of hIL-10R binding protein consists of an amino acid sequence at least 96% identical to the amino acid sequence set forth in SEQ ID NO: 1. In some embodiments, the amino acid sequence of hIL-10R binding protein consists of an amino acid sequence at least 97% identical to the amino acid sequence set forth in SEQ ID NO: 1. In some embodiments, the amino acid sequence of hIL-10R binding protein consists of an amino acid sequence at least 98% identical to the amino acid sequence set forth in SEQ ID NO: 1. In some embodiments, the amino acid sequence of hIL-10R binding protein consists of an amino acid sequence at least 99% identical to the amino acid sequence set forth in SEQ ID NO: 1. In some embodiments, the amino acid sequence of hIL-10R binding protein consists of an amino acid sequence at least 100% identical to the amino acid sequence set forth in SEQ ID NO: 1.

In embodiments, the amino acid sequence of the hIL-10R binding protein consists of the amino acid sequence set forth in SEQ ID NO: 1, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in SEQ ID NO: 1, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 1, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 1, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 1, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 179-181. In some embodiments, the amino acid sequence of hIL-10R binding protein comprises an amino acid sequence at least 85% identical to the amino acid sequence set forth in any one of SEQ ID NOS: 179-181. In some embodiments, the amino acid sequence of hIL-10R binding protein comprises an amino acid sequence at least 90% identical to the amino acid sequence set forth in any one of SEQ ID NOS: 179-181. In some embodiments, the amino acid sequence of hIL-10R binding protein comprises an amino acid sequence at least 95% identical to the amino acid sequence set forth in any one of SEQ ID NOS: 179-181. In some embodiments, the amino acid sequence of hIL-10R binding protein comprises an amino acid sequence at least 96% identical to the amino acid sequence set forth in any one of SEQ ID NOS: 179-181. In some embodiments, the amino acid sequence of hIL-10R binding protein comprises an amino acid sequence at least 97% identical to the amino acid sequence set forth in any one of SEQ ID NOS: 179-181. In some embodiments, the amino acid sequence of hIL-10R binding protein comprises an amino acid sequence at least 98% identical to the amino acid sequence set forth in any one of SEQ ID NOS: 179-181. In some embodiments, the amino acid sequence of hIL-10R binding protein comprises an amino acid sequence at least 99% identical to the amino acid sequence set forth in any one of SEQ ID NOS: 179-181. In some embodiments, the amino acid sequence of hIL-10R binding protein comprises an amino acid sequence at least 100% identical to the amino acid sequence set forth in any one of SEQ ID NOS: 179-181.

In embodiments, the amino acid sequence of the hIL-10R binding protein comprises the amino acid sequence set forth in any one of SEQ ID NOS: 179-181, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-181, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-181, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-181, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 179-181, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 179-181. In some embodiments, the amino acid sequence of hIL-10R binding protein consists of an amino acid sequence at least 85% identical to the amino acid sequence set forth in any one of SEQ ID NOS: 179-181. In some embodiments, the amino acid sequence of hIL-10R binding protein consists of an amino acid sequence at least 90% identical to the amino acid sequence set forth in any one of SEQ ID NOS: 179-181. In some embodiments, the amino acid sequence of hIL-10R binding protein consists of an amino acid sequence at least 95% identical to the amino acid sequence set forth in any one of SEQ ID NOS: 179-181. In some embodiments, the amino acid sequence of hIL-10R binding protein consists of an amino acid sequence at least 96% identical to the amino acid sequence set forth in any one of SEQ ID NOS: 179-181. In some embodiments, the amino acid sequence of hIL-10R binding protein consists of an amino acid sequence at least 97% identical to the amino acid sequence set forth in any one of SEQ ID NOS: 179-181. In some embodiments, the amino acid sequence of hIL-10R binding protein consists of an amino acid sequence at least 98% identical to the amino acid sequence set forth in any one of SEQ ID NOS: 179-181. In some embodiments, the amino acid sequence of hIL-10R binding protein consists of an amino acid sequence at least 99% identical to the amino acid sequence set forth in any one of SEQ ID NOS: 179-181. In some embodiments, the amino acid sequence of hIL-10R binding protein consists of an amino acid sequence at least 100% identical to the amino acid sequence set forth in any one of SEQ ID NOS: 179-181.

In embodiments, the amino acid sequence of the hIL-10R binding protein consists of the amino acid sequence set forth in any one of SEQ ID NOS: 179-181, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-181, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-181, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-181, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 179-181, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 179. In some embodiments, the amino acid sequence of hIL-10R binding protein comprises an amino acid sequence at least 85% identical to the amino acid sequence set forth in SEQ ID NO: 179. In some embodiments, the amino acid sequence of hIL-10R binding protein comprises an amino acid sequence at least 90% identical to the amino acid sequence set forth in SEQ ID NO: 179. In some embodiments, the amino acid sequence of hIL-10R binding protein comprises an amino acid sequence at least 95% identical to the amino acid sequence set forth in SEQ ID NO: 179. In some embodiments, the amino acid sequence of hIL-10R binding protein comprises an amino acid sequence at least 96% identical to the amino acid sequence set forth in SEQ ID NO: 179. In some embodiments, the amino acid sequence of hIL-10R binding protein comprises an amino acid sequence at least 97% identical to the amino acid sequence set forth in SEQ ID NO: 179. In some embodiments, the amino acid sequence of hIL-10R binding protein comprises an amino acid sequence at least 98% identical to the amino acid sequence set forth in SEQ ID NO: 179. In some embodiments, the amino acid sequence of hIL-10R binding protein comprises an amino acid sequence at least 99% identical to the amino acid sequence set forth in SEQ ID NO: 179. In some embodiments, the amino acid sequence of hIL-10R binding protein comprises an amino acid sequence at least 100% identical to the amino acid sequence set forth in SEQ ID NO: 179.

In embodiments, the amino acid sequence of the hIL-10R binding protein comprises the amino acid sequence set forth in SEQ ID NO: 179, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in SEQ ID NO: 179, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 179, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 179, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 179, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 179. In some embodiments, the amino acid sequence of hIL-10R binding protein consists of an amino acid sequence at least 85% identical to the amino acid sequence set forth in SEQ ID NO: 179. In some embodiments, the amino acid sequence of hIL-10R binding protein consists of an amino acid sequence at least 90% identical to the amino acid sequence set forth in SEQ ID NO: 179. In some embodiments, the amino acid sequence of hIL-10R binding protein consists of an amino acid sequence at least 95% identical to the amino acid sequence set forth in SEQ ID NO: 179. In some embodiments, the amino acid sequence of hIL-10R binding protein consists of an amino acid sequence at least 96% identical to the amino acid sequence set forth in SEQ ID NO: 179. In some embodiments, the amino acid sequence of hIL-10R binding protein consists of an amino acid sequence at least 97% identical to the amino acid sequence set forth in SEQ ID NO: 179. In some embodiments, the amino acid sequence of hIL-10R binding protein consists of an amino acid sequence at least 98% identical to the amino acid sequence set forth in SEQ ID NO: 179. In some embodiments, the amino acid sequence of hIL-10R binding protein consists of an amino acid sequence at least 99% identical to the amino acid sequence set forth in SEQ ID NO: 179. In some embodiments, the amino acid sequence of hIL-10R binding protein consists of an amino acid sequence at least 100% identical to the amino acid sequence set forth in SEQ ID NO: 179.

In embodiments, the amino acid sequence of the hIL-10R binding protein consists of the amino acid sequence set forth in SEQ ID NO: 179, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in SEQ ID NO: 179, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 179, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 179, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 179, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 4-178. In some embodiments, the amino acid sequence of hIL-10R binding protein comprises an amino acid sequence at least 85% identical to the amino acid sequence set forth in any one of SEQ ID NOS: 4-178. In some embodiments, the amino acid sequence of hIL-10R binding protein comprises an amino acid sequence at least 90% identical to the amino acid sequence set forth in any one of SEQ ID NOS: 4-178. In some embodiments, the amino acid sequence of hIL-10R binding protein comprises an amino acid sequence at least 95% identical to the amino acid sequence set forth in any one of SEQ ID NOS: 4-178. In some embodiments, the amino acid sequence of hIL-10R binding protein comprises an amino acid sequence at least 96% identical to the amino acid sequence set forth in any one of SEQ ID NOS: 4-178. In some embodiments, the amino acid sequence of hIL-10R binding protein comprises an amino acid sequence at least 97% identical to the amino acid sequence set forth in any one of SEQ ID NOS: 4-178. In some embodiments, the amino acid sequence of hIL-10R binding protein comprises an amino acid sequence at least 98% identical to the amino acid sequence set forth in any one of SEQ ID NOS: 4-178. In some embodiments, the amino acid sequence of hIL-10R binding protein comprises an amino acid sequence at least 99% identical to the amino acid sequence set forth in any one of SEQ ID NOS: 4-178. In some embodiments, the amino acid sequence of hIL-10R binding protein comprises an amino acid sequence at least 100% identical to the amino acid sequence set forth in any one of SEQ ID NOS: 4-178.

In embodiments, the amino acid sequence of the hIL-10R binding protein comprises the amino acid sequence set forth in any one of SEQ ID NOS: 4-178, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 4-178, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 4-178, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 4-178, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 4-178, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 4-178. In some embodiments, the amino acid sequence of hIL-10R binding protein consists of an amino acid sequence at least 85% identical to the amino acid sequence set forth in any one of SEQ ID NOS: 4-178. In some embodiments, the amino acid sequence of hIL-10R binding protein consists of an amino acid sequence at least 90% identical to the amino acid sequence set forth in any one of SEQ ID NOS: 4-178. In some embodiments, the amino acid sequence of hIL-10R binding protein consists of an amino acid sequence at least 95% identical to the amino acid sequence set forth in any one of SEQ ID NOS: 4-178. In some embodiments, the amino acid sequence of hIL-10R binding protein consists of an amino acid sequence at least 96% identical to the amino acid sequence set forth in any one of SEQ ID NOS: 4-178. In some embodiments, the amino acid sequence of hIL-10R binding protein consists of an amino acid sequence at least 97% identical to the amino acid sequence set forth in any one of SEQ ID NOS: 4-178. In some embodiments, the amino acid sequence of hIL-10R binding protein consists of an amino acid sequence at least 98% identical to the amino acid sequence set forth in any one of SEQ ID NOS: 4-178. In some embodiments, the amino acid sequence of hIL-10R binding protein consists of an amino acid sequence at least 99% identical to the amino acid sequence set forth in any one of SEQ ID NOS: 4-178. In some embodiments, the amino acid sequence of hIL-10R binding protein consists of an amino acid sequence at least 100% identical to the amino acid sequence set forth in any one of SEQ ID NOS: 4-178.

In embodiments, the amino acid sequence of the hIL-10R binding protein consists of the amino acid sequence set forth in any one of SEQ ID NOS: 4-178, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 4-178, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 4-178, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 4-178, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 4-178, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 182-353. In some embodiments, the amino acid sequence of hIL-10R binding protein comprises an amino acid sequence at least 85% identical to the amino acid sequence set forth in any one of SEQ ID NOS: 182-353. In some embodiments, the amino acid sequence of hIL-10R binding protein comprises an amino acid sequence at least 90% identical to the amino acid sequence set forth in any one of SEQ ID NOS: 182-353. In some embodiments, the amino acid sequence of hIL-10R binding protein comprises an amino acid sequence at least 95% identical to the amino acid sequence set forth in any one of SEQ ID NOS: 182-353. In some embodiments, the amino acid sequence of hIL-10R binding protein comprises an amino acid sequence at least 96% identical to the amino acid sequence set forth in any one of SEQ ID NOS: 182-353. In some embodiments, the amino acid sequence of hIL-10R binding protein comprises an amino acid sequence at least 97% identical to the amino acid sequence set forth in any one of SEQ ID NOS: 182-353. In some embodiments, the amino acid sequence of hIL-10R binding protein comprises an amino acid sequence at least 98% identical to the amino acid sequence set forth in any one of SEQ ID NOS: 182-353. In some embodiments, the amino acid sequence of hIL-10R binding protein comprises an amino acid sequence at least 99% identical to the amino acid sequence set forth in any one of SEQ ID NOS: 182-353. In some embodiments, the amino acid sequence of hIL-10R binding protein comprises an amino acid sequence at least 100% identical to the amino acid sequence set forth in any one of SEQ ID NOS: 182-353.

In embodiments, the amino acid sequence of the hIL-10R binding protein comprises the amino acid sequence set forth in any one of SEQ ID NOS: 182-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 182-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 182-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 182-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 182-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 182-353. In some embodiments, the amino acid sequence of hIL-10R binding protein consists of an amino acid sequence at least 85% identical to the amino acid sequence set forth in any one of SEQ ID NOS: 182-353. In some embodiments, the amino acid sequence of hIL-10R binding protein consists of an amino acid sequence at least 90% identical to the amino acid sequence set forth in any one of SEQ ID NOS: 182-353. In some embodiments, the amino acid sequence of hIL-10R binding protein consists of an amino acid sequence at least 95% identical to the amino acid sequence set forth in any one of SEQ ID NOS: 182-353. In some embodiments, the amino acid sequence of hIL-10R binding protein consists of an amino acid sequence at least 96% identical to the amino acid sequence set forth in any one of SEQ ID NOS: 182-353. In some embodiments, the amino acid sequence of hIL-10R binding protein consists of an amino acid sequence at least 97% identical to the amino acid sequence set forth in any one of SEQ ID NOS: 182-353. In some embodiments, the amino acid sequence of hIL-10R binding protein consists of an amino acid sequence at least 98% identical to the amino acid sequence set forth in any one of SEQ ID NOS: 182-353. In some embodiments, the amino acid sequence of hIL-10R binding protein consists of an amino acid sequence at least 99% identical to the amino acid sequence set forth in any one of SEQ ID NOS: 182-353. In some embodiments, the amino acid sequence of hIL-10R binding protein consists of an amino acid sequence at least 100% identical to the amino acid sequence set forth in any one of SEQ ID NOS: 182-353.

In embodiments, the amino acid sequence of the hIL-10R binding protein consists of the amino acid sequence set forth in any one of SEQ ID NOS: 182-353, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 182-353, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 182-353, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 182-353, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 182-353, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 10 or 188. In some embodiments, the amino acid sequence of hIL-10R binding protein comprises an amino acid sequence at least 85% identical to the amino acid sequence set forth in any one of SEQ ID NOS: 10 or 188. In some embodiments, the amino acid sequence of hIL-10R binding protein comprises an amino acid sequence at least 85% identical to the amino acid sequence set forth in any one of SEQ ID NOS: 10 or 188. In some embodiments, the amino acid sequence of hIL-10R binding protein comprises an amino acid sequence at least 95% identical to the amino acid sequence set forth in any one of SEQ ID NOS: 10 or 188. In some embodiments, the amino acid sequence of hIL-10R binding protein comprises an amino acid sequence at least 96% identical to the amino acid sequence set forth in any one of SEQ ID NOS: 10 or 188. In some embodiments, the amino acid sequence of hIL-10R binding protein comprises an amino acid sequence at least 97% identical to the amino acid sequence set forth in any one of SEQ ID NOS: 10 or 188. In some embodiments, the amino acid sequence of hIL-10R binding protein comprises an amino acid sequence at least 98% identical to the amino acid sequence set forth in any one of SEQ ID NOS: 10 or 188. In some embodiments, the amino acid sequence of hIL-10R binding protein comprises an amino acid sequence at least 99% identical to the amino acid sequence set forth in any one of SEQ ID NOS: 10 or 188. In some embodiments, the amino acid sequence of hIL-10R binding protein comprises an amino acid sequence at least 100% identical to the amino acid sequence set forth in any one of SEQ ID NOS: 10 or 188.

In embodiments, the amino acid sequence of the hIL-10R binding protein comprises the amino acid sequence set forth in any one of SEQ ID NOS: 10 or 188, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 10 or 188, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 10 or 188, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 10 or 188, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in any one of SEQ ID NOS: 10 or 188, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 10 or 188. In some embodiments, the amino acid sequence of hIL-10R binding protein consists of an amino acid sequence at least 85% identical to the amino acid sequence set forth in any one of SEQ ID NOS: 10 or 188. In some embodiments, the amino acid sequence of hIL-10R binding protein consists of an amino acid sequence at least 90% identical to the amino acid sequence set forth in any one of SEQ ID NOS: 10 or 188. In some embodiments, the amino acid sequence of hIL-10R binding protein consists of an amino acid sequence at least 95% identical to the amino acid sequence set forth in any one of SEQ ID NOS: 10 or 188. In some embodiments, the amino acid sequence of hIL-10R binding protein consists of an amino acid sequence at least 96% identical to the amino acid sequence set forth in any one of SEQ ID NOS: 10 or 188. In some embodiments, the amino acid sequence of hIL-10R binding protein consists of an amino acid sequence at least 97% identical to the amino acid sequence set forth in any one of SEQ ID NOS: 10 or 188. In some embodiments, the amino acid sequence of hIL-10R binding protein consists of an amino acid sequence at least 98% identical to the amino acid sequence set forth in any one of SEQ ID NOS: 10 or 188. In some embodiments, the amino acid sequence of hIL-10R binding protein consists of an amino acid sequence at least 99% identical to the amino acid sequence set forth in any one of SEQ ID NOS: 10 or 188. In some embodiments, the amino acid sequence of hIL-10R binding protein consists of an amino acid sequence at least 100% identical to the amino acid sequence set forth in any one of SEQ ID NOS: 10 or 188.

In embodiments, the amino acid sequence of the hIL-10R binding protein consists of the amino acid sequence set forth in any one of SEQ ID NOS: 10 or 188, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 10 or 188, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 10 or 188, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 10 or 188, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in any one of SEQ ID NOS: 10 or 188, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of hIL-10R binding protein comprises an amino acid sequence at least 85% identical to the amino acid sequence set forth in SEQ ID NO: 10. In some embodiments, the amino acid sequence of hIL-10R binding protein comprises an amino acid sequence at least 90% identical to the amino acid sequence set forth in SEQ ID NO: 10. In some embodiments, the amino acid sequence of hIL-10R binding protein comprises an amino acid sequence at least 95% identical to the amino acid sequence set forth in SEQ ID NO: 10. In some embodiments, the amino acid sequence of hIL-10R binding protein comprises an amino acid sequence at least 96% identical to the amino acid sequence set forth in SEQ ID NO: 10. In some embodiments, the amino acid sequence of hIL-10R binding protein comprises an amino acid sequence at least 97% identical to the amino acid sequence set forth in SEQ ID NO: 10. In some embodiments, the amino acid sequence of hIL-10R binding protein comprises an amino acid sequence at least 98% identical to the amino acid sequence set forth in SEQ ID NO: 10. In some embodiments, the amino acid sequence of hIL-10R binding protein comprises an amino acid sequence at least 99% identical to the amino acid sequence set forth in SEQ ID NO: 10. In some embodiments, the amino acid sequence of hIL-10R binding protein comprises an amino acid sequence at least 100% identical to the amino acid sequence set forth in SEQ ID NO: 10.

In embodiments, the amino acid sequence of the hIL-10R binding protein comprises the amino acid sequence set forth in SEQ ID NO: 10, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in SEQ ID NO: 10, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 10, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 10, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 10, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of hIL-10R binding protein consists of an amino acid sequence at least 85% identical to the amino acid sequence set forth in SEQ ID NO: 10. In some embodiments, the amino acid sequence of hIL-10R binding protein consists of an amino acid sequence at least 90% identical to the amino acid sequence set forth in SEQ ID NO: 10. In some embodiments, the amino acid sequence of hIL-10R binding protein consists of an amino acid sequence at least 95% identical to the amino acid sequence set forth in SEQ ID NO: 10. In some embodiments, the amino acid sequence of hIL-10R binding protein consists of an amino acid sequence at least 96% identical to the amino acid sequence set forth in SEQ ID NO: 10. In some embodiments, the amino acid sequence of hIL-10R binding protein consists of an amino acid sequence at least 97% identical to the amino acid sequence set forth in SEQ ID NO: 10. In some embodiments, the amino acid sequence of hIL-10R binding protein consists of an amino acid sequence at least 98% identical to the amino acid sequence set forth in SEQ ID NO: 10. In some embodiments, the amino acid sequence of hIL-10R binding protein consists of an amino acid sequence at least 99% identical to the amino acid sequence set forth in SEQ ID NO: 10. In some embodiments, the amino acid sequence of hIL-10R binding protein consists of an amino acid sequence at least 100% identical to the amino acid sequence set forth in SEQ ID NO: 10.

In embodiments, the amino acid sequence of the hIL-10R binding protein consists of the amino acid sequence set forth in SEQ ID NO: 10, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in SEQ ID NO: 10, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 10, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 10, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 10, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 188. In some embodiments, the amino acid sequence of hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of hIL-10R binding protein comprises an amino acid sequence at least 85% identical to the amino acid sequence set forth in SEQ ID NO: 188. In some embodiments, the amino acid sequence of hIL-10R binding protein comprises an amino acid sequence at least 90% identical to the amino acid sequence set forth in SEQ ID NO: 188. In some embodiments, the amino acid sequence of hIL-10R binding protein comprises an amino acid sequence at least 95% identical to the amino acid sequence set forth in SEQ ID NO: 188. In some embodiments, the amino acid sequence of hIL-10R binding protein comprises an amino acid sequence at least 96% identical to the amino acid sequence set forth in SEQ ID NO: 188. In some embodiments, the amino acid sequence of hIL-10R binding protein comprises an amino acid sequence at least 97% identical to the amino acid sequence set forth in SEQ ID NO: 188. In some embodiments, the amino acid sequence of hIL-10R binding protein comprises an amino acid sequence at least 98% identical to the amino acid sequence set forth in SEQ ID NO: 188. In some embodiments, the amino acid sequence of hIL-10R binding protein comprises an amino acid sequence at least 99% identical to the amino acid sequence set forth in SEQ ID NO: 188. In some embodiments, the amino acid sequence of hIL-10R binding protein comprises an amino acid sequence at least 100% identical to the amino acid sequence set forth in SEQ ID NO: 188.

In embodiments, the amino acid sequence of the hIL-10R binding protein comprises the amino acid sequence set forth in SEQ ID NO: 188, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein comprises the amino acid sequence set forth in SEQ ID NO: 188, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 188, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 188, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein comprises the amino acid sequence set forth in SEQ ID NO: 188, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 188. In some embodiments, the amino acid sequence of hIL-10R binding protein consists of an amino acid sequence at least 85% identical to the amino acid sequence set forth in SEQ ID NO: 188. In some embodiments, the amino acid sequence of hIL-10R binding protein consists of an amino acid sequence at least 90% identical to the amino acid sequence set forth in SEQ ID NO: 188. In some embodiments, the amino acid sequence of hIL-10R binding protein consists of an amino acid sequence at least 95% identical to the amino acid sequence set forth in SEQ ID NO: 188. In some embodiments, the amino acid sequence of hIL-10R binding protein consists of an amino acid sequence at least 96% identical to the amino acid sequence set forth in SEQ ID NO: 188. In some embodiments, the amino acid sequence of hIL-10R binding protein consists of an amino acid sequence at least 97% identical to the amino acid sequence set forth in SEQ ID NO: 188. In some embodiments, the amino acid sequence of hIL-10R binding protein consists of an amino acid sequence at least 98% identical to the amino acid sequence set forth in SEQ ID NO: 188. In some embodiments, the amino acid sequence of hIL-10R binding protein consists of an amino acid sequence at least 99% identical to the amino acid sequence set forth in SEQ ID NO: 188. In some embodiments, the amino acid sequence of hIL-10R binding protein consists of an amino acid sequence at least 100% identical to the amino acid sequence set forth in SEQ ID NO: 188.

In embodiments, the amino acid sequence of the hIL-10R binding protein consists of the amino acid sequence set forth in SEQ ID NO: 188, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding protein consists of the amino acid sequence set forth in SEQ ID NO: 188, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 188, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 188, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding protein consists of the amino acid sequence set forth in SEQ ID NO: 188, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises one or more amino acid substitution at a position corresponding to an amino acid residue X25, X14, X18, X24, X28, X74, X90, X92, X96, X100 or X104, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises one or more amino acid substitution at a position corresponding to an amino acid residue D25, H14, N18, R24, D28, E74, H90, N92, E96, T100, or R104, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises the following amino acid substitutions N18Y, N92Q, T100D, an R104W, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179 and comprises the following amino acid substitutions D25A and E96A, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises one or more of the following sets of amino acid substitutions (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises one or more of the following sets of amino acid substitutions (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A, amino acid numbering relative to SEQ ID NO: 179.

In some embodiments, the amino acid sequence of the hIL-10R binding protein is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 1 or SEQ ID NO: 179, and comprises one or more amino acid substitution at a position corresponding to an amino acid residue D25, H14, N18, N21, M22, R24, D28, R32, E74, H90, N92, S93, E96, T100 and R104, amino acid numbering relative to SEQ ID NO: 179.

### 5.3 Potency & Affinity of hIL-10R Binding Agents

In some embodiments, the hIL-10R binding agent (e.g., the hIL-10R binding protein) (or e.g., a hIL-10R binding fusion protein or conjugate described herein) increases the level of STAT3 in cells expressing the hIL-10R on the surface relative to the level of STAT3 in the absence of the hIL-10R binding agent (e.g., the hIL-10R binding protein) (or e.g., a hIL-10R binding fusion protein or conjugate described herein) or relative to the level of STAT3 level in the presence of a suitable control (e.g., a reference hIL-10 binding agent (e.g., a reference hIL-10R binding protein) (e.g., SEQ ID NO: 1 or 179) (or a reference fusion or conjugate (e.g., a reference hIL-10R binding fusion protein)). In some embodiments, the hIL-10R binding agent (e.g., the hIL-10R binding protein) (or e.g., a hIL-10R binding fusion protein or conjugate described herein) increases the level of phosphorylated STAT3 in cells expressing the hIL-10R on the surface relative in the absence of the hIL-10R binding agent (e.g., the hIL-10R binding protein) (or e.g., a hIL-10R binding fusion protein or conjugate described herein) or relative to the level of phosphorylated STAT3 the presence of a suitable control (e.g., a reference hIL-10 binding protein (e.g., SEQ ID NO: 1 or 179)) (or a reference fusion or conjugate (e.g., a reference hIL-10R binding fusion protein)).

In some embodiments, the hIL-10R binding agent (e.g., the hIL-10R binding protein) (or e.g., a hIL-10R binding fusion protein or conjugate described herein) increases the level of phosphorylated STAT3 in cells expressing the hIL-10R on the surface with an EC50 of less than about 500 pM, 400 pM, 300 pM, 200 pM, 100 pM, 50 pM, 40 pM, 30 pM, 20 pM, 10 pM, 9 pM, 8 pM, 7 pM, 6 pM, 5 pM, 4 pM, 3 pM, 2 pM, 1 pM, 0.9 pM, 0.8 pM, 0.7 pM, 0.6 pM, 0.5 pM, 0.4 pM, 0.3 pM, 0.2 pM, or 0.1 pM In some embodiments, the hIL-10R binding agent (e.g., the hIL-10R binding protein) (or e.g., a hIL-10R binding fusion protein or conjugate described herein) increases the level of phosphorylated STAT3 cells expressing the hIL-10R on the surface with an EC50 of from about 500 pM-0.1 pM, 400 pM-0.1 pM, 300 pM-0.1 pM, 200 pM-0.1 pM, 100 pM-0.1 pM, 50 pM-0.1 pM, 25 pM-0.1 pM, 10 pM-0.1 pM, 5 pM-0.1 pM, or 1 pM-0.1 pM, 500 pM-0.5, 400 pM-0.5, 300 pM-0.5, 200 pM-0.5, 100 pM-0.5, 50 pM-0.5, 25 pM-0.5, 10 pM-0.5, 5 pM-0.5, or 1 pM-0.5 pM. In some embodiments, the hIL-10R binding agent (e.g., the hIL-10R binding protein) (or e.g., a hIL-10R binding fusion protein or conjugate described herein) increases the level of phosphorylated STAT3 in cells expressing the hIL-10R on the surface with an EC50 of no greater than about 0.1 pM, 0.2 pM, 0.3 pM, 0.4 pM, 0.5 pM, 0.6 pM, 0.7 pm, 0.8 pM, 0.9 pM, 1.0 pM, 5 pM, 10 pM, 20 pM, 30 pM, 40 pM, 50 pM, 60 pM, 70 pM, 80 pM, 90 pM, 100 pM, 200 pM, 300 pM, 400 pM, or 500 pM.

In some embodiments, the hIL-10R binding agent (e.g., the hIL-10R binding protein) (or e.g., a hIL-10R binding fusion protein or conjugate described herein) increases the level of phosphorylated STAT3 in cells expressing the hIL-10R on the surface with an EC50 that is at least about 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a suitable control (e.g., a reference hIL-10 binding protein (e.g., SEQ ID NO: 1 or 179)) (or a reference fusion or conjugate (e.g., a reference hIL-10R binding fusion protein)). In some embodiments, the hIL-10R binding agent (e.g., the hIL-10R binding protein) (or e.g., a hIL-10R binding fusion protein or conjugate described herein) increases the level of phosphorylated STAT3 in cells expressing the hIL-10R on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a suitable control (e.g., a reference hIL-10 binding protein (e.g., SEQ ID NO: 1 or 179)) (or a reference fusion or conjugate (e.g., a reference hIL-10R binding fusion protein))

In some embodiments, the hIL-10R binding agent (e.g., the hIL-10R binding protein) (or e.g., a hIL-10R binding fusion protein or conjugate described herein) binds to a cell expressing the hIL-10R on the surface with an EC50 of less than about 500 pM, 400 pM, 300 pM, 200 pM, 100 pM, 50 pM, 40 pM, 30 pM, 20 pM, 10 pM, 9 pM, 8 pM, 7 pM, 6 pM, 5 pM, 4 pM, 3 pM, 2 pM, 1 pM, 0.9 pM, 0.8 pM, 0.7 pM, 0.6 pM, 0.5 pM, 0.4 pM, 0.3 pM, 0.2 pM, or 0.1 pM. In some embodiments, the hIL-10R binding agent (e.g., the hIL-10R binding protein) (or e.g., a hIL-10R binding fusion protein or conjugate described herein) binds to a cell expressing the hIL-10R on the surface with an EC50 of from about 500 pM-0.1 pM, 400 pM-0.1 pM, 300 pM-0.1 pM, 200 pM-0.1 pM, 100 pM-0.1 pM, 50 pM-0.1 pM, 25 pM-0.1 pM, 10 pM-0.1 pM, 5 pM-0.1 pM, or 1 pM-0.1 pM, 500 pM-0.5, 400 pM-0.5, 300 pM-0.5, 200 pM-0.5, 100 pM-0.5, 50 pM-0.5, 25 pM-0.5, 10 pM-0.5, 5 pM-0.5, or 1 pM-0.5 pM. In some embodiments, the hIL-10R binding agent (e.g., the hIL-10R binding protein) (or e.g., a hIL-10R binding fusion protein or conjugate described herein) binds to a cell expressing the hIL-10R on the surface with an EC50 of no greater than about 0.1 pM, 0.2 pM, 0.3 pM, 0.4 pM, 0.5 pM, 0.6 pM, 0.7 pm, 0.8 pM, 0.9 pM, 1.0 pM, 5 pM, 10 pM, 20 pM, 30 pM, 40 pM, 50 pM, 60 pM, 70 pM, 80 pM, 90 pM, 100 pM, 200 pM, 300 pM, 400 pM, or 500 pM.

In some embodiments, the hIL-10R binding agent (e.g., the hIL-10R binding protein) (or e.g., a hIL-10R binding fusion protein or conjugate described herein) binds to a cell expressing the hIL-10R on the surface with an EC50 that is at least about 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a suitable control (e.g., a reference hIL-10 binding protein (e.g., SEQ ID NO: 1 or 179)) (or a reference fusion or conjugate (e.g., a reference hIL-10R binding fusion protein)). In some embodiments, the hIL-10R binding agent (e.g., the hIL-10R binding protein) (or e.g., a hIL-10R binding fusion protein or conjugate described herein) binds to a cell expressing the hIL-10R on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a suitable control (e.g., a reference hIL-10 binding protein (e.g., SEQ ID NO: 1 or 179)) (or a reference fusion or conjugate (e.g., a reference hIL-10R binding fusion protein)).

In some embodiments, the hIL-10R binding agent (e.g., the hIL-10R binding protein) (or e.g., a hIL-10R binding fusion protein or conjugate described herein) binds to a cell expressing hIL-10Rα on the surface with an EC50 of less than about 500 pM, 400 pM, 300 pM, 200 pM, 100 pM, 50 pM, 40 pM, 30 pM, 20 pM, 10 pM, 9 pM, 8 pM, 7 pM, 6 pM, 5 pM, 4 pM, 3 pM, 2 pM, 1 pM, 0.9 pM, 0.8 pM, 0.7 pM, 0.6 pM, 0.5 pM, 0.4 pM, 0.3 pM, 0.2 pM, or 0.1 pM. In some embodiments, the hIL-10R binding agent (e.g., the hIL-10R binding protein) (or e.g., a hIL-10R binding fusion protein or conjugate described herein) binds to a cell expressing hIL-10Rα on the surface with an EC50 of from about 500 pM-0.1 pM, 400 pM-0.1 pM, 300 pM-0.1 pM, 200 pM-0.1 pM, 100 pM-0.1 pM, 50 pM-0.1 pM, 25 pM-0.1 pM, 10 pM-0.1 pM, 5 pM-0.1 pM, or 1 pM-0.1 pM, 500 pM-0.5, 400 pM-0.5, 300 pM-0.5, 200 pM-0.5, 100 pM-0.5, 50 pM-0.5, 25 pM-0.5, 10 pM-0.5, 5 pM-0.5, or 1 pM-0.5 pM. In some embodiments, the hIL-10R binding agent (e.g., the hIL-10R binding protein) (or e.g., a hIL-10R binding fusion protein or conjugate described herein) binds to a cell expressing hIL-10Rα on the surface with an EC50 of no greater than about 0.1 pM, 0.2 pM, 0.3 pM, 0.4 pM, 0.5 pM, 0.6 pM, 0.7 pm, 0.8 pM, 0.9 pM, 1.0 pM, 5 pM, 10 pM, 20 pM, 30 pM, 40 pM, 50 pM, 60 pM, 70 pM, 80 pM, 90 pM, 100 pM, 200 pM, 300 pM, 400 pM, or 500 pM.

In some embodiments, the hIL-10R binding agent (e.g., the hIL-10R binding protein) (or e.g., a hIL-10R binding fusion protein or conjugate described herein) binds to a cell expressing hIL-10Rα on the surface with an EC50 that is at least about 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a suitable control (e.g., a reference hIL-10 binding protein (e.g., SEQ ID NO: 1 or 179)) (or a reference fusion or conjugate (e.g., a reference hIL-10R binding fusion protein)). In some embodiments, the hIL-10R binding agent (e.g., the hIL-10R binding protein) (or e.g., a hIL-10R binding fusion protein or conjugate described herein) binds to a cell expressing hIL-10Rα on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a suitable control (e.g., a reference hIL-10 binding protein (e.g., SEQ ID NO: 1 or 179)) (or a reference fusion or conjugate (e.g., a reference hIL-10R binding fusion protein)).

Assays suitable to measure the EC50 of an hIL-10R binding agent (e.g., a hIL-10R binding protein described herein) (or e.g., a hIL-10R binding fusion protein or conjugate described herein) are standard and known to the person of ordinary skill in the art. For example, the EC50 can be determined by constructing a dose-response curve and examining the effect of different concentrations of the hIL-10R binding agent (e.g., a hIL-10R binding protein) (or e.g., a hIL-10R binding fusion protein or conjugate) in inducing activity in a particular functional assay (e.g., STAT3 signaling, STAT3 phosphorylation, STAT3 inducible SEAP expression). An exemplary method of determining the EC50 of an hIL-10R binding protein described herein (or e.g., a hIL-10R binding fusion protein or conjugate described herein) is the hIL-10 HEKBlue reporter cell line (InvivoGen #hkb-il10). The hIL-10 HEKBlue reporter cell line expresses the hIL-10R and hIL-10Rβ subunits, human STAT3, and a STAT3-inducible SEAP (secreted embryonic alkaline phosphatase) reporter. Thereby, binding of a protein to the hIL-10R triggers JAK1/STAT3 signaling and the subsequent production of SEAP, which can be quantified using standard methods known in the art. Additionally for example, the level of phosphorylated SAT3 can be assessed by contacting cells expressing the hIL-10R with one or more concentration of an hIL-10R binding protein described herein, lysing the cells, and assessing the level of phosphorylated STAT3, e.g., by Western blot, FRET-based assay or chemiluminescent assay (e.g., AlphaLISA-based assay). The cells in the cell-based assay may be cells, such as HEK293 cells, which recombinantly express the hIL-10R and/or human STAT3; or cells that naturally express hIL-10R and human STAT3.

In some embodiments, the hIL-10R binding agent (e.g., the hIL-10R binding protein) (or e.g., a hIL-10R binding fusion protein or conjugate described herein) binds to hIL-10R with higher affinity relative to that of a reference hIL-10R binding agent (e.g., a reference hIL-10R binding protein) (e.g., a reference hIL-10R binding protein comprising the amino acid sequence set forth in SEQ ID NO: 1 or 179) (or a reference fusion or conjugate described herein (e.g., a reference hIL-10R binding fusion protein (e.g., a reference hIL-10R binding fusion protein)). Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of one protein to another (e.g., binding of a protein described herein to hIL-10R) include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.4 Nucleic Acid Molecules Encoding hIL-10R Binding Proteins

As described above, in some aspects and embodiments described herein, a hIL-10R binding protein (or a functional fragment and/or functional variant thereof) or a nucleic acid molecule comprising a coding region encoding the hIL-10R binding protein (or the functional fragment and/or functional variant thereof), see, e.g., § 5.2) is utilized (e.g., in compositions described herein (see, e.g., §§ 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc.). In some embodiments, the nucleic acid molecule comprising a coding region encoding the hIL-10R binding protein (or the functional fragment and/or functional variant thereof), see, e.g., § 5.2) is utilized.

In some embodiments, the nucleic acid molecule is a DNA molecule. In some embodiments, the nucleic acid molecule is an RNA (e.g., mRNA or circular RNA) molecule. In some embodiments, the RNA molecule is a translatable RNA. In some embodiments, the nucleic acid molecule is an mRNA molecule. In some embodiments, the nucleic acid molecule is a circular molecule.

In some embodiments, the nucleic acid molecule is a linear coding nucleic acid construct. In some embodiments, the nucleic acid molecule is contained within a vector (e.g., a non-viral vector (e.g., a plasmid), viral vector). In some embodiments, the nucleic acid molecule is contained within a non-viral vector. In some embodiments, the nucleic acid molecule is contained within a plasmid. In some embodiments, the nucleic acid molecule is contained within a viral vector. A more detailed description of vectors (e.g., non-viral (e.g., plasmids) and viral) for both RNA and DNA nucleic acids is provided in § 5.14.

In some embodiments, the nucleic acid molecule is modified or varied (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.4.1 DNA Molecules

In some embodiments, the nucleic acid molecule is a DNA molecule.

The coding DNA may also comprise one or more heterologous nucleic acid elements to mediate expression of the coding region. These include, e.g., promoter(s), enhancer(s), polyadenylation signal(s) (e.g., a poly(A) sequence), synthetic introns, transcriptional termination signals, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the hIL-10R binding protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

### 5.4.2 RNA Molecules

In some embodiments, the nucleic acid molecule is an RNA molecule. In some embodiments, the RNA molecule is a translatable RNA. In some embodiments, the RNA molecule is an mRNA, a self-replicating RNA, a circular RNA, a viral RNA, or a replicon RNA.

In some embodiments, the RNA molecule a circular RNA. Exemplary circular RNAs are described in e.g., U.S. Ser. No. 11/458,156, US20220143062, US20230212629, US20230072532, U.S. Ser. No. 11/203,767, U.S. Ser. No. 11/352,641, US20210371494, U.S. Ser. No. 11/766,449, US20230226096, WO2021189059, US20190345503, US20220288176, U.S. Ser. No. 11/560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA molecule is a mRNA. The basic components of an mRNA molecule typically include at least one coding region (e.g., a coding region encoding at least one hIL-10R binding protein described herein), a 5′-untranslated region (UTR), a 3′-UTR, a 5′-cap, and a poly(A) tail.

In some embodiments, the RNA molecule (e.g., mRNA, circular RNA) comprises at least one heterologous UTR. The UTRs may harbor regulatory sequence elements that determine the RNA (e.g., mRNA, circular RNA) turnover, stability, localization, and/or expression of operably linked coding sequence(s). The heterologous UTRs may be derived from a naturally occurring genes or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′-cap structure. In some embodiments, the 3′-UTR comprises a polyadenylation signal. In some embodiments, the RNA (e.g., mRNA) comprises at least one coding region encoding the hIL-10R binding protein described herein and 5′-UTR and/or a 3′-UTR. In some embodiments, the RNA (e.g., mRNA) comprises at least one coding sequence encoding an hIL-10R binding protein described herein operably connected to at least one heterologous 5′-UTR and at least one 3′-UTR.

In some embodiments, the RNA molecule (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. In some embodiments, the RNA molecule (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA molecule (e.g., mRNA) comprises a 5′-cap structure. In some embodiments, the 5′-cap structure stabilizes the RNA molecule (e.g., mRNA), enhances expression of the encoded hIL-10R binding protein, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′-cap structure comprises m7G, cap0, cap1, cap2, a modified cap0, or a modified cap1 structure.

In some embodiments, the RNA molecule (e.g., mRNA) comprises nucleotide analogues/modifications, e.g., backbone modifications, sugar modifications, and/or base modifications. A backbone modification in the context of the present disclosure is a modification, in which phosphates of the backbone of the nucleotides of the RNA molecule (e.g., mRNA) are chemically modified. A sugar modification in the context of the present disclosure is a chemical modification of the sugar of the nucleotides of the RNA molecule (e.g., mRNA). A base modification in the context of the present disclosure is a chemical modification of the base moiety of the nucleotides of the RNA molecule (e.g., mRNA).

In some embodiments, the RNA molecule (e.g., mRNA) comprises at least one chemically modified nucleotide. Exemplary nucleotide analogues/chemical modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA molecule (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA molecule (e.g., mRNA) comprises one or more pseudouridine (ψ), N1-methylpseudouridine (m1ψ), 5-methylcytosine, and 5-methoxyuridine. In some embodiments, essentially all, e.g., essentially 100% of the uracil in the coding sequence of the RNA molecule (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the mRNA encoding a hIL-10R binding protein described herein comprises: (i) a 5′-cap structure; (ii) a 5′-UTR; (iii) N1-methyl-pseudouridine, cytosine, adenine, and guanine; (iv) a 3′-UTR; and (v) a poly(A) region.

RNA molecules (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.5 Immunogens

In some aspects and embodiments described herein, an immunogen (e.g., an immunogenic protein (or a functional (e.g., immunogenic) fragment and/or functional (e.g., immunogenic) variant thereof) (or a nucleic acid molecule comprising a coding region encoding the immunogenic protein (or the functional (e.g., immunogenic) fragment and/or functional (e.g., immunogenic) variant thereof)) is utilized (e.g., in compositions described herein (see, e.g., §§ 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc.).

In some embodiments, the immunogen is an immunogenic protein (or a functional fragment and/or functional variant thereof). In some embodiments, the immunogen is a nucleic acid molecule comprising a coding region encoding the immunogenic protein (or the functional fragment and/or functional variant thereof). In some embodiments, an immunogenic protein (or a functional fragment and/or functional variant thereof) is utilized. In some embodiments, a nucleic acid molecule comprising a coding region encoding an immunogenic protein (or a functional fragment and/or functional variant thereof) is utilized.

In some embodiments, the immunogen is a pathogen immunogen. In some embodiments, the immunogen is an infective agent immunogen. In some embodiments, the immunogen is a viral, bacterial, fungal, or protozoal immunogen (e.g., a parasitic protozoal immunogen). In some embodiments, the immunogen is a tumor associated immunogen.

In some embodiments, the immunogen is a viral immunogen. Exemplary viruses from which immunogens may be derived include, but are not limited to, coronaviruses (e.g., SARS-CoV-2, SARS-CoV, MERS-CoV (e.g., SARS-CoV)), influenza viruses (e.g., influenza A, influenza B), respiratory syncytial viruses (RSV), rhinoviruses, parvoviruses (e.g., parvovirus B19), parainfluenza viruses, adenoviruses, varicella zoster viruses, papillomaviruses, yellow fever viruses, rabies lyssaviruses, variola viruses (e.g., variola major virus, variola minor virus, small pox virus, monkey pox virus), hepatitis B viruses, varicella viruses, tick-borne encephalitis (TBE) viruses, Japanese encephalitis viruses, rotaviruses, mumps viruses, rubella viruses, measles viruses, polioviruses, dengue viruses, sapoviruses, noroviruses, enteroviruses, and astroviruses. In some embodiments, the virus is a respiratory virus. In some embodiments, the virus is a coronavirus (e.g., a SARS-CoV-2 virus, a SARS-CoV virus, a MERS-CoV virus), an influenza virus (e.g., influenza A, influenza B), a respiratory syncytial virus (RSV), a rhinovirus, a parvovirus (e.g., parvovirus B19), a parainfluenza virus, or an adenovirus. In some embodiments, the virus is a rotavirus, an adenovirus, a sapovirus, a norovirus, an enterovirus, or an astrovirus.

In some embodiments, the immunogen is a respiratory virus immunogen. In some embodiments, the immunogen is a coronavirus immunogen (e.g., a SARS-CoV-2 virus immunogen, a SARS-CoV virus immunogen, a MERS-CoV virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

In some embodiments, the coronavirus immunogen is a SARS-CoV-2 virus immunogen, a SARS-CoV virus immunogen, a MERS-CoV virus immunogen. In some embodiments, the immunogen is a SARS-CoV-2 spike protein (or an immunogenic fragment or immunogenic variant thereof). In some embodiments, the immunogen is an influenza A virus immunogen. In some embodiments, the immunogen is an influenza B virus immunogen. In some embodiments, the immunogen is an influenza hemagglutinin protein immunogen or an influenza neuraminidase protein immunogen. In some embodiments, the immunogen is an RSV F protein immunogen or an RSV G protein immunogen.

In some embodiments, the immunogen is a bacterial immunogen. Exemplary bacteria from which immunogens may be derived include, but are not limited to, Streptococcus (e.g., Streptococcus pneumoniae), Neisseria (e.g., Neisseria meningitidis) (e.g., serogroups A, B, C, W, and Y), Salmonella (e.g., Salmonella Typhi), Vibrio (e.g., Vibrio cholerae, Vibrio parahaemolyticus), Clostridium (e.g., Clostridium tetani, Clostridium botulinum, Clostridium difficile), Haemophilus (e.g., Haemophilus influenzae), Bacillus (e.g., Bacillus anthracis), Mycobacterium (e.g., Mycobacterium tuberculosis), Campylobacter (e.g., Campylobacter jejuni), Shigella, Listeria (e.g., Listeria monocytogenes), Escherichia (e.g., Escherichia coli), Giardia (e.g., Giardia lamblia), Helicobacter (e.g., Heliobacter pylori), Yersinia (e.g., Yersinia enterocolitica), Cryptosporidium (e.g., Cryptosoridium parvum), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

In some embodiments, the immunogen is a protozoal immunogen. Exemplary protozoans from which immunogens may be derived include, but are not limited to, Leishmania (e.g., Leishmania major), Toxoplasma (e.g., Toxoplasma gondii), Plasmodium (e.g., Plasmodium falciparum), Leishmania (e.g., Leishmania infantum), Eimeria, Theileria (e.g., Theileria parva, Theileria annulate), Babesia (e.g., Babesia bovis, Babesia bigemina), Tritrichomonas (e.g., Tritrichomonas foetus), Giardia (e.g., Giardia lamblia), Sarcocystis (e.g., Sarcocystis neurona), Neospora (e.g., Neospora caninum), Entamoeba (e.g., Entamoeba Dispar, Entamoeba histolytica).

In some embodiments, the immunogen is a fungal immunogen. Exemplary fungi from which immunogens may be derived include, but are not limited to, Candidisis, Aspergillusis, Paracoccidioidomycosis, Blastomycosis, Coccidiomycosis, Histoplasmosis, Cryptococcusis, and Pneumocystosis.

In some embodiments, the immunogen is derived from a mucosal (e.g., respiratory mucosa, oral mucosa, gastrointestinal mucosa, or urogenital mucosa) pathogen. In some embodiments, the mucosal pathogen is a virus, bacteria, protozoa, or fungus. In some embodiments, the mucosal pathogen is a respiratory pathogen, an oral pathogen, a gastrointestinal pathogen, or a urogenital pathogen.

Exemplary mucosal pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, rotaviruses, adenoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), Entamoeba (e.g., Entamoeba Dispar, Entamoeba histolytica), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus), and Candidiasis.

Exemplary respiratory pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, and adenoviruses.

Exemplary gastrointestinal pathogens include, but are not limited to, adenoviruses, sapoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), and Entamoeba (e.g., Entamoeba Dispar, Entamoeba histolytica).

Exemplary urogenital pathogens include, but are not limited to, Candidiasis, Escherichia (e.g., Escherichia coli), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

In some embodiments, the immunogen is a tumor associated immunogen. Exemplary tumor associated immunogens include, but are not limited to, CD19; membrane spanning 4-domains A1 (MS4A1; CD20); CD22 (SIGLEC2); CD27 (TNFRSF7); TNFRSF8 (CD30); CD33 (SIGLEC3); CD37; CD38; CD40 (TNFRSF5), CD44; CD47; CD48 (SLAMF2); CD52; CD70 (TNFSF7; CD27L); 5′-nucleotidase ecto (NT5E; CD73), ectonucleoside triphosphate diphosphohydrolase 1 (CD39), CD74; CD79B; CD80; CD86; interleukin 3 receptor subunit alpha (IL3RA), prominin 1 (PROM1; CD133); TNFRSF9 (CD137); syndecan 1 (SDC1; CD138); CD200 molecule (CD200); alpha fetoprotein (AFP), BAG cochaperone 6 (BAG6); MET proto-oncogene, receptor tyrosine kinase (MET); KIT proto-oncogene, receptor tyrosine kinase (KIT); C-type lectin domain family 12 member A (CLEC12A; CD371); C-type lectin domain containing 9A (CLEC9A; CD370); cadherin 3 (CDH3); carbonic anhydrase 6 (CA6); carbonic anhydrase 9 (CA9); carcinoembryonic antigen related cell adhesion molecule 3 (CEACAM3); carcinoembryonic antigen related cell adhesion molecule 5 (CEACAM5); carcinoembryonic antigen related cell adhesion molecule 6 (CEACAM6); chorionic somatomammotropin hormone 1 (CSH1); coagulation factor III, tissue factor (F3); collectin subfamily member 10 (COLEC10; CLL1); delta like canonical Notch ligand 3 (DLL3); ectonucleotide pyrophosphatase/phosphodiesterase 3 (ENPP3); ephrin A1 (EFNA1); epidermal growth factor receptor (EGFR; ERBB; HER1); EGFR variant III (EGFRvIII); EPH receptor A2 (EPHA2); epithelial cell adhesion molecule (EPCAM); erb-b2 receptor tyrosine kinase 2 (ERBB2; HER-2/neu); fibroblast activation protein alpha (FAP); fibroblast growth factor receptor 2 (FGFR2); fibroblast growth factor receptor 3 (FGFR3); folate hydrolase 1 (FOLH1); folate receptor 1 (FOLR1); GD2 ganglioside; glycoprotein NMB (GPNMB; osteoactivin); guanylate cyclase 2C (GUCY2C); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G); MHC class Ipolypeptide-related sequence A (MICA); MHC class I polypeptide-related sequence B (MICB); integrin subunit beta 7 (ITGB7); leukocyte immunoglobulin like receptor B1 (LILRB1; ILT2); leukocyte immunoglobulin like receptor B2 (LILRB2; ILT4); LY6/PLAUR domain containing 3 (LYPD3); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member C3 (MAGEC3); MAGE family member D1 (MAGED1); MAGE family member D2 (MAGED2); mesothelin (MSLN); mucin 1 (MUC1) and splice variants thereof (e.g., including MUC1/A, C, D, X, Y, Z and REP); mucin 16 (MUC16; CA125); natural killer cell cytotoxicity receptor 3 ligand 1 (NCR3LG1; B7-H6); necdin, MAGE family member (NDN); nectin cell adhesion molecule 2 (NECTIN2); nectin cell adhesion molecule 4 (NECTIN4); SLIT and NTRK like family member 6 (SLITRK6); promyelocytic leukemia (PML); protein tyrosine kinase 7 (inactive) (PTK7); Poliovirus receptor (PVR) cell adhesion molecule (PVR); SLAM family member 6 (SLAMF6); SLAM family member 7 (SLAMF7); sialic acid binding Ig like lectin 7 (SIGLEC7); sialic acid binding Ig like lectin 9 (SIGLEC9); sialic acid binding Ig like lectin 10 (SIGLEC10); signal regulatory protein alpha (SIRPA) solute carrier family 34 (sodium phosphate), member 2 (SLC34A2); solute carrier family 39 member 6 (SLC39A6); STEAP family member 1 (STEAP1); suppression of tumorigenicity 2 (ST2); TNF receptor superfamily member 4 (TNFRSF4; OX40); TNF superfamily member 9 (TNFSF9; 4-1BB-L, CD137L); TNFRSF10A (DR4, TRAILR1); TNFRSF10B (DR5, TRAILR2); TNFRSF13B (BAFF); TNFRSF17 (BCMA); TNFRSF18 (GITR); transferrin (TF); transforming growth factor beta 1 (TGFB1) and isoforms thereof; triggering receptor expressed on myeloid cells 1 (TREM1); triggering receptor expressed on myeloid cells 2 (TREM2); trophoblast glycoprotein (TPBG); trophinin (TRO); tumor associated calcium signal transducer 2 (TACSTD2); Fucosyl GM1; sialyl Lewis adhesion molecule (sLe); and Lewis Y antigen.

### 5.6 Nucleic Acid Molecules Encoding Immunogens

As described above, in some aspects and embodiments described herein, an immunogenic protein (or a functional fragment and/or functional variant thereof) (or a nucleic acid molecule encoding the immunogenic protein (or the functional fragment and/or functional variant thereof)) is utilized (e.g., in compositions described herein (see, e.g., § 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc.). In some embodiments, a nucleic acid molecule encoding an immunogenic protein (or the functional fragment and/or functional variant thereof), see, e.g., § 5.5) is utilized.

In some embodiments, the nucleic acid molecule is a DNA molecule. In some embodiments, the nucleic acid molecule is an RNA (e.g., mRNA or circular RNA) molecule. In some embodiments, the RNA molecule is a translatable RNA. In some embodiments, the nucleic acid molecule is an mRNA molecule. In some embodiments, the nucleic acid molecule is a circular molecule.

In some embodiments, the nucleic acid molecule is a linear coding nucleic acid construct. In some embodiments, the nucleic acid molecule is contained within a vector (e.g., a non-viral vector (e.g., a plasmid), viral vector). In some embodiments, the nucleic acid molecule is contained within a non-viral vector. In some embodiments, the nucleic acid molecule is contained within a plasmid. In some embodiments, the nucleic acid molecule is contained within a viral vector. A more detailed description of vectors (e.g., non-viral (e.g., plasmids) and viral) for both RNA and DNA nucleic acids is provided in § 5.14.

In some embodiments, the nucleic acid molecule is modified or varied (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.6.1 DNA Molecules

In some embodiments, the nucleic acid molecule is a DNA molecule.

The coding DNA may also comprise one or more heterologous nucleic acid elements to mediate expression of the coding region. These include, e.g., promoter(s), enhancer(s), polyadenylation signal(s) (e.g., a poly(A) sequence), synthetic introns, transcriptional termination signals, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the immunogenic protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

### 5.6.2 RNA Molecules

In some embodiments, the nucleic acid molecule is an RNA molecule. In some embodiments, the RNA molecule is a translatable RNA. In some embodiments, the RNA molecule is an mRNA, a self-replicating RNA, a circular RNA, a viral RNA, or a replicon RNA.

In some embodiments, the RNA molecule a circular RNA. Exemplary circular RNAs are described in e.g., U.S. Ser. No. 11/458,156, US20220143062, US20230212629, US20230072532, U.S. Ser. No. 11/203,767, U.S. Ser. No. 11/352,641, US20210371494, U.S. Ser. No. 11/766,449, US20230226096, WO2021189059, US20190345503, US20220288176, U.S. Ser. No. 11/560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA molecule is a mRNA. The basic components of an mRNA molecule typically include at least one coding region (e.g., a coding region encoding at an immunogenic protein (e.g., described herein)), a 5′-untranslated region (UTR), a 3′-UTR, a 5′-cap, and a poly(A) tail.

In some embodiments, the RNA molecule (e.g., mRNA, circular RNA) comprises at least one heterologous UTR. The UTRs may harbor regulatory sequence elements that determine the RNA (e.g., mRNA, circular RNA) turnover, stability, localization, and/or expression of operably linked coding sequence(s). The heterologous UTRs may be derived from a naturally occurring genes or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′-cap structure. In some embodiments, the 3′-UTR comprises a polyadenylation signal. In some embodiments, the RNA (e.g., mRNA) comprises at least one coding region encoding the immunogenic protein (e.g., described herein) and 5′-UTR and/or a 3′-UTR. In some embodiments, the RNA (e.g., mRNA) comprises at least one coding sequence encoding an immunogenic protein (e.g., described herein) operably connected to at least one heterologous 5′-UTR and at least one 3′-UTR.

In some embodiments, the RNA molecule (e.g., mRNA) comprises apoly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. In some embodiments, the RNA molecule (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA molecule (e.g., mRNA) comprises a 5′-cap structure. In some embodiments, the 5′-cap structure stabilizes the RNA molecule (e.g., mRNA), enhances expression of the encoded immunogenic protein, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′-cap structure comprises m7G, cap0, cap1, cap2, a modified cap0, or a modified cap1 structure.

In some embodiments, the RNA molecule (e.g., mRNA) comprises nucleotide analogues/modifications, e.g., backbone modifications, sugar modifications, and/or base modifications. A backbone modification in the context of the present disclosure is a modification, in which phosphates of the backbone of the nucleotides of the RNA molecule (e.g., mRNA) are chemically modified. A sugar modification in the context of the present disclosure is a chemical modification of the sugar of the nucleotides of the RNA molecule (e.g., mRNA). A base modification in the context of the present disclosure is a chemical modification of the base moiety of the nucleotides of the RNA molecule (e.g., mRNA).

In some embodiments, the RNA molecule (e.g., mRNA) comprises at least one chemically modified nucleotide. Exemplary nucleotide analogues/chemical modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA molecule (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA molecule (e.g., mRNA) comprises one or more pseudouridine (ψ), N1-methylpseudouridine (m1ψ), 5-methylcytosine, and 5-methoxyuridine. In some embodiments, essentially all, e.g., essentially 100% of the uracil in the coding sequence of the RNA molecule (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the mRNA encoding a hIL-10R binding protein described herein comprises: (i) a 5′-cap structure; (ii) a 5′-UTR; (iii) N1-methyl-pseudouridine, cytosine, adenine, and guanine; (iv) a 3′-UTR; and (v) a poly(A) region.

RNA molecules (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.7 IgA Inducing Protein (IGIP)

In some aspects and embodiments described herein, an IGIP protein (e.g., human (hIGIP)) (or a functional fragment and/or functional variant thereof) or a nucleic acid molecule comprising a coding region encoding the IGIP (e.g., hIGIP) protein (or the functional fragment and/or functional variant thereof), see, e.g., § 5.4) is utilized (e.g., in compositions described herein (see, e.g., §§ 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc.). In some embodiments, an IGIP (e.g., hIGIP) protein (or a functional fragment and/or functional variant thereof) (e.g., described herein) is utilized. In some embodiments, a nucleic acid molecule comprising a coding region encoding the an IGIP (e.g., hIGIP) protein (or a functional fragment and/or functional variant thereof) (e.g., described herein) is utilized.

IGIP is a secreted protein produced by e.g., dendritic cells, that functions, inter alia, in the induction of IgA expression. See, e.g., Endsley M A, Njongmeta L M, Shell E, et al. Human IgA-inducing protein from dendritic cells induces IgA production by naive IgD+ B cells. J Immunol. 2009; 182(4):1854-1859. doi:10.4049/jimmunol.0801973; and WO2022056398A1, the entire contents of each of which are incorporated by reference herein for all purposes.

The amino acid sequence of a first reference immature hIGIP protein is set forth in SEQ ID NO: 570 and a second reference immature hIGIP protein is set forth in SEQ ID NO: 571. The amino acid sequence of a reference mature hIGIP protein is set forth in SEQ ID NOS: 572. See Table 13, herein.

TABLE 13
The Amino Acid Sequence of Reference hIGIP.

| | | SEQ ID |
|---|---|---|
| Description | Amino Acid Sequence | NO |
| hIGIP | MCSYYHMKKRSVSGCNITIFAVMESHLSAGKSPCGNQANVL | 570 |
| (Immature A-Signal | CISRLEFVQYQS | |
| Peptide Underlined) | | |
| hIGIP | MKKRSVSGCNITIFAVMESHLSAGKSPCGNQANVLCISRLE | 571 |
| (Immature B-Signal | FVQYQS | |
| Peptide Underlined) | | |
| hIGIP | KSPCGNQANVLCISRLEFVQYQS | 572 |
| (Mature-No Signal | | |
| Peptide) | | |

In some embodiments, the amino acid sequence of the IGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the IGIP protein comprises an amino acid sequence at least 85% identical to the amino acid sequence of a polypeptide set forth in Table 13. In some embodiments, the amino acid sequence of the IGIP protein comprises an amino acid sequence at least 90% identical to the amino acid sequence of a polypeptide set forth in Table 13. In some embodiments, the amino acid sequence of the IGIP protein comprises an amino acid sequence at least 95% identical to the amino acid sequence of a polypeptide set forth in Table 13. In some embodiments, the amino acid sequence of the IGIP protein comprises an amino acid sequence at least 100% identical to the amino acid sequence of a polypeptide set forth in Table 13.

In some embodiments, the amino acid sequence of the IGIP protein comprises the amino acid sequence of a protein set forth in Table 13, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein comprises the amino acid sequence of a protein set forth in Table 13, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of a protein set forth in Table 13, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of a protein set forth in Table 13, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the IGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the IGIP protein consists of an amino acid sequence at least 85% identical to the amino acid sequence of a polypeptide set forth in Table 13. In some embodiments, the amino acid sequence of the IGIP protein consists of an amino acid sequence at least 90% identical to the amino acid sequence of a polypeptide set forth in Table 13. In some embodiments, the amino acid sequence of the IGIP protein consists of an amino acid sequence at least 95% identical to the amino acid sequence of a polypeptide set forth in Table 13. In some embodiments, the amino acid sequence of the IGIP protein consists of an amino acid sequence at least 100% identical to the amino acid sequence of a polypeptide set forth in Table 13.

In some embodiments, the amino acid sequence of the IGIP protein consists of the amino acid sequence of a protein set forth in Table 13, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein consists of the amino acid sequence of a protein set forth in Table 13, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of a protein set forth in Table 13, and further consists of no more than about 1, 2, 3, 4, 5, 6, 7, 8, 9, or 10 amino acid variations (e.g., substitutions, additions, deletions, etc. (e.g., substitutions)).

In some embodiments, the IGIP protein (or the encoded protein) is a hIGIP protein. In some embodiments, the amino acid sequence of the hIGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13.

In some embodiments, the amino acid sequence of the hIGIP protein comprises an amino acid sequence at least 85% identical to the amino acid sequence of a polypeptide set forth in Table 13. In some embodiments, the amino acid sequence of the hIGIP protein comprises an amino acid sequence at least 90% identical to the amino acid sequence of a polypeptide set forth in Table 13. In some embodiments, the amino acid sequence of the hIGIP protein comprises an amino acid sequence at least 95% identical to the amino acid sequence of a polypeptide set forth in Table 13. In some embodiments, the amino acid sequence of the hIGIP protein comprises an amino acid sequence at least 100% identical to the amino acid sequence of a polypeptide set forth in Table 13.

In some embodiments, the amino acid sequence of the hIGIP protein comprises the amino acid sequence of a protein set forth in Table 13, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIGIP protein comprises the amino acid sequence of a protein set forth in Table 13, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence of a protein set forth in Table 13, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence of a protein set forth in Table 13, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIGIP protein consists of an amino acid sequence at least 85% identical to the amino acid sequence of a polypeptide set forth in Table 13. In some embodiments, the amino acid sequence of the hIGIP protein consists of an amino acid sequence at least 90% identical to the amino acid sequence of a polypeptide set forth in Table 13. In some embodiments, the amino acid sequence of the hIGIP protein consists of an amino acid sequence at least 95% identical to the amino acid sequence of a polypeptide set forth in Table 13. In some embodiments, the amino acid sequence of the hIGIP protein consists of an amino acid sequence at least 100% identical to the amino acid sequence of a polypeptide set forth in Table 13.

In some embodiments, the amino acid sequence of the hIGIP protein consists of the amino acid sequence of a protein set forth in Table 13, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the hIGIP protein consists of the amino acid sequence of a protein set forth in Table 13, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence of a protein set forth in Table 13, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence of a protein set forth in Table 13, and further consists of no more than about 1, 2, 3, 4, 5, 6, 7, 8, 9, or 10 amino acid variations (e.g., substitutions, additions, deletions, etc. (e.g., substitutions)).

In some embodiments, the amino acid sequence of IGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572. In some embodiments, the amino acid sequence of IGIP protein comprises an amino acid sequence at least 85% identical to the amino acid sequence set forth in any one of SEQ ID NOS: 570-572. In some embodiments, the amino acid sequence of IGIP protein comprises an amino acid sequence at least 90% identical to the amino acid sequence set forth in any one of SEQ ID NOS: 570-572. In some embodiments, the amino acid sequence of IGIP protein comprises an amino acid sequence at least 95% identical to the amino acid sequence set forth in any one of SEQ ID NOS: 570-572. In some embodiments, the amino acid sequence of IGIP protein comprises an amino acid sequence at least 100% identical to the amino acid sequence set forth in any one of SEQ ID NOS: 570-572.

In embodiments, the amino acid sequence of the IGIP protein comprises the amino acid sequence set forth in any one of SEQ ID NOS: 570-572, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein comprises the amino acid sequence set forth in any one of SEQ ID NOS: 570-572, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence set forth in any one of SEQ ID NOS: 570-572, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence set forth in any one of SEQ ID NOS: 570-572, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of IGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572. In some embodiments, the amino acid sequence of IGIP protein consists of an amino acid sequence at least 85% identical to the amino acid sequence set forth in any one of SEQ ID NOS: 570-572. In some embodiments, the amino acid sequence of IGIP protein consists of an amino acid sequence at least 90% identical to the amino acid sequence set forth in any one of SEQ ID NOS: 570-572. In some embodiments, the amino acid sequence of IGIP protein consists of an amino acid sequence at least 95% identical to the amino acid sequence set forth in any one of SEQ ID NOS: 570-572. In some embodiments, the amino acid sequence of IGIP protein consists of an amino acid sequence at least 100% identical to the amino acid sequence set forth in any one of SEQ ID NOS: 570-572.

In embodiments, the amino acid sequence of the IGIP protein consists of the amino acid sequence set forth in any one of SEQ ID NOS: 570-572, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein consists of the amino acid sequence set forth in any one of SEQ ID NOS: 570-572, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence set forth in any one of SEQ ID NOS: 570-572, and further consists of no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572. In some embodiments, the amino acid sequence of hIGIP protein comprises an amino acid sequence at least 85% identical to the amino acid sequence set forth in any one of SEQ ID NOS: 570-572. In some embodiments, the amino acid sequence of hIGIP protein comprises an amino acid sequence at least 90% identical to the amino acid sequence set forth in any one of SEQ ID NOS: 570-572. In some embodiments, the amino acid sequence of hIGIP protein comprises an amino acid sequence at least 95% identical to the amino acid sequence set forth in any one of SEQ ID NOS: 570-572. In some embodiments, the amino acid sequence of hIGIP protein comprises an amino acid sequence at least 100% identical to the amino acid sequence set forth in any one of SEQ ID NOS: 570-572.

In embodiments, the amino acid sequence of the hIGIP protein comprises the amino acid sequence set forth in any one of SEQ ID NOS: 570-572, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIGIP protein comprises the amino acid sequence set forth in any one of SEQ ID NOS: 570-572, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence set forth in any one of SEQ ID NOS: 570-572, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein comprises the amino acid sequence set forth in any one of SEQ ID NOS: 570-572, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572. In some embodiments, the amino acid sequence of hIGIP protein consists of an amino acid sequence at least 85% identical to the amino acid sequence set forth in any one of SEQ ID NOS: 570-572. In some embodiments, the amino acid sequence of hIGIP protein consists of an amino acid sequence at least 90% identical to the amino acid sequence set forth in any one of SEQ ID NOS: 570-572. In some embodiments, the amino acid sequence of hIGIP protein consists of an amino acid sequence at least 95% identical to the amino acid sequence set forth in any one of SEQ ID NOS: 570-572. In some embodiments, the amino acid sequence of hIGIP protein consists of an amino acid sequence at least 100% identical to the amino acid sequence set forth in any one of SEQ ID NOS: 570-572.

In embodiments, the amino acid sequence of the hIGIP protein consists of the amino acid sequence set forth in any one of SEQ ID NOS: 570-572, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the hIGIP protein consists of the amino acid sequence set forth in any one of SEQ ID NOS: 570-572, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence set forth in any one of SEQ ID NOS: 570-572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP protein consists of the amino acid sequence set forth in any one of SEQ ID NOS: 570-572, and further consists of no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 570. In some embodiments, the amino acid sequence of hIGIP comprises an amino acid sequence at least 85%, identical to the amino acid sequence set forth in SEQ ID NO: 570. In some embodiments, the amino acid sequence of hIGIP comprises an amino acid sequence at least 90% identical to the amino acid sequence set forth in SEQ ID NO: 570. In some embodiments, the amino acid sequence of hIGIP comprises an amino acid sequence at least 95% identical to the amino acid sequence set forth in SEQ ID NO: 570. In some embodiments, the amino acid sequence of hIGIP comprises an amino acid sequence at least 100% identical to the amino acid sequence set forth in SEQ ID NO: 570.

In embodiments, the amino acid sequence of the hIGIP comprises the amino acid sequence set forth in SEQ ID NO: 570, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIGIP comprises the amino acid sequence set forth in SEQ ID NO: 570, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 570, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 570, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 570, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 570. In some embodiments, the amino acid sequence of hIGIP consists of an amino acid sequence at least 85%, identical to the amino acid sequence set forth in SEQ ID NO: 570. In some embodiments, the amino acid sequence of hIGIP consists of an amino acid sequence at least 90% identical to the amino acid sequence set forth in SEQ ID NO: 570. In some embodiments, the amino acid sequence of hIGIP consists of an amino acid sequence at least 95% identical to the amino acid sequence set forth in SEQ ID NO: 570. In some embodiments, the amino acid sequence of hIGIP consists of an amino acid sequence at least 100% identical to the amino acid sequence set forth in SEQ ID NO: 570.

In embodiments, the amino acid sequence of the hIGIP consists of the amino acid sequence set forth in SEQ ID NO: 570, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the hIGIP consists of the amino acid sequence set forth in SEQ ID NO: 570, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 570, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 570, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 570, and further consists of no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 571. In some embodiments, the amino acid sequence of hIGIP comprises an amino acid sequence at least 85%, identical to the amino acid sequence set forth in SEQ ID NO: 571. In some embodiments, the amino acid sequence of hIGIP comprises an amino acid sequence at least 90% identical to the amino acid sequence set forth in SEQ ID NO: 571. In some embodiments, the amino acid sequence of hIGIP comprises an amino acid sequence at least 95% identical to the amino acid sequence set forth in SEQ ID NO: 571. In some embodiments, the amino acid sequence of hIGIP comprises an amino acid sequence at least 100% identical to the amino acid sequence set forth in SEQ ID NO: 571.

In embodiments, the amino acid sequence of the hIGIP comprises the amino acid sequence set forth in SEQ ID NO: 571, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIGIP comprises the amino acid sequence set forth in SEQ ID NO: 571, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 571, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 571, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 571, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 571. In some embodiments, the amino acid sequence of hIGIP consists of an amino acid sequence at least 85%, identical to the amino acid sequence set forth in SEQ ID NO: 571. In some embodiments, the amino acid sequence of hIGIP consists of an amino acid sequence at least 90% identical to the amino acid sequence set forth in SEQ ID NO: 571. In some embodiments, the amino acid sequence of hIGIP consists of an amino acid sequence at least 95% identical to the amino acid sequence set forth in SEQ ID NO: 571. In some embodiments, the amino acid sequence of hIGIP consists of an amino acid sequence at least 100% identical to the amino acid sequence set forth in SEQ ID NO: 571.

In embodiments, the amino acid sequence of the hIGIP consists of the amino acid sequence set forth in SEQ ID NO: 571, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the hIGIP consists of the amino acid sequence set forth in SEQ ID NO: 571, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 571, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 571, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 571, and further consists of no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 572. In some embodiments, the amino acid sequence of hIGIP comprises an amino acid sequence at least 85%, identical to the amino acid sequence set forth in SEQ ID NO: 572. In some embodiments, the amino acid sequence of hIGIP comprises an amino acid sequence at least 90% identical to the amino acid sequence set forth in SEQ ID NO: 572. In some embodiments, the amino acid sequence of hIGIP comprises an amino acid sequence at least 95% identical to the amino acid sequence set forth in SEQ ID NO: 572. In some embodiments, the amino acid sequence of hIGIP comprises an amino acid sequence at least 100% identical to the amino acid sequence set forth in SEQ ID NO: 572.

In embodiments, the amino acid sequence of the hIGIP comprises the amino acid sequence set forth in SEQ ID NO: 572, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIGIP comprises the amino acid sequence set forth in SEQ ID NO: 572, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 572, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP comprises the amino acid sequence set forth in SEQ ID NO: 572, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of hIGIP consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 572. In some embodiments, the amino acid sequence of hIGIP consists of an amino acid sequence at least 85%, identical to the amino acid sequence set forth in SEQ ID NO: 572. In some embodiments, the amino acid sequence of hIGIP consists of an amino acid sequence at least 90% identical to the amino acid sequence set forth in SEQ ID NO: 572. In some embodiments, the amino acid sequence of hIGIP consists of an amino acid sequence at least 95% identical to the amino acid sequence set forth in SEQ ID NO: 572. In some embodiments, the amino acid sequence of hIGIP consists of an amino acid sequence at least 100% identical to the amino acid sequence set forth in SEQ ID NO: 572.

In embodiments, the amino acid sequence of the hIGIP consists of the amino acid sequence set forth in SEQ ID NO: 572, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the hIGIP consists of the amino acid sequence set forth in SEQ ID NO: 572, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 572, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIGIP consists of the amino acid sequence set forth in SEQ ID NO: 572, and further consists of no more than about 1, 2, 3, 4, 5, 6, 7, 8, 9, or 10 amino acid variations (e.g., substitutions, additions, deletions, etc. (e.g., substitutions)).

In some embodiments, the IGIP protein is set forth in WO2022056398, the entire contents of which are incorporated herein by reference for all purposes.

In some embodiments, the amino acid sequence of the IGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in WO2022056398. In some embodiments, the amino acid sequence of the IGIP protein comprises an amino acid sequence at least 85% identical to the amino acid sequence of an IGIP protein set forth in WO2022056398. In some embodiments, the amino acid sequence of the IGIP protein comprises an amino acid sequence at least 90% identical to the amino acid sequence of an IGIP protein set forth in WO2022056398. In some embodiments, the amino acid sequence of the IGIP protein comprises an amino acid sequence at least 95% identical to the amino acid sequence of an IGIP protein set forth in WO2022056398. In some embodiments, the amino acid sequence of the IGIP protein comprises an amino acid sequence at least 100% identical to the amino acid sequence of an IGIP protein set forth in WO2022056398.

In some embodiments, the amino acid sequence of the IGIP protein comprises the amino acid sequence of an IGIP protein set forth in WO2022056398, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein comprises the amino acid sequence of an IGIP protein set forth in WO2022056398, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in WO2022056398, and further comprises or consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises or consists of the amino acid sequence of an IGIP protein set forth in WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in WO2022056398, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the IGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in WO2022056398. In some embodiments, the amino acid sequence of the IGIP protein consists of an amino acid sequence at least 85% identical to the amino acid sequence of an IGIP protein set forth in WO2022056398. In some embodiments, the amino acid sequence of the IGIP protein consists of an amino acid sequence at least 90% identical to the amino acid sequence of an IGIP protein set forth in WO2022056398. In some embodiments, the amino acid sequence of the IGIP protein consists of an amino acid sequence at least 95% identical to the amino acid sequence of an IGIP protein set forth in WO2022056398. In some embodiments, the amino acid sequence of the IGIP protein consists of an amino acid sequence at least 100% identical to the amino acid sequence of an IGIP protein set forth in WO2022056398.

In some embodiments, the amino acid sequence of the IGIP protein consists of the amino acid sequence of an IGIP protein set forth in WO2022056398, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein consists of the amino acid sequence of an IGIP protein set forth in WO2022056398, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of or consists of the amino acid sequence of an IGIP protein set forth in WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in WO2022056398, and further consists of no more than about 1, 2, 3, 4, 5, 6, 7, 8, 9, or 10 amino acid variations (e.g., substitutions, additions, deletions, etc. (e.g., substitutions)).

In some embodiments, the IGIP protein is set forth in Table 1 of WO2022056398, the entire contents of Table 1 of WO2022056398 are incorporated herein by reference for all purposes.

In some embodiments, the amino acid sequence of the IGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in Table 1 of WO2022056398. In some embodiments, the amino acid sequence of the IGIP protein comprises an amino acid sequence at least 85% identical to the amino acid sequence of an IGIP protein set forth in Table 1 of WO2022056398. In some embodiments, the amino acid sequence of the IGIP protein comprises an amino acid sequence at least 90% identical to the amino acid sequence of an IGIP protein set forth in Table 1 of WO2022056398. In some embodiments, the amino acid sequence of the IGIP protein comprises an amino acid sequence at least 95% identical to the amino acid sequence of an IGIP protein set forth in Table 1 of WO2022056398. In some embodiments, the amino acid sequence of the IGIP protein comprises an amino acid sequence at least 100% identical to the amino acid sequence of an IGIP protein set forth in Table 1 of WO2022056398.

In some embodiments, the amino acid sequence of the IGIP protein comprises the amino acid sequence of an IGIP protein set forth in Table 1 of WO2022056398, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein comprises the amino acid sequence of an IGIP protein set forth in Table 1 of WO2022056398, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in Table 1 of WO2022056398, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in Table 1 of WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in Table 1 of WO2022056398, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the IGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in Table 1 of WO2022056398. In some embodiments, the amino acid sequence of the IGIP protein consists of an amino acid sequence at least 85% identical to the amino acid sequence of an IGIP protein set forth in Table 1 of WO2022056398. In some embodiments, the amino acid sequence of the IGIP protein consists of an amino acid sequence at least 90% identical to the amino acid sequence of an IGIP protein set forth in Table 1 of WO2022056398. In some embodiments, the amino acid sequence of the IGIP protein consists of an amino acid sequence at least 95% identical to the amino acid sequence of an IGIP protein set forth in Table 1 of WO2022056398. In some embodiments, the amino acid sequence of the IGIP protein consists of an amino acid sequence at least 100% identical to the amino acid sequence of an IGIP protein set forth in Table 1 of WO2022056398.

In some embodiments, the amino acid sequence of the IGIP protein consists of the amino acid sequence of an IGIP protein set forth in Table 1 of WO2022056398, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein consists of the amino acid sequence of an IGIP protein set forth in Table 1 of WO2022056398, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in Table 1 of WO2022056398, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in Table 1 of WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in Table 1 of WO2022056398, and further consists of no more than about 1, 2, 3, 4, 5, 6, 7, 8, 9, or 10 amino acid variations (e.g., substitutions, additions, deletions, etc. (e.g., substitutions)).

In some embodiments, the IGIP protein is set forth in any one SEQ ID NOS: 1-12 of WO2022056398, SEQ ID NOS: 1-12 of WO2022056398 are incorporated herein by reference for all purposes.

In some embodiments, the amino acid sequence of the IGIP protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in any one SEQ ID NOS: 1-12 of WO2022056398. In some embodiments, the amino acid sequence of the IGIP protein comprises an amino acid sequence at least 85% identical to the amino acid sequence of an IGIP protein set forth in any one SEQ ID NOS: 1-12 of WO2022056398. In some embodiments, the amino acid sequence of the IGIP protein comprises an amino acid sequence at least 90% identical to the amino acid sequence of an IGIP protein set forth in any one SEQ ID NOS: 1-12 of WO2022056398. In some embodiments, the amino acid sequence of the IGIP protein comprises an amino acid sequence at least 95% identical to the amino acid sequence of an IGIP protein set forth in any one SEQ ID NOS: 1-12 of WO2022056398. In some embodiments, the amino acid sequence of the IGIP protein comprises an amino acid sequence at least 100% identical to the amino acid sequence of an IGIP protein set forth in any one SEQ ID NOS: 1-12 of WO2022056398.

In some embodiments, the amino acid sequence of the IGIP protein comprises the amino acid sequence of an IGIP protein set forth in any one SEQ ID NOS: 1-12 of WO2022056398, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein comprises the amino acid sequence of an IGIP protein set forth in any one SEQ ID NOS: 1-12 of WO2022056398, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in any one SEQ ID NOS: 1-12 of WO2022056398, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in any one SEQ ID NOS: 1-12 of WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein comprises the amino acid sequence of an IGIP protein set forth in any one SEQ ID NOS: 1-12 of WO2022056398, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the IGIP protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of an IGIP protein set forth in any one SEQ ID NOS: 1-12 of WO2022056398. In some embodiments, the amino acid sequence of the IGIP protein consists of an amino acid sequence at least 85% identical to the amino acid sequence of an IGIP protein set forth in any one SEQ ID NOS: 1-12 of WO2022056398. In some embodiments, the amino acid sequence of the IGIP protein consists of an amino acid sequence at least 90% identical to the amino acid sequence of an IGIP protein set forth in any one SEQ ID NOS: 1-12 of WO2022056398. In some embodiments, the amino acid sequence of the IGIP protein consists of an amino acid sequence at least 95% identical to the amino acid sequence of an IGIP protein set forth in any one SEQ ID NOS: 1-12 of WO2022056398. In some embodiments, the amino acid sequence of the IGIP protein consists of an amino acid sequence at least 100% identical to the amino acid sequence of an IGIP protein set forth in any one SEQ ID NOS: 1-12 of WO2022056398.

In some embodiments, the amino acid sequence of the IGIP protein consists of the amino acid sequence of an IGIP protein set forth in any one SEQ ID NOS: 1-12 of WO2022056398, and further consists of 1 or more but less than 15% (less than 12%, less than 10%, less than 8%), amino acid variations (e.g., substitutions, additions, deletions, etc. (e.g., substitutions)). In some embodiments, the amino acid sequence of the IGIP protein consists of the amino acid sequence of an IGIP protein set forth in any one SEQ ID NOS: 1-12 of WO2022056398, and further consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in any one SEQ ID NOS: 1-12 of WO2022056398, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in any one SEQ ID NOS: 1-12 of WO2022056398, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the IGIP protein consists of the amino acid sequence of an IGIP protein set forth in any one SEQ ID NOS: 1-12 of WO2022056398, and further consists of no more than about 1, 2, 3, 4, 5, 6, 7, 8, 9, or 10 amino acid variations (e.g., substitutions, additions, deletions, etc. (e.g., substitutions)).

### 5.8 Nucleic Acid Molecules Encoding IGIP Proteins

As described above, in some aspects and embodiments described herein, an IGIP (e.g., hIGIP) protein (or a functional fragment and/or functional variant thereof) (or a nucleic acid molecule comprising a coding region encoding the IGIP (e.g., hIGIP) protein (or the functional fragment and/or functional variant thereof)) is utilized (e.g., in compositions described herein (see, e.g., §§ 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc.). In some embodiments, a nucleic acid molecule comprising a coding region encoding the IGIP (e.g., hIGIP) protein (or the functional fragment and/or functional variant thereof), see, e.g., § 5.5) is utilized.

In some embodiments, the nucleic acid molecule is a DNA molecule. In some embodiments, the nucleic acid molecule is an RNA (e.g., mRNA or circular RNA) molecule. In some embodiments, the RNA molecule is a translatable RNA. In some embodiments, the nucleic acid molecule is an mRNA molecule. In some embodiments, the nucleic acid molecule is a circular molecule.

In some embodiments, the nucleic acid molecule is a linear coding nucleic acid construct. In some embodiments, the nucleic acid molecule is contained within a vector (e.g., a non-viral vector (e.g., a plasmid), viral vector). In some embodiments, the nucleic acid molecule is contained within a non-viral vector. In some embodiments, the nucleic acid molecule is contained within a plasmid. In some embodiments, the nucleic acid molecule is contained within a viral vector. A more detailed description of vectors (e.g., non-viral (e.g., plasmids) and viral) for both RNA and DNA nucleic acids is provided in § 5.14.

In some embodiments, the nucleic acid molecule is modified or varied (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.8.1 DNA Molecules

In some embodiments, the nucleic acid molecule is a DNA molecule.

The coding DNA may also comprise one or more heterologous nucleic acid elements to mediate expression of the coding region. These include, e.g., promoter(s), enhancer(s), polyadenylation signal(s) (e.g., a poly(A) sequence), synthetic introns, transcriptional termination signals, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the IGIP protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

### 5.8.2 RNA Molecules

In some embodiments, the nucleic acid molecule is an RNA molecule. In some embodiments, the RNA molecule is a translatable RNA. In some embodiments, the RNA molecule is an mRNA, a self-replicating RNA, a circular RNA, a viral RNA, or a replicon RNA.

In some embodiments, the RNA molecule a circular RNA. Exemplary circular RNAs are described in e.g., U.S. Ser. No. 11/458,156, US20220143062, US20230212629, US20230072532, U.S. Ser. No. 11/203,767, U.S. Ser. No. 11/352,641, US20210371494, U.S. Ser. No. 11/766,449, US20230226096, WO2021189059, US20190345503, US20220288176, U.S. Ser. No. 11/560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA molecule is a mRNA. The basic components of an mRNA molecule typically include at least one coding region (e.g., a coding region encoding an IGIP protein described herein), a 5′-untranslated region (UTR), a 3′-UTR, a 5′-cap, and a poly(A) tail.

In some embodiments, the RNA molecule (e.g., mRNA, circular RNA) comprises at least one heterologous UTR. The UTRs may harbor regulatory sequence elements that determine the RNA (e.g., mRNA, circular RNA) turnover, stability, localization, and/or expression of operably linked coding sequence(s). The heterologous UTRs may be derived from a naturally occurring genes or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′-cap structure. In some embodiments, the 3′-UTR comprises a polyadenylation signal. In some embodiments, the RNA (e.g., mRNA) comprises at least one coding region encoding the IGIP protein (e.g., described herein) and 5′-UTR and/or a 3′-UTR. In some embodiments, the RNA (e.g., mRNA) comprises at least one coding sequence encoding an IGIP protein (e.g., described herein) operably connected to at least one heterologous 5′-UTR and at least one 3′-UTR.

In some embodiments, the RNA molecule (e.g., mRNA) comprises apoly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. In some embodiments, the RNA molecule (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA molecule (e.g., mRNA) comprises a 5′-cap structure. In some embodiments, the 5′-cap structure stabilizes the RNA molecule (e.g., mRNA), enhances expression of the encoded IGIP protein, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′-cap structure comprises m7G, cap0, cap1, cap2, a modified cap0, or a modified cap1 structure.

In some embodiments, the RNA molecule (e.g., mRNA) comprises nucleotide analogues/modifications, e.g., backbone modifications, sugar modifications, and/or base modifications. A backbone modification in the context of the present disclosure is a modification, in which phosphates of the backbone of the nucleotides of the RNA molecule (e.g., mRNA) are chemically modified. A sugar modification in the context of the present disclosure is a chemical modification of the sugar of the nucleotides of the RNA molecule (e.g., mRNA). A base modification in the context of the present disclosure is a chemical modification of the base moiety of the nucleotides of the RNA molecule (e.g., mRNA).

In some embodiments, the RNA molecule (e.g., mRNA) comprises at least one chemically modified nucleotide. Exemplary nucleotide analogues/chemical modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA molecule (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA molecule (e.g., mRNA) comprises one or more pseudouridine (ψ), N1-methylpseudouridine (m1ψ), 5-methylcytosine, and 5-methoxyuridine. In some embodiments, essentially all, e.g., essentially 100% of the uracil in the coding sequence of the RNA molecule (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the mRNA encoding a hIL-10R binding protein described herein comprises: (i) a 5′-cap structure; (ii) a 5′-UTR; (iii) N1-methyl-pseudouridine, cytosine, adenine, and guanine; (iv) a 3′-UTR; and (v) a poly(A) region.

RNA molecules (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.9 Signal Peptides

In some embodiments, a protein described herein, e.g., a hIL-10R binding protein (e.g., described herein), an immunogenic protein (e.g., described herein), and/or an IGIP protein (e.g., described herein) (or any fusion protein or conjugate (e.g., described herein) comprising one or more of the foregoing) comprises a homologous or heterologous signal peptide operably connected to the N-terminus or C-terminus of the protein (i.e., a hIL-10R binding protein (e.g., described herein), an immunogenic protein (e.g., described herein), an IGIP protein (e.g., described herein), and/or any fusion protein or conjugate comprising one or more of the foregoing (e.g., described herein)).

In some embodiments, the signal peptide is operably connected to the N-terminus of the protein (e.g., the hIL-10R binding protein (e.g., described herein), the immunogenic protein (e.g., described herein), the IGIP protein (e.g., described herein), and/or any fusion protein or conjugate (e.g., described herein) comprising one or more of the foregoing). Commonly used signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 3.

TABLE 3
The amino acid sequence of exemplary signal
peptides.

| | | SEQ |
|---|---|---|
| Description | Amino Acid Sequence | ID NO |
| hIL-10 | MHSSALLCCLVLLTGVRA | 358 |
| hIL-2 | MYRMQLLSCIALSLALVINS | 359 |
| hOSM | MGVLLTQRTLLSLVLALLFPSMASM | 360 |
| hCTRB1 | MASLWLLSCESLVGAAFG | 361 |
| hTRY2 | MNLLLILTEVAAAVA | 362 |
| hINS | MALWMRLLPLLALLALWGPDPAAA | 363 |

In some embodiments, the amino acid sequence of the signal peptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 3. In some embodiments, the amino acid sequence of the signal peptide comprises or consists of the amino acid sequence of any one of the signal peptides set forth in Table 3. In some embodiments, the amino acid sequence of the signal peptide comprises or consists of the amino acid sequence of any one of the signal peptides set forth in Table 3, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of the signal peptides set forth in Table 3, comprising 1, 2, or 3 amino acid variations (e.g., substitutions, deletions, additions). In some embodiments, the amino acid sequence of the signal peptide comprises or consists of the amino acid sequence of any one of the signal peptides set forth in Table 3, and further comprises 1 or more but less than 15% (less than 12%, less than 10%, less than 8%), amino acid substitutions. In some embodiments, the amino acid sequence of the signal peptide comprises or consists of the amino acid sequence of any one of the signal peptides set forth in Table 3, comprising 1, 2, or 3 amino acid substitutions.

In some embodiments, the amino acid sequence of the signal peptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 358-363. In some embodiments, the amino acid sequence of the signal peptide comprises or consists of the amino acid sequence set forth in any one of SEQ ID NOS: 358-363. In some embodiments, the amino acid sequence of the signal peptide comprises or consists of the amino acid sequence set forth in any one of SEQ ID NOS: 358-363, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence set forth in any one of SEQ ID NOS: 358-363, comprising 1, 2, or 3 amino acid variations (e.g., substitutions, deletions, additions).

### 5.10 Fusions & Conjugates

In some embodiments, an agent (e.g., protein) described herein, including, e.g., a hIL-10R binding agent (e.g., a hIL-10R binding protein (or a functional fragment and/or functional variant thereof) or a nucleic acid molecule encoding the hIL-10R binding protein (or the functional fragment and/or functional variant thereof)), an immunogen (e.g., the immunogenic protein (or a functional fragment and/or functional variant thereof) or a nucleic acid molecule encoding the immunogenic protein (or the functional fragment and/or functional variant thereof)), and/or an IGIP (e.g., hIGIP) protein (or a functional fragment and/or functional variant thereof) or a nucleic acid molecule encoding the immunogenic protein (or the functional fragment and/or functional variant thereof)) is operably connected to a heterologous moiety (e.g., a heterologous polypeptide (or a nucleic acid molecule encoding the heterologous polypeptide)) forming a fusion or conjugate.

As such, further provided herein are, inter alia, fusion proteins comprising a protein described herein (including, e.g., a hIL-10R binding agent (e.g., a hIL-10R binding protein (or a functional fragment and/or functional variant thereof), an immunogen (e.g., the immunogenic protein (or a functional fragment and/or functional variant thereof), or an IGIP (e.g., hIGIP) protein (or a functional fragment and/or functional variant thereof)) and one or more heterologous proteins (or a functional fragment, functional variant, or domain thereof) (and nucleic acid molecules encoding the same).

For example, further provided herein are, inter alia, fusion proteins comprising a hIL-10R binding agent (e.g., a hIL-10R binding protein (or a functional fragment and/or functional variant thereof) and one or more heterologous proteins (or a functional fragment, functional variant, or domain thereof) (and nucleic acid molecules encoding the same). Further provided herein are, inter alia, conjugates comprising a hIL-10R binding agent (e.g., a hIL-10R binding protein (or a functional fragment and/or functional variant thereof) (or a nucleic acid molecule encoding the same) and one or more heterologous moieties (and nucleic acid molecules encoding the same).

The fusion proteins and conjugates described herein can be utilized, e.g., in compositions described herein (see, e.g., §§ 5.12, 5.13, 5.20), in nucleic acid molecules described herein (see, e.g., § 5.11), in vaccines described herein (see, e.g., § 5.13), in pharmaceutical compositions described herein (see, e.g., § 5.20), in methods described herein (see, e.g., § 5.21), in kits described herein (see, e.g., § 5.22), etc. In some embodiments, a fusion protein (or a functional fragment and/or functional variant thereof) (e.g., described herein) or conjugate is utilized. In some embodiments, a nucleic acid molecule comprising a coding region encoding the fusion protein (or a functional fragment and/or functional variant thereof) (e.g., described herein) or the conjugate is utilized.

Heterologous moieties include, but are not limited to, proteins, peptides, small molecules, nucleic acid molecules (e.g., DNA, RNA), carbohydrates, lipids, and synthetic polymers (e.g., polymers of PEG). In some embodiments, the heterologous moiety is a detectable protein (e.g., a fluorescent protein).

In some embodiments, the heterologous moiety (e.g., a heterologous polypeptide) imparts an additional function to the protein. For example, a heterologous moiety (e.g., a heterologous polypeptide) may function to promote or improve secretion of the encoded protein (e.g., via secretory signal peptides); increase half-life of the protein in vivo; promote or improve anchoring of the encoded protein in the plasma membrane (e.g., via transmembrane elements); promote or improve formation of antigen complexes (e.g., via multimerization domains or antigen clustering elements); promote or improve virus-like particle formation (VLP forming sequence); improve half-life of the protein; impart or increase detectability of the protein (e.g., in vitro, in vivo).

In some embodiments, the heterologous moiety is a half-life extension moiety. Exemplary half-life extension moieties include, but are not limited to, an immunoglobulin (e.g., human Ig (hIg), murine Ig (mIg)), a fragment of an Ig (e.g., hIg, mIg), an Ig (e.g., hIg, mIg) constant region, a fragment of an Ig (e.g., hIg, mIg) constant region, an Ig (e.g., hIg, mIg) Fc region human transferrin, human serum albumin (HSA), an HSA binding protein or peptide, and polyethylene glycol (PEG) (and polymers thereof). In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, a mIg, a fragment of a mIg, one or more mIg heavy chain constant region, a fragment of a mIg constant region, a mIg Fc region, human transferrin, human serum albumin (HSA), and an HSA binding protein or peptide. The protein (e.g., described herein) fused or conjugated to a half-life extending moiety or a half-life extending moiety can be evaluated for their pharmacokinetic properties utilizing standard in vivo methods known in the art.

### 5.10.1 Ig Fusion Proteins

In some embodiments, the heterologous polypeptide comprises an antibody. An antibody fusion can act to further target a protein (e.g., an hIL-10R binding agent (e.g., an hIL-10R binding protein (or a functional fragment and/or functional variant thereof)), an IGIP protein (e.g., described herein) (or a functional fragment and/or functional variant thereof), an immunogen (e.g., described herein) (e.g., an immunogenic protein (or a functional fragment and/or functional variant thereof)), and/or a nucleic acid molecule encoding the any of the foregoing), e.g., to a specified cell type expressing a specific cell surface protein. Exemplary antibodies include, full-length antibodies, scFv, Fab, single domain antibodies (e.g., VHH), scFv-Fc, Fab-Fc, and single domain antibody-Fc (e.g., VHH-Fc).

In some embodiments, the heterologous polypeptide comprises one or more immunoglobulin (Ig) heavy chain constant region (e.g., a CH2 region, a CH3 region, a hinge region, an Fc region). In some embodiments, the Ig is an IgG. In some embodiments, the IgG is IgG1, IgG2, IgG3, or IgG4.

In some embodiments, the heterologous polypeptide comprises an IgG CH2 region and an IgG CH3 region. In some embodiments, the heterologous polypeptide comprises a partial IgG hinge region, IgG CH2 region, and IgG CH3 region. In some embodiments, the heterologous polypeptide comprises an IgG hinge region, IgG CH2 region, and IgG CH3 region. In some embodiments, the heterologous polypeptide comprises an IgG1 CH2 region and an IgG1 CH3 region. In some embodiments, the heterologous polypeptide comprises a partial IgG1 hinge region, IgG1 CH2 region, and IgG1 CH3 region. In some embodiments, the heterologous polypeptide comprises an IgG1 hinge region, IgG1 CH2 region, and IgG1 CH3 region. In some embodiments, the heterologous polypeptide comprises an IgG4 CH2 region and a IgG4 CH3 region. In some embodiments, the heterologous polypeptide comprises a partial IgG4 hinge region, IgG4 CH2 region, and IgG4 CH3 region. In some embodiments, the heterologous polypeptide comprises an IgG4 hinge region, IgG4 CH2 region, and IgG4 CH3 region.

In some embodiments, the heterologous polypeptide consists of an IgG CH2 region and an IgG CH3 region. In some embodiments, the heterologous polypeptide consists of a partial IgG hinge region, IgG CH2 region, and IgG CH3 region. In some embodiments, the heterologous polypeptide consists of an IgG hinge region, IgG CH2 region, and IgG CH3 region. In some embodiments, the heterologous polypeptide consists of an IgG1 CH2 region and a IgG1 CH3 region. In some embodiments, the heterologous polypeptide consists of a partial IgG1 hinge region, IgG1 CH2 region, and IgG1 CH3 region. In some embodiments, the heterologous polypeptide consists of an IgG1 hinge region, IgG1 CH2 region, and IgG1 CH3 region. In some embodiments, the heterologous polypeptide consists of an IgG4 CH2 region and an IgG4 CH3 region. In some embodiments, the heterologous polypeptide consists of a partial IgG4 hinge region, IgG4 CH2 region, and IgG4 CH3 region. In some embodiments, the heterologous polypeptide consists of an IgG4 hinge region, IgG4 CH2 region, and IgG4 CH3 region.

In some embodiments, the heterologous polypeptide comprises an Ig Fc region. In some embodiments, the Ig Fc region comprises at least a portion of a hinge region, a CH2 region, and a CH3 region. In some embodiments, the Ig Fc region comprises a hinge region, a CH2 region, and a CH3 region. In some embodiments, the Ig Fc region comprises at least a portion of an IgG hinge region, an IgG CH2 region, and an IgG CH3 region. In some embodiments, the Ig Fc region comprises an IgG hinge region, an IgG CH2 region, and an IgG CH3 region. In some embodiments, the Ig Fc region comprises at least a portion of an IgG1 hinge region, an IgG1 CH2 region, and an IgG1 CH3 region. In some embodiments, the Ig Fc region comprises an IgG1 hinge region, an IgG1 CH2 region, and an IgG1 CH3 region. In some embodiments, the Ig Fc region comprises at least a portion of an IgG4 hinge region, an IgG4 CH2 region, and an IgG4 CH3 region. In some embodiments, the Ig Fc region comprises an IgG4 hinge region, an IgG4 CH2 region, and an IgG4 CH3 region.

In some embodiments, the heterologous polypeptide consists of an Ig Fc region. In some embodiments, the Ig Fc region consists of at least a portion of a hinge region, a CH2 region, and a CH3 region. In some embodiments, the Ig Fc region consists of a hinge region, a CH2 region, and a CH3 region. In some embodiments, the Ig Fc region consists of at least a portion of an IgG hinge region, an IgG CH2 region, and an IgG CH3 region. In some embodiments, the Ig Fc region consists of an IgG hinge region, an IgG CH2 region, and an IgG CH3 region. In some embodiments, the Ig Fc region consists of at least a portion of an IgG1 hinge region, an IgG1 CH2 region, and an IgG1 CH3 region. In some embodiments, the Ig Fc region consists of an IgG1 hinge region, an IgG1 CH2 region, and a IgG1 CH3 region. In some embodiments, the Ig Fc region consists of at least a portion of an IgG4 hinge region, an IgG4 CH2 region, and an IgG4 CH3 region. In some embodiments, the Ig Fc region consists of an IgG4 hinge region, an IgG4 CH2 region, and an IgG4 CH3 region.

In some embodiments, the heterologous polypeptide comprises one or more hIg heavy chain constant region (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the heterologous polypeptide comprises an hIgG CH2 region and an hIgG CH3 region. In some embodiments, the heterologous polypeptide comprises a partial hIgG hinge region, hIgG CH2 region, and hIgG CH3 region. In some embodiments, the heterologous polypeptide comprises an hIgG hinge region, hIgG CH2 region, and hIgG CH3 region. In some embodiments, the heterologous polypeptide comprises an hIgG1 CH2 region and an hIgG1 CH3 region. In some embodiments, the heterologous polypeptide comprises a partial hIgG1 hinge region, hIgG1 CH2 region, and hIgG1 CH3 region. In some embodiments, the heterologous polypeptide comprises an hIgG1 hinge region, hIgG1 CH2 region, and hIgG1 CH3 region. In some embodiments, the heterologous polypeptide comprises an hIgG4 CH2 region and an hIgG4 CH3 region. In some embodiments, the heterologous polypeptide comprises a partial hIgG4 hinge region, hIgG4 CH2 region, and hIgG4 CH3 region. In some embodiments, the heterologous polypeptide comprises an hIgG4 hinge region, hIgG4 CH2 region, and hIgG4 CH3 region.

In some embodiments, the heterologous polypeptide consists of an hIgG CH2 region and an hIgG CH3 region. In some embodiments, the heterologous polypeptide consists of a partial hIgG hinge region, hIgG CH2 region, and hIgG CH3 region. In some embodiments, the heterologous polypeptide consists of an hIgG hinge region, hIgG CH2 region, and hIgG CH3 region. In some embodiments, the heterologous polypeptide consists of an hIgG1 CH2 region and an hIgG1 CH3 region. In some embodiments, the heterologous polypeptide consists of a partial hIgG1 hinge region, hIgG1 CH2 region, and hIgG1 CH3 region. In some embodiments, the heterologous polypeptide consists of an hIgG1 hinge region, hIgG1 CH2 region, and hIgG1 CH3 region. In some embodiments, the heterologous polypeptide consists of an hIgG4 CH2 region and an hIgG4 CH3 region. In some embodiments, the heterologous polypeptide consists of a partial hIgG4 hinge region, hIgG4 CH2 region, and hIgG4 CH3 region. In some embodiments, the heterologous polypeptide consists of an hIgG4 hinge region, hIgG4 CH2 region, and hIgG4 CH3 region.

In some embodiments, the heterologous polypeptide comprises an hIg Fc region. In some embodiments, the hIg Fc region comprises at least a portion of a hinge region, a CH2 region, and a CH3 region. In some embodiments, the hIg Fc region comprises a hinge region, a CH2 region, and a CH3 region. In some embodiments, the hIg Fc region comprises at least a portion of an hIgG hinge region, an hIgG CH2 region, and an hIgG CH3 region. In some embodiments, the hIg Fc region comprises an hIgG hinge region, an hIgG CH2 region, and an hIgG CH3 region. In some embodiments, the hIg Fc region comprises at least a portion of an hIgG1 hinge region, an hIgG1 CH2 region, and an hIgG1 CH3 region. In some embodiments, the hIg Fc region comprises an hIgG1 hinge region, an hIgG1 CH2 region, and an hIgG1 CH3 region. In some embodiments, the hIg Fc region comprises at least a portion of an hIgG4 hinge region, an hIgG4 CH2 region, and an hIgG4 CH3 region. In some embodiments, the Ig Fc region comprises an hIgG4 hinge region, an hIgG4 CH2 region, and an hIgG4 CH3 region.

In some embodiments, the heterologous polypeptide consists of an hIg Fc region. In some embodiments, the h g Fe region consists of at least a portion of a hinge region, a CH2 region, and a CH3 region. In some embodiments, the hIg Fe region consists of a hinge region, a CH2 region, and a CH3 region. In some embodiments, the hIg Fe region consists of at least a portion of an hIgG hinge region, an hIgG CH2 region, and an IgG CH3 region. In some embodiments, the hIg Fe region consists of an hIgG hinge region, an hIgG CH2 region, and an hIgG CH3 region. In some embodiments, the hIg Fe region consists of at least a portion of an hIgG1 hinge region, an hIgG1 CH2 region, and an hIgG1 CH3 region. In some embodiments, the hg Fe region consists of an hIgG1 hinge region, an hIgG1 CH2 region, and a hIgG1 CH3 region. In some embodiments, the hIg Fe region consists of at least a portion of an hIgG4 hinge region, an hIgG4 CH2 region, and an IgG4 CH3 region. In some embodiments, the hIg Fe region consists of an hIgG4 hinge region, an hIgG4 CH2 region, and an hIgG4 CH3 region.

The amino acid sequence of exemplary reference hIgG1 and hIgG4 heavy chain constant regions, which can be incorporated in one or more of the embodiments described herein (e.g., fusion proteins and polypeptide), is provided in Table 4.

TABLE 4
The Amino Acid Sequence of Exemplary hIg chain constant region components.

| | | SEQ ID |
|---|---|---|
| Description | Amino Acid Sequence | NO |
| hIgG1 Hinge Region | EPKSCDKTHTCP | 364 |
| hIgG1 CH2 Region | PCPAPELLGGPSVFLFPPKPKDTLMISRTPEVTCVVVDVSH | 365 |
| | EDPEVKFNWYVDGVEVHNAKTKPREEQYNSTYRVVSVLTVL | |
| | HQDWLNGKEYKCKVSNKALPAPIEKTISKAK | |
| hIgG1 CH3 Region | GQPREPQVYTLPPSRDELTKNQVSLTCLVKGFYPSDIAVEW | 366 |
| With C-terminal Lysine | ESNGQPENNYKTTPPVLDSDGSFFLYSKLTVDKSRWQQGNV | |
| | FSCSVMHEALHNHYTQKSLSLSPGK | |
| hIgG1 CH3 Region | GQPREPQVYTLPPSRDELTKNQVSLTCLVKGFYPSDIAVEW | 367 |
| Without C-terminal | ESNGQPENNYKTTPPVLDSDGSFFLYSKLTVDKSRWQQGNV | |
| Lysine | FSCSVMHEALHNHYTQKSLSLSPG | |
| hIgG1 CH2 Region + | PCPAPELLGGPSVFLFPPKPKDTLMISRTPEVTCVVVDVSH | 368 |
| CH3 Region | EDPEVKFNWYVDGVEVHNAKTKPREEQYNSTYRVVSVLTVL | |
| With C-terminal Lysine | HQDWLNGKEYKCKVSNKALPAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPGK | |
| hIgG1 CH2 Region + | PCPAPELLGGPSVFLFPPKPKDTLMISRTPEVTCVVVDVSH | 369 |
| CH3 Region | EDPEVKFNWYVDGVEVHNAKTKPREEQYNSTYRVVSVLTVL | |
| Without C-terminal | HQDWLNGKEYKCKVSNKALPAPIEKTISKAKGQPREPQVYT | |
| Lysine | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| hIgG1 Partial Hinge | TCPPCPAPELLGGPSVFLFPPKPKDTLMISRTPEVTCVVVD | 370 |
| Region + CH2 Region + | VSHEDPEVKFNWYVDGVEVHNAKTKPREEQYNSTYRVVSVL | |
| CH3 Region | TVLHQDWLNGKEYKCKVSNKALPAPIEKTISKAKGQPREPQ | |
| With C-terminal Lysine | VYTLPPSRDELTKNQVSLTCLVKGFYPSDIAVEWESNGQPE | |
| | NNYKTTPPVLDSDGSFFLYSKLTVDKSRWQQGNVFSCSVMH | |
| | EALHNHYTQKSLSLSPGK | |
| hIgG1 Partial Hinge | TCPPCPAPELLGGPSVFLFPPKPKDTLMISRTPEVTCVVVD | 371 |
| Region + CH2 Region + | VSHEDPEVKFNWYVDGVEVHNAKTKPREEQYNSTYRVVSVL | |
| CH3 Region | TVLHQDWLNGKEYKCKVSNKALPAPIEKTISKAKGQPREPQ | |
| Without C-terminal | VYTLPPSRDELTKNQVSLTCLVKGFYPSDIAVEWESNGQPE | |
| Lysine | NNYKTTPPVLDSDGSFFLYSKLTVDKSRWQQGNVFSCSVMH | |
| | EALHNHYTQKSLSLSPG | |
| hIgG1 Hinge Region + | EPKSCDKTHTCPPCPAPELLGGPSVFLFPPKPKDTLMISRT | 372 |
| CH2 Region + CH3 | PEVTCVVVDVSHEDPEVKFNWYVDGVEVHNAKTKPREEQYN | |
| Region | STYRVVSVLTVLHQDWLNGKEYKCKVSNKALPAPIEKTISK | |
| With C-terminal Lysine | AKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| hIgG1 Hinge Region + | EPKSCDKTHTCPPCPAPELLGGPSVFLFPPKPKDTLMISRT | 373 |
| CH2 Region + CH3 | PEVTCVVVDVSHEDPEVKFNWYVDGVEVHNAKTKPREEQYN | |
| Region | STYRVVSVLTVLHQDWLNGKEYKCKVSNKALPAPIEKTISK | |
| Without C-terminal | AKGQPREPQVYTLPPSRDELTKNQVSLTCLVKGFYPSDIAV | |
| Lysine | EWESNGQPENNYKTTPPVLDSDGSFFLYSKLTVDKSRWQQG | |
| | NVFSCSVMHEALHNHYTQKSLSLSPG | |
| hIgG4 Hinge Region | ESKYGPPCPSCP | 374 |
| hIgG4 Hinge Region | AESKYGPPCPSCP | 375 |
| Variant | | |
| hIgG4 CH2 Region | APEFLGGPSVFLFPPKPKDTLMISRTPEVTCVVVDVSQEDP | 376 |
| | EVQFNWYVDGVEVHNAKTKPREEQFNSTYRVVSVLTVLHQD | |
| | WLNGKEYKCKVSNKGLPSSIEKTISKAK | |
| hIgG4 CH3 Region | GQPREPQVYTLPPSQEEMTKNQVSLTCLVKGFYPSDIAVEW | 377 |
| With C-terminal Lysine | ESNGQPENNYKTTPPVLDSDGSFFLYSRLTVDKSRWQEGNV | |
| | FSCSVMHEALHNHYTQKSLSLSLGK | |
| hIgG4 CH3 Region | GQPREPQVYTLPPSQEEMTKNQVSLTCLVKGFYPSDIAVEW | 378 |
| Without C-terminal | ESNGQPENNYKTTPPVLDSDGSFFLYSRLTVDKSRWQEGNV | |
| Lysine | FSCSVMHEALHNHYTQKSLSLSLG | |
| hIgG4 CH2 Region + | APEFLGGPSVFLFPPKPKDTLMISRTPEVTCVVVDVSQEDP | 379 |
| CH3 Region | EVQFNWYVDGVEVHNAKTKPREEQFNSTYRVVSVLTVLHQD | |
| With C-terminal Lysine | WLNGKEYKCKVSNKGLPSSIEKTISKAKGQPREPQVYTLPP | |
| | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLGK | |
| hIgG4 CH2 Region + | APEFLGGPSVFLFPPKPKDTLMISRTPEVTCVVVDVSQEDP | 380 |
| CH3 Region | EVQFNWYVDGVEVHNAKTKPREEQFNSTYRVVSVLTVLHQD | |
| Without C-terminal | WLNGKEYKCKVSNKGLPSSIEKTISKAKGQPREPQVYTLPP | |
| Lysine | SQEEMTKNQVSLTCLVKGFYPSDIAVEWESNGQPENNYKTT | |
| | PPVLDSDGSFFLYSRLTVDKSRWQEGNVESCSVMHEALHNH | |
| | YTQKSLSLSLG | |
| hIgG4 Partial Hinge | PCPSCPAPEFLGGPSVFLFPPKPKDTLMISRTPEVTCVVVD | 381 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| With C-terminal Lysine | VYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGK | |
| hIgG4 Partial Hinge | PCPSCPAPEFLGGPSVFLFPPKPKDTLMISRTPEVTCVVVD | 382 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPE | |
| Lysine | NNYKTTPPVLDSDGSFFLYSRLTVDKSRWQEGNVESCSVMH | |
| | EALHNHYTQKSLSLSLG | |
| hIgG4 Hinge Region + | ESKYGPPCPSCPAPEFLGGPSVFLFPPKPKDTLMISRTPEV | 383 |
| CH2 Region + CH3 | TCVVVDVSQEDPEVQFNWYVDGVEVHNAKTKPREEQENSTY | |
| Region | RVVSVLTVLHQDWLNGKEYKCKVSNKGLPSSIEKTISKAKG | |
| With C-terminal Lysine | QPREPQVYTLPPSQEEMTKNQVSLTCLVKGFYPSDIAVEWE | |
| | SNGQPENNYKTTPPVLDSDGSFFLYSRLTVDKSRWQEGNVE | |
| | SCSVMHEALHNHYTQKSLSLSLGK | |
| hIgG4 Hinge Region + | ESKYGPPCPSCPAPEFLGGPSVFLFPPKPKDTLMISRTPEV | 384 |
| CH2 Region + CH3 | TCVVVDVSQEDPEVQFNWYVDGVEVHNAKTKPREEQFNSTY | |
| Region | RVVSVLTVLHQDWLNGKEYKCKVSNKGLPSSIEKTISKAKG | |
| Without C-terminal | QPREPQVYTLPPSQEEMTKNQVSLTCLVKGFYPSDIAVEWE | |
| Lysine | SNGQPENNYKTTPPVLDSDGSFFLYSRLTVDKSRWQEGNVE | |
| | SCSVMHEALHNHYTQKSLSLSLG | |
| hIgG4 Hinge Region + | AESKYGPPCPSCPAPEFLGGPSVFLFPPKPKDTLMISRTPE | 385 |
| CH2 Region + CH3 | VTCVVVDVSQEDPEVQFNWYVDGVEVHNAKTKPREEQENST | |
| Region | YRVVSVLTVLHQDWLNGKEYKCKVSNKGLPSSIEKTISKAK | |
| Variant | GQPREPQVYTLPPSQEEMTKNQVSLTCLVKGFYPSDIAVEW | |
| With C-terminal Lysine | ESNGQPENNYKTTPPVLDSDGSFFLYSRLTVDKSRWQEGNV | |
| | FSCSVMHEALHNHYTQKSLSLSLGK | |
| hIgG4 Hinge Region + | AESKYGPPCPSCPAPEFLGGPSVFLFPPKPKDTLMISRTPE | 386 |
| CH2 Region + CH3 | VTCVVVDVSQEDPEVQFNWYVDGVEVHNAKTKPREEQENST | |
| Region | YRVVSVLTVLHQDWLNGKEYKCKVSNKGLPSSIEKTISKAK | |
| Variant | GQPREPQVYTLPPSQEEMTKNQVSLTCLVKGFYPSDIAVEW | |
| Without C-terminal | ESNGQPENNYKTTPPVLDSDGSFFLYSRLTVDKSRWQEGNV | |
| Lysine | FSCSVMHEALHNHYTQKSLSLSLG | |
| Ig light chain kappa | RTVAAPSVFIFPPSDEQLKSGTASVVCLLNNFYPREAKVQW | 387 |
| constant region (KCL) | KVDNALQSGNSQESVTEQDSKDSTYSLSSTLTLSKADYEKH | |
| | KVYACEVTHQGLSSPVTKSENRGEC | |
| Ig light chain kappa | GQPKANPTVTLFPPSSEELQANKATLVCLISDFYPGAVTVA | 388 |
| constant region (ACL) | WKADGSPVKAGVETTKPSKQSNNKYAASSYLSLTPEQWKSH | |
| | RSYSCQVTHEGSTVEKTVAPTECS | |

In some embodiments, the amino acid sequence of the heterologous polypeptide comprises an amino acid sequence set forth in Table 4. In some embodiments, the amino acid sequence of the heterologous polypeptide comprises or consists of an amino acid sequence set forth in Table 4, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises an amino acid sequence set forth in Table 4, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 4, comprising about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 4, consisting of about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 4, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous polypeptide consists of an amino acid sequence set forth in Table 4. In some embodiments, the amino acid sequence of the heterologous polypeptide consists of an amino acid sequence set forth in Table 4, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide consists of an amino acid sequence set forth in Table 4, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 4, comprising about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 4, consisting of about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 4, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous polypeptide comprises the amino acid sequence set forth in any one of SEQ ID NOS: 368-373 or 379-386. In some embodiments, the amino acid sequence of the heterologous polypeptide comprises the amino acid sequence set forth in any one of SEQ ID NOS: 368-373 or 379-386, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises the amino acid sequence set forth in any one of SEQ ID NOS: 368-373 or 379-386, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises the amino acid sequence set forth in any one of SEQ ID NOS: 368-373 or 379-386, comprising about 1, 2, 3, 4, 5, 6, 7, 8, 9, or 10 amino acid variations (e.g., amino acid substitutions, deletions, or additions). In some embodiments, the amino acid sequence of the heterologous polypeptide comprises the amino acid sequence set forth in any one of SEQ ID NOS: 368-373 or 379-386, consisting of about 1, 2, 3, 4, 5, 6, 7, 8, 9, or 10 amino acid variations (e.g., amino acid substitutions, deletions, or additions). In some embodiments, the amino acid sequence of the heterologous polypeptide comprises the amino acid sequence set forth in any one of SEQ ID NOS: 368-373 or 379-386, comprising no more than about 1, 2, 3, 4, 5, 6, 7, 8, 9, or 10 amino acid variations (e.g., amino acid substitutions, deletions, or additions).

In some embodiments, the amino acid sequence of the heterologous polypeptide consists of the amino acid sequence set forth in any one of SEQ ID NOS: 368-373 or 379-386. In some embodiments, the amino acid sequence of the heterologous polypeptide consists of the amino acid sequence set forth in any one of SEQ ID NOS: 368-373 or 379-386, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide consists of the amino acid sequence set forth in any one of SEQ ID NOS: 368-373 or 379-386, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of the amino acid sequence set forth in any one of SEQ ID NOS: 368-373 or 379-386, comprising about 1, 2, 3, 4, 5, 6, 7, 8, 9, or 10 amino acid variations (e.g., amino acid substitutions, deletions, or additions). In some embodiments, the amino acid sequence of the heterologous polypeptide consists of the amino acid sequence set forth in any one of SEQ ID NOS: 368-373 or 379-386, consisting of about 1, 2, 3, 4, 5, 6, 7, 8, 9, or 10 amino acid variations (e.g., amino acid substitutions, deletions, or additions). In some embodiments, the amino acid sequence of the heterologous polypeptide consists of the amino acid sequence set forth in any one of SEQ ID NOS: 368-373 or 379-386, comprising no more than about 1, 2, 3, 4, 5, 6, 7, 8, 9, or 10 amino acid variations (e.g., amino acid substitutions, deletions, or additions).

In some embodiments, the heterologous polypeptide comprises one or more mIg heavy chain constant regions (e.g., a CH2 region, a CH3 region, a hinge region, an Fc region). In some embodiments, the mIg is mIgG (mIgG). In some embodiments, the mIgG is mIgG1, mIgG2a, mIgG2c, mIgG2b, or mIgG3. In some embodiments, the mIgG is mIgG1 or mIgG2a. In some embodiments, the mIgG is mIgG1. In some embodiments, the mIgG is mIgG2a.

In some embodiments, the heterologous polypeptide comprises an mIgG CH2 region and an mIgG CH3 region. In some embodiments, the heterologous polypeptide comprises a partial mIgG hinge region, mIgG CH2 region, and mIgG CH3 region. In some embodiments, the heterologous polypeptide comprises an mIgG hinge region, mIgG CH2 region, and mIgG CH3 region. In some embodiments, the heterologous polypeptide comprises an mIgG1 CH2 region and an mIgG1 CH3 region. In some embodiments, the heterologous polypeptide comprises a partial mIgG1 hinge region, mIgG1 CH2 region, and mIgG1 CH3 region. In some embodiments, the heterologous polypeptide comprises an mIgG1 hinge region, mIgG1 CH2 region, and mIgG1 CH3 region. In some embodiments, the heterologous polypeptide comprises an mIgG2a CH2 region and an mIgG2a CH3 region. In some embodiments, the heterologous polypeptide comprises a partial mIgG2a hinge region, mIg2a CH2 region, and mIgG2a CH3 region. In some embodiments, the heterologous polypeptide comprises an mIgG2a hinge region, mIgG2a CH2 region, and mIgG2a CH3 region.

In some embodiments, the heterologous polypeptide consists of an mIgG CH2 region and an mIgG CH3 region. In some embodiments, the heterologous polypeptide consists of a partial mIgG hinge region, mIgG CH2 region, and mIgG CH3 region. In some embodiments, the heterologous polypeptide consists of an mIgG hinge region, mIgG CH2 region, and mIgG CH3 region. In some embodiments, the heterologous polypeptide consists of an mIgG1 CH2 region and an mIgG1 CH3 region. In some embodiments, the heterologous polypeptide consists of a partial mIgG1 hinge region, mIgG1 CH2 region, and mIgG1 CH3 region. In some embodiments, the heterologous polypeptide consists of an mIgG1 hinge region, mIgG1 CH2 region, and mIgG1 CH3 region. In some embodiments, the heterologous polypeptide consists of an mIgG2a CH2 region and an mIgG2a CH3 region. In some embodiments, the heterologous polypeptide consists of a partial mIgG2a hinge region, mIg2a CH2 region, and mIgG2a CH3 region. In some embodiments, the heterologous polypeptide comprises an mIgG2a hinge region, mIgG2a CH2 region, and mIgG2a CH3 region.

In some embodiments, the heterologous polypeptide comprises an mIg Fc region. In some embodiments, the mIg Fc region comprises at least a portion of a hinge region, a CH2 region, and a CH3 region. In some embodiments, the mIg Fc region comprises a hinge region, a CH2 region, and a CH3 region. In some embodiments, the mIg Fc region comprises at least a portion of an mIgG hinge region, an mIgG CH2 region, and an mIgG CH3 region. In some embodiments, the mIg Fc region comprises an mIgG hinge region, an mIgG CH2 region, and an mIgG CH3 region. In some embodiments, the mIg Fc region comprises at least a portion of an mIgG1 hinge region, an mIgG1 CH2 region, and an mIgG1 CH3 region. In some embodiments, the mIg Fc region comprises an mIgG1 hinge region, an mIgG1 CH2 region, and an mIgG1 CH3 region. In some embodiments, the mIg Fc region comprises at least a portion of an mIgG2a hinge region, an mIgG2a CH2 region, and an mIgG2a CH3 region. In some embodiments, the mIg Fc region comprises an mIgG2a hinge region, an mIgG2a CH2 region, and an mIgG2a CH3 region.

In some embodiments, the heterologous polypeptide consists of an mIg Fc region. In some embodiments, the mIg Fc region consists of at least a portion of a hinge region, a CH2 region, and a CH3 region. In some embodiments, the mIg Fc region consists of a hinge region, a CH2 region, and a CH3 region. In some embodiments, the mIg Fc region consists of at least a portion of an mIgG hinge region, an mIgG CH2 region, and an mIgG CH3 region. In some embodiments, the mIg Fc region consists of an mIgG hinge region, an mIgG CH2 region, and an mIgG CH3 region. In some embodiments, the mIg Fc region consists of at least a portion of an mIgG1 hinge region, an mIgG1 CH2 region, and an mIgG1 CH3 region. In some embodiments, the mIg Fc region consists of an mIgG1 hinge region, an mIgG1 CH2 region, and an mIgG1 CH3 region. In some embodiments, the mIg Fc region consists of at least a portion of an mIgG2a hinge region, an mIgG2a CH2 region, and an mIgG2a CH3 region. In some embodiments, the mIg Fc region consists of an mIgG2a hinge region, an mIgG2a CH2 region, and an mIgG2a CH3 region.

The amino acid sequence of exemplary reference mIgG1 and mIgG2a heavy chain constant regions, which can be incorporated in one or more of the embodiments described herein (e.g., fusion proteins and polypeptide), is provided in Table 11.

TABLE 11
The Amino Acid Sequence of Exemplary mIg chain constant region components.

| | | SEQ |
|---|---|---|
| Description | Amino Acid Sequence | ID NO |
| mIgG1 Hinge Region | VPRDCGCKPCICT | 470 |
| mIgG1 CH2 Region | VPEVSSVFIFPPKPKDVLTITLTPKVTCVVVAISKDDPEVQFSW | 471 |
| | FVDDVEVHTAQTQPREEQENSTERSVSELPIMHQDWLNGKEFKC | |
| | RVNSAAFPAPIEKTISKTK | |
| mIgG1 CH3 Region | GRPKAPQVYTIPPPKEQMAKDKVSLTCMITDFFPEDITVEWQWN | 472 |
| With C-terminal Lysine | GQPAENYKNTQPIMNINGSYFVYSKLNVQKSNWEAGNTFTCSVL | |
| | HEGLHNHHTEKSLSHSPGK | |
| mIgG1 CH3 Region | GRPKAPQVYTIPPPKEQMAKDKVSLTCMITDFFPEDITVEWQWN | 473 |
| Without C-terminal | GQPAENYKNTQPIMNINGSYFVYSKLNVQKSNWEAGNTFTCSVL | |
| Lysine | HEGLHNHHTEKSLSHSPG | |
| mIgG1 CH2 Region + | VPEVSSVFIFPPKPKDVLTITLTPKVTCVVVAISKDDPEVQFSW | 474 |
| CH3 Region | FVDDVEVHTAQTQPREEQENSTERSVSELPIMHQDWLNGKEFKC | |
| With C-terminal Lysine | RVNSAAFPAPIEKTISKTKGRPKAPQVYTIPPPKEQMAKDKVSL | |
| | TCMITDFFPEDITVEWQWNGQPAENYKNTQPIMNINGSYFVYSK | |
| | LNVQKSNWEAGNTFTCSVLHEGLHNHHTEKSLSHSPGK | |
| mIgG1 CH2 Region + | VPEVSSVFIFPPKPKDVLTITLTPKVTCVVVAISKDDPEVQFSW | 475 |
| CH3 Region | FVDDVEVHTAQTQPREEQFNSTERSVSELPIMHQDWLNGKEFKC | |
| Without C-terminal | RVNSAAFPAPIEKTISKTKGRPKAPQVYTIPPPKEQMAKDKVSL | |
| Lysine | TCMITDFFPEDITVEWQWNGQPAENYKNTQPIMNINGSYFVYSK | |
| | LNVQKSNWEAGNTFTCSVLHEGLHNHHTEKSLSHSPG | |
| mIgG1 Hinge Region + | VPRDCGCKPCICTVPEVSSVFIFPPKPKDVLTITLTPKVTCVVV | 476 |
| CH2 Region + CH3 | AISKDDPEVQFSWFVDDVEVHTAQTQPREEQENSTERSVSELPI | |
| Region | MHQDWLNGKEFKCRVNSAAFPAPIEKTISKTKGRPKAPQVYTIP | |
| With C-terminal Lysine | PPKEQMAKDKVSLTCMITDFFPEDITVEWQWNGQPAENYKNTQP | |
| | IMNTNGSYFVYSKLNVQKSNWEAGNTFTCSVLHEGLHNHHTEKS | |
| | LSHSPGK | |
| mIgG1 Hinge Region + | VPRDCGCKPCICTVPEVSSVFIFPPKPKDVLTITLTPKVTCVVV | 477 |
| CH2 Region + CH3 | AISKDDPEVQFSWFVDDVEVHTAQTQPREEQENSTERSVSELPI | |
| Region | MHQDWLNGKEFKCRVNSAAFPAPIEKTISKTKGRPKAPQVYTIP | |
| Without C-terminal | PPKEQMAKDKVSLTCMITDFFPEDITVEWQWNGQPAENYKNTQP | |
| Lysine | IMNTNGSYFVYSKLNVQKSNWEAGNTFTCSVLHEGLHNHHTEKS | |
| | LSHSPGK | |
| mIgG2a Hinge Region | EPRGPTIKPCPPCKCP | 478 |
| mIgG2a CH2 Region | APNAAGGPSVFIFLLKIKDVLMISLSPIVTCVVVDVSEDDPDVQ | 479 |
| | ISWFVNNVEVHTAQTQTHREDYNSTLRVVSALPIQHQDWMSGKE | |
| | FKCKVNNKDLPAPIERTISKPKGSVRAPQVYVLPPPEEEMTKKQ | |
| | VT | |
| mIgG2a CH3 Region | LTCMVTDEMPEDIYVEWTNNGKTELNYKNTEPVLDSDGSYFMYS | 480 |
| With C-terminal Lysine | KLRVEKKNWVERNSYSCSVVHEGLHNHHTTKSFSRTPGK | |
| mIgG2a CH3 Region | LTCMVTDEMPEDIYVEWTNNGKTELNYKNTEPVLDSDGSYFMYS | 481 |
| Without C-terminal | KLRVEKKNWVERNSYSCSVVHEGLHNHHTTKSESRTPG | |
| Lysine | | |
| mIgG2a CH2 Region + | APNAAGGPSVFIFLLKIKDVLMISLSPIVTCVVVDVSEDDPDVQ | 482 |
| CH3 Region | ISWFVNNVEVHTAQTQTHREDYNSTLRVVSALPIQHQDWMSGKE | |
| With C-terminal Lysine | FKCKVNNKDLPAPIERTISKPKGSVRAPQVYVLPPPEEEMTKKQ | |
| | VTLTCMVTDEMPEDIYVEWTNNGKTELNYKNTEPVLDSDGSYEM | |
| | YSKLRVEKKNWVERNSYSCSVVHEGLHNHHTTKSFSRTPGK | |
| mIgG2a CH2 Region + | APNAAGGPSVFIFLLKIKDVLMISLSPIVTCVVVDVSEDDPDVQ | 483 |
| CH3 Region | ISWFVNNVEVHTAQTQTHREDYNSTLRVVSALPIQHQDWMSGKE | |
| Without C-terminal | FKCKVNNKDLPAPIERTISKPKGSVRAPQVYVLPPPEEEMTKKQ | |
| Lysine | VTLTCMVTDEMPEDIYVEWTNNGKTELNYKNTEPVLDSDGSYEM | |
| | YSKLRVEKKNWVERNSYSCSVVHEGLHNHHTTKSFSRTPG | |
| mIgG2a Hinge Region + | EPRGPTIKPCPPCKCPAPNAAGGPSVFIFLLKIKDVLMISLSPI | 484 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| With C-terminal Lysine | QVYVLPPPEEEMTKKQVTLTCMVTDEMPEDIYVEWTNNGKTELN | |
| | YKNTEPVLDSDGSYFMYSKLRVEKKNWVERNSYSCSVVHEGLHN | |
| | HHTTKSFSRTPGK | |
| mIgG2a Hinge Region + | EPRGPTIKPCPPCKCPAPNAAGGPSVFIFLLKIKDVLMISLSPI | 485 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| Without C-terminal | QVYVLPPPEEEMTKKQVTLTCMVTDEMPEDIYVEWTNNGKTELN | |
| Lysine | YKNTEPVLDSDGSYFMYSKLRVEKKNWVERNSYSCSVVHEGLHN | |
| | HHTTKSFSRTPG | |

In some embodiments, the amino acid sequence of the heterologous polypeptide comprises an amino acid sequence set forth in Table 11. In some embodiments, the amino acid sequence of the heterologous polypeptide comprises an amino acid sequence set forth in Table 11, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises an amino acid sequence set forth in Table 11, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 11, comprising about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 11, comprising about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises an amino acid sequence set forth in Table 11, consisting of about no more than 1, 2, 3, 4, 5, 6, 7, 8, 9, 10, or more amino acid variations (e.g., amino acid substitutions, deletions, or additions).

In some embodiments, the amino acid sequence of the heterologous polypeptide consists of an amino acid sequence set forth in Table 11. In some embodiments, the amino acid sequence of the heterologous polypeptide consists of an amino acid sequence set forth in Table 11, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide consists of an amino acid sequence set forth in Table 11, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 11, comprising about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 11, consisting of about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of an amino acid sequence set forth in Table 11, comprising no more than about 1, 2, 3, 4, 5, 6, 7, 8, 9, 10, or more amino acid variations (e.g., amino acid substitutions, deletions, or additions).

In some embodiments, the amino acid sequence of the heterologous polypeptide comprises the amino acid sequence set forth in any one of SEQ ID NOS: 474-477 or 482-485. In some embodiments, the amino acid sequence of the heterologous polypeptide comprises the amino acid sequence set forth in any one of SEQ ID NOS: 474-477 or 482-485, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises the amino acid sequence set forth in any one of SEQ ID NOS: 474-477 or 482-485, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises the amino acid sequence set forth in any one of SEQ ID NOS: 474-477 or 482-485, comprising about 1, 2, 3, 4, 5, 6, 7, 8, 9, or 10 amino acid variations (e.g., amino acid substitutions, deletions, or additions). In some embodiments, the amino acid sequence of the heterologous polypeptide comprises the amino acid sequence set forth in any one of SEQ ID NOS: 474-477 or 482-485, consisting of about 1, 2, 3, 4, 5, 6, 7, 8, 9, or 10 amino acid variations (e.g., amino acid substitutions, deletions, or additions). In some embodiments, the amino acid sequence of the heterologous polypeptide comprises or consists of the amino acid sequence set forth in any one of SEQ ID NOS: 474-477 or 482-485, comprising no more than about 1, 2, 3, 4, 5, 6, 7, 8, 9, or 10 amino acid variations (e.g., amino acid substitutions, deletions, or additions).

In some embodiments, the amino acid sequence of the heterologous polypeptide consists of the amino acid sequence set forth in any one of SEQ ID NOS: 474-477 or 482-485. In some embodiments, the amino acid sequence of the heterologous polypeptide consists of the amino acid sequence set forth in any one of SEQ ID NOS: 474-477 or 482-485, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide consists of the amino acid sequence set forth in any one of SEQ ID NOS: 474-477 or 482-485, comprising at least about 1, 2, 3, 4, 5, 6, 7, 8, 9, 10, or more amino acid variations (e.g., amino acid substitutions, deletions, or additions). In some embodiments, the amino acid sequence of the heterologous polypeptide consists of the amino acid sequence set forth in any one of SEQ ID NOS: 474-477 or 482-485, comprising about 1, 2, 3, 4, 5, 6, 7, 8, 9, or 10 amino acid variations (e.g., amino acid substitutions, deletions, or additions). In some embodiments, the amino acid sequence of the heterologous polypeptide consists of the amino acid sequence set forth in any one of SEQ ID NOS: 474-477 or 482-485, consisting of about 1, 2, 3, 4, 5, 6, 7, 8, 9, or 10 amino acid variations (e.g., amino acid substitutions, deletions, or additions). In some embodiments, the amino acid sequence of the heterologous polypeptide consists of the amino acid sequence set forth in any one of SEQ ID NOS: 474-477 or 482-485, comprising no more than about 1, 2, 3, 4, 5, 6, 7, 8, 9, or 10 amino acid variations (e.g., amino acid substitutions, deletions, or additions).

### 5.10.1.1 Ig Effector Function

In some embodiments, the Ig (e.g., hIg, mIg) Fc region of a fusion protein described herein exhibits a decrease in one or more Fe effector function relative to a reference (e.g., wild type) Ig (e.g., hIg, mIg) Fc region. Exemplary Ig (e.g., hIg, mIg) Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Fc effector function, including, any one or more of ADCC, CDC, ADCP, Fc receptor (e.g., Fc receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4,3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of an Ig fusion protein described herein to bind C1q (or bind with less affinity than a reference fusion protein) and hence lack (or have decreased) CDC activity. The binding of a hIg fusion protein described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of an Ig fusion protein described herein to an Ig Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4” Ed., Lippincott-Raven, Philadelphia (1999), the entire contents of which is incorporated by reference herein for all purposes.

In some embodiments, the Ig (e.g., hIg, mIg) Fc region of a fusion protein described herein is varied (e.g., comprises one or more variation (e.g., one or more amino acid substitution, deletion, addition, etc.)). In some embodiments, the one or more variation (e.g., the one or more amino acid substitution, deletion, addition, etc.)) decreases or abolishes one or more Fc effector function, relative to a reference hIg Fc that does not comprise the one or more variation (e.g., the one or more amino acid substitution, deletion, addition, etc.)).

In some embodiments, the variant Ig (e.g., hIg, mIg) Fc fusion protein exhibits no detectable or decreased ADCC compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits no detectable or decreased CDC compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits no detectable or decreased ADCP compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))) compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to FcγRI, FcγIIa, and/or FcγIIIa compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to FcγRI compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to FcγIIa compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to FcγIIIa compared to a reference fusion protein that does not comprise the Ig (e.g., hIg, mIg) Fc variation (e.g., the one or more variation (e.g., one or more amino acid substitution, deletion, or addition)). In some embodiments, the variant Ig (e.g., hIg, mIg) Fc fusion protein exhibits decreased or no binding affinity to C1q compared to a reference fusion protein that does not comprise the Ig (e.g., hIg, mIg) Fc variation (e.g., the one or more variation (e.g., one or more amino acid substitution, deletion, or addition)).

Amino acid substitutions that decrease or abolish one or more Ig (e.g., hIg, mIg) Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

In some embodiments, the variant Ig Fc fusion protein comprises a hIg Fc region comprising one or more amino acid variation. In some embodiments, the variant hIg Fc fusion protein comprises a hIg4 Fc region comprising one or more amino acid variation. In some embodiments, the hIgG4 Fc region comprises an amino acid substitution at amino acid positions S228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

In some embodiments, the hIg Fc fusion protein comprises a hIgG1 Fc region comprising one or more amino acid variations. In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

The amino acid sequence of exemplary variant hIg Fc regions that are known in the art to exhibit a decrease in one more Fc effector function is provided in Table 5.

TABLE 5
The Amino Acid Sequence of Exemplary Variant hIg Fc Regions.

| | | SEQ |
|---|---|---|
| Description | Amino Acid Sequence | ID NO |
| hIgG1 CH2 Region + | PCPAPEAAGGPSVFLFPPKPKDTLMISRTPEVTCVVVDVSHED | 389 |
| CH3 Region | PEVKFNWYVDGVEVHNAKTKPREEQYNSTYRVVSVLTVLHQDW | |
| L234A/L235A | LNGKEYKCKVSNKALPAPIEKTISKAKGQPREPQVYTLPPSRD | |
| With C-terminal Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 390 |
| CH3 Region | PEVKFNWYVDGVEVHNAKTKPREEQYNSTYRVVSVLTVLHQDW | |
| L234A/L235A | LNGKEYKCKVSNKALPAPIEKTISKAKGQPREPQVYTLPPSRD | |
| Without C-terminal | ELTKNQVSLTCLVKGFYPSDIAVEWESNGQPENNYKTTPPVLD | |
| Lysine | SDGSFFLYSKLTVDKSRWQQGNVFSCSVMHEALHNHYTOKSLS | |
| | LSPG | |
| hIgG1 Partial Hinge | TCPPCPAPEAAGGPSVFLFPPKPKDTLMISRTPEVTCVVVDVS | 391 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| L234A/L235A | SRDELTKNQVSLTCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPEAAGGPSVFLFPPKPKDTLMISRTPEVTCVVVDVS | 392 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| L234A/L235A | SRDELTKNQVSLTCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPEAAGGPSVFLFPPKPKDTLMISRTPE | 393 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPEAAGGPSVFLFPPKPKDTLMISRTPE | 394 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGOP | |
| Without C-terminal | ENNYKTTPPVLDSDGSFFLYSKLTVDKSRWQQGNVFSCSVMHE | |
| Lysine | ALHNHYTQKSLSLSPG | |
| hIgG4 CH2 Region + | APEAAGGPSVFLFPPKPKDTLMISRTPEVTCVVVDVSQEDPEV | 395 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| With C-terminal Lysine | KNQVSLTCLVKGFYPSDIAVEWESNGQPENNYKTTPPVLDSDG | |
| | SFFLYSRLTVDKSRWQEGNVESCSVMHEALHNHYTQKSLSLSL | |
| | GK | |
| hIgG4 CH2 Region + | APEAAGGPSVFLFPPKPKDTLMISRTPEVTCVVVDVSQEDPEV | 396 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| Without C-terminal | KNQVSLTCLVKGFYPSDIAVEWESNGQPENNYKTTPPVLDSDG | |
| Lysine | SFFLYSRLTVDKSRWQEGNVFSCSVMHEALHNHYTQKSLSLSL | |
| | G | |
| hIgG4 Partial Hinge | PCPSCPAPEAAGGPSVFLFPPKPKDTLMISRTPEVTCVVVDVS | 397 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKTTPP | |
| With C-terminal Lysine | VLDSDGSFFLYSRLTVDKSRWQEGNVESCSVMHEALHNHYTQK | |
| | SLSLSLGK | |
| hIgG4 Partial Hinge | PCPPCPAPEAAGGPSVFLFPPKPKDTLMISRTPEVTCVVVDVS | 398 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKTTPP | |
| Without C-terminal | VLDSDGSFFLYSRLTVDKSRWQEGNVFSCSVMHEALHNHYTQK | |
| Lysine | SLSLSLG | |
| hIgG4 Hinge Region + | ESKYGPPCPPCPAPEAAGGPSVFLFPPKPKDTLMISRTPEVTC | 399 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| S228P/F234A/L235A | VYTLPPSQEEMTKNQVSLTCLVKGFYPSDIAVEWESNGQPENN | |
| With C-terminal Lysine | YKTTPPVLDSDGSFFLYSRLTVDKSRWQEGNVESCSVMHEALH | |
| | NHYTQKSLSLSLGK | |
| hIgG4 Hinge Region + | ESKYGPPCPPCPAPEAAGGPSVFLFPPKPKDTLMISRTPEVTC | 400 |
| CH2 Region + CH3 | VVVDVSQEDPEVQFNWYVDGVEVHNAKTKPREEQENSTYRVVS | |
| Region | VLTVLHQDWLNGKEYKCKVSNKGLPSSIEKTISKAKGQPREPQ | |
| S228P/F234A/L235A | VYTLPPSQEEMTKNQVSLTCLVKGFYPSDIAVEWESNGQPENN | |
| Without C-terminal | YKTTPPVLDSDGSFFLYSRLTVDKSRWQEGNVESCSVMHEALH | |
| Lysine | NHYTQKSLSLSLG | |
| hIgG4 Hinge Region + | AESKYGPPCPPCPAPEAAGGPSVFLFPPKPKDTLMISRTPEVT | 40 |
| CH2 Region + CH3 | CVVVDVSQEDPEVQFNWYVDGVEVHNAKTKPREEQENSTYRVV | |
| Region | SVLTVLHQDWLNGKEYKCKVSNKGLPSSIEKTISKAKGQPREP | |
| Variant | QVYTLPPSQEEMTKNQVSLTCLVKGFYPSDIAVEWESNGQPEN | |
| S228P/F234A/L235A | NYKTTPPVLDSDGSFFLYSRLTVDKSRWQEGNVFSCSVMHEAL | |
| With C-terminal Lysine | HNHYTQKSLSLSLGK | |
| hIgG4 Hinge Region + | AESKYGPPCPPCPAPEAAGGPSVFLFPPKPKDTLMISRTPEVT | 402 |
| CH2 Region + CH3 | CVVVDVSQEDPEVQFNWYVDGVEVHNAKTKPREEQENSTYRVV | |
| Region | SVLTVLHQDWLNGKEYKCKVSNKGLPSSIEKTISKAKGQPREP | |
| Variant | QVYTLPPSQEEMTKNQVSLTCLVKGFYPSDIAVEWESNGQPEN | |
| S228P/F234A/L235A | NYKTTPPVLDSDGSFFLYSRLTVDKSRWQEGNVESCSVMHEAL | |
| Without C-terminal | HNHYTQKSLSLSLG | |
| Lysine | | |

In some embodiments, the variant hIg Fe fusion protein comprises a hIg Fe region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 5.

In some embodiments, the amino acid sequence of the variant hIg Fc fusion protein comprises a hIg Fc region that comprises the amino acid sequence of a polypeptide set forth in Table 5, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that comprises the amino acid sequence of a polypeptide set forth in Table 5, and further comprises or consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence of a polypeptide set forth in Table 5, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence of a polypeptide set forth in Table 5, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence of a polypeptide set forth in Table 5, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the variant hIg Fc fusion protein comprises a hIg Fc region that consists of the amino acid sequence of a polypeptide set forth in Table 5, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that consists of the amino acid sequence of a polypeptide set forth in Table 5, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence of a polypeptide set forth in Table 5, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence of a polypeptide set forth in Table 5, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence of a polypeptide set forth in Table 5, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the variant hIg Fc fusion protein comprises a hIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 389-402.

In some embodiments, the amino acid sequence of the variant hIg Fc fusion protein comprises a hIg Fc region that comprises the amino acid sequence set forth in any one of SEQ ID NOS: 389-402, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that comprises the amino acid sequence set forth in any one of SEQ ID NOS: 389-402, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence set forth in any one of SEQ ID NOS: 389-402, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence set forth in any one of SEQ ID NOS: 389-402, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that comprises the amino acid sequence set forth in any one of SEQ ID NOS: 389-402, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the variant hIg Fc fusion protein comprises a hIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 389-402.

In some embodiments, the amino acid sequence of the variant hIg Fc fusion protein comprises a hIg Fc region that consists of the amino acid sequence set forth in any one of SEQ ID NOS: 389-402, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein comprises a hIg Fc region that consists of the amino acid sequence set forth in any one of SEQ ID NOS: 389-402, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence set forth in any one of SEQ ID NOS: 389-402, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence set forth in any one of SEQ ID NOS: 389-402, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant hIg Fc fusion protein comprises a hIg Fc region that consists of the amino acid sequence set forth in any one of SEQ ID NOS: 389-402, and further comprises no more than about 1, 2, 3, 4, 5, 6, 7, 8, 9, or 10 amino acid variations (e.g., substitutions, additions, deletions, etc. (e.g., substitutions)).

In some embodiments, the variant mIg Fc fusion protein comprises an mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234P and/or L235P, EU numbering according to Kabat. In some embodiments, the mIgG2a Fc region comprises the following amino acid substitutions L234P, L235P, and P329G, EU numbering according to Kabat. In some embodiments, the mIgG2a Fc region comprises the following amino acid substitutions L234P, L235P, and P329A, EU numbering according to Kabat.

In some embodiments, the variant mIg Fc fusion protein comprises an mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234A and/or L235A, EU numbering according to Kabat. In some embodiments, the mIgG2a Fc region comprises the following amino acid substitutions L234A, L235A, and P329G, EU numbering according to Kabat. In some embodiments, the mIgG2a Fc region comprises the following amino acid substitutions L234A, L235A, and P329A, EU numbering according to Kabat.

The amino acid sequence of exemplary variant hIg Fc regions that are known in the art to exhibit a decrease in one more effector function is provided in Table 12.

TABLE 12
The amino acid sequence of exemplary variant mIg Fc Regions.

| | | SEQ |
|---|---|---|
| Description | Amino Acid Sequence | ID NO |
| mIgG2a CH2 Region + | APNAAGGPSVFIFPPKIKDVLMISLSPIVTCVVVDVSEDDPDV | 486 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| With C-terminal Lysine | KKQVTLTCMVTDEMPEDIYVEWINNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFPPKIKDVLMISLSPIVTCVVVDVSEDDPDV | 487 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| Without C-terminal | KKQVTLTCMVTDEMPEDIYVEWTNNGKTELNYKNTEPVLDSDG | |
| Lysine | SYFMYSKLRVEKKNWVERNSYSCSVVHEGLHNHHTTKSFSRTP | |
| | G | |
| mIgG2a Hinge Region + | EPRGPTIKPCPPCKCPAPNAAGGPSVFIFPPKIKDVLMISLSP | 488 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P/P329G | RAPQVYVLPPPEEEMTKKQVTLTCMVTDEMPEDIYVEWTNNGK | |
| With C-terminal Lysine | TELNYKNTEPVLDSDGSYFMYSKLRVEKKNWVERNSYSCSVVH | |
| | EGLHNHHTTKSFSRTPGK | |
| mIgG2a Hinge Region + | EPRGPTIKPCPPCKCPAPNAAGGPSVFIFPPKIKDVLMISLSP | 489 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P/P329G | RAPQVYVLPPPEEEMTKKQVTLTCMVTDEMPEDIYVEWTNNGK | |
| Without C-terminal | TELNYKNTEPVLDSDGSYFMYSKLRVEKKNWVERNSYSCSVVH | |
| Lysine | EGLHNHHTTKSFSRTPG | |
| mIgG2a CH2 Region + | APNAAGGPSVFIFAAKIKDVLMISLSPIVTCVVVDVSEDDPDV | 490 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| With C-terminal Lysine | KKQVTLTCMVTDEMPEDIYVEWTNNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFAAKIKDVLMISLSPIVTCVVVDVSEDDPDV | 491 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| Without C-terminal | KKQVTLTCMVTDEMPEDIYVEWTNNGKTELNYKNTEPVLDSDG | |
| Lysine | SYFMYSKLRVEKKNWVERNSYSCSVVHEGLHNHHTTKSESRTP | |
| | G | |
| mIgG2a Hinge Region + | EPRGPTIKPCAACKCPAPNAAGGPSVFIFPPKIKDVLMISLSP | 492 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDEMPEDIYVEWTNNGK | |
| With C-terminal Lysine | TELNYKNTEPVLDSDGSYFMYSKLRVEKKNWVERNSYSCSVVH | |
| | EGLHNHHTTKSFSRTPGK | |
| mIgG2a Hinge Region + | EPRGPTIKPCAACKCPAPNAAGGPSVFIFPPKIKDVLMISLSP | 493 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDEMPEDIYVEWTNNGK | |
| Without C-terminal | TELNYKNTEPVLDSDGSYFMYSKLRVEKKNWVERNSYSCSVVH | |
| Lysine | EGLHNHHTTKSFSRTPG | |

In some embodiments, the variant mIg Fe fusion protein comprises a mIg Fe region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12.

In some embodiments, the amino acid sequence of the variant mIg Fc fusion protein comprises a mIg Fc region that comprises the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that comprises the amino acid sequence of a polypeptide set forth in Table 12, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence of a polypeptide set forth in Table 12, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence of a polypeptide set forth in Table 12, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence of a polypeptide set forth in Table 12, and further comprises no more than about 1, 2, 3, 4, 5, 6, 7, 8, 9, or 10 amino acid variations (e.g., substitutions, additions, deletions, etc. (e.g., substitutions)).

In some embodiments, the variant mIg Fc fusion protein comprises a mIg Fc region consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12.

In some embodiments, the amino acid sequence of the variant mIg Fc fusion protein comprises a mIg Fc region that consists of the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that consists of the amino acid sequence of a polypeptide set forth in Table 12, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence of a polypeptide set forth in Table 12, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence of a polypeptide set forth in Table 12, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence of a polypeptide set forth in Table 12, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the variant mIg Fc fusion protein or polypeptide comprises a mIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 486-493.

In some embodiments, the amino acid sequence of the variant mIg Fc fusion protein comprises a mIg Fc region that comprises the amino acid sequence set forth in any one of SEQ ID NOS: 486-493, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that comprises the amino acid sequence set forth in any one of SEQ ID NOS: 486-493, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence set forth in any one of SEQ ID NOS: 486-493, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence set forth in any one of SEQ ID NOS: 486-493, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that comprises the amino acid sequence set forth in any one of SEQ ID NOS: 486-493, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the variant mIg Fc fusion protein comprises a mIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence set forth in any one of SEQ ID NOS: 486-493.

In some embodiments, the amino acid sequence of the variant mIg Fc fusion protein comprises a mIg Fc region that consists of the amino acid sequence set forth in any one of SEQ ID NOS: 486-493, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein comprises a mIg Fc region that consists of the amino acid sequence set forth in any one of SEQ ID NOS: 486-493, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence set forth in any one of SEQ ID NOS: 486-493, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence set forth in any one of SEQ ID NOS: 486-493, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the variant mIg Fc fusion protein comprises a mIg Fc region that consists of the amino acid sequence set forth in any one of SEQ ID NOS: 486-493, and further comprises no more than about 1, 2, 3, 4, 5, 6, 7, 8, 9, or 10 amino acid variations (e.g., substitutions, additions, deletions, etc. (e.g., substitutions)).

### 5.10.2 Linkers

As described herein, the heterologous moiety (e.g., heterologous polypeptide) can be directly operably connected or indirectly operably connected to an agent (e.g., protein) described herein (e.g., an hIL-10R binding agent described herein (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)).

In some embodiments, the heterologous polypeptide is directly operably connected to a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)) via a peptide bond. In some embodiments, the heterologous polypeptide is indirectly operably connected to a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)) via a peptide linker.

In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2,3,4,5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises glycine, serine, or both glycine and serine amino acid residues. In some embodiments, the amino acid sequence of the peptide linker comprises glycine, serine, and proline amino acid residues. In some embodiments, the amino acid sequence of the peptide linker consists of glycine, serine, or both glycine and serine amino acid residues. In some embodiments, the amino acid sequence of the peptide linker consists of glycine, serine, and proline amino acid residues.

The amino acid sequence of exemplary peptide linkers, which can be incorporated in one or more of the embodiments described herein (e.g., fusion proteins), is set provided in Table 6.

TABLE 6
The Amino Acid Sequence of Exemplary Peptide
Linkers.

| Description | Amino Acid Sequence | SEQ ID NO |
|---|---|---|
| Linker A | GGGGGGGS | 403 |
| Linker B | GGGGGGGSGGGGGGGS | 404 |
| Linker C | GGGGGGGSGGGGGGGSGGGGGGGS | 405 |
| Linker D | GGGGS | 406 |
| Linker E | GGGGSGGGGS | 407 |
| Linker F | GGGGSGGGGSGGGGS | 408 |
| Linker G | GGGS | 409 |
| Linker H | GGGSGGGS | 410 |
| Linker I | GGGSGGGSGGGS | 411 |

In some embodiments, the amino acid sequence of the peptide linker comprises the amino acid sequence of any one of the linkers set forth in Table 6. In some embodiments, the amino acid sequence of the peptide linker comprises the amino acid sequence of any one of the linkers set forth in Table 6, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence of any one of the linkers set forth in Table 6, comprising 1, 2, or 3 amino acid variations (e.g., substitutions, deletions, additions). In some embodiments, the amino acid sequence of the peptide linker comprises the amino acid sequence of any one of the linkers set forth in Table 6, consisting of 1, 2, or 3 amino acid variations (e.g., substitutions, deletions, additions). In some embodiments, the amino acid sequence of the peptide linker comprises the amino acid sequence of any one of the linkers set forth in Table 6, and further comprises 1 or more but less than 15% (less than 12%, less than 10%, less than 8%), amino acid substitutions. In some embodiments, the amino acid sequence of the peptide linker comprises the amino acid sequence of any one of the linkers set forth in Table 6, comprising 1, 2, or 3 amino acid substitutions. In some embodiments, the amino acid sequence of the peptide linker comprises the amino acid sequence of any one of the linkers set forth in Table 6, consisting of 1, 2, or 3 amino acid substitutions.

In some embodiments, the amino acid sequence of the peptide linker consists of the amino acid sequence of any one of the linkers set forth in Table 6. In some embodiments, the amino acid sequence of the peptide linker consists of the amino acid sequence of any one of the linkers set forth in Table 6, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence of any one of the linkers set forth in Table 6, comprising 1, 2, or 3 amino acid variations (e.g., substitutions, deletions, additions). In some embodiments, the amino acid sequence of the peptide linker consists of the amino acid sequence of any one of the linkers set forth in Table 6, consisting of 1, 2, or 3 amino acid variations (e.g., substitutions, deletions, additions). In some embodiments, the amino acid sequence of the peptide linker consists of the amino acid sequence of any one of the linkers set forth in Table 6, and further comprises 1 or more but less than 15% (less than 12%, less than 10%, less than 8%), amino acid substitutions. In some embodiments, the amino acid sequence of the peptide linker consists of the amino acid sequence of any one of the linkers set forth in Table 6, comprising 1, 2, or 3 amino acid substitutions. In some embodiments, the amino acid sequence of the peptide linker consists of the amino acid sequence of any one of the linkers set forth in Table 6, consisting of 1, 2, or 3 amino acid substitutions.

In some embodiments, the amino acid sequence of the peptide linker comprises the amino acid sequence set forth in any one of SEQ ID NOS: 403-411. In some embodiments, the amino acid sequence of the peptide linker comprises the amino acid sequence set forth in any one of SEQ ID NOS: 403-411, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in any one of SEQ ID NOS: 403-411, comprising 1, 2, or 3 amino acid variations (e.g., substitutions, deletions, additions). In some embodiments, the amino acid sequence of the peptide linker comprises the amino acid sequence set forth in any one of SEQ ID NOS: 403-411, consisting of 1, 2, or 3 amino acid variations (e.g., substitutions, deletions, additions). In some embodiments, the amino acid sequence of the peptide linker comprises the amino acid sequence set forth in any one of SEQ ID NOS: 403-411, and further comprises 1 or more but less than 15% (less than 12%, less than 10%, less than 8%), amino acid substitutions. In some embodiments, the amino acid sequence of the peptide linker comprises the amino acid sequence set forth in any one of SEQ ID NOS: 403-411, comprising 1, 2, or 3 amino acid substitutions. In some embodiments, the amino acid sequence of the peptide linker comprises the amino acid sequence set forth in any one of SEQ ID NOS: 403-411, consisting of 1, 2, or 3 amino acid substitutions.

In some embodiments, the amino acid sequence of the peptide linker consists of the amino acid sequence set forth in any one of SEQ ID NOS: 403-411. In some embodiments, the amino acid sequence of the peptide linker consists of the amino acid sequence set forth in any one of SEQ ID NOS: 403-411, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in any one of SEQ ID NOS: 403-411, comprising 1, 2, or 3 amino acid variations (e.g., substitutions, deletions, additions). In some embodiments, the amino acid sequence of the peptide linker consists of the amino acid sequence set forth in any one of SEQ ID NOS: 403-411, consisting of 1, 2, or 3 amino acid variations (e.g., substitutions, deletions, additions). In some embodiments, the amino acid sequence of the peptide linker consists of the amino acid sequence set forth in any one of SEQ ID NOS: 403-411, and further comprises 1 or more but less than 15% (less than 12%, less than 10%, less than 8%), amino acid substitutions. In some embodiments, the amino acid sequence of the peptide linker consists of the amino acid sequence set forth in any one of SEQ ID NOS: 403-411, comprising 1, 2, or 3 amino acid substitutions. In some embodiments, the amino acid sequence of the peptide linker consists of the amino acid sequence set forth in any one of SEQ ID NOS: 403-411, consisting of 1, 2, or 3 amino acid substitutions.

In some embodiments, the amino acid sequence of the peptide linker comprises the amino acid sequence set forth in SEQ ID NO: 405, and further comprises 1 or more but less than 15% (less than 12%, less than 10%, less than 8%), amino acid substitutions. In some embodiments, the amino acid sequence of the peptide linker comprises the amino acid sequence set forth in SEQ ID NO: 405. In some embodiments, the amino acid sequence of the peptide linker comprises the amino acid sequence of any one of SEQ ID NO: 405, comprising 1, 2, or 3 amino acid variations (e.g., substitutions, additions, deletions). In some embodiments, the amino acid sequence of the peptide linker comprises the amino acid sequence of any one of SEQ ID NO: 405, comprising 1, 2, or 3 amino acid substitutions. In some embodiments, the amino acid sequence of the peptide linker comprises the amino acid sequence of any one of SEQ ID NO: 405, consisting of 1, 2, or 3 amino acid variations (e.g., substitutions, additions, deletions). In some embodiments, the amino acid sequence of the peptide linker comprises the amino acid sequence of any one of SEQ ID NO: 405, consisting of 1, 2, or 3 amino acid substitutions.

In some embodiments, the amino acid sequence of the peptide linker consists of the amino acid sequence set forth in SEQ ID NO: 405, and further comprises 1 or more but less than 15% (less than 12%, less than 10%, less than 8%), amino acid substitutions. In some embodiments, the amino acid sequence of the peptide linker consists of the amino acid sequence set forth in SEQ ID NO: 405. In some embodiments, the amino acid sequence of the peptide linker consists of the amino acid sequence of any one of SEQ ID NO: 405, comprising 1, 2, or 3 amino acid variations (e.g., substitutions, additions, deletions). In some embodiments, the amino acid sequence of the peptide linker consists of the amino acid sequence of any one of SEQ ID NO: 405, comprising 1, 2, or 3 amino acid substitutions. In some embodiments, the amino acid sequence of the peptide linker consists of the amino acid sequence of any one of SEQ ID NO: 405, consisting of 1, 2, or 3 amino acid variations (e.g., substitutions, additions, deletions). In some embodiments, the amino acid sequence of the peptide linker consists of the amino acid sequence of any one of SEQ ID NO: 405, consisting of 1, 2, or 3 amino acid substitutions.

### 5.10.3 Orientation

The heterologous moiety (e.g., heterologous polypeptide) and the protein described herein (e.g., the hIL-10R binding agent (e.g., the hIL-10R binding protein (or the functional fragment and/or functional variant thereof) or the nucleic acid molecule encoding the hIL-10R binding protein (or the functional fragment and/or functional variant thereof)), the IGIP (e.g., hIGIP) protein (or the functional fragment and/or functional variant thereof) or the nucleic acid molecule encoding the IGIP protein (or the functional fragment and/or functional variant thereof)); and/or the immunogen (e.g., the immunogenic protein (or the functional fragment and/or functional variant thereof) or the nucleic acid molecule encoding the immunogenic protein (or the functional fragment and/or functional variant thereof)) can be arranged in any orientation so long as the protein described herein ((e.g., the hIL-10R binding agent (e.g., the hIL-10R binding protein (or the functional fragment and/or functional variant thereof) or the nucleic acid molecule encoding the hIL-10R binding protein (or the functional fragment and/or functional variant thereof)), the IGIP (e.g., hIGIP) protein (or the functional fragment and/or functional variant thereof) or the nucleic acid molecule encoding the IGIP protein (or the functional fragment and/or functional variant thereof)); and/or the immunogen (e.g., the immunogenic protein (or the functional fragment and/or functional variant thereof) or the nucleic acid molecule encoding the immunogenic protein (or the functional fragment and/or functional variant thereof)) maintains the ability to mediate its function and in the embodiments wherein the heterologous moiety (e.g., heterologous polypeptide) has a specific function, the heterologous moiety (e.g., heterologous polypeptide) can mediate its function.

In some embodiments, the heterologous moiety is a heterologous polypeptide (e.g., an Ig (e.g., hIg, mIg) Fc region (e.g., an Ig (e.g., hIg, mIg) Fc region)) forming a fusion protein. In some embodiments, the fusion protein comprises from N- to C-terminus: a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)) and a heterologous polypeptide (e.g., an Ig (e.g., hIg, mIg) Fc region (e.g., an Ig (e.g., hIg, mIg) Fc region)). In some embodiments, the fusion protein comprises from N- to C-terminus: a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or the immunogen (e.g., the immunogenic protein (or a functional fragment and/or functional variant thereof) or a nucleic acid molecule encoding the immunogenic protein (or the functional fragment and/or functional variant thereof)), a peptide linker (e.g., described herein), and a heterologous polypeptide (e.g., an Ig (e.g., hIg, mIg) Fc region (e.g., an Ig (e.g., hIg, mIg) Fc region)). In this specific orientation, the N-terminus of the protein described herein (e.g., the hIL-10R binding agent (e.g., the hIL-10R binding protein (or the functional fragment and/or functional variant thereof) or the nucleic acid molecule encoding the hIL-10R binding protein (or the functional fragment and/or functional variant thereof)), the IGIP (e.g., hIGIP) protein (or the functional fragment and/or functional variant thereof) or the nucleic acid molecule encoding the IGIP protein (or the functional fragment and/or functional variant thereof)); and/or the immunogen (e.g., the immunogenic protein (or the functional fragment and/or functional variant thereof) or the nucleic acid molecule encoding the immunogenic protein (or the functional fragment and/or functional variant thereof)) is operably connected to the C-terminus of the heterologous polypeptide (e.g., an Ig (e.g., hIg, mIg) Fc region (e.g., an Ig (e.g., hIg, mIg) Fc region)) either directly or indirectly through the peptide linker (e.g., described herein).

In some embodiments, the fusion polypeptide comprises from N- to C-terminus: a heterologous polypeptide (e.g., an Ig (e.g., hIg, mIg) Fc region (e.g., an Ig (e.g., hIg, mIg) Fc region)) and a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)). In some embodiments, the fusion polypeptide comprises from N- to C-terminus: a heterologous polypeptide (e.g., an Ig (e.g., hIg, mIg) Fc region (e.g., an Ig (e.g., hIg, mIg) Fc region)), a peptide linker (e.g., described herein), and a protein described herein (e.g., a hIL-10R binding agent (e.g., a hIL-10R binding protein (or a functional fragment and/or functional variant thereof) or the nucleic acid molecule encoding the hIL-10R binding protein (or the functional fragment and/or functional variant thereof)), an IGIP (e.g., hIGIP) protein (or a functional fragment and/or functional variant thereof) or a nucleic acid molecule encoding the IGIP protein (or the functional fragment and/or functional variant thereof)); and/or an immunogen (e.g., an immunogenic protein (or a functional fragment and/or functional variant thereof) or a nucleic acid molecule encoding the immunogenic protein (or the functional fragment and/or functional variant thereof)). In this specific orientation, the C-terminus of the protein described herein (e.g., the hIL-10R binding agent (e.g., the hIL-10R binding protein (or the functional fragment and/or functional variant thereof) or the nucleic acid molecule encoding the hIL-10R binding protein (or the functional fragment and/or functional variant thereof)), the IGIP (e.g., hIGIP) protein (or the functional fragment and/or functional variant thereof) or the nucleic acid molecule encoding the IGIP protein (or the functional fragment and/or functional variant thereof)); and/or the immunogen (e.g., the immunogenic protein (or the functional fragment and/or functional variant thereof) or the nucleic acid molecule encoding the immunogenic protein (or the functional fragment and/or functional variant thereof)) is operably connected to the N-terminus of the heterologous polypeptide (e.g., a hIg Fc region (e.g., an Ig (e.g., hIg, mIg) Fc region (e.g., an Ig (e.g., hIg, mIg) Fc region)) either directly or indirectly through the peptide linker (e.g., described herein).

### 5.10.4 Multimeric Fusion Proteins

In one aspect, provided herein are multimeric (e.g., dimeric) proteins comprising at least two protein fusions or conjugates described herein (e.g., Ig (e.g., hIg, mIg) Fc fusion proteins described herein). In some embodiments, the protein is dimeric. In some embodiments, the protein is homodimeric. In some embodiments, the protein is heterodimeric. In some embodiments, the at least two protein fusions (e.g., Ig (e.g., hIg, mIg) Fc fusion proteins) or conjugates associate via covalent or non-covalent interactions. In some embodiments, the at least two protein fusions (e.g., Ig (e.g., hIg, mIg) Fc fusion proteins) or conjugates associate via at least one covalent interaction. In some embodiments, the at least two protein fusions (e.g., Ig (e.g., hIg, mIg) Fc fusion proteins) or conjugates associate via one or more disulfide bond. In some embodiments, the at least two protein fusions (e.g., Ig (e.g., hIg, mIg) Fc fusion proteins) or conjugates associate via 1, 2, 3, 4, or more disulfide bonds.

In some embodiments, the protein is dimeric comprising a first protein fusion (e.g., an Ig (e.g., hIg, mIg) Fc fusion protein) or conjugate described herein and a second protein fusion (e.g., a hIg Fc fusion protein) or conjugate described herein, wherein the first polypeptide comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second polypeptide.

In some embodiments, the protein is dimeric comprising a first Ig (e.g., hIg, mIg) Fc fusion protein and a second Ig (e.g., hIg, mIg) Fc fusion protein. In some embodiments, the dimeric protein is homodimeric. In some embodiments, the dimeric protein is heterodimeric. In some embodiments, the first Ig (e.g., hIg, mIg) Fc fusion protein comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein.

An exemplary dimeric Ig (e.g., hIg, mIg Fc fusion protein includes, for example, a protein comprising (i) a first hIg Fc fusion protein comprising from N- to C-terminus: a first Ig (e.g., hIg, mIg) Fc region (e.g., described herein), a first peptide linker (e.g., described herein), and a first protein described herein (e.g., a hIL-10R binding protein (e.g., described herein), an IGIP protein (e.g., described herein), or an immunogenic protein (e.g., described herein); and (ii) a second Ig (e.g., hIg, mIg) Fc fusion protein comprising from N- to C-terminus: a second Ig (e.g., hIg, mIg) Fc region (e.g., described herein), a second peptide linker (e.g., described herein), and a second protein described herein (e.g., a hIL-10R binding protein (e.g., described herein), an IGIP protein (e.g., described herein), or an immunogenic protein (e.g., described herein). In some embodiments, the amino acid sequence of the first Ig (e.g., hIg, mIg) Fc fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein. In this specific embodiment, the N-terminus of the protein described herein (e.g., a hIL-10R binding protein (e.g., described herein), an IGIP protein (e.g., described herein), or an immunogenic protein (e.g., described herein) is operably connected to the C-terminus of the Ig (e.g., hIg, mIg) Fc region through the peptide linker (e.g., described herein).

Another exemplary dimeric Ig (e.g., hIg, mIg) Fc fusion protein includes, for example, a protein comprising (i) a first Ig (e.g., hIg, mIg) Fc fusion protein comprising from N- to C-terminus: a first protein described herein (e.g., a hIL-10R binding protein (e.g., described herein), an IGIP protein (e.g., described herein), or an immunogenic protein (e.g., described herein), a first peptide linker (e.g., described herein), and a first Ig (e.g., hIg, mIg) Fc region (e.g., described herein); and (ii) a second protein described herein (e.g., a hIL-10R binding protein (e.g., described herein), an IGIP protein (e.g., described herein), or an immunogenic protein (e.g., described herein), a second peptide linker (e.g., described herein), and a second Ig (e.g., hIg, mIg) Fc region (e.g., described herein). In some embodiments, the amino acid sequence of the first Ig (e.g., hIg, mIg) Fc fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein. In this specific embodiment, the C-terminus of the protein described herein (e.g., the hIL-10R binding protein (e.g., described herein), the IGIP protein (e.g., described herein), or the immunogenic protein (e.g., described herein) is operably connected to the N-terminus of the Ig (e.g., hIg, mIg) Fe region either directly or indirectly through the peptide linker (e.g., described herein).

### 5.10.5 Exemplary hIL-10R Binding Protein-Ig Fusion Proteins & Polypeptides

The amino acid sequence of exemplary hIL-10R binding fusion proteins (hIL-10R BFPs) described herein is provided in Table 7. Each of the hIL-10R BFPs 1-2 and 4-14 comprises from N- to C-terminus the hIL-2 signal sequence (hIL-2ss), an effector function reduced hIgG4 Fc region, a peptide linker, and a hIL-10R binding protein identified herein (hIL-10R BPs 1-2 or 4-14) (e.g., see Table 2). Each of the hIL-10R BFP 1-2 or 4-14 comprises from N- to C-terminus an effector function reduced hIgG4 Fc region, a peptide linker, and a hIL-10R binding protein identified herein (hIL-10R BFP 4-14) (e.g., see Table 2, SEQ ID NOS: 4-14). The Fc domain of “m2a” fusion proteins comprises an effector function reduced mIgG2a Fc region. The Fc domain of “m1” fusion proteins comprises an mIgG1 Fc region. The fusion proteins provided in Table 7 are exemplary only, and not intended to be limiting. Similar fusion proteins could made utilizing the additional hIL-10R BPs listed in Table 2, e.g., any one of hIL-10R BPs 3, 15-178.

TABLE 7
The Amino Acid Sequence of Exemplary Ig Fusion Proteins & Polypeptides.

| | | SEQ |
|---|---|---|
| Description | Amino Acid Sequence | ID NO |
| hIL-10R | MYRMQLLSCIALSLALVINSAESKYGPPCPPCPAPEAAGGPSVFLFPPKPKD | 412 |
| BP-1 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMHSSALLCCLVLLTGVRASPGQGTQSENSCTHEPGNLPNMLRDLR | |
| | DAFSRVKTFFQMKDQLDNLLLKESLLEDFKGYLGCQALSEMIQFYLEEVMPQ | |
| | AENQDPDIKAHVNSLGENLKTLRLRLRRCHRFLPCENKSKAVEQVKNAENKL | |
| | QEKGIYKAMSEFDIFINYIEAYMTMKIRN | |
| m2a-hIL- | MYRMQLLSCIALSLALVINSEPRGPTIKPCPPCKCPAPNAAGGPSVFIFPPK | 494 |
| 10R BP-1 | IKDVLMISLSPIVTCVVVDVSEDDPDVQISWFVNNVEVHTAQTQTHREDYNS | |
| with hIL-2 | TLRVVSALPIQHQDWMSGKEFKCKVNNKDLGAPIERTISKPKGSVRAPQVYV | |
| signal | LPPPEEEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGGGGGGG | |
| | SGGGGSGGGGSMHSSALLCCLVLLTGVRASPGQGTQSENSCTHEPGNLPNML | |
| | RDLRDAFSRVKTFFQMKDQLDNLLLKESLLEDFKGYLGCQALSEMIQFYLEE | |
| | VMPQAENQDPDIKAHVNSLGENLKTLRLRLRRCHRFLPCENKSKAVEQVKNA | |
| | FNKLQEKGIYKAMSEFDIFINYIEAYMTMKIRN | |
| m1-hIL-10R | MYRMQLLSCIALSLALVINSVPRDCGCKPCICTVPEVSSVFIFPPKPKDVLT | 495 |
| BP-1 | ITLTPKVTCVVVAISKDDPEVQFSWFVDDVEVHTAQTQPREEQENSTERSVS | |
| with hIL-2 | ELPIMHQDWLNGKEFKCRVNSAAFPAPIEKTISKTKGRPKAPQVYTIPPPKE | |
| signal | QMAKDKVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGGGGSGG | |
| | GGSMHSSALLCCLVLLTGVRASPGQGTQSENSCTHEPGNLPNMLRDLRDAFS | |
| | RVKTFFQMKDQLDNLLLKESLLEDFKGYLGCQALSEMIQFYLEEVMPQAENQ | |
| | DPDIKAHVNSLGENLKTLRLRLRRCHRFLPCENKSKAVEQVKNAFNKLQEKG | |
| | IYKAMSEFDIFINYIEAYMTMKIRN | |
| hIL-10R | MYRMQLLSCIALSLALVTNSAESKYGPPCPPCPAPEAAGGPSVFLFPPKPKD | 414 |
| BP-2 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMHSSALLCCLVLLTGVRASPGQGTQSENSCTHFPGYLPNMLRDLR | |
| | DAFSRVKTFFQMKDQLDNLLLKESLLEDFKGYLGCQALSEMIQFYLEEVMPQ | |
| | AENQDPDIKAHVQSLGENLKDLRLWLRRCHRFLPCENKSKAVEQVKNAENKL | |
| | QEKGIYKAMSEFDIFINYIEAYMTMKIRN | |
| m2a-hIL- | MYRMQLLSCIALSLALVINSEPRGPTIKPCPPCKCPAPNAAGGPSVFIFPPK | 496 |
| 10R BP-2 | IKDVLMISLSPIVTCVVVDVSEDDPDVQISWFVNNVEVHTAQTQTHREDYNS | |
| with hIL-2 | TLRVVSALPIQHQDWMSGKEFKCKVNNKDLGAPIERTISKPKGSVRAPQVYV | |
| signal | LPPPEEEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGGGGGGG | |
| | SGGGGSGGGGSMHSSALLCCLVLLTGVRASPGQGTQSENSCTHFPGYLPNML | |
| | RDLRDAFSRVKTFFQMKDQLDNLLLKESLLEDFKGYLGCQALSEMIQFYLEE | |
| | VMPQAENQDPDIKAHVQSLGENLKDLRLWLRRCHRFLPCENKSKAVEQVKNA | |
| | FNKLQEKGIYKAMSEFDIFINYIEAYMTMKIRN | |
| m1-hIL-10R | MYRMQLLSCIALSLALVTNSVPRDCGCKPCICTVPEVSSVFIFPPKPKDVLT | 497 |
| BP-2 | ITLTPKVTCVVVAISKDDPEVQFSWFVDDVEVHTAQTQPREEQENSTERSVS | |
| with hIL-2 | ELPIMHQDWLNGKEFKCRVNSAAFPAPIEKTISKTKGRPKAPQVYTIPPPKE | |
| signal | QMAKDKVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGGGGSGG | |
| | GGSMHSSALLCCLVLLTGVRASPGQGTQSENSCTHEPGYLPNMLRDLRDAES | |
| | RVKTFFQMKDQLDNLLLKESLLEDFKGYLGCQALSEMIQFYLEEVMPQAENQ | |
| | DPDIKAHVQSLGENLKDLRLWLRRCHRFLPCENKSKAVEQVKNAFNKLQEKG | |
| | IYKAMSEFDIFINYIEAYMTMKIRN | |
| hIL-10R | MYRMQLLSCIALSLALVTNSAESKYGPPCPPCPAPEAAGGPSVFLFPPKPKD | 420 |
| BP-4 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMGKRAFVVSVAMALLGIYVITNTVNARHCMFGDSLRNSPDMKNML | |
| | QDLRGGYSGSGIKRTFQGKDTLDSMLLTQSLLDDFKGYLGCQALSEMIQFYL | |
| | EEVMPQAENHGPTDSVKQLGEKLHTLNQKFGECPRWFPCYYNTTPAVENVKS | |
| | VFSKLQERGVYKAMSEFDIFINYIETYTTMK | |
| hIL-10R | MYRMQLLSCIALSLALVTNSAESKYGPPCPPCPAPEAAGGPSVFLFPPKPKD | 421 |
| BP-5 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMARRLTVASCGSVSLLAAFAAVLLIGCQLESGEALPLGSRSADSR | |
| | SVDGQRVPAPQNNYPGLLRDLRLGYEGFKQKVTDSHPDETLLGSSRLAGDLK | |
| | GPLRCQALSEMIQFLLQVVLPDAENSRQDLRSQFSTLGDRITGLRQQLRRDP | |
| | TVFPCESRSDGVSDLRSAYTRLGSTGAEKVLSEFDIFINYIEAYVTSV | |
| hIL-10R | MYRMQLLSCIALSLALVTNSAESKYGPPCPPCPAPEAAGGPSVFLFPPKPKD | 422 |
| BP-6 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMSNNKILVCAVIILTYTLYTDAYCVEYAESDEDRQQCSSSSNFPA | |
| | SLPHMLRELRAAFGKVKTFFQMKDQLNSMLLTQSLLDDFKGYLGCQALSEMI | |
| | QFYLEEVMPQAENHGPDIKEHVNSLGEKLKTLRLRLRRCHRELPCENKSKAV | |
| | EQVKRVENMLQERGVYKAMSEFDIFINYIESYMTTKM | |
| hIL-10R | MYRMQLLSCIALSLALVTNSAESKYGPPCPPCPAPEAAGGPSVFLFPPKPKD | 423 |
| BP-7 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMANVVYVVLVISIMMANIHVSKTYCTSCSHHQCTEDENQKQDCED | |
| | ANHSLPHMLRELRAAFGKVKTFFQMKDQLHSLLLTQSLLDDEKGYLGCQALS | |
| | EMIQFYLEEVMPQAENHGPEEHDNSLSEHGPDVKEHVNSLGEKLKTLRLRLR | |
| | RCHRFLPCENKSKAVEKVKRVFSELQERGVYKAMSEFDIFINYIETYMTT | |
| hIL-10R | MYRMQLLSCIALSLALVTNSAESKYGPPCPPCPAPEAAGGPSVFLFPPKPKD | 424 |
| BP-8 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMQGLQLLRGLLCCGVFAAASSRSPKNKPSIDCNPQTGDFVNMLKS | |
| | MRQDYSRIRDTLHDRDKLHSSLLTGALLDEMMGYSGCRTTLLLMEHYLDTWY | |
| | PAAYRHHLYDNQTLVVVDRMGSTLVALLKAMVQCPMLACGAPSPAMDKMLQQ | |
| | EAKMKKYTGVYKGISETDLLLGYLELYMMKEKR | |
| hIL-10R | MYRMQLLSCIALSLALVTNSAESKYGPPCPPCPAPEAAGGPSVFLFPPKPKD | 425 |
| BP-9 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMRRRRSFGIVVSGAIRTLLMVAVVAVSVRGHEHKVPPACDPVHGN | |
| | LAGIFKELRAIYASIREALQKKDTVYYTSLFNDRVLQEMLSPMGCRVTNELM | |
| | EHYLDGVLPRAAHEDYDNSTLNGLHAFTSSMQALYQHMLKCPALACTGKTPA | |
| | WMYFLEVEHKLNPWRGTAKAAAEADLLLNYLETELLQF | |
| hIL-10R | MYRMQLLSCIALSLALVTNSAESKYGPPCPPCPAPEAAGGPSVFLFPPKPKD | 416 |
| BP-10 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMGSRRLSRCSFATAVCLVAIVAAVAAKGRDSKPSPACDPMHGALA | |
| | GIFKELRTTYRSVREALQTKDTVYYVSLFHEQLLQEMLSPVGCRVTNELMQH | |
| | YLDGVLPRAFHCGYDNATLNALHALSSSLSTLYQHMLKCPALACTGQTPAWT | |
| | QFLDTEHKLDPWKGTVKATAEMDLLLNYLETELLQS | |
| m2a-hIL- | MYRMQLLSCIALSLALVTNSEPRGPTIKPCPPCKCPAPNAAGGPSVFIFPPK | 498 |
| 10R BP-10 | IKDVLMISLSPIVTCVVVDVSEDDPDVQISWFVNNVEVHTAQTQTHREDYNS | |
| with hIL-2 | TLRVVSALPIQHQDWMSGKEFKCKVNNKDLGAPIERTISKPKGSVRAPQVYV | |
| signal | LPPPEEEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGGGGGGG | |
| | GSGGGGSGGGGSMGSRRLSRCSFATAVCLVAIVAAVAAKGRDSKPSPACDPM | |
| | HGALAGIFKELRTTYRSVREALQTKDTVYYVSLFHEQLLQEMLSPVGCRVTN | |
| | ELMQHYLDGVLPRAFHCGYDNATLNALHALSSSLSTLYQHMLKCPALACTGQ | |
| | TPAWTQFLDTEHKLDPWKGTVKATAEMDLLLNYLETELLQS | |
| m1-hIL-10R | MYRMQLLSCIALSLALVTNSVPRDCGCKPCICTVPEVSSVFIFPPKPKDVLT | 499 |
| BP-10 | ITLTPKVTCVVVAISKDDPEVQFSWFVDDVEVHTAQTQPREEQENSTERSVS | |
| with hIL-2 | ELPIMHQDWLNGKEFKCRVNSAAFPAPIEKTISKTKGRPKAPQVYTIPPPKE | |
| signal | QMAKDKVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGGGGSGG | |
| | GGSMGSRRLSRCSFATAVCLVAIVAAVAAKGRDSKPSPACDPMHGALAGIFK | |
| | ELRTTYRSVREALQTKDTVYYVSLFHEQLLQEMLSPVGCRVTNELMQHYLDG | |
| | VLPRAFHCGYDNATLNALHALSSSLSTLYQHMLKCPALACTGQTPAWTQELD | |
| | TEHKLDPWKGTVKATAEMDLLLNYLETELLQS | |
| hIL-10R | MYRMQLLSCIALSLALVTNSAESKYGPPCPPCPAPEAAGGPSVFLFPPKPKD | 426 |
| BP-11 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMLSVMVSSSLVLIVFFLGASEEAKPATTTIKNTKPQCRPEDYATR | |
| | LQDLRVTFHRVKPTLQREDDYSVWLDGTVVKGCWGCSVMDWLLRRYLEIVEP | |
| | AGDHVYPGLKTELHSMRSTLESIYKDMRQCPLLGCGDKSVISRLSQEAERKS | |
| | DNGTRKGLSELDTLFSRLEEYLHSRK | |
| hIL-10R | MYRMQLLSCIALSLALVTNSAESKYGPPCPPCPAPEAAGGPSVFLFPPKPKD | 427 |
| BP-12 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMALAHQLPVWIESIWILYFTLPLSEERVLPLRGNCKLLLQDTVIP | |
| | NLLYSMRSIFQDIKPYFQGKDSLNNLLLSGQLLEDLQSPIGCDALSEMIQFY | |
| | LEEVMPQAEIHHPKHKNSVMQLGETLHTLISQLQECTALFPCKHKSLGAQKI | |
| | KEEVSKLGQYGIIKAVAEFDIFINYMESYFGVK | |
| hIL-10R | MYRMQLLSCIALSLALVINSAESKYGPPCPPCPAPEAAGGPSVFLFPPKPKD | 428 |
| BP-13 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSMRRRRRSFGIIVAGAIGTLLMMAVVVLSAHDHEHKEVPPACDPVH | |
| | GNLAGIFKELRATYASIREGLQKKDTVYYTSLENDRVLHEMLSPMGCRVTNE | |
| | LMEHYLDGVLPRASHLDYDNSTLNGLHVFASSMQALYQHMLKCPALACTGKT | |
| | PAWMYFLEVEHKLNPWRGTAKAAAEADLLLNYLETELLQF | |
| hIL-10R | MYRMQLLSCIALSLALVTNSAESKYGPPCPPCPAPEAAGGPSVFLFPPKPKD | 429 |
| BP-14 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSMRRRRSFGIVVAGAIGTLLMMAVVVESAHEHKEVPPACDPVHGNL | |
| | AGIFKELRATYASIREGLQKKDTVYYTSLENDRVLQEMLSPMGCRVTNELME | |
| | HYLDGVLPRALHLDYDNSTLNGLHAFASSMQALYQHMLKCPALACTGKTPAW | |
| | MYFLEVEHKLNPWRGTAKAAAEADLLLNYLETELLQF | |
| hIL-10R | AESKYGPPCPPCPAPEAAGGPSVFLFPPKPKDTLMISRTPEVTCVVVDVSQE | 413 |
| BP-1 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMHSSALLCCLVLL | |
| | TGVRASPGQGTQSENSCTHEPGNLPNMLRDLRDAFSRVKTFFQMKDQLDNLL | |
| | LKESLLEDFKGYLGCQALSEMIQFYLEEVMPQAENQDPDIKAHVNSLGENLK | |
| | TLRLRLRRCHRFLPCENKSKAVEQVKNAFNKLQEKGIYKAMSEFDIFINYIE | |
| | AYMTMKIRN | |
| m2a-hIL- | EPRGPTIKPCPPCKCPAPNAAGGPSVFIFPPKIKDVLMISLSPIVTCVVVDV | 500 |
| 10R BP-1 | SEDDPDVQISWFVNNVEVHTAQTQTHREDYNSTLRVVSALPIQHQDWMSGKE | |
| without hIL- | FKCKVNNKDLGAPIERTISKPKGSVRAPQVYVLPPPEEEMTKKQVTLTCMVT | |
| 2 signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGSGGGGSGGGGSMHSSALLCC | |
| | LVLLTGVRASPGQGTQSENSCTHEPGNLPNMLRDLRDAFSRVKTFFQMKDQL | |
| | DNLLLKESLLEDFKGYLGCQALSEMIQFYLEEVMPQAENQDPDIKAHVNSLG | |
| | ENLKTLRLRLRRCHRFLPCENKSKAVEQVKNAFNKLQEKGIYKAMSEFDIFI | |
| | NYIEAYMTMKIRN | |
| m1-hIL-10R | VPRDCGCKPCICTVPEVSSVFIFPPKPKDVLTITLTPKVTCVVVAISKDDPE | 501 |
| BP-1 | VQFSWFVDDVEVHTAQTQPREEQENSTERSVSELPIMHQDWLNGKEFKCRVN | |
| without hIL- | SAAFPAPIEKTISKTKGRPKAPQVYTIPPPKEQMAKDKVSLTCMITDFFPED | |
| 2 signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGSGGGGSMHSSALLCCLVLLTGVR | |
| | ASPGQGTQSENSCTHFPGNLPNMLRDLRDAFSRVKTFFQMKDQLDNLLLKES | |
| | LLEDFKGYLGCQALSEMIQFYLEEVMPQAENQDPDIKAHVNSLGENLKTLRL | |
| | RLRRCHRFLPCENKSKAVEQVKNAFNKLQEKGIYKAMSEFDIFINYIEAYMT | |
| | MKIRN | |
| hIL-10R | AESKYGPPCPPCPAPEAAGGPSVFLFPPKPKDTLMISRTPEVTCVVVDVSQE | 415 |
| BP-2 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMHSSALLCCLVLL | |
| | TGVRASPGQGTQSENSCTHFPGYLPNMLRDLRDAFSRVKTFFQMKDQLDNLL | |
| | LKESLLEDFKGYLGCQALSEMIQFYLEEVMPQAENQDPDIKAHVQSLGENLK | |
| | DLRLWLRRCHRFLPCENKSKAVEQVKNAFNKLQEKGIYKAMSEFDIFINYIE | |
| | AYMTMKIRN | |
| m2a-hIL- | EPRGPTIKPCPPCKCPAPNAAGGPSVFIFPPKIKDVLMISLSPIVTCVVVDV | 502 |
| 10R BP-2 | SEDDPDVQISWFVNNVEVHTAQTQTHREDYNSTLRVVSALPIQHQDWMSGKE | |
| without hIL- | FKCKVNNKDLGAPIERTISKPKGSVRAPQVYVLPPPEEEMTKKQVTLTCMVT | |
| 2 signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGSGGGGSGGGGSMHSSALLCC | |
| | LVLLTGVRASPGQGTQSENSCTHFPGYLPNMLRDLRDAFSRVKTFFQMKDQL | |
| | DNLLLKESLLEDFKGYLGCQALSEMIQFYLEEVMPQAENQDPDIKAHVQSLG | |
| | ENLKDLRLWLRRCHRFLPCENKSKAVEQVKNAFNKLQEKGIYKAMSEFDIFI | |
| | NYIEAYMTMKIRN | |
| m1-hIL-10R | VPRDCGCKPCICTVPEVSSVFIFPPKPKDVLTITLTPKVTCVVVAISKDDPE | 503 |
| BP-2 | VQFSWFVDDVEVHTAQTQPREEQFNSTFRSVSELPIMHQDWLNGKEFKCRVN | |
| without hIL- | SAAFPAPIEKTISKTKGRPKAPQVYTIPPPKEQMAKDKVSLTCMITDFFPED | |
| 2 signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGSGGGGSMHSSALLCCLVLLTGVR | |
| | ASPGQGTQSENSCTHFPGYLPNMLRDLRDAFSRVKTFFQMKDQLDNLLLKES | |
| | LLEDFKGYLGCQALSEMIQFYLEEVMPQAENQDPDIKAHVQSLGENLKDLRL | |
| | WLRRCHRFLPCENKSKAVEQVKNAFNKLQEKGIYKAMSEFDIFINYIEAYMT | |
| | MKIRN | |
| hIL-10R | AESKYGPPCPPCPAPEAAGGPSVFLFPPKPKDTLMISRTPEVTCVVVDVSQE | 430 |
| BP-4 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMGKRAFVVSVAMA | |
| | LLGIYVITNTVNARHCMFGDSLRNSPDMKNMLQDLRGGYSGSGIKRTFQGKD | |
| | TLDSMLLTQSLLDDEKGYLGCQALSEMIQFYLEEVMPQAENHGPTDSVKQLG | |
| | EKLHTLNQKFGECPRWFPCYYNTTPAVENVKSVESKLQERGVYKAMSEFDIE | |
| | INYIETYTTMK | |
| hIL-10R | AESKYGPPCPPCPAPEAAGGPSVFLFPPKPKDTLMISRTPEVTCVVVDVSQE | 431 |
| BP-5 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMARRLTVASCGSV | |
| | SLLAAFAAVLLIGCQLESGEALPLGSRSADSRSVDGQRVPAPQNNYPGLLRD | |
| | LRLGYEGFKQKVTDSHPDETLLGSSRLAGDLKGPLRCQALSEMIQFLLQVVL | |
| | PDAENSRQDLRSQFSTLGDRITGLRQQLRRDPTVFPCESRSDGVSDLRSAYT | |
| | RLGSTGAEKVLSEFDIFINYIEAYVTSV | |
| hIL-10R | AESKYGPPCPPCPAPEAAGGPSVFLFPPKPKDTLMISRTPEVTCVVVDVSQE | 432 |
| BP-6 | DPEVQFNWYVDGVEVHNAKTKPREEQFNSTYRVVSVLTVLHQDWINGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMSNNKILVCAVII | |
| | LTYTLYTDAYCVEYAESDEDRQQCSSSSNEPASLPHMLRELRAAFGKVKTFF | |
| | QMKDQLNSMLLTQSLLDDFKGYLGCQALSEMIQFYLEEVMPQAENHGPDIKE | |
| | HVNSLGEKLKTLRLRLRRCHRFLPCENKSKAVEQVKRVENMLQERGVYKAMS | |
| | EFDIFINYIESYMTTKM | |
| hIL-10R | AESKYGPPCPPCPAPEAAGGPSVFLFPPKPKDTLMISRTPEVTCVVVDVSQE | 433 |
| BP-7 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVFSC | |
| peptide | SVMHEALHNHYTQKSLSLSLGGGGGGGGSGGGGSGGGGSMANVVYVVLVISI | |
| | MMANIHVSKTYCTSCSHHQCTEDENQKQDCEDANHSLPHMLRELRAAFGKVK | |
| | TFFQMKDQLHSLLLTQSLLDDEKGYLGCQALSEMIQFYLEEVMPQAENHGPE | |
| | EHDNSLSEHGPDVKEHVNSLGEKLKTLRLRLRRCHRFLPCENKSKAVEKVKR | |
| | VESELQERGVYKAMSEFDIFINYIETYMTT | |
| hIL-10R | AESKYGPPCPPCPAPEAAGGPSVFLFPPKPKDTLMISRTPEVTCVVVDVSQE | 434 |
| BP-8 | DPEVQFNWYVDGVEVHNAKTKPREEQFNSTYRVVSVLTVLHQDWLNGKEYKC | |
| with hIL-2 | KVSNKGLPSSIEKTISKAKGQPREPQVYTLPPSQEEMTKNQVSLTCLVKGFY | |
| signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMQGLQLLRGLLCC | |
| | GVFAAASSRSPKNKPSIDCNPQTGDFVNMLKSMRQDYSRIRDTLHDRDKLHS | |
| | SLLTGALLDEMMGYSGCRTTLLLMEHYLDTWYPAAYRHHLYDNQTLVVVDRM | |
| | GSTLVALLKAMVQCPMLACGAPSPAMDKMLQQEAKMKKYTGVYKGISETDLL | |
| | LGYLELYMMKEKR | |
| hIL-10R | AESKYGPPCPPCPAPEAAGGPSVFLFPPKPKDTLMISRTPEVTCVVVDVSQE | 435 |
| BP-9 | DPEVQFNWYVDGVEVHNAKTKPREEQFNSTYRVVSVLTVLHQDWINGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMRRRRSFGIVVSG | |
| | AIRTLLMVAVVAVSVRGHEHKVPPACDPVHGNLAGIFKELRAIYASIREALQ | |
| | KKDTVYYTSLENDRVLQEMLSPMGCRVTNELMEHYLDGVLPRAAHEDYDNST | |
| | LNGLHAFTSSMQALYQHMLKCPALACTGKTPAWMYFLEVEHKLNPWRGTAKA | |
| | AAEADLLLNYLETFLLQF | |
| hIL-10R | AESKYGPPCPPCPAPEAAGGPSVFLFPPKPKDTLMISRTPEVTCVVVDVSQE | 417 |
| BP-10 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMGSRRLSRCSFAT | |
| | AVCLVAIVAAVAAKGRDSKPSPACDPMHGALAGIFKELRTTYRSVREALQTK | |
| | DTVYYVSLFHEQLLQEMLSPVGCRVTNELMQHYLDGVLPRAFHCGYDNATLN | |
| | ALHALSSSLSTLYQHMLKCPALACTGQTPAWTQFLDTEHKLDPWKGTVKATA | |
| | EMDLLLNYLETELLQS | |
| m2a-hIL- | EPRGPTIKPCPPCKCPAPNAAGGPSVFIFPPKIKDVLMISLSPIVTCVVVDV | 504 |
| 10R BP-10 | SEDDPDVQISWFVNNVEVHTAQTQTHREDYNSTLRVVSALPIQHQDWMSGKE | |
| without hIL- | FKCKVNNKDLGAPIERTISKPKGSVRAPQVYVLPPPEEEMTKKQVTLTCMVT | |
| 2 signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGGSGGGGSGGGGSMGSRRLSR | |
| | CSFATAVCLVAIVAAVAAKGRDSKPSPACDPMHGALAGIFKELRTTYRSVRE | |
| | ALQTKDTVYYVSLFHEQLLQEMLSPVGCRVTNELMQHYLDGVLPRAFHCGYD | |
| | NATLNALHALSSSLSTLYQHMLKCPALACTGQTPAWTQFLDTEHKLDPWKGT | |
| | VKATAEMDLLLNYLETELLQS | |
| m1-hIL-10R | VPRDCGCKPCICTVPEVSSVFIFPPKPKDVLTITLTPKVTCVVVAISKDDPE | 505 |
| BP-10 | VQFSWFVDDVEVHTAQTQPREEQFNSTFRSVSELPIMHQDWLNGKEFKCRVN | |
| without hIL- | SAAFPAPIEKTISKTKGRPKAPQVYTIPPPKEQMAKDKVSLTCMITDFFPED | |
| 2 signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGSGGGGSMGSRRLSRCSFATAVCL | |
| | VAIVAAVAAKGRDSKPSPACDPMHGALAGIFKELRTTYRSVREALQTKDTVY | |
| | YVSLFHEQLLQEMLSPVGCRVTNELMQHYLDGVLPRAFHCGYDNATLNALHA | |
| | LSSSLSTLYQHMLKCPALACTGQTPAWTQFLDTEHKLDPWKGTVKATAEMDL | |
| | LLNYLETELLQS | |
| hIL-10R | AESKYGPPCPPCPAPEAAGGPSVFLFPPKPKDTLMISRTPEVTCVVVDVSQE | 436 |
| BP-11 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMLSVMVSSSLVLI | |
| | VFFLGASEEAKPATTTIKNTKPQCRPEDYATRLQDLRVTFHRVKPTLQREDD | |
| | YSVWLDGTVVKGCWGCSVMDWLLRRYLEIVFPAGDHVYPGLKTELHSMRSTL | |
| | ESIYKDMRQCPLLGCGDKSVISRLSQEAERKSDNGTRKGLSELDTLESRLEE | |
| | YLHSRK | |
| hIL-10R | AESKYGPPCPPCPAPEAAGGPSVFLFPPKPKDTLMISRTPEVTCVVVDVSQE | 437 |
| BP-12 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMALAHQLPVWIES | |
| | IWILYFTLPLSEERVLPLRGNCKLLLQDTVIPNLLYSMRSIFQDIKPYFQGK | |
| | DSLNNLLLSGQLLEDLQSPIGCDALSEMIQFYLEEVMPQAEIHHPKHKNSVM | |
| | QLGETLHTLISQLQECTALFPCKHKSLGAQKIKEEVSKLGQYGIIKAVAEFD | |
| | IFINYMESYFGVK | |
| hIL-10R | AESKYGPPCPPCPAPEAAGGPSVFLFPPKPKDTLMISRTPEVTCVVVDVSQE | 438 |
| BP-13 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSMRRRRRSEGIIVA | |
| | GAIGTLLMMAVVVLSAHDHEHKEVPPACDPVHGNLAGIFKELRATYASIREG | |
| | LQKKDTVYYTSLENDRVLHEMLSPMGCRVTNELMEHYLDGVLPRASHLDYDN | |
| | STLNGLHVFASSMQALYQHMLKCPALACTGKTPAWMYFLEVEHKLNPWRGTA | |
| | KAAAEADLLLNYLETFLLQF | |
| hIL-10R | AESKYGPPCPPCPAPEAAGGPSVFLFPPKPKDTLMISRTPEVTCVVVDVSQE | 439 |
| BP-14 | DPEVQFNWYVDGVEVHNAKTKPREEQENSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVFSC | |
| peptide | SVMHEALHNHYTQKSLSLSLGGGGGGGGSGGGGSGGGGSMRRRRSFGIVVAG | |
| | AIGTLLMMAVVVESAHEHKEVPPACDPVHGNLAGIFKELRATYASIREGLQK | |
| | KDTVYYTSLFNDRVLQEMLSPMGCRVTNELMEHYLDGVLPRALHLDYDNSTL | |
| | NGLHAFASSMQALYQHMLKCPALACTGKTPAWMYFLEVEHKLNPWRGTAKAA | |
| | AEADLLLNYLETFLLQF | |
| hIL-10R | MYRMQLLSCIALSLALVTNSESKYGPPCPPCPAPEAAGGPSVFLFPPKPKDT | 440 |
| BP-1 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFFL | |
| peptide | YSRLTVDKSRWQEGNVFSCSVMHEALHNHYTQKSLSLSLGGGGGGGGSGGGG | |
| | SGGGGSMHSSALLCCLVLLTGVRASPGQGTQSENSCTHEPGNLPNMLRDLRD | |
| | AFSRVKTFFQMKDQLDNLLLKESLLEDFKGYLGCQALSEMIQFYLEEVMPQA | |
| | ENQDPDIKAHVNSLGENLKTLRLRLRRCHRFLPCENKSKAVEQVKNAENKLQ | |
| | EKGIYKAMSEFDIFINYIEAYMTMKIRN | |
| hIL-10R | MYRMQLLSCIALSLALVTNSESKYGPPCPPCPAPEAAGGPSVFLFPPKPKDT | 441 |
| BP-2 | LMISRTPEVTCVVVDVSQEDPEVQFNWYVDGVEVHNAKTKPREEQFNSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMHSSALLCCLVLLTGVRASPGQGTQSENSCTHFPGYLPNMLRDLRD | |
| | AFSRVKTFFQMKDQLDNLLLKESLLEDFKGYLGCQALSEMIQFYLEEVMPQA | |
| | ENQDPDIKAHVQSLGENLKDLRLWLRRCHRFLPCENKSKAVEQVKNAFNKLQ | |
| | EKGIYKAMSEFDIFINYIEAYMTMKIRN | |
| hIL-10R | MYRMQLLSCIALSLALVTNSESKYGPPCPPCPAPEAAGGPSVFLFPPKPKDT | 442 |
| BP-4 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVESCSVMHEALHNHYTQKSLSLSLGGGGGGGGSGGGG | |
| | SGGGGSMGKRAFVVSVAMALLGIYVITNTVNARHCMFGDSLRNSPDMKNMLQ | |
| | DLRGGYSGSGIKRTFQGKDTLDSMLLTQSLLDDFKGYLGCQALSEMIQFYLE | |
| | EVMPQAENHGPTDSVKQLGEKLHTLNQKFGECPRWFPCYYNTTPAVENVKSV | |
| | FSKLQERGVYKAMSEFDIFINYIETYTTMK | |
| hIL-10R | MYRMQLLSCIALSLALVINSESKYGPPCPPCPAPEAAGGPSVFLFPPKPKDT | 443 |
| BP-5 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTIPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMARRLTVASCGSVSLLAAFAAVLLIGCQLESGEALPLGSRSADSRS | |
| | VDGQRVPAPQNNYPGLLRDLRLGYEGFKQKVTDSHPDETLLGSSRLAGDLKG | |
| | PLRCQALSEMIQFLLQVVLPDAENSRQDLRSQFSTLGDRITGLRQQLRRDPT | |
| | VFPCESRSDGVSDLRSAYTRLGSTGAEKVLSEFDIFINYIEAYVTSV | |
| hIL-10R | MYRMQLLSCIALSLALVTNSESKYGPPCPPCPAPEAAGGPSVFLFPPKPKDT | 444 |
| BP-6 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMSNNKILVCAVIILTYTLYTDAYCVEYAESDEDRQQCSSSSNEPAS | |
| | LPHMLRELRAAFGKVKTFFQMKDQLNSMLLTQSLLDDEKGYLGCQALSEMIQ | |
| | FYLEEVMPQAENHGPDIKEHVNSLGEKLKTLRLRLRRCHRFLPCENKSKAVE | |
| | QVKRVENMLQERGVYKAMSEFDIFINYIESYMTTKM | |
| hIL-10R | MYRMQLLSCIALSLALVTNSESKYGPPCPPCPAPEAAGGPSVFLFPPKPKDT | 445 |
| BP-7 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMANVVYVVLVISIMMANIHVSKTYCTSCSHHQCTEDENQKQDCEDA | |
| | NHSLPHMLRELRAAFGKVKTFFQMKDQLHSLLLTQSLLDDEKGYLGCQALSE | |
| | MIQFYLEEVMPQAENHGPEEHDNSLSEHGPDVKEHVNSLGEKLKTLRLRLRR | |
| | CHRFLPCENKSKAVEKVKRVESELQERGVYKAMSEFDIFINYIETYMTT | |
| hIL-10R | MYRMQLLSCIALSLALVTNSESKYGPPCPPCPAPEAAGGPSVFLFPPKPKDT | 446 |
| BP-8 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFFL | |
| peptide | YSRLTVDKSRWQEGNVFSCSVMHEALHNHYTQKSLSLSLGGGGGGGGSGGGG | |
| | SGGGGSMQGLQLLRGLLCCGVFAAASSRSPKNKPSIDCNPQTGDFVNMLKSM | |
| | RQDYSRIRDTLHDRDKLHSSLLTGALLDEMMGYSGCRTTLLLMEHYLDTWYP | |
| | AAYRHHLYDNQTLVVVDRMGSTLVALLKAMVQCPMLACGAPSPAMDKMLQQE | |
| | AKMKKYTGVYKGISETDLLLGYLELYMMKEKR | |
| hIL-10R | MYRMQLLSCIALSLALVTNSESKYGPPCPPCPAPEAAGGPSVFLFPPKPKDT | 447 |
| BP-9 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMRRRRSFGIVVSGAIRTLLMVAVVAVSVRGHEHKVPPACDPVHGNL | |
| | AGIFKELRAIYASIREALQKKDTVYYTSLENDRVLQEMLSPMGCRVTNELME | |
| | HYLDGVLPRAAHFDYDNSTLNGLHAFTSSMQALYQHMLKCPALACTGKTPAW | |
| | MYFLEVEHKLNPWRGTAKAAAEADLLLNYLETELLQF | |
| hIL-10R | MYRMQLLSCIALSLALVTNSESKYGPPCPPCPAPEAAGGPSVFLFPPKPKDT | 448 |
| BP-10 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVESCSVMHEALHNHYTQKSLSLSLGGGGGGGGSGGGG | |
| | SGGGGSMGSRRLSRCSFATAVCLVAIVAAVAAKGRDSKPSPACDPMHGALAG | |
| | IFKELRTTYRSVREALQTKDTVYYVSLFHEQLLQEMLSPVGCRVTNELMQHY | |
| | LDGVLPRAFHCGYDNATLNALHALSSSLSTLYQHMLKCPALACTGQTPAWTQ | |
| | FLDTEHKLDPWKGTVKATAEMDLLLNYLETFLLQS | |
| hIL-10R | MYRMQLLSCIALSLALVTNSESKYGPPCPPCPAPEAAGGPSVFLFPPKPKDT | 449 |
| BP-11 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVESCSVMHEALHNHYTQKSLSLSLGGGGGGGGSGGGG | |
| | SGGGGSMLSVMVSSSLVLIVFFLGASEEAKPATTTIKNTKPQCRPEDYATRL | |
| | QDLRVTFHRVKPTLQREDDYSVWLDGTVVKGCWGCSVMDWLLRRYLEIVEPA | |
| | GDHVYPGLKTELHSMRSTLESIYKDMRQCPLLGCGDKSVISRLSQEAERKSD | |
| | NGTRKGLSELDTLFSRLEEYLHSRK | |
| hIL-10R | MYRMQLLSCIALSLALVINSESKYGPPCPPCPAPEAAGGPSVFLFPPKPKDT | 450 |
| BP-12 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMALAHQLPVWIFSIWILYFTLPLSEERVLPLRGNCKLLLQDTVIPN | |
| | LLYSMRSIFQDIKPYFQGKDSLNNLLLSGQLLEDLQSPIGCDALSEMIQFYL | |
| | EEVMPQAEIHHPKHKNSVMQLGETLHTLISQLQECTALFPCKHKSLGAQKIK | |
| | EEVSKLGQYGIIKAVAEFDIFINYMESYFGVK | |
| hIL-10R | MYRMQLLSCIALSLALVTNSESKYGPPCPPCPAPEAAGGPSVFLFPPKPKDT | 451 |
| BP-13 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMRRRRRSFGIIVAGAIGTLLMMAVVVLSAHDHEHKEVPPACDPVHG | |
| | NLAGIFKELRATYASIREGLQKKDTVYYTSLENDRVLHEMLSPMGCRVTNEL | |
| | MEHYLDGVLPRASHLDYDNSTLNGLHVFASSMQALYQHMLKCPALACTGKTP | |
| | AWMYFLEVEHKLNPWRGTAKAAAEADLLLNYLETFLLQF | |
| hIL-10R | MYRMQLLSCIALSLALVTNSESKYGPPCPPCPAPEAAGGPSVFLFPPKPKDT | 452 |
| BP-14 | LMISRTPEVTCVVVDVSQEDPEVQFNWYVDGVEVHNAKTKPREEQFNSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSMRRRRSFGIVVAGAIGTLLMMAVVVFSAHEHKEVPPACDPVHGNLA | |
| | GIFKELRATYASIREGLQKKDTVYYTSLENDRVLQEMLSPMGCRVTNELMEH | |
| | YLDGVLPRALHLDYDNSTLNGLHAFASSMQALYQHMLKCPALACTGKTPAWM | |
| | YFLEVEHKLNPWRGTAKAAAEADLLLNYLETELLQF | |
| hIL-10R | ESKYGPPCPPCPAPEAAGGPSVFLFPPKPKDTLMISRTPEVTCVVVDVSQED | 453 |
| BP-1 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMHSSALLCCLVLLT | |
| | GVRASPGQGTQSENSCTHFPGNLPNMLRDLRDAFSRVKTFFQMKDQLDNLLL | |
| | KESLLEDFKGYLGCQALSEMIQFYLEEVMPQAENQDPDIKAHVNSLGENLKT | |
| | LRLRLRRCHRFLPCENKSKAVEQVKNAFNKLQEKGIYKAMSEFDIFINYIEA | |
| | YMTMKIRN | |
| hIL-10R | ESKYGPPCPPCPAPEAAGGPSVFLFPPKPKDTLMISRTPEVTCVVVDVSQED | 454 |
| BP-2 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMHSSALLCCLVLLT | |
| | GVRASPGQGTQSENSCTHFPGYLPNMLRDLRDAFSRVKTFFQMKDQLDNLLL | |
| | KESLLEDFKGYLGCQALSEMIQFYLEEVMPQAENQDPDIKAHVQSLGENLKD | |
| | LRLWLRRCHRFLPCENKSKAVEQVKNAFNKLQEKGIYKAMSEFDIFINYIEA | |
| | YMTMKIRN | |
| hIL-10R | ESKYGPPCPPCPAPEAAGGPSVFLFPPKPKDTLMISRTPEVTCVVVDVSQED | 455 |
| BP-4 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMGKRAFVVSVAMAL | |
| | LGIYVITNTVNARHCMFGDSLRNSPDMKNMLQDLRGGYSGSGIKRTFQGKDT | |
| | LDSMLLTQSLLDDFKGYLGCQALSEMIQFYLEEVMPQAENHGPTDSVKQLGE | |
| | KLHTLNQKFGECPRWFPCYYNTTPAVENVKSVESKLQERGVYKAMSEFDIFI | |
| | NYIETYTTMK | |
| hIL-10R | ESKYGPPCPPCPAPEAAGGPSVFLFPPKPKDTLMISRTPEVTCVVVDVSQED | 456 |
| BP-5 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMARRLTVASCGSVS | |
| | LLAAFAAVLLIGCQLESGEALPLGSRSADSRSVDGQRVPAPQNNYPGLLRDL | |
| | RLGYEGFKQKVTDSHPDETLLGSSRLAGDLKGPLRCQALSEMIQFLLQVVLP | |
| | DAENSRQDLRSQFSTLGDRITGLRQQLRRDPTVFPCESRSDGVSDLRSAYTR | |
| | LGSTGAEKVLSEFDIFINYIEAYVTSV | |
| hIL-10R | ESKYGPPCPPCPAPEAAGGPSVFLFPPKPKDTLMISRTPEVTCVVVDVSQED | 457 |
| BP-6 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMSNNKILVCAVIIL | |
| | TYTLYTDAYCVEYAESDEDRQQCSSSSNFPASLPHMLRELRAAFGKVKTFFQ | |
| | MKDQLNSMLLTQSLLDDFKGYLGCQALSEMIQFYLEEVMPQAENHGPDIKEH | |
| | VNSLGEKLKTLRLRLRRCHRFLPCENKSKAVEQVKRVENMLQERGVYKAMSE | |
| | FDIFINYIESYMTTKM | |
| hIL-10R | ESKYGPPCPPCPAPEAAGGPSVFLFPPKPKDTLMISRTPEVTCVVVDVSQED | 458 |
| BP-7 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMANVVYVVLVISIM | |
| | MANIHVSKTYCTSCSHHQCTEDENQKQDCEDANHSLPHMLRELRAAFGKVKT | |
| | FFQMKDQLHSLLLTQSLLDDFKGYLGCQALSEMIQFYLEEVMPQAENHGPEE | |
| | HDNSLSEHGPDVKEHVNSLGEKLKTLRLRLRRCHRFLPCENKSKAVEKVKRV | |
| | FSELQERGVYKAMSEFDIFINYIETYMTT | |
| hIL-10R | ESKYGPPCPPCPAPEAAGGPSVFLFPPKPKDTLMISRTPEVTCVVVDVSQED | 459 |
| BP-8 | PEVQFNWYVDGVEVHNAKTKPREEQFNSTYRVVSVLTVLHQDWLNGKEYKCK | |
| with hIL-2 | VSNKGLPSSIEKTISKAKGQPREPQVYTLPPSQEEMTKNQVSLTCLVKGFYP | |
| signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMQGLQLLRGLLCCG | |
| | VFAAASSRSPKNKPSIDCNPQTGDFVNMLKSMRQDYSRIRDTLHDRDKLHSS | |
| | LLTGALLDEMMGYSGCRTTLLLMEHYLDTWYPAAYRHHLYDNQTLVVVDRMG | |
| | STLVALLKAMVQCPMLACGAPSPAMDKMLQQEAKMKKYTGVYKGISETDLLL | |
| | GYLELYMMKFKR | |
| hIL-10R | ESKYGPPCPPCPAPEAAGGPSVFLFPPKPKDTLMISRTPEVTCVVVDVSQED | 460 |
| BP-9 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMRRRRSFGIVVSGA | |
| | IRTLLMVAVVAVSVRGHEHKVPPACDPVHGNLAGIFKELRAIYASIREALQK | |
| | KDTVYYTSLFNDRVLQEMLSPMGCRVTNELMEHYLDGVLPRAAHEDYDNSTL | |
| | NGLHAFTSSMQALYQHMLKCPALACTGKTPAWMYFLEVEHKLNPWRGTAKAA | |
| | AEADLLLNYLETELLQF | |
| hIL-10R | ESKYGPPCPPCPAPEAAGGPSVFLFPPKPKDTLMISRTPEVTCVVVDVSQED | 461 |
| BP-10 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMGSRRLSRCSFATA | |
| | VCLVAIVAAVAAKGRDSKPSPACDPMHGALAGIFKELRTTYRSVREALQTKD | |
| | TVYYVSLFHEQLLQEMLSPVGCRVTNELMQHYLDGVLPRAFHCGYDNATLNA | |
| | LHALSSSLSTLYQHMLKCPALACTGQTPAWTQFLDTEHKLDPWKGTVKATAE | |
| | MDLLLNYLETELLQS | |
| hIL-10R | ESKYGPPCPPCPAPEAAGGPSVFLFPPKPKDTLMISRTPEVTCVVVDVSQED | 462 |
| BP-11 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMLSVMVSSSLVLIV | |
| | FFLGASEEAKPATTTIKNTKPQCRPEDYATRLQDLRVTFHRVKPTLQREDDY | |
| | SVWLDGTVVKGCWGCSVMDWLLRRYLEIVEPAGDHVYPGLKTELHSMRSTLE | |
| | SIYKDMRQCPLLGCGDKSVISRLSQEAERKSDNGTRKGLSELDTLFSRLEEY | |
| | LHSRK | |
| hIL-10R | ESKYGPPCPPCPAPEAAGGPSVFLFPPKPKDTLMISRTPEVTCVVVDVSQED | 463 |
| BP-12 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGGGGGSMALAHQLPVWIFSI | |
| | WILYFTLPLSEERVLPLRGNCKLLLQDTVIPNLLYSMRSIFQDIKPYFQGKD | |
| | SLNNLLLSGQLLEDLQSPIGCDALSEMIQFYLEEVMPQAEIHHPKHKNSVMQ | |
| | LGETLHTLISQLQECTALFPCKHKSLGAQKIKEEVSKLGQYGIIKAVAEFDI | |
| | FINYMESYFGVK | |
| hIL-10R | ESKYGPPCPPCPAPEAAGGPSVFLFPPKPKDTLMISRTPEVTCVVVDVSQED | 464 |
| BP-13 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMRRRRRSEGIIVAG | |
| | AIGTLLMMAVVVLSAHDHEHKEVPPACDPVHGNLAGIFKELRATYASIREGL | |
| | QKKDTVYYTSLENDRVLHEMLSPMGCRVTNELMEHYLDGVLPRASHLDYDNS | |
| | TLNGLHVFASSMQALYQHMLKCPALACTGKTPAWMYFLEVEHKLNPWRGTAK | |
| | AAAEADLLLNYLETFLLQF | |
| hIL-10R | ESKYGPPCPPCPAPEAAGGPSVFLFPPKPKDTLMISRTPEVTCVVVDVSQED | 465 |
| BP-14 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSMRRRRSFGIVVAGA | |
| | IGTLLMMAVVVESAHEHKEVPPACDPVHGNLAGIFKELRATYASIREGLQKK | |
| | DTVYYTSLFNDRVLQEMLSPMGCRVTNELMEHYLDGVLPRALHLDYDNSTLN | |
| | GLHAFASSMQALYQHMLKCPALACTGKTPAWMYFLEVEHKLNPWRGTAKAAA | |
| | EADLLLNYLETFLLQF | |
| hIL-10R | MYRMQLLSCIALSLALVTNSAESKYGPPCPPCPAPEAAGGPSVFLFPPKPKD | 506 |
| BP-1 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVESCSVMHEALHNHYTQKSLSLSLGGGGGGGGSGGG | |
| | GSGGGGSSPGQGTQSENSCTHFPGNLPNMLRDLRDAFSRVKTFFQMKDQLDN | |
| | LLLKESLLEDFKGYLGCQALSEMIQFYLEEVMPQAENQDPDIKAHVNSLGEN | |
| | LKTLRLRLRRCHRFLPCENKSKAVEQVKNAFNKLQEKGIYKAMSEFDIFINY | |
| | IEAYMTMKIRN | |
| m2a-hIL- | MYRMQLLSCIALSLALVINSEPRGPTIKPCPPCKCPAPNAAGGPSVFIFPPK | 507 |
| 10R BP-1 | IKDVLMISLSPIVTCVVVDVSEDDPDVQISWFVNNVEVHTAQTQTHREDYNS | |
| | TLRVVSALPIQHQDWMSGKEFKCKVNNKDLGAPIERTISKPKGSVRAPQVYV | |
| | LPPPEEEMTKKQVTLTCMVTDEMPEDIYVEWTNNGKTELNYKNTEPVLDSDG | |
| | SYFMYSKLRVEKKNWVERNSYSCSVVHEGLHNHHTTKSFSRTPGKGGGGGGG | |
| | SGGGGSGGGGSSPGQGTQSENSCTHEPGNLPNMLRDLRDAFSRVKTFFQMKD | |
| | QLDNLLLKESLLEDFKGYLGCQALSEMIQFYLEEVMPQAENQDPDIKAHVNS | |
| | LGENLKTLRLRLRRCHRFLPCENKSKAVEQVKNAFNKLQEKGIYKAMSEFDI | |
| | FINYIEAYMTMKIRN | |
| m1-hIL-10R | MYRMQLLSCIALSLALVTNSVPRDCGCKPCICTVPEVSSVFIFPPKPKDVLT | 508 |
| BP-1 | ITLTPKVTCVVVAISKDDPEVQFSWFVDDVEVHTAQTQPREEQENSTERSVS | |
| | ELPIMHQDWLNGKEFKCRVNSAAFPAPIEKTISKTKGRPKAPQVYTIPPPKE | |
| | QMAKDKVSLTCMITDFFPEDITVEWQWNGQPAENYKNTQPIMDTDGSYFVYS | |
| | KLNVQKSNWEAGNTFTCSVLHEGLHNHHTEKSLSHSPGGGGGGGSGGGGSGG | |
| | GGSSPGQGTQSENSCTHFPGNLPNMLRDLRDAFSRVKTFFQMKDQLDNLLLK | |
| | ESLLEDFKGYLGCQALSEMIQFYLEEVMPQAENQDPDIKAHVNSLGENLKTL | |
| | RLRLRRCHRFLPCENKSKAVEQVKNAFNKLQEKGIYKAMSEFDIFINYIEAY | |
| | MTMKIRN | |
| hIL-10R | MYRMQLLSCIALSLALVTNSAESKYGPPCPPCPAPEAAGGPSVFLFPPKPKD | 509 |
| BP-2 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSSPGQGTQSENSCTHFPGYLPNMLRDLRDAFSRVKTFFQMKDQLDN | |
| | LLLKESLLEDFKGYLGCQALSEMIQFYLEEVMPQAENQDPDIKAHVQSLGEN | |
| | LKDLRLWLRRCHRFLPCENKSKAVEQVKNAFNKLQEKGIYKAMSEFDIFINY | |
| | IEAYMTMKIRN | |
| m2a-hIL- | MYRMQLLSCIALSLALVTNSEPRGPTIKPCPPCKCPAPNAAGGPSVFIFPPK | 510 |
| 10R BP-2 | IKDVLMISLSPIVTCVVVDVSEDDPDVQISWFVNNVEVHTAQTQTHREDYNS | |
| with hIL-2 | TLRVVSALPIQHQDWMSGKEFKCKVNNKDLGAPIERTISKPKGSVRAPQVYV | |
| signal | LPPPEEEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGGGGGGG | |
| | SGGGGSGGGGSSPGQGTQSENSCTHEPGYLPNMLRDLRDAFSRVKTFFQMKD | |
| | QLDNLLLKESLLEDFKGYLGCQALSEMIQFYLEEVMPQAENQDPDIKAHVQS | |
| | LGENLKDLRLWLRRCHRFLPCENKSKAVEQVKNAFNKLQEKGIYKAMSEFDI | |
| | FINYIEAYMTMKIRN | |
| m1-hIL-10R | MYRMQLLSCIALSLALVTNSVPRDCGCKPCICTVPEVSSVFIFPPKPKDVLT | 511 |
| BP-2 | ITLTPKVTCVVVAISKDDPEVQFSWFVDDVEVHTAQTQPREEQENSTERSVS | |
| with hIL-2 | ELPIMHQDWLNGKEFKCRVNSAAFPAPIEKTISKTKGRPKAPQVYTIPPPKE | |
| signal | QMAKDKVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGGGGSGG | |
| | GGSSPGQGTQSENSCTHFPGYLPNMLRDLRDAFSRVKTFFQMKDQLDNLLLK | |
| | ESLLEDFKGYLGCQALSEMIQFYLEEVMPQAENQDPDIKAHVQSLGENLKDL | |
| | RLWLRRCHRFLPCENKSKAVEQVKNAFNKLQEKGIYKAMSEFDIFINYIEAY | |
| | MTMKIRN | |
| hIL-10R | MYRMQLLSCIALSLALVTNSAESKYGPPCPPCPAPEAAGGPSVFLFPPKPKD | 512 |
| BP-4 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSRHCMFGDSLRNSPDMKNMLQDLRGGYSGSGIKRTFQGKDTLDSML | |
| | LTQSLLDDFKGYLGCQALSEMIQFYLEEVMPQAENHGPTDSVKQLGEKLHTL | |
| | NQKFGECPRWFPCYYNTTPAVENVKSVESKLQERGVYKAMSEFDIFINYIET | |
| | YTTMK | |
| hIL-10R | MYRMQLLSCIALSLALVTNSAESKYGPPCPPCPAPEAAGGPSVFLFPPKPKD | 513 |
| BP-5 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSCQLESGEALPLGSRSADSRSVDGQRVPAPQNNYPGLLRDLRLGYE | |
| | GFKQKVTDSHPDETLLGSSRLAGDLKGPLRCQALSEMIQFLLQVVLPDAENS | |
| | RQDLRSQFSTLGDRITGLRQQLRRDPTVFPCESRSDGVSDLRSAYTRLGSTG | |
| | AEKVLSEFDIFINYIEAYVTSV | |
| hIL-10R | MYRMQLLSCIALSLALVTNSAESKYGPPCPPCPAPEAAGGPSVFLFPPKPKD | 514 |
| BP-6 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSYCVEYAESDEDRQQCSSSSNFPASLPHMLRELRAAFGKVKTFFQM | |
| | KDQLNSMLLTQSLLDDFKGYLGCQALSEMIQFYLEEVMPQAENHGPDIKEHV | |
| | NSLGEKLKTLRLRLRRCHRFLPCENKSKAVEQVKRVENMLQERGVYKAMSEF | |
| | DIFINYIESYMTTKM | |
| hIL-10R | MYRMQLLSCIALSLALVTNSAESKYGPPCPPCPAPEAAGGPSVFLFPPKPKD | 515 |
| BP-7 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSYCTSCSHHQCTEDENQKQDCEDANHSLPHMLRELRAAFGKVKTFF | |
| | QMKDQLHSLLLTQSLLDDFKGYLGCQALSEMIQFYLEEVMPQAENHGPEEHD | |
| | NSLSEHGPDVKEHVNSLGEKLKTLRLRLRRCHRFLPCENKSKAVEKVKRVES | |
| | ELQERGVYKAMSEFDIFINYIETYMTT | |
| hIL-10R | MYRMQLLSCIALSLALVINSAESKYGPPCPPCPAPEAAGGPSVFLFPPKPKD | 516 |
| BP-8 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSRSPKNKPSIDCNPQTGDFVNMLKSMRQDYSRIRDTLHDRDKLHSS | |
| | LLTGALLDEMMGYSGCRTTLLLMEHYLDTWYPAAYRHHLYDNQTLVVVDRMG | |
| | STLVALLKAMVQCPMLACGAPSPAMDKMLQQEAKMKKYTGVYKGISETDLLL | |
| | GYLELYMMKEKR | |
| hIL-10R | MYRMQLLSCIALSLALVTNSAESKYGPPCPPCPAPEAAGGPSVFLFPPKPKD | 517 |
| BP-9 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSHEHKVPPACDPVHGNLAGIFKELRAIYASIREALQKKDTVYYTSL | |
| | FNDRVLQEMLSPMGCRVTNELMEHYLDGVLPRAAHFDYDNSTLNGLHAFTSS | |
| | MQALYQHMLKCPALACTGKTPAWMYFLEVEHKLNPWRGTAKAAAEADLLLNY | |
| | LETFLLQF | |
| hIL-10R | MYRMQLLSCIALSLALVTNSAESKYGPPCPPCPAPEAAGGPSVFLFPPKPKD | 518 |
| BP-10 | TLMISRTPEVTCVVVDVSQEDPEVQFNWYVDGVEVHNAKTKPREEQENSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSKGRDSKPSPACDPMHGALAGIFKELRTTYRSVREALQTKDTVYYV | |
| | SLFHEQLLQEMLSPVGCRVTNELMQHYLDGVLPRAFHCGYDNATLNALHALS | |
| | SSLSTLYQHMLKCPALACTGQTPAWTQFLDTEHKLDPWKGTVKATAEMDLLL | |
| | NYLETELLQS | |
| m2a-hIL- | MYRMQLLSCIALSLALVTNSEPRGPTIKPCPPCKCPAPNAAGGPSVFIFPPK | 519 |
| 10R BP-10 | IKDVLMISLSPIVTCVVVDVSEDDPDVQISWFVNNVEVHTAQTQTHREDYNS | |
| with hIL-2 | TLRVVSALPIQHQDWMSGKEFKCKVNNKDLGAPIERTISKPKGSVRAPQVYV | |
| signal | LPPPEEEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGGGGGGG | |
| | GSGGGGSGGGGSKGRDSKPSPACDPMHGALAGIFKELRTTYRSVREALQTKD | |
| | TVYYVSLFHEQLLQEMLSPVGCRVTNELMQHYLDGVLPRAFHCGYDNATLNA | |
| | LHALSSSLSTLYQHMLKCPALACTGQTPAWTQFLDTEHKLDPWKGTVKATAE | |
| | MDLLLNYLETELLQS | |
| m1-hIL-10R | MYRMQLLSCIALSLALVTNSVPRDCGCKPCICTVPEVSSVFIFPPKPKDVLT | 520 |
| BP-10 | ITLTPKVTCVVVAISKDDPEVQFSWFVDDVEVHTAQTQPREEQENSTERSVS | |
| with hIL-2 | ELPIMHQDWLNGKEFKCRVNSAAFPAPIEKTISKTKGRPKAPQVYTIPPPKE | |
| signal | QMAKDKVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGGGGSGG | |
| | GGSKGRDSKPSPACDPMHGALAGIFKELRTTYRSVREALQTKDTVYYVSLFH | |
| | EQLLQEMLSPVGCRVTNELMQHYLDGVLPRAFHCGYDNATLNALHALSSSLS | |
| | TLYQHMLKCPALACTGQTPAWTQFLDTEHKLDPWKGTVKATAEMDLLLNYLE | |
| | TELLQS | |
| hIL-10R | MYRMQLLSCIALSLALVTNSAESKYGPPCPPCPAPEAAGGPSVFLFPPKPKD | 521 |
| BP-11 | TLMISRTPEVTCVVVDVSQEDPEVQFNWYVDGVEVHNAKTKPREEQFNSTYR | |
| with hIL-2 | VVSVLTVLHQDWLNGKEYKCKVSNKGLPSSIEKTISKAKGQPREPQVYTLPP | |
| signal | SQEEMTKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSLGGGGGGGGSGGG | |
| | GSGGGGSATTTIKNTKPQCRPEDYATRLQDLRVTFHRVKPTLQREDDYSVWL | |
| | DGTVVKGCWGCSVMDWLLRRYLEIVFPAGDHVYPGLKTELHSMRSTLESIYK | |
| | DMRQCPLLGCGDKSVISRLSQEAERKSDNGTRKGLSELDTLFSRLEEYLHSR | |
| | K | |
| hIL-10R | AESKYGPPCPPCPAPEAAGGPSVFLFPPKPKDTLMISRTPEVTCVVVDVSQE | 522 |
| BP-1 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSSPGQGTQSENSCT | |
| | HFPGNLPNMLRDLRDAFSRVKTFFQMKDQLDNLLLKESLLEDFKGYLGCQAL | |
| | SEMIQFYLEEVMPQAENQDPDIKAHVNSLGENLKTLRLRLRRCHRFLPCENK | |
| | SKAVEQVKNAFNKLQEKGIYKAMSEFDIFINYIEAYMTMKIRN | |
| m2a-hIL- | EPRGPTIKPCPPCKCPAPNAAGGPSVFIFPPKIKDVLMISLSPIVTCVVVDV | 523 |
| 10R BP-1 | SEDDPDVQISWFVNNVEVHTAQTQTHREDYNSTLRVVSALPIQHQDWMSGKE | |
| without hIL- | FKCKVNNKDLGAPIERTISKPKGSVRAPQVYVLPPPEEEMTKKQVTLTCMVT | |
| 2 signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGSGGGGSGGGGSSPGQGTQSE | |
| | NSCTHEPGNLPNMLRDLRDAFSRVKTFFQMKDQLDNLLLKESLLEDEKGYLG | |
| | CQALSEMIQFYLEEVMPQAENQDPDIKAHVNSLGENLKTLRLRLRRCHRELP | |
| | CENKSKAVEQVKNAFNKLQEKGIYKAMSEFDIFINYIEAYMTMKIRN | |
| m1-hIL-10R | VPRDCGCKPCICTVPEVSSVFIFPPKPKDVLTITLTPKVTCVVVAISKDDPE | 524 |
| BP-1 | VQFSWFVDDVEVHTAQTQPREEQFENSTERSVSELPIMHQDWLNGKEFKCRVN | |
| without hIL- | SAAFPAPIEKTISKTKGRPKAPQVYTIPPPKEQMAKDKVSLTCMITDFFPED | |
| 2 signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGGGGGSSPGQGTQSENSCTHFPG | |
| | NLPNMLRDLRDAFSRVKTFFQMKDQLDNLLLKESLLEDFKGYLGCQALSEMI | |
| | QFYLEEVMPQAENQDPDIKAHVNSLGENLKTLRLRLRRCHRFLPCENKSKAV | |
| | EQVKNAFNKLQEKGIYKAMSEFDIFINYIEAYMTMKIRN | |
| hIL-10R | AESKYGPPCPPCPAPEAAGGPSVFLFPPKPKDTLMISRTPEVTCVVVDVSQE | 525 |
| BP-2 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSSPGQGTQSENSCT | |
| | HFPGYLPNMLRDLRDAFSRVKTFFQMKDQLDNLLLKESLLEDFKGYLGCQAL | |
| | SEMIQFYLEEVMPQAENQDPDIKAHVQSLGENLKDLRLWLRRCHRELPCENK | |
| | SKAVEQVKNAFNKLQEKGIYKAMSEFDIFINYIEAYMTMKIRN | |
| m2a-hIL- | EPRGPTIKPCPPCKCPAPNAAGGPSVFIFPPKIKDVLMISLSPIVTCVVVDV | 526 |
| 10R BP-2 | SEDDPDVQISWFVNNVEVHTAQTQTHREDYNSTLRVVSALPIQHQDWMSGKE | |
| without hIL- | FKCKVNNKDLGAPIERTISKPKGSVRAPQVYVLPPPEEEMTKKQVTLTCMVT | |
| 2 signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGSGGGGSGGGGSSPGQGTQSE | |
| | NSCTHEPGYLPNMLRDLRDAFSRVKTFFQMKDQLDNLLLKESLLEDEKGYLG | |
| | CQALSEMIQFYLEEVMPQAENQDPDIKAHVQSLGENLKDLRLWLRRCHRELP | |
| | CENKSKAVEQVKNAFNKLQEKGIYKAMSEFDIFINYIEAYMTMKIRN | |
| m1-hIL-10R | VPRDCGCKPCICTVPEVSSVFIFPPKPKDVLTITLTPKVTCVVVAISKDDPE | 527 |
| BP-2 | VQFSWFVDDVEVHTAQTQPREEQFNSTERSVSELPIMHQDWLNGKEFKCRVN | |
| without hIL- | SAAFPAPIEKTISKTKGRPKAPQVYTIPPPKEQMAKDKVSLTCMITDFFPED | |
| 2 signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGSGGGGSSPGQGTQSENSCTHEPG | |
| | YLPNMLRDLRDAFSRVKTFFQMKDQLDNLLLKESLLEDFKGYLGCQALSEMI | |
| | QFYLEEVMPQAENQDPDIKAHVQSLGENLKDLRLWLRRCHRFLPCENKSKAV | |
| | EQVKNAFNKLQEKGIYKAMSEFDIFINYIEAYMTMKIRN | |
| hIL-10R | AESKYGPPCPPCPAPEAAGGPSVFLFPPKPKDTLMISRTPEVTCVVVDVSQE | 528 |
| BP-4 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSRHCMFGDSLRNSP | |
| | DMKNMLQDLRGGYSGSGIKRTFQGKDTLDSMLLTQSLLDDEKGYLGCQALSE | |
| | MIQFYLEEVMPQAENHGPTDSVKQLGEKLHTLNQKFGECPRWFPCYYNTTPA | |
| | VENVKSVFSKLQERGVYKAMSEFDIFINYIETYTTMK | |
| hIL-10R | AESKYGPPCPPCPAPEAAGGPSVFLFPPKPKDTLMISRTPEVTCVVVDVSQE | 529 |
| BP-5 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSCQLESGEALPLGS | |
| | RSADSRSVDGQRVPAPQNNYPGLLRDLRLGYEGFKQKVTDSHPDETLLGSSR | |
| | LAGDLKGPLRCQALSEMIQFLLQVVLPDAENSRQDLRSQFSTLGDRITGLRQ | |
| | QLRRDPTVFPCESRSDGVSDLRSAYTRLGSTGAEKVLSEFDIFINYIEAYVT | |
| | SV | |
| hIL-10R | AESKYGPPCPPCPAPEAAGGPSVFLFPPKPKDTLMISRTPEVTCVVVDVSQE | 530 |
| BP-6 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSYCVEYAESDEDRQ | |
| | QCSSSSNFPASLPHMLRELRAAFGKVKTFFQMKDQLNSMLLTQSLLDDEKGY | |
| | LGCQALSEMIQFYLEEVMPQAENHGPDIKEHVNSLGEKLKTLRLRLRRCHRE | |
| | LPCENKSKAVEQVKRVENMLQERGVYKAMSEFDIFINYIESYMTTKM | |
| hIL-10R | AESKYGPPCPPCPAPEAAGGPSVFLFPPKPKDTLMISRTPEVTCVVVDVSQE | 531 |
| BP-7 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVFSC | |
| peptide | SVMHEALHNHYTQKSLSLSLGGGGGGGGSGGGGSGGGGSYCTSCSHHQCTED | |
| | ENQKQDCEDANHSLPHMLRELRAAFGKVKTFFQMKDQLHSLLLTQSLLDDEK | |
| | GYLGCQALSEMIQFYLEEVMPQAENHGPEEHDNSLSEHGPDVKEHVNSLGEK | |
| | LKTLRLRLRRCHRFLPCENKSKAVEKVKRVESELQERGVYKAMSEFDIFINY | |
| | IETYMTT | |
| hIL-10R | AESKYGPPCPPCPAPEAAGGPSVFLFPPKPKDTLMISRTPEVTCVVVDVSQE | 532 |
| BP-8 | DPEVQFNWYVDGVEVHNAKTKPREEQFNSTYRVVSVLTVLHQDWLNGKEYKC | |
| with hIL-2 | KVSNKGLPSSIEKTISKAKGQPREPQVYTLPPSQEEMTKNQVSLTCLVKGFY | |
| signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSRSPKNKPSIDCNP | |
| | QTGDFVNMLKSMRQDYSRIRDTLHDRDKLHSSLLTGALLDEMMGYSGCRTTL | |
| | LLMEHYLDTWYPAAYRHHLYDNQTLVVVDRMGSTLVALLKAMVQCPMLACGA | |
| | PSPAMDKMLQQEAKMKKYTGVYKGISETDLLLGYLELYMMKEKR | |
| hIL-10R | AESKYGPPCPPCPAPEAAGGPSVFLFPPKPKDTLMISRTPEVTCVVVDVSQE | 533 |
| BP-9 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSHEHKVPPACDPVH | |
| | GNLAGIFKELRAIYASIREALQKKDTVYYTSLENDRVLQEMLSPMGCRVTNE | |
| | LMEHYLDGVLPRAAHFDYDNSTLNGLHAFTSSMQALYQHMLKCPALACTGKT | |
| | PAWMYFLEVEHKLNPWRGTAKAAAEADLLLNYLETFLLQF | |
| hIL-10R | AESKYGPPCPPCPAPEAAGGPSVFLFPPKPKDTLMISRTPEVTCVVVDVSQE | 534 |
| BP-10 | DPEVQFNWYVDGVEVHNAKTKPREEQFNSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSKGRDSKPSPACDP | |
| | MHGALAGIFKELRTTYRSVREALQTKDTVYYVSLFHEQLLQEMLSPVGCRVT | |
| | NELMQHYLDGVLPRAFHCGYDNATLNALHALSSSLSTLYQHMLKCPALACTG | |
| | QTPAWTQFLDTEHKLDPWKGTVKATAEMDLLLNYLETFLLQS | |
| m2a-hIL- | EPRGPTIKPCPPCKCPAPNAAGGPSVFIFPPKIKDVLMISLSPIVTCVVVDV | 535 |
| 10R BP-10 | SEDDPDVQISWFVNNVEVHTAQTQTHREDYNSTLRVVSALPIQHQDWMSGKE | |
| with hIL-2 | FKCKVNNKDLGAPIERTISKPKGSVRAPQVYVLPPPEEEMTKKQVTLTCMVT | |
| signal | DEMPEDIYVEWTNNGKTELNYKNTEPVLDSDGSYFMYSKLRVEKKNWVERNS | |
| peptide | YSCSVVHEGLHNHHTTKSFSRTPGKGGGGGGGGSGGGGSGGGGSKGRDSKPS | |
| | PACDPMHGALAGIFKELRTTYRSVREALQTKDTVYYVSLFHEQLLQEMLSPV | |
| | GCRVTNELMQHYLDGVLPRAFHCGYDNATLNALHALSSSLSTLYQHMLKCPA | |
| | LACTGQTPAWTQFLDTEHKLDPWKGTVKATAEMDLLLNYLETELLQS | |
| m1-hIL-10R | VPRDCGCKPCICTVPEVSSVFIFPPKPKDVLTITLTPKVTCVVVAISKDDPE | 536 |
| BP-10 | VQFSWFVDDVEVHTAQTQPREEQFNSTERSVSELPIMHQDWLNGKEFKCRVN | |
| with hIL-2 | SAAFPAPIEKTISKTKGRPKAPQVYTIPPPKEQMAKDKVSLTCMITDFFPED | |
| signal | ITVEWQWNGQPAENYKNTQPIMDTDGSYFVYSKLNVQKSNWEAGNTFTCSVL | |
| peptide | HEGLHNHHTEKSLSHSPGGGGGGGSGGGGSGGGGSKGRDSKPSPACDPMHGA | |
| | LAGIFKELRTTYRSVREALQTKDTVYYVSLFHEQLLQEMLSPVGCRVTNELM | |
| | QHYLDGVLPRAFHCGYDNATLNALHALSSSLSTLYQHMLKCPALACTGQTPA | |
| | WTQFLDTEHKLDPWKGTVKATAEMDLLLNYLETELLQS | |
| hIL-10R | AESKYGPPCPPCPAPEAAGGPSVFLFPPKPKDTLMISRTPEVTCVVVDVSQE | 537 |
| BP-11 | DPEVQFNWYVDGVEVHNAKTKPREEQENSTYRVVSVLTVLHQDWLNGKEYKC | |
| without hIL- | KVSNKGLPSSIEKTISKAKGQPREPQVYTLPPSQEEMTKNQVSLTCLVKGFY | |
| 2 signal | PSDIAVEWESNGQPENNYKTTPPVLDSDGSFFLYSRLTVDKSRWQEGNVESC | |
| peptide | SVMHEALHNHYTQKSLSLSLGGGGGGGGSGGGGSGGGGSATTTIKNTKPQCR | |
| | PEDYATRLQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDKSVISRLS | |
| | QEAERKSDNGTRKGLSELDTLFSRLEEYLHSRK | |
| hIL-10R | MYRMQLLSCIALSLALVTNSESKYGPPCPPCPAPEAAGGPSVFLFPPKPKDT | 538 |
| BP-1 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSSPGQGTQSENSCTHEPGNLPNMLRDLRDAFSRVKTFFQMKDQLDNL | |
| | LLKESLLEDFKGYLGCQALSEMIQFYLEEVMPQAENQDPDIKAHVNSLGENL | |
| | KTLRLRLRRCHRFLPCENKSKAVEQVKNAFNKLQEKGIYKAMSEFDIFINYI | |
| | EAYMTMKIRN | |
| hIL-10R | MYRMQLLSCIALSLALVTNSESKYGPPCPPCPAPEAAGGPSVFLFPPKPKDT | 539 |
| BP-2 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSSPGQGTQSENSCTHFPGYLPNMLRDLRDAFSRVKTFFQMKDQLDNL | |
| | LLKESLLEDFKGYLGCQALSEMIQFYLEEVMPQAENQDPDIKAHVQSLGENL | |
| | KDLRLWLRRCHRFLPCENKSKAVEQVKNAFNKLQEKGIYKAMSEFDIFINYI | |
| | EAYMTMKIRN | |
| hIL-10R | MYRMQLLSCIALSLALVTNSESKYGPPCPPCPAPEAAGGPSVFLFPPKPKDT | 540 |
| BP-4 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSRHCMFGDSLRNSPDMKNMLQDLRGGYSGSGIKRTFQGKDTLDSMLL | |
| | TQSLLDDFKGYLGCQALSEMIQFYLEEVMPQAENHGPTDSVKQLGEKLHTLN | |
| | QKFGECPRWFPCYYNTTPAVENVKSVESKLQERGVYKAMSEFDIFINYIETY | |
| | TTMK | |
| hIL-10R | MYRMQLLSCIALSLALVTNSESKYGPPCPPCPAPEAAGGPSVFLFPPKPKDT | 541 |
| BP-5 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFFL | |
| peptide | YSRLTVDKSRWQEGNVESCSVMHEALHNHYTQKSLSLSLGGGGGGGGSGGGG | |
| | SGGGGSCQLESGEALPLGSRSADSRSVDGQRVPAPQNNYPGLLRDLRLGYEG | |
| | FKQKVTDSHPDETLLGSSRLAGDLKGPLRCQALSEMIQFLLQVVLPDAENSR | |
| | QDLRSQFSTLGDRITGLRQQLRRDPTVFPCESRSDGVSDLRSAYTRLGSTGA | |
| | EKVLSEFDIFINYIEAYVTSV | |
| hIL-10R | MYRMQLLSCIALSLALVTNSESKYGPPCPPCPAPEAAGGPSVFLFPPKPKDT | 542 |
| BP-6 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSYCVEYAESDEDRQQCSSSSNEPASLPHMLRELRAAFGKVKTFFQMK | |
| | DQLNSMLLTQSLLDDFKGYLGCQALSEMIQFYLEEVMPQAENHGPDIKEHVN | |
| | SLGEKLKTLRLRLRRCHRFLPCENKSKAVEQVKRVENMLQERGVYKAMSEED | |
| | IFINYIESYMTTKM | |
| hIL-10R | MYRMQLLSCIALSLALVTNSESKYGPPCPPCPAPEAAGGPSVFLFPPKPKDT | 543 |
| BP-7 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFFL | |
| peptide | YSRLTVDKSRWQEGNVFSCSVMHEALHNHYTQKSLSLSLGGGGGGGGSGGGG | |
| | SGGGGSYCTSCSHHQCTEDENQKQDCEDANHSLPHMLRELRAAFGKVKTFFQ | |
| | MKDQLHSLLLTQSLLDDFKGYLGCQALSEMIQFYLEEVMPQAENHGPEEHDN | |
| | SLSEHGPDVKEHVNSLGEKLKTLRLRLRRCHRFLPCENKSKAVEKVKRVESE | |
| | LQERGVYKAMSEFDIFINYIETYMTT | |
| hIL-10R | MYRMQLLSCIALSLALVINSESKYGPPCPPCPAPEAAGGPSVFLFPPKPKDT | 544 |
| BP-8 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSRSPKNKPSIDCNPQTGDFVNMLKSMRQDYSRIRDTLHDRDKLHSSL | |
| | LTGALLDEMMGYSGCRTTLLLMEHYLDTWYPAAYRHHLYDNQTLVVVDRMGS | |
| | TLVALLKAMVQCPMLACGAPSPAMDKMLQQEAKMKKYTGVYKGISETDLLLG | |
| | YLELYMMKEKR | |
| hIL-10R | MYRMQLLSCIALSLALVTNSESKYGPPCPPCPAPEAAGGPSVFLFPPKPKDT | 545 |
| BP-9 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSHEHKVPPACDPVHGNLAGIFKELRAIYASIREALQKKDTVYYTSLE | |
| | NDRVLQEMLSPMGCRVTNELMEHYLDGVLPRAAHFDYDNSTLNGLHAFTSSM | |
| | QALYQHMLKCPALACTGKTPAWMYFLEVEHKLNPWRGTAKAAAEADLLLNYL | |
| | ETFLLQF | |
| hIL-10R | MYRMQLLSCIALSLALVTNSESKYGPPCPPCPAPEAAGGPSVFLFPPKPKDT | 546 |
| BP-10 | LMISRTPEVTCVVVDVSQEDPEVQFNWYVDGVEVHNAKTKPREEQENSTYRV | |
| with hIL-2 | VSVLTVLHQDWLNGKEYKCKVSNKGLPSSIEKTISKAKGQPREPQVYTLPPS | |
| signal | QEEMTKNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGGSGGGG | |
| | SGGGGSKGRDSKPSPACDPMHGALAGIFKELRTTYRSVREALQTKDTVYYVS | |
| | LFHEQLLQEMLSPVGCRVTNELMQHYLDGVLPRAFHCGYDNATLNALHALSS | |
| | SLSTLYQHMLKCPALACTGQTPAWTQFLDTEHKLDPWKGTVKATAEMDLLLN | |
| | YLETELLQS | |
| hIL-10R | MYRMQLLSCIALSLALVINSESKYGPPCPPCPAPEAAGGPSVFLFPPKPKDT | 547 |
| BP-11 | LMISRTPEVTCVVVDVSQEDPEVQFNWYVDGVEVHNAKTKPREEQENSTYRV | |
| without hIL- | VSVLTVLHQDWLNGKEYKCKVSNKGLPSSIEKTISKAKGQPREPQVYTLPPS | |
| 2 signal | QEEMTKNQVSLTCLVKGFYPSDIAVEWESNGQPENNYKTTPPVLDSDGSFFL | |
| peptide | YSRLTVDKSRWQEGNVFSCSVMHEALHNHYTQKSLSLSLGGGGGGGGSGGGG | |
| | SGGGGSATTTIKNTKPQCRPEDYATRLQDLRVTFHRVKPTLQREDDYSVWLD | |
| | GTVVKGCWGCSVMDWLLRRYLEIVFPAGDHVYPGLKTELHSMRSTLESIYKD | |
| | MRQCPLLGCGDKSVISRLSQEAERKSDNGTRKGLSELDTLFSRLEEYLHSRK | |
| hIL-10R | ESKYGPPCPPCPAPEAAGGPSVFLFPPKPKDTLMISRTPEVTCVVVDVSQED | 548 |
| BP-1 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSSPGQGTQSENSCTH | |
| | FPGNLPNMLRDLRDAFSRVKTFFQMKDQLDNLLLKESLLEDEKGYLGCQALS | |
| | EMIQFYLEEVMPQAENQDPDIKAHVNSLGENLKTLRLRLRRCHRFLPCENKS | |
| | KAVEQVKNAFNKLQEKGIYKAMSEFDIFINYIEAYMTMKIRN | |
| hIL-10R | ESKYGPPCPPCPAPEAAGGPSVFLFPPKPKDTLMISRTPEVTCVVVDVSQED | 549 |
| BP-2 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSSPGQGTQSENSCTH | |
| | FPGYLPNMLRDLRDAFSRVKTFFQMKDQLDNLLLKESLLEDFKGYLGCQALS | |
| | EMIQFYLEEVMPQAENQDPDIKAHVQSLGENLKDLRLWLRRCHRFLPCENKS | |
| | KAVEQVKNAFNKLQEKGIYKAMSEFDIFINYIEAYMTMKIRN | |
| hIL-10R | ESKYGPPCPPCPAPEAAGGPSVFLFPPKPKDTLMISRTPEVTCVVVDVSQED | 550 |
| BP-4 | PEVQFNWYVDGVEVHNAKTKPREEQENSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSRHCMFGDSLRNSPD | |
| | MKNMLQDLRGGYSGSGIKRTFQGKDTLDSMLLTQSLLDDEKGYLGCQALSEM | |
| | IQFYLEEVMPQAENHGPTDSVKQLGEKLHTLNQKFGECPRWFPCYYNTTPAV | |
| | ENVKSVFSKLQERGVYKAMSEFDIFINYIETYTTMK | |
| hIL-10R | ESKYGPPCPPCPAPEAAGGPSVFLFPPKPKDTLMISRTPEVTCVVVDVSQED | 551 |
| BP-5 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSCQLESGEALPLGSR | |
| | SADSRSVDGQRVPAPQNNYPGLLRDLRLGYEGFKQKVTDSHPDETLLGSSRL | |
| | AGDLKGPLRCQALSEMIQFLLQVVLPDAENSRQDLRSQFSTLGDRITGLRQQ | |
| | LRRDPTVFPCESRSDGVSDLRSAYTRLGSTGAEKVLSEFDIFINYIEAYVTS | |
| | V | |
| hIL-10R | ESKYGPPCPPCPAPEAAGGPSVFLFPPKPKDTLMISRTPEVTCVVVDVSQED | 552 |
| BP-6 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSYCVEYAESDEDRQQ | |
| | CSSSSNEPASLPHMLRELRAAFGKVKTFFQMKDQLNSMLLTQSLLDDEKGYL | |
| | GCQALSEMIQFYLEEVMPQAENHGPDIKEHVNSLGEKLKTLRLRLRRCHREL | |
| | PCENKSKAVEQVKRVENMLQERGVYKAMSEFDIFINYIESYMTTKM | |
| hIL-10R | ESKYGPPCPPCPAPEAAGGPSVFLFPPKPKDTLMISRTPEVTCVVVDVSQED | 553 |
| BP-7 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSYCTSCSHHQCTEDE | |
| | NQKQDCEDANHSLPHMLRELRAAFGKVKTFFQMKDQLHSLLLTQSLLDDEKG | |
| | YLGCQALSEMIQFYLEEVMPQAENHGPEEHDNSLSEHGPDVKEHVNSLGEKL | |
| | KTLRLRLRRCHRFLPCENKSKAVEKVKRVESELQERGVYKAMSEFDIFINYI | |
| | ETYMTT | |
| hIL-10R | ESKYGPPCPPCPAPEAAGGPSVFLFPPKPKDTLMISRTPEVTCVVVDVSQED | 554 |
| BP-8 | PEVQFNWYVDGVEVHNAKTKPREEQFNSTYRVVSVLTVLHQDWLNGKEYKCK | |
| with hIL-2 | VSNKGLPSSIEKTISKAKGQPREPQVYTLPPSQEEMTKNQVSLTCLVKGFYP | |
| signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSRSPKNKPSIDCNPQ | |
| | TGDFVNMLKSMRQDYSRIRDTLHDRDKLHSSLLTGALLDEMMGYSGCRTTLL | |
| | LMEHYLDTWYPAAYRHHLYDNQTLVVVDRMGSTLVALLKAMVQCPMLACGAP | |
| | SPAMDKMLQQEAKMKKYTGVYKGISETDLLLGYLELYMMKEKR | |
| hIL-10R | ESKYGPPCPPCPAPEAAGGPSVFLFPPKPKDTLMISRTPEVTCVVVDVSQED | 555 |
| BP-9 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSHEHKVPPACDPVHG | |
| | NLAGIFKELRAIYASIREALQKKDTVYYTSLENDRVLQEMLSPMGCRVTNEL | |
| | MEHYLDGVLPRAAHFDYDNSTLNGLHAFTSSMQALYQHMLKCPALACTGKTP | |
| | AWMYFLEVEHKLNPWRGTAKAAAEADLLLNYLETELLQF | |
| hIL-10R | ESKYGPPCPPCPAPEAAGGPSVFLFPPKPKDTLMISRTPEVTCVVVDVSQED | 556 |
| BP-10 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSKGRDSKPSPACDPM | |
| | HGALAGIFKELRTTYRSVREALQTKDTVYYVSLFHEQLLQEMLSPVGCRVTN | |
| | ELMQHYLDGVLPRAFHCGYDNATLNALHALSSSLSTLYQHMLKCPALACTGQ | |
| | TPAWTQFLDTEHKLDPWKGTVKATAEMDLLLNYLETELLQS | |
| hIL-10R | ESKYGPPCPPCPAPEAAGGPSVFLFPPKPKDTLMISRTPEVTCVVVDVSQED | 557 |
| BP-11 | PEVQFNWYVDGVEVHNAKTKPREEQFNSTYRVVSVLTVLHQDWLNGKEYKCK | |
| without hIL- | VSNKGLPSSIEKTISKAKGQPREPQVYTLPPSQEEMTKNQVSLTCLVKGFYP | |
| 2 signal | SDIAVEWESNGQPENNYKTTPPVLDSDGSFFLYSRLTVDKSRWQEGNVESCS | |
| peptide | VMHEALHNHYTQKSLSLSLGGGGGGGGSGGGGSGGGGSATTTIKNTKPQCRP | |
| | EDYATRLQDLRVTFHRVKPTLQREDDYSVWLDGTVVKGCWGCSVMDWLLRRY | |
| | LEIVFPAGDHVYPGLKTELHSMRSTLESIYKDMRQCPLLGCGDKSVISRLSQ | |
| | EAERKSDNGTRKGLSELDTLFSRLEEYLHSRK | |

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 7.

In some embodiments, the amino acid sequence of the hIL-10R binding fusion protein comprises the amino acid sequence of a polypeptide set forth in Table 7, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence of a polypeptide set forth in Table 7, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence of a polypeptide set forth in Table 7, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence of a polypeptide set forth in Table 7, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence of a polypeptide set forth in Table 7, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 7.

In some embodiments, the amino acid sequence of the hIL-10R binding fusion protein consists of the amino acid sequence of a polypeptide set forth in Table 7, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence of a polypeptide set forth in Table 7, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence of a polypeptide set forth in Table 7, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence of a polypeptide set forth in Table 7, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence of a polypeptide set forth in Table 7, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412-417 or 420-465 or 494-557.

In some embodiments, the amino acid sequence of the hIL-10R binding fusion protein comprises the amino acid sequence set forth in any one of SEQ ID NOS: 412-417 or 420-465 or 494-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-417 or 420-465 or 494-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-417 or 420-465 or 494-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-417 or 420-465 or 494-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-417 or 420-465 or 494-557, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412-417 or 420-465 or 494-557.

In some embodiments, the amino acid sequence of the hIL-10R binding fusion protein consists of the amino acid sequence set forth in any one of SEQ ID NOS: 412-417 or 420-465 or 494-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-417 or 420-465 or 494-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-417 or 420-465 or 494-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-417 or 420-465 or 494-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-417 or 420-465 or 494-557, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412-415 or 494-497 or 500-503.

In some embodiments, the amino acid sequence of the hIL-10R binding fusion protein comprises the amino acid sequence set forth in any one of SEQ ID NOS: 412-415 or 494-497 or 500-503, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-415 or 494-497 or 500-503, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-415 or 494-497 or 500-503, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-415 or 494-497 or 500-503, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412-415 or 494-497 or 500-503, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412-415 or 494-497 or 500-503.

In some embodiments, the amino acid sequence of the hIL-10R binding fusion protein consists of the amino acid sequence set forth in any one of SEQ ID NOS: 412-415 or 494-497 or 500-503, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-415 or 494-497 or 500-503, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-415 or 494-497 or 500-503, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-415 or 494-497 or 500-503, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412-415 or 494-497 or 500-503, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 416-417 or 420-465 or 498-499 or 504-505.

In some embodiments, the amino acid sequence of the hIL-10R binding fusion protein comprises the amino acid sequence set forth in any one of SEQ ID NOS: 416-417 or 420-465 or 498-499 or 504-505, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 416-417 or 420-465 or 498-499 or 504-505, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 416-417 or 420-465 or 498-499 or 504-505, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 416-417 or 420-465 or 498-499 or 504-505, and further consist of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 416-417 or 420-465 or 498-499 or 504-505, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 416-417 or 420-465 or 498-499 or 504-505.

In some embodiments, the amino acid sequence of the hIL-10R binding fusion protein consists of the amino acid sequence set forth in any one of SEQ ID NOS: 416-417 or 420-465 or 498-499 or 504-505, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 416-417 or 420-465 or 498-499 or 504-505, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 416-417 or 420-465 or 498-499 or 504-505, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 416-417 or 420-465 or 498-499 or 504-505, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 416-417 or 420-465 or 498-499 or 504-505, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412, 414, 416, 420-429 or 494-499.

In some embodiments, the amino acid sequence of the hIL-10R binding fusion protein comprises the amino acid sequence set forth in any one of SEQ ID NOS: 412, 414, 416, 420-429 or 494-499, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412, 414, 416, 420-429 or 494-499, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412, 414, 416, 420-429 or 494-499, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412, 414, 416, 420-429 or 494-499, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 412, 414, 416, 420-429 or 494-499, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 412, 414, 416, 420-429 or 494-499.

In some embodiments, the amino acid sequence of the hIL-10R binding fusion protein consists of the amino acid sequence set forth in any one of SEQ ID NOS: 412, 414, 416, 420-429 or 494-499, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412, 414, 416, 420-429 or 494-499, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412, 414, 416, 420-429 or 494-499, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412, 414, 416, 420-429 or 494-499, and further consists about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 412, 414, 416, 420-429 or 494-499, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 413, 415, 417, 430-465 or 500-439.

In some embodiments, the amino acid sequence of the hIL-10R binding fusion protein comprises the amino acid sequence set forth in any one of SEQ ID NOS: 430-465 or 500-439, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 430-465 or 500-439, and further comprises or consists of at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 430-465 or 500-439, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 430-465 or 500-439, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 430-465 or 500-439, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 413, 415, 417, 430-465 or 500-439.

In some embodiments, the amino acid sequence of the hIL-10R binding fusion protein consists of the amino acid sequence set forth in any one of SEQ ID NOS: 430-465 or 500-439, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 430-465 or 500-439, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 430-465 or 500-439, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 430-465 or 500-439, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 430-465 or 500-439, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 506-557.

In some embodiments, the amino acid sequence of the hIL-10R binding fusion protein comprises the amino acid sequence set forth in any one of SEQ ID NOS: 506-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-557, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 506-557.

In some embodiments, the amino acid sequence of the hIL-10R binding fusion protein consists of the amino acid sequence set forth in any one of SEQ ID NOS: 506-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-557, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 506-521.

In some embodiments, the amino acid sequence of the hIL-10R binding fusion protein comprises the amino acid sequence set forth in any one of SEQ ID NOS: 506-521, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-521, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-521, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-521, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 506-521, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 506-521.

In some embodiments, the amino acid sequence of the hIL-10R binding fusion protein consists of the amino acid sequence set forth in any one of SEQ ID NOS: 506-521, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-521, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-521, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-521, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 506-521, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 522-534.

In some embodiments, the amino acid sequence of the hIL-10R binding fusion protein comprises the amino acid sequence set forth in any one of SEQ ID NOS: 522-534, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 522-534, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 522-534, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 522-534, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 522-534, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 522-534.

In some embodiments, the amino acid sequence of the hIL-10R binding fusion protein consists of the amino acid sequence set forth in any one of SEQ ID NOS: 522-534, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 522-534, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 522-534, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 522-534, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 522-534, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 535-546.

In some embodiments, the amino acid sequence of the hIL-10R binding fusion protein comprises the amino acid sequence set forth in any one of SEQ ID NOS: 535-546, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 535-546, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 535-546, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 535-546, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 535-546, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 535-546.

In some embodiments, the amino acid sequence of the hIL-10R binding fusion protein consists of the amino acid sequence set forth in any one of SEQ ID NOS: 535-546, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 535-546, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 535-546, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 535-546, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 535-546, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 547-557.

In some embodiments, the amino acid sequence of the hIL-10R binding fusion protein comprises the amino acid sequence set forth in any one of SEQ ID NOS: 547-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 547-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 547-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 547-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein comprises the amino acid sequence set forth in any one of SEQ ID NOS: 547-557, and further comprises no more than about 1, 2, 3, 4, 5, 6, 7, 8, 9, or 10 amino acid variations (e.g., substitutions, additions, deletions, etc. (e.g., substitutions)).

In some embodiments, the amino acid sequence of the hIL-10R binding fusion protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 547-557.

In some embodiments, the amino acid sequence of the hIL-10R binding fusion protein consists of the amino acid sequence set forth in any one of SEQ ID NOS: 547-557, and further comprises 1 or more but less than 15% (less than 12%, less than 10%, less than 8%), amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 547-557, and further comprises at least about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 547-557, and further comprises about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 547-557, and further consists of about 1, 2, 3, 4, 5, 6, 7, 8, 9, or 10 amino acid variations (e.g., substitutions, additions, deletions, etc. (e.g., substitutions)). In some embodiments, the amino acid sequence of the hIL-10R binding fusion protein consists of the amino acid sequence set forth in any one of SEQ ID NOS: 547-557, and further comprises no more than about 1, 2, 3, 4, 5, 6, 7, 8, 9, or 10 amino acid variations (e.g., substitutions, additions, deletions, etc. (e.g., substitutions)).

In some embodiments, a hIL-10R binding fusion protein described herein is compared to a reference hIL-10 Fc fusion protein in the same format. The amino acid sequence of exemplary reference hIL-10-Fc fusion proteins is provided in Table 8. A person of ordinary skill in art can readily determine an appropriate reference fusion protein as needed.

TABLE 8
The Amino Acid Sequence of Exemplary Reference hIL-10 Fc and GFP-Fc Fusion
Proteins & Polypeptides.

| | | SEQ |
|---|---|---|
| Description | Amino Acid Sequence | ID NO |
| Reference hIL-10 | MYRMQLLSCIALSLALVTNSAESKYGPPCPPCPAPEAAGGPSVELFP | 412 |
| hIg Fc fusion | PKPKDTLMISRTPEVTCVVVDVSQEDPEVQFNWYVDGVEVHNAKTKP | |
| protein | REEQFNSTYRVVSVLTVLHQDWLNGKEYKCKVSNKGLPSSIEKTISK | |
| Variant | AKGQPREPQVYTLPPSQEEMTKNQVSLTCLVKGFYPSDIAVEWESNG | |
| with signal peptide | QPENNYKTTPPVLDSDGSFFLYSRLTVDKSRWQEGNVESCSVMHEAL | |
| | HNHYTQKSLSLSLGGGGGGGGSGGGGSGGGGSMHSSALLCCLVLLTG | |
| | VRASPGQGTQSENSCTHFPGNLPNMLRDLRDAFSRVKTFFQMKDQLD | |
| | NLLLKESLLEDFKGYLGCQALSEMIQFYLEEVMPQAENQDPDIKAHV | |
| | NSLGENLKTLRLRLRRCHRFLPCENKSKAVEQVKNAFNKLQEKGIYK | |
| | AMSEFDIFINYIEAYMTMKIRN | |
| Reference hIL-10 | AESKYGPPCPPCPAPEAAGGPSVELFPPKPKDTLMISRTPEVTCVVV | 413 |
| hIg Fc fusion | DVSQEDPEVQFNWYVDGVEVHNAKTKPREEQFNSTYRVVSVLTVLHQ | |
| protein | DWLNGKEYKCKVSNKGLPSSIEKTISKAKGQPREPQVYTLPPSQEEM | |
| Variant | TKNQVSLTCLVKGFYPSDIAVEWESNGQPENNYKTTPPVLDSDGSFF | |
| without signal | LYSRLTVDKSRWQEGNVFSCSVMHEALHNHYTQKSLSLSLGGGGGGG | |
| peptide | GSGGGGSGGGGSMHSSALLCCLVLLTGVRASPGQGTQSENSCTHFPG | |
| | NLPNMLRDLRDAFSRVKTFFQMKDQLDNLLLKESLLEDFKGYLGCQA | |
| | LSEMIQFYLEEVMPQAENQDPDIKAHVNSLGENLKTLRLRLRRCHRF | |
| | LPCENKSKAVEQVKNAFNKLQEKGIYKAMSEFDIFINYIEAYMTMKI | |
| | RN | |
| Reference hIL-10 | MYRMQLLSCIALSLALVINSESKYGPPCPPCPAPEAAGGPSVELEPP | 466 |
| hIg Fc fusion | KPKDTLMISRTPEVTCVVVDVSQEDPEVQFNWYVDGVEVHNAKTKPR | |
| protein | EEQFNSTYRVVSVLTVLHQDWLNGKEYKCKVSNKGLPSSIEKTISKA | |
| with signal peptide | KGQPREPQVYTLPPSQEEMTKNQVSLTCLVKGFYPSDIAVEWESNGQ | |
| | PENNYKTTPPVLDSDGSFFLYSRLTVDKSRWQEGNVFSCSVMHEALH | |
| | NHYTQKSLSLSLGGGGGGGGSGGGGSGGGGSMHSSALLCCLVLLTGV | |
| | RASPGQGTQSENSCTHFPGNLPNMLRDLRDAFSRVKTFFQMKDQLDN | |
| | LLLKESLLEDFKGYLGCQALSEMIQFYLEEVMPQAENQDPDIKAHVN | |
| | SLGENLKTLRLRLRRCHRFLPCENKSKAVEQVKNAFNKLQEKGIYKA | |
| | MSEFDIFINYIEAYMTMKIRN | |
| Reference hIL-10 | ESKYGPPCPPCPAPEAAGGPSVFLFPPKPKDTLMISRTPEVTCVVVD | 467 |
| hIg Fc fusion | VSQEDPEVQFNWYVDGVEVHNAKTKPREEQFNSTYRVVSVLTVLHQD | |
| protein | WLNGKEYKCKVSNKGLPSSIEKTISKAKGQPREPQVYTLPPSQEEMT | |
| without signal | KNQVSLTCLVKGFYPSDIAVEWESNGQPENNYKTTPPVLDSDGSFEL | |
| peptide | YSRLTVDKSRWQEGNVFSCSVMHEALHNHYTQKSLSLSLGGGGGGGG | |
| | SGGGGSGGGGSMHSSALLCCLVLLTGVRASPGQGTQSENSCTHEPGN | |
| | LPNMLRDLRDAFSRVKTFFQMKDQLDNLLLKESLLEDFKGYLGCQAL | |
| | SEMIQFYLEEVMPQAENQDPDIKAHVNSLGENLKTLRLRLRRCHREL | |
| | PCENKSKAVEQVKNAFNKLQEKGIYKAMSEFDIFINYIEAYMTMKIR | |
| | N | |
| Reference hIL-10 | MYRMQLLSCIALSLALVTNSVPRDCGCKPCICTVPEVSSVFIFPPKP | 558 |
| mIgG1 Fc fusion | KDVLTITLTPKVTCVVVAISKDDPEVQFSWFVDDVEVHTAQTQPREE | |
| protein | QFNSTFRSVSELPIMHQDWLNGKEFKCRVNSAAFPAPIEKTISKTKG | |
| with signal | RPKAPQVYTIPPPKEQMAKDKVSLTCMITDFFPEDITVEWQWNGQPA | |
| peptide | ENYKNTQPIMDTDGSYFVYSKLNVQKSNWEAGNTFTCSVLHEGLHNH | |
| | HTEKSLSHSPGGGGGGGSGGGGSGGGGSSPGQGTQSENSCTHEPGNL | |
| | PNMLRDLRDAFSRVKTFFQMKDQLDNLLLKESLLEDFKGYLGCQALS | |
| | EMIQFYLEEVMPQAENQDPDIKAHVNSLGENLKTLRLRLRRCHRELP | |
| | CENKSKAVEQVKNAFNKLQEKGIYKAMSEFDIFINYIEAYMTMKIRN | |
| Reference hIL-10 | VPRDCGCKPCICTVPEVSSVFIFPPKPKDVLTITLTPKVTCVVVAIS | 559 |
| mIgG1 Fc fusion | KDDPEVQFSWFVDDVEVHTAQTQPREEQFENSTERSVSELPIMHQDWL | |
| protein | NGKEFKCRVNSAAFPAPIEKTISKTKGRPKAPQVYTIPPPKEQMAKD | |
| without signal | KVSLTCMITDFFPEDITVEWQWNGQPAENYKNTQPIMDTDGSYFVYS | |
| peptide | KLNVQKSNWEAGNTFTCSVLHEGLHNHHTEKSLSHSPGGGGGGGSGG | |
| | GGSGGGGSSPGQGTQSENSCTHFPGNLPNMLRDLRDAFSRVKTFFQM | |
| | KDQLDNLLLKESLLEDFKGYLGCQALSEMIQFYLEEVMPQAENQDPD | |
| | IKAHVNSLGENLKTLRLRLRRCHRFLPCENKSKAVEQVKNAFNKLQE | |
| | KGIYKAMSEFDIFINYIEAYMTMKIRN | |
| Reference hIL-10 | MYRMQLLSCIALSLALVINSEPRGPTIKPCLLCKCPAPNAAGGPSVE | 560 |
| mIgG2a Fc fusion | IFPPKIKDVLMISLSPIVTCVVVDVSEDDPDVQISWFVNNVEVHTAQ | |
| protein | TQTHREDYNSTLRVVSALPIQHQDWMSGKEFKCKVNNKDLPAPIERT | |
| with signal | ISKPKGSVRAPQVYVLPPPEEEMTKKQVTLTCMVTDEMPEDIYVEWT | |
| peptide | NNGKTELNYKNTEPVLDSDGSYFMYSKLRVEKKNWVERNSYSCSVVH | |
| | EGLHNHHTTKSFSRTPGKGGGGGGGSGGGGSGGGGSSPGQGTQSENS | |
| | CTHFPGNLPNMLRDLRDAFSRVKTFFQMKDQLDNLLLKESLLEDEKG | |
| | YLGCQALSEMIQFYLEEVMPQAENQDPDIKAHVNSLGENLKTLRLRL | |
| | RRCHRFLPCENKSKAVEQVKNAFNKLQEKGIYKAMSEFDIFINYIEA | |
| | YMTMKIRN | |
| Reference hIL-10 | EPRGPTIKPCLLCKCPAPNAAGGPSVFIFPPKIKDVLMISLSPIVTC | 561 |
| mIgG2a Fc fusion | VVVDVSEDDPDVQISWFVNNVEVHTAQTQTHREDYNSTLRVVSALPI | |
| protein | QHQDWMSGKEFKCKVNNKDLPAPIERTISKPKGSVRAPQVYVLPPPE | |
| without signal | EEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGKGG | |
| | GGGGGSGGGGSGGGGSSPGQGTQSENSCTHFPGNLPNMLRDLRDAES | |
| | RVKTFFQMKDQLDNLLLKESLLEDFKGYLGCQALSEMIQFYLEEVMP | |
| | QAENQDPDIKAHVNSLGENLKTLRLRLRRCHRFLPCENKSKAVEQVK | |
| | NAFNKLQEKGIYKAMSEFDIFINYIEAYMTMKIRN | |
| Reference hIL-10 | MYRMQLLSCIALSLALVTNSEPRGPTIKPCPPCKCPAPNAAGGPSVE | 562 |
| mIgG2a Fc fusion | IFPPKIKDVLMISLSPIVTCVVVDVSEDDPDVQISWFVNNVEVHTAQ | |
| protein | TQTHREDYNSTLRVVSALPIQHQDWMSGKEFKCKVNNKDLGAPIERT | |
| Variant Fc | ISKPKGSVRAPQVYVLPPPEEEMTKKQVTLTCMVTDEMPEDIYVEWT | |
| with signal peptide | NNGKTELNYKNTEPVLDSDGSYFMYSKLRVEKKNWVERNSYSCSVVH | |
| | EGLHNHHTTKSFSRTPGKGGGGGGGSGGGGSGGGGSSPGQGTQSENS | |
| | CTHFPGNLPNMLRDLRDAFSRVKTFFQMKDQLDNLLLKESLLEDFKG | |
| | YLGCQALSEMIQFYLEEVMPQAENQDPDIKAHVNSLGENLKTLRLRL | |
| | RRCHRFLPCENKSKAVEQVKNAFNKLQEKGIYKAMSEFDIFINYIEA | |
| | YMTMKIRN | |
| Reference hIL-10 | EPRGPTIKPCPPCKCPAPNAAGGPSVFIFPPKIKDVLMISLSPIVTC | 563 |
| mIgG2a Fc fusion | VVVDVSEDDPDVQISWFVNNVEVHTAQTQTHREDYNSTLRVVSALPI | |
| protein | QHQDWMSGKEFKCKVNNKDLGAPIERTISKPKGSVRAPQVYVLPPPE | |
| Variant Fc | EEMTKKQVTLTCMVTDEMPEDIYVEWTNNGKTELNYKNTEPVLDSDG | |
| without signal | SYFMYSKLRVEKKNWVERNSYSCSVVHEGLHNHHTTKSFSRTPGKGG | |
| peptide | GGGGGSGGGGSGGGGSSPGQGTQSENSCTHEPGNLPNMLRDLRDAFS | |
| | RVKTFFQMKDQLDNLLLKESLLEDFKGYLGCQALSEMIQFYLEEVMP | |
| | QAENQDPDIKAHVNSLGENLKTLRLRLRRCHRFLPCENKSKAVEQVK | |
| | NAFNKLQEKGIYKAMSEFDIFINYIEAYMTMKIRN | |
| Reference mIgG2a | MYRMQLLSCIALSLALVINSEPRGPTIKPCPPCKCPAPNAAGGPSVE | 564 |
| Fc fusion protein | IFPPKIKDVLMISLSPIVTCVVVDVSEDDPDVQISWFVNNVEVHTAQ | |
| Variant Fc | TQTHREDYNSTLRVVSALPIQHQDWMSGKEFKCKVNNKDLGAPIERT | |
| with signal peptide | ISKPKGSVRAPQVYVLPPPEEEMTKKQVTLTCMVTDEMPEDIYVEWT | |
| | NNGKTELNYKNTEPVLDSDGSYFMYSKLRVEKKNWVERNSYSCSVVH | |
| | EGLHNHHTTKSFSRTPGK | |
| Reference mIgG2a | EPRGPTIKPCPPCKCPAPNAAGGPSVFIFPPKIKDVLMISLSPIVTC | 565 |
| Fc fusion protein | VVVDVSEDDPDVQISWFVNNVEVHTAQTQTHREDYNSTLRVVSALPI | |
| Variant Fc | QHQDWMSGKEFKCKVNNKDLGAPIERTISKPKGSVRAPQVYVLPPPE | |
| without signal | EEMTKKQVTLTCMVTDEMPEDIYVEWTNNGKTELNYKNTEPVLDSDG | |
| peptide | SYFMYSKLRVEKKNWVERNSYSCSVVHEGLHNHHTTKSFSRTPGK | |
| Reference mIgG1 | MYRMQLLSCIALSLALVTNSVPRDCGCKPCICTVPEVSSVFIFPPKP | 566 |
| Fc fusion protein | KDVLTITLTPKVTCVVVAISKDDPEVQFSWFVDDVEVHTAQTQPREE | |
| with signal peptide | QFNSTFRSVSELPIMHQDWLNGKEFKCRVNSAAFPAPIEKTISKTKG | |
| | RPKAPQVYTIPPPKEQMAKDKVSLTCMITDFFPEDITVEWQWNGQPA | |
| | ENYKNTQPIMDTDGSYFVYSKLNVQKSNWEAGNTFTCSVLHEGLHNH | |
| | HTEKSLSHSP | |
| Reference mIgG1 | VPRDCGCKPCICTVPEVSSVFIFPPKPKDVLTITLTPKVTCVVVAIS | 567 |
| Fc fusion protein | KDDPEVQFSWFVDDVEVHTAQTQPREEQENSTERSVSELPIMHQDWL | |
| without signal | NGKEFKCRVNSAAFPAPIEKTISKTKGRPKAPQVYTIPPPKEQMAKD | |
| peptide | KVSLTCMITDFFPEDITVEWQWNGQPAENYKNTQPIMDTDGSYFVYS | |
| | KLNVQKSNWEAGNTFTCSVLHEGLHNHHTEKSLSHSP | |
| Reference hIgG4 | MYRMQLLSCIALSLALVTNSAESKYGPPCPPCPAPEAAGGPSVELEP | 568 |
| Fc fusion protein | PKPKDTLMISRTPEVTCVVVDVSQEDPEVQFNWYVDGVEVHNAKTKP | |
| Variant Fc | REEQFNSTYRVVSVLTVLHQDWLNGKEYKCKVSNKGLPSSIEKTISK | |
| with signal peptide | AKGQPREPQVYTLPPSQEEMTKNQVSLTCLVKGFYPSDIAVEWESNG | |
| | QPENNYKTTPPVLDSDGSFFLYSRLTVDKSRWQEGNVESCSVMHEAL | |
| | HNHYTQKSLSLSL | |
| Reference hIgG4 | AESKYGPPCPPCPAPEAAGGPSVELFPPKPKDTLMISRTPEVTCVVV | 569 |
| Fc fusion protein | DVSQEDPEVQFNWYVDGVEVHNAKTKPREEQFNSTYRVVSVLTVLHQ | |
| Variant Fc | DWLNGKEYKCKVSNKGLPSSIEKTISKAKGQPREPQVYTLPPSQEEM | |
| without signal | TKNQVSLTCLVKGFYPSDIAVEWESNGQPENNYKTTPPVLDSDGSFF | |
| peptide | LYSRLTVDKSRWQEGNVFSCSVMHEALHNHYTQKSLSLSL | |

In some embodiments, the amino acid sequence of the reference hIL-10 Fc fusion protein comprises or consists of the amino acid sequence of a polypeptide set forth in Table 8. In some embodiments, the amino acid sequence of the reference hIL-10 Fc fusion protein comprises or consists of the amino acid sequence set forth in SEQ ID NO: 412. In some embodiments, the amino acid sequence of reference hIL-10 Fc fusion protein comprises or consists of the amino acid sequence set forth in SEQ ID NO: 413. In some embodiments, the amino acid sequence of reference hIL-10 Fc fusion protein comprises or consists of the amino acid sequence set forth in SEQ ID NO: 466. In some embodiments, the amino acid sequence of reference hIL-10 Fc fusion protein comprises or consists of the amino acid sequence set forth in SEQ ID NO: 467. In some embodiments, the amino acid sequence of reference hIL-10 Fc fusion protein comprises or consists of the amino acid sequence set forth in SEQ ID NO: 558. In some embodiments, the amino acid sequence of reference hIL-10 Fc fusion protein comprises or consists of the amino acid sequence set forth in SEQ ID NO: 559. In some embodiments, the amino acid sequence of reference hIL-10 Fc fusion protein comprises or consists of the amino acid sequence set forth in SEQ ID NO: 560. In some embodiments, the amino acid sequence of reference hIL-10 Fc fusion protein comprises or consists of the amino acid sequence set forth in SEQ ID NO: 561. In some embodiments, the amino acid sequence of reference hIL-10 Fc fusion protein comprises or consists of the amino acid sequence set forth in SEQ ID NO: 562. In some embodiments, the amino acid sequence of reference hIL-10 Fc fusion protein comprises or consists of the amino acid sequence set forth in SEQ ID NO: 563. In some embodiments, the amino acid sequence of reference hIL-10 Fc fusion protein comprises or consists of the amino acid sequence set forth in SEQ ID NO: 564. In some embodiments, the amino acid sequence of reference hIL-10 Fc fusion protein comprises or consists of the amino acid sequence set forth in SEQ ID NO: 565. In some embodiments, the amino acid sequence of reference hIL-10 Fc fusion protein comprises or consists of the amino acid sequence set forth in SEQ ID NO: 566. In some embodiments, the amino acid sequence of reference hIL-10 Fc fusion protein comprises or consists of the amino acid sequence set forth in SEQ ID NO: 567. In some embodiments, the amino acid sequence of reference hIL-10 Fc fusion protein comprises or consists of the amino acid sequence set forth in SEQ ID NO: 568. In some embodiments, the amino acid sequence of reference hIL-10 Fc fusion protein comprises or consists of the amino acid sequence set forth in SEQ ID NO: 569.

### 5.11 Polycistronic Nucleic Acid Molecules

Further provided herein are nucleic acid molecules comprising a coding region encoding a hIL-10R binding protein (or functional fragment and/or functional variant thereof) (e.g., described herein, see, e.g., § 5.2) and a coding region encoding a first immunogenic protein (or immunogenic fragment and/or immunogenic variant thereof) (e.g., described herein, see, e.g., § 5.5).

In some embodiments, the nucleic acid molecule comprises a nucleic acid molecule described in § 5.4. In some embodiments, the nucleic acid molecule comprises a nucleic acid molecule described in § 5.6. In some embodiments, the nucleic acid molecule comprises a nucleic acid molecule described in § 5.4 and a nucleic acid molecule described in § 5.6.

In some embodiments, the nucleic acid molecule encoding the hIL-10R binding protein (or the functional fragment and/or functional variant thereof) (e.g., described herein, see, e.g., § 5.2) comprises a nucleic acid molecule described in § 5.4. In some embodiments, the nucleic acid molecule encoding the first immunogenic protein (or immunogenic fragment and/or immunogenic variant thereof) (e.g., described herein, see, e.g., § 5.5) comprises a nucleic acid molecule described in § 5.6. In some embodiments, the nucleic acid molecule encoding the hIL-10R binding protein (or the functional fragment and/or functional variant thereof) (e.g., described herein, see, e.g., § 5.2) comprises a nucleic acid molecule described in § 5.4; and the nucleic acid molecule encoding the first immunogenic protein (or immunogenic fragment and/or immunogenic variant thereof) (e.g., described herein, see, e.g., § 5.5) comprises a nucleic acid molecule described in § 5.6.

In some embodiments, the encoded hIL-10R binding protein (or the functional fragment and/or functional variant thereof) comprises a hIL-10R binding protein (or a functional fragment and/or functional variant thereof) described in § 5.2. In some embodiments, the encoded first immunogenic protein (or immunogenic fragment and/or immunogenic variant thereof) comprises an immunogenic protein (or immunogenic fragment and/or immunogenic variant thereof) described in § 5.5. In some embodiments, the encoded hIL-10R binding protein (or the functional fragment and/or functional variant thereof) comprises a hIL-10R binding protein (or a functional fragment and/or functional variant thereof) described in § 5.2; and the encoded first immunogenic protein (or immunogenic fragment and/or immunogenic variant thereof) comprises an immunogenic protein (or immunogenic fragment and/or immunogenic variant thereof) described in § 5.5.

In some embodiments, the nucleic acid molecule further comprises a coding region encoding an IGIP (e.g., hIGIP) protein (or a functional fragment and/or functional variant thereof) (e.g., described herein, see, e.g., § 5.7). In some embodiments, the encoded IGIP (e.g., hIGIP) protein (or the functional fragment and/or functional variant thereof) comprises an IGIP (e.g., hIGIP) protein (or a functional fragment and/or functional variant thereof) described in § 5.7. In some embodiments, the nucleic acid molecule encoding the IGIP (e.g., hIGIP) protein (or the functional fragment and/or functional variant thereof) (e.g., described herein, see, e.g., § 5.7) comprises a nucleic acid molecule described in § 5.8.

In some embodiments, the nucleic acid molecule is a DNA molecule. In some embodiments, the nucleic acid molecule is an RNA molecule (e.g., mRNA or circular RNA). In some embodiments, the nucleic acid molecule is an mRNA. In some embodiments, the nucleic acid molecule is a circular RNA molecule.

In some embodiments, the nucleic acid molecule is a linear coding nucleic acid construct. In some embodiments, the nucleic acid molecule is comprised within one or more vectors (e.g., vectors described herein (see, e.g., § 5.14)). In some embodiments, the nucleic acid molecule is contained within a vector non-viral vector (e.g., a plasmid), viral vector). In some embodiments, the nucleic acid molecule is contained within a non-viral vector. In some embodiments, the nucleic acid molecule is contained within a plasmid. In some embodiments, the nucleic acid molecule is contained within a viral vector. A more detailed description of vectors (e.g., non-viral (e.g., plasmids) and viral) for both RNA and DNA nucleic acids is provided in § 5.14. In some embodiments, the nucleic acid molecule or the vector is formulated in one or more carrier (e.g., a carrier described herein (see, e.g., § 5.15)).

The nucleic acid molecules can be generated using common methods known in the art and described above in § 5.17. Methods are known in the art to express multiple proteins from a single nucleic acid molecule. For example, additional elements may be included within the nucleic acid molecule to efficiently transcribe (DNA molecules) or translate (RNA molecules, e.g., mRNA molecules) multiple coding sequences within the same nucleic acid molecule. See, e.g., de Felipe P. (2002). Polycistronic viral vectors. Current gene therapy, 2(3), 355-378. https://doi.org/10.2174/1566523023347742, the entire contents of which is incorporated by reference herein for all purposes. For example, in the case of mRNA nucleic acids, additional elements such as internal ribosome entry sites (IRESs) or self-cleaving 2A peptides may be utilized. See, e.g., Chan H Y, V S, Xing X, et al. Comparison of IRES and F2A-based locus-specific multicistronic expression in stable mouse lines. PLoS One. 2011; 6(12): e28885. doi:10.1371/journal.pone.0028885, the entire contents of which is incorporated by reference herein for all purposes.

### 5.11.1 Plurality of Immunogens

In some embodiments, the nucleic acid molecule comprises a plurality of coding regions each encoding an immunogenic protein (or immunogenic fragments and/or immunogenic variants thereof) (e.g., described herein, see, e.g., § 5.5)).

In some embodiments, the plurality comprises at least 2, 3, 4, 5, 6, 7, 8, 9, 10, 11, 12, 13, 14, 15, 16, 17, 18, 19, 20, 30, 40, 50, 60, 70, 80, 90, 100 or more coding regions. In some embodiments, the plurality comprises from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 coding regions. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 coding regions. In some embodiments, the plurality comprises 2, 3, 4, 5, 6, 7, 8, 9, 10, 11, 12, 13, 14, 15, 16, 17, 18, 19, 20, 30, 40, 50, 60, 70, 80, 90, 100 or more coding regions. In some embodiments, the plurality consists of 2, 3, 4, 5, 6, 7, 8, 9, 10, 11, 12, 13, 14, 15, 16, 17, 18, 19, 20, 30, 40, 50, 60, 70, 80, 90, 100 or more coding regions.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the encoded immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each encoded immunogenic (or immunogenic fragments and/or immunogenic variants thereof) of the plurality is different. In some embodiments, each encoded immunogenic protein (or immunogenic fragments and/or immunogenic variants thereof) of the plurality comprises a different amino acid sequence relative to the amino acid sequence of the other members of the plurality. In some embodiments, the amino acid sequence of each encoded immunogenic protein (or immunogenic fragments and/or immunogenic variants thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different organisms (e.g., different viruses). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from the same organisms (e.g., the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different strains of the same organism (e.g., different strains of the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different variant of the same immunogenic protein.

In some embodiments, at least two of the encoded immunogenic proteins (or immunogenic fragments and/or immunogenic variants thereof) of the plurality are from different organisms (e.g., different viruses). In some embodiments, at least two of the encoded immunogenic proteins (or immunogenic fragments and/or immunogenic variants thereof) of the plurality are from the same organisms (e.g., the same virus). In some embodiments, at least two of the encoded immunogenic proteins (or immunogenic fragments and/or immunogenic variants thereof) of the plurality are from different strains of the same organism (e.g., different strains of the same virus). In some embodiments, each encoded immunogenic protein of the plurality comprises a different variant of the same immunogenic protein.

In some embodiments, at least one of the encoded immunogenic proteins (or immunogenic fragment and/or immunogenic variant thereof) of the plurality is a first virus respiratory virus immunogenic protein (or immunogenic fragment and/or immunogenic variant thereof); and at least one of the encoded immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are variants of the same tumor associated immunogen.

### 5.12 Combination Compositions

Further provided herein are compositions (e.g., vaccine compositions (e.g., vaccine prime and vaccine booster compositions), pharmaceutical compositions) comprising a hIL-10R binding agent (e.g., a hIL-10R binding protein (or a functional fragment and/or functional variant thereof) (e.g., described herein, see, e.g., § 5.2) (or a fusion protein or conjugate thereof) or a nucleic acid molecule encoding a hIL-10R binding protein (or a functional fragment and/or functional variant thereof)) (e.g., described herein, see, e.g., § 5.4)) (or a fusion protein or conjugate thereof); and at least one immunogen (e.g., immunogenic protein (or immunogenic fragment and/or immunogenic variant thereof)) (e.g., described herein, see, e.g., § 5.5) (or a nucleic acid molecule encoding the immunogenic protein (or immunogenic fragment and/or immunogenic variant thereof) (e.g., described herein, see, e.g., § 5.6)).

Further provided herein are compositions (e.g., vaccine compositions (e.g., vaccine prime and vaccine booster compositions), pharmaceutical compositions) comprising a hIL-10R binding agent (e.g., a hIL-10R binding protein (or a functional fragment and/or functional variant thereof) (e.g., described herein, see, e.g., § 5.2) (or a fusion protein or conjugate thereof) or a nucleic acid molecule encoding a hIL-10R binding protein (or a functional fragment and/or functional variant thereof)) (e.g., described herein, see, e.g., § 5.4)) (or a fusion protein or conjugate thereof); and an IGIP (e.g., hIGIP) protein (or a functional fragment and/or functional variant thereof) (e.g., described herein, see, e.g., § 5.7) (or a fusion protein or conjugate thereof) or a nucleic acid molecule encoding an IGIP (e.g., hIGIP) protein (or a functional fragment and/or functional variant thereof) (e.g., described herein, see, e.g., § 5.8).

Further provided herein are compositions (e.g., vaccine compositions (e.g., vaccine prime and vaccine booster compositions), pharmaceutical compositions) comprising a hIL-10R binding agent (e.g., a hIL-10R binding protein (or a functional fragment and/or functional variant thereof) (e.g., described herein, see, e.g., § 5.2) (or a fusion protein or conjugate thereof) or a nucleic acid molecule encoding a hIL-10R binding protein (or a functional fragment and/or functional variant thereof)) (e.g., described herein, see, e.g., § 5.4)) (or a fusion protein or conjugate thereof); an IGIP (e.g., hIGIP) protein (or a functional fragment and/or functional variant thereof) (e.g., described herein, see, e.g., § 5.7) (or a fusion protein or conjugate thereof) or a nucleic acid molecule encoding an IGIP (e.g., hIGIP) protein (or a functional fragment and/or functional variant thereof) (e.g., described herein, see, e.g., § 5.8); and at least one immunogen (e.g., immunogenic protein (or immunogenic fragment and/or immunogenic variant thereof)) (e.g., described herein, see, e.g., § 5.5) (or a nucleic acid molecule encoding the immunogenic protein (or immunogenic fragment and/or immunogenic variant thereof) (e.g., described herein, see, e.g., § 5.6)).

In some embodiments, the composition is less reactogenic when administered to a subject (e.g., a human subject) relative to a control composition that does not contain the hIL-10R binding agent (e.g., a hIL-10R binding protein (or a functional fragment and/or functional variant thereof) (e.g., described herein, see, e.g., § 5.2) (or a fusion protein or conjugate thereof) or a nucleic acid molecule encoding a hIL-10R binding protein (or a functional fragment and/or functional variant thereof)) (e.g., described herein, see, e.g., § 5.4)) (or a fusion protein or conjugate thereof).

In some embodiments, the composition decreases the level of a proinflammatory cytokine (e.g., a proinflammatory cytokine associated with reactogenicity (e.g., IL-1β, IFNγ, IL-6, etc.)) by at least about 5%, 10%, 15%, 20%, 25%, 30%, 35%, 40%, 45%, 50%, 55%, 60%, 65%, 70%, 75%, 80%, 85%, 90%, or 95% or more when administered to a subject (e.g., a human subject) relative to a control composition that does not contain the hIL-10R binding agent (e.g., a hIL-10R binding protein (or a functional fragment and/or functional variant thereof). In some embodiments, the composition decreases the level of a proinflammatory cytokine (e.g., a proinflammatory cytokine associated with reactogenicity (e.g., IL-1β, IFNγ, IL-6, etc.)) from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% when administered to a subject (e.g., a human subject) relative to a control composition that does not contain the hIL-10R binding agent (e.g., a hIL-10R binding protein (or a functional fragment and/or functional variant thereof).

Exemplary pro-inflammatory cytokines include those associated with reactogenicity. Exemplary pro-inflammatory cytokines include, e.g., IL-1β, IFNγ, IL-6, and TNF-α. In some embodiments, the pro-inflammatory cytokine is IL-1β. In some embodiments, the pro-inflammatory cytokine is IFNγ. In some embodiments, the pro-inflammatory cytokine is IL-6. In some embodiments, the pro-inflammatory cytokine is TNF-α.

### 5.12.1 Nucleic Acid-Based Compositions

In some embodiments, the composition (e.g., vaccine compositions (e.g., vaccine prime and vaccine booster compositions), pharmaceutical compositions) comprises a nucleic acid molecule comprising a coding region encoding a hIL-10R binding protein (or functional fragment and/or functional variant thereof) (e.g., described herein, see, e.g., § 5.4)); and a nucleic acid molecule comprising a coding region encoding an immunogenic protein (or immunogenic fragment and/or immunogenic variant thereof) (e.g., described herein, see, e.g., § 5.6)).

In some embodiments, the nucleic acid molecule comprising a coding region encoding a hIL-10R binding protein (or functional fragment and/or functional variant thereof) is a nucleic acid molecule described in § 5.4. In some embodiments, the nucleic acid molecule comprising a coding region encoding the immunogenic protein (or immunogenic fragment and/or immunogenic variant thereof) is a nucleic acid molecule described in § 5.6. In some embodiments, the nucleic acid molecule comprising a coding region encoding a hIL-10R binding protein (or functional fragment and/or functional variant thereof) is a nucleic acid molecule described in § 5.4; and the nucleic acid molecule comprising a coding region encoding the immunogenic protein (or immunogenic fragment and/or immunogenic variant thereof) is a nucleic acid molecule described in § 5.6.

In some embodiments, the encoded hIL-10R binding protein (or functional fragment and/or functional variant thereof) is a hIL-10R binding protein (or a functional fragment and/or functional variant thereof) described in § 5.2. In some embodiments, the encoded immunogenic protein (or immunogenic fragment and/or immunogenic variant thereof) is an immunogenic protein (or immunogenic fragment and/or immunogenic variant thereof) described in § 5.5. In some embodiments, the encoded hIL-10R binding protein (or functional fragment and/or functional variant thereof) is a hIL-10R binding protein (or a functional fragment and/or functional variant thereof) described in § 5.2; and the encoded immunogenic protein (or immunogenic fragment and/or immunogenic variant thereof) is an immunogenic protein (or immunogenic fragment and/or immunogenic variant thereof) described in § 5.5.

The nucleic acid molecule encoding the hIL-10R binding protein (or functional fragment and/or functional variant thereof) (e.g., described herein) and the nucleic acid molecule encoding the immunogenic protein (or immunogenic fragment and/or immunogenic variant thereof) (e.g., described herein) can be part of the same larger nucleic acid molecule or separate (i.e., not connected) nucleic acid molecules. In some embodiments, the nucleic acid molecule encoding the hIL-10R binding protein (or functional fragment and/or functional variant thereof) (e.g., described herein) and the nucleic acid molecule encoding the immunogenic protein (or immunogenic fragment and/or immunogenic variant thereof) (e.g., described herein) are part of the same nucleic acid molecule. In some embodiments, the nucleic acid molecule encoding the hIL-10R binding protein (or functional fragment and/or functional variant thereof) (e.g., described herein) and the nucleic acid molecule encoding the immunogenic protein (or immunogenic fragment and/or immunogenic variant thereof) (e.g., described herein) are not part of the same nucleic acid molecule. In some embodiments, the nucleic acid molecule encoding the hIL-10R binding protein (or functional fragment and/or functional variant thereof) (e.g., described herein) and the nucleic acid molecule encoding the immunogenic protein (or immunogenic fragment and/or immunogenic variant thereof) (e.g., described herein) are separate (i.e., not connected) nucleic acid molecules.

In some embodiments, the composition (e.g., vaccine booster composition, pharmaceutical composition) comprises a nucleic acid molecule comprising a coding region encoding an IGIP (e.g., hIGIP) protein (or a functional fragment and/or functional variant thereof) (e.g., described herein, see, e.g., § 5.7). In some embodiments, the composition (e.g., vaccine booster composition, pharmaceutical composition) comprises a nucleic acid molecule comprising a coding region encoding an IGIP (e.g., hIGIP) protein (or a functional fragment and/or functional variant thereof) described in § 5.8. The nucleic acid molecule encoding the IGIP (e.g., hIGIP) protein (or a functional fragment and/or functional variant thereof) can be part of the can be part of the same larger nucleic acid molecule or separate (i.e., not connected) nucleic acid molecules as those encoding the hIL-10R binding protein (or functional fragment and/or functional variant thereof) (e.g., described herein) and/or the immunogenic protein (or immunogenic fragment and/or immunogenic variant thereof) (e.g., described herein).

### 5.12.1.1 Plurality of Immunogens

In some embodiments, the composition (e.g., vaccine compositions (e.g., vaccine prime and vaccine booster compositions), pharmaceutical compositions) comprises a plurality of nucleic acid molecules each comprising a coding region encoding an immunogenic protein (or immunogenic fragment and/or immunogenic variant thereof) (e.g., described herein, see, e.g., § 5.6)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more nucleic acid molecules. In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleic acid molecules. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 nucleic acid molecules.

In some embodiments, each of the nucleic acid molecules of the plurality are part of the same larger nucleic acid molecule. In some embodiments, each of the nucleic acid molecules of the plurality are separate (i.e., not connected) nucleic acid molecules. In some embodiments, at least two of the nucleic acid molecules of the plurality are part of the same larger nucleic acid molecule. In some embodiments, at least two of the nucleic acid molecules of the plurality are separate (i.e., not connected) nucleic acid molecules. In some embodiments, at least two of the nucleic acid molecules of the plurality are part of the same larger nucleic acid molecule; and at least one (e.g., at least 2, 3, 4, 5, etc.) of the nucleic acid molecules of the plurality is a separate (i.e., not connected) nucleic acid molecule.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the encoded immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each encoded immunogenic protein (or immunogenic fragment and/or immunogenic variant thereof) of the plurality is different. In some embodiments, each encoded immunogenic protein (or immunogenic fragment and/or immunogenic variant thereof) of the plurality comprises a different amino acid sequence relative to the amino acid sequence of the other members of the plurality. In some embodiments, the amino acid sequence of each encoded immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different organisms (e.g., different viruses). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from the same organisms (e.g., the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different strains of the same organism (e.g., different strains of the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different variant of the same immunogenic protein.

In some embodiments, at least two of the encoded immunogenic proteins (or immunogenic fragments and/or immunogenic variants thereof) of the plurality are from different organisms (e.g., different viruses). In some embodiments, at least two of the encoded immunogenic proteins (or immunogenic fragments and/or immunogenic variants thereof) of the plurality are from the same organisms (e.g., the same virus). In some embodiments, at least two of the encoded immunogenic proteins (or immunogenic fragments and/or immunogenic variants thereof) of the plurality are from different strains of the same organism (e.g., different strains of the same virus). In some embodiments, each encoded immunogenic protein of the plurality comprises a different variant of the same immunogenic protein.

In some embodiments, at least one of the encoded immunogenic proteins (or immunogenic fragment and/or immunogenic variant thereof) of the plurality is a first virus respiratory virus immunogenic protein (or immunogenic fragment and/or immunogenic variant thereof); and at least one of the encoded immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are variants of the same tumor associated immunogen.

### 5.12.2 Protein-Based Compositions

In some embodiments, the composition (e.g., vaccine compositions (e.g., vaccine prime and vaccine booster compositions), pharmaceutical compositions) comprises a hIL-10R binding protein (or functional fragment and/or functional variant thereof) (e.g., described herein, see, e.g., § 5.2); and an immunogenic protein (or immunogenic fragment and/or immunogenic variant thereof) (e.g., described herein, see, e.g., § 5.5).

In some embodiments, the hIL-10R binding protein (or functional fragment and/or functional variant thereof) is a hIL-10R binding protein (or functional fragment and/or functional variant thereof) described in § 5.2. In some embodiments, the immunogenic protein (or immunogenic fragment and/or immunogenic variant thereof) is an immunogenic protein (or immunogenic fragment and/or immunogenic variant thereof) described in § 5.5. In some embodiments, the hIL-10R binding protein (or functional fragment and/or functional variant thereof) is a hIL-10R binding protein (or functional fragment and/or functional variant thereof) described in § 5.2; and the immunogenic protein (or immunogenic fragment and/or immunogenic variant thereof) is an immunogenic protein (or immunogenic fragment and/or immunogenic variant thereof) described in § 5.5.

In some embodiments, the hIL-10R binding protein (or functional fragment and/or functional variant thereof) is encoded by a nucleic acid molecule described in § 5.4. In some embodiments, the immunogenic protein (or immunogenic fragment and/or immunogenic variant thereof) is encoded by a nucleic acid molecule described in § 5.6. In some embodiments, the hIL-10R binding protein (or functional fragment and/or functional variant thereof) is encoded by a nucleic acid molecule described in § 5.4; and the immunogenic protein (or immunogenic fragment and/or immunogenic variant thereof) is encoded by a nucleic acid molecule described in § 5.6.

In some embodiments, the composition (e.g., vaccine compositions (e.g., vaccine prime and vaccine booster compositions), pharmaceutical compositions) comprises an IGIP (e.g., hIGIP) protein (or a functional fragment and/or functional variant thereof) (e.g., described herein, see, e.g., § 5.7). In some embodiments, the IGIP (e.g., hIGIP) protein (or a functional fragment and/or functional variant thereof) is described in § 5.7._In some embodiments, the IGIP (e.g., hIGIP) protein (or a functional fragment and/or functional variant thereof) is encoded by a nucleic acid molecule described in § 5.8.

### 5.12.2.1 Plurality of Immunogens

In some embodiments, the composition (e.g., vaccine compositions (e.g., vaccine prime and vaccine booster compositions), pharmaceutical compositions) comprises a plurality of immunogenic proteins (or immunogenic fragments and/or immunogenic variants thereof) (e.g., described herein, see, e.g., § 5.5)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more immunogenic proteins (or immunogenic fragments and/or immunogenic variants thereof). In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 immunogenic proteins (or immunogenic fragments and/or immunogenic variants thereof). In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 immunogenic proteins (or immunogenic fragments and/or immunogenic variants thereof).

In some embodiments, at least two (e.g., at least 2, 3, 4, 5, 6, 7, 8, 9, 10, or more) of the immunogenic proteins (or immunogenic fragments and/or immunogenic variants thereof) of the plurality are different. In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each immunogenic protein (or immunogenic fragment and/or immunogenic variant thereof) of the plurality is different. In some embodiments, each immunogenic protein (or immunogenic fragment and/or immunogenic variant thereof) of the plurality comprises a different amino acid sequence relative to the amino acid sequence of the other members of the plurality. In some embodiments, the amino acid sequence of each immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the immunogenic proteins (or immunogenic fragments and/or immunogenic variants thereof) of the plurality are from different pathogens (e.g., different viruses). In some embodiments, at least two (e.g., at least 2, 3, 4, 5, 6, 7, 8, 9, 10, or more) of the immunogenic proteins (or immunogenic fragments and/or immunogenic variants thereof) of the plurality are from the same pathogens (e.g., the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality are from different strains of the same pathogen (e.g., different strains of the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different variant of the same immunogenic protein.

In some embodiments, at least two of the immunogenic proteins (or immunogenic fragments and/or immunogenic variants thereof) of the plurality are from different pathogens (e.g., different viruses). In some embodiments, at least two of the immunogenic proteins (or immunogenic fragments and/or immunogenic variants thereof) of the plurality are from the same pathogens (e.g., the same virus). In some embodiments, at least two of the immunogenic proteins (or immunogenic fragments and/or immunogenic variants thereof) of the plurality are from different strains of the same pathogen (e.g., different strains of the same virus). In some embodiments, each immunogenic protein of the plurality comprises a different variant of the same immunogenic protein.

In some embodiments, at least one of the immunogenic proteins (or immunogenic fragment and/or immunogenic variant thereof) of the plurality is a first virus respiratory virus immunogenic protein (or immunogenic fragment and/or immunogenic variant thereof); and at least one of the immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the immunogenic proteins (or immunogenic fragments and/or immunogenic variants thereof) of the plurality are variants of the same tumor associated immunogen.

### 5.13 Vaccine Compositions

In some embodiments, one or more of a hIL-10R binding agent (e.g., a hIL-10R binding protein) (or a functional fragment and/or functional variant thereof) (e.g., described herein, see, e.g., § 5.2) (or a fusion protein or conjugate thereof) (or a nucleic acid molecule encoding the hIL-10R binding agent (e.g., the hIL-10R binding protein) (or the functional fragment and/or functional variant thereof) (e.g., described herein, see, e.g., § 5.4)) (or a fusion protein or conjugate thereof); an immunogen (e.g., an immunogenic protein or an immunogenic fragment or variant thereof)) (e.g., described herein, see, e.g., § 5.5) (or a nucleic acid molecule encoding the immunogenic protein or the immunogenic fragment or variant thereof)) (e.g., described herein, see, e.g., § 5.6)); and/or an IGIP (e.g., hIGIP) protein (or a functional fragment and/or functional variant thereof) (e.g., described herein, see, e.g., § 5.7) (or a nucleic acid molecule encoding the IGIP (e.g., hIGIP) protein or the functional fragment and/or functional variant thereof)) (e.g., described herein, see, e.g., § 5.8)) and/or the compositions described herein (see, e.g., §§ 5.12, 5.13, 5.20), form the basis for a vaccine composition (e.g., a vaccine prime composition, a vaccine booster composition).

Provided herein are vaccine compositions (e.g., a vaccine prime composition, a vaccine booster composition) (e.g., vaccine against a pathogen or tumor); wherein the improvement comprises one or more of a hIL-10R binding agent (e.g., a hIL-10R binding protein) (or a functional fragment and/or functional variant thereof) (e.g., described herein, see, e.g., § 5.2) (or a fusion protein or conjugate thereof) (or a nucleic acid molecule encoding the hIL-10R binding agent (e.g., the hIL-10R binding protein) (or the functional fragment and/or functional variant thereof) (e.g., described herein, see, e.g., § 5.4)) (or a fusion protein or conjugate thereof) that e.g., provides an enhanced immune response (e.g., provides a boost).

In some embodiments, the vaccine composition comprises an immunogen (e.g., an immunogenic protein (or an immunogenic fragment or variant thereof) (e.g., described herein, see, e.g., § 5.5) (or a fusion protein or conjugate thereof) (or a nucleic acid molecule encoding the immunogenic protein (or the immunogenic fragment or variant thereof) (e.g., described herein, see, e.g., § 5.6))) (or a fusion protein or conjugate thereof).

In some embodiments, the vaccine composition comprises a hIL-10R binding agent (e.g., a hIL-10R binding protein) (or functional fragment and/or functional variant thereof) (e.g., described herein, see, e.g., § 5.2) (or a fusion protein or conjugate thereof) (or a nucleic acid molecule encoding a hIL-10R binding agent (e.g., the hIL-10R binding protein) (or the functional fragment and/or functional variant thereof) (e.g., described herein, see, e.g., § 5.4)) (or a fusion protein or conjugate thereof).

In some embodiments, the vaccine composition comprises a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)); and an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))).

In some embodiments, the vaccine composition comprises an IGIP (e.g., hIGIP) protein (e.g., or a functional fragment and/or a functional variant thereof) (e.g., described herein, see, e.g., § 5.7) (or a fusion protein or conjugate thereof) (or a nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof).

In some embodiments, the vaccine composition comprises a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)); an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))); and an IGIP (e.g., hIGIP) protein (e.g., or a functional fragment and/or a functional variant thereof) (e.g., described herein, see, e.g., § 5.7) (or a fusion protein or conjugate thereof) (or a nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof).

In some embodiments, the vaccine composition is a vaccine prime or booster composition of a prime-boost vaccine regimen.

In some embodiments, prime-boost vaccine regimens described herein can comprise a first administration of an immunogen to a subject and sometime thereafter administration of an adjuvant alone e.g., (one or more of a hIL-10R binding agent (e.g., a hIL-10R binding protein) (or a functional fragment and/or functional variant thereof) (e.g., described herein, see, e.g., § 5.2) (or a nucleic acid molecule encoding the hIL-10R binding agent (e.g., the hIL-10R binding protein) (or the functional fragment and/or functional variant thereof) (e.g., described herein, see, e.g., § 5.4))).

In some embodiments, prime-boost vaccine regimens described herein can comprise a first administration of an immunogen to a subject and sometime thereafter administration of an adjuvant (e.g., (one or more of a hIL-10R binding agent (e.g., a hIL-10R binding protein) (or a functional fragment and/or functional variant thereof) (e.g., described herein, see, e.g., § 5.2) (or a nucleic acid molecule encoding the hIL-10R binding agent (e.g., the hIL-10R binding protein) (or the functional fragment and/or functional variant thereof) (e.g., described herein, see, e.g., § 5.4)))) in combination with a second dose of a second immunogen to the subject. The first and second immunogens can be the same or different.

The prime and the boost can be the same (homologous prime-boost vaccine regimen) or different vaccine forms (heterologous prime-boost vaccine regimen). For example, the prime can be a nucleic acid-based vaccine while the booster may be a protein-based vaccine. See, e.g., Lu S. Heterologous prime-boost vaccination, Curr Opin Immunol. 2009; 21(3):346-351. doi:10.1016/j.coi.2009.05.016, the entire contents of which is incorporated by reference herein for all purposes.

### 5.13.1 Vaccine Prime Compositions

In some embodiments, the vaccine prime composition comprises an immunogen (e.g., an immunogenic protein (or an immunogenic fragment or variant thereof) (e.g., described herein, see, e.g., § 5.5) (or a fusion protein or conjugate thereof) (or a nucleic acid molecule encoding the immunogenic protein (or the immunogenic fragment or variant thereof) (e.g., described herein, see, e.g., § 5.6))) (or a fusion protein or conjugate thereof).

In some embodiments, the vaccine prime composition comprises an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))); and a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine prime composition comprises an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))); which is administered in combination with a vaccine prime composition comprising a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine prime composition comprises a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine prime composition further comprises an IGIP (e.g., hIGIP) protein (e.g., or a functional fragment and/or a functional variant thereof) (e.g., described herein, see, e.g., § 5.7) (or a fusion protein or conjugate thereof) (or a nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof).

### 5.13.1.1 Protein-Based Vaccine Prime Compositions

In some embodiments, the vaccine prime composition is a protein-based vaccine composition comprising an immunogenic protein (or immunogenic fragment and/or immunogenic variant thereof) (e.g., described herein, see, e.g., § 5.5). In some embodiments, the vaccine prime composition comprises an immunogenic protein described in § 5.5. In some embodiments, the immunogenic proteins, of the composition are formulated in one or more carrier (e.g., a carrier described herein (see, e.g., § 5.15)).

In some embodiments, the vaccine prime composition comprises an IGIP (e.g., hIGIP) protein (e.g., or a functional fragment and/or a functional variant thereof) (e.g., described herein, see, e.g., § 5.7) (or a fusion protein or conjugate thereof). In some embodiments, the vaccine prime composition comprises an IGIP (e.g., hIGIP) protein (e.g., or a functional fragment and/or a functional variant thereof) described in § 5.7. In some embodiments, the IGIP (e.g., hIGIP) protein, of the composition are formulated in one or more carrier (e.g., a carrier described herein (see, e.g., § 5.15)).

In some embodiments, the compositions are pharmaceutical compositions (e.g., described herein, e.g., see § 5.20). In some embodiments, the compositions comprise an adjuvant (e.g., described herein, e.g., see § 5.19).

### (i) Plurality of Immunogens

In some embodiments, the vaccine prime composition comprises a plurality of immunogenic proteins (or immunogenic fragments and/or immunogenic variants thereof) (e.g., described herein, see, e.g., § 5.5)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more immunogenic proteins (or immunogenic fragments and/or immunogenic variants thereof). In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 immunogenic proteins (or immunogenic fragments and/or immunogenic variants thereof). In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 immunogenic proteins (or immunogenic fragments and/or immunogenic variants thereof).

In some embodiments, at least two (e.g., at least 2, 3, 4, 5, 6, 7, 8, 9, 10, or more) of the immunogenic proteins (or immunogenic fragments and/or immunogenic variants thereof) of the plurality are different. In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each immunogenic protein (or immunogenic fragment and/or immunogenic variant thereof) of the plurality is different. In some embodiments, each immunogenic protein (or immunogenic fragment and/or immunogenic variant thereof) of the plurality comprises a different amino acid sequence relative to the amino acid sequence of the other members of the plurality. In some embodiments, the amino acid sequence of each immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the immunogenic proteins (or immunogenic fragments and/or immunogenic variants thereof) of the plurality are from different pathogens (e.g., different viruses). In some embodiments, at least two (e.g., at least 2, 3, 4, 5, 6, 7, 8, 9, 10, or more) of the immunogenic proteins (or immunogenic fragments and/or immunogenic variants thereof) of the plurality are from the same pathogens (e.g., the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality are from different strains of the same pathogen (e.g., different strains of the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different variant of the same immunogenic protein.

In some embodiments, at least two of the immunogenic proteins (or immunogenic fragments and/or immunogenic variants thereof) of the plurality are from different pathogens (e.g., different viruses). In some embodiments, at least two of the immunogenic proteins (or immunogenic fragments and/or immunogenic variants thereof) of the plurality are from the same pathogens (e.g., the same virus). In some embodiments, at least two of the immunogenic proteins (or immunogenic fragments and/or immunogenic variants thereof) of the plurality are from different strains of the same pathogen (e.g., different strains of the same virus). In some embodiments, each immunogenic protein of the plurality comprises a different variant of the same immunogenic protein.

In some embodiments, at least one of the immunogenic proteins (or immunogenic fragment and/or immunogenic variant thereof) of the plurality is a first virus respiratory virus immunogenic protein (or immunogenic fragment and/or immunogenic variant thereof); and at least one of the immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the immunogenic proteins (or immunogenic fragments and/or immunogenic variants thereof) of the plurality are variants of the same tumor associated immunogen.

### 5.13.1.2 Nucleic Acid-Based Vaccine Prime Compositions

In some embodiments, the prime portion of the regimen comprises is a nucleic acid-based vaccine prime composition comprising a nucleic acid molecule encoding an immunogenic protein (or an immunogenic fragment or variant thereof) (e.g., described herein, see, e.g., § 5.6)). In some embodiments, the prime portion of the regimen comprises a nucleic acid molecule encoding an immunogenic protein described in § 5.6.

In some embodiments, the prime portion of the regimen comprises a nucleic acid molecule comprising a coding region encoding an IGIP (e.g., hIGIP) protein (or a functional fragment and/or functional variant thereof) (e.g., described herein, see, e.g., § 5.7). In some embodiments, the prime portion of the regimen comprises a nucleic acid molecule comprising a coding region encoding an IGIP (e.g., hIGIP) protein (or a functional fragment and/or functional variant thereof) described in § 5.8.

In some embodiments, the nucleic acid molecules are comprised within one or more vectors (e.g., vectors described herein (see, e.g., § 5.14)). In some embodiments, the nucleic acid molecules or the vectors of the composition are formulated in one or more carrier (e.g., a carrier described herein (see, e.g., § 5.15)). In some embodiments, the compositions are pharmaceutical compositions (e.g., described herein, e.g., see § 5.20). In some embodiments, the compositions comprise an adjuvant (e.g., described herein, e.g., see § 5.19). Nucleic acid molecules can be generated using common methods known in the art and described above in § 5.6.

### (i) Plurality of Immunogens

In some embodiments, the vaccine prime composition comprises a plurality of nucleic acid molecules each comprising a coding region encoding an immunogenic protein (or immunogenic fragment and/or immunogenic variant thereof) (e.g., described herein, see, e.g., § 5.6)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more nucleic acid molecules. In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleic acid molecules. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 nucleic acid molecules.

In some embodiments, each of the nucleic acid molecules of the plurality are part of the same larger nucleic acid molecule. In some embodiments, each of the nucleic acid molecules of the plurality are separate (i.e., not connected) nucleic acid molecules. In some embodiments, at least two of the nucleic acid molecules of the plurality are part of the same larger nucleic acid molecule. In some embodiments, at least two of the nucleic acid molecules of the plurality are separate (i.e., not connected) nucleic acid molecules. In some embodiments, at least two of the nucleic acid molecules of the plurality are part of the same larger nucleic acid molecule; and at least one (e.g., at least 2, 3, 4, 5, etc.) of the nucleic acid molecules of the plurality is a separate (i.e., not connected) nucleic acid molecule.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the encoded immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each encoded immunogenic protein (or immunogenic fragment and/or immunogenic variant thereof) of the plurality is different. In some embodiments, each encoded immunogenic protein (or immunogenic fragment and/or immunogenic variant thereof) of the plurality comprises a different amino acid sequence relative to the amino acid sequence of the other members of the plurality. In some embodiments, the amino acid sequence of each encoded immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different organisms (e.g., different viruses). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from the same organisms (e.g., the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different strains of the same organism (e.g., different strains of the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different variant of the same immunogenic protein.

In some embodiments, at least two of the encoded immunogenic proteins (or immunogenic fragments and/or immunogenic variants thereof) of the plurality are from different organisms (e.g., different viruses). In some embodiments, at least two of the encoded immunogenic proteins (or immunogenic fragments and/or immunogenic variants thereof) of the plurality are from the same organisms (e.g., the same virus). In some embodiments, at least two of the encoded immunogenic proteins (or immunogenic fragments and/or immunogenic variants thereof) of the plurality are from different strains of the same organism (e.g., different strains of the same virus). In some embodiments, each encoded immunogenic protein of the plurality comprises a different variant of the same immunogenic protein.

In some embodiments, at least one of the encoded immunogenic proteins (or immunogenic fragment and/or immunogenic variant thereof) of the plurality is a first virus respiratory virus immunogenic protein (or immunogenic fragment and/or immunogenic variant thereof); and at least one of the encoded immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are variants of the same tumor associated immunogen.

### 5.13.1.3 Vaccine Prime Formulation for Administration

The vaccine prime composition (e.g., protein-based vaccine composition, nucleic acid-based vaccine prime composition) can be formulated for any route of administration to a subject (e.g., as described herein, see, e.g., § 5.20). In some embodiments, the vaccine prime composition is formulated for parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like. In some embodiments, the vaccine prime composition is formulated for intramuscular, subcutaneous, or intranasal administration. In some embodiments, the vaccine prime composition is formulated for intramuscular or subcutaneous administration. In some embodiments, the vaccine prime composition is formulated for intranasal administration.

### 5.13.2 Vaccine Booster Compositions

In some embodiments, the vaccine booster composition comprises an immunogen (e.g., an immunogenic protein (or an immunogenic fragment or variant thereof) (e.g., described herein, see, e.g., § 5.5) (or a fusion protein or conjugate thereof) (or a nucleic acid molecule encoding the immunogenic protein (or the immunogenic fragment or variant thereof) (e.g., described herein, see, e.g., § 5.6))) (or a fusion protein or conjugate thereof).

In some embodiments, the vaccine booster composition comprises an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))); and a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine booster composition comprises an immunogen (e.g., an immunogenic protein (or immunogenic fragment and/or immunogenic variant thereof) (e.g., described herein, see, e.g., § 5.5) (or a nucleic acid molecule encoding the immunogenic protein (or immunogenic fragment and/or immunogenic variant thereof) (e.g., described herein, see, e.g., § 5.6))); which is administered in combination with a vaccine booster composition comprising a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine booster composition comprises a hIL-10R binding agent (e.g., a hIL-10R binding protein) (or functional fragment and/or functional variant thereof) (e.g., described herein, see, e.g., § 5.2) (or a nucleic acid molecule encoding a hIL-10R binding agent (e.g., the hIL-10R binding protein) (or the functional fragment and/or functional variant thereof) (e.g., described herein, see, e.g., § 5.4)).

In some embodiments, the vaccine booster composition further comprises an IGIP (e.g., hIGIP) protein (e.g., or a functional fragment and/or a functional variant thereof) (e.g., described herein, see, e.g., § 5.7) (or a fusion protein or conjugate thereof) (or a nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof).

### 5.13.2.1 Protein-Based Vaccine Booster Compositions

In some embodiments, the vaccine booster composition is a protein-based vaccine composition comprising a hIL-10R binding protein (or functional fragment and/or functional variant thereof) (e.g., described herein, see, e.g., § 5.2). In some embodiments, the vaccine booster composition comprises a hIL-10R binding protein (or functional fragment and/or functional variant thereof) described in § 5.2. In some embodiments, the IL-10R binding proteins (or functional fragments or functional variants thereof), of the composition are formulated in one or more carrier (e.g., a carrier described herein (see, e.g., § 5.15)).

In some embodiments, the vaccine booster composition is a protein-based vaccine composition comprising a hIL-10R binding protein (or functional fragment and/or functional variant thereof) (e.g., described herein, see, e.g., § 5.2) and an immunogenic protein (or immunogenic fragment and/or immunogenic variant thereof) (e.g., described herein, see, e.g., § 5.5). In some embodiments, the vaccine booster composition comprises a hIL-10R binding protein (or functional fragment and/or functional variant thereof) described in § 5.2 and an immunogenic protein (or immunogenic fragment and/or immunogenic variant thereof) described herein in § 5.5.

In some embodiments, the vaccine booster composition comprises an IGIP (e.g., hIGIP) protein (e.g., or a functional fragment and/or a functional variant thereof) (e.g., described herein, see, e.g., § 5.7) (or a fusion protein or conjugate thereof). In some embodiments, the vaccine booster composition comprises an IGIP (e.g., hIGIP) protein (e.g., or a functional fragment and/or a functional variant thereof) described in § 5.7. In some embodiments, the IGIP (e.g., hIGIP) protein, of the composition are formulated in one or more carrier (e.g., a carrier described herein (see, e.g., § 5.15)).

In some embodiments, the compositions are pharmaceutical compositions (e.g., described herein, e.g., see § 5.20). In some embodiments, the compositions comprise an adjuvant (e.g., described herein, e.g., see § 5.19).

### (i) Plurality of Immunogens

In some embodiments, the vaccine booster composition comprises a plurality of immunogenic proteins (or immunogenic fragments and/or immunogenic variants thereof) (e.g., described herein, see, e.g., § 5.5)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more immunogenic proteins (or immunogenic fragments and/or immunogenic variants thereof). In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 immunogenic proteins (or immunogenic fragments and/or immunogenic variants thereof). In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 immunogenic proteins (or immunogenic fragments and/or immunogenic variants thereof).

In some embodiments, at least two (e.g., at least 2, 3, 4, 5, 6, 7, 8, 9, 10, or more) of the immunogenic proteins (or immunogenic fragments and/or immunogenic variants thereof) of the plurality are different. In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each immunogenic protein (or immunogenic fragment and/or immunogenic variant thereof) of the plurality is different. In some embodiments, each immunogenic protein (or immunogenic fragment and/or immunogenic variant thereof) of the plurality comprises a different amino acid sequence relative to the amino acid sequence of the other members of the plurality. In some embodiments, the amino acid sequence of each immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the immunogenic proteins (or immunogenic fragments and/or immunogenic variants thereof) of the plurality are from different pathogens (e.g., different viruses). In some embodiments, at least two (e.g., at least 2, 3, 4, 5, 6, 7, 8, 9, 10, or more) of the immunogenic proteins (or immunogenic fragments and/or immunogenic variants thereof) of the plurality are from the same pathogens (e.g., the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality are from different strains of the same pathogen (e.g., different strains of the same virus). In some embodiments, at least two (e.g., at least 2, 3, 4, 5, 6, 7, 8, 9, 10, or more) of the immunogenic proteins (or immunogenic fragments and/or immunogenic variants thereof) of the plurality comprise a different variant of the same immunogenic protein.

In some embodiments, at least two of the immunogenic proteins (or immunogenic fragments and/or immunogenic variants thereof) of the plurality are from different pathogens (e.g., different viruses). In some embodiments, at least two of the immunogenic proteins (or immunogenic fragments and/or immunogenic variants thereof) of the plurality are from the same pathogens (e.g., the same virus). In some embodiments, at least two of the immunogenic proteins (or immunogenic fragments and/or immunogenic variants thereof) of the plurality are from different strains of the same pathogen (e.g., different strains of the same virus). In some embodiments, each immunogenic protein of the plurality comprises a different variant of the same immunogenic protein.

In some embodiments, at least one of the immunogenic proteins (or immunogenic fragment and/or immunogenic variant thereof) of the plurality is a first virus respiratory virus immunogenic protein (or immunogenic fragment and/or immunogenic variant thereof); and at least one of the immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the immunogenic proteins (or immunogenic fragments and/or immunogenic variants thereof) of the plurality are variants of the same tumor associated immunogen.

In some embodiments, the immunogenic proteins (or immunogenic fragments and/or immunogenic variants thereof), of the composition are formulated in one or more carrier (e.g., a carrier described herein (see, e.g., § 5.15)). In some embodiments, the compositions are pharmaceutical compositions (e.g., described herein, e.g., see § 5.20). In some embodiments, the compositions comprise an adjuvant (e.g., described herein, e.g., see § 5.19).

### 5.13.2.2 Nucleic Acid-Based Vaccine Booster Compositions

In some embodiments, the vaccine booster composition is a nucleic acid-based vaccine booster composition comprising a nucleic acid molecule encoding a hIL-10R binding protein (or the functional fragment and/or functional variant thereof) (e.g., described herein, see, e.g., § 5.4)). In some embodiments, the vaccine booster composition comprises a nucleic acid molecule encoding a hIL-10R binding protein (or the functional fragment and/or functional variant thereof) described herein in § 5.4. In some embodiments, the nucleic acid molecules are comprised within one or more vectors (e.g., vectors described herein (see, e.g., § 5.14)). In some embodiments, the nucleic acid molecules or the vectors of the composition are formulated in one or more carrier (e.g., a carrier described herein (see, e.g., § 5.15)). In some embodiments, the compositions are pharmaceutical compositions (e.g., described herein, e.g., see § 5.20). In some embodiments, the compositions comprise an adjuvant (e.g., described herein, e.g., see § 5.19). Nucleic acid molecules can be generated using common methods known in the art and described above in § 5.6.

In some embodiments, the vaccine booster composition is a nucleic acid-based vaccine booster composition comprising a nucleic acid molecule encoding a hIL-10R binding protein (or the functional fragment and/or functional variant thereof) (e.g., described herein, see, e.g., § 5.4)) and a nucleic acid molecule encoding the immunogenic protein (or immunogenic fragment and/or immunogenic variant thereof) (e.g., described herein, see, e.g., § 5.6)). In some embodiments, the vaccine booster composition comprises a nucleic acid molecule encoding a hIL-10R binding protein (or the functional fragment and/or functional variant thereof) described in § 5.4 and a nucleic acid molecule encoding the immunogenic protein (or immunogenic fragment and/or immunogenic variant thereof) described in § 5.6.

In some embodiments, the vaccine booster composition comprises a nucleic acid molecule comprising a coding region encoding an IGIP (e.g., hIGIP) protein (or a functional fragment and/or functional variant thereof) (e.g., described herein, see, e.g., § 5.7). In some embodiments, the prime portion of the regimen comprises a nucleic acid molecule comprising a coding region encoding an IGIP (e.g., hIGIP) protein (or a functional fragment and/or functional variant thereof) described in § 5.8.

In some embodiments, the nucleic acid molecules are comprised within one or more vectors (e.g., vectors described herein (see, e.g., § 5.14)). In some embodiments, the nucleic acid molecules or the vectors of the composition are formulated in one or more carrier (e.g., a carrier described herein (see, e.g., § 5.15)). In some embodiments, the compositions are pharmaceutical compositions (e.g., described herein, e.g., see § 5.20). In some embodiments, the compositions comprise an adjuvant (e.g., described herein, e.g., see § 5.19). Nucleic acid molecules can be generated using common methods known in the art and described above in § 5.6.

### (i) Plurality of Immunogens

In some embodiments, the vaccine booster composition comprises a plurality of nucleic acid molecules each comprising a coding region encoding an immunogenic protein (or immunogenic fragment and/or immunogenic variant thereof) (e.g., described herein, see, e.g., § 5.6)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more nucleic acid molecules. In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleic acid molecules. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 nucleic acid molecules.

In some embodiments, each of the nucleic acid molecules of the plurality are part of the same larger nucleic acid molecule. In some embodiments, each of the nucleic acid molecules of the plurality are separate (i.e., not connected) nucleic acid molecules. In some embodiments, at least two of the nucleic acid molecules of the plurality are part of the same larger nucleic acid molecule. In some embodiments, at least two of the nucleic acid molecules of the plurality are separate (i.e., not connected) nucleic acid molecules. In some embodiments, at least two of the nucleic acid molecules of the plurality are part of the same larger nucleic acid molecule; and at least one (e.g., at least 2, 3, 4, 5, etc.) of the nucleic acid molecules of the plurality is a separate (i.e., not connected) nucleic acid molecule.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different amino acid sequence relative to each other. In some embodiments, the amino acid sequence of at least two (e.g., at least 2, 3, 4, 5, 6, 7, 8, 9, 10, or more) of the encoded immunogenic proteins (or immunogenic fragments and/or immunogenic variants thereof) of the plurality comprise at least 1, 2, 3, 4, 5, 6, 7, 8, 9, or 10, or more amino acid variations relative to the amino acid sequence of each other.

In some embodiments, each encoded immunogenic protein (or immunogenic fragment and/or immunogenic variant thereof) of the plurality is different. In some embodiments, each encoded immunogenic protein (or immunogenic fragment and/or immunogenic variant thereof) of the plurality comprises a different amino acid sequence relative to the amino acid sequence of the other members of the plurality. In some embodiments, the amino acid sequence of each encoded immunogenic protein (or immunogenic fragment and/or immunogenic variant thereof) of the plurality comprises at least 1, 2, 3, 4, 5, 6, 7, 8, 9, or 10, or more amino acid variations relative to the amino acid sequence of the other members of the plurality.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different organisms (e.g., different viruses). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from the same organisms (e.g., the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are from different strains of the same organism (e.g., different strains of the same virus). In some embodiments, at least two (e.g., at least 2, 3, 4, 5, 6, 7, 8, 9, 10, or more) of the encoded immunogenic proteins (or immunogenic fragments and/or immunogenic variants thereof) of the plurality comprise a different variant of the same immunogenic protein.

In some embodiments, at least two of the encoded immunogenic proteins (or immunogenic fragments and/or immunogenic variants thereof) of the plurality are from different organisms (e.g., different viruses). In some embodiments, at least two of the encoded immunogenic proteins (or immunogenic fragments and/or immunogenic variants thereof) of the plurality are from the same organisms (e.g., the same virus). In some embodiments, at least two of the encoded immunogenic proteins (or immunogenic fragments and/or immunogenic variants thereof) of the plurality are from different strains of the same organism (e.g., different strains of the same virus). In some embodiments, each encoded immunogenic protein of the plurality comprises a different variant of the same immunogenic protein.

In some embodiments, at least one of the encoded immunogenic proteins (or immunogenic fragment and/or immunogenic variant thereof) of the plurality is a first virus respiratory virus immunogenic protein (or immunogenic fragment and/or immunogenic variant thereof); and at least one of the encoded immunogenic proteins (or immunogenic fragment and/or immunogenic variant thereof) of the plurality is a second respiratory virus immunogenic protein (or immunogenic fragment and/or immunogenic variant thereof). In some embodiments, the first and second respiratory virus immunogenic proteins (or immunogenic fragments and/or immunogenic variants thereof) are any one or more of: derived from the same respiratory virus, derived from different respiratory viruses, derived from different strains of the same respiratory virus; and/or variants of the same immunogenic protein. Exemplary respiratory viruses include, but are not limited to coronaviruses (e.g., a SARS-CoV-2 virus, a SARS-CoV virus, a MERS-CoV SARS-CoV-2 virus), influenza viruses (e.g., influenza A, influenza B), respiratory syncytial viruses (RSV), rhinoviruses, parvoviruses (e.g., B19), parainfluenza viruses, and adenoviruses.

In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are different tumor associated immunogens derived from the same tumor. In some embodiments, at least two (e.g., at least 2, 3, 4, 5, 6, 7, 8, 9, 10, or more) of the encoded immunogenic proteins (or immunogenic fragments and/or immunogenic variants thereof) of the plurality are variants of the same tumor associated immunogen.

### 5.13.2.3 Vaccine Booster Formulation for Administration

The vaccine booster composition (e.g., protein-based vaccine composition, nucleic acid-based vaccine booster composition) can be formulated for any route of administration to a subject (e.g., as described herein, see, e.g., § 5.20). In some embodiments, the vaccine booster composition is formulated for parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like. In some embodiments, the vaccine booster composition is formulated for intramuscular, subcutaneous, or intranasal administration. In some embodiments, the vaccine booster composition is formulated for intramuscular or subcutaneous administration. In some embodiments, the vaccine booster composition is formulated for intranasal administration.

### 5.13.3 Combinations Therapies

Further provided herein are combination therapies (e.g., for use in vaccine regimens (e.g., prime-boost vaccine regimens)). As such, in one aspect, provided herein are combination therapies comprising (a) an immunogen (e.g., described herein) (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof) and (b) a hIL-10R binding agent (e.g., described herein) (e.g., a hIL-10R binding protein (or a functional fragment and/or functional variant thereof) or a nucleic acid molecule comprising a coding region encoding a hIL-10R binding protein (or a functional fragment and/or functional variant thereof).

Provided herein are, e.g., combination therapies for use in vaccine regimens (e.g., prime-boost vaccine regimens), wherein the improvement is a hIL-10R binding agent (e.g., described herein) (e.g., a hIL-10R binding protein (or a functional fragment and/or functional variant thereof) or a nucleic acid molecule comprising a coding region encoding a hIL-10R binding protein (or a functional fragment and/or functional variant thereof) that e.g., provides an enhanced immune response (e.g., provides a boost).

In some embodiments, the combination therapy is utilized in a vaccine regimen. In some embodiments, the combination therapy is utilized in a prime-boost vaccine regimen. In some embodiments, (a) is utilized as a prime vaccine. In some embodiments, (a) is utilized as a booster vaccine. In some embodiments, (b) is utilized as a prime vaccine. In some embodiments, (b) is utilized as a booster vaccine. In some embodiments, (a) is utilized as a prime vaccine and (a) is utilized as a booster vaccine. In some embodiments, (a) is utilized as a prime vaccine and (b) is utilized as a prime vaccine. In some embodiments, (a) is utilized as a booster vaccine and (b) is utilized as a booster vaccine. In some embodiments, (a) is utilized as a prime vaccine and (b) is utilized as a booster vaccine. In some embodiments, (b) is utilized as a prime vaccine and (a) is utilized as a booster vaccine.

In some embodiments, (a) and (b) are not co-formulated. In some embodiments, (a) and (b) are co-formulated. In some embodiments, (a) and (b) are administered simultaneously or sequentially. In some embodiments, (a) and (b) are administered sequentially, wherein (b) is administered after (a). In some embodiments, (a) and (b) are administered simultaneously and are not co-formulated.

### 5.13.3.1 Exemplary Combinations of Vaccine Prime and Booster Compositions

The vaccine prime and vaccine booster compositions of any given vaccine prime-boost regimen (e.g., utilized in any of the methods described herein, see, e.g., § 5.21) can individually comprise any vaccine prime and vaccine booster (e.g., described herein). For example, a vaccine prime can be a nucleic acid-based (e.g., RNA, e.g., mRNA) vaccine and the vaccine booster can be a protein-based vaccine and vice versa. In some embodiments, the vaccine prime comprises a nucleic acid-based (e.g., RNA, e.g., mRNA) vaccine and the vaccine booster comprises a protein-based vaccine. In some embodiments, the vaccine prime comprises a protein-based vaccine and the vaccine booster comprises a nucleic acid-based (e.g., RNA, e.g., mRNA) vaccine. In some embodiments, the vaccine prime comprises a protein-based vaccine and the vaccine booster comprises a protein-based vaccine. In some embodiments, the vaccine prime comprises a nucleic acid-based (e.g., RNA, e.g., mRNA) vaccine and the vaccine booster comprises a nucleic acid-based (e.g., RNA, e.g., mRNA) vaccine.

In some embodiments the prime boost regime comprises a prime vaccine comprising (a) an immunogen (e.g., described herein) (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof); and a boost vaccine composition of comprising (b) a hIL-10R binding agent (e.g., described herein) (e.g., a hIL-10R binding protein (or a functional fragment and/or functional variant thereof) or a nucleic acid molecule comprising a coding region encoding a hIL-10R binding protein (or a functional fragment and/or functional variant thereof).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a hIL-10R binding protein (or the functional fragment and/or functional variant thereof) (e.g., described herein).

In some embodiments, the vaccine prime composition comprises an immunogenic protein (or immunogenic fragment and/or immunogenic variant thereof) (e.g., described herein) and the vaccine booster comprises a hIL-10R binding protein (or the functional fragment and/or functional variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a nucleic acid molecule (e.g., RNA, e.g., mRNA) encoding a hIL-10R binding protein (or the functional fragment and/or functional variant thereof) (e.g., described herein).

In some embodiments, the vaccine prime composition comprises an immunogenic protein (or immunogenic fragment and/or immunogenic variant thereof) (e.g., described herein) and the vaccine booster comprises a hIL-10R binding protein (or the functional fragment and/or functional variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first nucleic acid molecule (e.g., RNA, e.g., mRNA) encoding a hIL-10R binding protein (or the functional fragment and/or functional variant thereof) (e.g., described herein) and a second nucleic acid molecule (e.g., RNA, e.g., mRNA) encoding an immunogenic protein (or immunogenic fragment and/or immunogenic variant thereof) (e.g., described herein). The first and second nucleic acid molecules can be disposed on the same larger nucleic acid molecule (i.e., operably connected) or on separate nucleic acid molecules.

In some embodiments, the vaccine prime composition comprises an immunogenic protein (or immunogenic fragment and/or immunogenic variant thereof) (e.g., described herein) and the vaccine booster comprises a first nucleic acid molecule (e.g., RNA, e.g., mRNA) encoding a hIL-10R binding protein (or the functional fragment and/or functional variant thereof) (e.g., described herein) and a second nucleic acid molecule (e.g., RNA, e.g., mRNA) encoding an immunogenic protein (or immunogenic fragment and/or immunogenic variant thereof) (e.g., described herein). The first and second nucleic acid molecules can be disposed on the same larger nucleic acid molecule (i.e., operably connected) or on separate nucleic acid molecules.

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a hIL-10R binding protein (or the functional fragment and/or functional variant thereof) (e.g., described herein) and an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a hIL-10R binding protein (or the functional fragment and/or functional variant thereof) (e.g., described herein) and an immunogenic protein (or immunogenic fragment and/or immunogenic variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first composition comprising hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first composition comprising a nucleic acid molecule (e.g., RNA, e.g., mRNA) encoding a hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first composition comprising a nucleic acid molecule (e.g., RNA, e.g., mRNA) encoding a hIL-10R binding protein (or the functional fragment and/or functional variant thereof) (e.g., described herein) and a second composition comprising an immunogenic protein (or immunogenic fragment and/or immunogenic variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first composition comprising a hIL-10R binding protein (or the functional fragment and/or functional variant thereof) (e.g., described herein) and a second composition comprising an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a nucleic acid molecule (e.g., RNA, e.g., mRNA) encoding a hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a hIL-10R binding protein (or the functional fragment and/or functional variant thereof) (e.g., described herein) and a second composition comprising an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a nucleic acid molecule (e.g., RNA, e.g., mRNA) encoding a hIL-10R binding protein (or the functional fragment and/or functional variant thereof) (e.g., described herein) and a second composition comprising an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises an immunogenic protein (or immunogenic fragment and/or immunogenic variant thereof) (e.g., described herein) and the vaccine booster comprises a first composition comprising a hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

In some embodiments, the vaccine prime composition comprises a nucleic acid molecule (e.g., RNA, e.g., mRNA) encoding an immunogenic protein (or immunogenic fragment and/or immunogenic variant thereof) (e.g., described herein) and the vaccine booster comprises a first composition comprising hIL-10R binding protein (or the functional fragment and/or functional variant thereof) (e.g., described herein) and a second composition comprising a nucleic acid molecule (e.g., RNA, e.g., mRNA) encoding an immunogenic protein (or immunogenic fragment and/or immunogenic variant thereof) (e.g., described herein).

Likewise, the route of administration of the vaccine prime and the vaccine booster of any given prime-boost vaccine regimen can be the same or different. For example, the vaccine prime can be administered intramuscularly or subcutaneously and the vaccine boost administered intranasally. In some embodiments, the vaccine prime composition is administered intramuscularly or subcutaneously and the vaccine boost administered intranasally. In some embodiments, the vaccine prime composition is administered intramuscularly or subcutaneously and the vaccine boost administered intramuscularly or subcutaneously. In some embodiments, the vaccine prime composition is administered intranasally and the vaccine boost administered intranasally.

In some embodiments, the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein); and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein). Likewise, these two booster compositions do not have to be administered by the same route of administration.

In some embodiments, the first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) is administered intranasally; and the second composition comprising the immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally.

In some embodiments, the first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) is administered intranasally; and the second composition comprising the immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously.

In some embodiments, the first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) is administered intramuscularly or subcutaneously; and the second composition comprising the immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously.

In some embodiments, the first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) is administered intramuscularly or subcutaneously; and the second composition comprising the immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intranasally; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intranasally.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intranasally; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intranasally.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intramuscularly or subcutaneously; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intranasally.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intranasally; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intramuscularly or subcutaneously.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intramuscularly or subcutaneously; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intramuscularly or subcutaneously.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intramuscularly or subcutaneously; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intranasally.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intramuscularly or subcutaneously; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intranasally; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intramuscularly or subcutaneously.

In some embodiments, the vaccine prime composition comprising immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) is administered intranasally; and the booster comprises two compositions a first composition comprising a hIL-10R binding proteins (or functional fragment and/or functional variant thereof) (e.g., described herein) (or a nucleic acid molecule (e.g., RNA, e.g., mRNA) encoding a hIL-10R binding protein (or the functional fragment and/or functional variant thereof) (e.g., described herein) administered intramuscularly or subcutaneously; and a second composition comprising an immunogenic protein (or immunogenic fragment and/or immunogenic variant thereof) (e.g., described herein) (or a nucleic acid molecule (e.g., RNA, e.g., mRNA) encoding the immunogenic protein (or immunogenic fragment and/or immunogenic variant thereof) (e.g., described herein) administered intramuscularly or subcutaneously.

### 5.14 Vectors

In some embodiments, the nucleic acid molecules described herein (DNA or RNA (e.g., mRNA)) (see, e.g., §§ 5.4, 5.6, 5.11, 5.12.1, 5.13.1.2, 5.13.2.2) are contained in a vector (e.g., a non-viral vector (e.g., a plasmid), a viral vector). Thus, in one aspect, also provided herein are vectors (e.g., non-viral vectors (e.g., plasmids) viral vectors) comprising one or more nucleic acid molecule described herein (e.g., nucleic acid molecules encoding a hIL-10R binding protein (or functional fragment and/or functional variant thereof) (e.g., described herein, see, e.g., § 5.2); nucleic acid molecules encoding an immunogenic protein (or immunogenic fragment and/or immunogenic variant thereof) (e.g., described herein, see, e.g., § 5.5); nucleic acid molecules encoding an IGIP protein (or functional fragment and/or functional variant thereof) (e.g., described herein, see, e.g., § 5.7); polycistronic nucleic acid molecules (e.g., described herein, see, e.g., § 5.11), etc.). Such vectors can be easily manipulated by methods well known to the ordinary person of skill in the art. The vector used can be any vector that is suitable for cloning nucleic acid molecules that can be used for transcription of the nucleic acid molecule of interest.

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an immunogenic protein described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™ pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

In some embodiments, the vector is a plasmid. A person of ordinary skill in the art is aware of suitable plasmids for expression of the DNA of interest. For example, Suitable plasmid DNA may be generated to allow efficient production of the encoded immunogens in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 5.15 Carriers

In some embodiments, one or more agent described herein, e.g., an hIL-10R binding agent described herein (e.g., a hIL-10R binding protein described herein) (or a functional fragment and/or functional variant thereof) (e.g., described herein, see, e.g., § 5.2) (or a fusion protein or conjugate thereof) (or a nucleic acid molecule encoding the hIL-10R binding protein) (or the functional fragment and/or functional variant thereof) (e.g., described herein, see, e.g., § 5.4)); an immunogen described herein (e.g., an immunogenic protein (or an immunogenic fragment and/or immunogenic variant thereof) (e.g., described herein, see, e.g., § 5.5) (or the nucleic acid molecule encoding the immunogenic protein (or the immunogenic fragment and/or immunogenic variant thereof) (e.g., described herein, see, e.g., § 5.6))); an IGIP (e.g., hIGIP) protein described herein (e.g., or the functional fragment or the functional variant thereof) (e.g., described herein, see, e.g., § 5.7) (or a fusion protein or conjugate thereof) (or the nucleic acid molecule encoding the IGIP (e.g., hIGIP) protein (e.g., or the functional fragment or the functional variant thereof)) (e.g., described herein, see, e.g., § 5.8))); a polycistronic nucleic acid molecule described herein (see, e.g., § 5.11), a combination composition described herein (see, e.g., § 5.12); a vaccine composition described herein (see, e.g., § 5.13); a vector described herein (see, e.g., § 5.14)); a pharmaceutical composition (see, e.g., § 5.20); and/or a host cell described herein (see, e.g., § 5.18)), is formulated within one or more carrier.

Therefore, in one aspect, provided herein are carriers comprising any one or more agent described herein (e.g., an hIL-10R binding agent described herein (e.g., a hIL-10R binding protein described herein) (or a functional fragment and/or functional variant thereof) (e.g., described herein, see, e.g., § 5.2) (or a fusion protein or conjugate thereof) (or a nucleic acid molecule encoding the hIL-10R binding protein) (or the functional fragment and/or functional variant thereof) (e.g., described herein, see, e.g., § 5.4)); an immunogen described herein (e.g., an immunogenic protein (or an immunogenic fragment and/or immunogenic variant thereof) (e.g., described herein, see, e.g., § 5.5) (or the nucleic acid molecule encoding the immunogenic protein (or the immunogenic fragment and/or immunogenic variant thereof) (e.g., described herein, see, e.g., § 5.6))); an IGIP (e.g., hIGIP) protein described herein (e.g., or the functional fragment or the functional variant thereof) (e.g., described herein, see, e.g., § 5.7) (or a fusion protein or conjugate thereof) (or the nucleic acid molecule encoding the IGIP (e.g., hIGIP) protein (e.g., or the functional fragment or the functional variant thereof)) (e.g., described herein, see, e.g., § 5.8))); a polycistronic nucleic acid molecule described herein (see, e.g., § 5.11), a combination composition described herein (see, e.g., § 5.12); a vaccine composition described herein (see, e.g., § 5.13); a vector described herein (see, e.g., § 5.14)); a pharmaceutical composition (see, e.g., § 5.20); and/or a host cell described herein (see, e.g., § 5.18))).

In one aspect, provided herein are methods of making carriers (e.g., lipid nanoparticles, e.g., described herein) comprising combining one or more lipid (e.g., described herein; e.g., any one of more a cationic lipid (e.g., an ionizable lipid), a non-cationic lipid (e.g., phospholipid), a structural lipid (e.g., cholesterol), and/or a PEG-modified lipid) and any one or more agent described herein (e.g., an hIL-10R binding agent described herein (e.g., a hIL-10R binding protein described herein) (or a functional fragment and/or functional variant thereof) (e.g., described herein, see, e.g., § 5.2) (or a fusion protein or conjugate thereof) (or a nucleic acid molecule encoding the hIL-10R binding protein) (or the functional fragment and/or functional variant thereof) (e.g., described herein, see, e.g., § 5.4)); an immunogen described herein (e.g., an immunogenic protein (or an immunogenic fragment and/or immunogenic variant thereof) (e.g., described herein, see, e.g., § 5.5) (or the nucleic acid molecule encoding the immunogenic protein (or the immunogenic fragment and/or immunogenic variant thereof) (e.g., described herein, see, e.g., § 5.6))); an IGIP (e.g., hIGIP) protein described herein (e.g., or the functional fragment or the functional variant thereof) (e.g., described herein, see, e.g., § 5.7) (or a fusion protein or conjugate thereof) (or the nucleic acid molecule encoding the IGIP (e.g., hIGIP) protein (e.g., or the functional fragment or the functional variant thereof)) (e.g., described herein, see, e.g., § 5.8))); a polycistronic nucleic acid molecule described herein (see, e.g., § 5.11), a combination composition described herein (see, e.g., § 5.12); a vaccine composition described herein (see, e.g., § 5.13); a vector described herein (see, e.g., § 5.14)); a pharmaceutical composition (see, e.g., § 5.20); and/or a host cell described herein (see, e.g., § 5.18))); wherein the one or more agent is encapsulated or associated within the carrier (e.g., LNP).

Any of the agents described herein can be encapsulated within a carrier, chemically conjugated to a carrier, associated with the carrier. In this context, the term “associated” refers to the essentially stable combination of an agent described herein with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of an agent described herein into a carrier (e.g., a lipid-based carrier, e.g., an LNP, liposome, lipoplex, and/or nanoliposome) wherein the agent is entirely contained within the interior space of the carrier (e.g., the lipid-based carrier, e.g., the LNP, liposome, lipoplex, and/or nanoliposome).

Exemplary carriers includes, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.15.1.

### 5.15.1 Lipid Based Carriers/Lipid Nanoformulations

In some embodiments, an agent described herein is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, an agent described herein is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein is associated with one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein is encapsulated in LNPs (e.g., as described herein).

The agent may be completely or partially located in the interior space of the LNPs, liposomes, lipoplexes, and/or nanoliposomes, within the lipid layer/membrane, or associated with the exterior surface of the lipid layer/membrane. One purpose of incorporating an agent described herein into LNPs, liposomes, lipoplexes, and/or nanoliposomes is to protect the agent from an environment which may contain enzymes or chemicals or conditions that degrade the agent from molecules or conditions that cause the rapid excretion of the agent. Moreover, incorporating an agent described herein into LNPs, liposomes, lipoplexes, and/or nanoliposomes may promote the uptake of the agent, and hence, may enhance the therapeutic effect of the agent. Accordingly, incorporating a an agent described herein, into LNPs, liposomes, lipoplexes, and/or nanoliposomes may be particularly suitable for a pharmaceutical composition described herein, e.g., for intramuscular and/or intradermal administration.

In some embodiments, an agent described herein is formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more an agent described herein.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more an agent described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 5.15.1.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N-(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

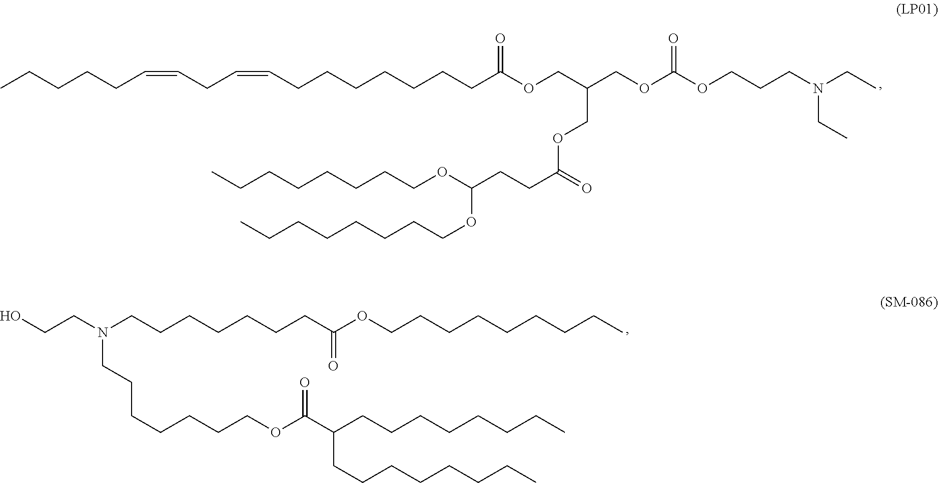 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCOC(=O)C(CCCCCCCC)CCCCCCCC]

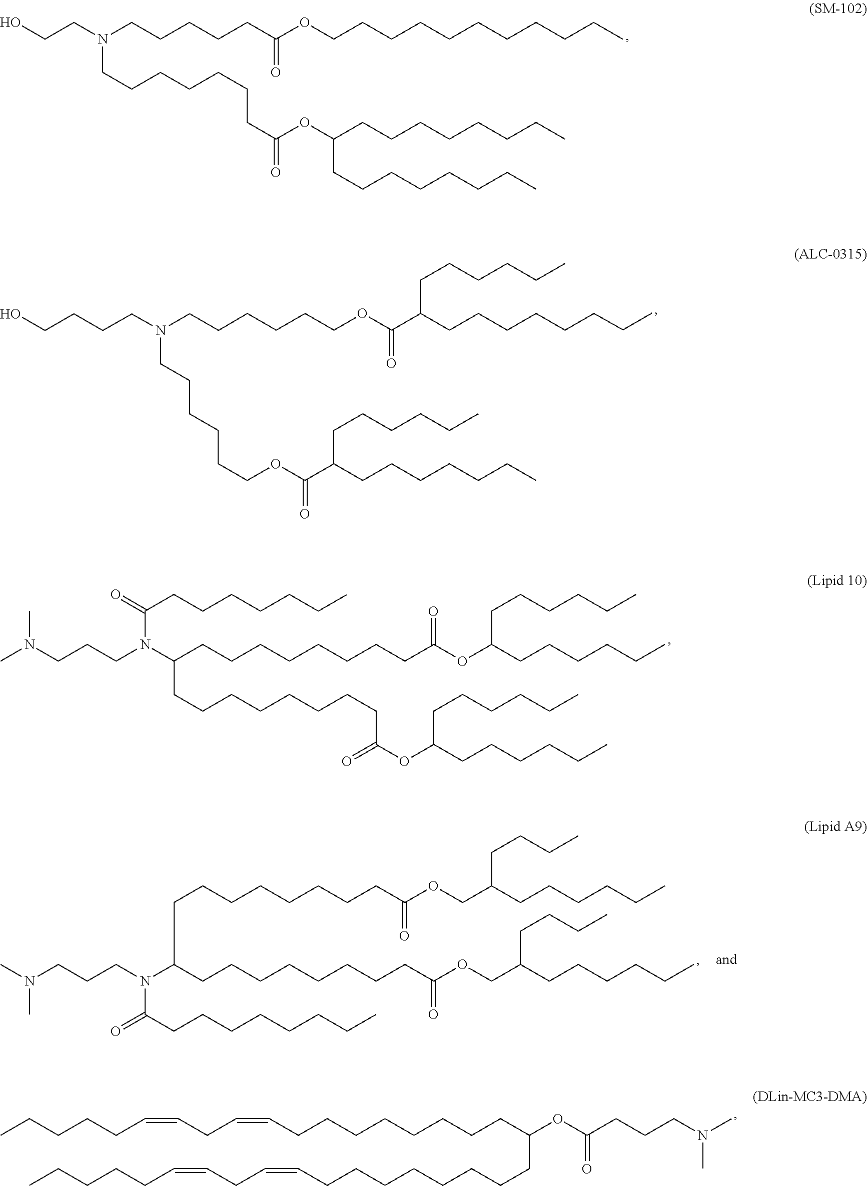

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 5.15.1.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US 2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 5.15.1.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, the entire contents of each of which are incorporated by reference herein for all purposes. Additional exemplary sterols include phytosterols, including those described in Eygeris et al. (2020), Nano Lett. 2020; 20(6):4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 5.15.1.4 Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350] (mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000] (mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-3000] (mPEG 3000 PE); 1,2-Diacyl-sn-Glycero-3-Phosphoethanolamine-N-[Methoxy(Polyethylene glycol)-5000] (mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

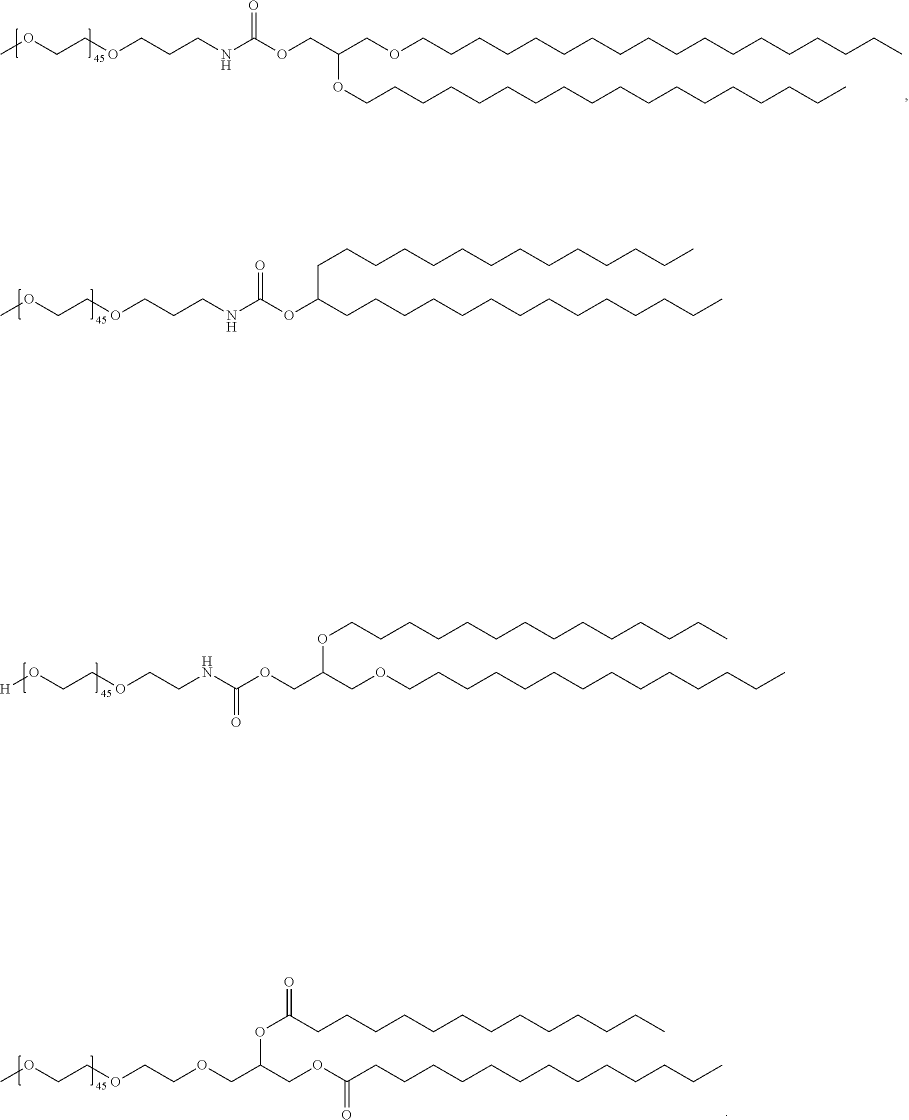

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), the entire contents of each of which are incorporated by reference herein for all purposes.

### 5.15.1.5 Percentages of Lipid Nanoformulation Components

In some embodiments, the lipid-based carrier (or lipid nanoformulation) comprises one of more of an agent described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises an agent described herein. The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises an agent described herein that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the agent may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises an agent described herein that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the agent may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises an agent described herein that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the agent may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises an agent described herein that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the agent may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises an agent described herein that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the agent may be at least 70%.

In some embodiments, the lipid-based carrier (or lipid nanoformulation) comprises (i)) an agent described herein; (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) an agent described herein; (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the agent can be varied as desired. For example, the total lipid components to the agent (mass or weight) ratio can be from about 10:1 to about 30:1. In some embodiments, the total lipid components to the agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the agent can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of an agent described herein that is encapsulated or otherwise associated with a lipid nanoformulation (e.g., liposome or LNP) after preparation, relative to the initial amount provided. The encapsulation efficiency is desirably high (e.g., at least 70%. 80%. 90%. 95%, close to 100%). The encapsulation efficiency may be measured, for example, by comparing the amount of the agent in a solution containing the liposome or LNP before and after breaking up the liposome or LNP with one or more organic solvents or detergents. An anion exchange resin may be used to measure the amount of free the agent in a solution. Fluorescence may be used to measure the amount of free the agent in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 5.16 Methods of Making Proteins

Proteins described herein (e.g., hIL-10R binding proteins, immunogenic proteins, IGIP proteins, fusion proteins, and protein conjugates) may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the hIL-10R binding protein, immunogenic protein, IGIP protein, fusion protein, and protein conjugate. Such general methods are common knowledge in the art. The expression vector typically contains an expression cassette that includes nucleic acid sequences capable of bringing about expression of the nucleic acid molecule encoding the protein of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the immunogenic protein. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

Proteins described herein (e.g., hIL-10R binding proteins, immunogenic proteins, IGIP proteins, fusion proteins, and protein conjugates) may be produced synthetically. Proteins described herein, and particularly the immunogenic proteins described herein, may be produced by using an egg-based manufacturing method.

In embodiments, the invention features methods of making the hIL-10R binding proteins, immunogenic proteins, fusion proteins, or protein conjugates described herein. The method includes (a) recombinantly expressing a hIL-10R binding protein, an immunogenic protein, an IGIP protein, a fusion protein, or protein conjugate described herein; (b) enriching, e.g., purifying, the hIL-10R binding protein, immunogenic protein, fusion protein, or protein conjugate described herein; (c) evaluating the hIL-10R binding protein, immunogenic protein, IGIP protein, fusion protein, or protein conjugate described herein described herein for the presence of a process impurity or contaminant, and (d) formulating the hIL-10R binding protein, immunogenic protein, IGIP protein, fusion protein, or protein conjugate as a pharmaceutical composition if the hIL-10R binding protein, immunogenic protein, IGIP protein, fusion protein, or protein conjugate meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.17 Methods of Making Nucleic Acid Molecules

Nucleic acid molecules described herein can be generated using common methods known in the art (e.g., chemical synthesis).

In some embodiments, the nucleic acid molecule is modified or varied (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.18 Nucleic Acid Molecules, Vectors, Host Cells, & Carriers

In one aspect, provided herein are nucleic acid molecules (e.g., DNA, RNA) encoding any protein described herein, e.g., any one or more of a hIL-10R binding protein (or functional fragment and/or functional variant thereof), an immunogenic protein (or immunogenic fragment and/or immunogenic variant thereof), an IGIP (e.g., hIGIP) protein (e.g., or a functional fragment and/or a functional variant thereof); a fusion protein described herein, a conjugate described herein, etc. In some embodiments, the polynucleotide is a DNA polynucleotide or an RNA (e.g., mRNA or circular RNA) polynucleotide. In some embodiments, the polynucleotide is an mRNA polynucleotide.

In some embodiments, the nucleic acid molecule is codon optimized. As discussed above, codon optimization, may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In one aspect, provided herein are vectors comprising any nucleic acid molecule described herein, e.g., any one or more of a nucleic acid molecule encoding a hIL-10R binding protein (or a functional fragment and/or functional variant thereof), a nucleic acid molecule encoding an immunogenic protein (or an immunogenic fragment or variant thereof), a nucleic acid molecule encoding a fusion protein described herein, or a nucleic acid molecule encoding a conjugate described herein, a polycistronic nucleic acid molecule, etc. In some embodiments, the vector is a viral vector. In some embodiments, the vector is a non-viral vector (e.g., a plasmid). In some embodiments, the vector is a plasmid. In some embodiments, the vector is a minicircle.

In one aspect, provided herein are host cells comprising any protein, nucleic acid molecule described herein, vector, or carrier described herein.

In one aspect, provided herein are carriers comprising any protein, nucleic acid molecule, vector, or host cell described herein. Carriers are further described in § 5.15, and exemplary carriers include, but are not limited to, lipid-based carriers such as LNPs, liposomes, lipoplexes, or nanoliposomes. In some embodiments, the carrier is an LNP, e.g., an LNP described herein, e.g., an LNP described herein.

### 5.19 Adjuvants

Any agent described herein (e.g., a hIL-10R binding protein (or functional fragment and/or functional variant thereof), an immunogenic protein (or immunogenic fragment and/or immunogenic variant thereof), an IGIP (e.g., hIGIP) protein (e.g., or a functional fragment and/or a functional variant thereof); a fusion protein described herein, a conjugate described herein, etc.) or composition described herein (e.g., combination composition, vaccine booster composition, pharmaceutical composition, etc.) may be co-administered with one or more adjuvants or comprise one or more adjuvants. Adjuvants are known in the art to further increase the immune response to an immunogen. General categories of adjuvants include, but are not limited to, inorganic adjuvants, small molecule adjuvants, oil in water emulsions, lipids or polymers, peptides or peptidoglycans, carbohydrates or polysaccharides, RNA-based adjuvants, DNA-based adjuvants, viral particles, bacterial adjuvants, inorganic nanoparticles, and multi-component adjuvants. Examples of adjuvants include, but are not limited to, aluminum salts such as aluminum hydroxide and/or aluminum phosphate; oil-emulsion compositions (or oil-in-water compositions), including squalene-water emulsions, such as MF59 (see, e.g., WO90/14837), MF59, ASO3, and Montanide; saponin formulations, such as for example QS21 and Immunostimulating Complexes (ISCOMS) (see, e.g., U.S. Pat. No. 5,057,540; WO90/03184, WO96/11711, WO2004/004762, WO2005/002620, the entire contents of each of which is incorporated by reference herein for all purposes); protamine or a protamine salt (e.g., protamine sulfate); calcium salt; bacterial or microbial derivatives, examples of which include monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g., antibodies or fragments thereof (e.g., directed against the immunogen itself or CD1a, CD3, CD7, CD80) and ligands to receptors (e.g., CD40L, GMCSF, GCSF, etc.).

Exemplary RNA-based adjuvants include, but are not limited to, Poly IC, Poly IC:LC, hairpin RNAs, e.g., with a 5′PPP containing sequence, viral sequences, polyU containing sequences, dsRNA, natural or synthetic immunostimulatory RNA sequences, nucleic acids analogs, optionally cyclic GMP-AMP or a cyclic dinucleotide such as cyclic di-GMP, and immunostimulatory base analogs, e.g., C8-substitued or an N7,C8-disubstituted guanine ribonucleotide. Exemplary DNA-based adjuvants, include, but are not limited to, CpGs, dsDNA, or natural or synthetic immunostimulatory DNA sequences. Exemplary bacteria-based adjuvants include, but are not limited, to bacterial adjuvant is flagellin, LPS, or a bacterial toxin, e.g., enterotoxins, heat-labile toxins, and Cholera toxins. Exemplary carbohydrate or polysaccharide adjuvants include, but are not limited to, dextran (branched microbial polysaccharide), dextran-sulfate, Lentinan, zymosan, Betaglucan, Deltin, Mannan, and Chitin. Exemplary small molecule adjuvants, include, but are not limited to, imiquimod, resiquimod, and gardiquimod. Exemplary lipid or polymer adjuvants, include, but are not limited to, polymeric nanoparticles (e.g., PLGA, PLG, PLA, PGA, or PHB), liposomes (e.g., Virosomes and CAFO1), LNPs or a component thereof, lipopolysaccharide (LPS) (e.g., monophosphoryl lipid A (MPLA) or glucopyranosyl Lipid A (GLA)), lipopeptides (e.g., Pam2 (Pam2CSK4) or Pam3 (Pam3CSK4)), and glycolipid (e.g., trehalose dimycolate). Exemplary peptides or peptidoglycan include, but are not limited to, N-acetyl-muramyl-L-alanyl-D-isoglutamine (MDP), flagellin-fusion protein, mannose-binding lectin (MBL), cytokines, and chemokine. Exemplary inorganic nanoparticle adjuvants, include, but are not limited to, gold nanorods, silica-based nanoparticles (e.g., mesoporous silica nanoparticles (MSN)). Exemplary multicomponent adjuvants include, but are not limited to, ASO1, ASO3, ASO4, Complete Freunds Adjuvant, and CAFO1.

### 5.20 Pharmaceutical Compositions

In one aspect, provided herein are pharmaceutical compositions comprising any agent described herein, protein described herein (including fusion protein or conjugates), nucleic acid molecule described herein, vector described herein, composition described herein, carrier described herein, and/or host cell described herein (e.g., a hIL-10R binding agent; a hIL-10R binding protein (or a functional fragment and/or functional variant thereof) (e.g., described herein, see, e.g., § 5.2) (or a nucleic acid molecule encoding the hIL-10R binding protein (or the functional fragment and/or functional variant thereof) (e.g., described herein, see, e.g., § 5.4)); an IGIP (e.g., hIGIP) protein (e.g., or a functional fragment and/or a functional variant thereof) (e.g., described herein, see, e.g., § 5.7) (or a fusion protein or conjugate thereof) (or the nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof); an immunogenic protein (or an immunogenic fragment or variant thereof) (e.g., described herein, see, e.g., § 5.5) (or a nucleic acid molecule encoding the immunogenic protein (or the immunogenic fragment or variant thereof) (e.g., described herein, see, e.g., § 5.6)), a polycistronic nucleic acid molecule (e.g., described herein (see, e.g., § 5.11)), a combination composition (e.g., described herein, see, e.g., § 5.12)); a vaccine composition (e.g., described herein, see, e.g., § 5.13)); a vector described herein (e.g., described herein, see, e.g., § 5.14)); a carrier described herein (e.g., described herein, see, e.g., § 5.15)), a host cell described herein (e.g., described herein, see, e.g., § 5.18))); and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing any agent described herein, protein described herein (including fusion protein or conjugates), nucleic acid molecule described herein, vector described herein, composition described herein, carrier described herein, and/or host cell described herein (e.g., a hIL-10R binding agent; a hIL-10R binding protein (or a functional fragment and/or functional variant thereof) (e.g., described herein, see, e.g., § 5.2) (or a nucleic acid molecule encoding the hIL-10R binding protein (or the functional fragment and/or functional variant thereof) (e.g., described herein, see, e.g., § 5.4)); an IGIP (e.g., hIGIP) protein (e.g., or a functional fragment and/or a functional variant thereof) (e.g., described herein, see, e.g., § 5.7) (or a fusion protein or conjugate thereof) (or the nucleic acid molecule encoding the IGIP (e.g., hIGIP) protein (e.g., or the functional fragment or the functional variant thereof)) (e.g., described herein, see, e.g., § 5.8))) (or a fusion protein or conjugate thereof); an immunogenic protein (or an immunogenic fragment or variant thereof) (e.g., described herein, see, e.g., § 5.5) (or a nucleic acid molecule encoding the immunogenic protein (or the immunogenic fragment or variant thereof) (e.g., described herein, see, e.g., § 5.6)), a polycistronic nucleic acid molecule (e.g., described herein (see, e.g., § 5.11)), a combination composition (e.g., described herein, see, e.g., § 5.12)); a vaccine composition (e.g., described herein, see, e.g., § 5.13)); a vector described herein (e.g., described herein, see, e.g., § 5.14)); a carrier described herein (e.g., described herein, see, e.g., § 5.15)), a host cell described herein (e.g., described herein, see, e.g., § 5.18))); and formulating it into a pharmaceutically acceptable composition by the addition of one or more pharmaceutically acceptable excipient.

Also provided herein are pharmaceutical compositions comprising an agent (e.g., a hIL-10R binding agent described herein) described herein, a protein described herein (e.g., a hIL-10R binding protein described herein, an immunogenic protein described herein, an IGIP protein described herein, a fusion protein described herein, a protein conjugate described herein), a polynucleotide described herein, a vector described herein, a host cell described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like. See, e.g., Chun, S., Daheshia, M., Lee, S., Eo, S. K., & Rouse, B. T. (1999). Distribution fate and mechanism of immune modulation following mucosal delivery of plasmid DNA encoding IL-10. Journal of immunology (Baltimore, Md.: 1950), 163(5), 2393-2402, the full contents of which is incorporated by reference herein for all purposes.

In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

In one embodiment, the pharmaceutical composition is formulated for mucosal administration. In one embodiment, the pharmaceutical composition is formulated for intranasal administration. In one embodiment, the pharmaceutical composition is formulated for inhalation. In one embodiment, the pharmaceutical composition is formulated for administration into the ear (or a specific compartment thereof (e.g., the middle ear, inner ear, etc.). In one embodiment, the pharmaceutical composition is formulated for into the ear (or a specific compartment thereof (e.g., the middle ear, inner ear, etc.) e.g., in the form of ear drops.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.21 Methods of Use

Provided herein are various methods of utilizing any agent described herein, protein described herein (including fusion proteins and conjugates), nucleic acid molecule described herein, vector described herein, composition described herein, carrier described herein, host cell described herein, and/or pharmaceutical composition described herein described herein (e.g., a hIL-10R binding agent (e.g., described herein, see, e.g., § 5.2); a hIL-10R binding protein (or a functional fragment and/or functional variant thereof) (e.g., described herein, see, e.g., § 5.2) (or a nucleic acid molecule encoding the hIL-10R binding protein (or the functional fragment and/or functional variant thereof) (e.g., described herein, see, e.g., § 5.4)); a fusion protein described herein, a conjugate described herein, an immunogenic protein (or an immunogenic fragment or variant thereof) (e.g., described herein, see, e.g., § 5.5) (or a nucleic acid molecule encoding the immunogenic protein (or the immunogenic fragment or variant thereof) (e.g., described herein, see, e.g., § 5.6)), an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)), a polycistronic nucleic acid molecule (e.g., described herein (see, e.g., § 5.11)), a combination composition (e.g., described herein, see, e.g., § 5.12)); a vaccine composition (e.g., described herein, see, e.g., § 5.13)); a vector described herein (e.g., described herein, see, e.g., § 5.14)); a carrier described herein (e.g., described herein, see, e.g., § 5.15)), a host cell described herein (e.g., described herein, see, e.g., § 5.18), and/or a pharmaceutical composition (e.g., described herein, see, e.g., § 5.20))).

The dosage of any of the foregoing, to be administered to a subject in accordance with any of the methods described herein can be determined in accordance with standard techniques known to those of ordinary skill in the art, including the route of administration, the age and weight of the subject, and the type (if any) adjuvant that is used. In some embodiments, the agent is administered to the subject in a therapeutically effective amount.

For example, in some embodiments, the hIL-10R binding protein can be administered to the subject at a dose of from about 5 μg/kg-160 μg/kg, 10 μg/kg-160 μg/kg, 20 μg/kg-160 μg/kg, 30 μg/kg-160 μg/kg, 40 μg/kg-160 μg/kg, 50 μg/kg-160 μg/kg, 60 μg/kg-160 μg/kg, 70 μg/kg-160 μg/kg, 80 μg/kg-160 μg/kg, 90 μg/kg-160 μg/kg, 100 μg/kg-160 μg/kg, 110 μg/kg-160 μg/kg, 120 μg/kg-160 μg/kg, 130 μg/kg-160 μg/kg, 140 μg/kg-160 μg/kg, or 150 μg/kg-160 μg/kg. In some embodiments, the hIL-10R binding protein can be administered to the subject at a dose of from about 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, or 1600 μg/kg.

In some aspects, the methods described herein comprise administering one or more of the foregoing to a subject. Exemplary subjects include mammals, e.g., humans, non-human mammals, e.g., non-human primates. In some embodiments, the subject is a human.

In some embodiments, the subject is a vertebrate animal (e.g., mammal, bird, fish, reptile, or amphibian). In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age. In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

### 5.21.1 Methods of Vaccination

Provided herein are, inter alia, various methods of vaccinating subjects (e.g., human subjects). Vaccinations include, e.g., vaccination against an infective agent (e.g., a pathogen (e.g., a virus, bacteria, fungus, protozoa)) or a tumor. In some embodiments, the vaccination is against a tumor. In some embodiments, the vaccination is against a pathogen. In some embodiments, the pathogen is a virus, bacteria, fungus, yeast, or protozoa.

In some embodiments, the pathogen is a virus. Exemplary viruses include, but are not limited to, coronaviruses (e.g., SARS-CoV-2, SARS-CoV, MERS-CoV virus), influenza viruses (e.g., influenza A, influenza B), respiratory syncytial viruses (RSV), rhinoviruses, parvoviruses (e.g., parvovirus B19), parainfluenza viruses, adenoviruses, varicella zoster viruses, papillomaviruses, yellow fever viruses, rabies lyssaviruses, variola viruses (e.g., variola major virus, variola minor virus, small pox virus, monkey pox virus), hepatitis B viruses, varicella viruses, tick-borne encephalitis (TBE) viruses, Japanese encephalitis viruses, rotaviruses, mumps viruses, rubella viruses, measles viruses, polioviruses, dengue viruses, sapoviruses, noroviruses, enteroviruses, and astroviruses. In some embodiments, the virus is a respiratory virus. In some embodiments, the virus is a coronavirus (e.g., a SARS-CoV-2 virus, a SARS-CoV virus, a MERS-CoV virus), an influenza virus (e.g., influenza A, influenza B), a respiratory syncytial virus (RSV), a rhinovirus, a parvovirus (e.g., a parvovirus B19), a parainfluenza virus, or an adenovirus. In some embodiments, the virus is a rotavirus, an adenovirus, a sapovirus, a norovirus, an enterovirus, or an astrovirus.

In some embodiments, the pathogen is a bacterium. Exemplary bacteria include, but are not limited to, Streptococcus (e.g., Streptococcus pneumoniae), Neisseria (e.g., Neisseria meningitidis) (e.g., serogroups A, B, C, W, and Y), Salmonella (e.g., Salmonella Typhi), Vibrio (e.g., Vibrio cholerae, Vibrio parahaemolyticus), Clostridium (e.g., Clostridium tetani, Clostridium botulinum, Clostridium difficile), Haemophilus (e.g., Haemophilus influenzae), Bacillus (e.g., Bacillus anthracis), Mycobacterium (e.g., Mycobacterium tuberculosis), Campylobacter (e.g., Campylobacter jejuni), Shigella, Listeria (e.g., Listeria monocytogenes), Escherichia (e.g., Escherichia coli), Giardia (e.g., Giardia lamblia), Helicobacter (e.g., Heliobacter pylori), Yersinia (e.g., Yersinia enterocolitica), Cryptosporidium (e.g., Cryptosoridium parvum), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

In some embodiments, the pathogen is a protozoan. Exemplary protozoa include, but are not limited to, Leishmania (e.g., Leishmania major), Toxoplasma (e.g., Toxoplasma gondii), Plasmodium (e.g., Plasmodium falciparum), Leishmania (e.g., Leishmania infantum), Eimeria, Theileria (e.g., Theileria parva, Theileria annulate), Babesia (e.g., Babesia bovis, Babesia bigemina), Tritrichomonas (e.g., Tritrichomonas foetus), Giardia (e.g., Giardia lamblia), Sarcocystis (e.g., Sarcocystis neurona), Neospora (e.g., Neospora caninum), Entamoeba (e.g., Entamoeba Dispar, Entamoeba histolytica).

In some embodiments, the pathogen is a fungus. Exemplary fungi include, but are not limited to, Candidisis, Aspergillusis, Paracoccidioidomycosis, Blastomycosis, Coccidiomycosis, Histoplasmosis, Cryptococcusis, and Pneumocystosis.

In some embodiments, the pathogen is a mucosal (e.g., respiratory mucosa, oral mucosa, gastrointestinal mucosa, or urogenital mucosa) pathogen. In some embodiments, the mucosal pathogen is a virus, bacteria, protozoa, or fungus. In some embodiments, the mucosal pathogen is a respiratory pathogen, an oral pathogen, a gastrointestinal pathogen, or a urogenital pathogen.

Exemplary mucosal pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, rotaviruses, adenoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), Entamoeba (e.g., Entamoeba Dispar, Entamoeba histolytica), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus), and Candidiasis.

Exemplary respiratory pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, and adenoviruses.

Exemplary gastrointestinal pathogens include, but are not limited to, adenoviruses, sapoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), and Entamoeba (e.g., Entamoeba Dispar, Entamoeba histolytica).

Exemplary urogenital pathogens include, but are not limited to, Candidiasis, Escherichia (e.g., Escherichia coli), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

The various methods of vaccination described herein include methods of simultaneous vaccination against more than pathogen (i.e., a plurality of pathogens). For example, where methods of vaccinating a subject are described herein, further provided are methods of vaccinating the subject against more a than one (e.g., 2, 3, 4, 5, 6, 7, 8, 9, or 10) different pathogens. Further, where methods of vaccinating a subject are described herein, further provided are methods of vaccinating a subject against more than one (e.g., 2, 3, 4, 5, 6, 7, 8, 9, or 10) different strains of the same pathogen.

### 5.21.1.1 Methods of Vaccinating a Subject

In one aspect, provided herein are methods of vaccinating a subject (e.g., against an infective agent (e.g., a pathogen) or a tumor) comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccinate the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering to the subject (b) in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby vaccinate the subject

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering (a) in combination with (b) to the subject, to thereby vaccinate the subject.

Provided herein is a combination composition described herein or combination therapy described herein for use in a method of vaccinating a subject, the method comprising administering to the subject the combination composition described herein, or the combination therapy described herein, to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of vaccinating a subject, the method comprising administering (a) in combination with (b) to the subject, to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

Provided herein is a combination composition described herein or combination therapy described herein for the manufacture of a medicament for use in a method of vaccinating a subject.

In some embodiments, the immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are methods of vaccinating a subject (e.g., against an infective agent (e.g., a pathogen) or a tumor) comprising (a) first administering to the subject at least a first dose of an immunogen (e.g., from the infective agent (e.g., the pathogen) or the tumor) (e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is (b) a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject (e.g., against an infective agent (e.g., a pathogen) or a tumor), the method comprising (a) first administering to the subject at least a first dose of an immunogen (e.g., from the infective agent (e.g., the pathogen) or the tumor) (e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), and (b) thereafter (e.g., as a booster) administering to the subject the hIL-10R binding agent, to thereby vaccine the subject

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising (a) first administering to the subject at least a first dose of an immunogen (e.g., from the infective agent (e.g., the pathogen) or the tumor) (e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of vaccinating a subject, the method comprising (a) first administering the immunogen (a) to the subject, and (b) thereafter (e.g., as a booster) administering the hIL-10R binding agent (b) to the subject, to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby vaccine the subject.

In some embodiments, the immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, thereafter is from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month. In some embodiments, thereafter is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

In some embodiments, the method further comprises administering an immunogen to the subject in combination with the administration of the hIL-10R binding agent (e.g., (b)(i)-(vi)). In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a). In some embodiments, the method further comprises administering the immunogen (e.g., (a)(i)-(vi)) to the subject in combination with the administration of the hIL-10R binding agent (e.g., (b)(i)-(vi)).

In some embodiments, the hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject. In some embodiments, the boost portion of the regimen comprises the administration of an immunogen and the hIL-10R binding agent (i.e., (b)(i)-(vi)). In some embodiments, the boost portion of the regimen comprises the administration of the immunogen (e.g., (a)(i)-(vi)) and the hIL-10R binding agent (i.e., (b)(i)-(vi)).

For the sake of clarity it is to be understood that the following embodiments are applicable to any of the foregoing aspects.

(a) and (b) can be administered to the subject by any route (e.g., described herein, see, e.g., § 5.20). In some embodiments the first administering of (a) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments, the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments the first administering of (a) comprises parenteral administration (e.g., intramuscular, intradermal, subcutaneous, transcutaneous, or mucosal administration, (e.g., inhalation, intranasal, oral)); and the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))).

Non-limiting embodiments include parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like. In some embodiments, the first administering of (a) comprises intramuscular, subcutaneous, or intranasal administration. In some embodiments, the thereafter administering of (b) comprises intramuscular, subcutaneous, or intranasal administration. In some embodiments, the first administering of (a) comprises intramuscular, subcutaneous, or intranasal administration and the thereafter administering of (b) comprises intramuscular, subcutaneous, or intranasal administration. In some embodiments, the first administering of (a) comprises intramuscular or subcutaneous administration. In some embodiments, the thereafter administering of (b) comprises intranasal administration. In some embodiments, the first administering of (a) comprises intramuscular or subcutaneous administration and the thereafter administering of (b) comprises intranasal administration. In some embodiments, the first administering of (a) comprises intranasal administration and the thereafter administering of (b) comprises intranasal administration.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the immunogen (e.g., (a)(i)-(vi)) is administered intramuscularly, subcutaneously, or mucosally (e.g., intranasally). In some embodiments, the immunogen (e.g., (a)(i)-(vi)) is administered intramuscularly or subcutaneously. In some embodiments, the hIL-10R binding agent is administered intranasally and the immunogen (e.g., (a)(i)-(vi)) is administered intramuscularly or subcutaneously.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or or after administration of the hIL-10R binding agent (e.g., (b)(i)-(vi)).

### 5.21.1.2 Methods of Vaccinating a Subject Utilizing an mRNA Vaccine

In one aspect, provided herein are methods of vaccinating a subject (e.g., against an infective agent (e.g., a pathogen) or a tumor) comprising administering to the subject (a) an mRNA (e.g., described herein) vaccine against an infective agent (e.g., pathogen) or tumor; in combination with (b) a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is (b) a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering to the subject (b) in combination with (a) an mRNA (e.g., described herein) vaccine against an infective agent (e.g., pathogen) or tumor, to thereby vaccine the subject.

Provided herein is a combination of (a) an mRNA (e.g., described herein) vaccine against an infective agent (e.g., pathogen) or tumor and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering (a) and (b) to the subject, to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an mRNA (e.g., described herein) vaccine against an infective agent (e.g., pathogen) or tumor, for use in a method of vaccinating a subject, the method comprising administering to the subject (a) in combination with (b), to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) an mRNA (e.g., described herein) vaccine against an infective agent (e.g., pathogen) or tumor, to thereby vaccinate the subject.

In some embodiments, the mRNA vaccine comprises an mRNA molecule encoding at least one immunogen. In some embodiments, the mRNA molecule is contained within a vector (e.g., described herein). In some embodiments, the mRNA molecule (or the vector) is contained within a carrier (e.g., described herein).

In some embodiments, the hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the mRNA vaccine and the hIL-10R binding agent (e.g., (b)(i)-(vi)) are administered to the subject as part of a prime-boost regimen. In some embodiments, the mRNA vaccine is administered to the subject as a prime vaccine and the hIL-10R binding agent (e.g., (b)(i)-(vi)) is administered to the subject as a prime vaccine. In some embodiments, the mRNA vaccine is administered to the subject as a prime vaccine and the hIL-10R binding agent (e.g., (b)(i)-(vi)) is subsequently administered to the subject as a booster vaccine.

In some embodiments, the mRNA vaccine is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen (e.g., immunogenic protein (or a nucleic acid molecule encoding the immunogenic protein). In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are methods of vaccinating a subject (e.g., a human subject) comprising (a) first administering to the subject an mRNA (e.g., described herein) vaccine against an infective agent (e.g., pathogen) or tumor, and (b) thereafter administering to the human subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby vaccinate the subject.

Provided herein is (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject (e.g., against an infective agent (e.g., a pathogen) or a tumor), the method comprising (a) first administering to the subject (a) an mRNA (e.g., described herein) vaccine against an infective agent (e.g., pathogen) or tumor; and (b) thereafter (e.g., as a booster) administering (b) to the subject, to thereby vaccine the subject.

Provided herein is a combination of (a) an mRNA (e.g., described herein) vaccine against an infective agent (e.g., pathogen) or tumor and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising (a) first administering to the subject (a) an mRNA (e.g., described herein) vaccine against an infective agent (e.g., pathogen) or tumor, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an mRNA (e.g., described herein) vaccine against an infective agent (e.g., pathogen) or tumor for the manufacture of a medicament for use in a method of vaccinating a subject, the method comprising (a) first administering to the subject (a) an mRNA (e.g., described herein) vaccine against an infective agent (e.g., pathogen) or tumor, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) an mRNA (e.g., described herein) vaccine against an infective agent (e.g., pathogen) or tumor, wherein the method comprises (a) first administering to the subject (a) an mRNA (e.g., described herein) vaccine against an infective agent (e.g., pathogen) or tumor, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

In some embodiments, the mRNA vaccine comprises an mRNA molecule encoding at least one immunogen. In some embodiments, the mRNA molecule is contained within a vector (e.g., described herein). In some embodiments, the mRNA molecule (or the vector) is contained within a carrier (e.g., described herein).

In some embodiments, the human subject a hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v). In some embodiments, (b) increases the production of nasal IgA in the human subject.

In some embodiments, thereafter is from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month. In some embodiments, thereafter is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

In some embodiments, the method further comprises administering an immunogen to the subject in combination with the administration of the hIL-10R binding agent (e.g., (b)(i)-(vi)). In some embodiments, the immunogen is the same as the encoded immunogen administered in (a). In some embodiments, the immunogen is different from the encoded immunogen administered in (a). In some embodiments, the immunogen is from the same infectious agent as the encoded immunogen administered in (a). In some embodiments, the immunogen is a variant of the encoded immunogen administered in (a).

In some embodiments, the hIL-10R binding agent (e.g., (b)(i)-(vi)) is administered as a booster in a prime-boost regimen, wherein the prime portion of the regimen comprises administration of the mRNA vaccine to the subject. In some embodiments, the boost portion of the regimen comprises the administration of an immunogen and the hIL-10R binding agent (i.e., (b)(i)-(vi)). In some embodiments, the boost portion of the regimen comprises the administration of the mRNA administered in (a) and the hIL-10R binding agent (i.e., (b)(i)-(vi)).

For the sake of clarity it is to be understood that the following embodiments are applicable to any of the foregoing aspects.

(a) and (b) can be administered to the subject by any route (e.g., described herein, see, e.g., § 5.20). In some embodiments the first administering of (a) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments, the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments the first administering of (a) comprises parenteral administration (e.g., intramuscular, intradermal, subcutaneous, transcutaneous, or mucosal administration, (e.g., inhalation, intranasal, oral)); and the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))).

Non-limiting embodiments include parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like. In some embodiments, the first administering of (a) comprises intramuscular, subcutaneous, or intranasal administration. In some embodiments, the thereafter administering of (b) comprises intramuscular, subcutaneous, or intranasal administration. In some embodiments, the first administering of (a) comprises intramuscular, subcutaneous, or intranasal administration and the thereafter administering of (b) comprises intramuscular, subcutaneous, or intranasal administration. In some embodiments, the first administering of (a) comprises intramuscular or subcutaneous administration. In some embodiments, the thereafter administering of (b) comprises intranasal administration. In some embodiments, the first administering of (a) comprises intramuscular or subcutaneous administration and the thereafter administering of (b) comprises intranasal administration. In some embodiments, the first administering of (a) comprises intranasal administration and the thereafter administering of (b) comprises intranasal administration.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent is administered intranasally and the at least a first dose of the immunogen (e.g., (a)(i)-(vi)) is administered intramuscularly or subcutaneously.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or or after administration of the hIL-10R binding agent (e.g., (b)(i)-(vi)).

### 5.21.1.3 Methods of Vaccinating a Subject Against SARS-CoV-2

In one aspect, provided herein are methods of vaccinating a subject (e.g., a human subject) comprising administering to the subject (a) a SARS-CoV-2 mRNA vaccine formulated in an LNP, in combination with (b) a hIL-10R binding agent e.g., comprising (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby vaccine the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering to the subject (a) in combination with (a) a SARS-CoV-2 mRNA vaccine formulated in an LNP to thereby vaccinate the subject.

Provided herein is a combination of (a) a SARS-CoV-2 mRNA vaccine formulated in an LNP and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising administering (a) and (b) to the subject, to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) a SARS-CoV-2 mRNA vaccine formulated in an LNP for use in a method of vaccinating a subject, the method comprising administering (a) and (b) to the subject, to thereby vaccinate the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) a SARS-CoV-2 mRNA vaccine formulated in an LNP.

In some embodiments, the hIL-10R binding agent comprises (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v). In some embodiments, the nucleic acid molecule is an mRNA. In some embodiments, (b) increases the production of nasal IgA in the human subject.

In some embodiments, the mRNA vaccine and the hIL-10R binding agent (e.g., (b)(i)-(vi)) are administered to the subject as part of a prime-boost regimen. In some embodiments, the mRNA vaccine is administered to the subject as a prime vaccine and the hIL-10R binding agent (e.g., (b)(i)-(vi)) is administered to the subject as a prime vaccine. In some embodiments, the mRNA vaccine is administered to the subject as a prime vaccine and the hIL-10R binding agent (e.g., (b)(i)-(vi)) is subsequently administered to the subject as a booster vaccine.

In some embodiments, the mRNA vaccine is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen (e.g., immunogenic protein (or a nucleic acid molecule encoding the immunogenic protein) (e.g., a SARS-CoV-2 immunogen). In some embodiments, the immunogen (e.g., a SARS-CoV-2 immunogen) is the same as the immunogen (e.g., SARS-CoV-2 immunogen) administered in (a). In some embodiments, the immunogen (e.g., a SARS-CoV-2 immunogen) is different from the immunogen (e.g., SARS-CoV-2 immunogen) administered in (a). In some embodiments, the immunogen (e.g., a SARS-CoV-2 immunogen) is from the same infectious agent as the immunogen (e.g., SARS-CoV-2 immunogen) administered in (a). In some embodiments, the immunogen (e.g., a SARS-CoV-2 immunogen) is a variant of the immunogen administered in (a) (e.g., SARS-CoV-2 immunogen).

In one aspect, provided herein are methods of vaccinating a subject (e.g., a human subject) comprising (a) first administering to the human subject a SARS-CoV-2 mRNA vaccine formulated in an LNP, and (b) thereafter administering to the human subject a hIL-10R binding agent e.g., comprising (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby vaccine the subject.

Provided herein is (b) a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject (e.g., against an infective agent (e.g., a pathogen) or a tumor), the method comprising (a) first administering to the subject (a) an mRNA (e.g., described herein) vaccine against an infective agent (e.g., pathogen) or tumor, and (b) thereafter (e.g., as a booster) administering to the subject the hIL-10R binding agent (b), to thereby vaccine the subject.

Provided herein is a combination of (a) a SARS-CoV-2 mRNA vaccine formulated in an LNP and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of vaccinating a subject, the method comprising (a) first administering to the subject (a) a SARS-CoV-2 mRNA vaccine formulated in an LNP, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) a SARS-CoV-2 mRNA vaccine formulated in an LNP for use in a method of vaccinating a subject, the method comprising (a) first administering to the subject (a) a SARS-CoV-2 mRNA vaccine formulated in an LNP, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of vaccinating a subject in need thereof, wherein the medicament is administered to the subject in combination with (a) a SARS-CoV-2 mRNA vaccine formulated in an LNP, wherein the method comprises (a) first administering to the subject (a) a SARS-CoV-2 mRNA vaccine formulated in an LNP, and (b) thereafter (e.g., as a booster) administering to the subject a hIL-10R binding agent (e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby vaccine the subject.

In some embodiments, the hIL-10R binding agent comprises (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v). In some embodiments, the nucleic acid molecule is an mRNA. In some embodiments, (b) increases the production of nasal IgA in the human subject.

In some embodiments, thereafter is from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month. In some embodiments, thereafter is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

In some embodiments, the method further comprises administering an immunogen (e.g., a SARS-CoV-2 immunogen) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the SARS-CoV-2 mRNA vaccine to the subject; and the boost portion of the regimen comprises the administration of the immunogen (e.g., the SARS-CoV-2 immunogen) and the hIL-10R binding agent (e.g., (b)(i)-(vi)).

For the sake of clarity it is to be understood that the following embodiments are applicable to any of the foregoing aspects.

(a) and (b) can be administered to the subject by any route (e.g., described herein, see, e.g., § 5.20). In some embodiments the first administering of (a) comprises parenteral administration (e.g., intramuscular, intradermal, subcutaneous, transcutaneous, or mucosal administration, (e.g., inhalation, intranasal, oral)). In some embodiments, the thereafter administering of (b) comprises parenteral administration (e.g., intramuscular, intradermal, subcutaneous, transcutaneous, or mucosal administration, (e.g., inhalation, intranasal, oral)). In some embodiments the first administering of (a) comprises parenteral administration (e.g., intramuscular, intradermal, subcutaneous, transcutaneous, or mucosal administration, (e.g., inhalation, intranasal, oral)); and the thereafter administering of (b) comprises parenteral administration (e.g., intramuscular, intradermal, subcutaneous, transcutaneous, or mucosal administration, (e.g., inhalation, intranasal, oral))._Non-limiting embodiments include parenteral administration, such as intramuscular, intradermal, subcutaneous, transcutaneous, or mucosal administration, e.g., inhalation, intranasal, oral, and the like. In some embodiments, the first administering of (a) comprises intramuscular, subcutaneous, or intranasal administration. In some embodiments, the thereafter administering of (b) comprises intramuscular, subcutaneous, or intranasal administration. In some embodiments, the first administering of (a) comprises intramuscular, subcutaneous, or intranasal administration and the thereafter administering of (b) comprises intramuscular, subcutaneous, or intranasal administration. In some embodiments, the first administering of (a) comprises intramuscular or subcutaneous administration. In some embodiments, the thereafter administering of (b) comprises intranasal administration. In some embodiments, the first administering of (a) comprises intramuscular or subcutaneous administration and the thereafter administering of (b) comprises intranasal administration.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the mRNA vaccine. In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent.

### 5.21.2 Methods of Ameliorating, Treating, or Preventing Infections

Provided herein are, inter alia, various methods of preventing, treating, or ameliorating an infection in a subject (e.g., a human subject). Infections, include, e.g., viral infections, bacterial infections, fungal infections, yeast infections, and protozoal infections. In some embodiments, the infection is a bacterial infection. In some embodiments, the infection is a viral infection. In some embodiments, the infection is a fungal infection. In some embodiments, the infection is a protozoal infection.

Methods described herein further include methods of preventing or treating more than one infection (e.g., a plurality of infections).

Exemplary viruses include, but are not limited to, coronaviruses (e.g., SARS-CoV-2 virus, SARS-CoV virus, MERS-CoV virus), influenza viruses (e.g., influenza A, influenza B), respiratory syncytial viruses (RSV), rhinoviruses, parvoviruses (e.g., parvovirus B19), parainfluenza viruses, adenoviruses, varicella zoster viruses, papillomaviruses, yellow fever viruses, rabies lyssaviruses, variola viruses (e.g., variola major virus, variola minor virus, small pox virus, monkey pox virus), hepatitis B viruses, varicella viruses, tick-borne encephalitis (TBE) viruses, Japanese encephalitis viruses, rotaviruses, mumps viruses, rubella viruses, measles viruses, polioviruses, dengue viruses, sapoviruses, noroviruses, enteroviruses, and astroviruses. In some embodiments, the virus is a respiratory virus. In some embodiments, the virus is a coronavirus (e.g., a SARS-CoV-2 virus, a SARS-CoV virus, a MERS-CoV virus), an influenza virus (e.g., influenza A, influenza B), a respiratory syncytial virus (RSV), a rhinovirus, a parvovirus (e.g., parvovirus B19), a parainfluenza virus, or an adenovirus. In some embodiments, the virus is a rotavirus, an adenovirus, a sapovirus, a norovirus, an enterovirus, or an astrovirus.

Exemplary bacteria include, but are not limited to, Streptococcus (e.g., Streptococcus pneumoniae), Neisseria (e.g., Neisseria meningitidis) (e.g., serogroups A, B, C, W, and Y), Salmonella (e.g., Salmonella Typhi), Vibrio (e.g., Vibrio cholerae, Vibrio parahaemolyticus), Clostridium (e.g., Clostridium tetani, Clostridium botulinum, Clostridium difficile), Haemophilus (e.g., Haemophilus influenzae), Bacillus (e.g., Bacillus anthracis), Mycobacterium (e.g., Mycobacterium tuberculosis), Campylobacter (e.g., Campylobacter jejuni), Shigella, Listeria (e.g., Listeria monocytogenes), Escherichia (e.g., Escherichia coli), Giardia (e.g., Giardia lamblia), Helicobacter (e.g., Heliobacter pylori), Yersinia (e.g., Yersinia enterocolitica), Cryptosporidium (e.g., Cryptosoridium parvum), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

Exemplary protozoans include, but are not limited to, Leishmania (e.g., Leishmania major), Toxoplasma (e.g., Toxoplasma gondii), Plasmodium (e.g., Plasmodium falciparum), Leishmania (e.g., Leishmania infantum), Eimeria, Theileria (e.g., Theileria parva, Theileria annulate), Babesia (e.g., Babesia bovis, Babesia bigemina), Tritrichomonas (e.g., Tritrichomonas foetus), Giardia (e.g., Giardia lamblia), Sarcocystis (e.g., Sarcocystis neurona), Neospora (e.g., Neospora caninum), Entamoeba (e.g., Entamoeba Dispar, Entamoeba histolytica).

Exemplary fungi include, but are not limited to, Candidisis, Aspergillusis, Paracoccidioidomycosis, Blastomycosis, Coccidiomycosis, Histoplasmosis, Cryptococcusis, and Pneumocystosis.

In some embodiments, the pathogen is a mucosal (e.g., respiratory mucosa, oral mucosa, gastrointestinal mucosa, or urogenital mucosa) pathogen. In some embodiments, the mucosal pathogen is a virus, bacteria, protozoa, or fungus. In some embodiments, the mucosal pathogen is a respiratory pathogen, an oral pathogen, a gastrointestinal pathogen, or a urogenital pathogen.

Exemplary mucosal pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, rotaviruses, adenoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), Entamoeba (e.g., Entamoeba Dispar, Entamoeba histolytica), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus), and Candidiasis.

Exemplary respiratory pathogens include, but are not limited to, coronaviruses (e.g., a SARS-CoV-2 virus, a SARS-CoV virus, a MERS-CoV virus), influenza viruses (e.g., influenza A, influenza B), respiratory syncytial viruses (RSV), rhinoviruses, parvoviruses (e.g., parvovirus B19), parainfluenza viruses, and adenoviruses.

Exemplary gastrointestinal pathogens include, but are not limited to, adenoviruses, sapoviruses, noroviruses, enteroviruses, astroviruses, Salmonella (e.g., Salmonella Typhi), Campylobacter (e.g., Campylobacter jejuni), Shigella, Listeria (e.g., Listeria monocytogenes), Vibrio (e.g., Vibrio cholerae, Vibrio parahaemolyticus), Escherichia (e.g., Escherichia coli), Giardia (e.g., Giardia lamblia), Clostridium (e.g., Clostridium tetani, Clostridium botulinum, Clostridium difficile), Helicobacter (e.g., Heliobacter pylori), Yersinia (e.g., Yersinia enterocolitica), and Cryptosporidium (e.g., Cryptosoridium parvum), and Entamoeba (e.g., Entamoeba Dispar, Entamoeba histolytica).

Exemplary urogenital pathogens include, but are not limited to, Candidiasis, Escherichia (e.g., Escherichia coli), Klebsiella (e.g., Klebsiella pneumoniae), Proteus (e.g., Proteus mirabilis), Enterococcus (e.g., Enterococcus faecalis) and Staphylococcus (e.g., Staphylococcus saprophyticus).

The various methods of preventing or treating an infection described herein include methods of simultaneous prevention or treatment from infection by more than one pathogen (i.e., a plurality of pathogens). For example, where methods of prevention or treatment of an infection in a subject are described herein, further provided are methods of preventing or treating more than one (e.g., 2, 3, 4, 5, 6, 7, 8, 9, or 10) infection in the subject, wherein each infection is of a different pathogen. Further, where methods of prevention or treatment of an infection in a subject are described herein, further provided are methods of preventing or treating more than one (e.g., 2, 3, 4, 5, 6, 7, 8, 9, or 10) infection in the subject, wherein each infection is of a different strain of the same.

### 5.21.2.1 Methods of Ameliorating, Treating, or Preventing an Infection

In one aspect, provided herein are methods of preventing, treating, or ameliorating an infection (e.g., of an infective agent) in a subject, the method comprising administering to the subject (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (b) is administered to the subject in an amount and for a time sufficient to prevent, treat, or ameliorate the infection in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of preventing, treating, or ameliorating an infection (e.g., of an infective agent) in a subject, the method comprising administering to the subject (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); in combination with (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (a) and/or (b) are administered to the subject in an amount and for a time sufficient to prevent, treat, or ameliorate the infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection in a subject, the method comprising administering to the subject (b) in combination with (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), to thereby prevent, treat, or ameliorate the infection in the subject.

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject, the method comprising administering (a) and (b) to the subject, to thereby prevent, treat, or ameliorate the infection the subject.

Provided herein is a combination composition described herein or combination therapy described herein for use in a method of preventing, treating, or ameliorating an infection a subject, the method comprising administering to the subject the combination composition described herein, or the combination therapy described herein, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of preventing, treating, or ameliorating an infection a subject, the method comprising administering (a) in combination with (b) to the subject, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an infection a subject in need thereof, wherein the medicament is administered to the subject in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

Provided herein is a combination composition described herein or combination therapy described herein for the manufacture of a medicament for use in a method of preventing, treating, or ameliorating an infection a subject.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are methods of preventing, treating, or ameliorating an infection (e.g., of an infective agent) in a subject, the method comprising (a) first administering to a subject at least a first dose of an (a) immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), and (b) thereafter (e.g., as a booster) administering to the subject a hIL-10R binding agent e.g., comprising (i) a (b) hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (a) and/or (b) are administered to the subject in an amount and for a time sufficient to prevent, treat, or ameliorate the infection in the subject.

Provided herein is (b) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, treating, or ameliorating an infection in a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a combination of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a use of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) for the manufacture of a medicament for use in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in a method of preventing, treating, or ameliorating an infection a subject in need thereof, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), wherein the method comprises (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the method further comprises administering the immunogen (e.g., (a)(i)-(vi)) to the subject in combination with the administration of the hIL-10R binding agent (e.g., (b)(i)-(vi)). In some embodiments, hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)).

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject. In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In one aspect, provided herein are methods of treating a human subject exposed to an infective agent, comprising administering to the subject (a) an immunogen (e.g., from the infective agent) (e.g., comprising, e.g., (i) an immunogenic protein (e.g., described herein) (or an immunogenic fragment or variant thereof) (or a fusion protein or conjugate thereof), (ii) a nucleic acid (e.g., RNA) molecule comprising a coding region encoding an immunogenic protein (e.g., described herein) (or an immunogenic fragment or variant thereof) (or a fusion protein or conjugate thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), in combination with (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

Provided herein is (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) for use in a method of treating a human subject exposed to an infective agent, the method comprising administering to the subject (a) in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby treat a human subject exposed to an infective agent.

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of treating a human subject exposed to an infective agent, the method comprising administering (a) and (b) to the subject, to thereby treat a human subject exposed to an infective agent.

Provided herein is a combination composition described herein or combination therapy described herein for use in a method of treating a human subject exposed to an infective agent, the method comprising administering to the subject the combination composition described herein, or the combination therapy described herein, to treat a human subject exposed to an infective agent.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of treating a human subject exposed to an infective agent, the method comprising administering (a) and (b) to the subject, to thereby treat a human subject exposed to an infective agent.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in a method of treating a human subject exposed to an infective agent, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby treat a human subject exposed to an infective agent.

Provided herein is a combination composition described herein or combination therapy described herein for the manufacture of a medicament for use in a method of treating a human subject exposed to an infective agent.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the infective agent is a virus. In some embodiments, the infective agent is a coronavirus (e.g., a SARS-CoV-2 virus, a SARS-CoV virus, or a MERS-CoV SARS-CoV-2 virus). In some embodiments, the infective agent is a SARS-CoV-2 virus.

In some embodiments, the subject has an acute infection with the infective agent. In some embodiments, the subject has a post viral syndrome (e.g., long Covid) from a previous acute viral infection.

In some embodiments, the immunogen is the same as a vaccine immunogen. In some embodiments, the subject has not previously had at least one vaccine dose against the infective agent. In some embodiments, the subject has previously had at least one vaccine dose against the infective agent.

In one aspect, provided herein are methods of preventing, treating, or ameliorating an infection (e.g., of an infective agent) in a subject, the method comprising administering to a subject that has received at least a first dose of a vaccine regimen against an infective agent (e.g., a pathogen) or a tumor (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (b) is administered to the subject in an amount and for a time sufficient to prevent, treat, or ameliorate the infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection in a subject that has received at least a first dose of a vaccine regimen against an infective agent (e.g., a pathogen) or a tumor, the method comprising administering to the subject (a) in combination with, to thereby prevent, treat, or ameliorate the infection in the subject.

Provided herein is a (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject that has received at least a first dose of a vaccine regimen against an infective agent (e.g., a pathogen) or a tumor, the method comprising administering (b) to the subject, to thereby prevent, treat, or ameliorate the infection the subject.

Provided herein is a combination composition described herein or combination therapy described herein for use in a method of preventing, treating, or ameliorating an infection a subject that has received at least a first dose of a vaccine regimen against an infective agent (e.g., a pathogen) or a tumor, the method comprising administering to the subject the combination composition described herein, or the combination therapy described herein, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an infection a subject that has received at least a first dose of a vaccine regimen against an infective agent (e.g., a pathogen) or a tumor, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

Provided herein is a combination composition described herein or combination therapy described herein for the manufacture of a medicament for use in a method of preventing, treating, or ameliorating an infection a subject that has received at least a first dose of a vaccine regimen against an infective agent (e.g., a pathogen) or a tumor.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

For the sake of clarity it is to be understood that the following embodiments are applicable to any of the foregoing aspects.

In some embodiments, the hIL-10R binding agent is administered after the immunogen, e.g., at least 24 hours to 12 months after the immunogen, e.g., at least 24 hours to 11 months, at least 24 hours to 10 months, at least 24 hours to 9 months, at least 24 hours to 8 months, at least 24 hours to 7 months, at least 24 hours to 6 months, at least 24 hours to 5 months, at least 24 hours to 4 months, at least 24 hours to 3 months, at least 24 hours to 2 months, at least 24 hours to 1 month, at least 24 hours to 3 weeks, at least 24 hours to 2 weeks, at least 24 hours to 1 week, at least 48 hours to 11 months, at least 48 hours to 10 months, at least 48 hours to 9 months, at least 48 hours to 8 months, at least 48 hours to 7 months, at least 48 hours to 6 months, at least 48 hours to 5 months, at least 48 hours to 4 months, at least 48 hours to 3 months, at least 48 hours to 2 months, at least 48 hours to 1 month, at least 48 hours to 3 weeks, at least 48 hours to 2 weeks, at least 48 hours to 1 week, at least 1 week to 11 months, at least 1 week to 10 months, at least 1 week to 9 months, at least 1 week to 8 months, at least 1 week to 7 months, at least 1 week to 6 months, at least 1 week to 5 months, at least 1 week to 4 months, at least 1 week to 3 months, at least 1 week to 2 months, at least 1 week to 1 month, at least 1 week to 3 weeks, at least 1 week to 2 weeks, at least 2 weeks to 11 months, at least 2 weeks to 10 months, at least 2 weeks to 9 months, at least 2 weeks to 8 months, at least 2 weeks to 7 months, at least 2 weeks to 6 months, at least 2 weeks to 5 months, at least 2 weeks to 4 months, at least 2 weeks to 3 months, at least 2 weeks to 2 months, at least 2 weeks to 1 month, at least 2 weeks to 3 weeks, at least 3 weeks to 2 months, at least 3 weeks to 11 months, at least 3 weeks to 10 months, at least 3 weeks to 9 months, at least 3 weeks to 8 months, at least 3 weeks to 7 months, at least 3 weeks to 6 months, at least 3 weeks to 5 months, at least 3 weeks to 4 months, at least 3 weeks to 3 months, at least 3 weeks to 2 months, or at least 3 weeks to 1 month after the immunogen.

In some embodiments, the hIL-10R binding agent is administered after the immunogen, e.g., from about 24 hours and 3 months after the immunogen, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the immunogen.

In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the immunogen.

In some embodiments, before the administering, the subject tested negative for the infection. In some embodiments, the infection is acute.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.2.2 Methods of Ameliorating, Treating, or Preventing an Infection in Vulnerable Sub-Populations of Subjects

In one aspect, provided herein are methods of ameliorating, treating, or preventing an infection (e.g., a viral infection, e.g., a SARS-CoV-2 infection) in a subject that is characterized as being a part of a sub-population of subjects vulnerable to infection or to severe disease associated with the infection, the method comprising administering to the subject (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby ameliorate, treat, or prevent the infection in the subject. In some embodiments, the hIL-10R binding agent (i.e., (b)(i)-(vi) is administered to the subject in an amount and for a time sufficient to prevent, ameliorate, or treat the infection in the subject. In some embodiments, prior to the administering, the subject has tested negative for the infection.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In one aspect, provided herein are methods of preventing, treating, or ameliorating an infection (e.g., of an infective agent) in a subject that is characterized as being a part of a sub-population of subjects vulnerable to infection or to severe disease associated with the infection, the method comprising administering to a subject (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); in combination with (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (a) and/or (b) are administered to the subject in an amount and for a time sufficient to prevent, treat, or ameliorate the infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection in a subject that is characterized as being a part of a sub-population of subjects vulnerable to infection or to severe disease associated with the infection, the method comprising administering to the subject (b) in combination with (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), to thereby prevent, treat, or ameliorate the infection in the subject.

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject that is characterized as being a part of a sub-population of subjects vulnerable to infection or to severe disease associated with the infection, the method comprising administering (a) and (b) to the subject, to thereby prevent, treat, or ameliorate the infection the subject.

Provided herein is a combination composition described herein or combination therapy described herein for use in a method of preventing, treating, or ameliorating an infection a subject that is characterized as being a part of a sub-population of subjects vulnerable to infection or to severe disease associated with the infection, the method comprising administering to the subject the combination composition described herein, or the combination therapy described herein, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of preventing, treating, or ameliorating an infection a subject that is characterized as being a part of a sub-population of subjects vulnerable to infection or to severe disease associated with the infection, the method comprising administering (a) in combination with (b) to the subject, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an infection a subject that is characterized as being a part of a sub-population of subjects vulnerable to infection or to severe disease associated with the infection, wherein the medicament is administered to the subject in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

Provided herein is a combination composition described herein or combination therapy described herein for the manufacture of a medicament for use in a method of preventing, treating, or ameliorating an infection a subject that is characterized as being a part of a sub-population of subjects vulnerable to infection or to severe disease associated with the infection.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In one aspect, provided herein are methods of preventing, treating, or ameliorating an infection (e.g., of an infective agent) in a subject that is characterized as being a part of a sub-population of subjects vulnerable to infection or to severe disease associated with the infection, the method comprising (a) first administering to a subject at least a first dose of an (a) immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), and (b) thereafter (e.g., as a booster) administering to the subject a hIL-10R binding agent e.g., comprising (i) a (b) hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (a) and/or (b) are administered to the subject in an amount and for a time sufficient to prevent, treat, or ameliorate the infection in the subject that is characterized as being a part of a sub-population of subjects vulnerable to infection or to severe disease associated with the infection.

Provided herein is (b) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, treating, or ameliorating an infection in a subject that is characterized as being a part of a sub-population of subjects vulnerable to infection or to severe disease associated with the infection, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a combination of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject that is characterized as being a part of a sub-population of subjects vulnerable to infection or to severe disease associated with the infection, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a use of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) for the manufacture of a medicament for use in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject that is characterized as being a part of a sub-population of subjects vulnerable to infection or to severe disease associated with the infection, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in a method of preventing, treating, or ameliorating an infection a subject that is characterized as being a part of a sub-population of subjects vulnerable to infection or to severe disease associated with the infection, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), wherein the method comprises (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the method further comprises administering the immunogen (e.g., (a)(i)-(vi)) to the subject in combination with the administration of the hIL-10R binding agent (e.g., (b)(i)-(vi)). In some embodiments, hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)).

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject. In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In one aspect, provided herein are methods of preventing, treating, or ameliorating an infection (e.g., of an infective agent) in a subject that is characterized as being a part of a sub-population of subjects vulnerable to infection or to severe disease associated with the infection, the method comprising administering to a subject that has received at least a first dose of a vaccine regimen against an infective agent (e.g., a pathogen) or a tumor (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the infection in the subject. In some embodiments, (b) is administered to the subject in an amount and for a time sufficient to prevent, treat, or ameliorate the infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection in a subject that is characterized as being a part of a sub-population of subjects vulnerable to infection or to severe disease associated with the infection that has received at least a first dose of a vaccine regimen against an infective agent (e.g., a pathogen) or a tumor, the method comprising administering to the subject (a) in combination with, to thereby prevent, treat, or ameliorate the infection in the subject.

Provided herein is a (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an infection a subject that is characterized as being a part of a sub-population of subjects vulnerable to infection or to severe disease associated with the infection that has received at least a first dose of a vaccine regimen against an infective agent (e.g., a pathogen) or a tumor, the method comprising administering (b) to the subject, to thereby prevent, treat, or ameliorate the infection the subject.

Provided herein is a combination composition described herein or combination therapy described herein for use in a method of preventing, treating, or ameliorating an infection a subject that is characterized as being a part of a sub-population of subjects vulnerable to infection or to severe disease associated with the infection that has received at least a first dose of a vaccine regimen against an infective agent (e.g., a pathogen) or a tumor, the method comprising administering to the subject the combination composition described herein, or the combination therapy described herein, to thereby prevent, treat, or ameliorate the infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an infection a subject that is characterized as being a part of a sub-population of subjects vulnerable to infection or to severe disease associated with the infection that has received at least a first dose of a vaccine regimen against an infective agent (e.g., a pathogen) or a tumor, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

Provided herein is a combination composition described herein or combination therapy described herein for the manufacture of a medicament for use in a method of preventing, treating, or ameliorating an infection a subject that is characterized as being a part of a sub-population of subjects vulnerable to infection or to severe disease associated with the infection that has received at least a first dose of a vaccine regimen against an infective agent (e.g., a pathogen) or a tumor.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

For the sake of clarity it is to be understood that the following embodiments are applicable to any of the foregoing aspects.

In some embodiments, the hIL-10R binding agent is administered after the immunogen, e.g., at least 24 hours to 12 months after the immunogen, e.g., at least 24 hours to 11 months, at least 24 hours to 10 months, at least 24 hours to 9 months, at least 24 hours to 8 months, at least 24 hours to 7 months, at least 24 hours to 6 months, at least 24 hours to 5 months, at least 24 hours to 4 months, at least 24 hours to 3 months, at least 24 hours to 2 months, at least 24 hours to 1 month, at least 24 hours to 3 weeks, at least 24 hours to 2 weeks, at least 24 hours to 1 week, at least 48 hours to 11 months, at least 48 hours to 10 months, at least 48 hours to 9 months, at least 48 hours to 8 months, at least 48 hours to 7 months, at least 48 hours to 6 months, at least 48 hours to 5 months, at least 48 hours to 4 months, at least 48 hours to 3 months, at least 48 hours to 2 months, at least 48 hours to 1 month, at least 48 hours to 3 weeks, at least 48 hours to 2 weeks, at least 48 hours to 1 week, at least 1 week to 11 months, at least 1 week to 10 months, at least 1 week to 9 months, at least 1 week to 8 months, at least 1 week to 7 months, at least 1 week to 6 months, at least 1 week to 5 months, at least 1 week to 4 months, at least 1 week to 3 months, at least 1 week to 2 months, at least 1 week to 1 month, at least 1 week to 3 weeks, at least 1 week to 2 weeks, at least 2 weeks to 11 months, at least 2 weeks to 10 months, at least 2 weeks to 9 months, at least 2 weeks to 8 months, at least 2 weeks to 7 months, at least 2 weeks to 6 months, at least 2 weeks to 5 months, at least 2 weeks to 4 months, at least 2 weeks to 3 months, at least 2 weeks to 2 months, at least 2 weeks to 1 month, at least 2 weeks to 3 weeks, at least 3 weeks to 2 months, at least 3 weeks to 11 months, at least 3 weeks to 10 months, at least 3 weeks to 9 months, at least 3 weeks to 8 months, at least 3 weeks to 7 months, at least 3 weeks to 6 months, at least 3 weeks to 5 months, at least 3 weeks to 4 months, at least 3 weeks to 3 months, at least 3 weeks to 2 months, or at least 3 weeks to 1 month after the immunogen.

In some embodiments, the hIL-10R binding agent is administered after the immunogen, e.g., from about 24 hours and 3 months after the immunogen, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the immunogen.

In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the immunogen.

In some embodiments, before the administering, the subject tested negative for the infection. In some embodiments, the infection is acute.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.2.3 Methods of Ameliorating, Treating, or Preventing an Acute Infection

In one aspect, provided herein are methods of preventing, treating, or ameliorating an acute infection (e.g., of an infective agent) in a subject, the method comprising administering to a subject (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject. In some embodiments, (b) is administered to the subject in an amount and for a time sufficient to prevent, treat, or ameliorate the acute infection in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of preventing, treating, or ameliorating an acute infection (e.g., of an infective agent) in a subject, the method comprising administering to a subject (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); in combination with (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject. In some embodiments, (a) and/or (b) are administered to the subject in an amount and for a time sufficient to prevent, treat, or ameliorate the acute infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection in a subject, the method comprising administering to the subject (b) in combination with (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), to thereby prevent, treat, or ameliorate the acute infection in the subject.

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection a subject, the method comprising administering (a) and (b) to the subject, to thereby prevent, treat, or ameliorate the acute infection the subject.

Provided herein is a combination composition described herein or combination therapy described herein for use in a method of preventing, treating, or ameliorating an acute infection a subject, the method comprising administering to the subject the combination composition described herein, or the combination therapy described herein, to thereby prevent, treat, or ameliorate the acute infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of preventing, treating, or ameliorating an acute infection a subject, the method comprising administering (a) in combination with (b) to the subject, to thereby prevent, treat, or ameliorate the acute infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an acute infection a subject in need thereof, wherein the medicament is administered to the subject in combination with (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

Provided herein is a combination composition described herein or combination therapy described herein for the manufacture of a medicament for use in a method of preventing, treating, or ameliorating an acute infection a subject.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In some embodiments, before the administering, the subject tested positive for the infection. In some embodiments, the hIL-10R binding agent (e.g., (a)(i)-(vi)) is administered within from about 1-10 days, 1-9 days, 1-8 days, 1-7 days, 1-6 days, 1-5 days, 1-4 days, 1-3 days, 1-2 days of the onset of one or more symptom of the infection or a positive test for the infection. In some embodiments, the hIL-10R binding agent (e.g., (a)(i)-(vi)) is administered within about 1, 2, 3, 4, 5, 6, 7, 8, 9, or 10 days from the onset of one or more symptom of the infection or a positive test for the infection.

In one aspect, provided herein are methods of preventing, treating, or ameliorating an acute infection (e.g., of an infective agent) in a subject, the method comprising (a) first administering to a subject at least a first dose of an (a) immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), and (b) thereafter (e.g., as a booster) administering to the subject a hIL-10R binding agent e.g., comprising (i) a (b) hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject. In some embodiments, (a) and/or (b) are administered to the subject in an amount and for a time sufficient to prevent, treat, or ameliorate the acute infection in the subject.

Provided herein is (b) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, treating, or ameliorating an acute infection in a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject.

Provided herein is a combination of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject.

Provided herein is a use of (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) for the manufacture of a medicament for use in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection a subject, the method comprising (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in a method of preventing, treating, or ameliorating an acute infection a subject in need thereof, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), wherein the method comprises (a) first administering to a subject at least a first dose of an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) and (b) thereafter (e.g., as a booster) administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the method further comprises administering the immunogen (e.g., (a)(i)-(vi)) to the subject in combination with the administration of the hIL-10R binding agent (e.g., (b)(i)-(vi)). In some embodiments, hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)).

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject. In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In some embodiments, before the administering, the subject tested positive for the infection. In some embodiments, the hIL-10R binding agent (e.g., (a)(i)-(vi)) is administered within from about 1-10 days, 1-9 days, 1-8 days, 1-7 days, 1-6 days, 1-5 days, 1-4 days, 1-3 days, 1-2 days of the onset of one or more symptom of the infection or a positive test for the infection. In some embodiments, the hIL-10R binding agent (e.g., (a)(i)-(vi)) is administered within about 1, 2, 3, 4, 5, 6, 7, 8, 9, or 10 days from the onset of one or more symptom of the infection or a positive test for the infection.

In one aspect, provided herein are methods of treating a human subject exposed to an infective agent, comprising administering to the subject (a) an immunogen from the infective agent comprising, e.g., (i) an immunogenic protein (e.g., described herein) (or an immunogenic fragment or variant thereof) (or a fusion protein or conjugate thereof), (ii) a nucleic acid (e.g., RNA) molecule comprising a coding region encoding an immunogenic protein (e.g., described herein) (or an immunogenic fragment or variant thereof) (or a fusion protein or conjugate thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), in combination with (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

Provided herein is (a) an immunogen (e.g., from the infective agent) e.g., comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v) for use in a method of treating a human subject exposed to an infective agent, the method comprising administering to the subject (a) in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby treat a human subject exposed to an infective agent.

Provided herein is a combination of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of treating a human subject exposed to an infective agent, the method comprising administering (a) and (b) to the subject, to thereby treat a human subject exposed to an infective agent.

Provided herein is a combination composition described herein or combination therapy described herein for use in a method of treating a human subject exposed to an infective agent, the method comprising administering to the subject the combination composition described herein, or the combination therapy described herein, to treat a human subject exposed to an infective agent.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of treating a human subject exposed to an infective agent, the method comprising administering (a) and (b) to the subject, to thereby treat a human subject exposed to an infective agent.

Provided herein is a use of (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for the manufacture of a medicament for use in a method of treating a human subject exposed to an infective agent, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby treat a human subject exposed to an infective agent.

Provided herein is a combination composition described herein or combination therapy described herein for the manufacture of a medicament for use in a method of treating a human subject exposed to an infective agent.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the infective agent is a virus. In some embodiments, the infective agent is a coronavirus (e.g., a SARS-CoV-2 virus, a SARS-CoV virus, or a MERS-CoV SARS-CoV-2 virus). In some embodiments, the infective agent is a SARS-CoV-2 virus.

In some embodiments, the subject has an acute infection with the infective agent. In some embodiments, the subject has a post viral syndrome (e.g., long Covid) from a previous acute viral infection.

In some embodiments, the immunogen is the same as a vaccine immunogen. In some embodiments, the subject has not previously had at least one vaccine dose against the infective agent. In some embodiments, the subject has previously had at least one vaccine dose against the infective agent.

In some embodiments, before the administering, the subject tested positive for the infection. In some embodiments, the hIL-10R binding agent (e.g., (a)(i)-(vi)) is administered within from about 1-10 days, 1-9 days, 1-8 days, 1-7 days, 1-6 days, 1-5 days, 1-4 days, 1-3 days, 1-2 days of the onset of one or more symptom of the infection or a positive test for the infection. In some embodiments, the hIL-10R binding agent (e.g., (a)(i)-(vi)) is administered within about 1, 2, 3, 4, 5, 6, 7, 8, 9, or 10 days from the onset of one or more symptom of the infection or a positive test for the infection.

In one aspect, provided herein are methods of preventing, treating, or ameliorating an acute infection (e.g., of an infective agent) in a subject, the method comprising administering to a subject that has received at least a first dose of a vaccine regimen against an infective agent (e.g., a pathogen) or a tumor (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, treat, or ameliorate the acute infection in the subject. In some embodiments, (b) is administered to the subject in an amount and for a time sufficient to prevent, treat, or ameliorate the acute infection in the subject.

Provided herein is (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection in a subject that has received at least a first dose of a vaccine regimen against an infective agent (e.g., a pathogen) or a tumor, the method comprising administering to the subject (a) in combination with, to thereby prevent, treat, or ameliorate the acute infection in the subject.

Provided herein is a (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, treating, or ameliorating an acute infection a subject that has received at least a first dose of a vaccine regimen against an infective agent (e.g., a pathogen) or a tumor, the method comprising administering (b) to the subject, to thereby prevent, treat, or ameliorate the acute infection the subject.

Provided herein is a combination composition described herein or combination therapy described herein for use in a method of preventing, treating, or ameliorating an acute infection a subject that has received at least a first dose of a vaccine regimen against an infective agent (e.g., a pathogen) or a tumor, the method comprising administering to the subject the combination composition described herein, or the combination therapy described herein, to thereby prevent, treat, or ameliorate the acute infection the subject.

Provided herein is a use of (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for the manufacture of a medicament for use in a method of preventing, treating, or ameliorating an acute infection a subject that has received at least a first dose of a vaccine regimen against an infective agent (e.g., a pathogen) or a tumor, wherein the medicament is administered to the subject in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

Provided herein is a combination composition described herein or combination therapy described herein for the manufacture of a medicament for use in a method of preventing, treating, or ameliorating an acute infection a subject that has received at least a first dose of a vaccine regimen against an infective agent (e.g., a pathogen) or a tumor.

In some embodiments, the (a) immunogen (e.g., from the infective agent) comprises (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) the hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

For the sake of clarity it is to be understood that the following embodiments are applicable to any of the foregoing aspects.

In some embodiments, before the administering, the subject tested positive for the infection. In some embodiments, the hIL-10R binding agent (e.g., (a)(i)-(vi)) is administered within from about 1-10 days, 1-9 days, 1-8 days, 1-7 days, 1-6 days, 1-5 days, 1-4 days, 1-3 days, 1-2 days of the onset of one or more symptom of the infection or a positive test for the infection. In some embodiments, the hIL-10R binding agent (e.g., (a)(i)-(vi)) is administered within about 1, 2, 3, 4, 5, 6, 7, 8, 9, or 10 days from the onset of one or more symptom of the infection or a positive test for the infection.

In some embodiments, the hIL-10R binding agent is administered after the immunogen, e.g., at least 24 hours to 12 months after the immunogen, e.g., at least 24 hours to 11 months, at least 24 hours to 10 months, at least 24 hours to 9 months, at least 24 hours to 8 months, at least 24 hours to 7 months, at least 24 hours to 6 months, at least 24 hours to 5 months, at least 24 hours to 4 months, at least 24 hours to 3 months, at least 24 hours to 2 months, at least 24 hours to 1 month, at least 24 hours to 3 weeks, at least 24 hours to 2 weeks, at least 24 hours to 1 week, at least 48 hours to 11 months, at least 48 hours to 10 months, at least 48 hours to 9 months, at least 48 hours to 8 months, at least 48 hours to 7 months, at least 48 hours to 6 months, at least 48 hours to 5 months, at least 48 hours to 4 months, at least 48 hours to 3 months, at least 48 hours to 2 months, at least 48 hours to 1 month, at least 48 hours to 3 weeks, at least 48 hours to 2 weeks, at least 48 hours to 1 week, at least 1 week to 11 months, at least 1 week to 10 months, at least 1 week to 9 months, at least 1 week to 8 months, at least 1 week to 7 months, at least 1 week to 6 months, at least 1 week to 5 months, at least 1 week to 4 months, at least 1 week to 3 months, at least 1 week to 2 months, at least 1 week to 1 month, at least 1 week to 3 weeks, at least 1 week to 2 weeks, at least 2 weeks to 11 months, at least 2 weeks to 10 months, at least 2 weeks to 9 months, at least 2 weeks to 8 months, at least 2 weeks to 7 months, at least 2 weeks to 6 months, at least 2 weeks to 5 months, at least 2 weeks to 4 months, at least 2 weeks to 3 months, at least 2 weeks to 2 months, at least 2 weeks to 1 month, at least 2 weeks to 3 weeks, at least 3 weeks to 2 months, at least 3 weeks to 11 months, at least 3 weeks to 10 months, at least 3 weeks to 9 months, at least 3 weeks to 8 months, at least 3 weeks to 7 months, at least 3 weeks to 6 months, at least 3 weeks to 5 months, at least 3 weeks to 4 months, at least 3 weeks to 3 months, at least 3 weeks to 2 months, or at least 3 weeks to 1 month after the immunogen.

In some embodiments, the hIL-10R binding agent is administered after the immunogen, e.g., from about 24 hours and 3 months after the immunogen, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the immunogen.

In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the immunogen. In some embodiments, the hIL-10R binding agent is administered after the immunogen, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the immunogen.

In some embodiments, before the administering, the subject tested negative for the acute infection. In some embodiments, the acute infection is acute.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the acute infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.3 Methods of Ameliorating, Treating, or Preventing Infection Associated Disease

In one aspect, provided herein are methods of preventing, ameliorating, or treating disease associated with an infection (e.g., a viral infection, e.g., a SARS-CoV-2 infection) in a subject, the method comprising administering to the subject (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (a)(i)-(iv), or (vi) a pharmaceutical composition comprising any one of (b)(i)-(v), to thereby prevent, ameliorate, or treat the disease associated with the infection in the subject. In some embodiments, the hIL-10R binding agent (i.e., (b)(i)-(vi) is administered to the subject in an amount and for a time sufficient to prevent, ameliorate, or treat disease associated with infection in the subject. In some embodiments, prior to the administering, the subject has tested negative for the infection.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of preventing, ameliorating, or treating disease associated with an infection (e.g., a viral infection, e.g., a SARS-CoV-2 infection) in a subject, the method comprising administering to the subject a vaccine against the infection, in combination with (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat the disease associated with the infection in the subject. In some embodiments, the hIL-10R binding agent (i.e., (b)(i)-(vi) is administered to the subject in an amount and for a time sufficient to prevent, ameliorate, or treat disease associated with infection in the subject. In some embodiments, prior to the administering, the subject has tested negative for the infection.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or treating disease associated with an infection (e.g., a viral infection, e.g., a SARS-CoV-2 infection) in a subject, the method comprising administering to the subject (b) in combination with a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby prevent, ameliorate, or treat disease associated with an infection (e.g., a viral infection, e.g., a SARS-CoV-2 infection) in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or treating disease associated with an infection (e.g., a viral infection, e.g., a SARS-CoV-2 infection) in a subject, the method comprising administering (a) in combination with (b) to the subject, to thereby prevent, ameliorate, or treat disease associated with an infection (e.g., a viral infection, e.g., a SARS-CoV-2 infection) in a subject.

Provided herein is a combination composition described herein or combination therapy described herein for use in a method of preventing, ameliorating, or treating disease associated with an infection (e.g., a viral infection, e.g., a SARS-CoV-2 infection) in a subject, the method comprising administering to the subject the combination composition described herein, or the combination therapy described herein, to thereby prevent, ameliorate, or treat disease associated with an infection (e.g., a viral infection, e.g., a SARS-CoV-2 infection) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), for use in a method of preventing, ameliorating, or treating disease associated with an infection (e.g., a viral infection, e.g., a SARS-CoV-2 infection) in a subject, the method comprising administering (a) in combination with (b) to the subject, to thereby prevent, ameliorate, or treat disease associated with an infection (e.g., a viral infection, e.g., a SARS-CoV-2 infection) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or treating disease associated with an infection (e.g., a viral infection, e.g., a SARS-CoV-2 infection) in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby prevent, ameliorate, or treat disease associated with an infection (e.g., a viral infection, e.g., a SARS-CoV-2 infection) in a subject.

Provided herein is a combination composition described herein or combination therapy described herein for the manufacture of a medicament for use in a method of preventing, ameliorating, or treating disease associated with an infection (e.g., a viral infection, e.g., a SARS-CoV-2 infection) in a subject.

In one aspect, provided herein are methods of preventing, ameliorating, or treating disease associated with an infection (e.g., a viral infection, e.g., a SARS-CoV-2 infection) in a subject vaccinated with (a) at least a first dose of an immunogen (e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (b) a hIL-10R binding agent comprising, e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat disease associated with an infection (e.g., a viral infection, e.g., a SARS-CoV-2 infection) in the subject. In some embodiments, (b) is administered to the subject in an amount and for a time sufficient to enhance the immunogen specific immune response in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or treating disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat disease associated with an infection (e.g., a viral infection, e.g., a SARS-CoV-2 infection) in a subject vaccinated with (a) at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or treating disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (a) in combination with (b), to thereby prevent, ameliorate, or treat disease associated with an infection (e.g., a viral infection, e.g., a SARS-CoV-2 infection) in a subject vaccinated with (a) at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method preventing, ameliorating, or treating disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

For the sake of clarity it is to be understood that the following embodiments are applicable to any of the foregoing aspects.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In some embodiments, the hIL-10R binding agent (e.g., (a)(i)-(vi)) is administered intramuscularly, subcutaneously, or mucosally (e.g., intranasally). In some embodiments, the hIL-10R binding agent (e.g., (a)(i)-(vi)) is administered mucosally (e.g., intranasally). In some embodiments, the hIL-10R binding agent (e.g., (b)(i)-(vi)) is administered intranasally.

In some embodiments, the subject has been vaccinated (e.g., partially vaccinated or fully vaccinated) against the infection with at least a first dose of (b) an immunogen comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v). In some embodiments, the at least a first dose of the immunogen (e.g., (b)(i)-(vi)) was administered intramuscularly, subcutaneously, or mucosally (e.g., intranasally). In some embodiments, the at least a first dose of the immunogen (e.g., (b)(i)-(vi)) was administered intramuscularly or subcutaneously. In some embodiments, the hIL-10R binding agent (e.g., (a)(i)-(vi)) was administered intranasally and the at least a first dose of the immunogen (e.g., (b)(i)-(vi)) is administered intramuscularly or subcutaneously.

In some embodiments, the hIL-10R binding agent (e.g., (a)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen was administered (e.g., (b)(i)-(vi)) to the subject. In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject.

In some embodiments, the method further comprises administering an immunogen (e.g., (b)(i)-(vi)) to the subject in combination with the administration of the hIL-10R binding agent (e.g., (a)(i)-(vi)). In some embodiments, the hIL-10R binding agent (e.g., (a)(i)-(vi)) is administered as a booster in a prime-boost regimen, wherein the prime portion of the regimen comprises administration of the at least a first dose of the immunogen to the subject (e.g., (b)(i)-(vi)); and the boost portion of the regimen comprises the administration of the immunogen (e.g., (b)(i)-(vi)) and the hIL-10R binding agent (e.g., (a)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the infection is a viral infection. In some embodiments, the infection is an acute infection. In some embodiments, the infection is a coronavirus infection (e.g., a SARS-CoV-2 virus infection, a SARS-CoV virus infection, or a MERS-CoV SARS-CoV-2 virus infection). In some embodiments, the infection is a SARS-CoV-2 virus infection.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.3.1 Methods of Ameliorating, Treating, or Preventing Severe Disease Associated with an Infection

In one aspect, provided herein are methods of preventing, ameliorating, or treating severe disease associated with an infection (e.g., a viral infection, e.g., a SARS-CoV-2 infection) in a subject, the method comprising administering to the subject (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat the severe disease associated with the infection in the subject. In some embodiments, the hIL-10R binding agent (i.e., (b)(i)-(vi) is administered to the subject to prevent, ameliorate, or treat severe disease associated with infection in the subject. In some embodiments, prior to the administering, the subject has tested negative for the infection.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of preventing, ameliorating, or treating severe disease associated with an infection (e.g., a viral infection, e.g., a SARS-CoV-2 infection) in a subject, the method comprising administering to the subject a vaccine against the infection, in combination with (b) a hIL-10R binding agent comprising, e.g., (i) a hIL-10R binding protein (e.g., described herein) (or a functional fragment or variant thereof), (ii) a nucleic acid molecule comprising a coding region encoding a hIL-10R binding protein (e.g., described herein) (or a functional fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat the severe disease associated with the infection in the subject. In some embodiments, the hIL-10R binding agent (i.e., (b)(i)-(vi) is administered to the subject in an amount and for a time sufficient to prevent, ameliorate, or treat severe disease associated with infection in the subject. In some embodiments, prior to the administering, the subject has tested negative for the infection.

In one aspect, provided herein are methods of preventing, ameliorating, or treating severe disease associated with an infection (e.g., a viral infection, e.g., a SARS-CoV-2 infection) in a subject, the method comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby prevent, ameliorate, or treat severe disease associated with an infection (e.g., a viral infection, e.g., a SARS-CoV-2 infection) in a subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or treating severe disease associated with an infection (e.g., a viral infection, e.g., a SARS-CoV-2 infection) in a subject, the method comprising administering to the subject (b) in combination with a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby prevent, ameliorate, or treat severe disease associated with an infection (e.g., a viral infection, e.g., a SARS-CoV-2 infection) in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or treating severe disease associated with an infection (e.g., a viral infection, e.g., a SARS-CoV-2 infection) in a subject, the method comprising administering (a) in combination with (b) to the subject, to thereby prevent, ameliorate, or treat severe disease associated with an infection (e.g., a viral infection, e.g., a SARS-CoV-2 infection) in a subject.

Provided herein is a combination composition described herein or combination therapy described herein for use in a method of preventing, ameliorating, or treating severe disease associated with an infection (e.g., a viral infection, e.g., a SARS-CoV-2 infection) in a subject, the method comprising administering to the subject the combination composition described herein, or the combination therapy described herein, to thereby prevent, ameliorate, or treat severe disease associated with an infection (e.g., a viral infection, e.g., a SARS-CoV-2 infection) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), for use in a method of preventing, ameliorating, or treating severe disease associated with an infection (e.g., a viral infection, e.g., a SARS-CoV-2 infection) in a subject, the method comprising administering (a) in combination with (b) to the subject, to thereby prevent, ameliorate, or treat severe disease associated with an infection (e.g., a viral infection, e.g., a SARS-CoV-2 infection) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or treating severe disease associated with an infection (e.g., a viral infection, e.g., a SARS-CoV-2 infection) in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby prevent, ameliorate, or treat severe disease associated with an infection (e.g., a viral infection, e.g., a SARS-CoV-2 infection) in a subject.

Provided herein is a combination composition described herein or combination therapy described herein for the manufacture of a medicament for use in a method of preventing, ameliorating, or treating severe disease associated with an infection (e.g., a viral infection, e.g., a SARS-CoV-2 infection) in a subject.

In one aspect, provided herein are methods of preventing, ameliorating, or treating severe disease associated with an infection (e.g., a viral infection, e.g., a SARS-CoV-2 infection) in a subject vaccinated with (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), the method comprising administering to the subject (b) a hIL-10R binding agent comprising, e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat severe disease associated with an infection (e.g., a viral infection, e.g., a SARS-CoV-2 infection) in the subject. In some embodiments, (b) is administered to the subject in an amount and for a time sufficient to enhance the immunogen specific immune response in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or treating severe disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby prevent, ameliorate, or treat severe disease associated with an infection (e.g., a viral infection, e.g., a SARS-CoV-2 infection) in a subject vaccinated with (a) at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or treating severe disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (a) in combination with (b), to thereby prevent, ameliorate, or treat severe disease associated with an infection (e.g., a viral infection, e.g., a SARS-CoV-2 infection) in a subject vaccinated with (a) at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method preventing, ameliorating, or treating severe disease associated with an infection (e.g., a viral infection, e.g., a SARS-CoV-2 infection) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

For the sake of clarity it is to be understood that the following embodiments are applicable to any of the foregoing aspects.

In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

In some embodiments, the hIL-10R binding agent (e.g., (a)(i)-(vi)) is administered intramuscularly, subcutaneously, or mucosally (e.g., intranasally). In some embodiments, the hIL-10R binding agent (e.g., (a)(i)-(vi)) is administered mucosally (e.g., intranasally). In some embodiments, the hIL-10R binding agent (e.g., (b)(i)-(vi)) is administered intranasally.

In some embodiments, the subject has been vaccinated (e.g., partially vaccinated or fully vaccinated) against the infection with at least a first dose of (b) an immunogen comprising (i) an immunogenic protein (e.g., described herein) (or an immunogenic fragment or variant thereof), (ii) a nucleic acid molecule comprising a coding region encoding an immunogenic protein (e.g., described herein) (or an immunogenic fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v). In some embodiments, the at least a first dose of the immunogen (e.g., (b)(i)-(vi)) was administered intramuscularly, subcutaneously, or mucosally (e.g., intranasally). In some embodiments, the at least a first dose of the immunogen (e.g., (b)(i)-(vi)) was administered intramuscularly or subcutaneously. In some embodiments, the hIL-10R binding agent (e.g., (a)(i)-(vi)) was administered intranasally and the at least a first dose of the immunogen (e.g., (b)(i)-(vi)) is administered intramuscularly or subcutaneously.

In some embodiments, the hIL-10R binding agent (e.g., (a)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen was administered (e.g., (b)(i)-(vi)) to the subject. In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject.

In some embodiments, the method further comprises administering an immunogen (e.g., (b)(i)-(vi)) to the subject in combination with the administration of the hIL-10R binding agent (e.g., (a)(i)-(vi)). In some embodiments, the hIL-10R binding agent (e.g., (a)(i)-(vi)) is administered as a booster in a prime-boost regimen, wherein the prime portion of the regimen comprises administration of the at least a first dose of the immunogen to the subject (e.g., (b)(i)-(vi)); and the boost portion of the regimen comprises the administration of the immunogen (e.g., (b)(i)-(vi)) and the hIL-10R binding agent (e.g., (a)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the infection is a viral infection. In some embodiments, the infection is an acute infection. In some embodiments, the infection is a coronavirus infection (e.g., a SARS-CoV-2 virus infection, a SARS-CoV virus infection, or a MERS-CoV SARS-CoV-2 virus infection). In some embodiments, the infection is a SARS-CoV-2 virus infection.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.3.2 Methods of Ameliorating, Treating, or Preventing Post Viral Syndrome

In one aspect, provided herein are methods of treating, ameliorating, or preventing post viral syndrome, e.g., long COVID, in a subject in need thereof, the method comprising administering to the subject (b) a hIL-10R binding agent comprising, e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (a)(i)-(iv), or (vi) a pharmaceutical composition comprising any one of (b)(i)-(v), to thereby prevent or treat post viral syndrome, e.g., long COVID, in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of treating, ameliorating, or preventing post viral syndrome, e.g., long COVID, in a subject in need thereof, the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby treat, ameliorate, or prevent post viral syndrome, e.g., long COVID, in a subject in need thereof.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of treating, ameliorating, or preventing post viral syndrome, e.g., long COVID, in a subject in need thereof.

In some embodiments, the post viral syndrome is long COVID. Long COVID is commonly used to describe signs and symptoms that continue or develop after acute COVID-19. It includes both ongoing symptomatic COVID-19 and post-COVID-19 syndrome. It usually presents with clusters of symptoms, often overlapping, which can fluctuate and change over time and can affect any system in the body. One group of common symptoms are mainly respiratory, such as a cough and feeling breathless (dyspnea) but also include fatigue and headaches. A second group of symptoms affects many parts of the body, including the heart, brain, and the gut. For example, heart symptoms such as palpitations or increased heartbeat, as well as pins and needles, numbness and ‘brain fog’ have been commonly reported.

In some embodiments, the long COVID results from infection with a SARS-CoV-2 virus, a SARS-CoV virus, or a MERS-CoV virus.

In some embodiments, the subject has tested positive for the infection prior to the administering of the hIL-10R binding agent (e.g., (a)(i)-(vi)). In some embodiments, the subject had tested positive for the infection; but tested negative for the infection prior to the administering of the hIL-10R binding agent (e.g., (a)(i)-(vi)). In some embodiments, the subject has been diagnosed with long COVID. In some embodiments, the subject has been diagnosed with one or more sign or symptom of long COVID.

In some embodiments, the subject has been vaccinated (e.g., partially vaccinated or fully vaccinated) against the infection with (b) at least a first dose of an immunogen e.g., comprising, e.g., (b)(i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(iv), prior to the administration of the hIL-10R binding agent (a).

In some embodiments, the hIL-10R binding agent (e.g., (a)(i)-(vi)) is administered intramuscularly, subcutaneously, or mucosally (e.g., intranasally). In some embodiments, the hIL-10R binding agent (e.g., (a)(i)-(vi)) is administered mucosally (e.g., intranasally). In some embodiments, the hIL-10R binding agent (e.g., (a)(i)-(vi)) is administered intranasally. In some embodiments, the at least a first dose of the immunogen (e.g., (b)(i)-(vi)) was administered intramuscularly, subcutaneously, or mucosally (e.g., intranasally). In some embodiments, the at least a first dose of the immunogen (e.g., (b)(i)-(vi)) was administered intramuscularly or subcutaneously. In some embodiments, the hIL-10R binding agent (e.g., (a)(i)-(vi)) was administered intranasally and the at least a first dose of the immunogen (e.g., (b)(i)-(vi)) is administered intramuscularly or subcutaneously.

In some embodiments, the hIL-10R binding agent (e.g., (a)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after the at least a first dose of the immunogen (e.g., (b)(i)-(vi)) was administered to the subject.

In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen was administered (e.g., (b)(i)-(vi)) to the subject. In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after the at least a first dose of the immunogen (e.g., (b)(i)-(vi)) was administered to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after the at least a first dose of the immunogen (e.g., (b)(i)-(vi)) was administered to the subject.

In some embodiments, the infection is a viral infection. In some embodiments, the infection is an acute infection. In some embodiments, the infection is a coronavirus infection (e.g., a SARS-CoV-2 virus infection, a SARS-CoV virus infection, or a MERS-CoV virus infection). In some embodiments, the infection is a SARS-CoV-2 virus infection.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (b)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (a)(i)-(vi)).

### 5.21.4 Methods of Enhancing an Immunogen-Specific Immune Response

In one aspect, provided herein are vaccines and methods of enhancing an immunogen-specific immune response in a subject in need thereof, the method comprising administering to the subject (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby enhance the immunogen-specific immune response in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are vaccines and methods of enhancing an immunogen-specific immune response in a subject in need thereof, the method comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby enhance the immunogen-specific immune response in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of enhancing an immunogen-specific immune response in a subject, the method comprising administering to the subject (b) in combination with a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby enhance an immunogen-specific immune response in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of enhancing an immunogen-specific immune response in a subject, the method comprising administering (a) in combination with (b) to the subject, to thereby enhance an immunogen-specific immune response in a subject.

Provided herein is a combination composition described herein or combination therapy described herein for use in a method of enhancing an immunogen-specific immune response in a subject, the method comprising administering to the subject the combination composition described herein, or the combination therapy described herein, to thereby enhance an immunogen-specific immune response in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), for use in a method of enhancing an immunogen-specific immune response in a subject, the method comprising administering (a) in combination with (b) to the subject, to thereby enhance an immunogen-specific immune response in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of enhancing an immunogen-specific immune response in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby enhance an immunogen-specific immune response in a subject.

Provided herein is a combination composition described herein or combination therapy described herein for the manufacture of a medicament for use in a method of enhancing an immunogen-specific immune response in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are vaccines and methods of enhancing an immunogen-specific immune response in a subject vaccinated with (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), the method comprising administering to the subject (b) a hIL-10R binding agent comprising, e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby enhance the immunogen-specific immune response in the subject. In some embodiments, (b) is administered to the subject in an amount and for a time sufficient to enhance the immunogen specific immune response in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of enhancing an immunogen-specific immune response in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby enhance an immunogen-specific immune response in a subject vaccinated with (a) at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of enhancing an immunogen-specific immune response in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (a) in combination with (b), to thereby enhance an immunogen-specific immune response in a subject vaccinated with (a) at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of enhancing an immunogen-specific immune response in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

For the sake of clarity it is to be understood that the following embodiments are applicable to any of the foregoing aspects.

Increasing an immunogen-specific immune responses includes increasing the duration of an immune response, increasing the magnitude of an immune response, and/or changing the nature of the immune response (e.g., inducing immunogen specific IgA antibodies and/or immunogen specific IgG antibodies).

In some embodiments, the mucosal (e.g., nasal mucosa, respiratory tract (e.g., upper respiratory tract, lower respiratory tract), gastrointestinal mucosa, urogenital mucosa, oral mucosa, ear mucosa, etc.) immune response is enhanced.

In some embodiments, the level of immunogen specific IgA antibodies is increased by at least about 5%, 10%, 15%, 20%, 25%, 30%, 35%, 40%, 45%, 50%, 55%, 60%, 65%, 70%, 75%, 80%, 85%, 90%, or 95%, or more (e.g., relative to a control composition that does not contain the hIL-10R binding agent (e.g., a hIL-10R binding protein (or a functional fragment and/or functional variant thereof). In some embodiments, the level of immunogen specific IgA antibodies is increased from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

In some embodiments, the level of immunogen specific IgG antibodies is increased by at least about 5%, 10%, 15%, 20%, 25%, 30%, 35%, 40%, 45%, 50%, 55%, 60%, 65%, 70%, 75%, 80%, 85%, 90%, or 95%, or more (e.g., relative to a control composition that does not contain the hIL-10R binding agent (e.g., a hIL-10R binding protein (or a functional fragment and/or functional variant thereof). In some embodiments, the level of immunogen specific IgG antibodies is suppressed from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

In some embodiments, the method further comprises administering the immunogen (e.g., (a)(i)-(vi)) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the at least a first dose of the immunogen to the subject; and the boost portion of the regimen comprises the administration of the immunogen and the hIL-10R binding agent (e.g., (b)(i)-(vi)). In some embodiments, the boost portion of the regimen is administered intramuscularly, subcutaneously, or mucosally (e.g., intranasally, into the ear (or a specific compartment thereof (e.g., the middle ear, inner ear, etc.), orally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent (e.g., (b)(i)-(vi)) is administered intranasally and the at least a first dose of the immunogen (e.g., (a)(i)-(vi)) is administered intramuscularly or subcutaneously.

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

An immune response in a subject can be measured by common methods known to those of skill in the art. For example, titers of salivary immunogen specific IgA and/or IgG antibodies can be assess post administration. For example, nasal and/or salivary swabs can be taken from subjects and the level of immunogen specific IgA and/or IgG antibodies quantified using an enzyme-linked immunosorbent assay (ELISA). An ELISA is a standard laboratory test for detecting and quantifying antibodies well known to the person of skill in the art. Generally, the samples (e.g., nasal swabs, saliva, blood, etc.) are processed according to standard techniques. The recombinant target antigen (e.g., the immunogen) is immobilized in microplate wells. The microplate is blocked by through the incubation with an irrelevant antigen (e.g., bovine serum albumin). The sample from the subject is prepared and added to the blocked wells to allow for binding of the immunogen specific antibodies to the immobilized immunogen. The bound antibodies can be detected using a secondary tagged antibody that binds to the previously bound antibodies (e.g., anti-human IgA antibodies, anti-human IgG antibodies). See, e.g., Havervall S. et al., Anti-Spike Mucosal IgA Protection against SARS-CoV-2 Omicron Infection, N Engl J Med 2022; 387:1333-1336, DOI: 10.1056/NEJMc2209651; Sano, K., Bhavsar, D., Singh, G. et al. SARS-CoV-2 vaccination induces mucosal antibody responses in previously infected individuals. Nat Commun 13, 5135 (2022). https://doi.org/10.1038/s41467-022-32389-8; Yannick G. et al. Humoral Responses and Serological Assays in SARS-CoV-2 Infections, Frontiers in Immunology, Vol 11 (2020) 10.3389/fimmu.2020.610688; Forgacs David et al., SARS-CoV-2 mRNA Vaccines Elicit Different Responses in Immunologically Naïve and Pre-Immune Humans; Front. Immunol., Vol 12 (27 Sep. 2021) https://doi.org/10.3389/fimmu.2021.728021, the entire contents of each of which is incorporated by reference herein for all purposes.

Cell based assays can also be utilized to detect a cell based immune response (e.g., T cell immune response). For example, immunogen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4+ T Cell Responses Not Measured by Assays Conventionally Used in Clinical Trials.” Vaccines vol. 6,3 50. 31 Jul. 2018, doi:10.3390/vaccines6030050, the entire contents of which is incorporated by reference herein for all purposes.

### 5.21.5 Methods of Increasing the Level of Immunogen-Specific Mucosal IgA

In one aspect, provided herein are methods of increasing the level of immunogen-specific mucosal IgA in a subject in need thereof, the method comprising administering to the subject (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby increase the level of immunogen-specific mucosal IgA in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of increasing the level of immunogen-specific mucosal IgA in a subject in need thereof, the method comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby increase the level of immunogen-specific mucosal IgA in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of increasing the level of immunogen-specific mucosal IgA in a subject, the method comprising administering to the subject (b) in combination with a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby increase the level of immunogen-specific mucosal IgA in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of increasing the level of immunogen-specific mucosal IgA in a subject, the method comprising administering (a) in combination with (b) to the subject, to thereby increase the level of immunogen-specific mucosal IgA in a subject.

Provided herein is a combination composition described herein or combination therapy described herein for use in a method of increasing the level of immunogen-specific mucosal IgA in a subject, the method comprising administering to the subject the combination composition described herein, or the combination therapy described herein, to thereby increase the level of immunogen-specific mucosal IgA in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), for use in a method of increasing the level of immunogen-specific mucosal IgA in a subject, the method comprising administering (a) in combination with (b) to the subject, to thereby increase the level of immunogen-specific mucosal IgA in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of increasing the level of immunogen-specific mucosal IgA in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby increase the level of immunogen-specific mucosal IgA in a subject.

Provided herein is a combination composition described herein or combination therapy described herein for the manufacture of a medicament for use in a method of increasing the level of immunogen-specific mucosal IgA in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are methods of increasing the level of immunogen-specific mucosal IgA in a subject vaccinated with (a) at least a first dose of an immunogen e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), the method comprising administering to the subject (b) a hIL-10R binding protein e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby increase the level of immunogen-specific mucosal IgA in the subject. In some embodiments, (b) is administered to the subject in an amount and for a time sufficient to increase the level of immunogen specific mucosal IgA in the subject.

In one aspect, provided herein are methods of increasing the level of immunogen-specific IgG (e.g., mucosal IgG, circulating IgG) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (b) a hIL-10R binding protein e.g., comprising, (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby increase the level of immunogen-specific IgG in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of increasing the level of immunogen-specific mucosal IgA in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby increasing the level of immunogen-specific mucosal IgA in a subject vaccinated with (a) at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of increasing the level of immunogen-specific mucosal IgA in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (a) in combination with (b), to thereby increase the level of immunogen-specific mucosal IgA in a subject vaccinated with (a) at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of increasing the level of immunogen-specific mucosal IgA in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

For the sake of clarity it is to be understood that the following embodiments are applicable to any of the foregoing aspects.

In some embodiments, the level of immunogen specific mucosal IgA is increased by at least about 1-fold, 10-fold, 100-fold, 1,000-fold, or 10,000-fold.

In some embodiments, the level of immunogen specific mucosal IgA antibodies is increased by at least about 5%, 10%, 15%, 20%, 25%, 30%, 35%, 40%, 45%, 50%, 55%, 60%, 65%, 70%, 75%, 80%, 85%, 90%, or 95%, or more (e.g., relative to a control composition that does not contain the hIL-10R binding agent (e.g., a hIL-10R binding protein (or a functional fragment and/or functional variant thereof). In some embodiments, the level of immunogen specific mucosal IgA antibodies is increased from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

In some embodiments, the method further comprises administering the immunogen (e.g., (a)(i)-(vi)) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent (e.g., (b)(i)-(vi)) is administered intranasally and the at least a first dose of the immunogen (e.g., (a)(i)-(vi)) is administered intramuscularly or subcutaneously.

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

Mucosa includes, e.g., the mucosa of the respiratory tract (e.g., the upper respiratory tract (e.g., nasal mucosa), and/or the lower respiratory tract (e.g., the lungs)), mucosa of the gastrointestinal tract, urogenital mucosa, oral mucosa, ear mucosa, etc. In some embodiments, the level of respiratory tract (e.g., the upper respiratory tract (e.g., nasal mucosa), and/or the lower respiratory tract (e.g., the lungs)) mucosal IgA is increased. In some embodiments, the level of upper respiratory tract mucosal IgA is increased. In some embodiments, the level of lower respiratory tract (e.g., the lung)) mucosal IgA is increased. In some embodiments, the level of nasal mucosal IgA is increased.

Mucosal IgA can be measured by common methods known to those of skill in the art. For example, titers of salivary or nasal immunogen specific IgA antibodies can be assessed post administration. For example, nasal or salivary swabs can be taken from subjects and the level of immunogen specific IgA antibodies quantified using an ELISA. As described above, an ELISA is a standard laboratory test for detecting and quantifying antibodies well known to the person of skill in the art. Generally, the samples (e.g., nasal swabs, saliva, etc.) are processed according to standard techniques. The recombinant target antigen (e.g., the immunogen) is immobilized in microplate wells. The microplate is blocked by through the incubation with an irrelevant antigen (e.g., bovine serum albumin). The sample from the subject is prepared and added to the blocked wells to allow for binding of the immunogen specific antibodies to the immobilized immunogen. The bound antibodies can be detected using a secondary tagged antibody that binds to the previously bound antibodies (e.g., anti-human IgA antibodies, anti-human IgG antibodies). See, e.g., Havervall S. et al., Anti-Spike Mucosal IgA Protection against SARS-CoV-2 Omicron Infection, N Engl J Med 2022; 387:1333-1336, DOI: 10.1056/NEJMc2209651; Sano, K., Bhavsar, D., Singh, G. et al. SARS-CoV-2 vaccination induces mucosal antibody responses in previously infected individuals. Nat Commun 13, 5135 (2022). https://doi.org/10.1038/s41467-022-32389-8; Yannick G. et al. Humoral Responses and Serological Assays in SARS-CoV-2 Infections, Frontiers in Immunology, Vol 11 (2020) 10.3389/fimmu.2020.610688; Forgacs David et al., SARS-CoV-2 mRNA Vaccines Elicit Different Responses in Immunologically Naïve and Pre-Immune Humans; Front. Immunol., Vol 12 (27 Sep. 2021) https://doi.org/10.3389/fimmu.2021.728021, the entire contents of each of which is incorporated by reference herein for all purposes.

### 5.21.6 Methods of Increasing the Level of Immunogen-Specific IgG

In one aspect, provided herein are methods of increasing the level of immunogen-specific IgG (e.g., mucosal IgG, circulating IgG) in a subject in need thereof, the method comprising administering to the subject (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby increase the level of immunogen-specific IgG (e.g., mucosal IgG, circulating IgG) in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of increasing the level of immunogen-specific IgG (e.g., mucosal IgG, circulating IgG) in a subject in need thereof, the method comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby increase the level of immunogen-specific IgG (e.g., mucosal IgG, circulating IgG) in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of increasing the level of immunogen-specific IgG (e.g., mucosal IgG, circulating IgG) in a subject, the method comprising administering to the subject (b) in combination with a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby increase the level of immunogen-specific IgG (e.g., mucosal IgG, circulating IgG) in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of increasing the level of immunogen-specific IgG (e.g., mucosal IgG, circulating IgG) in a subject, the method comprising administering (a) in combination with (b) to the subject, to thereby increase the level of immunogen-specific IgG (e.g., mucosal IgG, circulating IgG) in a subject.

Provided herein is a combination composition described herein or combination therapy described herein for use in a method of increasing the level of immunogen-specific IgG (e.g., mucosal IgG, circulating IgG) in a subject, the method comprising administering to the subject the combination composition described herein, or the combination therapy described herein, to thereby increase the level of immunogen-specific IgG (e.g., mucosal IgG, circulating IgG) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), for use in a method of increasing the level of immunogen-specific IgG (e.g., mucosal IgG, circulating IgG) in a subject, the method comprising administering (a) in combination with (b) to the subject, to thereby increase the level of immunogen-specific IgG (e.g., mucosal IgG, circulating IgG) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of increasing the level of immunogen-specific IgG (e.g., mucosal IgG, circulating IgG) in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby increase the level of immunogen-specific mucosal IgG in a subject.

Provided herein is a combination composition described herein or combination therapy described herein for the manufacture of a medicament for use in a method of increasing the level of immunogen-specific IgG (e.g., mucosal IgG, circulating IgG) in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In one aspect, provided herein are methods of increasing the level of immunogen-specific IgG (e.g., mucosal IgG, circulating IgG) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (b) a hIL-10R binding protein e.g., comprising, (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby increase the level of immunogen-specific IgG in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of increasing the level of immunogen-specific IgG (e.g., mucosal IgG, circulating IgG) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby increasing the level of immunogen-specific IgG (e.g., mucosal IgG, circulating IgG) in a subject vaccinated with (a) at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of increasing the level of immunogen-specific IgG (e.g., mucosal IgG, circulating IgG) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (a) in combination with (b), to thereby increase the level of immunogen-specific IgG (e.g., mucosal IgG, circulating IgG) in a subject vaccinated with (a) at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of increasing the level of immunogen-specific IgG (e.g., mucosal IgG, circulating IgG) in a subject vaccinated with (a) at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

In some embodiments, the immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

For the sake of clarity it is to be understood that the following embodiments are applicable to any of the foregoing aspects.

In some embodiments, (b) is administered to the subject in an amount and for a time sufficient to increase the level of immunogen specific IgG in the subject.

In some embodiments, the level of immunogen specific IgG is increased by at least about 1-fold, 10-fold, 100-fold, 1,000-fold, or 10,000-fold.

In some embodiments, the level of immunogen specific IgG antibodies is increased by at least about 5%, 10%, 15%, 20%, 25%, 30%, 35%, 40%, 45%, 50%, 55%, 60%, 65%, 70%, 75%, 80%, 85%, 90%, or 95%, or more (e.g., relative to a control composition that does not contain the hIL-10R binding agent (e.g., a hIL-10R binding protein (or a functional fragment and/or functional variant thereof). In some embodiments, the level of immunogen specific mucosal IgA antibodies is increased from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

In some embodiments, the method further comprises administering the immunogen (e.g., (a)(i)-(vi)) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent (e.g., (b)(i)-(vi)) is administered intranasally and the at least a first dose of the immunogen (e.g., (a)(i)-(vi)) is administered intramuscularly or subcutaneously.

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

5.21.7 Methods of Promoting, Enhancing, and/or Sustaining Plasma Cell Populations

In one aspect, provided herein are methods of promoting the generation of, enhancing the generation of, and/or sustaining the level of plasma cells (e.g., immunogen specific plasma cells (e.g., long lived plasma cells)) in a subject in a subject in need thereof, the method comprising administering to the subject (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby promote the generation of, enhance the generation of, and/or sustain the level of plasma cells (e.g., immunogen specific plasma cells (e.g., long lived plasma cells)) in the subject.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of promoting the generation of, enhancing the generation of, and/or sustaining the level of plasma cells (e.g., immunogen specific plasma cells (e.g., long lived plasma cells)) in a subject in need thereof, the method comprising administering to the subject (a) an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), in combination with (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), to thereby promote the generation of, enhance the generation of, and/or sustain the level of plasma cells (e.g., immunogen specific plasma cells (e.g., long lived plasma cells)) in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of promoting the generation of, enhancing the generation of, and/or sustaining the level of plasma cells (e.g., immunogen specific plasma cells (e.g., long lived plasma cells)) in a subject, the method comprising administering to the subject (b) in combination with (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby promote the generation of, enhance the generation of, and/or sustain the level of plasma cells (e.g., immunogen specific plasma cells (e.g., long lived plasma cells)) in a subject.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of promoting the generation of, enhancing the generation of, and/or sustaining the level of plasma cells (e.g., immunogen specific plasma cells (e.g., long lived plasma cells)) in a subject, the method comprising administering (a) and (b) to the subject, to thereby promote the generation of, enhance the generation of, and/or sustain the level of plasma cells (e.g., immunogen specific plasma cells (e.g., long lived plasma cells)) in a subject.

Provided herein is a combination composition described herein or combination therapy described herein for use in a method of promoting the generation of, enhancing the generation of, and/or sustaining the level of plasma cells (e.g., immunogen specific plasma cells (e.g., long lived plasma cells)) in a subject, the method comprising administering to the subject the combination composition described herein, or the combination therapy described herein, to thereby promote the generation of, enhance the generation of, and/or sustain the level of plasma cells (e.g., immunogen specific plasma cells (e.g., long lived plasma cells)) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of promoting the generation of, enhancing the generation of, and/or sustaining the level of plasma cells (e.g., immunogen specific plasma cells (e.g., long lived plasma cells)) in a subject, the method comprising administering (a) in combination with (b) to the subject, to thereby promote the generation of, enhance the generation of, and/or sustain the level of plasma cells (e.g., immunogen specific plasma cells (e.g., long lived plasma cells)) in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of promoting the generation of, enhancing the generation of, and/or sustaining the level of plasma cells (e.g., immunogen specific plasma cells (e.g., long lived plasma cells)) in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby promote the generation of, enhance the generation of, and/or sustain the level of plasma cells (e.g., immunogen specific plasma cells (e.g., long lived plasma cells)) in a subject.

Provided herein is a combination composition described herein or combination therapy described herein for the manufacture of a medicament for use in a method of promoting the generation of, enhancing the generation of, and/or sustaining the level of plasma cells (e.g., immunogen specific plasma cells (e.g., long lived plasma cells)) in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In one aspect, provided herein are methods of promoting the generation of, enhancing the generation of, and/or sustaining the level of plasma cells (e.g., immunogen specific plasma cells (e.g., long lived plasma cells)) in a subject that has been vaccinated against the infection with at least a first dose of an immunogen the method comprising administering to the subject (a) a hIL-10R binding agent comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), to thereby promote the generation of, enhance the generation of, and/or sustain the level of plasma cells (e.g., immunogen specific plasma cells (e.g., long lived plasma cells)) in the subject. In some embodiments, the (a) hIL-10R binding agent is administered for an amount of time and at a dose sufficient to promote the generation of, enhance the generation of, and/or sustain the level of plasma cells (e.g., immunogen specific plasma cells (e.g., long lived plasma cells) in the subject.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of promoting the generation of, enhancing the generation of, and/or sustaining the level of plasma cells (e.g., immunogen specific plasma cells (e.g., long lived plasma cells)) in a subject that has been vaccinated against an infection with at least a first dose of an immunogen, the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby promote the generation of, enhance the generation of, and/or sustain the level of plasma cells (e.g., immunogen specific plasma cells (e.g., long lived plasma cells)) in a subject that has been vaccinated against an infection with at least a first dose of an immunogen.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of promoting the generation of, enhancing the generation of, and/or sustaining the level of plasma cells (e.g., immunogen specific plasma cells (e.g., long lived plasma cells)) in a subject that has been vaccinated against an infection with at least a first dose of an immunogen, the method comprising administering (a) in combination with (b) to the subject, to thereby promote the generation of, enhance the generation of, and/or sustain the level of plasma cells (e.g., immunogen specific plasma cells (e.g., long lived plasma cells)) in a subject that has been vaccinated against an infection with at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of promoting the generation of, enhancing the generation of, and/or sustaining the level of plasma cells (e.g., immunogen specific plasma cells (e.g., long lived plasma cells)) in a subject that has been vaccinated against an infection with at least a first dose of an immunogen.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of promoting the generation of, enhancing the generation of, and/or sustaining the level of plasma cells (e.g., immunogen specific plasma cells (e.g., long lived plasma cells)) in a subject that has been vaccinated against an infection with at least a first dose of an immunogen.

In one aspect, provided herein are methods of promoting the generation of, enhancing the generation of, and/or sustaining the level of plasma cells (e.g., immunogen specific plasma cells (e.g., long lived plasma cells)) in a subject that has received at least a first dose of a vaccine regimen against an infective agent (e.g., a pathogen) or a tumor the method comprising administering to the subject (a) a hIL-10R binding agent comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), to thereby promote the generation of, enhance the generation of, and/or sustain the level of plasma cells (e.g., immunogen specific plasma cells (e.g., long lived plasma cells)) in the subject. In some embodiments, the (a) hIL-10R binding agent is administered for an amount of time and at a dose sufficient to promote the generation of, enhance the generation of, and/or sustain the level of plasma cells (e.g., immunogen specific plasma cells (e.g., long lived plasma cells) in the subject.

For the sake of clarity it is to be understood that the following embodiments are applicable to any of the foregoing aspects.

In some embodiments, the plasma cells are immunogenic specific plasma cells. In some embodiments, the plasma cells are long lived plasma cells. In some embodiments, the plasma cells are immunogen specific long lived plasma cells.

Plasma cells and subpopulations thereof (e.g., long lived plasma cells, plasmablasts, etc.) can be identified and quantified in vitro through standard methods in the art, e.g., through the analysis of proteins expressed on the surface of B cells. See, e.g., Example 4 herein. See, also, Giannotta G, Giannotta N. mRNA COVID-19 Vaccines and Long-Lived Plasma Cells: A Complicated Relationship. Vaccines (Basel). 2021 Dec. 20; 9(12):1503. doi: 10.3390/vaccines9121503. PMID: 34960249; PMCID: PMC8703557, the entire contents of which are incorporated by reference herein for all purposes.

In some embodiments, the plasma cells (e.g., immunogen specific plasma cells (e.g., long lived plasma cells) are detectable in a sample (e.g., blood sample) obtained from the subject at least 3 months, 6 months, 9 months, 12 months, or longer after the administration of the hIL-10R binding agent. In some embodiments, the plasma cells (e.g., immunogen specific plasma cells (e.g., long lived plasma cells) are detectable in a sample (e.g., blood sample) obtained from the subject at least 3 months, 6 months, 9 months, 12 months, 16 month, 20 months, 24 months, 3 years, 4 years, 5 years, 6 years, 7 years, 8 years, 9 years, 10 years or longer after the administration of the hIL-10R binding agent.

In some embodiments, the level of plasma cells (e.g., immunogen specific plasma cells (e.g., long lived plasma cells) is increased by at least about 5%, 10%, 15%, 20%, 25%, 30%, 35%, 40%, 45%, 50%, 55%, 60%, 65%, 70%, 75%, 80%, 85%, 90%, or 95%, or more (e.g., relative to a control composition that does not contain the hIL-10R binding agent (e.g., a hIL-10R binding protein (or a functional fragment and/or functional variant thereof). In some embodiments, the level of plasma cells (e.g., immunogen specific plasma cells (e.g., long lived plasma cells) is increased from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

In some embodiments, the first dose of the vaccine regimen against the infective agent (e.g., pathogen) or the tumor comprises (b) an immunogen (e.g., from the infective agent (e.g., the pathogen) or the tumor) comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the (a) hIL-10R binding agent is administered in combination with a dose of the vaccine regimen. In some embodiments, the dose of the vaccine regimen comprises (b) an immunogen (e.g., from the infective agent (e.g., the pathogen) or the tumor) comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, the hIL-10R binding agent (e.g., (a)(i)-(vi)) is administered as a booster in a prime-boost regimen. In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (a)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of a vaccine regimen to the subject.

In some embodiments, the hIL-10R binding agent (e.g., (a)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of a vaccine regimen to the subject.

In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of a vaccine regimen to the subject. In some embodiments, the hIL-10R binding agent (e.g., (a)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of a vaccine regimen to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of a vaccine regimen to the subject. In some embodiments, the hIL-10R binding agent (e.g., (a)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of a vaccine regimen to the subject.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the hIL-10R binding agent (e.g., (b)(i)-(vi)).

### 5.21.8 Methods of Modulating (e.g., Preventing, Ameliorating, Reducing) Vaccine Reactogenicity

Provided herein are, inter alia, methods of preventing, ameliorating, or reducing reactogenicity induced by a vaccine, the method comprising administering to the subject (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce the reactogenicity of a vaccine.

In some embodiments, the subject has been vaccinated (e.g., against an infection) with at least a first dose of an immunogen (e.g., comprising (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (ii), (iv) a carrier comprising any one or more of (i)-(iii), (v) a composition comprising any one of (i)-(iv), or (vi) a pharmaceutical composition comprising any one of (i)-(v)). In some embodiments, the subject is at least partially vaccinated. In some embodiments, the method comprises simultaneously vaccinating the subject (e.g., with a first dose of an immunogen, a second dose of an immunogen, etc.).

In some embodiments, the method comprises administering to the subject an immunogen (e.g., an immunogenic protein (or an immunogenic fragment and/or immunogenic variant thereof) or a nucleic acid molecule comprising a coding region encoding an immunogenic protein (or an immunogenic fragment and/or immunogenic variant thereof)), in combination with the hIL-10R binding protein (or the functional fragment and/or functional variant thereof) or the nucleic acid molecule comprising a coding region encoding a hIL-10R binding protein (or the functional fragment and/or functional variant thereof).

In one aspect, provided herein are methods of preventing, ameliorating, or reducing reactogenicity induced by a vaccine, the method comprising administering to the subject a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); in combination with (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce reactogenicity of the vaccine.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or reducing reactogenicity induced by a vaccine in a subject, the method comprising administering to the subject (b) in combination with administering to the subject a vaccine comprising (a) an immunogen (e.g., from the infective agent (e.g., the pathogen) or the tumor) e.g., comprising (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v); to thereby reduce reactogenicity of the vaccine.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)); and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)), for use in a method of preventing, ameliorating, or reducing reactogenicity induced by a vaccine in a subject, the method comprising administering (a) in combination with (b) to the subject, to thereby prevent, ameliorate, or reduce reactogenicity induced by a vaccine in a subject.

Provided herein is a combination composition described herein or combination therapy described herein for use in a method of preventing, ameliorating, or reducing reactogenicity induced by a vaccine in a subject, the method comprising administering to the subject the combination composition described herein, or the combination therapy described herein, to thereby prevent, ameliorate, or reduce reactogenicity induced by a vaccine in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of preventing, ameliorating, or reducing reactogenicity induced by a vaccine in a subject, the method comprising administering (a) in combination with (b) to the subject, to thereby preventing, ameliorating, or reducing reactogenicity induced by a vaccine in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or reducing reactogenicity induced by a vaccine in a subject, wherein the medicament is administered to the subject in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby prevent, ameliorate, or reduce reactogenicity induced by a vaccine in a subject.

Provided herein is a combination composition described herein or combination therapy described herein for the manufacture of a medicament for use in a method of preventing, ameliorating, or reducing reactogenicity induced by a vaccine in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, the immunogen (e.g., (a)(i)-(vi)) and the hIL-10R binding agent (e.g., (b)(i)-(vi)) are administered to the subject as part of a prime-boost regimen. In some embodiments, the immunogen (e.g., (a)(i)-(vi)) is administered to the subject as a prime vaccine and the hIL-10R binding agent (e.g., (b)(i)-(vi)) is administered to the subject as a prime vaccine. In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine.

In some embodiments, the immunogen (e.g., (a)(i)-(vi)) is administered to the subject as a prime vaccine and the hIL-10R binding agent (e.g., (b)(i)-(vi)) is subsequently administered to the subject as a booster vaccine, wherein the hIL-10R binding agent (e.g., (b)(i)-(vi)) is administered in combination with an immunogen. In some embodiments, the immunogen is the same as the immunogen administered in (a). In some embodiments, the immunogen is different from the immunogen administered in (a). In some embodiments, the immunogen is from the same infectious agent as the immunogen administered in (a). In some embodiments, the immunogen is a variant of the immunogen administered in (a).

In some embodiments, (a) and (b) are administered concurrently. In some embodiments, (b) is administered before (a).

In some embodiments, (b) is administered after (a). In some embodiments, after is from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month.

In some embodiments, after is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

In some embodiments, after is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, after is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, after is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, after is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

In one aspect, provided herein are methods of preventing, ameliorating, or reducing reactogenicity induced by a vaccine, the method comprising (a) first administering to the subject a vaccine comprising at least a first dose of an immunogen (e.g., from the infective agent (e.g., the pathogen) or the tumor) e.g., comprising, (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), and (b) thereafter administering to the subject a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce the reactogenicity of the vaccine.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or reducing reactogenicity induced by a vaccine in a subject, the method comprising (a) first administering to the subject a vaccine comprising at least a first dose of an immunogen (e.g., from the infective agent (e.g., the pathogen) or the tumor) e.g., comprising, (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v), and (b) thereafter administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce the reactogenicity of the vaccine.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or reducing reactogenicity induced by a vaccine in a subject, the method comprising first administering (a) and thereafter administering (b) to the subject, to thereby prevent, ameliorate, or reduce reactogenicity induced by a vaccine in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in combination with (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) for use in a method of preventing, ameliorating, or reducing reactogenicity induced by a vaccine in a subject, the method comprising first administering (a) and thereafter administering (b) to the subject, to thereby preventing, ameliorating, or reducing reactogenicity induced by a vaccine in a subject.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or reducing reactogenicity induced by a vaccine in a subject, wherein the medicament is administered to the subject after administration of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby prevent, ameliorate, or reduce reactogenicity induced by a vaccine in a subject.

In some embodiments, the (a) immunogen comprises (i) an immunogenic protein (e.g., described herein) (or an immunogenic fragment or variant thereof) (e.g., as a prime), (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v).

In some embodiments, the (b) hIL-10R binding agent comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)).

In some embodiments, thereafter is from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month.

In some embodiments, thereafter is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days. In some embodiments, thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

In one aspect, provided herein are methods of methods of preventing, ameliorating, or reducing the reactogenicity induced by a vaccine in a subject vaccinated with a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject (b) a hIL-10R binding protein comprising, e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce the reactogenicity of the vaccine.

Provided herein is (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for use in a method of preventing, ameliorating, or reducing reactogenicity induced by a vaccine in a subject vaccinated with a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering to the subject the hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v), to thereby reduce the reactogenicity of the vaccine.

Provided herein is a combination of (a) a vaccine comprising an immunogen (e.g., from the infective agent (e.g., the pathogen) or the tumor) (e.g., (i) an immunogenic protein (e.g., described herein) (or an immunogenic fragment or variant thereof) (ii) a nucleic acid molecule comprising a coding region encoding an immunogenic protein (e.g., described herein) (or an immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)) and (b) a hIL-10R binding agent (e.g., (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v)) for use in a method of preventing, ameliorating, or reducing reactogenicity induced by a vaccine in a subject vaccinated with a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), the method comprising administering (a) in combination with (b) to the subject, to thereby prevent, ameliorate, or reduce reactogenicity induced by a vaccine in a subject vaccinated with a vaccine.

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or reducing reactogenicity induced by a vaccine in a subject, the method comprising administering (b) to the subject, to thereby preventing, ameliorating, or reducing reactogenicity induced by a vaccine in a subject vaccinated with a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

Provided herein is a use of (b) a hIL-10R binding agent e.g., comprising (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v) for the manufacture of a medicament for use in a method of preventing, ameliorating, or reducing reactogenicity induced by a vaccine in a subject, wherein the medicament is administered to the subject after administration of (a) a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)), to thereby prevent, ameliorate, or reduce reactogenicity induced by a vaccine in a subject vaccinated with a vaccine (e.g., comprising (a) at least a first dose of an immunogen e.g., comprising, e.g., (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

In some embodiments, the (a) immunogen comprises (i) an immunogenic protein (e.g., described herein) (or an immunogenic fragment or variant thereof), (ii) a nucleic acid molecule comprising a coding region encoding the immunogenic protein (e.g., described herein) (or the immunogenic fragment or variant thereof), (iii) a vector comprising the nucleic acid molecule of (a)(ii), (iv) a carrier comprising any one or more of (a)(i)-(iii), (v) a composition comprising any one of (a)(i)-(iv), or (vi) a pharmaceutical composition comprising any one of (a)(i)-(v)).

In some embodiment, the (b) hIL-10R binding protein comprises (i) a hIL-10R binding protein (e.g., described herein) (or a functional fragment or variant thereof) (or a fusion protein or conjugate thereof), (ii) a nucleic acid molecule comprising a coding region encoding a hIL-10R binding protein (e.g., described herein) (or a functional fragment or variant thereof) (or a fusion protein or conjugate thereof), (iii) a vector comprising the nucleic acid molecule of (b)(ii), (iv) a carrier comprising any one or more of (b)(i)-(iii), (v) a composition comprising any one of (b)(i)-(iv), or (vi) a pharmaceutical composition comprising any one of (b)(i)-(v).

In some embodiments, (b) is administered to the subject in an amount and for a time sufficient to prevent, ameliorate, or reduce the reactogenicity induced by the vaccine in the subject.

In some embodiments, the method further comprises administering the immunogen (e.g., (a)(i)-(vi)) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (e.g., (b)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent (e.g., (b)(i)-(vi)) is administered intranasally and the at least a first dose of the immunogen (e.g., (a)(i)-(vi)) is administered intramuscularly or subcutaneously.

In some embodiments, the hIL-10R binding agent (e.g., (b)(i)-(vi)) is administered to the subject from about 24 hours and 12 months, e.g., 24 hours and 11 months, 24 hours and 10 months, 24 hours and 9 months, 24 hours and 8 months, 24 hours and 7 months, 24 hours and 6 months, 24 hours and 5 months, 24 hours and 4 months, 24 hours and 3 months, 24 hours and 2 months, 24 hours and 1 month, 24 hours and 3 weeks, 24 hours and 2 weeks, 24 hours and 1 week, 48 hours and 11 months, 48 hours and 10 months, 48 hours and 9 months, 48 hours and 8 months, 48 hours and 7 months, 48 hours and 6 months, 48 hours and 5 months, 48 hours and 4 months, 48 hours and 3 months, 48 hours and 2 months, 48 hours and 1 month, 48 hours and 3 weeks, 48 hours and 2 weeks, 48 hours and 1 week, 1 week and 11 months, 1 week and 10 months, 1 week and 9 months, 1 week and 8 months, 1 week and 7 months, 1 week and 6 months, 1 week and 5 months, 1 week and 4 months, 1 week and 3 months, 1 week and 2 months, 1 week and 1 month, 1 week and 3 weeks, 1 week and 2 weeks, 2 weeks and 11 months, 2 weeks and 10 months, 2 weeks and 9 months, 2 weeks and 8 months, 2 weeks and 7 months, 2 weeks and 6 months, 2 weeks and 5 months, 2 weeks and 4 months, 2 weeks and 3 months, 2 weeks and 2 months, 2 weeks and 1 month, 2 weeks and 3 weeks, 3 weeks and 2 months, 3 weeks and 11 months, 3 weeks and 10 months, 3 weeks and 9 months, 3 weeks and 8 months, 3 weeks and 7 months, 3 weeks and 6 months, 3 weeks and 5 months, 3 weeks and 4 months, 3 weeks and 3 months, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month after administration of the at least a first dose of the immunogen (e.g., (a)(i)-(vi)) to the subject.

In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, 90 days, 180 days, or 365 days after administration of the at least a first dose of the immunogen (e.g., (a)(i)-(vi)) to the subject. In some embodiments, the hIL-10R binding agent (e.g., (b)(i)-(vi)) is administered to the subject at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen (e.g., (a)(i)-(vi)) to the subject.

For the sake of clarity, the following embodiments are described in relation to any of the foregoing methods in this § 5.21.8.

(a) and (b) can be administered to the subject by any route (e.g., described herein, see, e.g., § 5.20). In some embodiments the first administering of (a) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments, the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))). In some embodiments the first administering of (a) comprises parenteral administration (e.g., intramuscular, intradermal, subcutaneous, transcutaneous, or mucosal administration, (e.g., inhalation, intranasal, oral)); and the thereafter administering of (b) comprises parenteral administration (e.g., intramuscular, intradermal, subcutaneous, transcutaneous, or mucosal administration, (e.g., inhalation, intranasal, oral, administration into the ear (or a specific compartment thereof (e.g., the middle ear, inner ear, etc.))).

Non-limiting embodiments include parenteral administration, such as intramuscular, intradermal, subcutaneous, transcutaneous, or mucosal administration, e.g., inhalation, intranasal, oral, into the ear (or a specific compartment thereof (e.g., the middle ear, inner ear, etc.), and the like.

In some embodiments, the first administering of (a) comprises intramuscular, subcutaneous, or intranasal administration. In some embodiments, the thereafter administering of (b) comprises intramuscular, subcutaneous, or intranasal administration. In some embodiments, the first administering of (a) comprises intramuscular, subcutaneous, or intranasal administration and the thereafter administering of (b) comprises intramuscular, subcutaneous, or intranasal administration.

In some embodiments, the first administering of (a) comprises intramuscular or subcutaneous administration. In some embodiments, the thereafter administering of (b) comprises intranasal administration. In some embodiments, the first administering of (a) comprises intramuscular or subcutaneous administration and the thereafter administering of (b) comprises intranasal administration.

In some embodiments, the method further comprises administering the immunogen (e.g., (a)(i)-(vi)) to the subject in combination with the administration of the hIL-10R binding agent (e.g., (b)(i)-(vi)). In some embodiments, the hIL-10R binding agent (e.g., (b)(i)-(vi)) is administered as a booster in a prime-boost regimen, wherein the prime portion of the regimen comprises administration of the at least a first dose of the immunogen (e.g., (a)(i)-(vi)) to the subject; and the boost portion of the regimen comprises the administration of the immunogen (e.g., (a)(i)-(vi)) and the hIL-10R binding agent (i.e., (b)(i)-(vi)). In some embodiments, the boost portion of the regimen is administered intramuscularly, subcutaneously, or mucosally (e.g., intranasally). In some embodiments, the boost portion of the regimen is administered to the mucosa (e.g., nasal mucosa, gastrointestinal mucosa, urogenital mucosa, oral mucosa, ear mucosa, etc.). In some embodiments, the boost portion of the regimen is administered intranasally. In some embodiments, the prime portion of the regimen is administered intramuscularly, subcutaneously, or mucosally (e.g., intranasally). In some embodiments, the prime portion of the regimen is administered intramuscularly or subcutaneously. In some embodiments, the prime portion of the regimen is administered intramuscularly or subcutaneously; and the boost portion of the regimen is administered intranasally.

In some embodiments, the hIL-10R binding agent (e.g., (b)(i)-(vi)) is administered intramuscularly, subcutaneously, or mucosally (e.g., intranasally). In some embodiments, the hIL-10R binding agent (e.g., (b)(i)-(vi)) is administered mucosally (e.g., intranasally). In some embodiments, the hIL-10R binding agent (e.g., (b)(i)-(vi)) is administered intranasally. In some embodiments, the at least a first dose of the immunogen (e.g., (a)(i)-(vi)) is administered intramuscularly, subcutaneously, or mucosally (e.g., intranasally). In some embodiments, the at least a first dose of the immunogen (e.g., (a)(i)-(vi)) is administered intramuscularly or subcutaneously. In some embodiments, the hIL-10R binding agent is administered intranasally and the at least a first dose of the immunogen (e.g., (a)(i)-(vi)) is administered intramuscularly or subcutaneously.

In some embodiments, the method further comprises administering to the subject an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)). In some embodiments, the method further comprises administering to the subject an IGIP protein (or a functional fragment and/or functional variant thereof) (e.g., described herein, see, e.g., § 5.7). In some embodiments, the method further comprises administering to the subject a nucleic acid molecule encoding the IGIP protein (or the functional fragment and/or functional variant thereof) (e.g., described herein, see, e.g., § 5.8)).

In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or after administration of the immunogen (e.g., (a)(i)-(vi)). In some embodiments, the IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)) is administered to the subject prior to, concurrently with, and/or or after administration of the hIL-10R binding agent (e.g., (b)(i)-(vi)).

Vaccine reactogenicity is commonly used to describe signs and symptoms that is associated with the inflammatory response to a vaccination. The signs and symptoms can be divided into both local (e.g., injection-site pain, redness, swelling at the site of injection) and systemic (e.g., fever, nausea, vomiting, diarrhea, headaches, fatigue, arthralgia, and myalgia). See, e.g., Herve, C., Laupeze, B., Del Giudice, G. et al. The how's and what's of vaccine reactogenicity. Npj Vaccines 4, 39 (2019). https://doi.org/10.1038/s41541-019-0132-6, and Lee, J., Woodruff, M. C., Kim, E. H. et al. Knife's edge: Balancing immunogenicity and reactogenicity in mRNA vaccines. Exp Mol Med 55, 1305-1313 (2023). https://doi.org/10.1038/s12276-023-00999-x; the entire contents of each of which are incorporated herein by reference for all purposes.

In some embodiments, the preventing, ameliorating, or reducing the reactogenicity is mediated by the suppression of expression of one or more pro-inflammatory cytokine (including e.g., IFNγ, IL-6, IL-1β, and/or TNF-α). In some embodiments, the preventing, ameliorating, or reducing the reactogenicity is mediated by the suppression of expression of one or more pro-inflammatory cytokine expressed by T cell (e.g., IFNγ). In some embodiments, the preventing, ameliorating, or reducing the reactogenicity is mediated by the suppression of expression of one or more pro-inflammatory cytokine expressed by monocytes (e.g., IL-6, and/or IL-1β).

In some embodiments, the preventing, ameliorating, or reducing the reactogenicity is mediated by the suppression of expression of one or more of IFNγ, IL-6, IL-1β, and/or TNF-α. In some embodiments, the preventing, ameliorating, or reducing the reactogenicity is mediated by the suppression of expression of IFNγ. In some embodiments, the preventing, ameliorating, or reducing the reactogenicity is mediated by the suppression of expression of IL-6. In some embodiments, the preventing, ameliorating, or reducing the reactogenicity is mediated by the suppression of expression of IL-10. In some embodiments, the preventing, ameliorating, or reducing the reactogenicity is mediated by the suppression of expression of TNF-α.

In some embodiments, the level of a proinflammatory cytokine (e.g., a proinflammatory cytokine associated with reactogenicity (e.g., IL-10, IFNγ, IL-6, etc.)) is suppressed by at least about 5%, 10%, 15%, 20%, 25%, 30%, 35%, 40%, 45%, 50%, 55%, 60%, 65%, 70%, 75%, 80%, 85%, 90%, or 95% (e.g., relative to a control composition that does not contain the hIL-10R binding agent (e.g., a hIL-10R binding protein (or a functional fragment and/or functional variant thereof). In some embodiments, the level of a proinflammatory cytokine (e.g., a proinflammatory cytokine associated with reactogenicity (e.g., IL-10, IFNγ, IL-6, etc.)) is suppressed from about 5%-75%, 10%-75%, 15%-75%, 20%-75%, 25%-75%, 30%-75%, 35%-75%, 40%-75%, 45%-75%, 50%-75%, 55%-75%, 60%-75%, 70%-75%, 5%-70%, 10%-70%, 15%-70%, 20%-70%, 25%-70%, 30%-70%, 35%-70%, 40%-70%, 45%-70%, 50%-70%, 55%-70%, 60%-70%, 65%-70%, 5%-65%, 10%-65%, 15%-65%, 20%-65%, 25%-65%, 30%-65%, 35%-65%, 40%-65%, 45%-65%, 50%-65%, 55%-65%, 60%-65%, 5%-60%, 10%-60%, 15%-60%, 20%-60%, 25%-60%, 30%-60%, 35%-60%, 40%-60%, 45%-60%, 50%-60%, 55%-60%, 5%-55%, 10%-55%, 15%-55%, 20%-55%, 25%-55%, 30%-55%, 35%-55%, 40%-55%, 45%-55%, 50%-55%, 5%-50%, 10%-50%, 15%-50%, 20%-50%, 25%-50%, 30%-50%, 35%-50%, 40%-50%, 45%-50%, 5%-45%, 10%-45%, 15%-45%, 20%-45%, 25%-45%, 30%-45%, 35%-45%, 40%-45%, 5%-40%, 10%-40%, 15%-40%, 20%-40%, 25%-40%, 30%-40%, 35%-40%, 5%-35%, 10%-35%, 15%-35%, 20%-35%, 25%-35%, 30%-35%, 5%-30%, 10%-30%, 15%-30%, 20%-30%, 25%-30%, 5%-25%, 10%-25%, 15%-25%, 20%-25%, 5%-20%, 10%-20%, 15%-20%, 5%-15%, 10%-15%, or 5%-10% (e.g., relative to a control composition that does not contain the hIL-10R binding agent (e.g., a hIL-10R binding protein (or a functional fragment and/or functional variant thereof).

Exemplary pro-inflammatory cytokines include those associated with reactogenicity. Exemplary pro-inflammatory cytokines include, e.g., IL-10, IFNγ, IL-6, and TNF-α. In some embodiments, the pro-inflammatory cytokine is IL-10. In some embodiments, the pro-inflammatory cytokine is IFNγ. In some embodiments, the pro-inflammatory cytokine is IL-6. In some embodiments, the pro-inflammatory cytokine is TNF-α.

### 5.22 Kits

In a one aspect, provided herein are kits comprising any agent (e.g., described herein), protein (e.g., described herein) (including e.g., fusion proteins and conjugates), nucleic acid molecule (e.g., described herein), vector (e.g., described herein), composition (e.g., described herein), carrier (e.g., described herein), host cell (e.g., described herein), combination composition (e.g., described herein), combination therapy (e.g., described herein), and/or pharmaceutical composition (e.g., described herein) described herein (e.g., a hIL-10R binding agent; a hIL-10R binding protein (or a functional fragment and/or functional variant thereof) (e.g., described herein, see, e.g., § 5.2) (or a fusion protein or conjugate thereof), (or a nucleic acid molecule encoding the hIL-10R binding protein (or the functional fragment and/or functional variant thereof) (e.g., described herein, see, e.g., § 5.4)) (or a fusion protein or conjugate thereof); an immunogenic protein (or the immunogenic fragment or variant thereof) (e.g., described herein, see, e.g., § 5.5) (or a nucleic acid molecule encoding the immunogenic protein (or the immunogenic fragment or variant thereof) (e.g., described herein, see, e.g., § 5.6)), an IGIP protein (or a functional fragment and/or functional variant thereof) (e.g., described herein, see, e.g., § 5.7) (or a nucleic acid molecule encoding the IGIP protein (or the functional fragment and/or functional variant thereof) (e.g., described herein, see, e.g., § 5.8)), a polycistronic nucleic acid molecule (e.g., described herein (see, e.g., § 5.11)), a combination composition (e.g., described herein, see, e.g., § 5.12)); a vaccine composition (e.g., described herein, see, e.g., § 5.13)); a vector described herein (e.g., described herein, see, e.g., § 5.14)); a carrier described herein (e.g., described herein, see, e.g., § 5.15)), a host cell described herein (e.g., described herein, see, e.g., § 5.18), and/or a pharmaceutical composition (e.g., described herein, see, e.g., § 5.20))). In addition, the kit may comprise a liquid vehicle for solubilizing or diluting, and/or technical instructions. The technical instructions of the kit may contain information about administration and dosage and subject groups.

In some embodiments, the agent (e.g., described herein), protein (e.g., described herein) (including e.g., fusion proteins and conjugates), nucleic acid molecule (e.g., described herein), vector (e.g., described herein), composition (e.g., described herein), carrier (e.g., described herein), host cell (e.g., described herein), combination composition (e.g., described herein), combination therapy (e.g., described herein), and/or pharmaceutical composition (e.g., described herein) described herein is provided in a separate part of the kit. In some embodiments, the agent (e.g., described herein), protein (e.g., described herein) (including e.g., fusion proteins and conjugates), nucleic acid molecule (e.g., described herein), vector (e.g., described herein), composition (e.g., described herein), carrier (e.g., described herein), host cell (e.g., described herein), combination composition (e.g., described herein), combination therapy (e.g., described herein), and/or pharmaceutical composition (e.g., described herein) described herein is optionally lyophilized, spray-dried, or spray-freeze dried. The kit may further contain as a part a vehicle (e.g., buffer solution) for solubilizing the dried or lyophilized any agent (e.g., described herein), protein (e.g., described herein), nucleic acid molecule (e.g., described herein), vector (e.g., described herein), composition (e.g., described herein), carrier (e.g., described herein), host cell (e.g., described herein), combination composition (e.g., described herein), combination therapy (e.g., described herein), and/or pharmaceutical composition (e.g., described herein).

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

Any of the kits described herein may be used in any of the methods described herein (see, e.g., § 5.21).

### 5.23 Exemplary Embodiments

The following provides exemplary embodiments (Es) of the disclosure. The embodiments are exemplary only and are in no way limited.

E1. A combination therapy comprising (a) an immunogenic protein (or an immunogenic fragment and/or immunogenic variant thereof) or a nucleic acid molecule comprising a coding region encoding an immunogenic protein (or an immunogenic fragment and/or immunogenic variant thereof); and (b) a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof).

E2. The combination therapy of Embodiment (E) 1, wherein the combination therapy is utilized in a vaccine regimen.

E3. The combination therapy of E1 or E2, wherein the combination therapy is utilized in a prime-boost vaccine regimen.

E4. The combination therapy of any one of E1-E3, wherein (a) is utilized as a prime vaccine and (b) is utilized as a boost vaccine of the prime-boost vaccine regimen.

E5. The combination therapy of any one of E1-E4, wherein (a) is utilized as a prime vaccine and (b) is utilized as a prime vaccine of the prime-boost vaccine regimen.

E6. The combination therapy of any one of E1-E5, wherein (a) is utilized as a boost vaccine and (b) is utilized as a boost vaccine of the prime-boost vaccine regimen.

E7. The combination therapy of any one of E1-E6, wherein (a) and (b) are administered concurrently or sequentially.

E8. The combination therapy of any one of E1-E7, wherein (a) is administered prior to (b).

E9. The combination therapy of any one of E1-E8, wherein (a) and (b) are not-co-formulated.

E10. The combination therapy of any one of E1-E9, wherein the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in any one of SEQ ID NOS: 188, 179-187, 189-353, or 1-178.

E11. The combination therapy of any one of E1-E10, wherein the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 188.

E12. The combination therapy of any one of E1-E11, wherein the amino acid sequence of the hIL-10R binding protein comprises an amino acid sequence that is at least about 90%, 91%, 92%, 93%, 94%, 95%, 96%, 97%, 98%, 99%, or 100% identical to the amino acid sequence set forth in SEQ ID NO: 10.

E13. The combination therapy of any one of E1-E12, wherein hIL-10R binding protein is operably connected to a heterologous moiety either directly or through a linker (e.g., peptide linker).

E14. The combination therapy of E13, wherein the heterologous moiety comprises an immunoglobulin Fc region.

E15. The combination therapy of any one of E1-E14, wherein (a) comprises an immunogenic protein (or an immunogenic fragment and/or immunogenic variant thereof).

E16. The combination therapy of any one of E1-E15, wherein (a) comprises a nucleic acid molecule encoding an immunogenic protein (or an immunogenic fragment and/or immunogenic variant thereof).

E17. The combination therapy of any one of E1-E16, wherein (b) comprises a hIL-10R binding protein (or a functional fragment and/or functional variant thereof).

E18. The combination therapy of any one of E1-E17, wherein (b) comprises a nucleic acid molecule comprising a coding region encoding a hIL-10R binding protein (or a functional fragment and/or functional variant thereof).

E19. The combination therapy of anyone of E1-E18, wherein the nucleic acid molecule is an RNA molecule.

E20. The combination therapy of E19, wherein the RNA molecule is an mRNA molecule or a circular RNA molecule.

E21. The combination therapy of any one of E1-E20, further comprising an IgA inducing protein (IGIP) protein (or a functional fragment and/or functional variant thereof) or a nucleic acid molecule comprising a coding region encoding the IGIP protein (or the functional fragment and/or functional variant thereof).

E22. The combination therapy of any one of E1-E21, wherein the amino acid sequence of the IGIP protein comprises an amino acid sequence at least about 90%, 91%, 92%, 93%, 94%, 95%, 96%, 97%, 98%, 99%, or 100% identical to the amino acid sequence set forth in any one of SEQ ID NOS: 570-572.

E23. The combination therapy of any one of E1-E22, further comprising an immunogenic SARS-CoV-2 protein (or an immunogenic fragment and/or immunogenic variant thereof) or a nucleic acid molecule comprising a coding region encoding an immunogenic SARS-CoV-2 protein (or an immunogenic fragment and/or immunogenic variant thereof) that is utilized as part of the boost vaccine of the prime-boost vaccine regimen.

E24. The combination therapy of any one of E1-E23, wherein (a) and/or (b) is formulated in a carrier.

E25. The combination therapy of E24, wherein the carrier is a lipid nanoparticle (LNP), liposome, lipoplex, or nanoliposome.

E26. The combination therapy of E25, wherein the carrier is an LNP.

E27. The combination therapy of E26, wherein the LNP comprises a cationic lipid, a neutral lipid, a cholesterol, and/or a PEG lipid.

E28. A vaccine composition (e.g., a vaccine booster composition) comprising (a) a first immunogen (e.g., a first immunogenic protein or a first nucleic acid molecule comprising a coding region encoding the first immunogenic protein), and (b) a human IL-10 Receptor (hIL-10R) binding agent (e.g., a hIL-10R binding protein or a second nucleic acid molecule comprising a coding region encoding the hIL-10R binding protein).

E29. The composition of E28, further comprising an IgA inducing protein (IGIP) (e.g., human IGIP (hIGIP)) protein (e.g., or a third nucleic acid molecule comprising a coding region encoding an IGIP (e.g., hIGIP) protein).

E30. The composition of E29, wherein the IGIP (e.g., hIGIP) protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572 or a protein set forth in Table 13.

E31. The composition of any one of E28-30, wherein the composition comprises (a) the first nucleic acid molecule (e.g., RNA molecule) comprising a coding region encoding the first immunogenic protein; and (b) the second nucleic acid molecule (e.g., RNA molecule) comprising a coding region encoding the hIL-10R binding protein.

E32. The composition of any one of E28-31, wherein the first nucleic acid molecule and the second nucleic acid molecule are comprised in separate nucleic acid molecules.

E33. The composition of any one of E28-32, wherein the first nucleic acid molecule and the second nucleic acid molecule are comprised in a single nucleic acid molecule (i.e., are operably connected).

E34. The composition of any one of E28-33, wherein the first nucleic acid molecule and the second nucleic acid molecule are DNA molecules.

E35. The composition of any one of E28-34, wherein the first nucleic acid molecule and the second nucleic acid molecule are RNA molecules.

E36. The composition of any one of E28-35, wherein the first nucleic acid molecule is an mRNA or a circular RNA; and/or the second nucleic acid molecule is an mRNA or a circular RNA.

E37. The composition of any one of E28-36, wherein the nucleotide sequence of the first nucleic acid molecule comprises at least one modified nucleotide, and/or the nucleotide sequence of the second nucleic acid molecule comprises at least one modified nucleotide.

E38. The composition of any one of E28-37, wherein the nucleotide sequence of the first nucleic acid molecule comprises N1-methyl-pseudouridine, cytosine, adenine, and guanine; and/or the nucleotide sequence of the second nucleic acid molecule comprises N1-methyl-pseudouridine, cytosine, adenine, and guanine.

E39. The composition of any one of E28-38, wherein the first nucleic acid molecule comprises a heterologous 5′-untranslated region (UTR), 3′-UTR, or both a 5′-UTR and 3′-UTR; and/or the second nucleic acid molecule comprises a heterologous 5′-untranslated region (UTR), 3′-UTR, or both a 5′-UTR and 3′-UTR.

E40. The composition of any one of E28-39, wherein the first nucleic acid molecule comprises a poly(A) sequence; and/or the second nucleic acid molecule comprises a poly(A) sequence.

E41. The composition of any one of E28-40, wherein the first nucleic acid molecule comprises a 5′cap structure; and/or the second nucleic acid molecule comprises a 5′cap structure.

E42. The composition of any one of E28-41, wherein the nucleotide sequence of the first nucleic acid molecule is codon optimized; and/or the nucleotide sequence of the second nucleic acid molecule is codon optimized.

E43. The composition of any one of E28-42, further comprising a third nucleic acid molecule comprising a coding region encoding an IGIP (e.g., hIGIP) protein.

E44. The composition of E43, wherein the third nucleic acid molecule is comprised on a separate nucleic acid molecule than the first nucleic acid molecule and the second nucleic acid molecule.

E45. The composition of any one of E43-44, wherein the third nucleic acid molecule is comprised on the same nucleic acid molecule as the first nucleic acid molecule or the second nucleic acid molecule.

E46. The composition of any one of E43-45, wherein the third nucleic acid molecule is comprised on the same nucleic acid molecule as the first nucleic acid molecule and the second nucleic acid molecule.

E47. The composition of any one of E43-46, wherein the third nucleic acid molecule is an RNA or DNA molecule.

E48. The composition of any one of E43-47, wherein the third nucleic acid molecule is an mRNA or a circular RNA.

E49. The composition of any one of E43-48, wherein the nucleotide sequence of the third nucleic acid molecule comprises at least one modified nucleotide.

E50. The composition of any one of E43-49, wherein the nucleotide sequence of the third nucleic acid molecule comprises N1-methyl-pseudouridine, cytosine, adenine, and guanine.

E51. The composition of any one of E43-50, wherein the third nucleic acid molecule comprises a heterologous 5′-untranslated region (UTR), 3′-UTR, or both a 5′-UTR and 3′-UTR.

E52. The composition of any one of E43-51, wherein the third nucleic acid molecule comprises a poly(A) sequence.

E53. The composition of any one of E43-52, wherein the third nucleic acid molecule comprises a 5′cap structure.

E54. The composition of any one of E43-53, wherein the nucleotide sequence of the third nucleic acid molecule is codon optimized.

E55. The composition of any one of E28-54, further comprising a third nucleic acid molecule comprising a coding region encoding a second immunogenic protein.

E56. The composition of E55, wherein the third nucleic acid molecule is comprised on a separate nucleic acid molecule than the first nucleic acid molecule and the second nucleic acid molecule.

E57. The composition of E55 or E56, wherein the third nucleic acid molecule is comprised on the same nucleic acid molecule as the first nucleic acid molecule or the second nucleic acid molecule.

E58. The composition of any one of E55-E57, wherein the third nucleic acid molecule is comprised on the same nucleic acid molecule as the first nucleic acid molecule and the second nucleic acid molecule.

E59. The composition of any one of E55-E58, wherein the third nucleic acid molecule is an RNA or DNA molecule.

E60. The composition of any one of E55-E59, wherein the third nucleic acid molecule is an mRNA or a circular RNA.

E61. The composition of any one of E55-E60, wherein the nucleotide sequence of the third nucleic acid molecule comprises at least one modified nucleotide.

E62. The composition of any one of E55-E61, wherein the nucleotide sequence of the third nucleic acid molecule comprises N1-methyl-pseudouridine, cytosine, adenine, and guanine.

E63. The composition of any one of E55-E62, wherein the third nucleic acid molecule comprises a heterologous 5′-untranslated region (UTR), 3′-UTR, or both a 5′-UTR and 3′-UTR.

E64. The composition of any one of E55-E63, wherein the third nucleic acid molecule comprises a poly(A) sequence.

E65. The composition of any one of E55-E64, wherein the third nucleic acid molecule comprises a 5′cap structure.

E66. The composition of any one of E55-E65, wherein the nucleotide sequence of the third nucleic acid molecule is codon optimized.

E67. The composition of any one of E28-E66, further comprising a fourth nucleic acid molecule comprising a coding region encoding a second immunogenic protein.

E68. The composition of E67, wherein the fourth nucleic acid molecule is comprised on a separate nucleic acid molecule than the first nucleic acid molecule, the second nucleic acid molecule, and the third nucleic acid molecule.

E69. The composition of E68, wherein the fourth nucleic acid molecule is comprised on the same nucleic acid molecule as the first nucleic acid molecule, the second nucleic acid molecule, and/or the third nucleic acid molecule.

E70. The composition of any one of E67-E69, wherein the fourth nucleic acid molecule is an RNA or DNA molecule.

E71. The composition of any one of E67-E70, wherein the fourth nucleic acid molecule is an mRNA or a circular RNA.

E72. The composition of any one of E67-E71, wherein the nucleotide sequence of the fourth nucleic acid molecule comprises at least one modified nucleotide.

E73. The composition of any one of E67-E72, wherein the nucleotide sequence of the fourth nucleic acid molecule comprises N1-methyl-pseudouridine, cytosine, adenine, and guanine.

E74. The composition of any one of E67-E73, wherein the fourth nucleic acid molecule comprises a heterologous 5′-untranslated region (UTR), 3′-UTR, or both a 5′-UTR and 3′-UTR.

E75. The composition of any one of E67-E74, wherein the fourth nucleic acid molecule comprises a poly(A) sequence.

E76. The composition of any one of E67-E75, wherein the fourth nucleic acid molecule comprises a 5′cap structure.

E77. The composition of any one of E67-E76, wherein the nucleotide sequence of the fourth nucleic acid molecule is codon optimized.

E78. The composition of any one of E28-E77, comprising a plurality of (i.e., at least 2) nucleic acid molecules each comprising a coding region encoding an immunogenic protein.

E79. The composition of E78, wherein the plurality comprises or consists of at least 2, 3, 4, 5, 6, 7, 8, 9, 10, 11, 12, 13, 14, 15, 16, 17, 18, 19, 20, or more nucleic acid molecules.

E80. The composition of any one of E78-E79, wherein at least two (e.g., at least 3, 4, 5, 6, 7, 8, 9, or 10, or more) of the encoded immunogenic proteins of the plurality are variants of the same protein.

E81. The composition of any one of E78-E80, wherein at least two (e.g., at least 3, 4, 5, 6, 7, 8, 9, or 10, or more) of the encoded immunogenic proteins of the plurality are derived from different pathogens.

E82. The composition of any one of E78-E81, wherein at least two (e.g., at least 3, 4, 5, 6, 7, 8, 9, or 10, or more) of the encoded immunogenic proteins of the plurality are derived from the same pathogen.

E83. The composition of any one of E78-E82, wherein at least two (e.g., at least 3, 4, 5, 6, 7, 8, 9, or 10, or more) of the encoded immunogenic proteins of the plurality are derived from different strains of the same pathogen.

E84. The composition of any one of E28-E83, wherein the first nucleic acid molecule, the second nucleic acid molecule, the third nucleic acid molecule, the fourth nucleic acid molecule, and/or the plurality of nucleic acid molecules are comprised in one or more vectors.

E85. The composition of E84, wherein the vector is a viral vector.

E86. The composition of E84, wherein the vector is a non-viral vector (e.g., a plasmid).

E87. The composition of any one of E28-E31, wherein the composition comprises (a) the first immunogenic protein; and (b) the hIL-10R binding protein.

E88. The composition of E87, further comprising an IGIP (e.g., hIGIP) protein.

E89. The composition of E88, wherein the IGIP (e.g., hIGIP) protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572 or a protein set forth in Table 13.

E90. The composition of any one of E87-E89, further comprising a second immunogenic protein.

E91. The composition of any one of E87-E90, wherein the first immunogenic protein is a viral immunogenic protein, bacterial immunogenic protein, fungal immunogenic protein, protozoal immunogenic protein, or a tumor associated immunogenic protein.

E92. The composition of any one of E87-E91, wherein the first immunogenic protein is a viral immunogen.

E93. The composition of any one of E87-E92, wherein the first immunogenic protein is a respiratory virus immunogen.

E94. The composition of any one of E87-E93, wherein the first immunogenic protein is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

E95. The composition of any one of E87-E94, wherein the first immunogenic protein is a SARS-CoV-2 spike immunogen (or an immunogenic fragment or immunogenic variant thereof).

E96. The composition of any one of E87-E95, wherein the first immunogenic protein is an influenza hemagglutinin immunogen or an influenza neuraminidase immunogen.

E97. The composition of any one of E87-E96, wherein the first immunogenic protein is an RSV F immunogen or an RSV G immunogen.

E98. The composition of any one of E87-E97, wherein the amino acid sequence of the first and second immunogenic proteins are different.

E99. The composition of any one of E87-E98, wherein the second immunogenic protein is a viral immunogen, bacterial immunogen, fungal immunogen, protozoal immunogen, or a tumor associated immunogen.

E100. The composition of any one of E87-E99, wherein the second immunogenic protein is a viral immunogen.

E101. The composition of any one of E87-E100, wherein the second immunogenic protein is a respiratory virus immunogen.

E102. The composition of any one of E87-E101, wherein the second immunogenic protein is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus immunogen (RSV), a rhinovirus immunogen, a Parvovirus B19, a parainfluenza virus immunogen, or an adenovirus immunogen.

E103. The composition of any one of E87-E102, wherein the second immunogenic protein is a SARS-CoV-2 spike protein (or an immunogenic fragment or immunogenic variant thereof).

E104. The composition of any one of E87-E103, wherein the second immunogenic protein is an influenza hemagglutinin immunogen or a neuraminidase immunogen.

E105. The composition of any one of E87-E104, wherein the second immunogenic protein is an RSV F protein immunogen or an RSV G protein immunogen.

E106. The composition of any one of E87-E105, wherein the first immunogenic protein is a SARS-CoV-2 spike protein (or an immunogenic fragment or immunogenic variant thereof); and the second immunogenic protein is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

E107. The composition of any one of E87-E106, further comprising a plurality of (i.e., at least 2) immunogenic proteins.

E108. The composition of E107, wherein the plurality comprises or consists of at least 2, 3, 4, 5, 6, 7, 8, 9, 10, 11, 12, 13, 14, 15, 16, 17, 18, 19, 20, or more immunogenic proteins.

E109. The composition of any one of E107-E108, wherein at least two (e.g., at least 3, 4, 5, 6, 7, 8, 9, or 10, or more) of the immunogenic proteins of the plurality are variants of the same protein.

E110. The composition of any one of E107-E109, wherein at least two (e.g., at least 3, 4, 5, 6, 7, 8, 9, or 10, or more) of the immunogenic proteins of the plurality are derived from different pathogens.

E111. The composition of any one of E107-E110, wherein at least two (e.g., at least 3, 4, 5, 6, 7, 8, 9, or 10, or more) of the immunogenic proteins of the plurality are derived from the same pathogen.

E112. The composition of any one of E107-E111, wherein at least two (e.g., at least 3, 4, 5, 6, 7, 8, 9, or 10, or more) of the immunogenic proteins of the plurality are derived from different strains of the same pathogen.

E113. The composition of any one of E28-E112, wherein the hIL-10R binding agent (e.g., hIL-10R binding protein) is a hIL-10R agonist.

E114. The composition of any one of E28-E113, wherein the hIL-10R binding agent (e.g., hIL-10R binding protein) comprises IL-10 (or a functional variant or functional fragment thereof).

E115. The composition of any one of E28-E114, wherein the hIL-10R binding agent (e.g., hIL-10R binding protein) comprises human IL-10 (hIL-10) (or a functional variant or functional fragment thereof).

E116. The composition of any one of E28-E115, wherein the hIL-10R binding agent (e.g., hIL-10R binding protein) comprises a viral IL-10 (vIL-10) (or a functional fragment or functional variant thereof).

E117. The composition of any one of E28-E116, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-353 (e.g., SEQ ID NOS: 1-178, SEQ ID NOS: 179-353).

E118. The composition of any one of E28-E117, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-3 or 179-181.

E119. The composition of any one of E28-E118, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 4-178 or 182-353.

E120. The composition of any one of E28-E119, wherein the hIL-10R binding agent (e.g., hIL-10R binding protein) further comprises a homologous or heterologous signal peptide operably connected to the hIL-10R binding agent (e.g., hIL-10R binding protein).

E121. The composition of any one of E28-E120, wherein the hIL-10R binding agent (e.g., hIL-10R binding protein) further comprises a heterologous moiety.

E122. The composition of E121, wherein the heterologous moiety is operably connected to the hIL-10R binding agent (e.g., hIL-10R binding protein) via a linker.

E123. The composition of E122 or E122, wherein the heterologous moiety comprises a heterologous polypeptide (e.g., a half-life extension polypeptide).

E124. The composition of any one of E121-E123, wherein the heterologous polypeptide comprises an immunoglobulin (Ig) Fc region.

E125. The composition of E124, wherein the Ig Fc region comprises at least a portion of a hinge region, a CH2 region, and a CH3 region.

E126. The composition of E124 or E125, wherein the Ig Fc region comprises a hinge region, a CH2 region, and a CH3 region.

E127. The composition of any one E121-E126, wherein the heterologous polypeptide comprises a human immunoglobulin (hIg) Fc region.

E128. The composition of E127, wherein the hIg is a human IgG (hIgG).

E129. The composition of E128, wherein the hIgG is hIgG1 or hIgG4.

E130. The composition of any one of E12-E124, wherein the heterologous polypeptide comprises a murine immunoglobulin (mIg) Fc region.

E131. The composition of E130, wherein the mIg is a mIgG1.

E132. The composition of E131, wherein the mIg is a mIgG2a.

E133. The composition of any one of E124-E132, wherein the Ig (e.g., hIg, mIg) Fc region comprises one or more amino acid substitutions relative to a reference Ig (e.g., hIg, mIg) Fc region that reduces or abolishes one or more of the following effector functions relative to the reference Ig (e.g., hIg, mIg) Fc region: antibody dependent cell mediated cytotoxicity (ADCC), complement dependent cytotoxicity (CDC), and/or affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E134. The composition of any one of E124-E133, wherein the Ig (e.g., hIg, mIg) Fc region does not substantially mediate ADCC, does not substantially mediate CDC, and/or does not bind to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E135. The composition of any one of E124-E134, wherein the first immunogen (e.g., the first immunogenic protein or the first nucleic acid molecule comprising a coding region encoding the first immunogenic protein)), the hIL-10R binding agent (e.g., the hIL-10R binding protein or the second nucleic acid molecule comprising a coding region encoding the hIL-10R binding protein), the IGIP (e.g., hIGIP) protein (or the third nucleic acid molecule comprising a coding region encoding the IGIP (e.g., hIGIP) protein); the second immunogen (e.g., the second immunogenic protein or the third or fourth nucleic acid molecule comprising a coding region encoding the second immunogenic protein), the plurality of immunogens (or plurality of nucleic acid molecules each encoding an immunogen) and/or the one or more vectors are formulated in one or more carrier.

E136. The composition of E135, wherein the carrier is a lipid nanoparticle (LNP), liposome, lipoplex, or nanoliposome.

E137. The composition of E136, wherein the carrier is an LNP.

E138. The composition of E136 or E137, wherein the LNP comprises a cationic lipid, a neutral lipid, a cholesterol, and/or a PEG lipid.

E139. The composition of any one of E136-E138, wherein the LNP has a mean particle size of between 80 nm and 160 nm.

E140. The composition of any one of E28-E139, wherein the composition is a pharmaceutical composition comprising a pharmaceutically acceptable excipient.

E141. An RNA molecule comprising a coding region encoding a first immunogen (e.g., a first immunogenic protein) and a coding region encoding a hIL-10R binding agent (e.g., a hIL-10R binding protein).

E142. The RNA molecule of E141, further comprising a coding region encoding an IGIP (e.g., hIGIP) protein.

E143. The RNA molecule of E142, wherein the encoded IGIP (e.g., hIGIP) protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572 or a protein set forth in Table 13.

E144. The RNA molecule of any one of E141-E143, wherein the RNA molecule is a messenger RNA (mRNA) or a circular RNA.

E145. The RNA molecule of any one of E141-E144, wherein the nucleotide sequence of the RNA molecule comprises at least one modified nucleotide.

E146. The RNA molecule of any one of E141-E145, wherein the nucleotide sequence of the RNA molecule comprises N1-methyl-pseudouridine, cytosine, adenine, and guanine.

E147. The RNA molecule of any one of E141-E146, wherein the RNA molecule comprises a heterologous 5′-untranslated region (UTR), 3′-UTR, or both a 5′-UTR and 3′-UTR.

E148. The RNA molecule of any one of E141-E147, wherein the RNA molecule comprises a poly(A) sequence.

E149. The RNA molecule of any one of E141-E148, wherein the RNA molecule comprises a 5′cap structure.

E150. The RNA molecule of any one of E141-E149, wherein the nucleotide sequence of the RNA molecule is codon optimized.

E151. The RNA molecule of any one of E141-E150, wherein the first immunogen is a viral immunogen, bacterial immunogen, fungal immunogen, protozoal immunogen, or a tumor associated immunogen.

E152. The RNA molecule of any one of E141-E151, wherein the first immunogen is a viral immunogen.

E153. The RNA molecule of any one of E141-E152, wherein the first immunogen is a respiratory virus immunogen.

E154. The RNA molecule of any one of E141-E153, wherein the first immunogen is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

E155. The RNA molecule of any one of E141-E154, wherein the first immunogen is a SARS-CoV-2 spike protein (or an immunogenic fragment or immunogenic variant thereof).

E156. The RNA molecule of any one of E141-E155, wherein the first immunogen is an influenza hemagglutinin immunogen or an influenza neuraminidase immunogen.

E157. The RNA molecule of any one of E141-E156, wherein the first immunogen is an RSV F protein immunogen or an RSV G protein immunogen.

E158. The RNA molecule of any one of E141-E157, wherein the RNA molecule further comprises a coding region encoding a second immunogen.

E159. The RNA molecule of E158, wherein the amino acid sequence of the first immunogen and the amino acid sequence of the second immunogen are different.

E160. The RNA molecule of E158 or E159, wherein the second immunogen is a viral immunogen, bacterial immunogen, fungal immunogen, protozoal immunogen, or a tumor associated immunogen.

E161. The RNA molecule of any one of E158-E160, wherein the second immunogen is a viral immunogen.

E162. The RNA molecule of any one of E158-E161, wherein the second immunogen is a respiratory virus immunogen.

E163. The RNA molecule of any one of E158-E162, wherein the second immunogen is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

E164. The RNA molecule of any one of E158-E163, wherein the second immunogen is a SARS-CoV-2 spike protein (or an immunogenic fragment or immunogenic variant thereof).

E165. The RNA molecule of any one of E158-E164, wherein the second immunogen is an influenza hemagglutinin immunogen or an influenza neuraminidase immunogen.

E166. The RNA molecule of any one of E158-E165, wherein the second immunogen is an RSV F protein immunogen or an RSV G protein immunogen.

E167. The RNA molecule of any one of E158-E166, wherein the first immunogen is a SARS-CoV-2 spike protein (or an immunogenic fragment or immunogenic variant thereof); and the second immunogen is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV)immunogen, a rhinovirus immunogen, a parvovirus B19 immunogen, a parainfluenza virus immunogen, or an adenovirus immunogen.

E168. The RNA molecule of any one of E141-E167, comprising a plurality of coding regions each encoding an immunogen (e.g., an immunogenic protein).

E169. The RNA molecule of E168, wherein the plurality comprises or consists of at least 2, 3, 4, 5, 6, 7, 8, 9, 10, 11, 12, 13, 14, 15, 16, 17, 18, 19, 20, or more coding regions each encoding an immunogen (e.g., an immunogenic protein).

E170. The RNA molecule of any one of E168-E169, wherein at least two (e.g., at least 3, 4, 5, 6, 7, 8, 9, or 10, or more) of the encoded immunogenic proteins of the plurality are variants of the same immunogen.

E171. The RNA molecule of any one of E168-E170, wherein at least two (e.g., at least 3, 4, 5, 6, 7, 8, 9, or 10, or more) of the encoded immunogenic proteins of the plurality are derived from different pathogens.

E172. The RNA molecule of any one of E168-E171, wherein at least two (e.g., at least 3, 4, 5, 6, 7, 8, 9, or 10, or more) of the encoded immunogenic proteins of the plurality are derived from the same pathogen.

E173. The RNA molecule of any one of E168-E172, wherein at least two (e.g., at least 3, 4, 5, 6, 7, 8, 9, or 10, or more) of the encoded immunogenic proteins of the plurality are derived from different strains of the same pathogen.

E174. The RNA molecule of any one of E141-E173, wherein the hIL-10R binding agent (e.g., hIL-10R binding protein) is a hIL-10R agonist.

E175. The RNA molecule of any one of E141-E174, wherein the hIL-10R binding agent (e.g., hIL-10R binding protein) comprises IL-10 (or a functional variant or functional fragment thereof).

E176. The RNA molecule of any one of E141-E175, wherein the hIL-10R binding agent (e.g., hIL-10R binding protein) comprises hIL-10 (or a functional variant or functional fragment thereof).

E177. The RNA molecule of any one of E141-E176, wherein the hIL-10R binding agent (e.g., hIL-10R binding protein) comprises a vIL-10 (or a functional fragment or functional variant thereof).

E178. The RNA molecule of any one of E141-E177, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-353 (e.g., SEQ ID NOS: 1-178, SEQ ID NOS: 179-353).

E179. The RNA molecule of any one of E141-E178, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-3 or 179-181.

E180. The RNA molecule of any one of E141-E179, wherein the hIL-10R binding agent is a protein that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of 4-178 or 182-353.

E181. The RNA molecule of any one of E141-E180, wherein the hIL-10R binding agent further comprises a homologous or heterologous signal peptide operably connected to the hIL-10R binding agent.

E182. The RNA molecule of any one of E141-E181, wherein the hIL-10R binding agent (e.g., hIL-10R binding protein) further comprises a heterologous moiety.

E183. The RNA molecule of E182, wherein the heterologous moiety is operably connected to the hIL-10R binding agent via a linker.

E184. The RNA molecule of any one of E182-E173, wherein the heterologous moiety comprises a heterologous polypeptide (e.g., a half-life extension polypeptide).

E185. The RNA molecule of any one of E182-E174, wherein the heterologous polypeptide comprises an immunoglobulin (Ig) Fc region.

E186. The RNA molecule of E185, wherein the Ig Fc region comprises at least a portion of a hinge region, a CH2 region, and a CH3 region.

E187. The RNA molecule of any one of E185-186, wherein the Ig Fc region comprises a hinge region, a CH2 region, and a CH3 region.

E188. The RNA molecule of any one of E185-187, wherein the heterologous polypeptide comprises a human immunoglobulin (hIg) Fc region.

E189. The RNA molecule of E188, wherein the hIg is a human IgG (hIgG).

E190. The RNA molecule of E189, wherein the hIgG is hIgG1 or hIgG4.

E191. The RNA molecule of any one of E185-E187, wherein the heterologous polypeptide comprises a murine immunoglobulin (mIg) Fc region.

E192. The RNA molecule of E191, wherein the mIg is a mIgG1.

E193. The RNA molecule of E192, wherein the mIg is a mIgG2a.

E194. The RNA molecule of any one of E185-E193, wherein the Ig (e.g., hIg, mIg) Fc region comprises one or more amino acid substitutions relative to a reference Ig (e.g., hIg, mIg) Fc region that reduces or abolishes one or more of the following effector functions relative to the reference Ig (e.g., hIg, mIg) Fc region: antibody dependent cell mediated cytotoxicity (ADCC), complement dependent cytotoxicity (CDC), and/or affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E195. The RNA molecule of any one of E185-E194, wherein the Ig (e.g., hIg, mIg) Fc region does not substantially mediate ADCC, does not substantially mediate CDC, and/or does not bind to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E196. A vector comprising the RNA molecule of any one of E141-E195.

E197. The vector of E196, wherein the vector is a viral vector.

E198. The vector of E197, wherein the vector is a non-viral vector (e.g., a plasmid).

E199. A carrier comprising the RNA molecule of any one of E141-195 or the vector of any one of E196-198.

E200. The carrier of E199, wherein the carrier is a lipid nanoparticle (LNP), liposome, lipoplex, or nanoliposome.

E201. The carrier of E200, wherein the carrier is an LNP.

E202. The carrier of E201, wherein the LNP comprises a cationic lipid, a neutral lipid, a cholesterol, and/or a PEG lipid.

E203. The carrier of E201 or E202, wherein the LNP has a mean particle size of between 80 nm and 160 nm.

E204. A pharmaceutical composition comprising the combination therapy of any one of E1-27, the vaccine composition of any one of E28-E140, the RNA molecule of any one of E141-195, the vector of any one of E196-E198, and/or the carrier of any one of E199-203; and a pharmaceutically acceptable excipient.

E205. A kit comprising the combination therapy of any one of E1-27, the vaccine composition of any one of E28-E140, the RNA molecule of any one of E141-195, the vector of any one of E196-E198, and/or the carrier of any one of E199-203, and/or the pharmaceutical composition of E204.

E206. The kit of E205, wherein the kit comprises instructions for use of the combination therapy of any one of E1-27, the vaccine composition of any one of E28-E140, the RNA molecule of any one of E141-195, the vector of any one of E196-E198, and/or the carrier of any one of E199-203, and/or the pharmaceutical composition of E204.

E207. A method of vaccinating a subject comprising administering to the subject (a) at least a first dose of an immunogen (e.g., an immunogenic protein or a nucleic acid molecule comprising a coding region encoding the immunogenic protein), in combination with (b) thereafter (e.g., as a booster) administering to the subject a hIL-10R binding agent (e.g., hIL-10R binding protein or a nucleic acid molecule comprising a coding region encoding the hIL-10R binding protein).

E208. A method of vaccinating a subject comprising (a) first administering to the subject at least a first dose of an immunogen (e.g., an immunogenic protein or a nucleic acid molecule comprising a coding region encoding the immunogenic protein), and (b) thereafter (e.g., as a booster) administering to the subject a hIL-10R binding agent (e.g., hIL-10R binding protein or a nucleic acid molecule comprising a coding region encoding the hIL-10R binding protein).

E209. A method of vaccinating a human subject, comprising: (a) first administering to the subject an mRNA vaccine against a pathogen or tumor, and (b) thereafter administering to the human subject a hIL-10R binding agent described herein (e.g., a hIL-10R binding protein (e.g., described herein) or a hIL-10R binding protein encoding mRNA (e.g., described herein)).

E210. A method of vaccinating a human subject, comprising: (a) first administering to the human subject a SARS-CoV-2 mRNA vaccine formulated in an LNP, and (b) thereafter administering to the human subject a hIL-10R binding agent described herein (e.g., a hIL-10R binding protein (e.g., described herein) or a hIL-10R binding protein encoding mRNA (e.g., described herein)), wherein the hIL-10R binding agent (e.g., the hIL-10R binding protein (e.g., described herein) or the hIL-10R binding protein encoding mRNA (e.g., described herein)) increases the production of nasal IgA in the human subject.

E211. The method of any one of E207-E210, wherein the hIL-10R binding agent (e.g., the hIL-10R binding protein (e.g., described herein) or the hIL-10R binding protein encoding mRNA (e.g., described herein)) is administered intranasally to the human subject.

E212. The method of any one of E207-E211, wherein the mRNA vaccine is administered intramuscularly or subcutaneously to the human subject.

E213. The method of any one of E207-E212, wherein thereafter is from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, or 3 weeks and 1 month.

E214. The method of any one of E207-E213, wherein thereafter is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

E215. The method of any one of E207-E214, wherein thereafter is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days.

E216. The method of any one of E207-E215, wherein the thereafter administering of (b) comprises administering a dose of the hIL-10R binding agent (e.g., the hIL-10R binding protein (e.g., described herein) or the hIL-10R binding protein encoding mRNA (e.g., described herein)) from about 5 μg/kg-160 μg/kg, 10 μg/kg-160 μg/kg, 20 μg/kg-160 μg/kg, 30 μg/kg-160 μg/kg, 40 μg/kg-160 μg/kg, 50 μg/kg-160 μg/kg, 60 μg/kg-160 μg/kg, 70 μg/kg-160 μg/kg, 80 μg/kg-160 μg/kg, 90 μg/kg-160 μg/kg, 100 μg/kg-160 μg/kg, 110 μg/kg-160 μg/kg, 120 μg/kg-160 μg/kg, 130 μg/kg-160 μg/kg, 140 μg/kg-160 μg/kg, or 150 μg/kg-160 μg/kg.

E217. The method of any one of E207-E216, wherein the thereafter administering of (b) comprises administering a dose of the hIL-10R binding agent (e.g., the hIL-10R binding protein (e.g., described herein) or the hIL-10R binding protein encoding mRNA (e.g., described herein)) of about 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, or 1600 μg/kg.

E218. The method of any one of E207-E217, wherein the first administering of (a) comprises intramuscular, subcutaneous, or intranasal administration and the thereafter administering of (b) comprises intramuscular, subcutaneous, or intranasal administration.

E219. The method of any one of E207-E218, wherein the first administering of (a) comprises intramuscular or subcutaneous administration and the thereafter administering of (b) comprises intranasal administration.

E220. A method of preventing, treating, or ameliorating an infection in a subject, the method comprising administering to the subject a hIL-10R binding agent (e.g., a hIL-10R binding protein or a nucleic acid molecule comprising a coding region encoding the hIL-10R binding protein), to thereby prevent, treat, or ameliorate the infection in the subject.

E221. A method of preventing, treating, or ameliorating a disease associated with an infection or preventing, treating, or ameliorating severe disease associated with an infection in a subject, the method comprising administering to the subject a hIL-10R binding agent (e.g., a hIL-10R binding protein or a nucleic acid molecule comprising a coding region encoding the hIL-10R binding protein), to thereby prevent, treat, or ameliorate severe disease associated with the infection in the subject.

E222. A method of enhancing an immunogen-specific immune response in a subject, the method comprising administering to the subject a hIL-10R binding agent (e.g., a hIL-10R binding protein or a nucleic acid molecule comprising a coding region encoding the hIL-10R binding protein), to thereby increase the immunogen specific immune response in the subject.

E223. The method of E222, wherein the duration of the immunogen-specific immune response is increased, magnitude of the immunogen-specific immune response is increased, and/or the nature of the immunogen-specific immune response is changed (e.g., increase in immunogen-specific IgA antibodies).

E224. A method of increasing the level of immunogen-specific mucosal IgA in a subject, the method comprising administering to the subject a hIL-10R binding agent (e.g., a hIL-10R binding protein or a nucleic acid molecule comprising a coding region encoding the hIL-10R binding protein), to thereby increase the level of immunogen specific mucosal IgA in the subject.

E225. The method of E224, wherein the level of immunogen specific mucosal IgA is increased by at least about 1-fold, 10-fold, 100-fold, 1,000-fold, or 10,000-fold.

E226. The method of E224 or E225, wherein the mucosa includes the mucosa of the respiratory tract (e.g., the upper respiratory tract (e.g., nasal mucosa), and/or the lower respiratory tract (e.g., the lungs)).

E227. A method of treating an acute infection (e.g., an acute viral infection, e.g., an acute SARS-CoV-2 infection) in a subject, the method comprising administering to the subject a hIL-10R binding agent (e.g., described herein) (e.g., a hIL-10R binding protein or a nucleic acid molecule comprising a coding region encoding the hIL-10R binding protein), to thereby treat the acute infection.

E228. The method of E227, wherein before the administering, the subject tested positive for the infection.

E229. The method of any one of E227-E228, wherein the infection is a viral infection.

E230. The method of any one of E227-E229, wherein the infection is an acute infection.

E231. The method of any one of E227-E230, wherein the infection is a coronavirus infection (e.g., a SARS-CoV-2 virus infection, a SARS-CoV virus infection, or a MERS-CoV SARS-CoV-2 virus infection).

E232. The method of any one of E227-E231, wherein the infection is a SARS-CoV-2 virus infection.

E233. A method of treating a human subject exposed to an infective agent, comprising administering to the subject an immunogen, or an RNA encoding the immunogen, from the infective agent, in combination with an hIL-10R binding agent.

E234. The method of E233, wherein the infective agent is a virus, e.g., SARS-CoV-2.

E235. The method of any one of E233-234, wherein the immunogen is the same as a vaccine immunogen.

E236. The method of any one of E233-235, wherein the subject has not previously had at least one vaccine dose against the infective agent.

E237. The method of any one of E233-E236, wherein the subject has previously had at least one vaccine dose against the infective agent.

E238. The method of any one of E233-E237, wherein the hIL-10R binding agent is administered after the immunogen, e.g., at least 24 hours to 3 months, e.g., at least 24 hours to 2 months, at least 24 hours to 1 month, at least 24 hours to 3 weeks, at least 24 hours to 2 weeks, at least 24 hours to 1 week, at least 48 hours to 2 months, at least 48 hours to 1 month, at least 48 hours to 3 weeks, at least 48 hours to 2 weeks, at least 48 hours to 1 week, at least 1 week to 2 months, at least 1 week to 1 month, at least 1 week to 3 weeks, at least 1 week to 2 weeks, at least 2 weeks to 2 months, at least 2 weeks to 1 month, at least 2 weeks to 3 weeks, at least 3 weeks to 2 months, or at least 3 weeks to 1 month, after the immunogen.

E239. The method of any one of E233-E238, wherein the subject has an acute infection with the infective agent.

E240. The method of any one of E233-E239, wherein the subject has a post viral syndrome (e.g., long Covid) from a previous acute viral infection.

E241. A method of preventing, ameliorating, or treating an infection (e.g., a viral infection, e.g., a SARS-CoV-2 infection) in a subject, the method comprising administering to the subject a vaccine against the infection, in combination with an hIL-10R binding agent (e.g., a hIL-10R binding protein or a nucleic acid molecule comprising a coding region encoding the hIL-10R binding protein (e.g., described herein)), to thereby prevent the infection.

E242. A method of preventing, ameliorating, or treating severe disease associated with an infection (e.g., a viral infection, e.g., a SARS-CoV-2 infection) in a subject, the method comprising administering to the subject a vaccine, in combination with an hIL-10R binding agent (e.g., a hIL-10R binding protein or a nucleic acid molecule comprising a coding region encoding the hIL-10R binding protein (e.g., described herein)), to thereby prevent severe disease associated with the infection.

E243. The method of any one of E241-E242, wherein before the administering, the subject tested negative for the infection.

E244. The method of any one of E241-E243, wherein the subject has a weakened immune system or weakened immune response (e.g., a weakened immune response to a vaccine).

E245. The method of any one of E241-E244, wherein the subject is immunocompromised or immunosuppressed.

E246. The method of any one of E241-E245, wherein the subject is clinically vulnerable to the infection.

E247. The method of any one of E241-E246, wherein the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age.

E248. The method of any one E241-E247, wherein the subject is at least 50, 55, 60, 65, 70, 75, 80, 85, 90, 100, 110, or 120 years of age; or wherein the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

E249. The method of any one of E241-E248, wherein the subject has been vaccinated (e.g., partially vaccinated or fully vaccinated) against the infection with at least a first dose of an immunogen (e.g., an immunogenic protein or a nucleic acid molecule comprising a coding region encoding the immunogenic protein).

E250. The method of any one of E241-E249, wherein the infection is a viral infection.

E251. The method of any one of E241-E250, wherein the infection is an acute infection.

E252. The method of any one of E241-E251, wherein the infection is a coronavirus infection (e.g., a SARS-CoV-2 virus infection, a SARS-CoV virus infection, or a MERS-CoV SARS-CoV-2 virus infection).

E253. The method of any one of E241-E252, wherein the infection is a SARS-CoV-2 virus infection.

E254. A method of treating or preventing a post viral syndrome, e.g., long COVID, in a subject in need thereof, the method comprising administering to the subject a hIL-10R binding agent (e.g., a hIL-10R binding protein or a nucleic acid molecule comprising a coding region encoding the hIL-10R binding protein (e.g., described herein)), to thereby treat or prevent long COVID in the subject.

E255. The method of E254, wherein the post viral syndrome, e.g., long COVID, results from infection with a SARS-CoV-2 virus, a SARS-CoV virus, or a MERS-CoV SARS-CoV-2 virus).

E256. The method of any one of E254-E255, wherein the subject previously tested positive for SARS-CoV-2 infection but tests negative for SARS-CoV-2 infection prior to administering the hIL-10R binding agent to the subject.

E257. The method of any one of E254-E256, wherein the subject has been vaccinated (e.g., partially vaccinated or fully vaccinated) against a coronavirus (e.g., SARS-CoV-2) with at least a first dose of a coronavirus immunogen (e.g., an immunogenic protein or a nucleic acid molecule comprising a coding region encoding the immunogenic protein), prior to the administration of the hIL-10R binding agent.

E258. A method of promoting the generation of, enhancing the generation of, and/or sustaining the level of plasma cells (e.g., immunogen specific plasma cells (e.g., long lived plasma cells)) in a subject, the method comprising administering to the subject hIL-10R binding agent (e.g., a hIL-10R binding protein or a nucleic acid molecule comprising a coding region encoding the hIL-10R binding protein), to thereby promote the generation of, enhance the generation of, and/or sustain the level of plasma cells in the subject.

E259. The method of E258, wherein first dose of the vaccine regimen comprises an immunogen (e.g., an immunogenic protein or a nucleic acid molecule comprising a coding region encoding the immunogenic protein) from the infective agent (e.g., pathogen) or the tumor.

E260. The method of E258 or E259, wherein the plasma cells specific for the immunogen.

E261. The method of any one of E258-E260, wherein the plasma cells are long lived plasma cells.

E262. The method of any one of E258-E261, wherein the plasma cells (e.g., immunogen specific plasma cells (e.g., long lived plasma cells) are detectable in a sample (e.g., blood sample) obtained from the subject at least 3 months, 6 months, 9 months, 12 months, or longer after the administration of the hIL-10R binding agent.

E263. The method of any one of E207-E262, further comprising administering the immunogen to the subject in combination with the administration of the hIL-10R binding agent (e.g., protein).

E264. The method of any one of E207-E263, wherein the hIL-10R binding agent (e.g., protein) is administered as a booster in prime-boost regimen, wherein the prime portion of the regimen comprises administration of the at least a first dose of the immunogen to the subject; and the boost portion of the regimen comprises the administration of the immunogen and the hIL-10R binding agent (e.g., protein).

E265. The method of any one of E207-E264, wherein the boost portion of the regimen is administered intramuscularly, subcutaneously, or mucosally (e.g., intranasally).

E266. The method of any one of E207-E265, wherein the boost portion of the regimen is administered to the mucosa (e.g., nasal mucosa, gastrointestinal mucosa, urogenital mucosa, oral mucosa, ear mucosa, etc.).

E267. The method of any one of E207-E266, wherein the boost portion of the regimen is administered intranasally.

E268. The method of any one of E207-E267, wherein the prime portion of the regimen is administered intramuscularly, subcutaneously, or mucosally (e.g., intranasally).

E269. The method of any one of E207-E268, wherein the prime portion of the regimen is administered intramuscularly or subcutaneously.

E270. The method of any one of E207-E269, wherein the prime portion of the regimen is administered intramuscularly or subcutaneously; and the boost portion of the regimen is administered intranasally.

E271. The method of any one of E207-E270, wherein the hIL-10R binding agent (e.g., hIL-10R binding protein) is administered to the subject from about 24 hours and 3 months, e.g., 24 hours and 2 months, 24 hours and 1 month, 24 hours and 3 weeks, 24 hours and 2 weeks, 24 hours and 1 week, 48 hours and 2 months, 48 hours and 1 month, 48 hours and 3 weeks, 48 hours and 2 weeks, 48 hours and 1 week, 1 week and 2 months, 1 week and 1 month, 1 week and 3 weeks, 1 week and 2 weeks, 2 weeks and 2 months, 2 weeks and 1 month, 2 weeks and 3 weeks, 3 weeks and 2 months, 3 weeks and 1 month after administration of the at least a first dose of the immunogen to the subject.

E272. The method of any one of E207-E271, wherein the hIL-10R binding agent (e.g., hIL-10R binding protein) is administered to the subject at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen to the subject.

E273. The method of any one of E207-E272, wherein the hIL-10R binding agent (e.g., hIL-10R binding protein) is administered to the subject about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60 days, or 90 days after administration of the at least a first dose of the immunogen to the subject.

E274. The method of any one of E207-E273, wherein the hIL-10R binding agent (e.g., hIL-10R binding protein) is administered intramuscularly, subcutaneously, or mucosally (e.g., intranasally).

E275. The method of any one of E207-E274, wherein the hIL-10R binding agent (e.g., hIL-10R binding protein) is administered mucosally (e.g., intranasally).

E276. The method of any one of E207-E275, wherein the hIL-10R binding agent (e.g., hIL-10R binding protein) is administered intranasally.

E277. The method of any one of E207-E276, wherein the at least a first dose of the immunogen is administered intramuscularly, subcutaneously, or mucosally (e.g., intranasally).

E278. The method of any one of E207-E277, wherein the at least a first dose of the immunogen is administered intramuscularly or subcutaneously.

E279. The method of any one of E207-E278, wherein the hIL-10R binding agent (e.g., hIL-10R binding protein) is administered intranasally and the at least a first dose of the immunogen is administered intramuscularly or subcutaneously.

E280. The method of any one of E207-E279, wherein the hIL-10R binding agent (e.g., hIL-10R binding protein) is administered to the subject at a dose of from about 5 μg/kg-160 μg/kg, 10 μg/kg-160 μg/kg, 20 μg/kg-160 μg/kg, 30 μg/kg-160 μg/kg, 40 μg/kg-160 μg/kg, 50 μg/kg-160 μg/kg, 60 μg/kg-160 μg/kg, 70 μg/kg-160 μg/kg, 80 μg/kg-160 μg/kg, 90 μg/kg-160 μg/kg, 100 μg/kg-160 μg/kg, 110 μg/kg-160 μg/kg, 120 μg/kg-160 μg/kg, 130 μg/kg-160 μg/kg, 140 μg/kg-160 μg/kg, or 150 μg/kg-160 μg/kg.

E281. The method of any one of E207-E280, wherein the hIL-10R binding agent (e.g., hIL-10R binding protein) is administered to the subject at a dose of about 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, or 1600 μg/kg.

E282. The method of any one of E207-E281, wherein the immunogen is a viral immunogen, bacterial immunogen, fungal immunogen, protozoal immunogen, or a tumor associated immunogen.

E283. The method of any one of E207-E282, wherein the immunogen is a viral immunogen.

E284. The method of any one of E207-E283, wherein the immunogen is a respiratory virus immunogen.

E285. The method of any one of E207-E284, wherein the immunogen is a coronavirus immunogen (e.g., a SARS-CoV-2 virus immunogen, a SARS-CoV virus immunogen, a MERS-CoV SARS-CoV-2 virus immunogen), an influenza virus immunogen (e.g., influenza A, influenza B), a respiratory syncytial virus (RSV) immunogen, a rhinovirus immunogen, a parvovirus B19 immunogen, a parainfluenza virus immunogen, or an adenovirus immunogen.

E286. The method of any one of E207-E285, wherein the immunogen is a SARS-CoV-2 spike protein (or an immunogenic fragment or immunogenic variant thereof).

E287. The method of any one of E207-E286, wherein the immunogen is an influenza hemagglutinin immunogen or an influenza neuraminidase immunogen.

E288. The method of any one of E207-E287, wherein the immunogen is an RSV F protein immunogen or an RSV G protein immunogen.

E289. The method of any one of E207-E288, wherein the hIL-10R binding agent (e.g., hIL-10R binding protein) is a hIL-10R agonist.

E290. The method of any one of E207-E289, wherein the hIL-10R binding (e.g., hIL-10R binding protein) is IL-10 (or a functional variant or functional fragment thereof).

E291. The method of any one of E207-E290, wherein the hIL-10R binding (e.g., hIL-10R binding protein) comprises human IL-10 (hIL-10) (or a functional variant or functional fragment thereof).

E292. The method of any one of E207-E291, wherein the hIL-10R binding (e.g., hIL-10R binding protein) comprises a viral IL-10 (vIL-10) (or a functional fragment or functional variant thereof).

E293. The method of any one of E207-E292, wherein the hIL-10R binding (e.g., hIL-10R binding protein) is a polypeptide that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-353 (e.g., SEQ ID NOS: 1-178, SEQ ID NOS: 179-353).

E294. The method of any one of E207-E293, wherein the hIL-10R binding (e.g., hIL-10R binding protein) is a polypeptide that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-3 or 179-181.

E295. The method of any one of E207-E294, wherein the hIL-10R binding (e.g., hIL-10R binding protein) is a polypeptide that comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of 4-178 or 182-353.

E296. The method of any one of E207-E295, wherein the hIL-10R binding (e.g., hIL-10R binding protein) further comprises a heterologous moiety.

E297. The method of E296, wherein the heterologous moiety is operably connected to the hIL-10R binding agent (e.g., hIL-10R binding protein) via a linker.

E298. The method of E296 or E297, wherein the heterologous moiety comprises a heterologous polypeptide (e.g., a half-life extension polypeptide).

E299. The RNA molecule of any one of E296-298, wherein the heterologous polypeptide comprises an immunoglobulin (Ig) Fc region.

E300. The RNA molecule of E299, wherein the Ig Fc region comprises at least a portion of a hinge region, a CH2 region, and a CH3 region.

E301. The RNA molecule of any one of E299-E300, wherein the Ig Fc region comprises a hinge region, a CH2 region, and a CH3 region.

E302. The RNA molecule of any one of E299-E301, wherein the heterologous polypeptide comprises a human immunoglobulin (hIg) Fc region.

E303. The method of E302, wherein the hIg is a human IgG (hIgG).

E304. The method of E302, wherein the hIgG is hIgG1 or hIgG4.

E305. The method of any one of E299-E301, wherein the heterologous polypeptide comprises a murine immunoglobulin (mIg) Fc region.

E306. The method of E305, wherein the mIg is a mIgG1.

E307. The method of E305, wherein the mIg is a mIgG2a.

E308. The method of any one of E299-E307, wherein the Ig Fc region comprises one or more amino acid substitutions relative to a reference hIg Fc region that reduces or abolishes one or more of the following effector functions relative to the reference hIg Fc region: antibody dependent cell mediated cytotoxicity (ADCC), complement dependent cytotoxicity (CDC), and/or affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E309. The method of any one of E299-E308, wherein the Ig Fc region does not substantially mediate ADCC, does not substantially mediate CDC, and/or does not bind to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

E310. The method of any one of E207-E309, wherein the hIL-10R binding agent (e.g., hIL-10R binding protein) comprises a protein or a nucleic acid molecule comprising a coding region encoding the hIL-10R binding agent (e.g., hIL-10R binding protein).

E311. The method of E310, wherein the hIL-10R binding agent comprises a nucleic acid molecule encoding the hIL-10R binding agent (e.g., hIL-10R binding protein).

E312. The method of E311, wherein the nucleic acid molecule is a DNA molecule or an RNA molecule.

E313. The method of E311-E312, wherein the nucleic acid molecule is an RNA molecule.

E314. The method of E313, wherein the RNA molecule is an mRNA molecule or a circular RNA molecule.

E315. The method of E313-314, wherein the RNA molecule is the RNA molecule of any one of E141-195.

E316. The method of any one of E207-E315, wherein the nucleotide sequence of the nucleic acid molecule comprises at least one modified nucleotide.

E317. The method of any one of E207-E316, wherein the nucleotide sequence of the nucleic acid molecule comprises N1-methyl-pseudouridine, cytosine, adenine, and guanine.

E318. The method of any one of E207-E317, wherein the nucleic acid molecule comprises a heterologous 5′-untranslated region (UTR), 3′-UTR, or both a 5′-UTR and 3′-UTR.

E319. The method of any one of E207-E318, wherein the nucleic acid molecule comprises a poly(A) sequence.

E320. The method of any one of E207-E319, wherein the nucleic acid molecule comprises a 5′cap structure.

E321. The method of any one of E207-E320, wherein the nucleotide sequence of the nucleic acid molecule is codon optimized.

E322. The method of any one of E207-E321, wherein the nucleic acid molecule further comprises a coding region encoding the immunogen.

E323. The method of any one of E207-E322, wherein the nucleic acid molecule is comprised within a vector.

E324. The method of E232, wherein the hIL-10R binding agent (e.g., hIL-10R binding protein) comprises a protein.

E325. The method of any one of E207-E324, further comprising administering an IGIP (e.g., hIGIP) protein (or a nucleic acid molecule comprising a coding region encoding the IGIP (e.g., hIGIP) protein) to the subject.

E326. The method of E325, comprising administering an IGIP (e.g., hIGIP) protein to the subject.

E327. The method of E325 or E326, wherein the IGIP (e.g., hIGIP) protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572 or a protein set forth in Table 13.

E328. The method of E325, comprising administering a nucleic acid molecule comprising a coding region encoding the IGIP (e.g., hIGIP) protein) to the subject.

E329. The method of E328, wherein the encoded IGIP (e.g., hIGIP) protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 570-572 or a protein set forth in Table 13.

E330. The method of any one of E325-E329, wherein the IGIP (e.g., hIGIP) protein) (or the nucleic acid molecule comprising a coding region encoding the IGIP (e.g., hIGIP) protein) is administered to the subject prior to, concurrent with, and/or after administration of the hIL-10R binding (e.g., hIL-10R binding protein) (or the nucleic acid molecule encoding the same to the subject.

E331. The method of any one of E325-E330, wherein the IGIP (e.g., hIGIP) protein) (or the nucleic acid molecule comprising a coding region encoding the IGIP (e.g., hIGIP) protein) is administered to the subject prior to, concurrent with, and/or after administration of the immunogen to the subject.

E332. The method of any one of E307-E331, wherein the hIL-10R binding agent (e.g., hIL-10R binding protein) is comprised within a carrier.

E333. The method of any one E332, wherein the carrier is a lipid nanoparticle (LNP), liposome, lipoplex, or nanoliposome.

E334. The method of any one of E333, wherein the carrier is an LNP.

E335. The method of any one of E334, wherein the LNP comprises a cationic lipid, a neutral lipid, a cholesterol, and/or a PEG lipid.

E336. The method of any one of E334 or E335, wherein the LNP has a mean particle size of between 80 nm and 160 nm.

E337. The method of any one of E207-E336, wherein the hIL-10R binding agent is comprised within a composition.

E338. The method of E337, wherein the composition comprises the combination regimen of any one of E1-E27 or composition of any one of E28-140.

E339. A method of any one of E338, wherein the composition is a pharmaceutical composition comprising a pharmaceutically acceptable excipient.

E340. The method of any one of E337-E338, wherein the composition is formulated for mucosal delivery.

E341. The method of any one of E337-339, wherein the composition is formulated for intranasal delivery.

E342. The method of any one of E207-E341, wherein the subject has been vaccinated against the infection with at least a first dose of an immunogen.

E343. The method of any one of E207-E342, comprising administering to the subject an immunogen (e.g., immunogenic protein (or an immunogenic fragment and/or immunogenic variant thereof) or a nucleic acid molecule comprising a coding region encoding an immunogenic protein (or an immunogenic fragment and/or immunogenic variant thereof), in combination with the hIL-10R binding protein (or the functional fragment and/or functional variant thereof) or the nucleic acid molecule comprising a coding region encoding a hIL-10R binding protein (or the functional fragment and/or functional variant thereof)).

### 6. EXAMPLES

Table of Contents

| | |
|---|---|
| 6.1 | Example 1. hIL-10R Binding Fusion Protein Expression and Characterization. |
| 6.2 | Example 2. In vitro Stimulation of B Cell Antibody Production. |
| 6.3 | Example 3. In vitro B Cell Stimulation and Quantification of Antigen-Specific Antibody |
| | Production. |
| 6.4 | Example 4. In vitro B Cell Stimulation and Analysis of B Cell Populations. |
| 6.5 | Example 5. Differential hIL-10R Subunit Expression and Binding by hIL-10R Binding |
| | Agents Described Herein. |
| 6.6 | Example 6. In Vitro Enhancement of B Cell Responses Through the Promotion of T Cell |
| | Help. |
| 6.7 | Example 7. Suppression of Immune Cell Functions Associated with Vaccine |
| | Reactogenicity. |

### 6.1 Example 1. hIL-10R Binding Fusion Protein Expression and Characterization

A set of hIL-10R binding fusion proteins (hIL-10R BFPs) containing from N- to C-terminus: a hIL-2 signal sequence, an effector function reduced hIgG4 Fc region, a peptide linker, and a hIL-10R binding protein (hIL-10R BP) described herein, were generated using standard methods known in the art. Briefly, a DNA polynucleotide encoding each of the hIL-10R BFPs was synthesized and inserted into an expression plasmid. Expi293 cells (Thermo Fisher #A14527) were transfected using the Expi293 expression kit (Thermo Fisher #A14635) according to the manufacturer's protocol. Briefly, Expi293 cells were grown in suspension at 37° C., 8% CO₂ in Expi293 growth medium (Thermo Fisher #A1435101). The cells were counted using a hemocytometer to ensure a density of 2.5-3 million cells per mL, and a viability above 95%, prior to transfection. Transfections were performed in 2.5 ml of cell containing medium (7.5-9 million cells per reaction). 1 μg/ml of plasmid DNA was pre-incubated with Opti-MEM for 5 minutes at room temperature (RT) and ExpiFectamine was pre-incubated with Opti-MEM for 5 minutes at RT. The plasmid mixture was subsequently mixed with the ExpiFectamine mixture and incubated for 10-20 minutes at RT. After incubation, the mixture was added to the Expi293 cells and incubated overnight. On day 1 post-transfection, ExpiFectamine Enhancer 1 and ExpiFectamine Enhancer 2 were added to the cell culture. On day 3 post-transfection, the supernatant was removed and maintained at −20° C., and the cells were discarded. The amino acid sequence of the immature and mature form of each of the generated hIL-10R BFPs (hIL-10R BFPs-1-2 and 4-14) is set forth in Table 9. Each of the generated hIL-10R BFPs was determined to engage the hIL-10R. An Fc-GFP control fusion protein was also generated with the amino acid sequence set forth in Table 9.

TABLE 9
Amino Acid Sequence of hIL-10R BFPs and Control

| | | SEQ ID |
|---|---|---|
| Description | Amino Acid Sequence | NO |
| hIL-10R BFP-1 | MYRMQLLSCIALSLALVINSAESKYGPPCPPCPAPEAAGGPSVELEPPK | 412 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMHSSALLCCLVLLTGVRASPGQGTQSE | |
| | NSCTHEPGNLPNMLRDLRDAFSRVKTFFQMKDQLDNLLLKESLLEDFKG | |
| | YLGCQALSEMIQFYLEEVMPQAENQDPDIKAHVNSLGENLKTLRLRLRR | |
| | CHRFLPCENKSKAVEQVKNAFNKLQEKGIYKAMSEFDIFINYIEAYMTM | |
| | KIRN | |
| hIL-10R BFP-1 | AESKYGPPCPPCPAPEAAGGPSVELFPPKPKDTLMISRTPEVTCVVVDV | 413 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMHSSALLCCLVLLTGVRASPGQGTQSENSCTHFPGNLPNMLRDLRDA | |
| | FSRVKTFFQMKDQLDNLLLKESLLEDFKGYLGCQALSEMIQFYLEEVMP | |
| | QAENQDPDIKAHVNSLGENLKTLRLRLRRCHRFLPCENKSKAVEQVKNA | |
| | FNKLQEKGIYKAMSEFDIFINYIEAYMTMKIRN | |
| hIL-10R BP-1 | AESKYGPPCPPCPAPEAAGGPSVELFPPKPKDTLMISRTPEVTCVVVDV | 522 |
| without hIL-2 | SQEDPEVQFNWYVDGVEVHNAKTKPREEQFNSTYRVVSVLTVLHQDWLN | |
| signal peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSSPGQGTQSENSCTHFPGNLPNMLRDLRDAFSRVKTFFQMKDQLDNLL | |
| | LKESLLEDFKGYLGCQALSEMIQFYLEEVMPQAENQDPDIKAHVNSLGE | |
| | NLKTLRLRLRRCHRFLPCENKSKAVEQVKNAFNKLQEKGIYKAMSEFDI | |
| | FINYIEAYMTMKIRN | |
| hIL-10R BFP-2 | MYRMQLLSCIALSLALVINSAESKYGPPCPPCPAPEAAGGPSVELEPPK | 414 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMHSSALLCCLVLLTGVRASPGQGTQSE | |
| | NSCTHFPGYLPNMLRDLRDAFSRVKTFFQMKDQLDNLLLKESLLEDEKG | |
| | YLGCQALSEMIQFYLEEVMPQAENQDPDIKAHVQSLGENLKDLRLWLRR | |
| | CHRFLPCENKSKAVEQVKNAFNKLQEKGIYKAMSEFDIFINYIEAYMTM | |
| | KIRN | |
| hIL-10R BFP-2 | AESKYGPPCPPCPAPEAAGGPSVFLFPPKPKDTLMISRTPEVTCVVVDV | 415 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMHSSALLCCLVLLTGVRASPGQGTQSENSCTHFPGYLPNMLRDLRDA | |
| | FSRVKTFFQMKDQLDNLLLKESLLEDFKGYLGCQALSEMIQFYLEEVMP | |
| | QAENQDPDIKAHVQSLGENLKDLRLWLRRCHRELPCENKSKAVEQVKNA | |
| | FNKLQEKGIYKAMSEFDIFINYIEAYMTMKIRN | |
| hIL-10R BP-4 | MYRMQLLSCIALSLALVTNSAESKYGPPCPPCPAPEAAGGPSVELFPPK | 420 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMGKRAFVVSVAMALLGIYVITNTVNAR | |
| | HCMFGDSLRNSPDMKNMLQDLRGGYSGSGIKRTFQGKDTLDSMLLTQSL | |
| | LDDFKGYLGCQALSEMIQFYLEEVMPQAENHGPTDSVKQLGEKLHTLNQ | |
| | KFGECPRWFPCYYNTTPAVENVKSVESKLQERGVYKAMSEFDIFINYIE | |
| | TYTTMK | |
| hIL-10R BP-4 | AESKYGPPCPPCPAPEAAGGPSVFLFPPKPKDTLMISRTPEVTCVVVDV | 430 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVESCSVMHEALHNHYTQKSLSLSLGGGGGGGGSGGGGSGGG | |
| | GSMGKRAFVVSVAMALLGIYVITNTVNARHCMFGDSLRNSPDMKNMLQD | |
| | LRGGYSGSGIKRTFQGKDTLDSMLLTQSLLDDFKGYLGCQALSEMIQFY | |
| | LEEVMPQAENHGPTDSVKQLGEKLHTLNQKFGECPRWFPCYYNTTPAVE | |
| | NVKSVFSKLQERGVYKAMSEFDIFINYIETYTTMK | |
| hIL-10R BP-5 | MYRMQLLSCIALSLALVTNSAESKYGPPCPPCPAPEAAGGPSVELFPPK | 421 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMARRLTVASCGSVSLLAAFAAVLLIGC | |
| | QLESGEALPLGSRSADSRSVDGQRVPAPQNNYPGLLRDLRLGYEGFKQK | |
| | VTDSHPDETLLGSSRLAGDLKGPLRCQALSEMIQFLLQVVLPDAENSRQ | |
| | DLRSQFSTLGDRITGLRQQLRRDPTVFPCESRSDGVSDLRSAYTRLGST | |
| | GAEKVLSEFDIFINYIEAYVTSV | |
| hIL-10R BP-5 | AESKYGPPCPPCPAPEAAGGPSVFLFPPKPKDTLMISRTPEVTCVVVDV | 431 |
| without signal | SQEDPEVQFNWYVDGVEVHNAKTKPREEQENSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMARRLTVASCGSVSLLAAFAAVLLIGCQLESGEALPLGSRSADSRSV | |
| | DGQRVPAPQNNYPGLLRDLRLGYEGFKQKVTDSHPDETLLGSSRLAGDL | |
| | KGPLRCQALSEMIQFLLQVVLPDAENSRQDLRSQFSTLGDRITGLRQQL | |
| | RRDPTVFPCESRSDGVSDLRSAYTRLGSTGAEKVLSEFDIFINYIEAYV | |
| | TSV | |
| hIL-10R BP-6 | MYRMQLLSCIALSLALVTNSAESKYGPPCPPCPAPEAAGGPSVELFPPK | 422 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMSNNKILVCAVIILTYTLYTDAYCVEY | |
| | AESDEDRQQCSSSSNFPASLPHMLRELRAAFGKVKTFFQMKDQLNSMLL | |
| | TQSLLDDFKGYLGCQALSEMIQFYLEEVMPQAENHGPDIKEHVNSLGEK | |
| | LKTLRLRLRRCHRFLPCENKSKAVEQVKRVENMLQERGVYKAMSEFDIF | |
| | INYIESYMTTKM | |
| hIL-10R BP-6 | AESKYGPPCPPCPAPEAAGGPSVFLFPPKPKDTLMISRTPEVTCVVVDV | 432 |
| without signal | SQEDPEVQFNWYVDGVEVHNAKTKPREEQENSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMSNNKILVCAVIILTYTLYTDAYCVEYAESDEDRQQCSSSSNFPASL | |
| | PHMLRELRAAFGKVKTFFQMKDQLNSMLLTQSLLDDEKGYLGCQALSEM | |
| | IQFYLEEVMPQAENHGPDIKEHVNSLGEKLKTLRLRLRRCHRFLPCENK | |
| | SKAVEQVKRVENMLQERGVYKAMSEFDIFINYIESYMTTKM | |
| hIL-10R BP-7 | MYRMQLLSCIALSLALVINSAESKYGPPCPPCPAPEAAGGPSVELFPPK | 423 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMANVVYVVLVISIMMANIHVSKTYCTS | |
| | CSHHQCTEDENQKQDCEDANHSLPHMLRELRAAFGKVKTFFQMKDQLHS | |
| | LLLTQSLLDDFKGYLGCQALSEMIQFYLEEVMPQAENHGPEEHDNSLSE | |
| | HGPDVKEHVNSLGEKLKTLRLRLRRCHRFLPCENKSKAVEKVKRVESEL | |
| | QERGVYKAMSEFDIFINYIETYMTT | |
| hIL-10R BP-7 | AESKYGPPCPPCPAPEAAGGPSVFLFPPKPKDTLMISRTPEVTCVVVDV | 433 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMANVVYVVLVISIMMANIHVSKTYCTSCSHHQCTEDENQKQDCEDAN | |
| | HSLPHMLRELRAAFGKVKTFFQMKDQLHSLLLTQSLLDDEKGYLGCQAL | |
| | SEMIQFYLEEVMPQAENHGPEEHDNSLSEHGPDVKEHVNSLGEKLKTLR | |
| | LRLRRCHRFLPCENKSKAVEKVKRVESELQERGVYKAMSEFDIFINYIE | |
| | TYMTT | |
| hIL-10R BP-8 | MYRMQLLSCIALSLALVTNSAESKYGPPCPPCPAPEAAGGPSVELFPPK | 424 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMQGLQLLRGLLCCGVFAAASSRSPKNK | |
| | PSIDCNPQTGDFVNMLKSMRQDYSRIRDTLHDRDKLHSSLLTGALLDEM | |
| | MGYSGCRTTLLLMEHYLDTWYPAAYRHHLYDNQTLVVVDRMGSTLVALL | |
| | KAMVQCPMLACGAPSPAMDKMLQQEAKMKKYTGVYKGISETDLLLGYLE | |
| | LYMMKFKR | |
| hIL-10R BP-8 | AESKYGPPCPPCPAPEAAGGPSVELFPPKPKDTLMISRTPEVTCVVVDV | 434 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVESCSVMHEALHNHYTQKSLSLSLGGGGGGGGSGGGGSGGG | |
| | GSMQGLQLLRGLLCCGVFAAASSRSPKNKPSIDCNPQTGDFVNMLKSMR | |
| | QDYSRIRDTLHDRDKLHSSLLTGALLDEMMGYSGCRTTLLLMEHYLDTW | |
| | YPAAYRHHLYDNQTLVVVDRMGSTLVALLKAMVQCPMLACGAPSPAMDK | |
| | MLQQEAKMKKYTGVYKGISETDLLLGYLELYMMKEKR | |
| hIL-10R BP-9 | MYRMQLLSCIALSLALVTNSAESKYGPPCPPCPAPEAAGGPSVELFPPK | 425 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMRRRRSFGIVVSGAIRTLLMVAVVAVS | |
| | VRGHEHKVPPACDPVHGNLAGIFKELRAIYASIREALQKKDTVYYTSLF | |
| | NDRVLQEMLSPMGCRVTNELMEHYLDGVLPRAAHFDYDNSTLNGLHAFT | |
| | SSMQALYQHMLKCPALACTGKTPAWMYFLEVEHKLNPWRGTAKAAAEAD | |
| | LLLNYLETFLLQF | |
| hIL-10R BP-9 | AESKYGPPCPPCPAPEAAGGPSVFLFPPKPKDTLMISRTPEVTCVVVDV | 435 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMRRRRSFGIVVSGAIRTLLMVAVVAVSVRGHEHKVPPACDPVHGNLA | |
| | GIFKELRAIYASIREALQKKDTVYYTSLENDRVLQEMLSPMGCRVTNEL | |
| | MEHYLDGVLPRAAHFDYDNSTLNGLHAFTSSMQALYQHMLKCPALACTG | |
| | KTPAWMYFLEVEHKLNPWRGTAKAAAEADLLLNYLETFLLQF | |
| hIL-10R BFP- | MYRMQLLSCIALSLALVTNSAESKYGPPCPPCPAPEAAGGPSVELEPPK | 416 |
| 10 | PKDTLMISRTPEVTCVVVDVSQEDPEVQFNWYVDGVEVHNAKTKPREEQ | |
| with signal | FNSTYRVVSVLTVLHQDWLNGKEYKCKVSNKGLPSSIEKTISKAKGQPR | |
| peptide | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMGSRRLSRCSFATAVCLVAIVAAVAAK | |
| | GRDSKPSPACDPMHGALAGIFKELRTTYRSVREALQTKDTVYYVSLFHE | |
| | QLLQEMLSPVGCRVTNELMQHYLDGVLPRAFHCGYDNATLNALHALSSS | |
| | LSTLYQHMLKCPALACTGQTPAWTQFLDTEHKLDPWKGTVKATAEMDLL | |
| | LNYLETFLLQS | |
| hIL-10R BFP- | AESKYGPPCPPCPAPEAAGGPSVFLFPPKPKDTLMISRTPEVTCVVVDV | 417 |
| 10 | SQEDPEVQFNWYVDGVEVHNAKTKPREEQFNSTYRVVSVLTVLHQDWLN | |
| without signal | GKEYKCKVSNKGLPSSIEKTISKAKGQPREPQVYTLPPSQEEMTKNQVS | |
| peptide | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMGSRRLSRCSFATAVCLVAIVAAVAAKGRDSKPSPACDPMHGALAGI | |
| | FKELRTTYRSVREALQTKDTVYYVSLFHEQLLQEMLSPVGCRVTNELMQ | |
| | HYLDGVLPRAFHCGYDNATLNALHALSSSLSTLYQHMLKCPALACTGQT | |
| | PAWTQFLDTEHKLDPWKGTVKATAEMDLLLNYLETFLLQS | |
| hIL-10R BP-10 | AESKYGPPCPPCPAPEAAGGPSVFLFPPKPKDTLMISRTPEVTCVVVDV | 534 |
| without hIL-2 | SQEDPEVQFNWYVDGVEVHNAKTKPREEQFNSTYRVVSVLTVLHQDWLN | |
| signal peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSKGRDSKPSPACDPMHGALAGIFKELRTTYRSVREALQTKDTVYYVSL | |
| | FHEQLLQEMLSPVGCRVTNELMQHYLDGVLPRAFHCGYDNATLNALHAL | |
| | SSSLSTLYQHMLKCPALACTGQTPAWTQFLDTEHKLDPWKGTVKATAEM | |
| | DLLLNYLETELLQS | |
| hIL-10R BP-11 | MYRMQLLSCIALSLALVINSAESKYGPPCPPCPAPEAAGGPSVELEPPK | 426 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMLSVMVSSSLVLIVFELGASEEAKPAT | |
| | TTIKNTKPQCRPEDYATRLQDLRVTFHRVKPTLQREDDYSVWLDGTVVK | |
| | GCWGCSVMDWLLRRYLEIVFPAGDHVYPGLKTELHSMRSTLESIYKDMR | |
| | QCPLLGCGDKSVISRLSQEAERKSDNGTRKGLSELDTLESRLEEYLHSR | |
| | K | |
| hIL-10R BP-11 | AESKYGPPCPPCPAPEAAGGPSVFLFPPKPKDTLMISRTPEVTCVVVDV | 436 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMLSVMVSSSLVLIVFFLGASEEAKPATTTIKNTKPQCRPEDYATRLQ | |
| | DLRVTFHRVKPTLQREDDYSVWLDGTVVKGCWGCSVMDWLLRRYLEIVE | |
| | PAGDHVYPGLKTELHSMRSTLESIYKDMRQCPLLGCGDKSVISRLSQEA | |
| | ERKSDNGTRKGLSELDTLFSRLEEYLHSRK | |
| hIL-10R BP-12 | MYRMQLLSCIALSLALVTNSAESKYGPPCPPCPAPEAAGGPSVFLFPPK | 427 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMALAHQLPVWIFSIWILYFTLPLSEER | |
| | VLPLRGNCKLLLQDTVIPNLLYSMRSIFQDIKPYFQGKDSLNNLLLSGQ | |
| | LLEDLQSPIGCDALSEMIQFYLEEVMPQAEIHHPKHKNSVMQLGETLHT | |
| | LISQLQECTALFPCKHKSLGAQKIKEEVSKLGQYGIIKAVAEFDIFINY | |
| | MESYFGVK | |
| hIL-10R BP-12 | AESKYGPPCPPCPAPEAAGGPSVFLFPPKPKDTLMISRTPEVTCVVVDV | 437 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMALAHQLPVWIFSIWILYFTLPLSEERVLPLRGNCKLLLQDTVIPNL | |
| | LYSMRSIFQDIKPYFQGKDSLNNLLLSGQLLEDLQSPIGCDALSEMIQF | |
| | YLEEVMPQAEIHHPKHKNSVMQLGETLHTLISQLQECTALFPCKHKSLG | |
| | AQKIKEEVSKLGQYGIIKAVAEFDIFINYMESYFGVK | |
| hIL-10R BP-13 | MYRMQLLSCIALSLALVINSAESKYGPPCPPCPAPEAAGGPSVELFPPK | 428 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMRRRRRSFGIIVAGAIGTLLMMAVVVL | |
| | SAHDHEHKEVPPACDPVHGNLAGIFKELRATYASIREGLQKKDTVYYTS | |
| | LENDRVLHEMLSPMGCRVTNELMEHYLDGVLPRASHLDYDNSTLNGLHV | |
| | FASSMQALYQHMLKCPALACTGKTPAWMYFLEVEHKLNPWRGTAKAAAE | |
| | ADLLLNYLETFLLQF | |
| hIL-10R BP-13 | AESKYGPPCPPCPAPEAAGGPSVELFPPKPKDTLMISRTPEVTCVVVDV | 438 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMRRRRRSFGIIVAGAIGTLLMMAVVVLSAHDHEHKEVPPACDPVHGN | |
| | LAGIFKELRATYASIREGLQKKDTVYYTSLENDRVLHEMLSPMGCRVTN | |
| | ELMEHYLDGVLPRASHLDYDNSTLNGLHVFASSMQALYQHMLKCPALAC | |
| | TGKTPAWMYFLEVEHKLNPWRGTAKAAAEADLLLNYLETELLQF | |
| hIL-10R BP-14 | MYRMQLLSCIALSLALVTNSAESKYGPPCPPCPAPEAAGGPSVELFPPK | 429 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVESCSVMHEALHNHYTQKSLS | |
| | LSLGGGGGGGGSGGGGSGGGGSMRRRRSFGIVVAGAIGTLLMMAVVVES | |
| | AHEHKEVPPACDPVHGNLAGIFKELRATYASIREGLQKKDTVYYTSLEN | |
| | DRVLQEMLSPMGCRVTNELMEHYLDGVLPRALHLDYDNSTLNGLHAFAS | |
| | SMQALYQHMLKCPALACTGKTPAWMYFLEVEHKLNPWRGTAKAAAEADL | |
| | LLNYLETFLLQF | |
| hIL-10R BP-14 | AESKYGPPCPPCPAPEAAGGPSVELFPPKPKDTLMISRTPEVTCVVVDV | 439 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMRRRRSFGIVVAGAIGTLLMMAVVVESAHEHKEVPPACDPVHGNLAG | |
| | IFKELRATYASIREGLQKKDTVYYTSLENDRVLQEMLSPMGCRVTNELM | |
| | EHYLDGVLPRALHLDYDNSTLNGLHAFASSMQALYQHMLKCPALACTGK | |
| | TPAWMYFLEVEHKLNPWRGTAKAAAEADLLLNYLETELLQF | |
| Fc-GFP | MYRMQLLSCIALSLALVTNSAESKYGPPCPPCPAPEAAGGPSVELEPPK | 418 |
| with signal | PKDTLMISRTPEVTCVVVDVSQEDPEVQFNWYVDGVEVHNAKTKPREEQ | |
| peptide | FNSTYRVVSVLTVLHQDWLNGKEYKCKVSNKGLPSSIEKTISKAKGQPR | |
| | EPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLS | |
| | LSLGGGGGGGGSGGGGSGGGGSMRKGEELFTGVVPILVELDGDVNGHKE | |
| | SVSGEGEGDATNGKLTLKFICTTGKLPVPWPTLVTTLTYGVQCFARYPD | |
| | HMKQHDFFKSAMPEGYVQERTISFKDDGTYKTRAEVKFEGDTLVNRIEL | |
| | KGIDFKEDGNILGHKLEYNENSHNVYITADKQKNGIKANFKIRHNVEDG | |
| | SVQLADHYQQNTPIGDGPVLLPDNHYLSTQSALSKDPNEKRDHMVLLEF | |
| | VTAAGITHGMDELYKRPAANDENYAASV | |
| Fc-GFP | AESKYGPPCPPCPAPEAAGGPSVFLFPPKPKDTLMISRTPEVTCVVVDV | 419 |
| without signal | SQEDPEVQFNWYVDGVEVHNAKTKPREEQFNSTYRVVSVLTVLHQDWLN | |
| peptide | GKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVD | |
| | KSRWQEGNVFSCSVMHEALHNHYTQKSLSLSLGGGGGGGGSGGGGSGGG | |
| | GSMRKGEELFTGVVPILVELDGDVNGHKESVSGEGEGDATNGKLTLKFI | |
| | CTTGKLPVPWPTLVTTLTYGVQCFARYPDHMKQHDFFKSAMPEGYVQER | |
| | TISFKDDGTYKTRAEVKFEGDTLVNRIELKGIDFKEDGNILGHKLEYNE | |
| | NSHNVYITADKQKNGIKANFKIRHNVEDGSVQLADHYQQNTPIGDGPVL | |
| | LPDNHYLSTQSALSKDPNEKRDHMVLLEFVTAAGITHGMDELYKRPAAN | |
| | DENYAASV | |

### 6.2 Example 2. In vitro Stimulation of B Cell Antibody Production

The ability of each of: hIL-10R BFP-1 (Fc-hIL-10) (SEQ ID NO: 413); hIL-10R BFP-2 (Fe fusion of high affinity version of IL-10; Saxton et al. Science. 2021 Mar. 19: 371(6535), the entire contents of which is incorporated by reference herein for all purposes) (SEQ ID NO: 415); and hIL-10R BFP-10 (SEQ ID NO: 417) (see Table 9) was assessed for its ability to stimulate antibody production from B cells, including each of IgM, IgG, and IgA antibody isotypes. Briefly, human donor peripheral blood mononuclear cells (PBMCs) were thawed by standard methods and subsequently plated in 96 well plates at a concentration of 200,000 cells/well in IMDM media with 10% heat-inactivated fetal bovine serum (FBS). The cells were stimulated with a dose titration (1-1000 pM) of the three IL-10R BFPs described above or a control (Fc-GFP)+/−0.1 μg/mL CD40 ligand (CD40L) (Fisher Scientific #NC9975949) and +/−50 ng/mL human IL-4 (StemCell #78147.1), and incubated at 37° C., 5% CO₂ for 12 days.

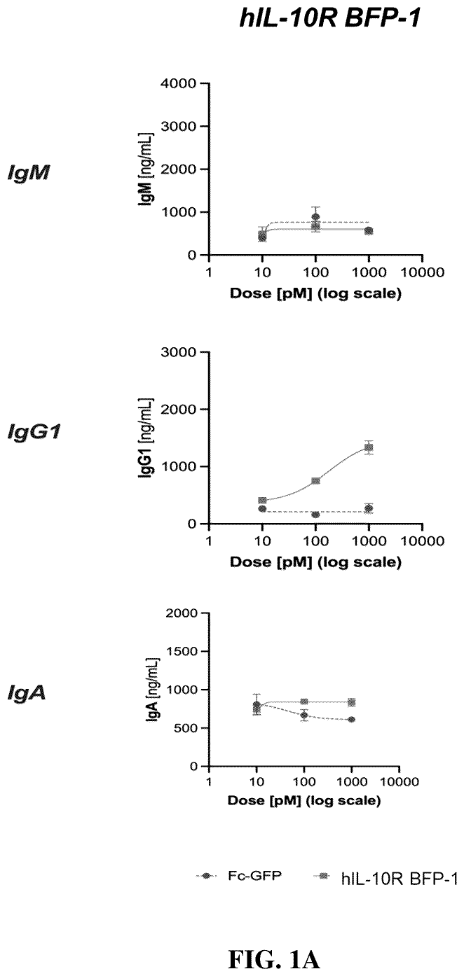

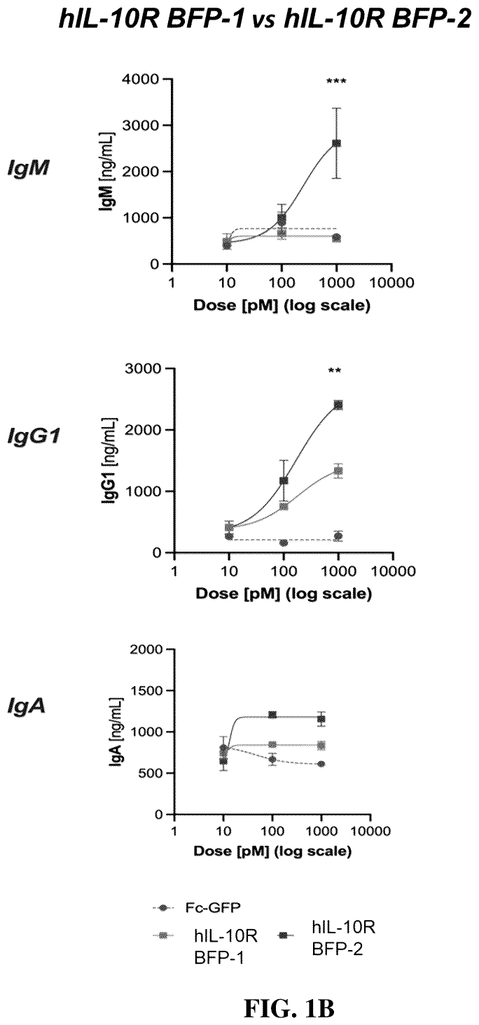

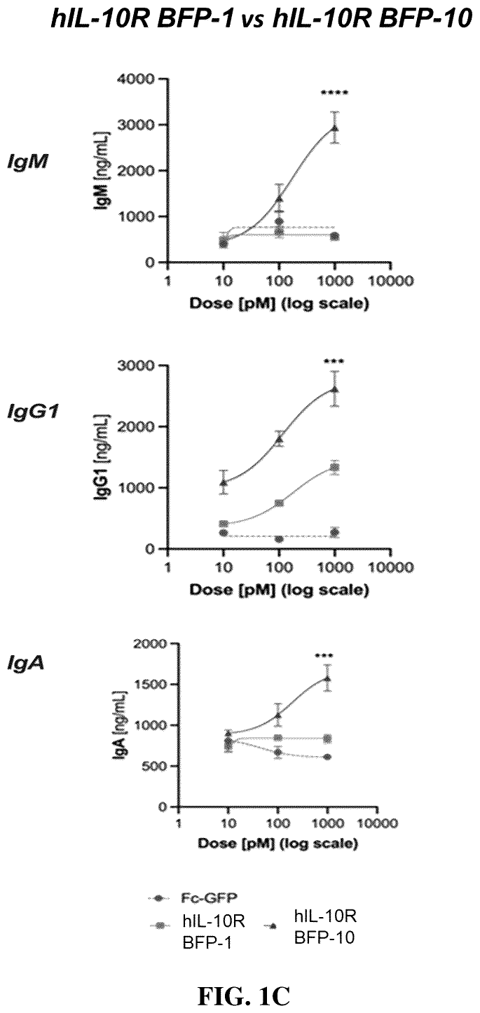

After 12 days, the cells were centrifuged for 1 minute at 500 g and the supernatant was removed for Ig flow cytometry analysis. Antibody concentrations (IgG1, IgG2, IgG3, IgG4, IgA, IgE, IgM, & IgD isotypes) from the human PBMC stimulated supernatants were determined using the LegendPlex human immunoglobulin isotyping panel (BioLegend #740638) per the manufacturer's protocol. Briefly, supernatants, capture beads, and assay buffer were added to 96-well plates and incubated at room temperature while shaking for 2 hours. Plates were washed and detection antibodies added. The plates were then incubated at room temperature while shaking for 1 hour. Detection reagent streptavidin, r-phycoerythrin conjugate (SA-PE) was added and incubated at room temperature while shaking for 30 minutes. The capture beads were spun down and resuspended in wash buffer for flow cytometry analysis. Analysis was performed using a Cytoplex flow cytometer and Qognit software. As shown in FIGS. 1A-1C, all of the hIL-10R BFPs assessed (hIL-10R BFP-1 (FIG. 1A), hIL-10R BFP-2 (FIG. 1B), and hIL-10R BFP-10 (FIG. 1C)) induced a dose dependent increase in the production of IgG and IgA antibodies.

### 6.3 Example 3. In vitro B Cell Stimulation and Quantification of Antigen-Specific Antibody Production

The ability of each of: hIL-10R BFP-1 (Fc-hIL-10) (SEQ ID NO: 413); hIL-10R BFP-2 (SEQ ID NO: 415); and hIL-10R BFP-10 (SEQ ID NO: 417) (see Table 9), was assessed for its ability to stimulate antigen-specific antibody production from B cells. More specifically, a SARS-Cov-2 Ig Fc fusion protein (SEQ ID NO: 469) (see Table 10) was utilized to assess the ability of each of the hIL-10R agonists to induce production of anti-SARS-CoV-2 antibodies (IgG and IgA) from human PBMCs obtained from (1) a donor known to have been previously administered a SARS-Cov-2 vaccine (Vaccinated Donor) or (2) a donor whose prior SARS-CoV-2 vaccination status was unknown (Unknown Donor).

TABLE 10
Amino Acid Sequence of SARS-CoV-2 Spike Fc Fusion Protein

| | | SEQ ID |
|---|---|---|
| Description | Amino Acid Sequence | NO |
| SARS-CoV-2 | MYRMQLLSCIALSLALVINSAESKYGPPCPPCPAPEAAGGPSVFLFPPK | 468 |
| Spike Fc | PKDTLMISRTPEVTCVVVDVSQEDPEVQFNWYVDGVEVHNAKTKPREEQ | |
| Fusion Protein | FNSTYRVVSVLTVLHQDWLNGKEYKCKVSNKGLPSSIEKTISKAKGQPR | |
| with signal | EPQVYTLPPSQEEMTKNQVSLTCLVKGFYPSDIAVEWESNGQPENNYKT | |
| peptide | TPPVLDSDGSFFLYSRLTVDKSRWQEGNVESCSVMHEALHNHYTQKSLS | |
| | LSLGGGGGGGGSGGGGSGGGGSQCVNLTTRTQLPPAYTNSFTRGVYYPD | |
| | KVFRSSVLHSTQDLFLPFFSNVTWFHAIHVSGTNGTKREDNPVLPENDG | |
| | VYFASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKQGNEK | |
| | NLREFVEKNIDGYFKIYSKHTPINLVRDLPQGESALEPLVDLPIGINIT | |
| | RFQTLLALHRSYLTPGDSSSGWTAGAAAYYVGYLQPRTELLKYNENGTI | |
| | TDAVDCALDPLSETKCTLKSFTVEKGIYQTSNERVQPTESIVREPNITN | |
| | LCPFGEVENATRFASVYAWNRKRISNCVADYSVLYNSASFSTEKCYGVS | |
| | PTKLNDLCFTNVYADSFVIRGDEVRQIAPGQTGKIADYNYKLPDDETGC | |
| | VIAWNSNNLDSKVGGNYNYLYRLERKSNLKPFERDISTEIYQAGSTPCN | |
| | GVEGFNCYFPLQSYGFQPTNGVGYQPYRVVVLSFELLHAPATVCGPKKS | |
| | TNLVKNKCVNFNENGLTGTGVLTESNKKELPFQQFGRDIADTTDAVRDP | |
| | QTLEILDITPCSFGGVSVITPGTNTSNQVAVLYQDVNCTEVPVAIHADQ | |
| | LTPTWRVYSTGSNVFQTRAGCLIGAEHVNNSYECDIPIGAGICASYQTQ | |
| | TNSPRRARSVASQSIIAYTMSLGAENSVAYSNNSIAIPTNFTISVTTEI | |
| | LPVSMTKTSVDCTMYICGDSTECSNLLLQYGSFCTQLNRALTGIAVEQD | |
| | KNTQEVFAQVKQIYKTPPIKDFGGFNFSQILPDPSKPSKRSFIEDLLEN | |
| | KVTLADAGFIKQYGDCLGDIAARDLICAQKENGLTVLPPLLTDEMIAQY | |
| | TSALLAGTITSGWTFGAGAALQIPFAMQMAYRENGIGVTQNVLYENQKL | |
| | IANQFNSAIGKIQDSLSSTASALGKLQDVVNQNAQALNTLVKQLSSNEG | |
| | AISSVLNDILSRLDKVEAEVQIDRLITGRLQSLQTYVTQQLIRAAEIRA | |
| | SANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITTD | |
| | NTFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPWYI | |
| | WLGFIAGLIAIVMVTIMLCCMTSCCSCLKGCCSCGSCCKEDEDDSEPVL | |
| | KGVKLHYT | |
| SARS-CoV-2 | SAESKYGPPCPPCPAPEAAGGPSVFLFPPKPKDTLMISRTPEVTCVVVD | 469 |
| Spike Fc | VSQEDPEVQFNWYVDGVEVHNAKTKPREEQFNSTYRVVSVLTVLHQDWL | |
| Fusion Protein | NGKEYKCKVSNKGLPSSIEKTISKAKGQPREPQVYTLPPSQEEMTKNQV | |
| without signal | SLTCLVKGFYPSDIAVEWESNGQPENNYKTTPPVLDSDGSFFLYSRLTV | |
| peptide | DKSRWQEGNVFSCSVMHEALHNHYTQKSLSLSLGGGGGGGGSGGGGSGG | |
| | GGSQCVNLTTRTQLPPAYTNSFTRGVYYPDKVERSSVLHSTQDLFLPFF | |
| | SNVTWFHAIHVSGTNGTKRFDNPVLPENDGVYFASTEKSNIIRGWIFGT | |
| | TLDSKTQSLLIVNNATNVVIKVCEFQFCNDPFLGVYYHKNNKSWMESEF | |
| | RVYSSANNCTFEYVSQPFLMDLEGKQGNEKNLREFVEKNIDGYFKIYSK | |
| | HTPINLVRDLPQGFSALEPLVDLPIGINITRFQTLLALHRSYLTPGDSS | |
| | SGWTAGAAAYYVGYLQPRTELLKYNENGTITDAVDCALDPLSETKCTLK | |
| | SFTVEKGIYQTSNERVQPTESIVREPNITNLCPFGEVENATRFASVYAW | |
| | NRKRISNCVADYSVLYNSASFSTFKCYGVSPTKLNDLCFTNVYADSFVI | |
| | RGDEVRQIAPGQTGKIADYNYKLPDDFTGCVIAWNSNNLDSKVGGNYNY | |
| | LYRLERKSNLKPFERDISTEIYQAGSTPCNGVEGENCYFPLQSYGFQPT | |
| | NGVGYQPYRVVVLSFELLHAPATVCGPKKSTNLVKNKCVNFNENGLTGT | |
| | GVLTESNKKELPFQQFGRDIADTTDAVRDPQTLEILDITPCSFGGVSVI | |
| | TPGTNTSNQVAVLYQDVNCTEVPVAIHADQLTPTWRVYSTGSNVFQTRA | |
| | GCLIGAEHVNNSYECDIPIGAGICASYQTQTNSPRRARSVASQSIIAYT | |
| | MSLGAENSVAYSNNSIAIPTNFTISVTTEILPVSMTKTSVDCTMYICGD | |
| | STECSNLLLQYGSFCTQLNRALTGIAVEQDKNTQEVFAQVKQTYKTPPI | |
| | KDFGGFNFSQILPDPSKPSKRSFIEDLLENKVTLADAGFIKQYGDCLGD | |
| | IAARDLICAQKENGLTVLPPLLTDEMIAQYTSALLAGTITSGWTFGAGA | |
| | ALQIPFAMQMAYRENGIGVTQNVLYENQKLIANQFNSAIGKIQDSLSST | |
| | ASALGKLQDVVNQNAQALNTLVKQLSSNFGAISSVLNDILSRLDKVEAE | |
| | VQIDRLITGRLQSLQTYVTQQLIRAAEIRASANLAATKMSECVLGQSKR | |
| | VDFCGKGYHLMSFPQSAPHGVVFLHVTYVPAQEKNFTTAPAICHDGKAH | |
| | FPREGVFVSNGTHWFVTQRNFYEPQIITTDNTFVSGNCDVVIGIVNNTV | |
| | YDPLQPELDSFKEELDKYFKNHTSPDVDLGDISGINASVVNIQKEIDRL | |
| | NEVAKNLNESLIDLQELGKYEQYIKWPWYIWLGFIAGLIAIVMVTIMLC | |
| | CMTSCCSCLKGCCSCGSCCKFDEDDSEPVLKGVKLHYT | |

Briefly, human PBMCs from each donor were thawed using standard methods and plated at a concentration of 200,000 cells/well in IMDM media with 10% heat-inactivated fetal bovine serum (FBS) in 96 well plates. The cells were stimulated with a dose of 1 nM (˜50 ng/mL) of one of the following hIL-10R agonists: hIL-10R BFP-1 (Fc-hIL-10) (SEQ ID NO: 413); hIL-10R BFP-2 (SEQ ID NO: 415); or hIL-10R BFP-10 (SEQ ID NO: 417) (see Table 9) with or without 1 ng/mL SARS-CoV-2 Spike Fc fusion protein (see Table 10) (Fc-Spike protein ˜5.9 pM) or 0.1 ug/mL CD40L (Fisher Scientific #NC9975949); the cells were incubated at 37° C., 5% CO₂ for 12 days. After 12 days, the cells were centrifuged for 1 minute at 500 g and the supernatant was removed for Ig flow cytometry analysis. Antibody concentrations in the supernatants were determined using the LegendPlex Mix and Match SARS-CoV-2 Serological IgG Panel: SARS-CoV-2 S1 Spike Protein (Cat #: 741135), SARS-CoV-2 S Protein Receptor Binding Domain (RBD) (Cat #: 741136), SARS-CoV-2 Nucleocapsid (Cat #: 741137), and SARS-CoV-2 Serological IgA Panel: SARS-CoV-2 S1 Spike Protein (Cat #: 741135) & SARS-CoV-2 S Protein RBD (Cat #: 741136), per manufacturer's protocol. Briefly, supernatants, capture beads, and assay buffer were added to 96-well plates and incubated at room temperature while shaking for 2 hours. Plates were washed, detection antibodies added, and incubated at room temperature while shaking for 1 hour. Detection reagent SA-PE (Streptavidin, R-Phycoerythrin Conjugate) was added and the plates incubated at room temperature while shaking for 30 minutes. The capture beads were spun down and resuspended in wash buffer for flow cytometry analysis. Flow cytometry analysis was performed using a Cytoplex flow cytometer and Qognit software.

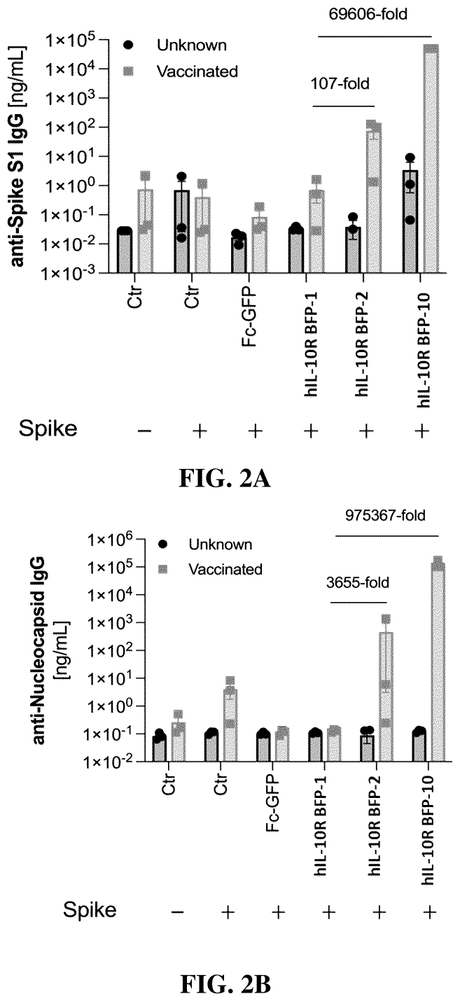

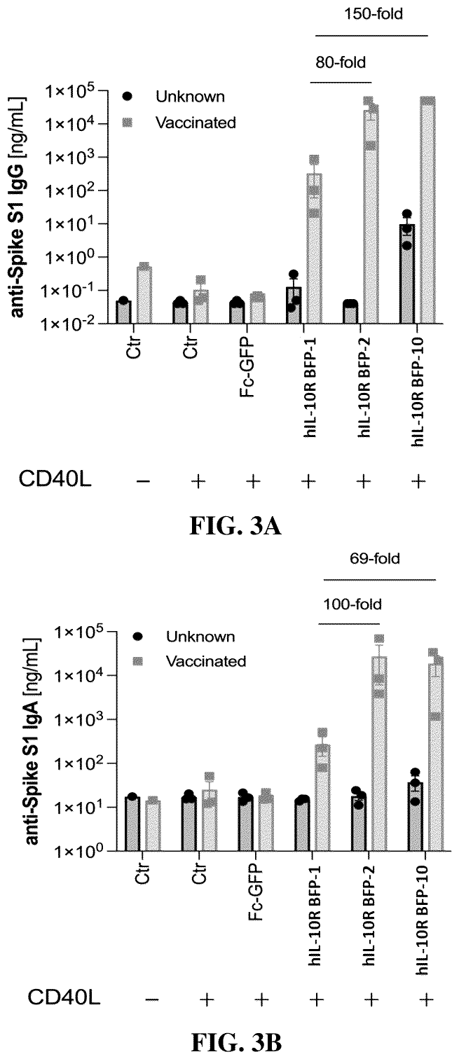

As shown in FIGS. 2A-2B, hIL-10R BFP-2 and hIL-10R BFP-10 stimulated antigen-specific IgG antibody production (anti-Spike S1 IgG (FIG. 2A) and anti-Nucleocapsid IgG (FIG. 2B)) from donor PBMCs obtained from the vaccinated and treated with SARS-Cov-2 Spike protein. As shown in FIGS. 3A-3B, hIL-10R BFP-1, hIL-10R BFP-2, and hIL-10R BFP-10 stimulated antigen-specific IgG and IgA antibody production (anti-Spike S1 IgG (FIG. 3A) and anti-Spike S1 IgA (FIG. 3B)) from donor PBMCs treated with CD40L and obtained from the vaccinated donor.

### 6.4 Example 4. In vitro B Cell Stimulation and Analysis of B Cell Populations

The generation of B cell subpopulations (e.g., plasmablasts) from PBMCs treated with one of the following hIL-10R binding fusion proteins: hIL-10R BFP-1 (Fc-hIL-10) (SEQ ID NO: 413); hIL-10R BFP-2 (SEQ ID NO: 415); and hIL-10R BFP-10 (SEQ ID NO: 417) (see Table 9), with or without antigen (i.e., SARS-Cov-2 Sike protein antigens) stimulation was assessed.

Briefly, human donor PBMCs were thawed by standard methods and plated at a concentration of 200,000 cells/well in IMDM media with 10% heat-inactivated FBS in 96 well plates. The cells were stimulated with a dose of 1 nM (˜50 ng/mL) of hIL-10R BFP-1; hIL-10R BFP-2; hIL-10R BFP-10, or the indicated control and plus or minus 0.6 Nm PepTivator SARS-CoV-2 Prot_S Complete from Miltenyi (Cat #130-127-951), and incubated at 37° C., 5% CO₂ for 12 days. After 12 days, the cells were washed in PBS and stained with a viability dye. The cells were washed in Flow Buffer (DPBS+5% heat-inactivated FBS) and blocked with mouse serum and Fc block. The cells were subsequently stained with the B cell identification antibodies listed below for 30 minutes in the dark at room temperature. The B cell identification antibodies from TFS were anti-CD20 BV421 (Cat #404-0209-42), anti-CD19 SB780 (Cat #78-0199-42), and anti-CD38 APC (Cat #17-0389-42). Cells were washed in Flow Buffer and analyzed with an Attune NXT flow cytometer. Data was analyzed in Flowjo and Prism.

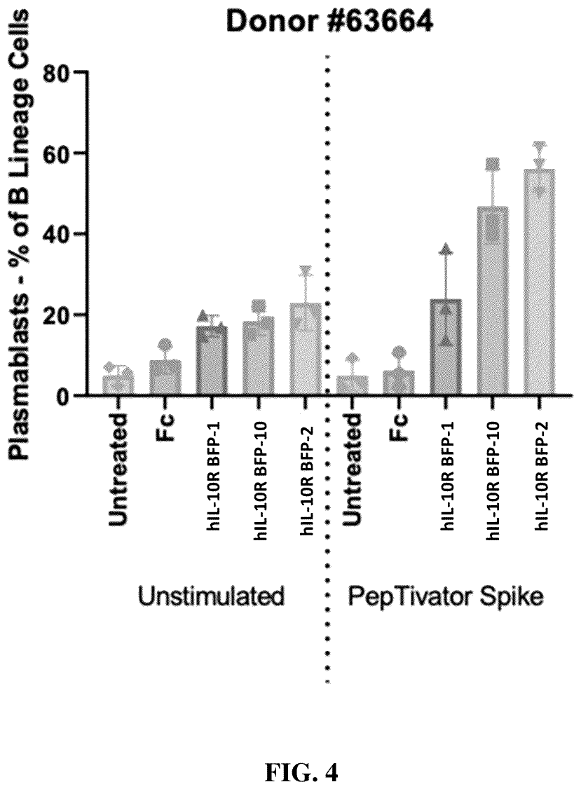

As shown in FIG. 4, each of the hIL-10R binding fusion proteins assessed stimulated an increase in the percentage of plasmablasts in the PBMC cultures, particularly in the presence of antigen.

### 6.5 Example 5. Differential hIL-10R Subunit Expression and Binding by hIL-10R Binding Agents Described Herein

Expression of the hIL-10Rα and hIL-10Rβ subunits by various immune cell populations was assessed in conjunction with the binding affinity of the hIL-10R BFP-1 (SEQ ID NO: 522) or hIL-10R BFP-10 (SEQ ID NO: 534) to each receptor subunit.

The level of expression of the hIL-10Rα subunit and hIL-10Rβ subunit by different populations of immune cells (namely, CD14+ monocytes, B cells, CD4+ T cells, and CD8+ T cells) was assessed by flow cytometry performed on human PBMCs from 5 different donors. A mouse monoclonal anti-human hIL-10Rβ antibody (allophycocyanin conjugate) (R&D Systems Catalog Number: FAB874A; Clone #90220) and a rat anti-human hIL-10Rα antibody (phycoerythrin conjugate) (BD Pharmingen; Material Number 556013; Clone 3F9) were utilized for the flow cytometry analysis.

The binding affinity of hIL-10R BFP-1 and hIL-10R BFP-10 assessed by ELISA. Briefly, 384-Maxisorp plates (Sigma, P6366-1CS) were coated with 25 μL per well of either the a or R subunit of the hIL-10R at a concentration of 2 μg/mL in 1× coating buffer (VWR, 421701-BL). The plates were then stored overnight at 4° C. The following day, they were washed three times using 80 μL per well of 0.05% PBS-T (Fisher, PI28352). Next, 80 μL of SUPER BLOCK buffer (Fisher, NC9782835) was added to each well, and the plates were incubated on a shaker at 400 RPM for 1 hour at room temperature (RT). After this incubation, the plates were washed as previously described. Then, 25 μL of supernatant protein was added to each well, followed by another incubation on the shaker at 400 RPM for 1 hour at RT. Another wash was performed as before. Subsequently, 25 μL of Goat anti-Human IgG (H+L) Secondary Antibody, HRP (Thermofisher, H10307), diluted at 1:2000 in SUPER BLOCK, was added to each well and incubated for 1 hour at RT on the shaker. Following this, the plates were washed again as previously. 20 μL of TMB substrate (Thermofisher, 34028) was then added to each well, and allowed to sit for 2 minutes in the dark at RT. Finally, 20 μL of Stop Solution (BioFX, LSTP-1000-01) was added to all wells, and the absorbance at 450 nm was measured using a Varioskan.

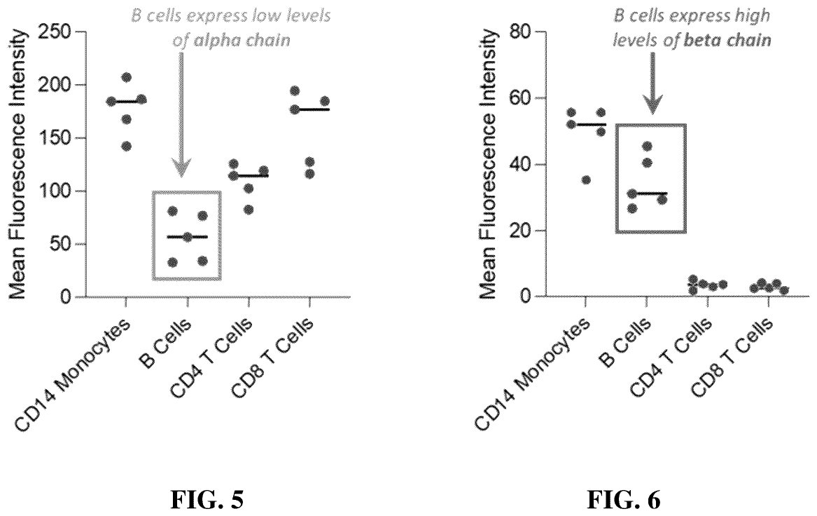

Monocytes expressed the highest level of expression of both the hIL-10Rα subunit and the hIL-10Rβ subunit (FIG. 5 and FIG. 6). The adaptive immune cell populations (B cells, CD4+ T cells, and CD8+ T cells) showed variable subunit expression (FIG. 5 and FIG. 6). B cells expressed moderate level of the hIL-10R3 subunit (FIG. 6) and low levels of the hIL-10Rα subunit (FIG. 5). CD4+ T cells and CD8+ T cells showed moderate to high levels of expression of the hIL-10Rα subunit (FIG. 5) and no expression of the hIL-10Rβ subunit (FIG. 6).

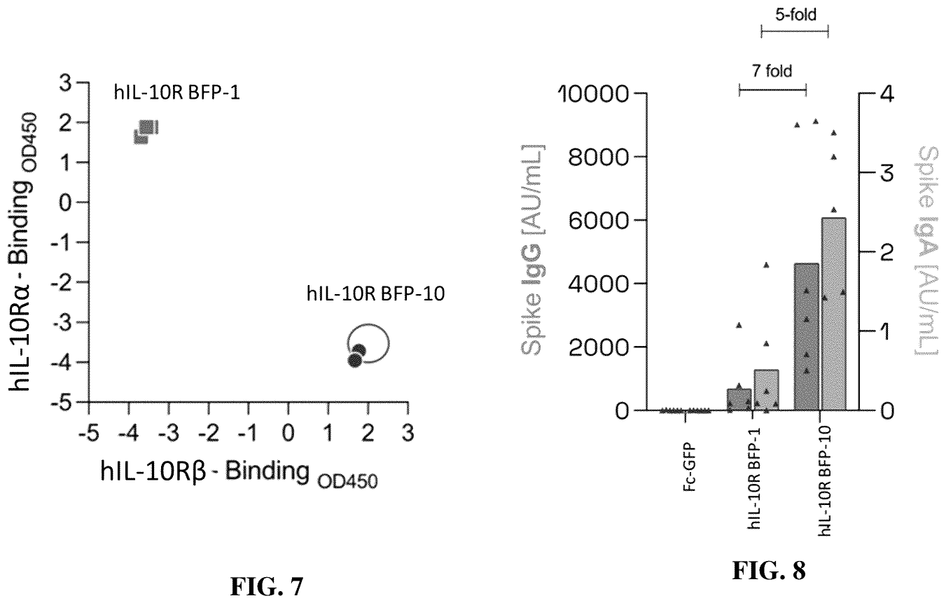

Furthermore, hIL-10R BFP-10 exhibited high binding affinity for the hIL-10Rβ subunit and low binding affinity for the hIL-10Rα subunit (FIG. 7) (e.g., relative to the hIL-10R BFP-1 fusion protein which showed a significantly higher binding affinity for the hIL-10Rα subunit and significantly lower binding affinity for the hIL-10Rβ subunit (FIG. 7)).

Together, the data shows that differential expression of the hIL-10 receptor subunits (i.e., the hIL-10Rα chain and the hIL-10Rβ chain) by various immune cell subtypes provides a novel approach to selectively augment B cell responses through the use of hIL-10R binding agents described herein (including, e.g., hIL-10R BP-10 (including, e.g., fusion proteins thereof)) with the ability to bind the hIL-10Rβ chain with high affinity (e.g., higher affinity relative to affinity for the hIL-10Rα chain).

### 6.6 Example 6. In Vitro Enhancement of B Cell Responses Through the Promotion of T Cell Help

The in vitro production of antigen specific IgG and IgA antibodies by antigen stimulated B cells treated with hIL-10R BFP-1 (SEQ ID NO: 522) or hIL-10R BFP-10 (SEQ ID NO: 534) was assessed; in conjunction with the induction of long-lived plasma cell differentiation by hIL-10R BFP-10.

Briefly, human donor PBMCs were thawed using standard methods and then plated at a concentration of 200,000 cells/well in IMDM media with 10% heat-inactivated serum in a 96 well plate. The cells were treated with protein supernatant and stimulated with 100 ng/mL of Spike peptides (PepTivator Prot Spike Complete, Miltenyi Biotec, 130-127-951), followed by incubation at 37° C., 5% CO₂ for 12 days. On the 12th day, the cells were centrifuged for 5 minutes at 500 g, after which the supernatant was removed for anti-Spike IgG antibody analysis. Subsequently, the cells were stained with antibodies for a multi-parametric analysis of single cells in solution, focusing on plasmablasts differentiation, using an Attune instrument.

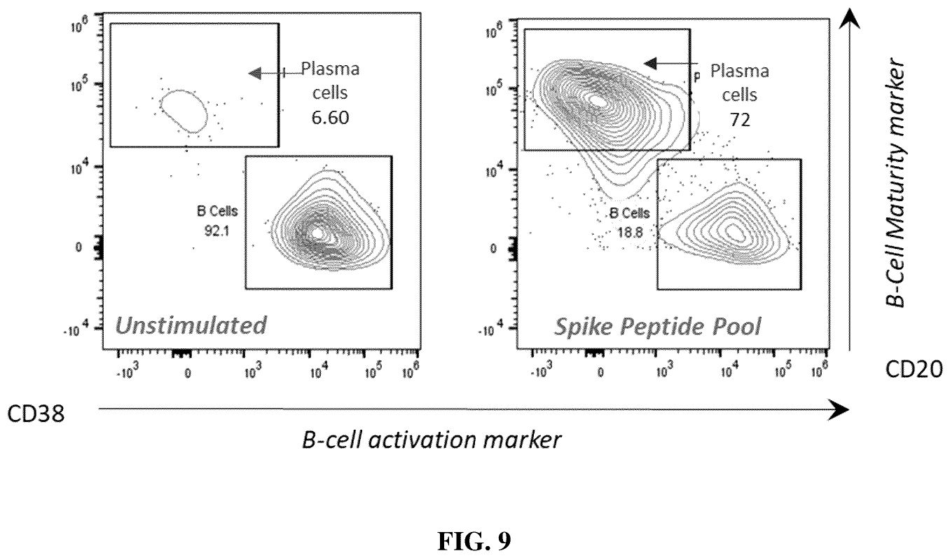

hIL-10R BFP-10 induced antigen (i.e., SARS-CoV-2 spike protein) specific IgG (left bar for each control or treatment group) and IgA (right bar for each control or treatment group) antibodies (FIG. 8) (seven-fold higher antigen specific IgG production and five-fold higher IgA production compared to hIL-10R BFP-1. Furthermore, hIL-10R BFP-10 induced long term plasma cell maturation in antigen (SARS-CoV-2 spike protein) stimulated B cell populations (hIL-10R BFP-10 treated and stimulated B cell population comprising ˜72% plasma cells and ˜18% other B cells (FIG. 9 (right plot)); and hIL-10R BFP-10 treated and unstimulated B cell population comprising ˜6% plasma cells and ˜92% other B cells (FIG. 9 (left plot))).

Together, the data shows that hIL-10R binding agents described herein (including, e.g., hIL-10R BP-10 (including, e.g., fusion proteins thereof)) augment antigen-specific B-cell expansion driving IgG and IgA production as well as long-term plasma cell maturation.

### 6.7 Example 7. Suppression of Immune Cell Functions Associated with Vaccine Reactogenicity

The expression of cytokines associated with vaccine reactogenicity by stimulated T cells and monocytes treated with hIL-10R BFP-1 (SEQ ID NO: 522) or hIL-10R BFP-10 (SEQ ID NO: 534) was assessed.

The induced suppression of cytokine production from SARS-Cov-2 spike protein stimulated hPBMCs was assessed as follows. Briefly, human donor PBMCs were thawed following standard methods and plated at a concentration of 200,000 cells/well in IMDM media with 10% heat inactivated serum in a 96-well plate. The cells were treated with protein supernatant (hIL-10R BFP-1, hIL-10R BFP-10, an Fc-GFP control (SEQ ID NO: 419), or untreated) and stimulated with 100 ng/mL of Spike peptides (PepTivator Prot Spike Complete, Miltenyi Biotec, 130-127-951), and then incubated at 37° C. with 5% CO₂ for 12 days. After the initial 24 hours, a small sample (approximately 2 μL) of the supernatant was removed for cytokine analysis. The concentration of cytokine (IFNγ) in the supernatant from the stimulated human PBMCs was determined using the V-Plex Proinflammatory Panel 2 Human Kit (Mesoscale Diagnostics #K15346D-2), following the manufacturer's protocol.

The induced suppression of cytokine production from LPS-stimulated human PBMCs was assessed as follows. Briefly, human donor PBMCs were thawed using standard methods and then plated at a concentration of 200,000 cells/well in HPLM media supplemented with 2% type AB human serum in a 96-well plate. The cells were pretreated with protein supernatant (hIL-10R BFP-1, hIL-10R BFP-10, or untreated) and incubated at 37° C. with 5% CO₂ for 20 minutes. Following the pretreatment, the cells were stimulated with 1 ng/mL of LPS (Thermofisher #00-4976-03) and then incubated at the same temperature and CO₂ conditions for 24 hours. After this 24-hour period, the cells were centrifuged for 3 minutes at 500 g, and the supernatant was collected for multiplex ELISA analysis. The cytokine concentrations (IL-10, IL-6) in the supernatant from the stimulated human PBMCs were determined using the V-Plex Proinflammatory Panel 2 Human Kit (Mesoscale Diagnostics #K15346D-2), in accordance with the manufacturer's protocol.

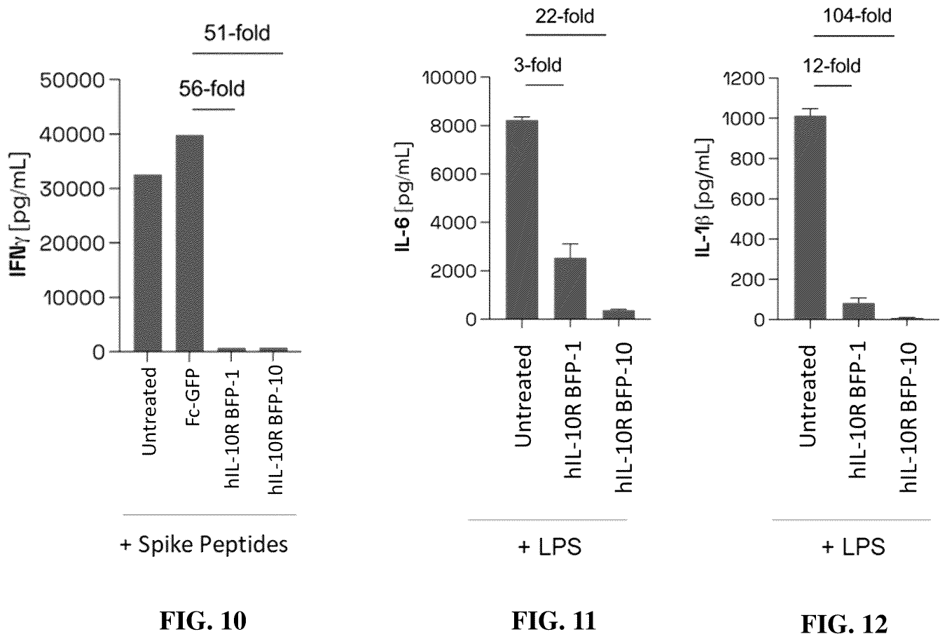

hIL-10R BFP-10 suppressed production of IFNγ from T cells (a 51-fold decrease relative to the Fc-GFP control) (FIG. 10), indicative of suppressed antigen presentation by monocytes. Furthermore, hIL-10R BFP-10 suppressed production of IL-6 (a 22-fold decrease relative to the untreated control) (FIG. 11) and IL-1 from monocytes (a 104-fold decrease relative to the untreated control) (FIG. 12) from monocytes, indicative of suppression of LPS induced stimulation of monocytes.

Collectively, the data shows that hIL-10R BFP-10 induces limited production of pro-inflammatory cytokines, such as IFNγ, IL-6, and IL-10, that are associated with vaccine reactogenicity.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1.-113. (canceled)

114. A combination therapy comprising
(a) a nucleic acid molecule comprising a coding region encoding a SARS-CoV-2 immunogenic protein; and
(b) a nucleic acid molecule comprising a coding region encoding a hIL-10R binding protein, wherein the amino acid sequence of the encoded hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-353.

115. A combination therapy comprising
(a) an immunogenic protein (or an immunogenic fragment and/or immunogenic variant thereof) or a nucleic acid molecule comprising a coding region encoding an immunogenic protein (or an immunogenic fragment and/or immunogenic variant thereof); and
(b) a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof).

116. A kit comprising the combination therapy of claim 115.

117. A vaccine composition comprising
(a) a nucleic acid molecule comprising a coding region encoding a SARS-CoV-2 immunogenic protein; and
(b) a nucleic acid molecule comprising a coding region encoding a hIL-10R binding protein, wherein the amino acid sequence of the encoded hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-353.

118. A vaccine composition comprising
(a) an immunogenic protein (or an immunogenic fragment and/or immunogenic variant thereof) or a nucleic acid molecule comprising a coding region encoding an immunogenic protein (or an immunogenic fragment and/or immunogenic variant thereof); and
(b) a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof).

119. A vaccine composition, wherein the improvement comprises a human IL-10 Receptor (hIL-10R) binding protein (or a functional fragment and/or functional variant thereof) or a nucleic acid molecule comprising a coding region encoding a hIL-10R binding protein (or a functional fragment and/or functional variant thereof) that enhances an immune response when administered to a subject.

120. A pharmaceutical composition comprising the vaccine composition of claim 118.

121. A cell comprising the vaccine composition of claim 118.

122. A kit comprising the vaccine composition of claim 118.

123. A nucleic acid molecule comprising
(a) a coding region encoding a first immunogenic protein (or an immunogenic fragment and/or immunogenic variant thereof) and
(b) a coding region encoding a hIL-10R binding protein (or a functional fragment and/or functional variant thereof).

124. A vector comprising the nucleic acid molecule of claim 123.

125. A carrier comprising the nucleic acid molecule of claim 123.

126. A cell comprising the nucleic acid molecule of claim 123.

127. A pharmaceutical composition comprising the nucleic acid molecule of claim 123.

128. A vaccine composition comprising the nucleic acid molecule of claim 123.

129. A kit comprising the nucleic acid molecule of claim 123.

130. A method of vaccinating a subject, the method comprising administering to the subject
(a) a nucleic acid molecule comprising a coding region encoding a SARS-CoV-2 immunogenic protein; in combination with
(b) a nucleic acid molecule comprising a coding region encoding a hIL-10R binding protein,
wherein the amino acid sequence of the encoded hIL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-353,
to thereby vaccinate the subject.

131. A method of vaccinating a subject, the method comprising administering to the subject
(a) an immunogenic protein (or an immunogenic fragment and/or immunogenic variant thereof) or a nucleic acid molecule comprising a coding region encoding an immunogenic protein (or an immunogenic fragment and/or immunogenic variant thereof); in combination with
(b) a hIL-10R binding protein (or a functional fragment and/or functional variant thereof) or a nucleic acid molecule comprising a coding region encoding a hIL-10R binding protein (or a functional fragment and/or functional variant thereof),
to thereby vaccinate the subject.

132. A method of treating a subject exposed to an infective agent, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby treat the subject.

133. A method of ameliorating, treating, or preventing an infection in a subject, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the infection in the subject.

134. The method of claim 133, wherein the infection is an acute infection.

135. A method of ameliorating, treating, or preventing a disease associated with an infection, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, treat, or prevent the disease associated with the infection in the subject.

136. The method of claim 135, comprising ameliorating, treating, or preventing a severe form of the disease associated with the infection.

137. The method of claim 135, wherein the disease is post viral syndrome.

138. A method of enhancing an immunogen-specific immune response in a subject, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby enhance the immunogen specific immune response in the subject.

139. A method of increasing the level of immunogen-specific IgG or IgA in a subject, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby increasing the level of immunogen-specific IgG or IgA in the subject.

140. The method of claim 139, wherein the method comprises increasing the level of immunogen-specific mucosal IgA.

141. A method of promoting the generation of, enhancing the generation of, and/or sustaining the level of plasma cells in a subject, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby promote the generation of, enhance the generation of, and/or sustain the level of plasma cells in the subject.

142. A method of ameliorating, reducing, or preventing reactogenicity induced by administration of a vaccine, the method comprising administering to the subject a hIL-10R binding protein (or a functional fragment and/or functional variant thereof) or a nucleic acid molecule comprising a coding region encoding a hIL-10R binding protein (or a functional fragment and/or functional variant thereof), to thereby ameliorate, reduce, or prevent reactogenicity induced by administration of the vaccine in the subject.

143. A method of making a lipid nanoparticle, the method comprising combining one or more lipid and a hIL-10R binding protein (or a functional fragment and/or functional variant thereof) or a nucleic acid molecule comprising a coding region encoding a hIL-10R binding protein (or a functional fragment and/or functional variant thereof), wherein the hIL-10R binding protein or the nucleic acid molecule is encapsulated within the lipid nanoparticle.


SEQUENCE LISTING (572 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, Homo sapiens
MHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 3 - protein, 178 aa, Homo sapiens
MHSSALLCCLVLLTGVRASPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 180 aa, Homo sapiens
MGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 5 - protein, 197 aa, Homo sapiens
MARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 6 - protein, 186 aa, Homo sapiens
MSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 7 - protein, 199 aa, Homo sapiens
MANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 8 - protein, 182 aa, Homo sapiens
MQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 9 - protein, 187 aa, Homo sapiens
MRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 10 - protein, 185 aa, Homo sapiens
MGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 11 - protein, 175 aa, Homo sapiens
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 12 - protein, 182 aa, Homo sapiens
MALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 13 - protein, 189 aa, Homo sapiens
MRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 14 - protein, 186 aa, Homo sapiens
MRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 15 - protein, 185 aa, Homo sapiens
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 16 - protein, 185 aa, Homo sapiens
MSNNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 17 - protein, 185 aa, Homo sapiens
MPNNKILVCAVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 18 - protein, 184 aa, Homo sapiens
MSNKKILVCVVIILTYTLYTDAYCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 19 - protein, 185 aa, Homo sapiens
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 20 - protein, 185 aa, Homo sapiens
MSKNKVLVCFVIILTYTLYTDAYCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 21 - protein, 185 aa, Homo sapiens
MSNNKILVCAVIILTYTLYTDAYCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 22 - protein, 186 aa, Homo sapiens
MSNNKILLCVAIILTYTLYTDAYCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 23 - protein, 185 aa, Homo sapiens
MSNNKILVCAVIILTYTLYTDAYCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 24 - protein, 185 aa, Homo sapiens
MSKNKFLVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 25 - protein, 186 aa, Homo sapiens
MSKNKILVCFVIILTYTLYTDAYCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 26 - protein, 185 aa, Homo sapiens
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 27 - protein, 186 aa, Homo sapiens
MSNNKILVCVVIILTYTLYTDAYCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 28 - protein, 186 aa, Homo sapiens
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 29 - protein, 186 aa, Homo sapiens
MSKNKILVCVAIILTYTLYTDAYCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 30 - protein, 186 aa, Homo sapiens
MSKNKILVCVAIILTYTLYTDAYCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 31 - protein, 185 aa, Homo sapiens
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 32 - protein, 186 aa, Homo sapiens
MSNNKILVCVVIILTYTLYTDAYCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 33 - protein, 186 aa, Homo sapiens
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 34 - protein, 179 aa, Homo sapiens
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 35 - protein, 186 aa, Homo sapiens
MSKNKILVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 36 - protein, 186 aa, Homo sapiens
MSKNKILVCVAIILTYTLYTDAYCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 37 - protein, 186 aa, Homo sapiens
MSKNKILVCVAIILTYTLYTDAYCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 38 - protein, 186 aa, Homo sapiens
MSKNKILVCVAIILTYTLYTDAYCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 39 - protein, 178 aa, Homo sapiens
MPGAALLYCLFFVTGVWAESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 40 - protein, 186 aa, Homo sapiens
MSNNKILVCVAIILTYTLYTDAYCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 41 - protein, 185 aa, Homo sapiens
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 42 - protein, 185 aa, Homo sapiens
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 43 - protein, 174 aa, Homo sapiens
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 44 - protein, 174 aa, Homo sapiens
ILVCFVIILTYTLYTDAYCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 45 - protein, 174 aa, Homo sapiens
LVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 46 - protein, 177 aa, Homo sapiens
MGLRSGLTLQCLVILQCLVMLYLAPACKGVSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 47 - protein, 174 aa, Homo sapiens
LVCVAIILTYTLYTDAYCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 48 - protein, 174 aa, Homo sapiens
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 49 - protein, 186 aa, Homo sapiens
MANVIYVVLALNILLSQIHVSNPYCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 50 - protein, 174 aa, Homo sapiens
ILVCFVIILTYTLYTDAYCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 51 - protein, 189 aa, Homo sapiens
MANVIYVVLALNILLSQFHVSNPYCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 52 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 53 - protein, 170 aa, Homo sapiens
MERRLVVTLQCLVLLYLAPECGSTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 54 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 55 - protein, 171 aa, Homo sapiens
MGPRAGLALQCLLLLYLAPACKGVSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 56 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 57 - protein, 170 aa, Homo sapiens
MERRLVVTLQCLVLLYLAPECGGTDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 58 - protein, 170 aa, Homo sapiens
MERRLMVTLQCLVLLYLAPECGSTDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 59 - protein, 170 aa, Homo sapiens
MERRLVVTLQCLVLLYLAPECGGTDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 60 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 61 - protein, 170 aa, Homo sapiens
MERRLMVTLQCLVLLYLAPECGSTDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 62 - protein, 177 aa, Homo sapiens
MELRSGLTLQCLVMLQCLVMLYLAPACKGASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 63 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 64 - protein, 161 aa, Homo sapiens
MERRLVVTLQCLVLLYLAPECGEMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 65 - protein, 171 aa, Homo sapiens
MELSLGLTLHFLVFLCLAPACGRAETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 66 - protein, 204 aa, Homo sapiens
MAYGKKIVAASLLVIPAYVVFTNATANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 67 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 68 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 69 - protein, 175 aa, Homo sapiens
MLSVMVSSSLVLIVFFLGASEEAKPATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 70 - protein, 176 aa, Homo sapiens
MLSVMVSSSLVLIVFFLGASEEAKPATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 71 - protein, 176 aa, Homo sapiens
MLSVMVSSSLVLIVFFLGASEEAKPATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 72 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 73 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 74 - protein, 176 aa, Homo sapiens
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 75 - protein, 176 aa, Homo sapiens
MLSVMVSSSLVLIVFFLGASEEAKPATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 76 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 77 - protein, 176 aa, Homo sapiens
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 78 - protein, 175 aa, Homo sapiens; identical to SEQ ID NO: 11

SEQ ID NO: 79 - protein, 176 aa, Homo sapiens
MLSVMVSSSLVLIVFFLGASEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 80 - protein, 176 aa, Homo sapiens
MLSVMVSSSLVLIVFFLGASEEAKPAATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 81 - protein, 176 aa, Homo sapiens
MLSVMVSSSLVLIVFFLGASEEAKPATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 82 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 83 - protein, 176 aa, Homo sapiens
MLSVMVSSSLVLIVFFLGASEEAKPATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 84 - protein, 175 aa, Homo sapiens
MLSVMVSSSLVLIVFFLGASEEAKPATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 85 - protein, 176 aa, Homo sapiens
MLSVMVSSSLVLIVFFLGASEEAKPATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 86 - protein, 177 aa, Homo sapiens
MLSVMVFSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 87 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 88 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 89 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 90 - protein, 175 aa, Homo sapiens
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 91 - protein, 177 aa, Homo sapiens
MLSVMVSSSLVLIVFFLGASEEAKPAATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 92 - protein, 178 aa, Homo sapiens
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 93 - protein, 177 aa, Homo sapiens
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 94 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 95 - protein, 175 aa, Homo sapiens
MLSVMVSSSLVLIVFFLGASEEAKSATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 96 - protein, 176 aa, Homo sapiens
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 97 - protein, 175 aa, Homo sapiens
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 98 - protein, 177 aa, Homo sapiens
MLSVMVSSSLVLIVFFLGASEEAKPATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 99 - protein, 178 aa, Homo sapiens
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 100 - protein, 178 aa, Homo sapiens
MLSVMVSSSLVLIIFFLGASEEAKPATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 101 - protein, 176 aa, Homo sapiens
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 102 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 103 - protein, 176 aa, Homo sapiens
MLSVMVSSSLVLIVFFLGASEEAKPATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 104 - protein, 179 aa, Homo sapiens
MLSVMVSSSLVLIVFFLGASEEAKPATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 105 - protein, 177 aa, Homo sapiens
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 106 - protein, 175 aa, Homo sapiens
MLSVMVSSSLVLIVFFLGASEKAKSATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 107 - protein, 179 aa, Homo sapiens
MLSVMVSSSLVLIVFFLGASEEAKPAATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 108 - protein, 179 aa, Homo sapiens
MLSVMVSSSLVLIVFFLGASEEAKPAATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 109 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 110 - protein, 177 aa, Homo sapiens
MLSVMVSSSLVLIVFFLGASEEAKPAATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 111 - protein, 173 aa, Homo sapiens
MLSVMVSSSLVLIVFFLGASEEAKPATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 112 - protein, 180 aa, Homo sapiens
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 113 - protein, 180 aa, Homo sapiens
MLSVMVSSSLVLIVFFLGASEEAKPAATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 114 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 115 - protein, 181 aa, Homo sapiens
MLSVMVSSSLVLIVFFLGASEEAKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 116 - protein, 179 aa, Homo sapiens
MLSVMVSSSLVLIVFFLGASEEAKPAATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 117 - protein, 177 aa, Homo sapiens
MLSVMVSSSLVMIVFFLGASEEAKPATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 118 - protein, 182 aa, Homo sapiens
MLSVMVSSSLVLIVFFLGASEEAKPAAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 119 - protein, 180 aa, Homo sapiens
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 120 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 121 - protein, 177 aa, Homo sapiens
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 122 - protein, 182 aa, Homo sapiens
MLSVMVSSSLVLIVFFLGASEEAKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 123 - protein, 176 aa, Homo sapiens
MLSVMVSSSLVLIVFFLGASEEAKPATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 124 - protein, 170 aa, Homo sapiens
MKTNTKIILFCYVILSLYVFSCAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 125 - protein, 170 aa, Homo sapiens
MKTNTKIILFCYVILSLYVFSCAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 126 - protein, 171 aa, Homo sapiens
MKTNTKIILFCYVIFLSLYVFSCVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 127 - protein, 170 aa, Homo sapiens
MKTNTKIILFCYVILSLYVFSCVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 128 - protein, 170 aa, Homo sapiens
MKTSTKIILFCYVILSLYVFSCVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 129 - protein, 168 aa, Homo sapiens
MKTNTKIILFCYVILYVFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 130 - protein, 170 aa, Homo sapiens
MKTSTKIILFCYVILSLYVFSCVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 131 - protein, 168 aa, Homo sapiens
MKTNTKIILFCYVILYLFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 132 - protein, 174 aa, Homo sapiens
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 133 - protein, 168 aa, Homo sapiens
MKTNTKIILFCYVILYLFSCTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 134 - protein, 150 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 135 - protein, 185 aa, Homo sapiens
MGSRRLSRCSLATAVCLVAIVVAVAAKGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 136 - protein, 159 aa, Homo sapiens
MLSVMVSSSLVLIVFLLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 137 - protein, 187 aa, Homo sapiens
MRRRRSFGVVVVGAIGTLLMMAVVVLSAHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 138 - protein, 188 aa, Homo sapiens
MGSRPARMCGLSNLLCLLLVVLVAVVIHRGCGASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 139 - protein, 142 aa, Homo sapiens
MLSMMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 140 - protein, 139 aa, Homo sapiens
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 141 - protein, 190 aa, Homo sapiens
MININILSLLILILSIYANAIIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 142 - protein, 192 aa, Homo sapiens
MINISINILSLLILILSIYANSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 143 - protein, 191 aa, Homo sapiens
MININILSLLILILSIYANAIIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 144 - protein, 191 aa, Homo sapiens
MININILSLLILILSIYANAIIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 145 - protein, 165 aa, Homo sapiens
MARFIYVVLLCLVFDAAQSAAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 146 - protein, 170 aa, Homo sapiens
MMLALAMMLMALGPLSTNAMYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 147 - protein, 165 aa, Homo sapiens
MARFIYVVLLCLVFDAAQSAAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 148 - protein, 168 aa, Homo sapiens
MFQLEGIVLLVYLANWVSPATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 149 - protein, 169 aa, Homo sapiens
MMTTLALVMTLMALSTNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 150 - protein, 171 aa, Homo sapiens
MITLALVMTLMALGPLSTNAMYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 151 - protein, 169 aa, Homo sapiens
MMTTLALVMTLMALATNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 152 - protein, 177 aa, Homo sapiens
MSGTSNKKFVFLVAIAVAICMMSSVSSNVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 153 - protein, 170 aa, Homo sapiens
MLALAMVLMALGPLSTNAMYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 154 - protein, 179 aa, Homo sapiens
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 155 - protein, 170 aa, Homo sapiens
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 156 - protein, 170 aa, Homo sapiens
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 157 - protein, 171 aa, Homo sapiens
MITLALVMTLMALGPLSTNGVHARRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 158 - protein, 180 aa, Homo sapiens
MDAQFLLLIVLALPASFAASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 159 - protein, 156 aa, Homo sapiens
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 160 - protein, 156 aa, Homo sapiens
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 161 - protein, 134 aa, Homo sapiens
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 162 - protein, 179 aa, Homo sapiens
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 163 - protein, 179 aa, Homo sapiens
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 164 - protein, 179 aa, Homo sapiens
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 165 - protein, 179 aa, Homo sapiens
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 166 - protein, 179 aa, Homo sapiens
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 167 - protein, 175 aa, Homo sapiens
MLSVMVSSSLVLIVFFLGASEEAKPATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 168 - protein, 176 aa, Homo sapiens
MLSVMVSSSLVLIVFFLGAFEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 169 - protein, 177 aa, Homo sapiens
MLSVMVSSSLVLIVFFLGASEEAKPATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 170 - protein, 174 aa, Homo sapiens
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 171 - protein, 171 aa, Homo sapiens
MKTNTKIILFCYVIFLSLYVFSCVVASTKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 172 - protein, 148 aa, Homo sapiens
MLSVMVSSSLVLIVFFLGASEEAKPATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 173 - protein, 190 aa, Homo sapiens
MSNFRILSLLIFSLIVHVNAMIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 174 - protein, 165 aa, Homo sapiens
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 175 - protein, 165 aa, Homo sapiens
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 176 - protein, 165 aa, Homo sapiens
MARFIYVVLLCLVFDAAQSAAQCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 177 - protein, 165 aa, Homo sapiens
MARFIYVVLLCLVFDAAQSAAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 178 - protein, 125 aa, Homo sapiens
MLSVMVSSSLVLIVFLLGASEEAKPATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 179 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 180 - protein, 160 aa, Homo sapiens
SPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 181 - protein, 160 aa, Homo sapiens
SPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 182 - protein, 154 aa, Homo sapiens
RHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 183 - protein, 171 aa, Homo sapiens
CQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 184 - protein, 164 aa, Homo sapiens
YCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 185 - protein, 176 aa, Homo sapiens
YCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 186 - protein, 161 aa, Homo sapiens
RSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 187 - protein, 157 aa, Homo sapiens
HEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 188 - protein, 159 aa, Homo sapiens
KGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 189 - protein, 150 aa, Homo sapiens
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 190 - protein, 163 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 191 - protein, 163 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 192 - protein, 163 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 193 - protein, 162 aa, Homo sapiens
YCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 194 - protein, 163 aa, Homo sapiens
YCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 195 - protein, 163 aa, Homo sapiens
YCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 196 - protein, 163 aa, Homo sapiens
YCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 197 - protein, 164 aa, Homo sapiens
YCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 198 - protein, 163 aa, Homo sapiens
YCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 199 - protein, 163 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 200 - protein, 164 aa, Homo sapiens
YCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 201 - protein, 163 aa, Homo sapiens
YCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 202 - protein, 164 aa, Homo sapiens
YCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 203 - protein, 164 aa, Homo sapiens
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 204 - protein, 164 aa, Homo sapiens
YCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 205 - protein, 164 aa, Homo sapiens
YCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 206 - protein, 163 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 207 - protein, 164 aa, Homo sapiens
YCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 208 - protein, 164 aa, Homo sapiens
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 209 - protein, 161 aa, Homo sapiens
DNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 210 - protein, 164 aa, Homo sapiens
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 211 - protein, 164 aa, Homo sapiens
YCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 212 - protein, 164 aa, Homo sapiens
YCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 213 - protein, 164 aa, Homo sapiens
YCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 214 - protein, 160 aa, Homo sapiens
ESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 215 - protein, 164 aa, Homo sapiens
YCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 216 - protein, 163 aa, Homo sapiens
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 217 - protein, 163 aa, Homo sapiens
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 218 - protein, 157 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 219 - protein, 157 aa, Homo sapiens
YCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 220 - protein, 158 aa, Homo sapiens
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 221 - protein, 148 aa, Homo sapiens
VSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 222 - protein, 158 aa, Homo sapiens
YCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 223 - protein, 157 aa, Homo sapiens
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 224 - protein, 163 aa, Homo sapiens
YCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 225 - protein, 157 aa, Homo sapiens
YCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 226 - protein, 166 aa, Homo sapiens
YCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 227 - protein, 147 aa, Homo sapiens
TDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 228 - protein, 147 aa, Homo sapiens
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 229 - protein, 147 aa, Homo sapiens
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 230 - protein, 148 aa, Homo sapiens
VSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 231 - protein, 147 aa, Homo sapiens
TDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 232 - protein, 147 aa, Homo sapiens
TDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 233 - protein, 147 aa, Homo sapiens
TDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 234 - protein, 147 aa, Homo sapiens
TDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 235 - protein, 147 aa, Homo sapiens
TDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 236 - protein, 147 aa, Homo sapiens
TDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 237 - protein, 148 aa, Homo sapiens
ASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 238 - protein, 147 aa, Homo sapiens
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 239 - protein, 139 aa, Homo sapiens
EMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 240 - protein, 147 aa, Homo sapiens
ETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 241 - protein, 180 aa, Homo sapiens
TANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 242 - protein, 151 aa, Homo sapiens
ATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 243 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 244 - protein, 150 aa, Homo sapiens
ATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 245 - protein, 151 aa, Homo sapiens
ATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 246 - protein, 151 aa, Homo sapiens
ATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 247 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 248 - protein, 151 aa, Homo sapiens
ATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 249 - protein, 151 aa, Homo sapiens
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 250 - protein, 151 aa, Homo sapiens
ATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 251 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 252 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 253 - protein, 150 aa, Homo sapiens; identical to SEQ ID NO: 189

SEQ ID NO: 254 - protein, 151 aa, Homo sapiens
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 255 - protein, 151 aa, Homo sapiens
AATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 256 - protein, 151 aa, Homo sapiens
ATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 257 - protein, 151 aa, Homo sapiens
ATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 258 - protein, 151 aa, Homo sapiens
ATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 259 - protein, 150 aa, Homo sapiens
ATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 260 - protein, 151 aa, Homo sapiens
ATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 261 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 262 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 263 - protein, 151 aa, Homo sapiens
ATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 264 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 265 - protein, 150 aa, Homo sapiens
ATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 266 - protein, 152 aa, Homo sapiens
AATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 267 - protein, 153 aa, Homo sapiens
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 268 - protein, 152 aa, Homo sapiens
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 269 - protein, 152 aa, Homo sapiens
ATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 270 - protein, 150 aa, Homo sapiens
ATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 271 - protein, 151 aa, Homo sapiens
ATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 272 - protein, 150 aa, Homo sapiens
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 273 - protein, 152 aa, Homo sapiens
ATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 274 - protein, 153 aa, Homo sapiens
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 275 - protein, 153 aa, Homo sapiens
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 276 - protein, 151 aa, Homo sapiens
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 277 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 278 - protein, 151 aa, Homo sapiens
ATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 279 - protein, 154 aa, Homo sapiens
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 280 - protein, 152 aa, Homo sapiens
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 281 - protein, 150 aa, Homo sapiens
ATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 282 - protein, 153 aa, Homo sapiens
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 283 - protein, 153 aa, Homo sapiens
ATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 284 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 285 - protein, 152 aa, Homo sapiens
AATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 286 - protein, 148 aa, Homo sapiens
ATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 287 - protein, 154 aa, Homo sapiens
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 288 - protein, 154 aa, Homo sapiens
ATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 289 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 290 - protein, 159 aa, Homo sapiens
AKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 291 - protein, 153 aa, Homo sapiens
ATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 292 - protein, 152 aa, Homo sapiens
ATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 293 - protein, 155 aa, Homo sapiens
ATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 294 - protein, 154 aa, Homo sapiens
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 295 - protein, 152 aa, Homo sapiens
ATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 296 - protein, 152 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 297 - protein, 160 aa, Homo sapiens
AKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 298 - protein, 151 aa, Homo sapiens
ATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 299 - protein, 149 aa, Homo sapiens
CAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 300 - protein, 149 aa, Homo sapiens
CAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 301 - protein, 149 aa, Homo sapiens
CVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 302 - protein, 149 aa, Homo sapiens
CVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 303 - protein, 149 aa, Homo sapiens
CVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 304 - protein, 143 aa, Homo sapiens
KKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 305 - protein, 149 aa, Homo sapiens
CVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 306 - protein, 149 aa, Homo sapiens
CTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 307 - protein, 149 aa, Homo sapiens
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 308 - protein, 149 aa, Homo sapiens
CTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 309 - protein, 127 aa, Homo sapiens
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 310 - protein, 159 aa, Homo sapiens
KGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 311 - protein, 134 aa, Homo sapiens
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 312 - protein, 160 aa, Homo sapiens
AHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 313 - protein, 156 aa, Homo sapiens
ASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 314 - protein, 117 aa, Homo sapiens
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 315 - protein, 114 aa, Homo sapiens
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 316 - protein, 170 aa, Homo sapiens
IIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 317 - protein, 172 aa, Homo sapiens
NSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 318 - protein, 171 aa, Homo sapiens
IIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 319 - protein, 171 aa, Homo sapiens
IIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 320 - protein, 146 aa, Homo sapiens
AAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 321 - protein, 151 aa, Homo sapiens
MYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 322 - protein, 146 aa, Homo sapiens
AAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 323 - protein, 149 aa, Homo sapiens
ATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 324 - protein, 151 aa, Homo sapiens
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 325 - protein, 151 aa, Homo sapiens
MYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 326 - protein, 151 aa, Homo sapiens
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 327 - protein, 150 aa, Homo sapiens
NVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 328 - protein, 152 aa, Homo sapiens
MYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 329 - protein, 162 aa, Homo sapiens
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 330 - protein, 152 aa, Homo sapiens
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 331 - protein, 152 aa, Homo sapiens
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 332 - protein, 148 aa, Homo sapiens
RRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 333 - protein, 162 aa, Homo sapiens
ASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 334 - protein, 138 aa, Homo sapiens
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 335 - protein, 138 aa, Homo sapiens
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 336 - protein, 117 aa, Homo sapiens
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 337 - protein, 161 aa, Homo sapiens
DNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 338 - protein, 161 aa, Homo sapiens
DNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 339 - protein, 161 aa, Homo sapiens
DNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 340 - protein, 161 aa, Homo sapiens
DNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 341 - protein, 161 aa, Homo sapiens
DNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 342 - protein, 150 aa, Homo sapiens
ATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 343 - protein, 151 aa, Homo sapiens
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 344 - protein, 152 aa, Homo sapiens
ATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 345 - protein, 149 aa, Homo sapiens
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 346 - protein, 145 aa, Homo sapiens
STKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 347 - protein, 123 aa, Homo sapiens
ATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 348 - protein, 170 aa, Homo sapiens
MIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 349 - protein, 144 aa, Homo sapiens
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 350 - protein, 144 aa, Homo sapiens
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 351 - protein, 144 aa, Homo sapiens
QCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 352 - protein, 146 aa, Homo sapiens
AAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 353 - protein, 100 aa, Homo sapiens
ATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 354 - protein, 577 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 355 - protein, 556 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 356 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 357 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 358 - protein, 18 aa, Homo sapiens
MHSSALLCCLVLLTGVRA

SEQ ID NO: 359 - protein, 20 aa, Homo sapiens
MYRMQLLSCIALSLALVTNS

SEQ ID NO: 360 - protein, 25 aa, Homo sapiens
MGVLLTQRTLLSLVLALLFPSMASM

SEQ ID NO: 361 - protein, 18 aa, Homo sapiens
MASLWLLSCFSLVGAAFG

SEQ ID NO: 362 - protein, 15 aa, Homo sapiens
MNLLLILTFVAAAVA

SEQ ID NO: 363 - protein, 24 aa, Homo sapiens
MALWMRLLPLLALLALWGPDPAAA

SEQ ID NO: 364 - protein, 12 aa, Homo sapiens
EPKSCDKTHTCP

SEQ ID NO: 365 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 366 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 106 aa, Homo sapiens [sequence omitted]

SEQ ID NO: 368 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 222 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 372 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 374 - protein, 12 aa, Homo sapiens
ESKYGPPCPSCP

SEQ ID NO: 375 - protein, 13 aa, Homo sapiens
AESKYGPPCPSCP

SEQ ID NO: 376 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 377 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 378 - protein, 106 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 379 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 380 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 381 - protein, 223 aa, Homo sapiens
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 382 - protein, 222 aa, Homo sapiens
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 383 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 384 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 385 - protein, 230 aa, Homo sapiens
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 386 - protein, 229 aa, Homo sapiens
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 387 - protein, 107 aa, Homo sapiens [sequence omitted]

SEQ ID NO: 388 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 389 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 390 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 391 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 393 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 395 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 396 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 397 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 398 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 399 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 400 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 401 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 402 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 403 - protein, 8 aa, synthetic construct
GGGGGGGS

SEQ ID NO: 404 - protein, 16 aa, synthetic construct
GGGGGGGSGGGGGGGS

SEQ ID NO: 405 - protein, 24 aa, synthetic construct
GGGGGGGSGGGGGGGSGGGGGGGS

SEQ ID NO: 406 - protein, 5 aa, synthetic construct
GGGGS

SEQ ID NO: 407 - protein, 10 aa, synthetic construct
GGGGSGGGGS

SEQ ID NO: 408 - protein, 15 aa, synthetic construct
GGGGSGGGGSGGGGS

SEQ ID NO: 409 - protein, 4 aa, synthetic construct
GGGS

SEQ ID NO: 410 - protein, 8 aa, synthetic construct
GGGSGGGS

SEQ ID NO: 411 - protein, 12 aa, synthetic construct
GGGSGGGSGGGS

SEQ ID NO: 412 - protein, 445 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 413 - protein, 425 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 414 - protein, 445 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 415 - protein, 425 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 416 - protein, 452 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 417 - protein, 432 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 418 - protein, 518 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRKGEELFTGVVPILVELDGDVNGHKFSVSGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKRPAANDENYAASV

SEQ ID NO: 419 - protein, 498 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRKGEELFTGVVPILVELDGDVNGHKFSVSGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKRPAANDENYAASV

SEQ ID NO: 420 - protein, 447 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 421 - protein, 464 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 422 - protein, 453 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 423 - protein, 466 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 424 - protein, 449 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 425 - protein, 454 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 426 - protein, 442 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 427 - protein, 449 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 428 - protein, 456 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 429 - protein, 453 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 430 - protein, 427 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 431 - protein, 444 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 432 - protein, 433 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 433 - protein, 446 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 434 - protein, 429 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 435 - protein, 434 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 436 - protein, 422 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 437 - protein, 429 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 438 - protein, 436 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 439 - protein, 433 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 440 - protein, 444 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 441 - protein, 444 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 442 - protein, 446 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 443 - protein, 463 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 444 - protein, 452 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 445 - protein, 465 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 446 - protein, 448 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 447 - protein, 453 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 448 - protein, 451 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 449 - protein, 441 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 450 - protein, 448 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 451 - protein, 455 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 452 - protein, 452 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 453 - protein, 424 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 454 - protein, 424 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 455 - protein, 426 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 456 - protein, 443 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 457 - protein, 432 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 458 - protein, 445 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 459 - protein, 428 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 460 - protein, 433 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 461 - protein, 431 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 462 - protein, 421 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 463 - protein, 428 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 464 - protein, 435 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 465 - protein, 432 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 466 - protein, 444 aa, synthetic construct; identical to SEQ ID NO: 440

SEQ ID NO: 467 - protein, 424 aa, synthetic construct; identical to SEQ ID NO: 453

SEQ ID NO: 468 - protein, 1527 aa, Severe acute respiratory syndrome coronavirus 2
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 469 - protein, 1508 aa, Severe acute respiratory syndrome coronavirus 2
SAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 470 - protein, 13 aa, Mus sp.
VPRDCGCKPCICT

SEQ ID NO: 471 - protein, 107 aa, Mus sp.
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTK

SEQ ID NO: 472 - protein, 107 aa, Mus sp.
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 473 - protein, 106 aa, Mus sp.
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 474 - protein, 214 aa, Mus sp.
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 475 - protein, 213 aa, Mus sp.
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 476 - protein, 227 aa, Mus sp.
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 477 - protein, 227 aa, Mus sp.; identical to SEQ ID NO: 476

SEQ ID NO: 478 - protein, 16 aa, Mus sp.
EPRGPTIKPCPPCKCP

SEQ ID NO: 479 - protein, 134 aa, Mus sp.
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVT

SEQ ID NO: 480 - protein, 83 aa, Mus sp.
LTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 481 - protein, 82 aa, Mus sp.
LTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 482 - protein, 217 aa, Mus sp.
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 483 - protein, 216 aa, Mus sp.
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 484 - protein, 233 aa, Mus sp.
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 485 - protein, 232 aa, Mus sp.
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 486 - protein, 217 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 487 - protein, 216 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 488 - protein, 233 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 489 - protein, 232 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 490 - protein, 217 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 491 - protein, 216 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 492 - protein, 233 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 493 - protein, 232 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 494 - protein, 449 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 495 - protein, 441 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 496 - protein, 449 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 497 - protein, 441 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 498 - protein, 457 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 499 - protein, 448 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 500 - protein, 429 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 501 - protein, 421 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 502 - protein, 429 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 503 - protein, 421 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 504 - protein, 437 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 505 - protein, 428 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 506 - protein, 427 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 507 - protein, 431 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 508 - protein, 423 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 509 - protein, 427 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 510 - protein, 431 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 511 - protein, 423 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 512 - protein, 421 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 513 - protein, 438 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 514 - protein, 431 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 515 - protein, 443 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 516 - protein, 428 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 517 - protein, 424 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 518 - protein, 426 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 519 - protein, 431 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 520 - protein, 422 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 521 - protein, 417 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 522 - protein, 407 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 523 - protein, 411 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 524 - protein, 403 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 525 - protein, 407 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 526 - protein, 411 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 527 - protein, 403 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 528 - protein, 401 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 529 - protein, 418 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 530 - protein, 411 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 531 - protein, 423 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 532 - protein, 408 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 533 - protein, 404 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 534 - protein, 406 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 535 - protein, 411 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 536 - protein, 402 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 537 - protein, 397 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 538 - protein, 426 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 539 - protein, 426 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 540 - protein, 420 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 541 - protein, 437 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 542 - protein, 430 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 543 - protein, 442 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 544 - protein, 427 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 545 - protein, 423 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 546 - protein, 425 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 547 - protein, 416 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 548 - protein, 406 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 549 - protein, 406 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 550 - protein, 400 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 551 - protein, 417 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 552 - protein, 410 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 553 - protein, 422 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 554 - protein, 407 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 555 - protein, 403 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 556 - protein, 405 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 557 - protein, 396 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 558 - protein, 423 aa, synthetic construct; identical to SEQ ID NO: 508

SEQ ID NO: 559 - protein, 403 aa, synthetic construct; identical to SEQ ID NO: 524

SEQ ID NO: 560 - protein, 431 aa, synthetic construct
MYRMQLLSCIALSLALVTNSEPRGPTIKPCLLCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 561 - protein, 411 aa, synthetic construct
EPRGPTIKPCLLCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 562 - protein, 431 aa, synthetic construct; identical to SEQ ID NO: 507

SEQ ID NO: 563 - protein, 411 aa, synthetic construct; identical to SEQ ID NO: 523

SEQ ID NO: 564 - protein, 253 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 565 - protein, 233 aa, synthetic construct; identical to SEQ ID NO: 488

SEQ ID NO: 566 - protein, 245 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSP

SEQ ID NO: 567 - protein, 225 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSP

SEQ ID NO: 568 - protein, 248 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 569 - protein, 228 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 570 - protein, 53 aa, Homo sapiens
MCSYYHMKKRSVSGCNITIFAVMFSHLSAGKSPCGNQANVLCISRLEFVQYQS

SEQ ID NO: 571 - protein, 47 aa, Homo sapiens
MKKRSVSGCNITIFAVMFSHLSAGKSPCGNQANVLCISRLEFVQYQS

SEQ ID NO: 572 - protein, 23 aa, Homo sapiens
KSPCGNQANVLCISRLEFVQYQS